(19) United States
(12) Patent Application Publication
(10) Pub. No.: US 2010/0256357 A1
(43) Pub. Date: Oct. 7, 2010

(54) HETEROCYCLES USEFUL AS INHIBITORS OF CARBONIC ANHYDRASE

(75) Inventors: Wesley Kwan Mung Chong, Encinitas, CA (US); Andrew M. Haidle, Cambridge, MA (US); Martha Alicia Ornelas, San Diego, CA (US); Lin Li, Shanghai (CN); Seiji Nukui, San Diego, CA (US); Eugene Yuanjin Rui, San Diego, CA (US); Min Teng, San Diego, CA (US); William Francois Vernier, Oceanside, CA (US); Joe Zhongxiang Zhou, San Diego, CA (US)

Correspondence Address: PFIZER INC, 10555 SCIENCE CENTER DRIVE, SAN DIEGO, CA, 92121, US

(73) Assignee: Agouron Pharmaceuticals, Inc.

(21) Appl. No.: 12/376,677

(22) PCT Filed: Jul. 30, 2007
(86) PCT No.: PCT/IB07/02276

§ 371 Date: Apr. 28, 2009

Related U.S. Application Data
(60) Provisional application No. 60/836,812, filed on Aug. 9, 2006.

(51) Int. Cl. C07D 413/06; C07D 249/12; C07D 413/12; C07D 401/06; C07D 403/06

(52) U.S. Cl. 540/544; 548/266.8; 544/132; 546/272.4; 544/366; 544/105

(57) ABSTRACT

Sulfonamides and pharmaceutical compositions containing the compounds useful in controlling intraocular pressure are disclosed. Methods for controlling intraocular pressure through administration of the compositions are also disclosed.

### FIELD OF INVENTION

The invention relates to heterocycles, methods for their preparation, pharmaceutical compositions containing these compounds, and methods of using these compounds and compositions for inhibiting carbonic anhydrase, and thereby lowering intraocular pressure and treating glaucoma.

### BACKGROUND OF INVENTION

Glaucoma is a disease of the eye characterized by a progressive loss of visual field due to irreversible damage to the optic nerve to the point where if untreated, may result in total blindness. This loss of visual field, in one form of primary open angle glaucoma, or POAG, is associated with a sustained increase in the intraocular pressure (IOP) of the diseased eye. Moreover, elevated intraocular pressure without visual field loss is thought to be indicative of the early stages of this form of POAG.

There are a number of therapies that target reducing the elevated IOP associated with this form of POAG. The most common are the topical administration of a beta adrenergic antagonist or a muscarinic agonist. These treatments while effective in lowering IOP can also produce significant undesirable side effects. Another treatment of POAG is the systemic administration of carbonic anhydrase inhibitors. For example, U.S. Pat. Nos. 5,679,670, 4,797,413, 4,847,289 and 4,731,368 disclose topically dosed thiophene sulfonamides which lower IOP by inhibiting carbonic anhydrase. However, these compounds may also bring about unwanted side effects, such as nausea, dyspepsia, fatigue and metabolic acidosis. The compounds of the present invention are heterocycles which inhibit carbonic anhydrase activity, and are thereby useful for lowering intraocular pressure and treating glaucoma, without producing significant systemic side effects when delivered topically to the eye.

### SUMMARY OF INVENTION

The invention relates to heterocycles, methods for their preparation, pharmaceutical compositions containing these compounds, and methods of using these compounds and compositions for inhibiting carbonic anhydrase, and thereby lowering intraocular pressure and treating glaucoma.

In one aspect, the invention relates to a compound having formula I:

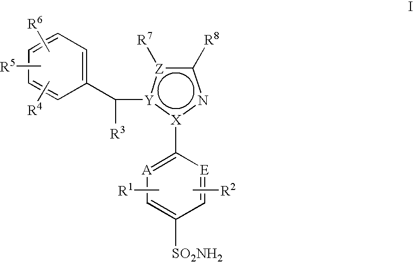

or a pharmaceutically acceptable salt or solvate thereof, wherein:

A and E are each independently C or N;

X is C; Y is N; and Z is C; or

X is N; Y is C; and Z is C or N;

R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkanyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R₆, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(O)(CH₂)ᵤ(3-10 membered heterocyclyl);

R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R⁷ and R⁸ are optionally each independently H, F, Cl, Br, I or (C₁-C₅)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), or

R⁷ and R⁸ form a fused 6-membered heteroaryl ring;

R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜO(CH₂)ᵤ(C₁-C₁₂)alkyl, (CH₂)ₜCN, (CH₂)ₜCF₃, (CF₂)ₜCF₃; (CH₂)ₜOCF₃, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹⁰ and R¹¹ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜC₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹²R¹³, (CH₂)ₜNR¹²R¹³, (CH₂)ₜ(C₈-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹² and R¹³ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₃-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₃-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NH₂, (CH₂)ₜC(═O)NH(C₁-C₁₂)alkyl, (CH₂)ₜ(═O)N(C₁-C₁₂)alkyl)₂, (CH₂)ₜNH₂, (CH₂)ₜNH(C₁-C₁₂)alkyl, (CH₂)ₜN((C₁-C₁₂)alkyl)₂, (CH₂)ₜ(C₈-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl);

R¹, R², R³, R⁴, R⁵, R⁶, R⁷, R⁸, R⁹, R¹⁰, R¹¹, R¹², R¹³, R¹⁴ and R¹⁵ groups are each optionally independently substituted with 1 to 5 R¹⁶ groups;

R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃₁ (CH₂)ₜOCF₃, SO₂(C₁-C₁₂)alkyl, (CH₂)ₜOC(═O)R⁹, (CH₂)ₜC(═O)OR⁸, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₈-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl; (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R², (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)₆(C₆-C₁₀)aryl, (CH₂)ₜ(3-10 membered heterocyclyl) and (CH₂)ₜC(═O)(CH₂)ᵤ(3-10 membered heterocyclyl);

t, u and v are each independently 0, 1, 2, 3, 4, 5 or 6; and

w is 1, 2 or 3.

In another aspect, the invention relates to the compound of formula I, wherein:

A and E are each independently C or N;

X is C; Y is N; and Z is C; or

X is N; is C; and Z is C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is optionally H or (C₁-C₃)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN; (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein:

A and E are C;

X is C; Y is N; and Z is C; or

X is N; Y is C; and Z is C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₈)alkyl, CF₃ or OCHF₂;

R⁷ is optionally H or (C₁-C₅)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CF₂)ᵤ(3-10 membered heterocyclyl); or

R⁷ and R⁸ form a fused pyridinyl ring.

In another aspect, the invention relates to the compound of formula I, wherein:

A is C; and E is N;

X is C; Y is N; and Z is C; or

X is N; Y is C; and Z is C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R¹ is optionally H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₈-C₁₀)aryl, (CH₂)₅(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein:

A and E are each independently C or N;

X is C; Y is N; and Z is C;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is optionally H or (C₁-C₃)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein:

A and E are C;

X is C; Y is N; and Z is C;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is optionally H or (C₁-C₈)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁸, (CH₂)ₜC(═O)OR⁹, (CH₂)ₜC(═O)R⁹, (CH₂)ₜOR⁹, (CH₂)SO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein:

A is C; and E is N;

X is C; Y is N; and Z is C;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is optionally H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein:

A and E are each independently C or N;

X is N; Y is C; and Z is C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is optionally H or (C₁-C₈)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜ(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein:

A and E are C;

X is N; Y is C; and Z is C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is optionally H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein:

A is C; and E is N;

X is N; Y is C; and Z is C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₈)alkyl, CF₃ or OCHF₂;

R⁷ is optionally H or (C₁-C₈)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₃-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula I, wherein the (C₆-C₁₀)aryl is a phenyl or naphthylene group, each optionally substituted with 1 to 5 R¹⁶ groups.

In another aspect, the invention relates to the compound of formula I, wherein the 3-10 membered heterocyclyl is an: oxetane, azetidine, tetrahydrofuran, pyrrolidine, 2,5-dihydro-1H-pyrrole, 1,3-dioxalane, isoxazolidine, oxazolidine, pyrazolidine, imidazolidine, pyrrolidin-2-one, tetrahydrothiophene-1,1-dioxide, pyrrolidine-2,5-dione, tetrahydro-2H-pyran, piperidine, 1,2,3,6-tetrahydropyridine, 1,4-dioxane, morpholine, piperazine, thiomorpholine, piperidin-2-one, piperidin-4-one, thiomorpholine-1,1-dioxide, 1,3-oxazinan-2-one, morpholin-3-one, piperazine-2-one, azepane, 1,4-oxazepane, 1,4-diazepane, azepan-2-one, 1,4-diazepan-5-one, quinuclidine, 2-aza-bicyclo[2.2.1]heptane, 8-aza-bicyclo[3.2.1]octane, 5-oxa-2-aza-bicyclo[2.2.1]heptane, 2-oxa-5-aza-bicyclo[2.2.1]heptan-3-one, 2-oxa-5-aza-bicyclo[2.2.2]octan-3-one, 1-methyl-5,6-pyrrolyl-7-oxa-bicyclo[2.2.1]heptane, 6-aza-bicyclo[3.2.1]octane, 3,8-diaza-bicyclo-[3:2.1]octan-2-one, 2,2-dimethyl-tetrahydro-3aH-[1,3]dioxolo[4,5-c]pyrrole, 3,3-cyclohexylpyrrolidine, 1,5-diaxo-9-azaspiro[5.5]undecane, octahydro-1H-isoindole, decahydroquinoline, decahydro-isoquinoline, octahydropyrrolo[1,2a]pyrazine, octahydro1H-pyrido[1,2a]pyrazine, octahydro-pyrrolo[3,4-c]-pyridine-3-one, decahydropyrazino[1,2-a]azepine, furan, 1H-pyrrole, isoxazole, oxazole, 1H-pyrazole, 1H-imidazole, thiazole, 1,2,4-oxadiazole, 1,3,4-oxadiazole, 4H-1,2,4-triazole, 1H-tetrazole, pyridine, pyridazine, pyrimidine, pyrazine, pyridine-2(1H)-one, 1,4,5,6-tetrahydro-cyclopenta[c]pyrazole, 6,7-dihydro-5H-pyrrolo[2,1-c][1,2,4]triazole, 2,3-dihydroimidazo-[2,1-b]-thiazole, imidazo[2,1-b][1,3,4-c]pyridine, 4,5,6,7-tetrahydro-3H-imidazo[4,5-c]pyridine, 5,6,7,8-tetrahydroimidazo[1,5-a]pyrazine, 4,5,6,7-tetrahydrothiazole[5,4-c]pyridine, 5,6,7,8-tetrahydro-[1,2,4]triazolo[4,3-a]pyrazine, quinoline, isoquinoline, 2,3-dihydrobenzofuran, 5,6,7,8-tetrahydro-quinoline, 3,4-dihydro-1H-isochromene, 1,2,3,4-tetrahydroisoquinoline, 4H-benzo[d][1,3]dioxane, 5,6,7,8-tetrahydropyrido[3,4-d]pyrimidine, benzofuran, 1H-indole, benzo[d]oxazole, 1H-benzo[d]-imidazole, H-imidazo[1,2-a]pyridine, imidazo[1,2-a]pyrimidine, 5,6,7,8-tetrahydroimidazo[1,5-a]-pyrazine-3(2H)-one, 2,3,4,5-tetrahydro-1H-benzo[d]azepine, 2,3,4,5-tetrahydrobenzo[f][1,4]ox-azepine, 5,6,7,8-tetrahydro-4H-isoxazolo[4,3-d]azepine or 6,7,8,9-tetrahydro-2H-[1,2,4]triazolo-[4,3-g][1,4]diazepin-3(5H)-one group, wherein each 3-10 membered heterocyclyl group is optionally substituted with 1 to 5 R¹⁶ groups.

In another aspect, the invention relates to the compound of formula II:

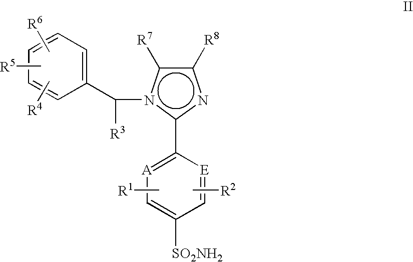

or a pharmaceutically acceptable salt or solvate thereof, wherein:

A and E are each independently C or N;

R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CF₂)ₜ(C₈-C₁₂)cycloalkynyl, (CH₂)ₜCN, (CH₂)ₜCF₃, (Ch₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl);

R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN; (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₈-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl);

R⁷ and R⁸ are optionally each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁸, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), or

R⁷ and R⁸ form a fused 6-membered heteroaryl ring;

R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(CH₂)ᵤ(C₁-C₁₂)alkyl, (CH₂)ₜCN, (CH₂)ₜCF₃, (CF₂)ₜCF₃, (CH₂)ₜOCF₃, (CH₂)ₜC(═O)NR¹⁰R¹¹, (CH₂)ₜNR¹⁰R¹¹, (CH₂)ₜ(C₆-C₁₀)aryl, (CF₂)ₜ(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹⁰ and R¹¹ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹²R¹³, (CH₂)ₜNR¹²R¹³, (CH₂)ₜ(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl);

R¹² and R¹³ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (C₃-C₁₂)ₜC₃-C₁₂)cycloalkyl(C₆C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NH₂, (CH₂)ₜC(═O)NH(C₁-C₁₂)alkyl, (CH₂)ₜC(═O)N((C₁-C₁₂)alkyl)₂, (CH₂)ₜNH₂, (CH₂)ₜNH(C₁-C₁₂)alkyl, (CH₂)ₜN((C₁-C₁₂)alkyl)₂, (CH₂)ₜ(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl);

R¹, R², R³, R⁴, R⁵, R⁶, R⁷, R⁸, R⁹, R¹⁰, R¹¹, R¹², R¹³, R¹⁴ and R¹⁵ groups are each optionally independently substituted with 1 to 5 R¹⁶ groups;

R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₅-C₁₀)aryl, (CH₂)ₜ(3-10 membered heterocyclyl) or (CH₂)ₜC(═)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(=O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₈-C₁₀)aryl, (CH₂)ₜO(CH₂)ᵤ(C₆-C₁₀)aryl, (CH₂)ₜ(3-10 membered heterocyclyl) and (CH₂)ₜC(═O)(CH₂)ᵤ(3-10 membered heterocyclyl);

t, u and v are each independently 0, 1, 2, 3, 4, 5 or 6; and

w is 1, 2 or 3.

In another aspect, the invention relates to the compound of formula II, wherein:

A and E are each independently C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)^{t}C(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula II, wherein:

A and E are C;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each Independently H, F, Cl, Br, I, (C₁-C₆)alkyl, CF₃ or OCHF₂;

R⁷ is H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)₂OR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula II, wherein:

A is C; and E is N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCH, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula II, wherein the (C₆-C₁₀)aryl is a phenyl or naphthylene group, each optionally substituted with 1 to 5 R¹⁶ groups.

In another aspect, the invention relates to the compound of formula II, wherein the 3-10 membered-heterocyclyl is an oxetane, azetidine, tetrahydrofuran, pyrrolidine, 2,5-dihydro-1H-pyrrole, 1,3-dioxalane, isoxazolidine, oxazolidine, pyrazolidine, imidazolidine, pyrrolidin-2-one, tetrahydrothiophene-1,1-dioxide, pyrrolidine-2,5-dione, tetrahydro-2H-pyran, piperidine, 1,2,3,6-tetrahydropyridine, 1,4-dioxane, morpholine, piperazine, thiomorpholine, piperidin-2-one, piperidin-4-one, thiomorpholine-1,1-dioxide, 1,3-oxazinan-2-one, morpholin-3-one, piperazine-2-one, azepane, 1,4-oxazepane, 1,4-diazepane, azepan-2-one, 1,4-diazepan-5-one, quinuclidine, 2-aza-bicyclo[2.2.1]heptane, 8-aza-bicyclo[3.2.1]octane, 5-oxa-2-aza-bicyclo[2.2.1]heptane, 2-oxa-5-aza-bicyclo[2.2.1]heptan-3-one, 2-oxa-5-aza-bicyclo[2.2.2]octan-3-one, 1-methyl-5,6-pyrrolyl-7-oxa-bicyclo[2.2.1]heptane, 6-aza-bicyclo[3.2.1]octane, 3,8-diaza-bicyclo-[3.2.1]octan-2-one, 2,2-dimethyl-tetrahydro-3aH-[1,3]dioxolo[4,5-c]pyrrole, 3,3-cyclohexylpyrrolidine, 1,5-diaxo-9-azaspiro[5.5]undecane, octahydro-1H-isoindole, decahydroquinoline, decahydro-isoquinoline, octahydropyrrolo[1,2a]pyrazine, octahydro1H-pyrido[1,2a]pyrazine, octahydro-pyrrolo[3,4-c]-pyridine-3-one, decahydropyrazino[1,2-a]azepine, furan, 1H-pyrrole, isoxazole, oxazole, 1H-pyrazole, 1H-imidazole, thiazole, 1,2,4-oxadiazole, 1,3,4-oxadiazole, 4H-1,2,4-triazole, 1H-tetrazole, pyridine, pyridazine, pyrimidine, pyrazine, pyridine-2(1H)-one, 1,4,5,6-tetrahydro-cyclopenta[c]pyrazole, 6,7-dihydro-5H-pyrrolo[2,1-c][1,2,4]triazole, 2,3-dihydroimidazo-[2,1-b]-thiazole, imidazo[2,1-b][1,3,4-c]pyridine, 4,5,6,7-tetrahydro-3H-imidazo[4,5-c]pyridine, 5,6,7,8-tetrahydroimidazo[1,5-a]pyrazine, 4,5,6,7-tetrahydrothiazole[5,4-c]pyridine, 5,6,7,8-tetrahydro-[1,2,4]triazolo[4,3-a]pyrazine, quinoline, isoquinoline, 2,3-dihydrobenzofuran, 5,6,7,8-tetrahydro-quinoline, 3,4-dihydro-1H-isochromene, 1,2,3,4-tetrahydroisoquinoline, 4H-benzo[d][1,3]dioxane, 5,6,7,8-tetrahydropyrido[3,4-d]pyrimidine, benzofuran, 1H-indole, benzo[d]oxazole, 1H-benzo[d]-imidazole, H-imidazo[1,2-a]pyridine, imidazo[1,2-a]pyrimidine, 5,6,7,8-tetrahydroimidazo[1,5-a]-pyrazine-3(2H)-one, 2,3,4,5-tetrahydro-1H-benzo[d]azepine, 2,3,4,5-tetrahydrobenzo[f][1,4]ox-azepine, 5,6,7,8-tetrahydro-4H-isoxazolo[4,3-d]azepine or 6,7,8,9-tetrahydro-2H-[1,2,4]triazolo-[4,3-g][1,4]diazepin-3(5H)-one group, wherein each 3-10 membered heterocyclyl group is optionally substituted with 1 to 5 R¹⁶ groups.

In another aspect, the invention relates to the compound of formula III:

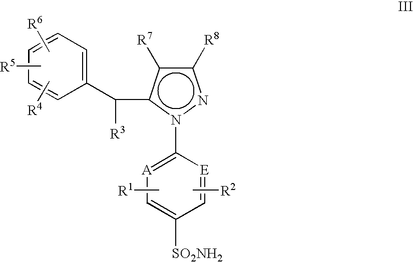

or a pharmaceutically acceptable salt or solvate thereof, wherein:

A and E are each independently C or N;

R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁸, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃; (CH₂)ₜO(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl);

R⁷ and R⁸ are optionally each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), or

R⁷ and R⁸ form a fused 6-membered heteroaryl ring;

R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜO(CH₂)ᵤ(C₁-C₁₂)alkyl, (CH₂)ₜCN, (CH₂)ₜCF₃, (CF₂)ₜCF₃, (CH₂)ₜOCF₃, (CH₂)ₜC(═O)NR¹⁰R¹¹, (CH₂)ₜNR¹⁰R¹¹, (CH₂)ₜ(C₅-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹⁰ and R¹¹ are each independently H, (C₂-C₁₂)alkenyl (C₂-C₁₂)alkynyl, (CH₂)ₜ(C₃-C₁₂)cycloalkyl, (CH₂)ₜ(C₃-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹²R¹³, (CH₂)ₜNR¹²R¹³, (CH₂)^{t}(C₂)ₜ(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl);

R¹² and R¹³ are each independently H, (C₁-C₁₂)alkyl, (C₂-C₁₂)alkenyl, (c₂-C₁₂)alkynyl, (CH₂)ₜ(C₃-C₁₂)cycloalkyl, (CH₂)ₜ(C₃-C₁₂)cycloalkenyl, (CH₂)ₜ(C₆-C₁₂)cycloalkynyl, (CH₂)ₜ(C₃-C₁₂)cycloalkyl(C₈-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₆-C₁₀)aryl, (CH₂)ₜO(CH₂)ᵤ(C₆₈-C₁₀)aryl, (CH₂)ₜ(3-10 membered heterocyclyl) or (CH₂)ₜC(═O)(CH₂)ᵤ(3-10 membered heterocyclyl);

R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NH₂, (CH₂)ₜC(═O)NH(C₁-C₁₂)alkyl, (CH₂)ₜC(═O)N((C₁-C₁₂)alkyl)₂, (CH₂)ₜNH₂, (CH₂)ₜNH(C₁-C₁₂)alkyl, (CH₂)ₜN((C₁-C₁₂)alkyl)₂, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹, R², R³, R⁴, R⁵, R⁶, R⁷, R⁸, R⁹, R¹⁰, R¹¹, R¹², R¹³, R¹⁴ and R¹⁵ groups are each optionally independently substituted with 1 to 5 R¹⁶ groups;

R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₃-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)CF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₈-C₁₀)aryl, (CH₂)ₜO(CH₂)ᵤ(C₆-C₁₀)aryl, (CH₂)ₜ(3-10 membered heterocyclyl) and (CH₂)ₜC(═O)(CH₂)ᵤ(3-10 membered heterocyclyl);

t, u and v are each independently 0, 1, 2, 3, 4, 5 or 5; and

w is 1, 2or 3,

In another aspect, the invention relates to the compound of formula III, wherein:

A and E are each independently C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₈)alkyl, CF₃ or OCHF₂;

R⁷ is H or (C₁-C₃)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula III, wherein:

A and E are C;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)CF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)₂SO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the invention relates to the compound of formula III, wherein:

A is C; and E is N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂;

R⁷ is H or (C₁-C₆)alkyl; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or

R⁷ and R⁸ form a fused pyridinyl ring.

In another aspect, the Invention relates to the compound of formula III, wherein the (C₆-C₁₀)aryl is a phenyl or naphthylene group, each optionally substituted with 1 to 5 R¹⁰ groups.

In another aspect, the invention relates to the compound of formula III, wherein the 3-10 membered heterocyclyl is an: oxetane, azetidine, tetrahydrofuran, pyrrolidine, 2,5-dihydro-1H-pyrrole, 1,3-dioxalane, isoxazolidine, oxazolidine, pyrazolidine, imidazolidine, pyrrolidin-2-one, tetrahydrothiophene-1,1-dioxide, pyrrolidine-2,5-dione, tetrahydro-2H-pyran, piperidine, 1,2,3,5-tetrahydropyridine, 1,4-dioxane, morpholine, piperazine, thiomorpholine, piperidin-2-one, piperidin-4-one, thiomorpholine-1,1-dioxide, 1,3-oxazinan-2-one, morpholin-3-one, piperazine-2-one, azepane, 1,4-oxazepane, 1,4-diazepane, azepan-2-one, 1,4-diazepan-5-one, quinuclidine, 2-aza-bicyclo[2.2.1]heptane, 8-aza-bicyclo[3.2.1]octane, 5-oxa-2-aza-bicyclo[2.2.1]heptane, 2-oxa-5-aza-bicyclo[2.2.1]heptan-3-one, 2-oxa-5-aza-bicyclo[2.2.2]octan-3-one, 1-methyl-5,6-pyrrolyl-7-oxa-bicyclo[2.2.1]heptane, 6-aza-bicyclo[3.2.1]octane, 3,8-diaza-bicyclo-[3.2.1]octan-2-one; 2,2-dimethyl-tetrahydro-3aH-[1,3]dioxolo[4,5-c]pyrrole, 3,3-cyclohexylpyrrolidine, 1,5-diaxo-9-azaspiro[5.5]undecane, octahydro-1H-isoindole, decahydroquinoline, decahydro-isoquinoline, octahydropyrrolo[1,2a]pyrazine, octahydro-1H-pyrido[1,2a]pyrazine, octahydro-pyrrolo[3,4-c]-pyridine-3-one, decahydropyrazino[1,2-a]azepine, furan, 1H-pyrrole, isoxazole, oxazole, 1H-pyrazole, 1H-imidazole, thiazole, 1,2,4-oxadiazole, 1,3,4-oxadiazole, 4H-1,2,4-triazole, 1H-tetrazole, pyridine, pyridazine, pyrimidine, pyrazine, pyridine-2(1H)-one, 1,4,5,6-tetrahydro-cyclopenta[c]pyrazole, 6,7-dihydro-5H-pyrrolo[2,1-c][1,2,4]triazole, 2,3-dihydroimidazo-[2,1-b]-thiazole, imidazo[2,1-b][1,3,4-c]pyridine, 4,5,6,7-tetrahydro-3H-imidazo[4,5-c]pyridine, 5,6,7,8-tetrahydroimidazo[1,5-a]pyrazine, 4,5,6,7-tetrahydrothiazole[5,4-c]pyridine, 5,6,7,8-tetrahydro-[1,2,4]triazolo[4,3-a]pyrazine, quinoline, isoquinoline, 2,3-dihydrobenzofuran, 5,6,7,8-tetrahydro-quinoline, 3,4-dihydro-1H-isochromene, 1,2,3,4-tetrahydroisoquinoline, 4H-benzo[d][1,3]dioxane, 5,6,7,8-tetrahydropyrido[3,4-d]pyrimidine, benzofuran, 1H-indole, benzo[d]oxazole, 1H-benzo[d]-imidazole, H-imidazo[1,2-a]pyridine, imidazo[1,2-a]pyrimidine, 5,6,7,8-tetrahydroimidazo[1,5-a]-pyrazine-3(2H)-one, 2,3,4,5-tetrahydro-1H-benzo[d]azepine, 2,3,4,5-tetrahydrobenzo[f][1,4]ox-azepine, 5,6,7,8-tetrahydro-4H-isoxazolo[4,3-d]azepine or 6,7,8,9-tetrahydro-2H-[1,2,4]triazolo-[4,3-g][1,4]diazepin-3(5H)-one group, wherein each 3-10 membered heterocyclyl group is optionally substituted with 1 to 5 R¹⁶ groups.

In another aspect, the invention relates to the compound of formula IV:

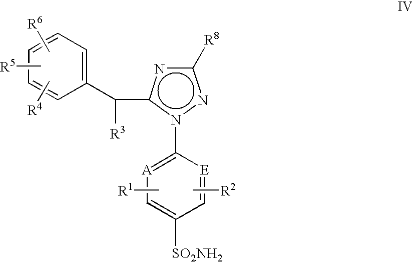

or a pharmaceutically acceptable salt or solvate thereof, wherein:

A and E are each independently C or N;

R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-CH₂)cycloalkynyl, (CH₂)ₜCN, (CH₂)ₜCF₃, (CF₂)ₜCF₃, (CH₂N)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁶, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(C₂)ᵤ(3-10 membered heterocyclyl);

R⁸ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)₆NR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₆-C₁₂)cycloalkynyl, (CH₂)ₜO(CH₂)ᵤ(C₁-C₁₂)alkyl, (CH₂)ₜCN, (CH₂)ₜCF₃, (CF₂)ₜCF₃, (CH₂)ₜOCF₃, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹⁰ and R¹¹ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹²R¹³, (CH₂)ₜNR¹²R¹³, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹² and R¹³ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₃-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₅-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₆-C₁₂)cycloalkynyl, (CH₂)ₜ(C₃-C₁₂)cycloalkyl(C₈-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (OH₂)₂SO₂R⁸, (CH₂)ₜC(═O)NH₂, (CH₂)ₜC(═O)NH(C₁-C₁₂)alkyl, (CH₂)ₜC(═O)N((C₁-C₁₂)alkyl)₂, (CH₂)ₜNH₂, (CH₂)ₜNH(C₁-C₁₂)alkyl, (CH₂)N((C₁-C₁₂)alkyl)₂, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);

R¹, R², R³, R⁴, R⁵, R⁶, R⁷, R⁸, R⁹, R¹⁰, R¹¹, R¹², R¹³, R¹⁴ and R¹⁵ groups are each optionally independently substituted with 1 to 5 R¹⁶ group;

R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₆-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)NR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) and (CH₂)ₜC(═O)(CH₂)ᵤ(3-10 membered heterocyclyl);

t, u and v are each independently 0, 1, 2, 3, 4, 5 or 6; and

w is 1, 2 or 3.

In another aspect, the Invention relates to the compound of formula IV, wherein:

A and E are each independently C or N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁸, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl).

In another aspect, the invention relates to the compound of formula IV, wherein:

A and E are C;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₈-C₁₀)aryl, (CH₂)ₜ(3-10 membered heterocyclyl) or (CH₂)ₜC(═O)(CH₂)ᵤ(3-10 membered heterocyclyl).

In another aspect, the invention relates to the compound of formula IV, wherein:

A is C; and E is N;

R¹ and R² are each independently H, F, Cl, Br, I or (C₁-C₆)alkyl;

R³ is H, F, Cl, Br, I or (C₁-C₆)alkyl;

R⁴, R⁵ and R⁶ are each independently H, F, Cl, Br, I, (C₁-C₆)alkyl, CF₃ or OCHF₂; and

R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl; (CH₂CN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜc(═O)NR¹⁰R¹¹, (CH₂)ₜNR¹⁰R¹¹, (CH₂)_{w}(C₆-C₁₀)aryl, (CH₂)ₜO(C₂)ᵤ(C₆-C₁₀)aryl, (CH₂)ₜ(3-10 membered heterocyclyl) or (CH₂)ₜC(═O)(CH₂)ᵤ(3-10 membered heterocyclyl).

In another aspect, the invention relates to the compound of formula IV, wherein the (C₆-C₁₀)aryl is a phenyl or naphthylene group, each optionally substituted with 1 to 5 R¹⁶ groups.

In another aspect, the invention relates to the compound of formula IV, wherein the 3-10 membered heterocyclyl is an: oxetane, azetidine, tetrahydrofuran, pyrrolidine, 2,5-dihydro-1H-pyrrole, 1,3-dioxalane, isoxazolidine, oxazolidine, pyrazolidine, imidazolidine, pyrrolidin-2-one, tetrahydrothiophene-1,1-dioxide, pyrrolidine-2,5-dione, tetrahydro-2H-pyran, piperidine, 1,2,3,6-tetrahydropyridine, 1,4-dioxane, morpholine, piperazine, thiomorpholine, piperidin-2-one, piperidin-4-one, thiomorpholine-1,1-dioxide, 1,3-oxazinan-2-one, morpholin-3-one, piperazine-2-one, azepane, 1,4-oxazepane, 1,4-diazepane, azepan-2-one, 1,4-diazepan-5-one, quinuclidine, 2-aza-bicyclo[2.2.1]heptane, 8-aza-bicyclo[3.2.1]octane, 5-oxa-2-aza-bicyclo[2.2.1]heptane, 2-oxa-5-aza-bicyclo[2.2.1]heptan-3-one, 2-oxa-5-aza-bicyclo[2.2.2]octan-3-one, 1-methyl-5,6-pyrrolyl-7-oxa-bicyclo[2.2.1]heptane, 6-aza-bicyclo[3.2.1]octane, 3,8-diaza-bicyclo-[3.2.1]octan-2-one, 2,2-dimethyl-tetrahydro-3aH-[1,3]dioxolo[4,5-c]pyrrole, 3,3-cyclohexylpyrrolidine, 1,5-diaxo-9-azaspiro[5.5]undecane, octahydro-1H-isoindole, decahydroquinoline, decahydro-isoquinoline, octahydropyrrolo[1,2a]pyrazine, octahydro-1H-pyrido[1,2a]pyrazine, octahydro-pyrrolo[3,4-c]-pyridine-3-one, decahydropyrazino[1,2-a]azepine, furan, 1H-pyrrole, isoxazole, oxazole, 1H-pyrazole, 1H-imidazole, thiazole, 1,2,4-oxadiazole, 1,3,4-oxadiazole, 4H-1,2,4-triazole, 1H-tetrazole, pyridine, pyridazine, pyrimidine, pyrazine, pyridine-2(1H)-one, 1,4,5,6-tetrahydro-cyclopenta[c]pyrazole, 6,7-dihydro-5H-pyrrolo[2,1-c][1,2,4]triazole, 2,3-dihydroimidazo-[2,1-b]-thiazole, imidazo[2,1-b][1,3,4-c]pyridine, 4,5,6,7-tetrahydro-3H-imidazo[4,5-c]pyridine, 5,6,7,8-tetrahydroimidazo[1,5-a]pyrazine, 4,5,6,7-tetrahydrothiazole[5,4-c]pyridine, 5,6,7,8-tetrahydro-[1,2,4]triazolo[4,3-a]pyrazine, quinoline, isoquinoline, 2,3-dihydrobenzofuran, 5,6,7,8-tetrahydro-quinoline, 3,4-dihydro-1H-isochromene, 1,2,3,4-tetrahydroisoquinoline, 4H-benzo[d][1,3]dioxane, 5,6,7,8-tetrahydropyrido[3,4-d]pyrimidine, benzofuran, 1H-indole, benzo[d]oxazole, 1H-benzo[d]-imidazole, H-imidazo[1,2-a]pyridine, imidazo[1,2-a]pyrimidine, 5,6,7,8-tetrahydroimidazo[1,5-a]-pyrazine-3(2H)-one, 2,3,4,5-tetrahydro-1H-benzo[d]azepine, 2,3,4,5-tetrahydrobenzo[f][1,4]ox-azepine, 5,6,7,8-tetrahydro-4H-isoxazolo[4,3-d]azepine or 6,7,8,9-tetrahydro-2H-[1,2,4]triazolo-[4,3-g][1,4]diazepin-3(5H)-one group, wherein each 3-10 membered heterocyclyl group is optionally substituted with 1 to 5 R¹⁶ groups.

In another aspect, the invention relates to the compound of formula I, having formula:

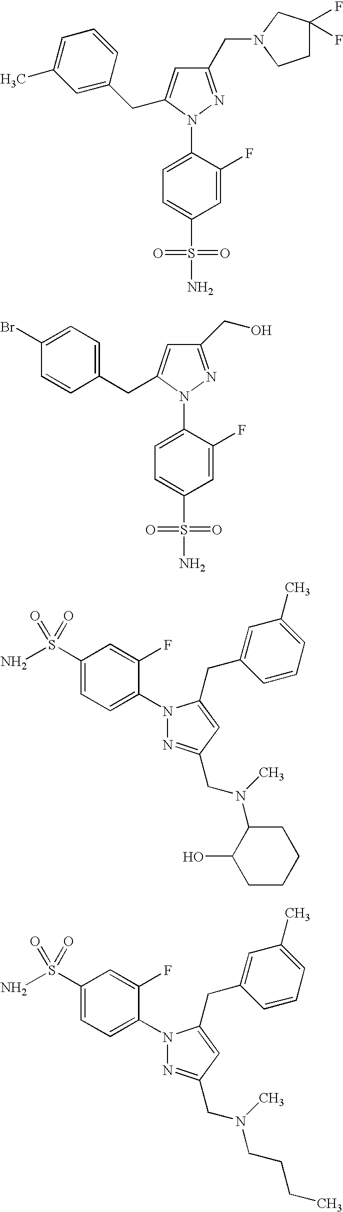

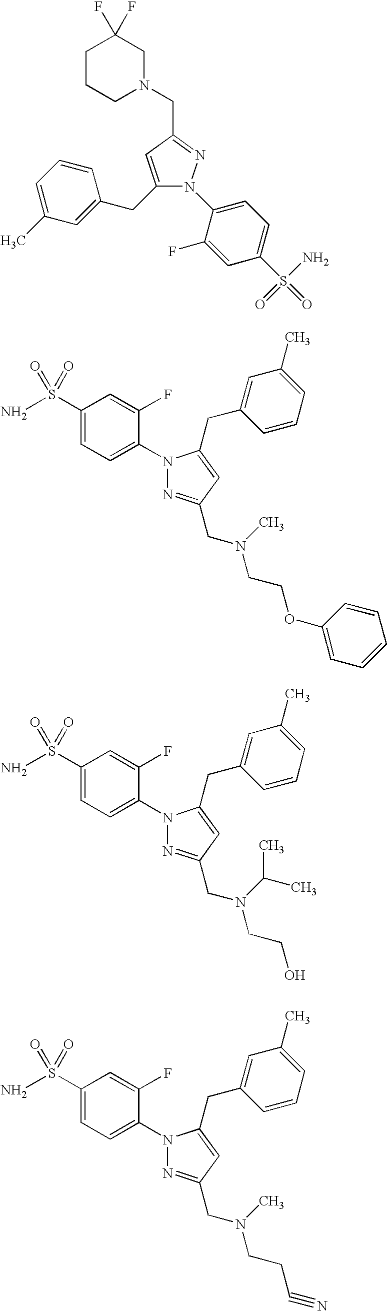

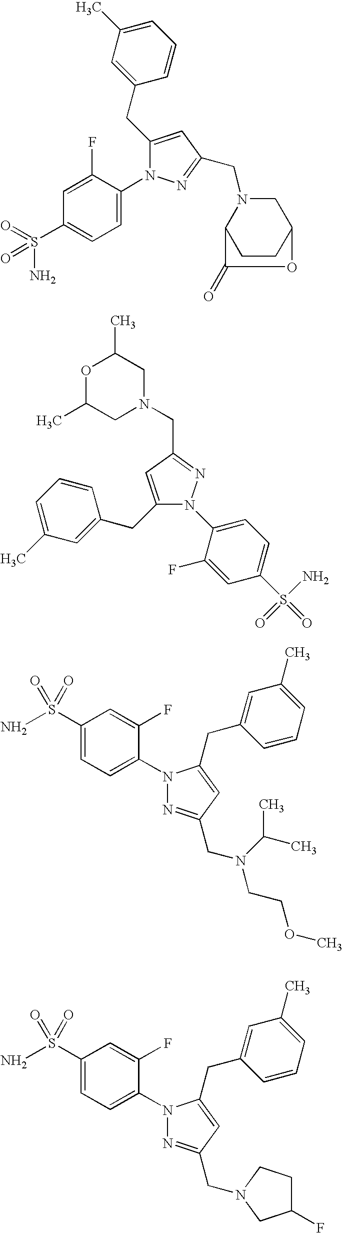

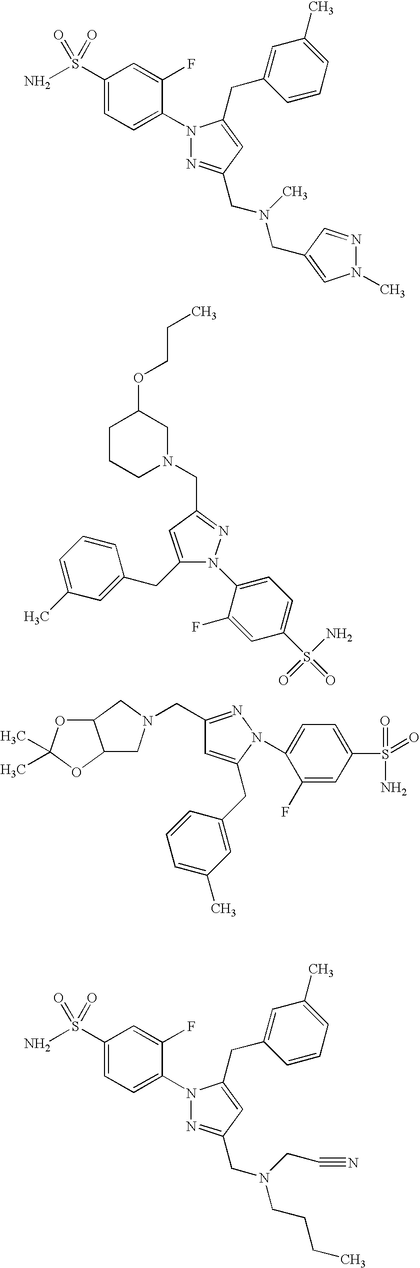

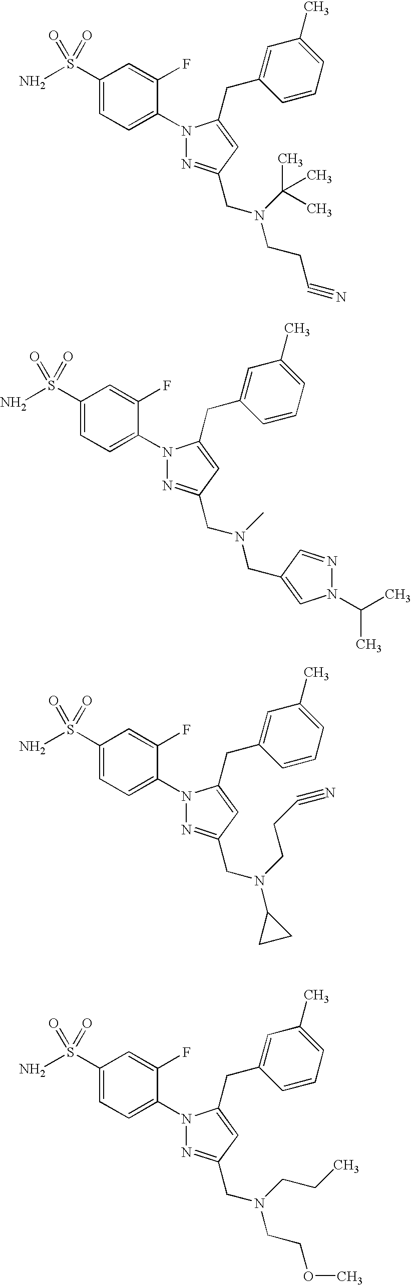

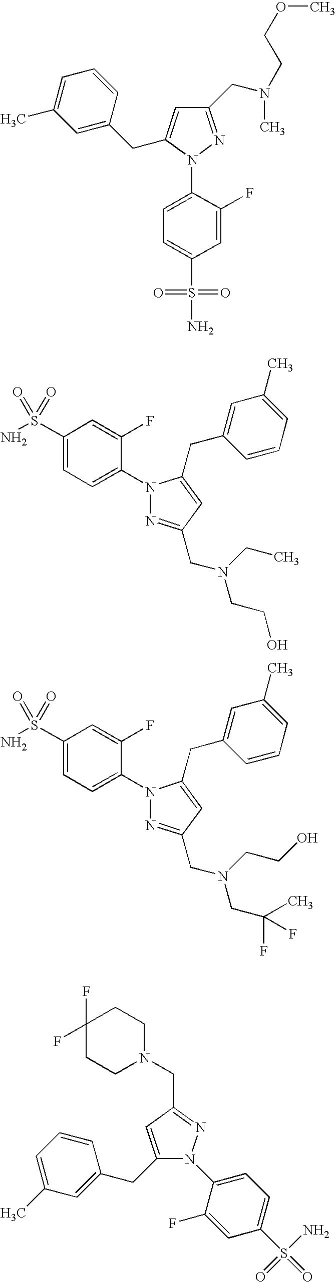

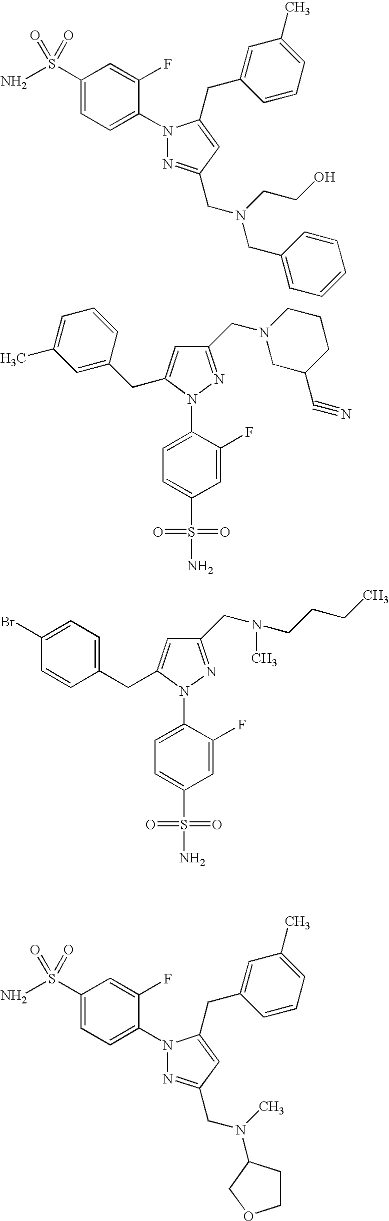

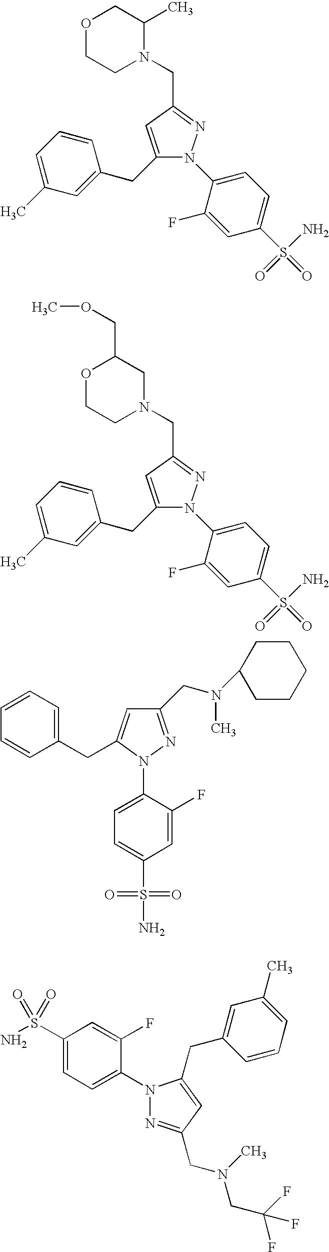

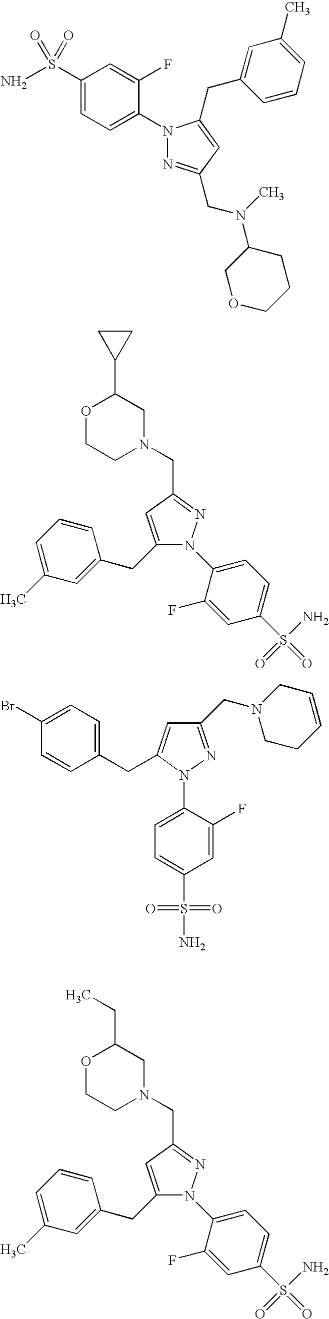

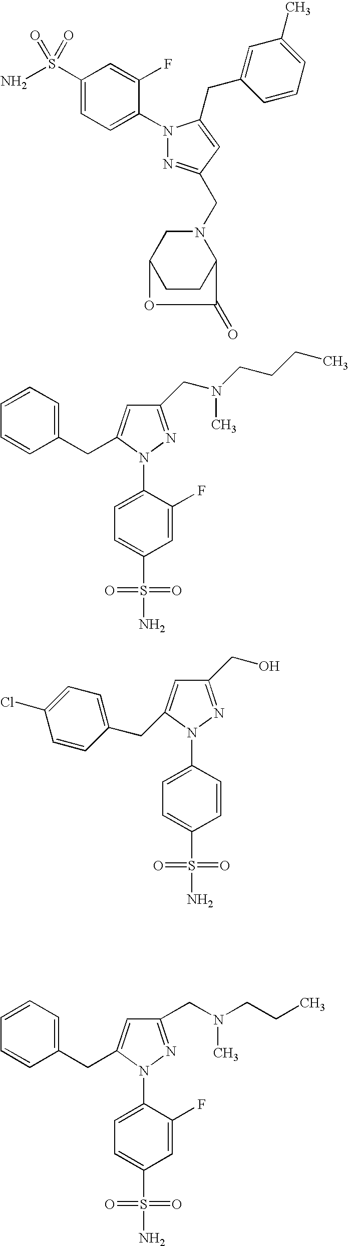

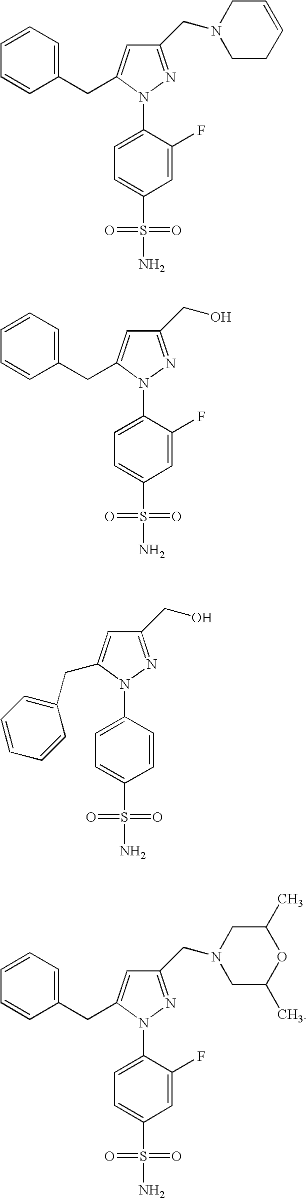

In another aspect, the invention relates to the compound of formula I, having formula:

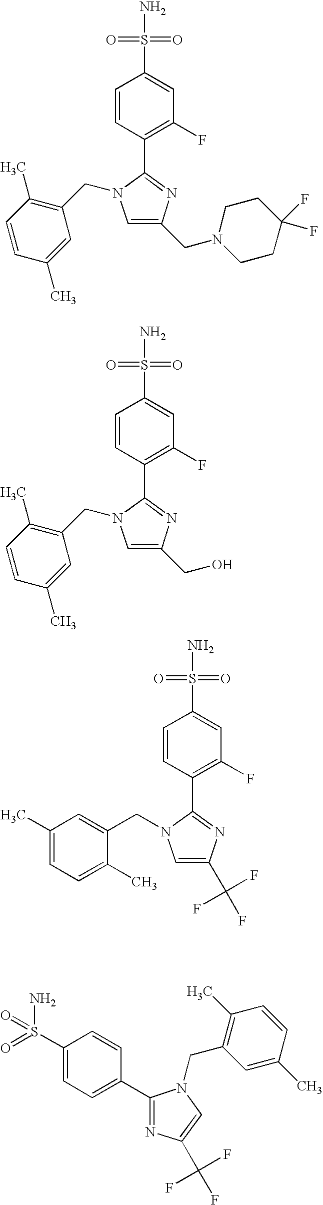

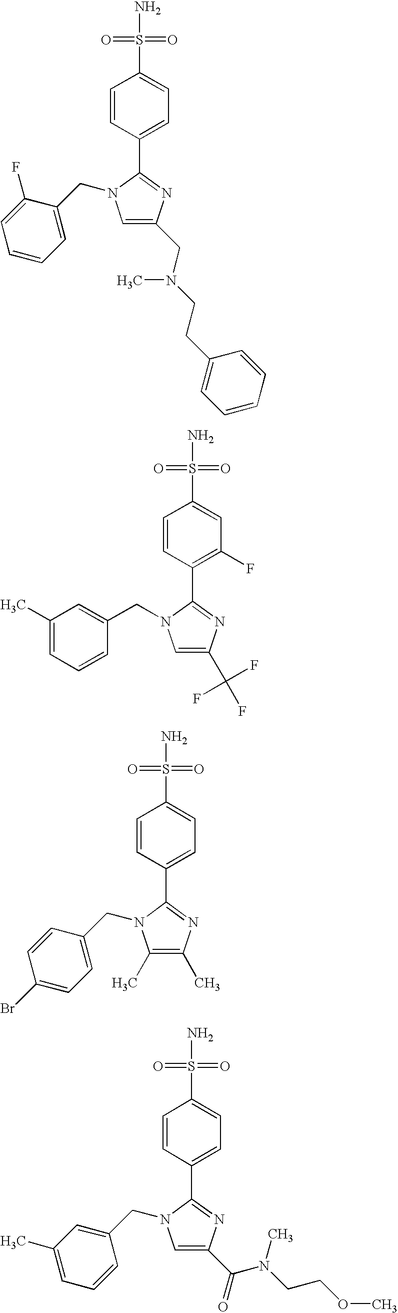

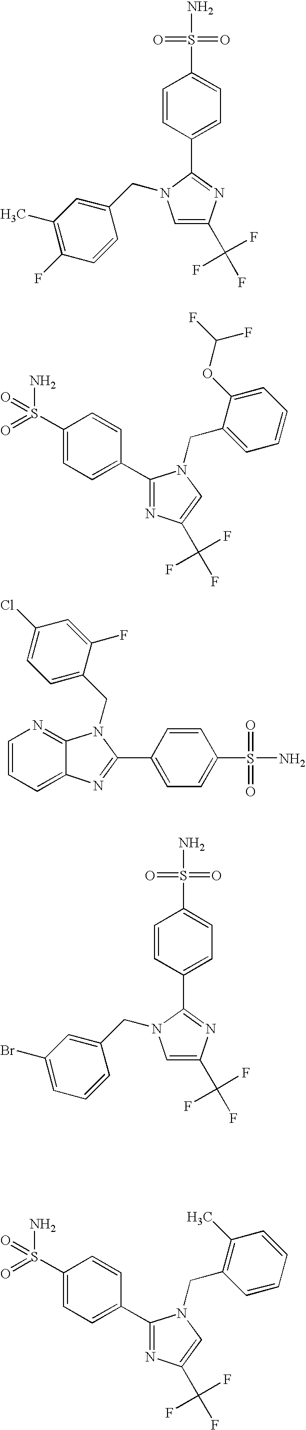

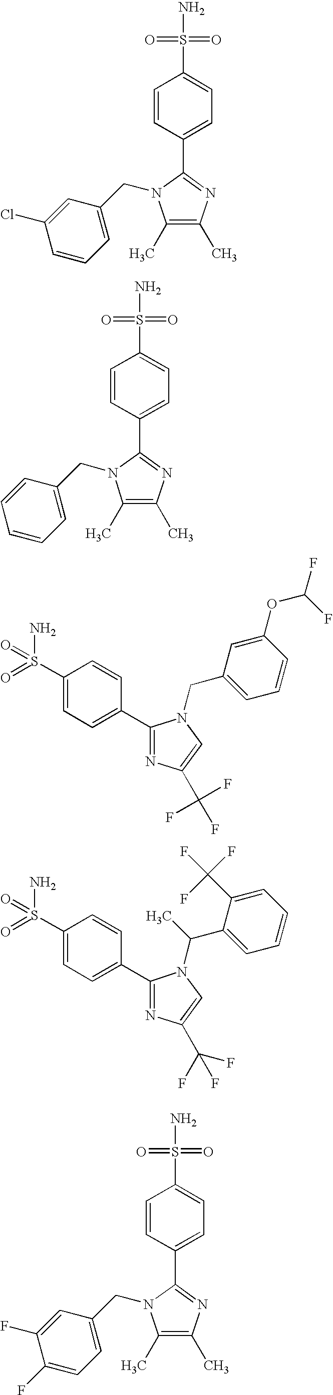

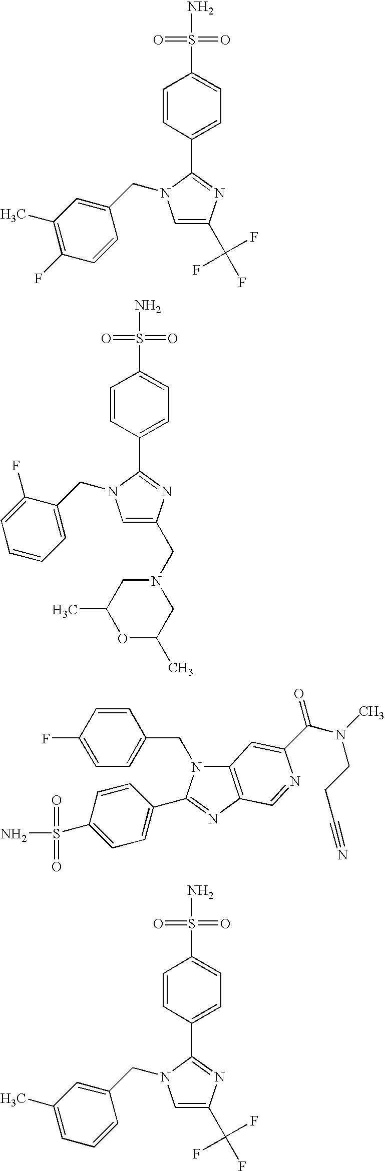

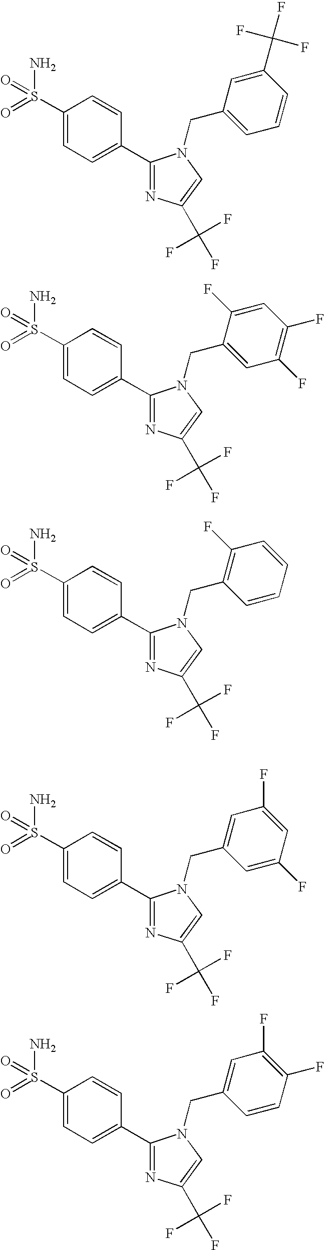

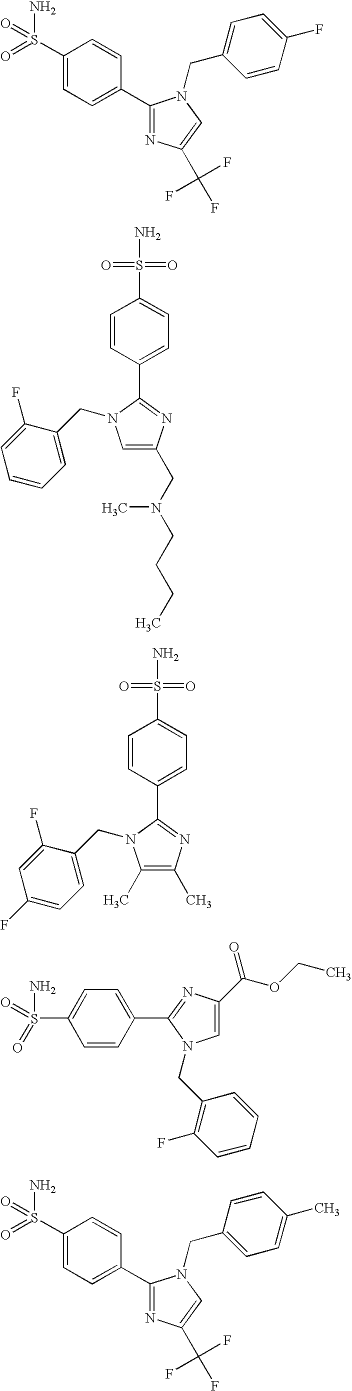

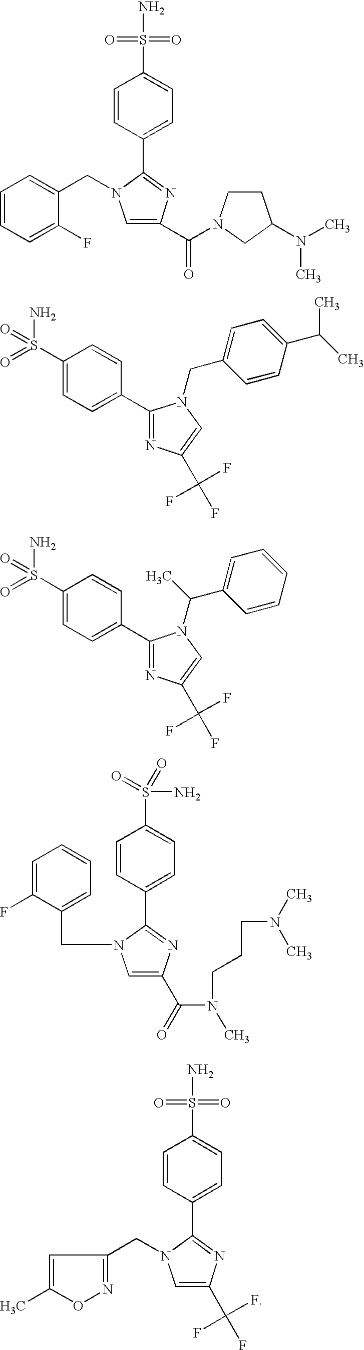

In another aspect, the invention relates to the compound of formula I, having formula:

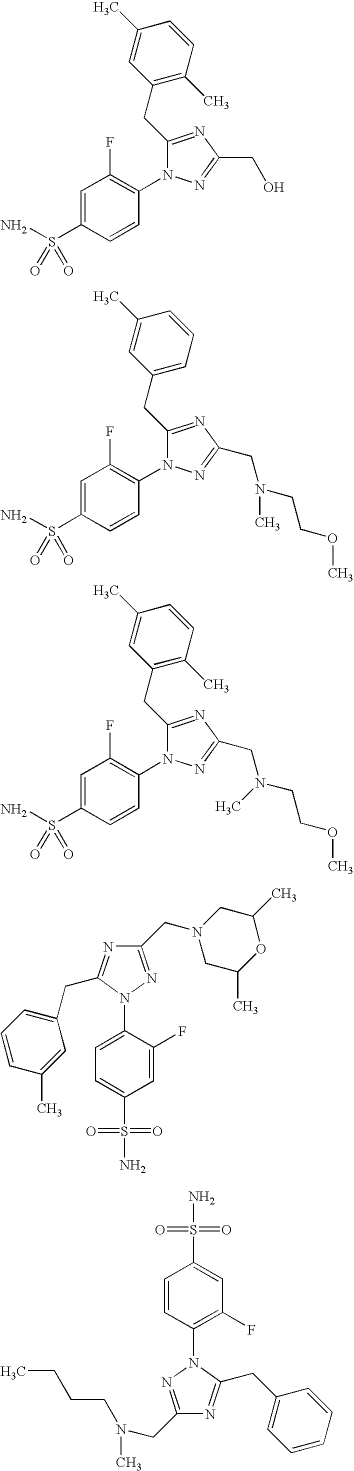

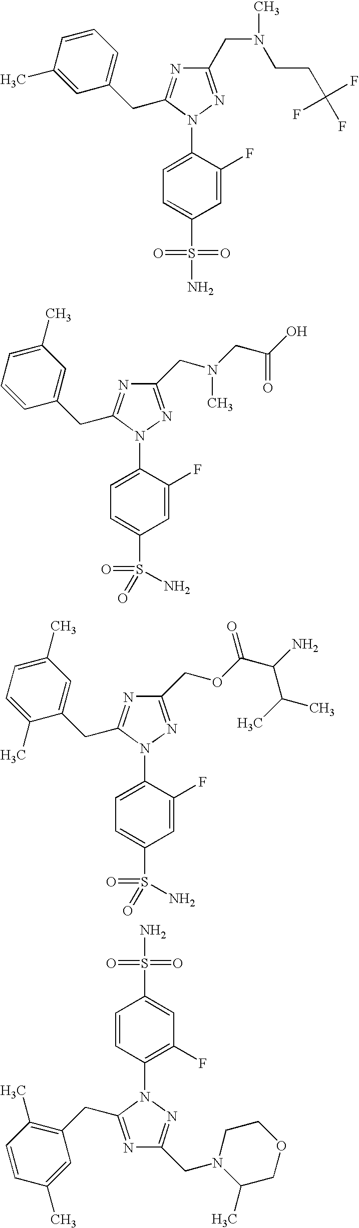

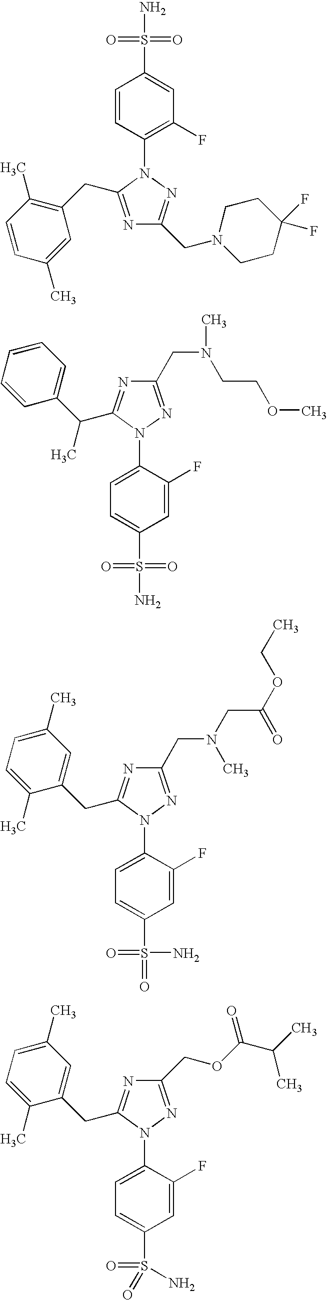

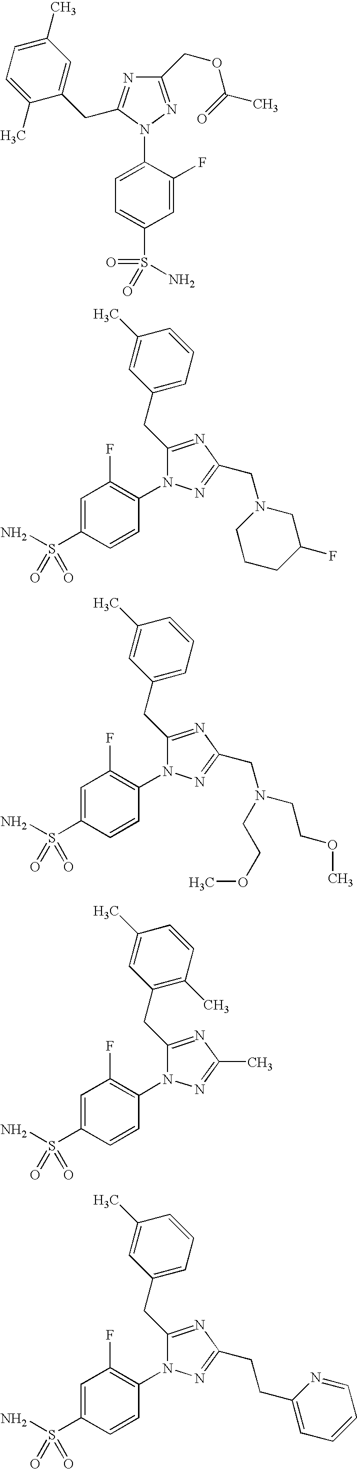

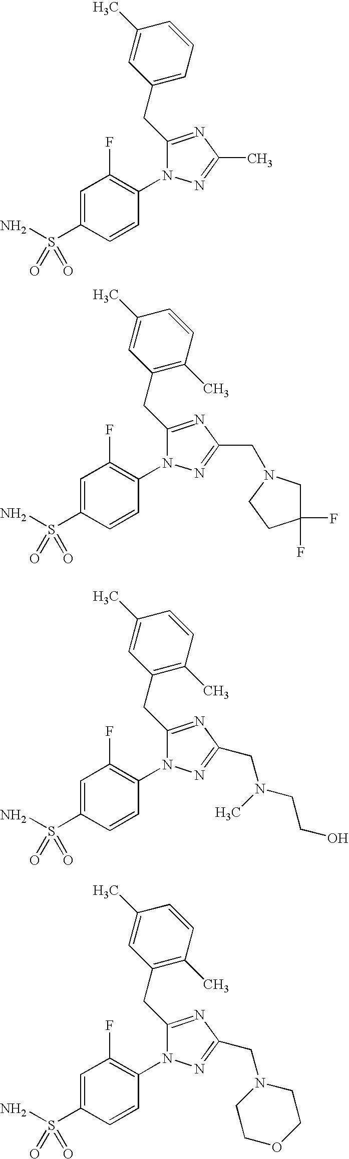

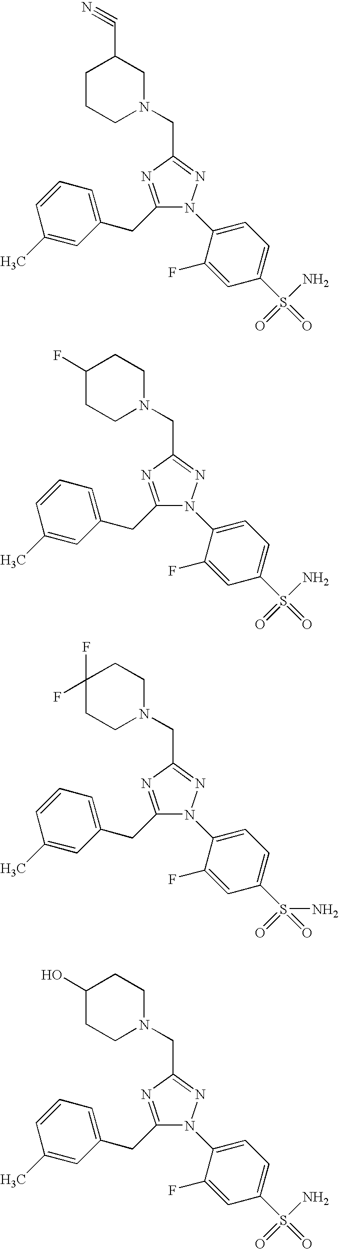

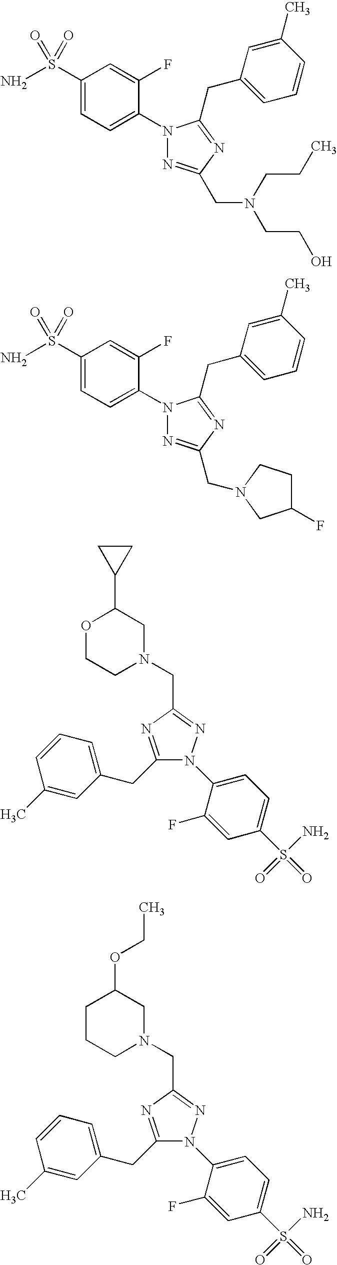

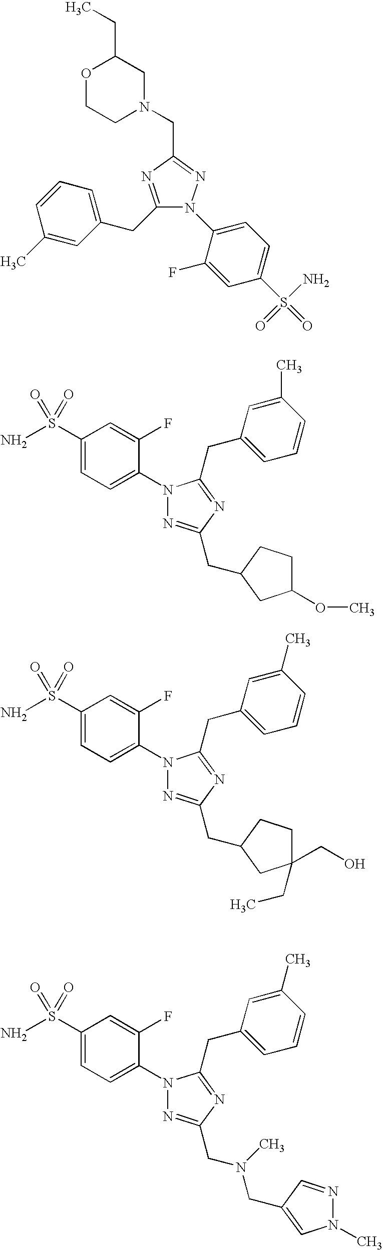

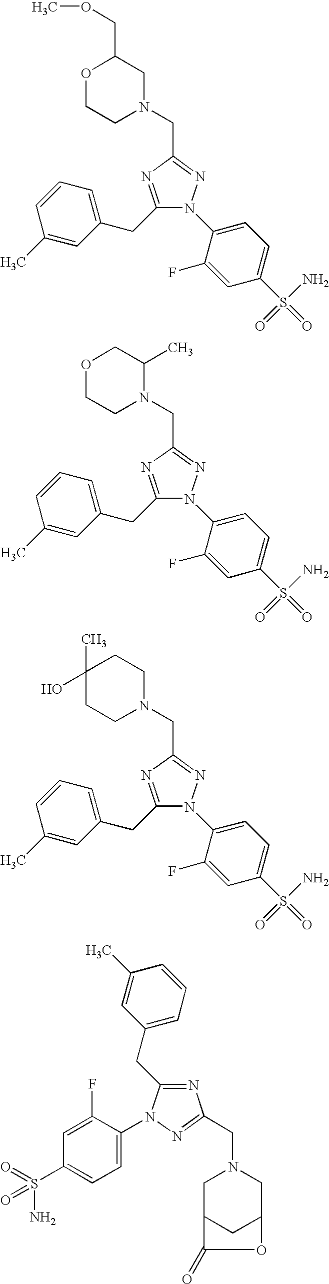

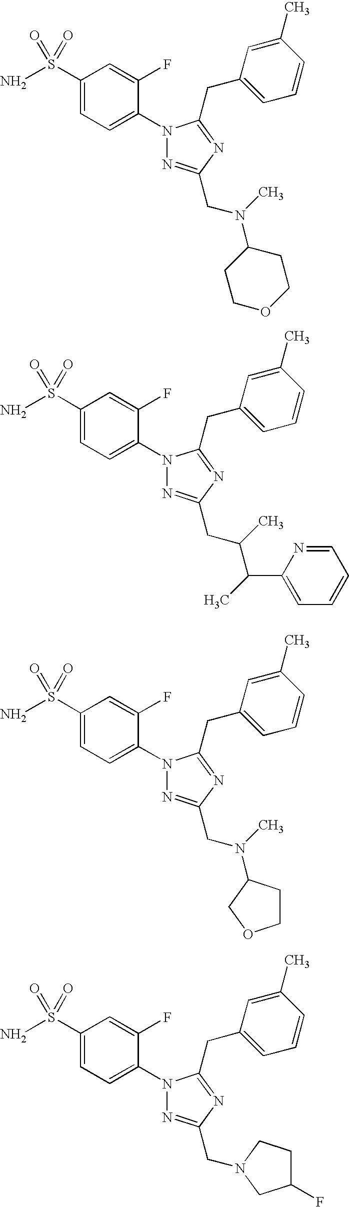

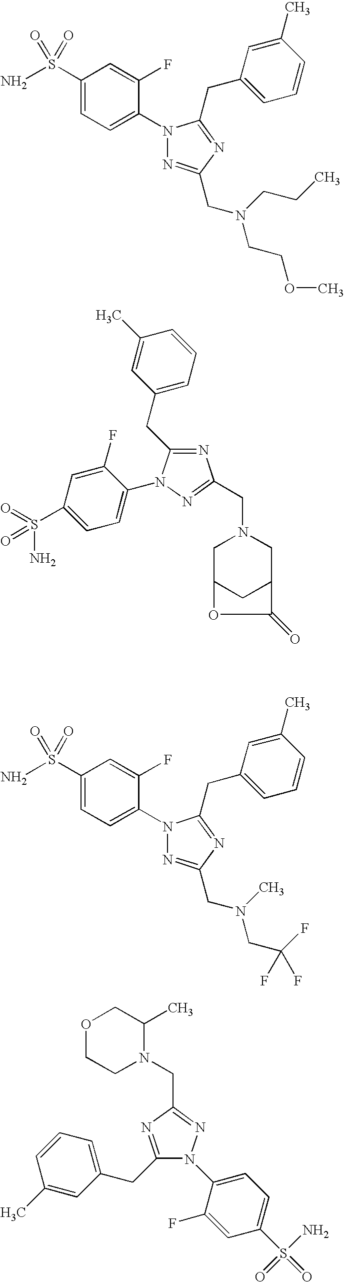

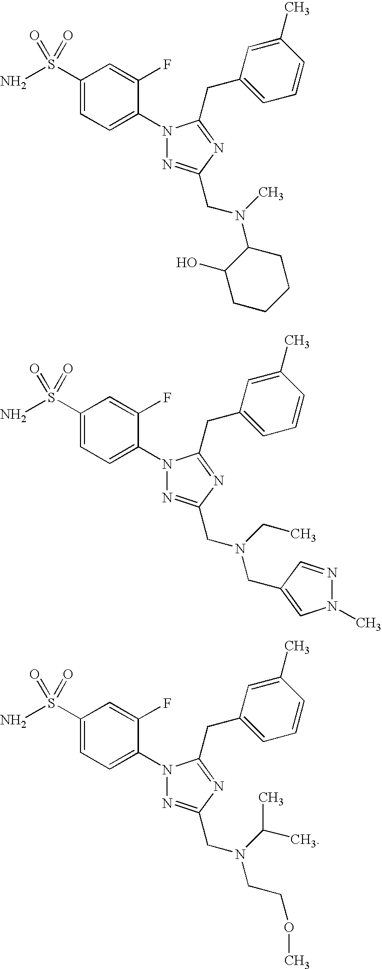

In another aspect, the invention relates to the compound of formula I, having formula XIII:

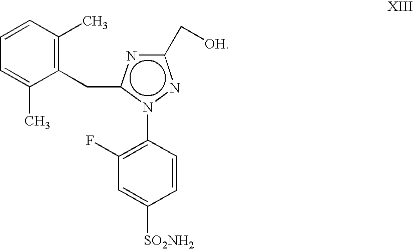

In another aspect, the invention relates to a process for preparing a compound of formula IV comprising the steps of:

i) reacting a compound of formula (V) with a compound of formula (VI) and with a compound of formula (VII) in the presence of base to provide a compound of formula (VII), wherein R¹⁷ is (C₁-C₁₂)alkyl, (CH₂)ₜ(C₆-C₁₀)aryl or (CH₂)ₜ(3-10 membered heterocyclyl); and

ii) transforming the compound of formula (VII) to the compound of formula (VI).

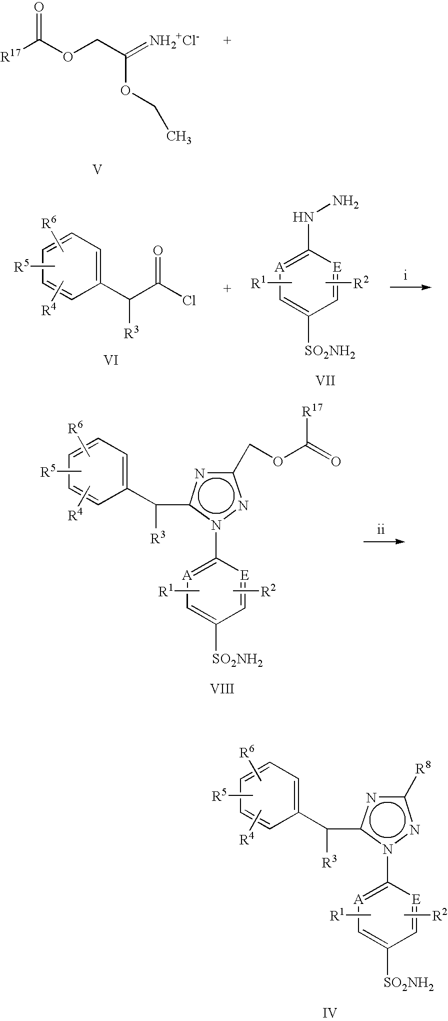

In another aspect, the invention relates to a process for preparing a compound of formula IV, wherein the first step i) the base is triethylamine; and in the second step ii) the transforming is a hydrolysis of the ester to the corresponding alcohol.

In another aspect, the invention relates to a process for preparing a compound of formula IV, wherein the compound of formula V has formula IX; the compound of formula VI has formula X; the compound of formula VII has formula XI; the compound of formula VIII has formula XII; and the compound of formula IV has formula XIII.

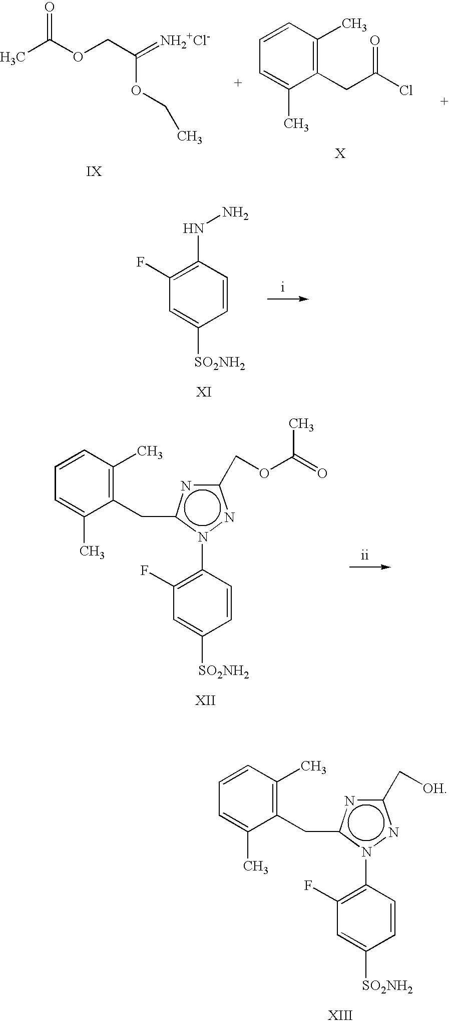

In another aspect, the invention relates to the compound of formula XIII:

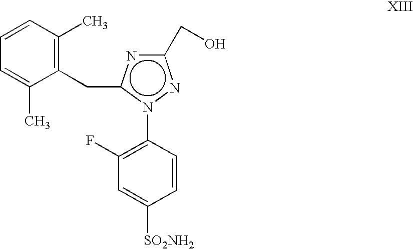

prepared by the process of:

i) reacting a compound of formula (V) with a compound of formula (VI) and with a compound of formula (VII) in the presence of base to provide a compound of formula (VII), wherein R¹⁷ is (C₁-C₁₂)alkyl, (CH₂)ₜ(C₆-C₁₀)aryl or (CH₂)ₜ(3-10 membered heterocyclyl); and

ii) transforming the compound of formula (VII) to the compound of formula (VI).

In another aspect, the invention relates to the compound of formula I, II, III or IV for use as a medicament.

In another aspect, the invention relates to the compound of formula I, II, III or IV for the preparation of a medicament for treating glaucoma and ocular hypertension.

In another aspect, the invention relates to a pharmaceutical composition comprising a pharmaceutically acceptable carrier and a pharmaceutically effective amount of a compound of formula I, II, III or IV.

In another aspect, the invention relates to a pharmaceutical composition comprising a pharmaceutically acceptable carrier and a pharmaceutically effective amount of a compound of formula I, II, III or IV, in a suitable form for topical administration.

In another aspect, the invention relates to a pharmaceutical composition comprising a pharmaceutically acceptable carrier and a pharmaceutically effective amount of a compound of formula I, II, III or IV, for the treatment of glaucoma and ocular hypertension.

In another aspect, the invention relates to a pharmaceutical composition comprising a pharmaceutically acceptable carrier and a pharmaceutically effective amount of a compound of formula I, II, III or IV, wherein the compound is administered as a solution, suspension or emulsion in an ophthalmically acceptable vehicle.

In another aspect, the invention relates to a method for treating glaucoma or ocular hypertension, wherein the method comprises contacting an effective intraocular pressure reducing amount of a pharmaceutical composition comprising a pharmaceutically acceptable carrier and a pharmaceutically effective amount of a compound of formula I, II, III or IV, with the eye in order to reduce eye pressure and to maintain the pressure on a reduced level.

In another aspect, the invention relates to the compound of formula XIII for use as a medicament.

In another aspect, the invention relates to the compound of formula XIII for the preparation of a medicament for treating glaucoma and ocular hypertension.

In another aspect, the invention relates to a pharmaceutical composition comprising a pharmaceutically acceptable carrier and a pharmaceutically effective amount of the compound of formula XIII.

In another aspect, the invention relates to a pharmaceutical composition comprising a pharmaceutically acceptable carrier and a pharmaceutically effective amount of the compound of formula XIII, for topical administration.

In another aspect, the invention relates to a pharmaceutical composition comprising a pharmaceutically acceptable carrier and a pharmaceutically effective amount of the compound of formula XIII, for the treatment of glaucoma and ocular hypertension.

In another aspect, the invention relates to a pharmaceutical composition comprising a pharmaceutically acceptable carrier and a pharmaceutically effective amount of the compound of formula XIII, wherein the compound is administered as a solution, suspension or emulsion in an ophthalmically acceptable vehicle.

In another aspect, the invention relates to a method for treating glaucoma or ocular hypertension, wherein the method comprises contacting an effective intraocular pressure reducing amount of a pharmaceutical composition comprising a pharmaceutically acceptable carrier and a pharmaceutically effective amount of the compound of formula XIII, with the eye in order to reduce eye pressure and to maintain the pressure on a reduced level.

As used herein, the terms “comprising” and “including” are used in their open, non-limiting sense.

As used herein, the term “substituted,” means that the specified group or moiety bears one or more substituents. The term “unsubstituted,” means that the specified group bears no substituents.

As used herein, the term “optionally substituted” means that the specified group is unsubstituted or is substituted by one or more substituents.

As used herein, the terms “treat,” “treating” or “treatment” includes preventative (e.g., prophylactic) and palliative treatment.

As used herein, the term “pharmaceutically acceptable” means the carrier, diluent, excipients and/or salt must be compatible with the other ingredients of the formulation and not deleterious to the recipient thereof.

As used herein, the term “alkyl” means a straight or branched chain saturated hydrocarbon. Exemplary alkyl groups include but are not limited to methyl, ethyl, propyl, isopropyl, n-butyl, sec-butyl, tert-butyl, pentyl, isopentyl, neopentyl, tert-pentyl, 1-methylbutyl, 2-methylbutyl, 3-methylbutyl, hexyl, isohexyl, heptyl, octyl and the like.

As used herein, the term “alkenyl' means a straight or branched chain hydrocarbon having at least one double bond, i.e., a C═C. Exemplary alkenyl groups include but are not limited to vinyl, propenyl, butenyl, pentenyl, hexenyl, heptenyl, octenyl and the like.

As used herein, the term “alkynyl” means a straight or branched chain hydrocarbon having at least one triple bond, i.e., a C≡C. Exemplary alkynyl groups include but are not limited to acetylenyl, propargyl, butynyl, pentynyl, hexenyl, heptynyl, octynyl and the like.

As used herein, the term “cycloalkyl” means a cyclic saturated hydrocarbon. Exemplary cycloalkyl groups include but are not limited to cyclopropyl, cyclobutyl, cyclopentyl, cyclohexyl, cycloheptyl, cyclooctyl and the like.

As used herein, the term “cycloalkenyl” means a cyclic hydrocarbon having at least one double bond, i.e., a C═C. Exemplary cycloalkenyl groups include but are not limited to cyclopropenyl, cyclobutenyl, cyclopentenyl, cyclohexenyl, cycloheptenyl, cyclooctenyl and the like.

As used herein, the term “cycloalkynyl” means a cyclic hydrocarbon having at least one triple bond, i.e., a C≡C. Exemplary cycloalkynyl groups include but are not limited to cyclohexynyl, cycloheptynyl, cyclooctynyl and the like.

As used herein, the term “alkoxy” means a straight or branched chain saturated alkyl group bonded through oxygen. Exemplary alkoxy groups include but are not limited to methoxy, ethoxy, propoxy, isopropoxy, butoxy, isobutoxy, tert-butoxy, pentoxy, isopentoxy, neopentoxy, tert-pentoxy, hexoxy, isohexoxy, heptoxy, octoxy and the like.

As used herein, the term “alkylene” means a straight chain or branched chain saturated hydrocarbon wherein a hydrogen atom is removed from each of the terminal carbons. Exemplary alkylene groups include but are not limited to methylene, ethylene, propylene, butylene, pentylene, hexylene, heptylene and the like.

As used herein, the term “cycloalkylaryl” and “(CH₂)ₜ(C₃-C₁₂)cycloalkyl(C₆-C₁₀)aryl” includes linear and/or fused ring systems such as 2,3-didydro-1H-indene, 2-methyl-2,3-didydro-1H-indene, 1,2,3,4-tetrahydronaphthalene, 2-methyl-1,2,3,4-tetrahydronaphthalene, 1-cyclopentylbenzene, 1-(2-methylcyclopentyl)benzene, 1-(3-methylcyclopentyl)benzene, 1-cyclohexylbenzene, 1-(2-methylcyclohexyl)benzene, 1-(3-methylcyclohexyl)benzene, 1-(4-methylcyclohexyl)benzene, and the like.

As used herein, the term “halo” or “halogen” means fluoro, chloro, bromo or iodo.

As used herein, the term “aryl” means an organic radical derived from an aromatic hydrocarbon by removal of hydrogen. Exemplary aryl groups include but are not limited to phenyl, biphenyl, naphthyl, and the like.

As used herein, the terms “heterocyclic” and “heterocyclyl” means an aromatic or non-aromatic cyclic group containing one to four heteroatoms each independently selected from O, S and N, wherein each group has from 3 to 10 atoms in its ring system. Non-aromatic heterocyclic groups include groups having only 3 atoms in their ring system, whereas aromatic heterocyclic groups have at least 5 atoms in their ring system. Heterocyclic groups include fused ring systems such as benzo-fused rings and the like. An exemplary 3 membered heterocyclic group is aziridine; 4 membered heterocyclic group is azetidinyl (derived from azetidine); 5 membered heterocyclic group is thiazolyl; 7 membered ring heterocyclic group is azepinyl; and a 10 membered heterocyclic group is quinolinyl.

Examples of non-aromatic heterocyclic groups include but are not limited to pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl.

Examples of aromatic heterocyclic (heteroaryl) groups include but are not limited to pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

The foregoing groups may be C-attached or N-attached where such is possible. For Instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). Heterocyclic groups may be optionally, substituted on any ring carbon, sulfur or nitrogen atom(s) by one to two oxygens (oxo), per ring. An example of a heterocyclic group wherein 2 ring carbon atoms are substituted with oxo moieties is 1,1-dioxo-thiomorpholinyl.

Exemplary five to six membered heterocyclic aromatic rings having one or two heteroatoms selected independently from oxygen, nitrogen and sulfur include but are not limited to isothiazolyl, pyridinyl, pyridiazinyl, pyrimidinyl, pyrazinyl and the like.

Exemplary partially saturated, fully saturated or fully unsaturated five to eight membered heterocyclic rings having one to four heteroatoms selected independently from oxygen, sulfur and nitrogen include but are not limited to 3H-1,2-oxathiolyl, 1,2,3-oxadizaolyl, 1,2,4-oxadiazolyl, 1,2,5-oxadiazolyl and the like. Further exemplary five membered rings are furyl, thienyl, 2H-pyrrolyl, 3H-pyrroyl, pyrrolyl, 2-pyrrolinyl, 3-pyrrolinyl, pyrrolidinyl, 1,3-dioxolanyl, oxazolyl, thiazolyl, thiazolyl, imidazolyl, 2H-imidazolyl, 2-imidazolinyl, imidazolidinyl, pyrazolyl, 2-pyrazolinyl, pyrazolinyl, isoxazolyl, isothiazolyl, 1,2-dithlolyl, 1,3-dithiolyl, 3H-1,2-oxathiolyl, 1,2,3-oxadizaolyl, 1,2,4-oxadiazolyl, 1,2,5-oxadiazolyl, 1,3,4-oxadiazolyl, 1,2,3-triazolyl, 1,2,4-trizaolyl, 1,3,4-thiadiazolyl, 1,2,3,4-oxatriazolyl, 1,2,3,5-oxatrizaolyl, 3H-1,2,3-dioxazolyl, 1,2,4-dioxazolyl, 1,3,2-dioxazolyl, 5H-1,2,5-oxathiazolyl and 1,3-oxathiolyl. Further exemplary six member rings are 2H-pyranyl, 4H-pyranyl, pyridinyl, piperidinyl, 1,2-dioxinyl, 1,3-dioxinyl, 1,4-dioxanyl, morpholinyl, 1,4-dithianyl, thiomorpholinyl, pyridazinyl, pyrimidinyl, pyrazinyl, piperazinyl, 1,3,5-triazinyl, 1,2,4-triazinyl, 1,2,3-trizainyl, 1,3,5-trithianyl, 4H-1,2-oxazinyl, 2H-1,3-oxazinyl, 6H-1,3-oxazinyl, 6H-1,2-oxazinyl, 1,4-oxazinyl, 2H-1,2-oxazinyl, 4H-1,4-oxazinyl, 1,2,5-oxathiazinyl, 1,4-oxazinyl, o-isoxazinyl, p-isoxazinyl, 1,2,5-oxathiazinyl, 1,2,6-oxathiazinyl, 1,4,2-oxadiazinyl and 1,3,5,2-oxadiazinyl. Further exemplary seven membered rings are azepinyl, oxepinyl, thiepinyl and 1,2,4-diazepinyl. Further exemplary eight membered rings are cyclooctyl, cyclooctenyl and cyclooctadienyl.

Exemplary bicyclic rings are composed of two fused partially saturated, fully saturated or fully unsaturated five or six membered rings, taken independently, optionally having one to four heteroatoms selected independently from nitrogen, sulfur and oxygen are indolizinyl, indolyl, isoindolyl, 3H-indolyl, 1H-isoindolyl, indolinyl, cyclopenta(b)pyridinyl, pyrano(3,4-b)pyrrolyl, benzofuryl, isobenzofuryl, benzo(b)thienyl, benzo(c)thienyl, 1H-indazolyl, indoxazinyl; benzoxazolyl, anthranilyl, benzimidazolyl, benzthiazolyl, purinyl, 4Hquinolizinyl, quinolinyl, isoquinolinyl, cinnolinyl, phthalazinyl, quinazolinyl, quinoxalinyl, 1,8-naphthyridinyl, pteridinyl, indenyl, isoindenyl, naphthyl, tetralinyl, decalinyl, 2H-1-benzopyranyl, pyrido(3,4-b)-pyridinyl, pyrido(3,2-b)pyridinyl, pyrido(4,3-b)-pyridinyl, 2H-1,3-benzoxazinyl, 2H-1,4-benzoxazinyl, 1H-2,3-benzoxazinyl, 4H-3,1-benzoxazinyl, 2H-1,2-benzoxazinyl and 4H-1,4-benzoxazinyl.

Exemplary 3-10 membered heterocyclyl groups include but are not limited to oxetane, azetidine, tetrahydrofuran, pyrrolidine, 2,5-dihydro-1H-pyrrole, 1,3-dioxalane, isoxazolidine, oxazolidine, pyrazolidine, imidazolidine; pyrrolidin-2-one; tetrahydrothiophene-1,1-dioxide, pyrrolidine-2,5-dione, tetrahydro-2H-pyran; 1,2,3,6-tetrahydropyridine, 1,4-dioxane, morpholine, piperazine, thiomorpholine, piperidin-2-one, piperidin-4-one, thiomorpholine-1,1-dioxide, 1,3-oxazinan-2-one, morpholin-3-one, piperazine-2-one, ozepane, 1,4-oxazepane, 1,4-diazepane, azepan-2-one, 1,4-diazepan-5-one, quinuclidine, 2-aza-bicyclo[2.2.1]heptane, 8-aza-bicyclo[3.2.1]octane, 5-oxa-2-aza-bicyclo[2.2.1]heptane, 2-oxa-5-aza-bicyclo[2.2.1]heptan-3-one, 2-oxa-5-aza-bicyclo[2.2.2]octan-3-one, 1-methyl-5,6-pyrrolyl-7-oxa-bicyclo[2.2.1]heptane, 6-aza-bicyclo[3.2.1]octane, 3,8-diaza-bicyclo[3.2.1]octan-2-one, 2,2-dimethyl-tetrahydro-3aH-[1,3]dioxolo[4,5-c]pyrrole, 3,3-cyclohexylpyrrolidine, 1,5-diaxo-9-azaspiro[5.5]undecane, octahydro-1H-isoindole, decahydroquinoline, decahydroisoquinoline, octahydropyrrolo[1,2a]pyrazine, octahydro′1H-pyrido[1,2a]pyrazine, octahydropyrrolo[3,4-c]pyridine-3-one, decahydropyrazino[1,2-a]azepine, furan, 1H-pyrrole, isoxazole, oxazole, 1H-pyrazole, 1H-imidazole, thiole, 1,2,4-oxadiazole, 1,3,4-oxadiazole, 4H-1,2,4-triazole, 1H-tetrazole, pyridine, pyridazine, pyrimidine, pyrazine, pyridine-2(1H)-one, 1,4,5,6-tetrahydrocyclopenta[c]pyrazole, 6,7-dihydro-5H-pyrrolo[2,1-c][1,2,4]triazole, 2,3-dihydroimidazo[2,1-b]thiazole, imidazo[2,1-b][1,3,4-c]pyridine, 4,5,6,7-tetrahydro-3H-imidazo[4,5-c]pyridine, 5,6,7,8-tetrahydroimidazo[1,5-a]pyrazine, 4,5,6,7-tetrahydrothiazole[5,4-c]pyridine, 5,6,7,8-tetrahydro-[1,2,4]triazolo[4,3-a]pyrazine, quinoline, isoquinoline, 2,3-dihydrobenzofuran, 5,6,7,8-tetrahydroquinoline, 3,4-dihydro-1H-isochromene, 1,2,3,4-tetrahydroisoquinoline, 4H-benzo[d][1,3]dioxane, 5,6,7,8-tetrahydropyrido[3,4-d]pyrimidine, benzofuran, 1H-indole, benzo[d]oxazole, 1H-benzo[d]imidazole, H-imidazo[1,2-a]pyridine, imidazo[1,2-a]pyrimidine, 5,6,7,8-tetrahydroimidazo[1,5-a]pyrazine-3(2H)-one, 2,3,4,5-tetrahydro-1H-benzo[d]azepine, 2,3,4,5-tetrahydrobenzo[f][1,4]oxazepine, 5,6,7,8-tetrahydro-4H-isoxazolo[4,3-d]azepine and 6,7,8,9-tetrahydro-2H-[1,2,4]triazolo[4,3-g][1,4]diazepin-3(5H)-one, and are shown below:

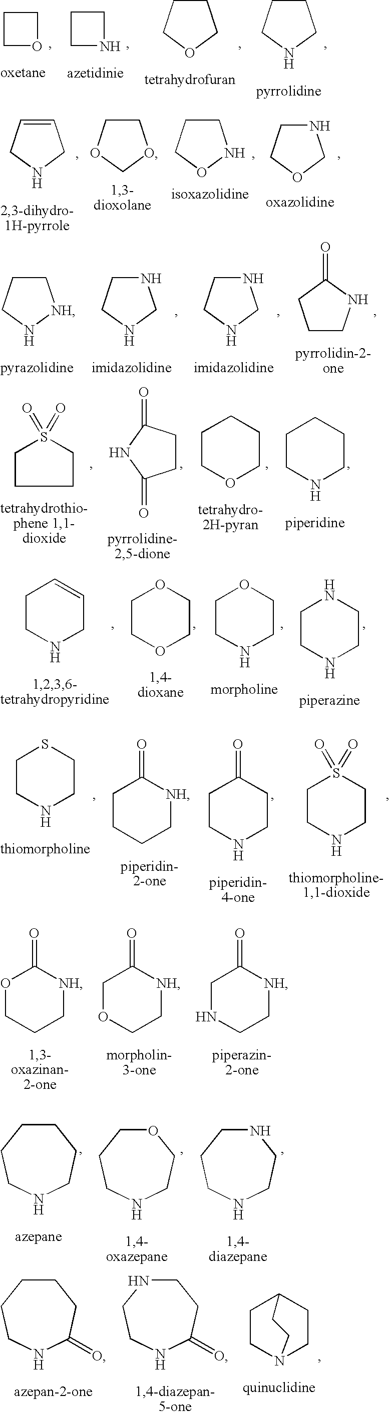

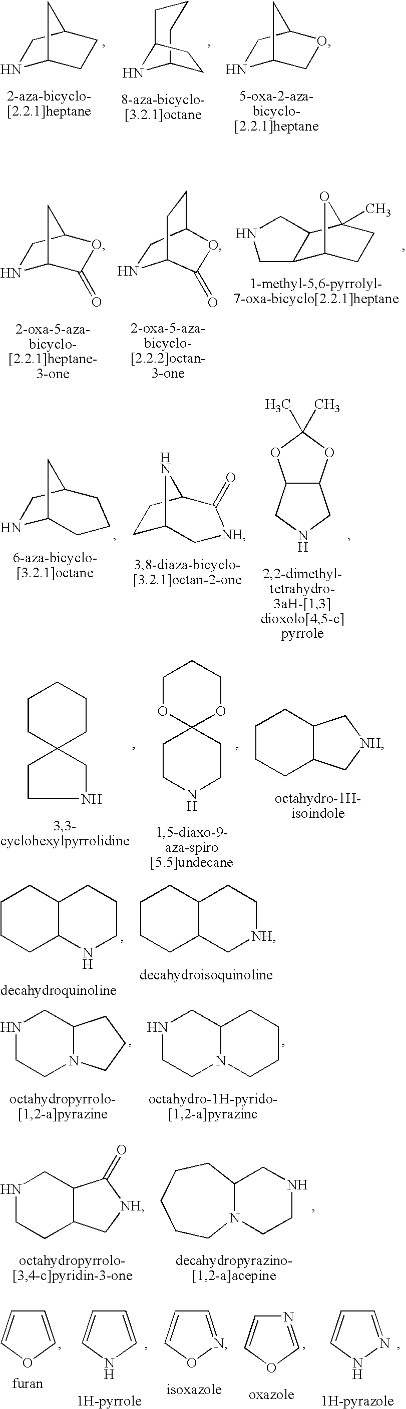

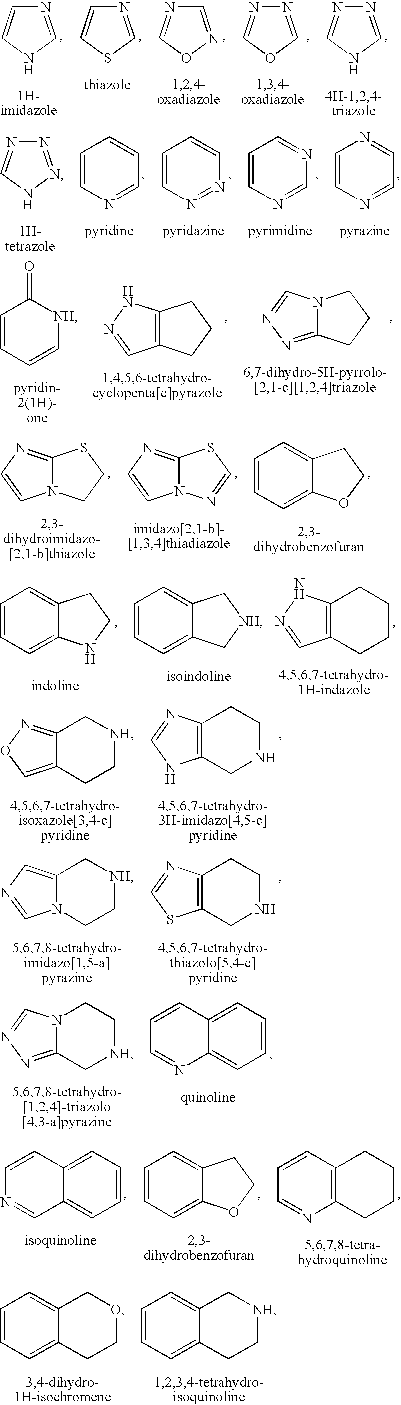

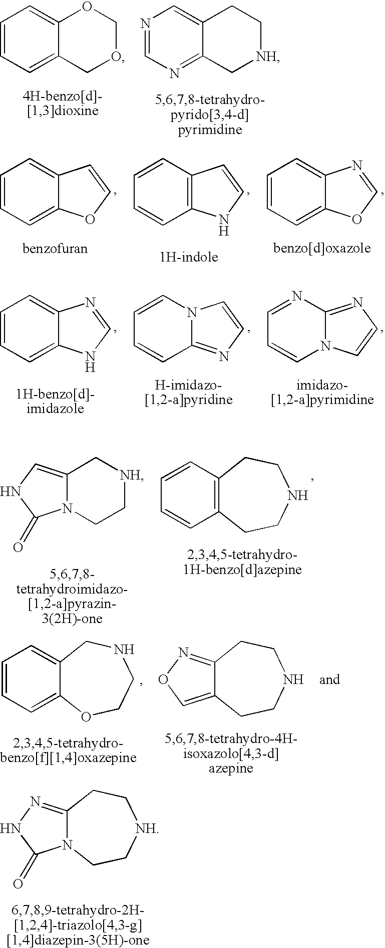

It is to be understood that if a carbocyclic or heterocyclic moiety may be bonded or otherwise attached to a designated substrate, through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

Pharmaceutically acceptable salts of the compounds of the invention include the acid addition and base salts (including disalts) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. For a review on suitable salts, see “Handbook of Pharmaceutical Salts: Properties, Selection, and Use” by Stahl and Wermuth (Wiley-VCH, Weinheim, Germany, 2002).

A pharmaceutically acceptable salt of a compound of the invention may be readily prepared by mixing together solutions of a compound of the invention and the desired acid or base, as appropriate. The salt may precipitate from solution and be collected by filtration or may be recovered by evaporation of the solvent. The degree of ionisation in the salt may vary from completely ionised to almost non-ionised.

The compounds of the invention may exist in both unsolvated and solvated forms. The term ‘solvate’ is used herein to describe a molecular complex comprising a compound of the invention and one or more pharmaceutically acceptable solvent molecules, for example, ethanol, water and the like. The term ‘hydrate’ is included within the meaning of the term “solvate” and is frequently used when the solvent is water. Pharmaceutically acceptable solvates in accordance with the invention include solvates (hydrates) wherein the solvent of crystallization may be isotopically substituted, e.g. D₂O, d₆-acetone, d₆-DMSO.

The compounds of the invention which are complexes, such as clathrates and drug-host inclusion complexes are within the scope of the invention. In contrast to the aforementioned solvates, the drug and host are present in stoichiometric or non-stoichiometric amounts. Also included are complexes containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the invention include all compounds of the invention, polymorphs and isomers thereof, including optical, geometric and tautomeric isomers as hereinafter defined and isotopically-labeled compounds.

The compounds of the invention containing one or more asymmetric carbon atoms may exist as two or more stereoisomers. Where a compound contains an alkenyl or alkenylene group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

All stereoisomers, geometric isomers and tautomeric forms of the compounds of the invention are included within the scope of the invention, including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of the invention contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art [see, for example, “Stereochemistry of Organic Compounds” by E. L. Eliel (Wiley, N.Y., 1994)].

The invention includes all pharmaceutically acceptable isotopically-labelled compounds of the invention, wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁸Cl, fluorine, such as ¹⁸F, iodine, such as ¹²³I and ¹²³I, nitrogen, such as ¹³N and ¹⁶N, oxygen, such as ¹⁵O, ¹⁷O and ¹⁸O, phosphorus, such as ³²P, and sulphur, such as ³⁵S.

Certain isotopically-labelled compounds of the invention, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of the invention can generally be prepared by conventional techniques known to those skilled in the art or by processes analogous to those described in the accompanying Examples and Preparations using an appropriate isotopically-labeled reagents in place of the non-labeled reagent previously employed.

As used herein, the expressions “reaction-inert solvent” and “inert solvent” refers to a solvent which does not interact with starting materials, reagents, intermediates or products in a manner which adversely affects the yield of the desired product.

The parenthetical negative or positive sign used herein in the nomenclature denotes the direction plane polarized light is rotated by the particular stereoisomer.

One of ordinary skill will recognize that certain compounds of the invention may contain one or more atoms which may be in a particular stereochemical or geometric configuration, giving rise to stereoisomers and configurational isomers. All such isomers and mixtures thereof are included in the invention. Solvates (hydrates) of the compounds of the invention are also included.

Other features and advantages will be apparent from the specification and claims which describe the invention.

### DETAILED DESCRIPTION OF THE INVENTION

In general, the compounds of the invention may be prepared by processes known in the chemical arts, particularly in light of the description contained herein. Certain processes for the manufacture of the compounds of the invention are provided as further features of the invention and are illustrated in the reaction schemes provided in the experimental section. The use of various protecting groups in these reactions are also well known and are exemplified in Protective Groups in Organic Synthesis, Second Edition, T. W. Greene and P. G. M. Wuts, John Wiley and Sons, Inc. 1991, pages 227-229, which is hereby incorporated by reference in its entirety for all purposes.

The utility of the compounds of the invention as medical agents for the reduction of intraocular pressure and accordingly to treat glaucoma is demonstrated by the activity of the compounds in conventional assays, including the in vivo assay and a receptor binding assay. Such assays also provide a means whereby the activities of the compounds can be compared to each other and with the activities of other known compounds. The results of these comparisons are useful for determining dosage levels in mammals, including humans, for the treatment of such diseases.

The compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

The compounds of the invention intended for pharmaceutical use may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).]

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium, carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 wt % to 5 wt % of the tablet, and glidants may comprise from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 wt % to 10 wt %, preferably from 0.5 wt % to 3 wt % of the tablet.

Other possible ingredients include anti-oxidants, colourants, flavouring agents, preservatives and taste-masking agents.

Exemplary tablets contain up to about 80% drug, from about 10 wt % to about 90 wt % binder, from about 0 wt % to about 85 wt % diluent, from about 2 wt % to about 10 wt % disintegrant, and from about 0.25 wt % to about 10 wt % lubricant. [Make sure these specific ranges are relevant.]

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verna et al, Pharmaceutical Technology On-line, 25(2), 1-14 (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of the invention used in the preparation of parenteral solutions may be increased by the use of appropriate formulation techniques, such as the incorporation of solubility-enhancing agents.

Formulations for parenteral administration may be formulated to be immediate and/or modified release. Thus, compounds of the invention may be formulated as a solid, semi-solid, or thixotropic liquid for administration as an implanted depot providing modified release of the active compound. Examples of such formulations include drug-coated stents and PGLA [define] microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, J Pharm Sci, 88 (10), 955-958 by Finnin and Morgan (October 1999).]

Other means of topical administration include delivery by electroporation; iontophoresis, phonophoresis, sonophoresis and microneedle or needle-free (e.g. Powderject™, Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). [This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per-actuation and the actuation volume may vary from 1 μl to 100 μl. A typical formulation may comprise a compound of the invention, propylene glycol, sterile-water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from . . . to . . . μg of a compound of the invention. The overall daily dose will typically be in the range . . . μg to . . . mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid; a cellulosic polymer, for example, hydroxypropylmethylcellulose, hydroxyethylcellulose, or methyl cellulose; or a heteropolysaccharide polymer, for example, gelan gum, may be incorporated together with a preservative, such as benzalkonium chloride. Such formulations may also be delivered by iontophoresis.

The compounds of the invention can be incorporated into various types of ophthalmic formulations for delivery to the eye. These compounds may be combined with ophthalmologically acceptable preservatives, surfactants, viscosity enhancers, penetration enhancers, buffers, sodium chloride and water to form an aqueous, sterile ophthalmic suspensions or solutions. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940 or the like according to the published formulations for analogous ophthalmic preparations; preservatives and tonicity agents can be incorporated. Ophthalmic solution formulations may be prepared by dissolving the active ingredient in a physiologically acceptable isotonic aqueous buffer. Further, the ophthalmic solution may include an ophthalmologically acceptable surfactant to assist in dissolving the active ingredient. Furthermore, the ophthalmic solution may contain a thickener such as hydroxymethylcellulose, hydroxyethylcellulose, hydroxypropylmethylcellulose, methylcellulose, polyvinylpyrrolidone, or the like to improve the retention of the medicament in the conjunctival sac.

The compounds of the invention are preferably formulated as topical ophthalmic suspensions or solutions, with a pH of about 4.5 to 7.8. The compounds will normally be contained in these formulations in an amount of 01% to 10% by weight, but preferably in an amount of 0.25% to 5.0% by weight. Thus, for topical presentation 1 to 3 drops of these formulations would be delivered to the surface of the eye 1 to 4 times a day according to the routine discretion of a skilled clinician.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

These dosages are based on an average human subject having a weight of about 65 kg to 70 kg. The physician will readily be able to determine doses for subjects whose weight falls outside this range, such as infants and the elderly. Depending on the [disease and] condition of the patient, the term “treatment” as used herein may include one or more of curative, palliative and prophylactic treatment.

The ability of the compounds of the invention to reduce intraocular pressure may be measured using the assay described below.

The following non-limiting preparations and Examples illustrate the preparation of the compounds of the invention.

### Examples

In the examples described below, unless otherwise indicated, all temperatures are set forth in degrees Celsius and all parts and percentages are by weight. Reagents may be purchased from commercial suppliers, such as Sigma-Aldrich Chemical Company, Acros Organics, or Lancaster Synthesis Ltd. and may be used without further purification unless otherwise indicated. Tetrahydrofuran (THF), methylene chloride (CH₂Cl₂ or DCM), N,N-dimethylacetamide (DMA), acetonitrile (MeCN), and N,N-dimethylformamide (DMF) may be purchased from Aldrich in Sure-Seal bottles and used as received. All solvents may be purified using standard methods known to those skilled in the art, unless otherwise indicated. The ligand bis-(diphenylphosphino)ferrocene is abbreviated as dppf. Diethyl ether is abbreviated as Et₂O. Trifluoroacetic acid is abbreviated as TFA. Acetic acid is abbreviated as HOAc or AcOH. Coupling reagent O-(7-azabenzotriazol-1-yl)-N,N,N′,N′-tetra-methyluronium hexafluorophosphate is abbreviated as HATU.

The reactions set forth below were done generally under a positive pressure of argon or nitrogen or with a drying tube, at ambient temperature (unless otherwise stated), in anhydrous solvents, and the reaction flasks were fitted with rubber septa for the introduction of substrates and reagents via syringe. Glassware was oven dried and/or heat dried. Analytical thin layer chromatography (TLC) was performed using glass-backed silica gel 60 F 254 pre-coated plates (Merck Art 5719) and eluted with appropriate solvent ratios (v/v). Reactions were assayed by TLC or LCMS and terminated as judged by the consumption of starting material. Visualization of the TLC plates was done with UV light (254 nm wavelength) or with an appropriate TLC visualizing solvent and activated with heat. Analytical HPLC performed with Waters or Agilent instruments. Flash column chromatography (Still et al., J. Org. Chem., 1978, 43, 2923) was performed using silica gel 60 (Merck Art 9385) or various MPLC systems, such as Biotage or ISCO purification system. Preparative HPLC routinely performed on Prep LC 4000 system from Water with Ultra 120 10 mm C8 column from Peeke Scientific for single compounds; combinational, solution-based samples described in detail herein. Microwave chemistry was carried out using an Emrys™ Optimizer EXP from Personal Chemistry, Inc. (now Biotage).

The compound structures in the examples below were confirmed by one or more of the following methods: proton magnetic resonance spectroscopy, mass spectroscopy, and elemental microanalysis. Proton magnetic resonance (¹H NMR) spectra were determined using a Bruker spectrometer operating at field strength of 300 or 400 megahertz (MHz). Chemical shifts are reported in parts per million (ppm, δ) downfield from an internal tetramethylsilane standard. Alternatively, ¹H NMR spectra were referenced relative to signals from residual protons in deuterated solvents as follows: CDCl₃=7.25 ppm; DMSO-d₆=2.49 ppm; CD₃CN=1.94 ppm, CD₃OD or methanol-d₄=3.30 ppm; C₆D_{6=7.18} ppm. Peak multiplicities are designated as follows: s, singlet; d, doublet; dd, doublet of doublets; t, triplet; dt, doublet of triplets; q, quartet; br, broadened; m, multiplet. Coupling constants are given in Hertz (Hz). Mass spectra (MS) data were obtained using Agilent LC mass spectrometer with APCI or ESI ionization. High resolution MS (HRMS) were performed on an Agilent G3250AA LCMSD/TOF mass spectrometer. Elemental microanalyses were performed by Atlantic Microlab Inc. and gave results for the elements stated within ±0.4% of the theoretical values.

Preferred compounds in accordance with the invention may be prepared in manners analogous to those specifically described below.

The examples and preparations provided below further illustrate and exemplify the compounds of the present invention and methods of preparing such compounds. It is to be understood that the scope of the present invention is not limited in any way by the scope of the following examples and preparations. The skilled artisan will recognize that different acids, amines, alkyl halides, aryl halides, coupling reagents, and heterocycles may be substituted in the following descriptions to suit the preparations of a desired embodiment. The following methods may be scaled upwards or downwards to suit the amount of desired material.

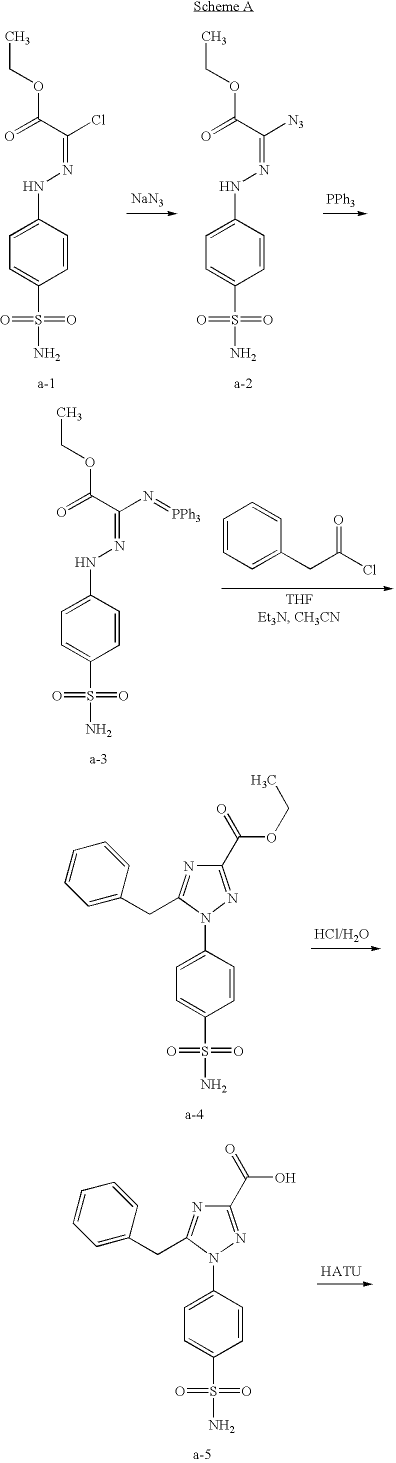

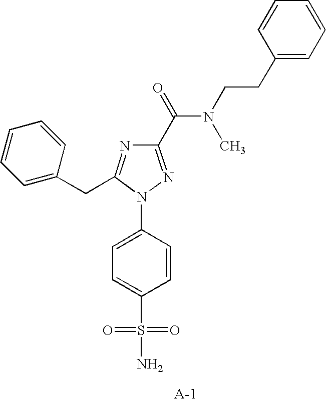

### Method A

### Example A-1

### 1-[4-(Aminosulfonyl)phenyl]-5-benzyl-N-methyl-N-(2-phenylethyl)-1H-1,2,4-triazole-3-carboxamide (A-1)

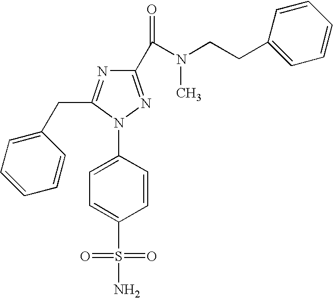

Overall route analogous to the one described by Bruche, L.; et al Synthesis, 1986, 772-774

### Step 1: Ethyl{[4-(Aminosulfonyl)phenyl]hydrazono}(azido)acetate (a-2)

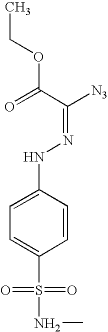

NaN₃ (32.5 g, 0.5 mol), tetrabutylammonium iodide (3.7 g, 0.01 mol), CHCl₃ (300 mL), and H₂O (300 mL) were added to ethyl {[4-(aminosulfonyl)phenyl]hydrazono}-(chloro)acetate (a-1, Cocco, M. T; Farmaco Ed. Sci; 1985; 40; 272-284, 30.6 g, 0.1 mol). The mixture was vigorously stirred overnight. The formed precipitate was separated by filtration and air-dried to constant weight. Compound a-2 (30.0 g) was obtained as a yellow solid, which was used for the next stage without additional purification.

### Step 2: Ethyl{[4-(Aminosulfonyl)phenyl]hydrazono}[triphenylphosphoranylidene)-amino]acetate (a-3)

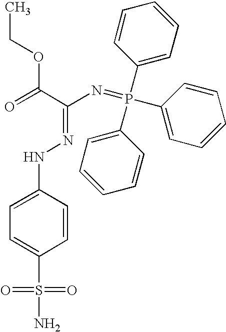

A solution of ethyl{[4-(aminosulfonyl)phenyl]hydrazono}(azido)acetate (a-2, 30.0 g) and triphenylphosphine (25.2 g, 96.1 mmol) in THF (300 mL) was stirred for 2 h. Et₂O (600 mL) was added. The formed precipitate was filtered off, and dried first in air, then in vacuum under heating to give compound a-3 (48.7 g, 89% yield) as a yellow solid, which was used without further purification. ¹H NMR (300 MHz, DMSO-d6) δ 0.92 (t, J=7.1 Hz, 3H) 3.81 (q, J=7.1 Hz, 2H) 7.07 (s, 2H) 7.25 (d, J=8.8 Hz, 2H) 7.54-7.69 (m, 11H) 7.72-7.81 (m, 6H) 9.42 (s, 1H). LCMS: (M+H)⁺: 547.1

### Step 3: Ethyl 1-[4-(Aminosulfonyl)phenyl]-5-benzyl-1H-1,2,4-triazole-3-carboxylate (a-4)

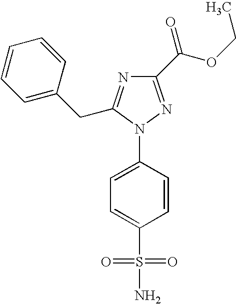

Ethyl-{[4-(aminosulfonyl)phenyl]hydrazono}[(triphenylphosphoranylidene)amino]acetate (a-3, 15.8 g, 0.029 mol) was dissolved in THF (300 mL). Benzoyl chloride (11.5 mL, 0.087 mol) was added, and the mixture was stirred overnight. After this, Et₂O (1.1 L) was added. The formed precipitate was separated by filtration, washed with Et₂O (100 mL), and air-dried to give an orange solid. This substance was dissolved in acetonitrile (300 mL). Triethylamine (24 mL, 0.174 mol) was added. The mixture was stirred overnight and evaporated. The residue was purified by chromatography on a silica gel column (CH₂Cl₂/ethyl acetate 1:1) to give compound a-4 (6.5 g, 58% yield) as a white solid. NMR provided in table. Elemental Analysis: Calcd for C₁₈H₁₈N₄O₄S-0.2 hexane: 57.13; H, 5.19; N, 13.88. Found: C, 57.25; H, 5.34; N, 13.89.

### Step 4: 1-[4-(Aminosulfonyl)phenyl]-5-benzyl-1H-1,2,4-triazole-3-carboxylic acid (a-5)

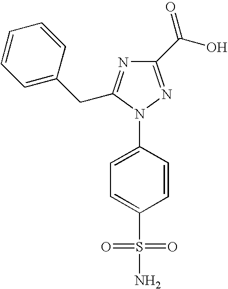

Ethyl 1-[4-(aminosulfonyl)phenyl]-5-benzyl-1H-1,2,4-triazole-3-carboxylate a-4 (6.5 g, 0.017 mol) was refluxed for 2 it in 6N HCl (80 mL). The solvent was evaporated, and water (30 mL) was poured to the residue. The solution was made alkalized with KOH to pH 10-11. The obtained water solution was extracted with dichloromethane (2×25 mL) and ethyl acetate (4×25 mL). Then the water solution was acidified to pH 2-3 and cooled to 0° C. The formed precipitate was separated by filtration and dried initially in air and then over P₂O₆ in a desiccator to give compound a-5 (4.79 g, 79% yield) as a beige solid, which was used without further purification. NMR provided in table. Elemental Analysis: Calcd for C₁₆H₁₄N₄O₄S.1.0 H₂O: C, 51.06; H, 4.28; N, 14.89. Found: C, 51.09; H, 4.26; N, 14.98.

### Step 5: 1-[4-(Aminosulfonyl)phenyl]-5-benzyl-N-methyl-N-(2-phenylethyl)-1H-1,2,4-triazole-3-carboxamide (A-1)

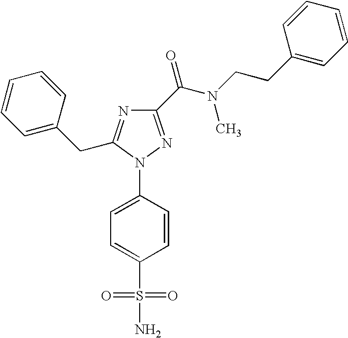

1-[4-(Aminosulfonyl)phenyl]-5-benzyl-1H-1,2,4-triazole-3-carboxylic acid a-5 (1.0 g, 2.79 mmol) in anhydrous DMF (8 mL) was added O-(7-Azabenzotriazole-1-yl)-N, N,N′N′-tetramethyluronium hexafluorophosphate (HATU, 1.27 g, 3.35 mmol), followed by the addition of triethylamine (0.47 mL, 3.35 mmol) and N-methylphenethylamlne (0.49 mL, 3.35 mmol). The mixture was stirred at room temperature for 5 hours and purified by preparative HPLC using CH₃CN and water, the product (1.19 g) was obtained as a white powder, yield 90%. NMR provided in table. Elemental Analysis: Calcd for C₂₅H₂₅N₃O₃S.0.5 TFA: C, 58.64; H, 4.83; N, 13.15. Found: C, 58.55; H, 5.04; N, 13.46.

Examples a-4a to a-4z were prepared from the appropriate starting material in a manner analogous to the method of Example a-4.

Examples a-5a to a-5c were prepared from the appropriate starting material in a manner analogous to the method of Example a-5.

Examples A-1a to A-1e were prepared from the appropriate starting material in a manner analogous to the method of Example A-1.

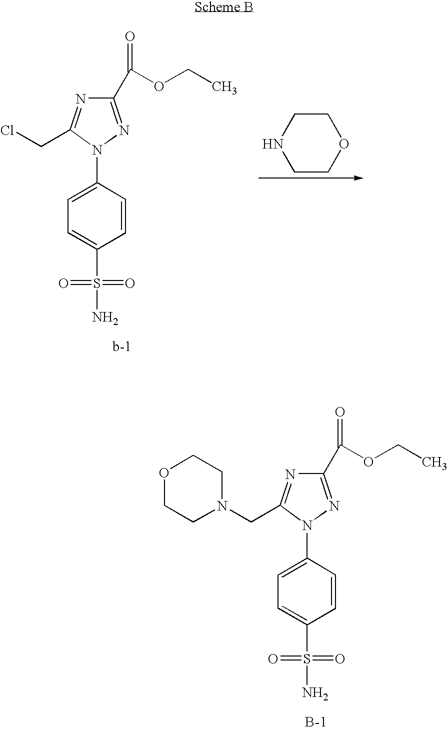

### Method B

### Example B-1

### Ethyl 1-[4-(Aminosulfonyl)phenyl]-5-(morpholin-4-ylmethyl)-1H-1,2,4-triazole-3-carboxylate (B-1)

Ethyl 1-[4-(aminosulfonyl)phenyl]-5-(chloromethyl)-1H-1,2,4-triazole-3-carboxylate b-1 (200 mg, 0.58 mmol, made according to the procedure for Compound a-4 from chloroacetyl chloride) in 2 mL of acetonitrile was added morpholine (0.20 mL, 2.3 mmol). The mixture was stirred for 20 minutes. The solvent was evaporated, the residue was diluted with 1:1 ether and hexane, the suspension was stirred rapidly for 5 minutes, filtered. The solid was purified by preparative HPLC using CH₃CN and water to give the title compound as a white powder (158 mg, Yield 69%). NMR provided in Table. Elemental Analysis: Calcd for C₁₆H₂₁N₅O₅S.1.5 HCl: C, 42.69; H, 5.04; N, 15.56. Found: C, 42.91; H, 5.08; N, 15.38.

Examples B-1a was prepared from the appropriate starting material in a manner analogous to the method of Example B-1.

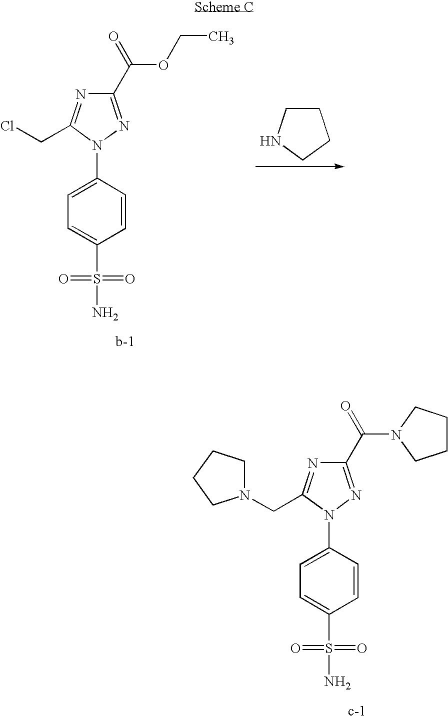

### Method C

### Example C -1

### 4-[3-(Pyrrolidin-1-ylcarbonyl)-5-(pyrrolidin-1-ylmethyl)-1H-1,2,4-triazol-1-yl)benzenesulfonamide (C-1)

The title compound was made in similar fashion as Example B-1, except the reaction time was one hour to achieve 90% yield. NMR provided in Table. Elemental Analysis: Calcd for C₁₈H₂₄N₆O₃S.2.4 TFA C, 40.38; H, 3.92; N, 12.39. Found: C, 40.54; H, 4.10; N, 12.23.

Examples C-1a was prepared from the appropriate starting material in a manner analogous to the method of Example C-1.

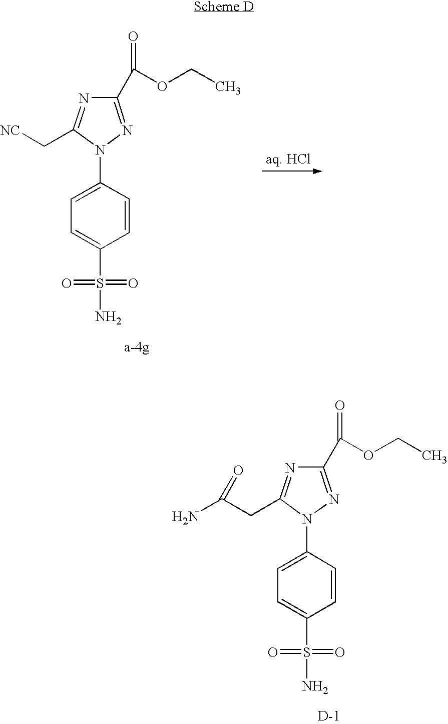

### Method D

### Example D-1

### Ethyl 5-(2-amino-2-oxoethyl)-1-[4-(aminosulfonyl)phenyl]-1H-1,2,4-triazole-3-carboxylate (D-1)

Ethyl 1-[4-(aminosulfonyl)phenyl]-5-(cyanomethyl)-1H-1,2,4-triazole-3-carboxylate a-4g (200 mg, 0.60 mmol, made according to the procedure from Compound a-4) was stirred in the mixture of 6N HCl (5 mL) and EtOH (5 mL) overnight. The solvent was evaporated. The solid was filtered and purified by preparative HPLC using CH₃CN and water to give the title compound as a white powder (89 mg, yield 42%), which was used without further purification. NMR provided in table. Elemental Analysis: Calcd for C₁₅H₁₅N₅O₆S: C, 44.19; H, 4.28; N, 19.82. Found: C, 44.31; H, 4.35; N, 19.98.

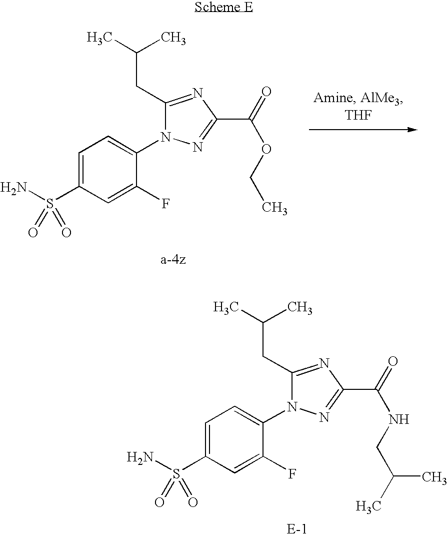

### Method E

### Example E-1

### 1-[4-(Aminosulfonyl)-2-fluorophenyl]-N,5-diisobutyl-1H-1,2,4-triazole-3-carboxamide (E-1)

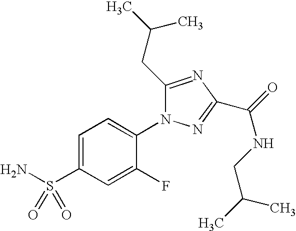

To a cooled to 0° C. solution of 2-methylpropan-1-amine (0.89 g, 1.76 mmol) in tetrahydrofuran (1 mL) was slowly added trimethylaluminum 2M in toluene (0.316 mL, 0.63 mmol). The mixture was stirred at room temperature for 2 h. The solution was slowly added to ethyl 1-[4-(aminosulfonyl)-2-fluorophenyl]-5-isobutyl-1H-1,2,4-triazole-3-carboxylate (a-4z, 0.086 g, 0.23 mmol) in tetrahydrofuran (1 mL) and the mixture was stirred at 65° C. for 18 h. The mixture was quenched with MeOH and 1N hydrochloric acid then partially concentrated. The residue was purified by reverse phase HPLC using acetonitrile in water (5 to 95%, with 0.1% trifluoroacetic acid). The resulting material was dissolved in methanol and 3N hydrochloride in methanol and the mixture stirred at room temperature for 5 minutes. The mixture was then concentrated to dryness, suspended in toluene and concentrated to dryness to give the hydrochloride salt of the title compound E-1 (0.081 g, 86%). NMR provided in table. Elemental Analysis: Calcd for C₁₇H₂₄FN₅O₃S-0.3 HCl-0.8 H₂O C, 48.29; H, 6.17; N, 16.56. Found: C, 48.19; H, 6.08; N, 16.30.

Examples E-1a and E-1b were prepared from the appropriate starting material in a manner analogous to the method of Example E-1.

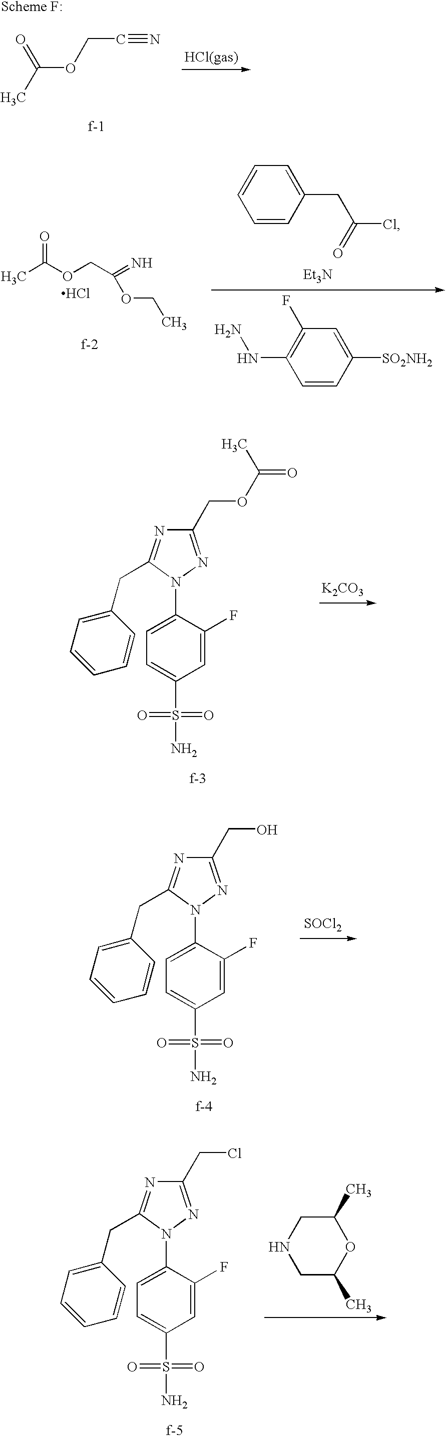

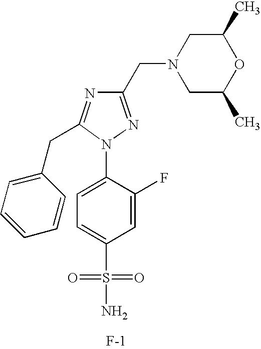

### Method F

### Example F-1

### 4-(5-Benzyl-3-{[2,6-cis-dimethylmorpholin-4-yl]methyl}-1H-1,2,4-triazol-1-yl)-3-fluorobenzenesulfonamide (F-1)

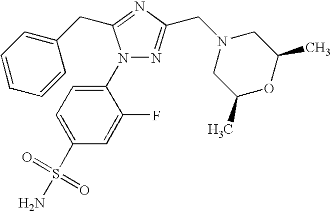

### Step 1: 2-Ethoxy-2-Iminoethyl Acetate Hydrochloride Salt (f-2)

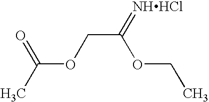

Into a solution of cyanomethyl acetate (f-1, 14.86 g, 150 mmol, Wagenknecht, J. H.; et al Synthetic Communication, 1972, 2, 215-219) and anhydrous ethanol (6.91 g, 150 mmol) in anhydrous diethyl ether (69 mL), HCl gas was introduced and bubbled at 0° C. for 15 minutes. After a white solid precipitated, the reaction mixture was stirred continuously at 0° C. for 30 minutes. Ether (70 ml) was added to suspend the salt. Filtration and collection gave a white solid. It was dried under reduced pressure at room temperature to afford 2-ethoxy-2-iminoethyl acetate hydrogen chloride salt as a white solid (23.6 g, 86%), which was used without further purification. ¹H NMR (400 MHz, DMSO-d₆) δ ppm 1.35 (t, J=6.9 Hz, 3H) 2.17 (s, 3H) 4.52 (q, J=6.9 Hz, 2H) 4.97 (s, 2H)

### Step 2: {1-[4-(Aminosulfonyl)-2-fluorophenyl]-5-benzyl-1H-1,2,4-triazol-3-yl)methyl Acetate (f-3)

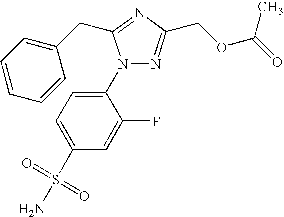

Procedure 1: A 500 mL three-necked flask equipped with a thermometer and a dropping funnel was charged with 2-ethoxy-2-iminoethyl acetate hydrochloride (f-2, 20.0 g, 0.110 mol) and dichloromethane (200 mL). The reaction mixture was cooled to 0° C., and triethylamine (30.3 g, 0.3 mol) was added dropwise under vigorous stirring in a flow of argon. The mixture was stirred at the same temperature for 30 min, and then a solution of phenylacetyl chloride (18.5 g, 0.120 mol) in dichloromethane (50 mL) was added. The reaction mixture was stirred for 2 h at 0° C., and then 3-fluoro-4-hydrazinobenzene-sulfonamide (20.5 g, 0.1 mol, PaI, M. J.; et at J. Med. Chem. 2003, 46; 3975-3984) was added in portions. The cooling bath was removed. The reaction mixture was heated to room temperature, refluxed for 1 h, and rotary-evaporated. The residue was dissolved in absolute THF (200 mL). Acetic acid (50 mL) was added, and the solution was refluxed for 2 h under TLC monitoring. The reaction mass was rotary-evaporated to dryness. The residue was diluted with water, and the product was extracted with chloroform. The extract was dried with Na₂SO₄, dried, and evaporated. The residue was purified by chromatography (silica gel, dichloromethane/methanol (100:0→80:20) to give the title compound f-3 (27.6 g, 68%), which was used without further purification.

Procedure 2: Triethylamine (3.53 mL, 25.3 mmol) was added into the suspension of 2-ethoxy-2-iminoethyl acetate hydrochloride (f-2) in anhydrous THF (20 mL) at 0° C. under nitrogen, then a solution of phenylacetyl chloride (1.46 mL, 11.0 mmol) in THF (10 mL) was added into the mixture dropwise. The mixture was stirred at 0° C. for 2 hours. 3-Fluoro-4-hydrazinobenzene-sulfonamide (2.25 g, 11.0 mmol) was added along with THF (20 mL), the mixture was heated at 60° C. for ‘6 hours, and allowed to cool. The solvent was evaporated, and the residue was dissolved in 10% MeOH/CHCl₃ (300 mL). The solution was adjusted to pH6 with 1N HCl and washed with brine, the organic layer was dried over Na₂SO₄ and concentrated to give the title product (2.76 g) in yellow hard foam which was used into the next step without further purification. LCMS (M+H)⁺: 405.2.

### Step 3: 4-[5-Benzyl-3-(hydroxymethyl)-1H-1,2,4-triazol-1-yl)-3-fluorobenzenesulfonamide (f-4)

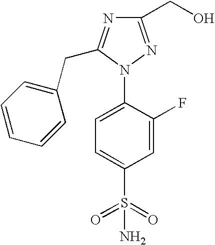

K₂CO₃ (9.4 g, 0.068 mol) was added to a mixture of (1-[4-(aminosulfonyl)-2-fluorophenyl]-5-benzyl-1H-1,2,4-triazol-3-yl}methyl acetate (f-3; 13.8 g, 0.034 mol) and methanol (100 mL). The reaction mixture was stirred at room temperature for 1 h (TLC monitoring). The reaction mass was evaporated by one half of the volume and cooled to room temperature. The formed precipitate was separated by filtration, washed with ether, and dissolved in water. The solution was acidified with citric acid. The formed precipitate was separated by filtration, washed several times with water on the filter, and dried under vacuum in a rotary evaporator to afford the title compound (8.1 g, 65%). NMR provided in table.

### Step 4: 4-[5-Benzyl-3-(chloromethyl)-1H-1,2,4-triazol-1-yl]-3-fluorobenzene-sulfonamide (f-5)

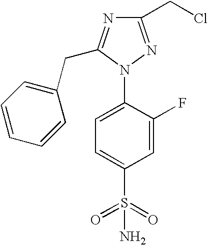

To 4-[5-benzyl-3-(hydroxymethyl)-1H-1,2,4-triazol-1-yl]-3-fluorobenzenesulfonamide f-4 (1.2 g, 3.3 mmol) in a flask under nitrogen was added thionyl chloride (10 mL). The mixture stirred for 2 hr at ambient temperature. Thionyl chloride was evaporated, the residue was azeotroped with heptane (5 mL) three times to give the title compound as a yellow powder, which was used without further purification. ¹H NMR (400 MHz, DMSO-d₆) 4.11 (s, 2H) 4.78 (s, 2H) 7.06 (d, J=6.80 Hz, 2H) 7.11-7.28 (m, 3H) 7.73 (s, 2H) 7.76-7.94 (m, 3H). LCMS (M+H)⁺: 381

### Step 5: 4-(5-Benzyl-3-{[2,6-cis-dimethylmorpholin-4-yl]methyl}-1H-1,2,4-triazol-1-yl)-3-fluorobenzenesulfonamide (F-1)

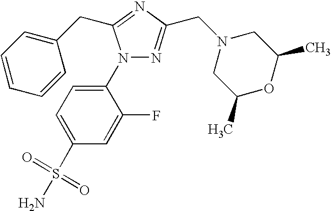

4-[5-Benzyl-3-(chloromethyl)-1H-1,2,4-triazol-1-yl]-3-fluorobenzenesulfonamide (f-5; 200 mg, 0.53 mmol) in anhydrous DMSO (2 mL) was added 2,6-cis-dimethylmorpholin (2.63 mmol). The mixture was stirred for 2 hours. The title compound C-1 (150 mg, 62% for two steps) was obtained as a white powder after the purification by reverse phase HPLC using acetonitrile in water (5 to 95%, with 0.1% trifluoroacetic acid). NMR provided in Table. Elemental Analysis: Calcd for C₂₂H₂₆FN₅O₃S.1.0 HCl: C, 53.27; H, 5.49; N, 14.12. Found: C, 53.35; H, 5.56; N, 14.08.

Examples f-3a to f-3k were prepared from the appropriate starting material in a manner analogous to the method of Compound f-3 (Method F, step 2).

Examples f-4a to f-4p were prepared from the appropriate starting material in a manner analogous to the method of Example f-4.

Examples F-1a to F-1z were prepared from the appropriate starting material in a manner analogous to the method of Example F-1.

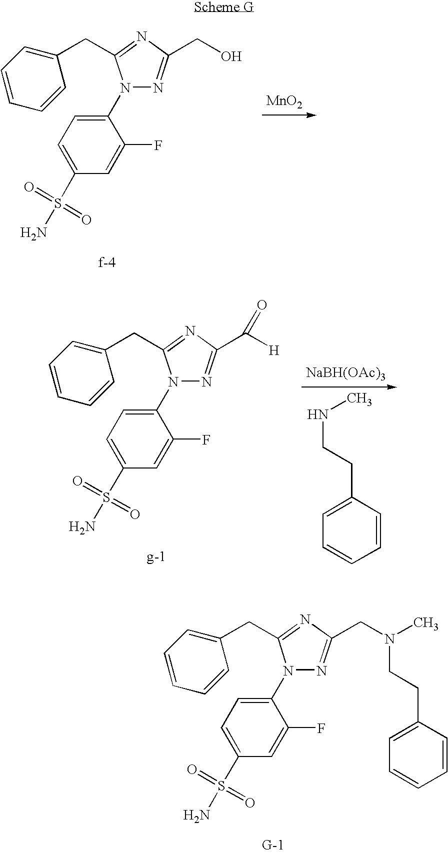

### Method G

### Example G-1

### Step 1: 4-(5-Benzyl-3-formyl-1H-1,2,4-triazol-1-yl)-3-fluorobenzenesulfonamide (g-1)

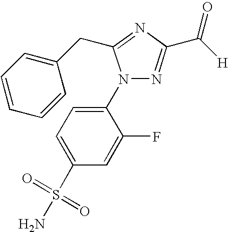

A 250 mL flask was charged with alcohol f-4 (8.0 g, 0.022 mol) and acetone (50 mL). MnO₂ (0.22 mol, 19.2 g) was added in portions at room temperature in a flow of argon under vigorous stirring for 4 h (TLC monitoring). The reaction mixture was filtered through a thin layer of Celite, and the solid on the filter was washed many times with acetone. The filtrate was evaporated under vacuum at 28° C. to give the title compound g-1 (4.23 g, 53%, >90% purity by HPLC and NMR). Typically used without further purification. 1H NMR (400 MHz, Acetone-d₆) 4.25 (s, 2H), 7.07-7.15 (m, 2H), 7.17-7.28 (m, 3H), 7.73-7.80 (m, 2H), 7.81-7.87 (m, J=1.76 Hz, 1H), 9.98 (s, 1H).

### Step 2: 4-(5-Benzyl-3-{[methyl(2-phenylethyl)amino]methyl}-1H-1,2,4-triazol-1-yl)-3-fluorobenzene-sulfonamide (G-1)

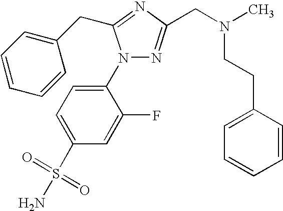

The title compound was made in similar procedure as described in Example PP-1, step 5. 4-(5-benzyl-3-formyl-1H-1,2,4-triazol-1-yl)-3-fluorobenzenesulfonamide g-1 and N-methyl-2-phenylethanamine gave the title compound in 83% yield.

Examples G-1a to G-1k were prepared from the appropriate starting material in a manner analogous to the method of Example G-1. NMR and MS provided in Table.

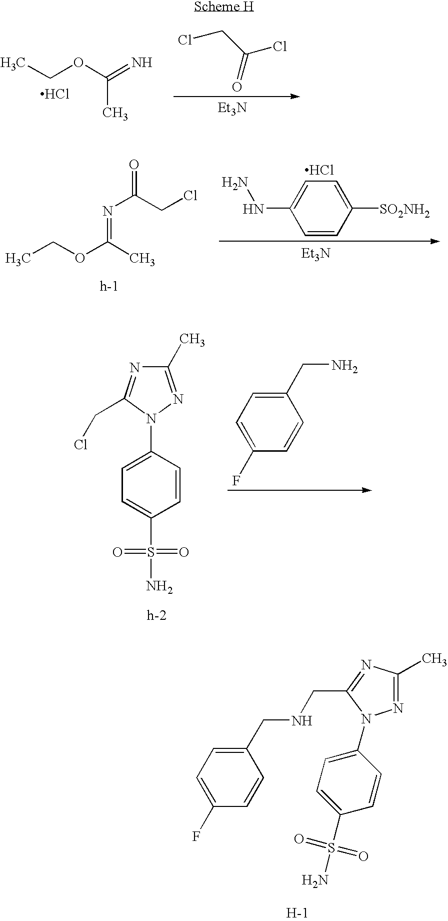

### Method H

### Example H-1

### 4-(5-{[(4-Fluorobenzyl)amino]methyl}-3-methyl-1H-1,2,4-triazol-1-yl)benzenesulfonamide (H-1)

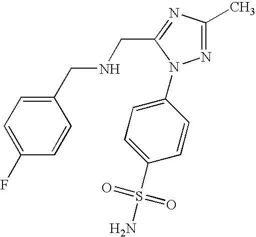

### Step 1: Ethyl N-2-Chloroacetimidate (h-1)

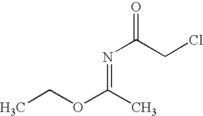

The title compound was made according to the procedure from Bayard, P. Tetrahedron Letters, 1988, 29, 3799-3802. Triethylamine (24.8 mL, 178 mmol) was added to a vigorously stirred slurry of ethyl acetimidate hydrochloride (10.0 g, 80.9 mmol) in dry CH₂Cl₂ at −35° C., then chloroacetyl chloride (6.44 mL, 80.9 mmol) was added rapidly. The cooling bath was removed. The mixture was stirred for 30 minutes, petroleum ether (200 mL) was added in one portion. The mixture was filtered off and the filtrate was concentrated in vacuo. The residue was again dissolved in petroleum ether. Any insoluble material removed by filtration, the solution was concentrated. The resultant yellow oil was typically used without further purification. ¹H NMR (300 MHz, chloroform-d) 1.31 (t, J=7.2 Hz, 3H) 2.10 (s, 3H) 4.15 (s, 2H) 4.16 (q, J=7.2 Hz, 3H)

### Step 2: 4-[5-(Chloromethyl)-3-methyl-1H-1,2,4-triazol-1-yl]-3-fluorobenzenesulfonamide (h-2)

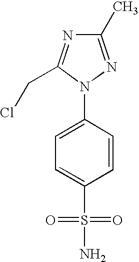

2-Chloro-N-[(1E)-2-methoxy-1-methylethylidene]acetamide (h-1, 250 mg, 1.94 mmol) and 4-hydrazinobenezene-1-sulfonamide HCl salt (361 mg, 1.61 mmol) was suspended in dry THF (5 mL), then triethylamine (0.50 mL, 355 mmol) was added. The mixture was stirred for overnight. The solvent was removed. The residue was extracted with 10% CH₃OH/CHCl₃ and brine. The organic layer was dried over dry Na₂SO₄ and concentrated. The resultant yellow oil was taken upon into 2 mL of methanol and stood for overnight. The yellow precipitate was washed with small amount of ether and dried under vacuum to give the title compound as a yellow powder (305 mg, yield 55%), which was used without further purification. LCMS (M+H)⁺: 287.1

### Step 3: 4-(5-{[(4-Fluorobenzyl)amino]methyl}-3-methyl-1H-1,2,4-triazol-1-yl)benzenesulfonamide (H-1)

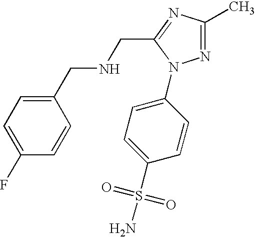

4-[5-(Chloromethyl)-3-methyl-1H-1,2,4-triazol-1-yl]-3-fluorobenzenesulfonamide (h-2, 200 mg, 0.70 mmol) was suspended in CH₃CN (3 mL), and 4-fluorobenzylamine (0.24 mL, 2.1 mmol) was added. The solution was stirred overnight. The solvent was removed, the residue was dissolved in 1 mL of MeOH and diluted with 20 mL of 1:1 ether/hexane with rapid stirring. The precipitate was filtered and washed with small amount of water and dried under vacuum to give the title compound as a white powder (190 mg, yield 69%). NMR provided in Table. Elemental Analysis: Calcd for C₁₇H₁₈FN₅O₂S: C, 54.39; H,.4.83; N, 18.65. Found: C, 54.35; H, 4.87; N, 18.56.

Examples H-1a to H-1x were prepared from the appropriate starting material in a manner analogous to the method of Example H-1.

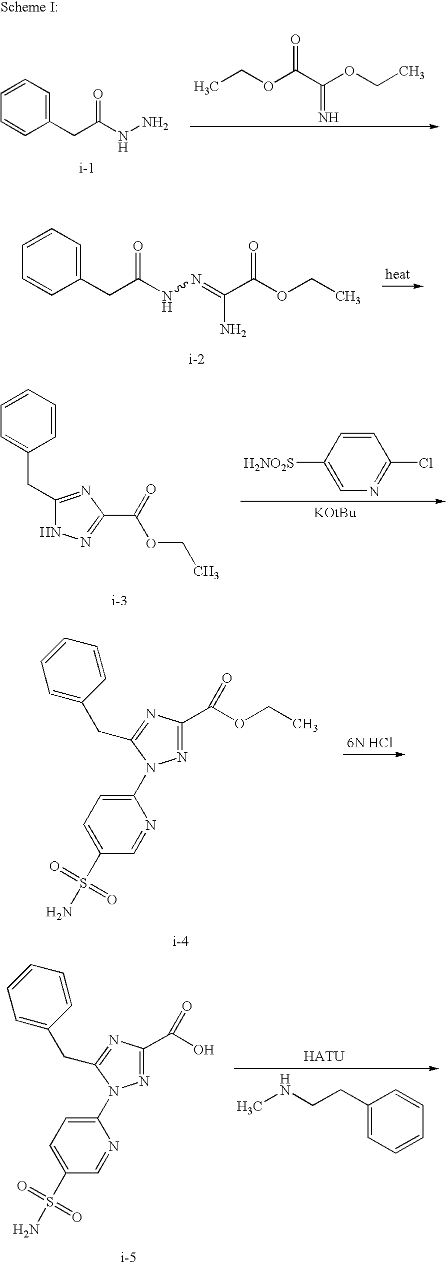

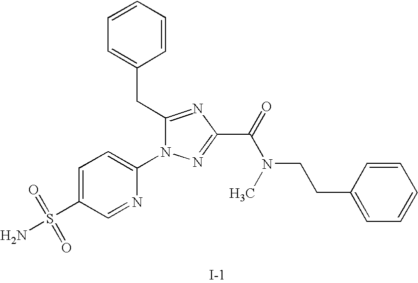

### Method I

### Example I-1

### 1-[5-(Aminosulfonyl)pyridin-2-yl]-5-benzyl-N-methyl-N-(2-phenylethyl)-1H-1,2,4-triazole-3-carboxamide (I-1)

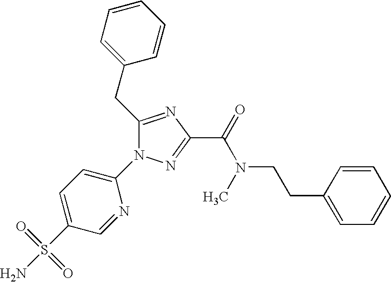

### Step 1: Ethyl-2-Amino[(phenylacetyl)hydrazono]acetate (i-2)

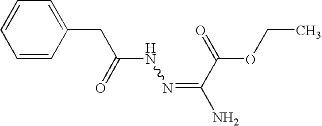

Adapting a route similar to that described in Catarzi, D.; et al J. Med. Chem.; 1995; 38; 2196-2201; 2-phenyl-acetohydrazide (I-1, 6.92 g, 46.1 mmol, Prata, J. V; J. Chem. Soc. Perkin trans.1; 2002; 513-528) and ethyl ethoxy(imino)acetate (6.69 g, 46.1 mmol, McKillop, A.; Synthesis; 1997; 3; 301-304) in EtOH (60 mL) was stirred under nitrogen at ambient temperature for overnight. The resultant suspension was filtered. The white solid was washed with EtOH and dried under vacuum to give the title compound i-2 (9.0 g, yield 78%) which was used without further purification. ¹H NMR (300 MHz, DMSO-d₆) mixture of isomers 3.51 (s, 0.56H, major isomer) 3.87 (s, 0.44H, minor isomer) 6.44 (s, 0.88H, minor isomer) 6.53 (s, 1.12H, major isomer) 9.93 (s, 1.12H, major isomer) 9.98 (s, 0.88H, minor isomer)

### Step 2: Ethyl 5-Benzyl-1H-1,2,4-triazole-3-carboxylate (i-3)

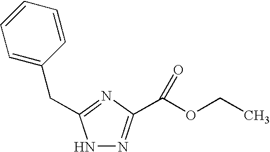

Adapting a procedure similar to that described in Catarzi, D.; et al J. Med. Chem.; 38; 1995; 2196-2201, neat methyl 2-amino[(phenylacetyl)hydrazono]acetate (i-2, 6.5 g, 26.1 mmol) in a flask was placed in a pre-heated oil bath at 200° C. for 15 minutes. The melt was allowed to cool, the resultant solid taken up into MeOH (30 mL), and then the solvent was evaporated. The resultant white solid was suspended in ether (60 mL), stirred for 10 minutes, filtered off, washed with ether, and dried under vacuum to give the title compound i-3 (3.1 mg, yield 49%), which was used without further purification. ¹H NMR (300 MHz, DMSO-d₆) 1.30 (t, J=7.2 Hz, 3H) 4.14 (s, 2H) 4.31 (q, J=7.2 Hz, 2H) 7.23-7.38 (m, 5H) 14.50 (s, 1H). LCMS (M+H)⁺: 232

### Step 3: Ethyl 1-[5-(Aminosulfonyl)pyridin-2-yl]-5-benzyl-1H-1,2,4-triazole-3-carboxylate (i-4)

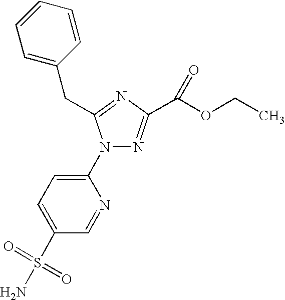

To the mixture of ethyl 5-benzyl-1H-1,2,4-triazole-3-carboxylate (i-3, 1.16 g, 5.00 mmol) and 6-chloropyridine-3-sulfonamide (1.73 g, 9.00 mmol, Adams; J. Am. Chem. Soc. 71, 1949, 387-389) in DMSO (10 mL) was added potassium t-butoxide (842 mg, 7.50 mmol). The mixture was heated in a microwave apparatus at 120° C. for 90 minutes. TLC showed some of the starting material left. The solution was heated at 120° C. in microwave for another 90 minutes, cooled. The mixture was diluted with water (200 mL) and 1N HCl (5 mL), decanted. The sticky residue was suspended into MeOH (5 mL), then diluted with water (200 mL), the mixture was stirred rapidly for half hour, filtered. The yellow solid was washed with water and dried under vacuum to give the title compound i-4 (860 mg, yield 44%), which was used without further purification. Regiochemistry assumed from a similar experiment described for pyrazole Example mm-1. NMR provided in table. Elemental Analysis: Calcd for C₁₇H₁₇N₆O₄S; C, 52.70; H, 4.42; N, 18.08. Found: C, 52.57; H, 4.45; N, 18.00.

### Step 4: 1-[5-(Aminosulfonyl)pyridin-2-yl]-5-benzyl-1H-1,2,4-triazole-3-carboxylic Acid (i-5)

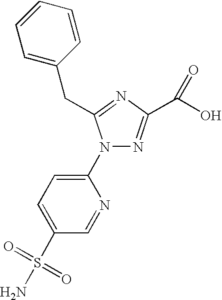

A suspension of ethyl 1-[5-(aminosulfonyl)pyridin-2-yl]-5-benzyl-1H-1,2,4-triazole-3-carboxylate (i-4, 860 mg, 2.22 mmol) in 20 mL of conc. HCl and 20 mL of water was heated at reflux for 1 hour. The solvent was evaporated. The residue was purified by reverse phase HPLC using acetonitrile in water to give the title compound i-5 as a white solid (710 mg, yield 89%). Typically the acid i-5 used without further purification. NMR provided in Table. Elemental Analysis: Calcd for C₁₅H₁₃N₅O₄S.0.6 HCl: C, 47.26; H, 3.60; N, 18.37. Found: C, 47.41; H, 3.69; N, 18.48.

### Step 5: 1-[5-(Aminosulfonyl)pyridin-2-yl]-5-benzyl-N-methyl-N-(2-phenylethyl)-1H-1,2,4-triazole-3-carboxamide (i-1)

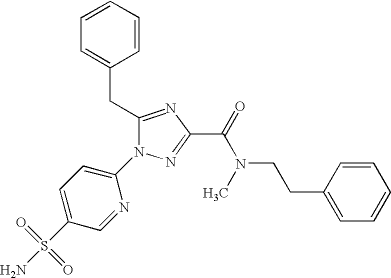

The title compound was obtained in a similar manner as described for Example A-1, step 5. 1-[5-(Aminosulfonyl)pyridin-2-yl]-5-benzyl-1H-1,2,4-triazole-3-carboxylic acid (i-5, 200 mg, 0.56 mmol) and N-methylphenylethylamine (92 mg, 0.68 mmol) gave the title compound E-1 as a white powder (234 mg, 88% yield). NMR provided in Table. Elemental Analysis: Calcd for C₂₄H₂₄N₆O₃S.0.2 TFA: C, 58.69; H, 4.88; N, 16.83. Found: C, 58.99; H, 5.03; N, 16.77.

Example I-1a was prepared from the appropriate starting material in a manner analogous to the method of Example i-1.

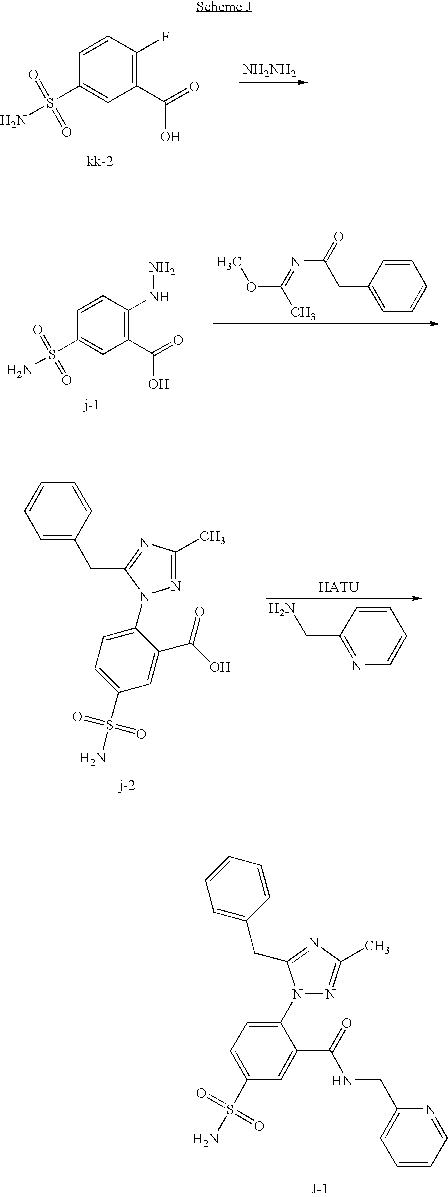

### Method J

### Example J-1

### 5-(Aminosulfonyl)-2-(5-benzyl-3-methyl-1H-1,2A-triazol-1-yl)-N-(pyridin-2-ylmethyl)benzamide (J-1)

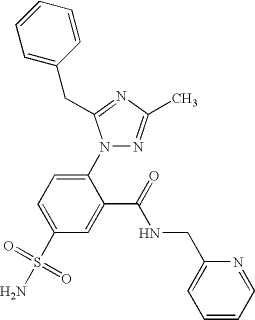

### Step 1: 5-(Aminosulfonyl)-2-hydrazinobenzoic Acid (j-1)

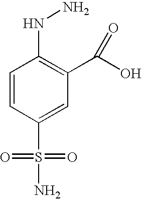

5-(Aminosulfonyl)-2-fluorobenzoic acid (kk-2, 10.0 g, 45.6 mmol) was suspended in EtOH (23 mL) and water (3 mL), NH₂NH₂.H₂O (11 mL, 228 mmol) was added. The mixture was stirred at 60° C. for four hours, the suspension became a solution. The solvent was evaporated, the residue was cooled to 0° C. and acidified to pH 3 using 6N HCl. The resultant precipitate was filtered and washed with water, then dried under vacuum. The title compound (9.55 g, 90%) was obtained as a yellow powder, which was used without further purification. ¹H NMR (300 MHz, DMSO-d₆) δ 7.07 (s, 2H) 7.39 (d, J=9.0 Hz, 1H) 7.74 (dd, J=9.0, 2.3 Hz, 1H) 8.21 (d, J=2.3 Hz, 1H)

### Step 2: 5-(Aminosulfonyl)-2-(5-benzyl-3-methyl-1H-1,2,4-triazol-1-yl)benzoic Acid (j-2)

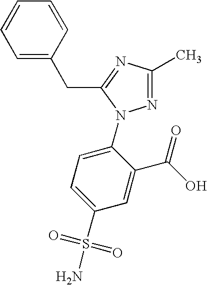

The title compound was made in the similar fashion as described in for Example H-1, step 2, except the reaction temperature was 60° C. 5-(Aminosulfonyl)-2-hydrazinobenzoic acid (j-1, 2.80 g, 12.1 mmol) and N-[(1E)-2-methoxy-1-methylethylidene]-2-phenylacetamide (2.73 g, 13.3 mmol, made according to the procedure for Compound h-1) gave the title compound as a yellow solid (3.8 g, yield 84%). NMR provided in Table. Elemental Analysis: Calcd for C₁₇H₁₈N₄O₄S: C, 54.83; H, 4.33; N, 15.04. Found: C, 54.59; H, 4.32; N, 14.93.

### Step 2: 5-(Aminosulfonyl)-2-(5-benzyl-3-methyl-1H-1,2,4-triazol-1-yl)-N-(pyridin-2-ylmethyl)benzamide (J-1)

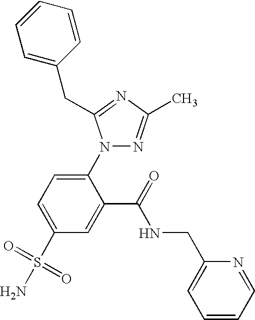

The title compound was made in similar fashion as described in Example A1, Step 5. 5-(Aminosulfonyl)-2-(5-benzyl-3-methyl-1H-1,2,4-triazol-1-yl)benzoic acid (j-2, 250 mg, 0.67 mmol) and 2-(aminomethyl)pyridine (0.076 mL, 0.74 mmol) gave the title compound as a yellow powder (201 mg, yield 65%). NMR provided in Table. Elemental Analysis: Calcd for C₂₃H₂₂N₈O₃S.3.0 HCl.2.0 H₂O C, 45.44; H, 4.81; N, 13.82. Found: C, 45.31; H, 4.65; N, 13.61.

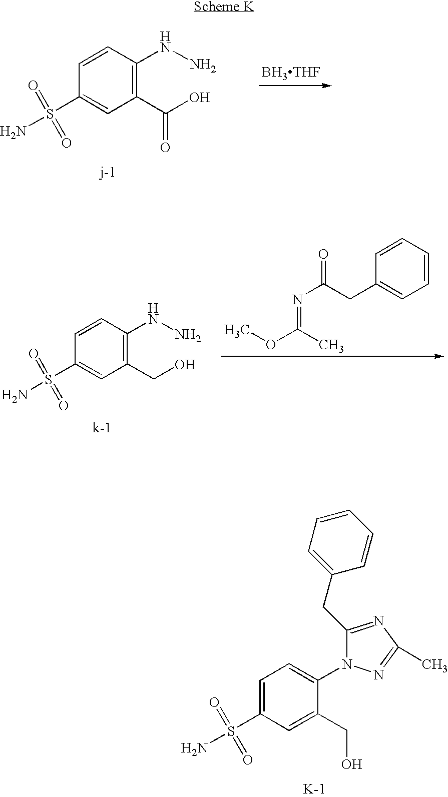

### Method K

### Example K-1

### 4-(5-Benzyl-3-methyl-1H-1,2,4-triazol-1-yl)-3-(hydroxymethyl)benzenesulfonamide (K-1)

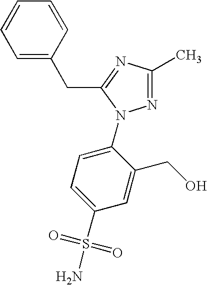

### Step1: 4-Hydrazino-3-(hydroxymethyl)benzenesulfonamide (k-1)

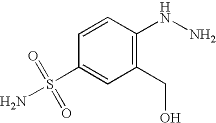

5-(Aminosulfonyl)-2-hydrazinobenzoic acid (j-1, 2.0 g, 8.65 mmol) was suspended in dry THF (100 mL) at 0° C. under argon, BH₃.THF (34.6 mL of 1M in THF, 34.6 mmol) was added dropwise. After the addition, the mixture was stirred at 0° C. for half hour, then the temperature was allowed to rise to room temperature. After stirred for 2 days, MeOH (80 mL) was added into the mixture at 0° C. carefully and stirred at room temperature for three hour, then three hours at 80° C. The solvent was removed. The white solid was dried under vacuum to give the title compound (1.69 g, yield 90%), which was used without further purification. ¹H NMR (300 MHz, DMSO-d₆) 4.21 (br. s., 2H) 4.40 (d, J=5.4 Hz, 2H) 5.29 (t, J=5.4 Hz, 1H) 6.84 (s, 1H) 6.94 (s, 2H) 7.14 (d, J=8.7 Hz, 1H) 7.55 (dd, J=8.7, 2.2 Hz, 1H) 7.60 (d, J=2.2 Hz, 1H).

### Step2: 4-(5-Benzyl-3-methyl-1H-1,2,4-triazol-1-yl)-3-(hydroxymethyl)-benzenesulfonamide (K-1)

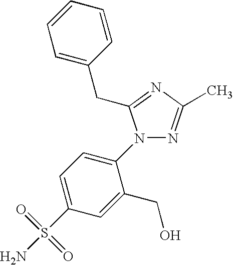

The title compound was made in similar fashion as described for Compound h-2. 4-Hydrazino-3-(hydroxymethyl)benzenesulfonamide j-1 (1.0 g, 4.6 mmol) and N-[(1E)-2-methoxy-1-methylethylidene]-2-phenylacetamide (1.04 g, 5.1 mmol, made according to the procedure for Compound h-1) gave the title compound as a white solid (1.05 g, yield 64%). NMR provided in Table. Elemental Analysis: Calcd for C₁₇H₁₈N₄O₃S.1.0 HCl.0.8 H₂O C, 49.89; H, 5.07; N, 13.69. Found: C, 49.83; H, 4.98; N, 13.46.

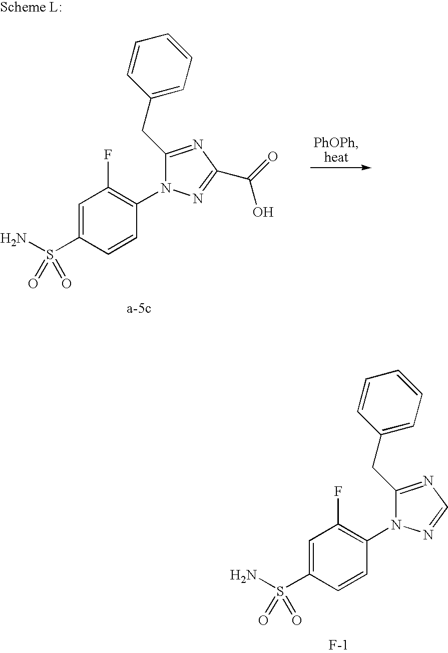

### Method L

### Example L-1

### 4-(5-benzyl-1H-1,2,4-triazol-1-yl)-3-fluorobenzenesulfonamide (L-1)

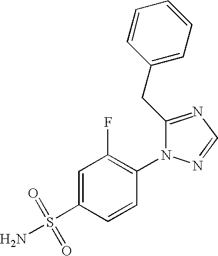

A solution of 1-[4-(aminosulfonyl)-2-fluorophenyl]-5-benzyl-1H-1,2,4-triazole-3-carboxylic acid (a-5c, 0.2 g, 0.53 mmol) in phenyl ether was stirred at 140° C. for 90 minutes. The reaction mixture was purified by reverse phase HPLC using acetonitrile in water (5 to 95%, with 0.1% trifluoroacetic acid). The resultant material was dissolved in methanol and 3N hydrochloride in methanol and the mixture stirred at room temperature for 5 minutes. The mixture was then concentrated to dryness, suspended in toluene and concentrated to dryness to give the hydrogen chloride salt of the title compound (0.021 g, 11%). NMR provided in Table. Elemental Analysis: Calcd for C₁₅H₁₃FN₄O₂S-1.1HCl-1.0H₂O C, 46.14; H, 4.16; N, 14.35. Found: C, 46.28; H, 3.81; N, 14.01.

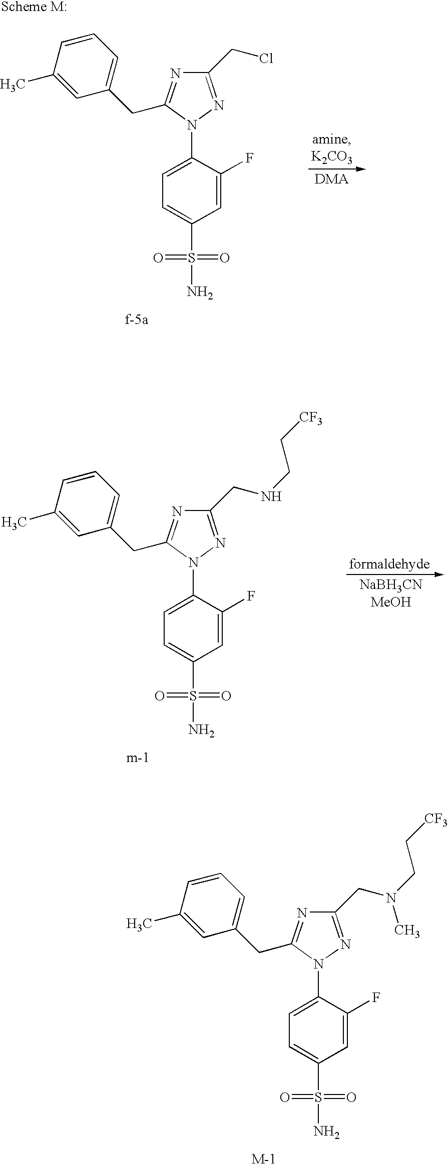

### Method M

### Example M-1

### 3-Fluoro-4-(5-(3-methylbenzyl)-3-{[methyl(3,3,3-trifluoropropyl)amino]methyl}-1H-1,2,4-triazol-1-yl)benzenesulfonamide (M-1)

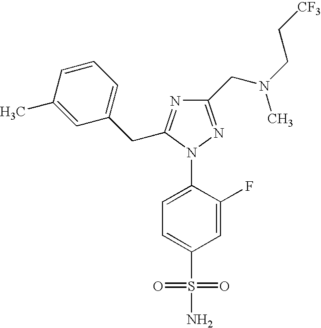

### Step 1: 3-Fluoro-4-(5-(3-methylbenzyl)-3-{[(3,3,3-trifluoropropyl)amino]methyl}-1H-1,2,4-triazol-1-yl)benzenesulfonamide (m-1)

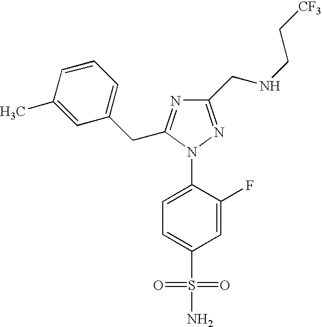

To a mixture of 4-[3-(chloromethyl)-5-(3-methylbenzyl)-1H-1,2,4-triazol-1-yl]-3-fluorobenzenesulfonamide f-5a (90 mg, 0.23 mmol, made in similar fashion as described in Method f for Compound f-5) in N,N-dimethylacetamide (3 mL) was added 3,3,3-trifluoropropan-1-amine hydrochloride (0.10 g, 0.68 mmol) followed by anhydrous potassium carbonate (160 mg, 1.1 mmol). The mixture was stirred at 50° C. for 16 hours. The mixture was cooled to room temperature, filtered, concentrated to dryness and the compound obtained as a brown oil (100 mg) was used for the next stage without additional purification. LCMS: (M+H)⁺: 472.20

### Step 2: 3-Fluoro-4-(5-(3-methylbenzyl)-3-{[methyl(3,3,3-trifluoropropyl)amino]methyl}-1H-1,2,4-triazol-1-yl)benzenesulfonamide (M-1)

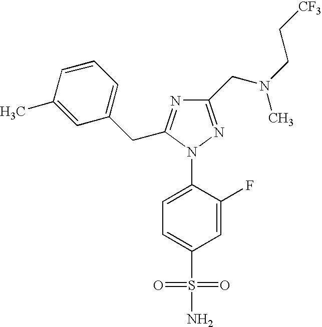

To a mixture of 3-fluoro-4-(5-(3-methylbenzyl)-3-{[(3,3,3-trifluoropropyl)amino]methyl}-1H-1,2,4-triazol-1-yl)benzenesulfonamide (m-1; 0.10 g, 0.23 mmol) in methanol (3 mL) was added 35% aqueous formaldehyde (54 μL, 0.68 mmol). After stifling for 20 minutes, sodium cyanoborohydride (43 mg, 0.68 mmol) was added. After stirring for 10 minutes, the mixture was made neutral with acetic acid and concentrated to dryness. Purification with preparative HPLC gave the title compound as a white solid (14 mg, 13%). NMR provided in Table. Elemental Analysis: Calcd for C₂₁H₂₃F₄N₅O₂S.1.1 HCl.1.1 H₂O C, 46.24; H, 4.86; N, 12.84. Found: C, 46.45; H, 4.81; N, 12.50.

Example M-1a was prepared from the appropriate starting material in a manner analogous to the methods of Example M-1.

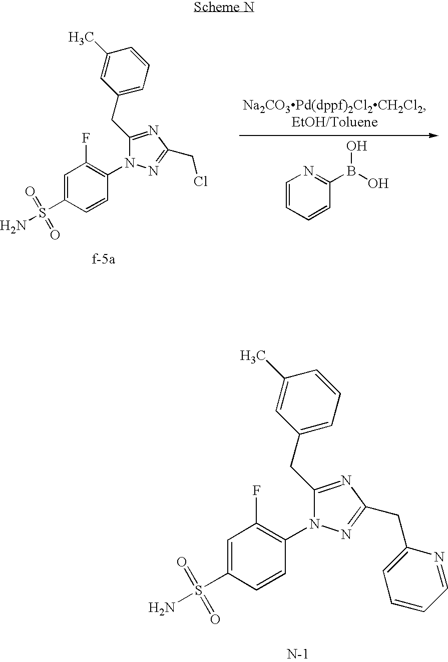

### Method N

### Example N-1

### 3-Fluoro-4-[5-(3-methylbenzyl)-3-(pyridin-2-ylmethyl)-1H-1,2,4-triazol-1-yl]benzenesulfonamide (N-1)

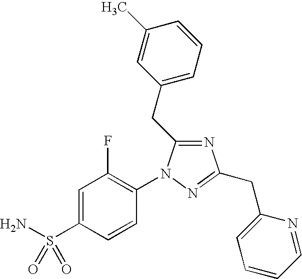

To a solution of 4-[3-(chloromethyl)-5-(3-methylbenzyl)-1H-1,2,4-triazol-1-yl]-3-fluorobenzenesulfonamide (f-5a, 0.30 g, 0.76 mmol) and Pd(II)(dPPf)₂Cl₂—CH₂Cl₂ (0.031 g, 0.040 mmol; Aldrich) in ethanol:toluene (3 mL, 1:2) was added pyridin-2-ylboronic acid (0.037 g, 0.3 mmol) and followed by 2.5M aqueous cesium carbonate (0.3 mL, 0.76 mmol). The mixture stirred at 75° C. for 48 h. The crude reaction mixture was purified by reverse phase HPLC using acetonitrile in water (5 to 95%, with 0.1% acetic acid) to give 26 mg (7%) of the acetate salt of the title compound. NMR provided in Table. Elemental Analysis: Calcd for C₂₂H₂₀FN₅O₂S-1.7AcOH-2.1H₂O C, 52.67; H, 5.43; N, 12.09. Found: C, 52.39; H, 4.88; N, 12.35.

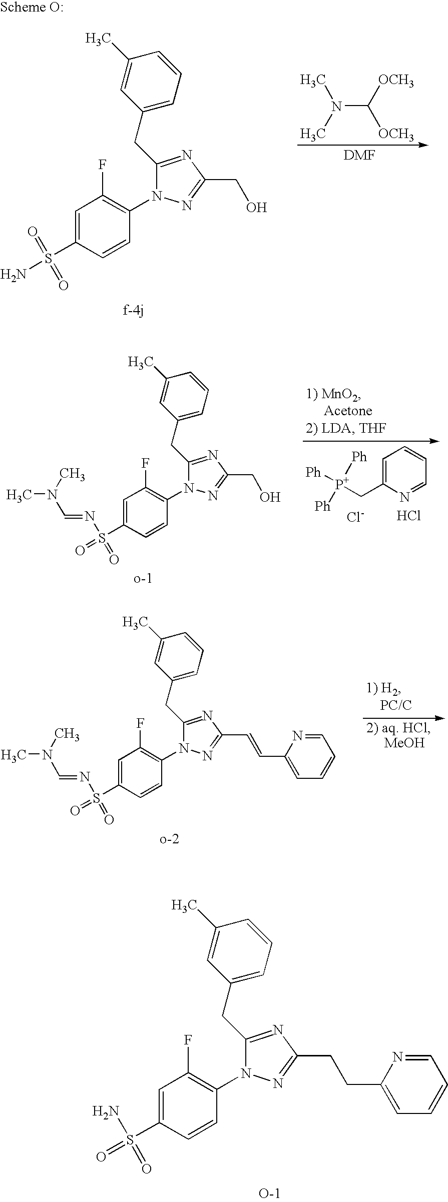

### Method O

### Example O-1

### 3-Fluoro-4-[5-(3-methylbenzyl)-3-(2-pyridin-2-ylethyl)-1H-1,2,4-triazol-1-yl]benzenesulfonamide (O-1)

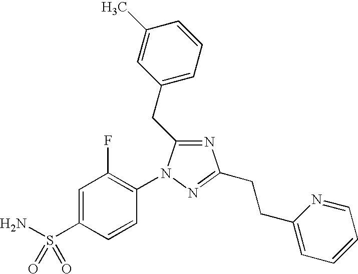

### Step 1: N-[(1E)-(Dimethylamino)methylene)-3-fluoro-4-[3-(hydroxymethyl)-5-(3-methylbenzyl)-1H-1,2,4-triazol-1-yl]benzenesulfonamide (o-1)

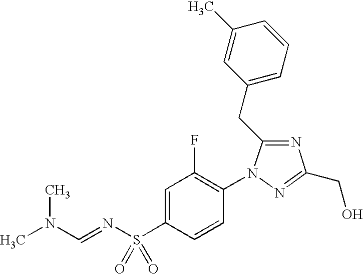

To a solution of f-4) (1.084 g, 2.05 mmol) in dimethylformamide (10 mL) was added dimethylformamide dimethyl acetal (0.329 g, 2.45 mmol). The mixture was stirred at room temperature for 18 hours, then diluted with ethyl acetate and water. The organic layer was further washed with water and concentrated in vacuo to give the title compound (0.68 g, 77%). HRMS Calcd for C₂₀H₂₂FN₅O₃S+[M+H+] 432.1500, found: 432.1499. ¹H NMR (300 MHz, DMSO-d₆) 2.20 (s, 3H) 2.98 (s, 3H) 3.20 (s, 3H) 4.05 (s, 2H) 4.48 (d, J=6.03 Hz, 2H) 5.39 (t, J=6.03 Hz, 1H) 6.80 (s, 1H) 6.85 (d, J=7.54 Hz, 1H) 7.00 (d, J=7.50 Hz, 1H) 7.12 (t, J=7.54 Hz, 1H) 7.70-7.87 (m, 3H) 8.30 (s, 1H).

### Step 2: N-[(1E)-(Dimethylamino)methylene]-3-fluoro-4-{5-(3-methylbenzyl)-3-[(E)-2-pyridin-2-ylvinyl]-1H-1,2,4-triazol-1-yl}benzenesulfonamide (o-2)

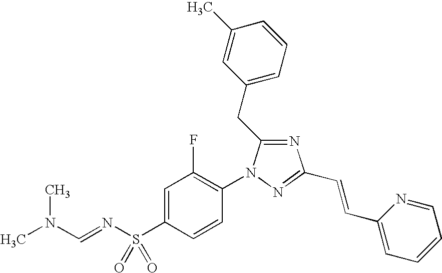

To a solution of N-[(1E)(dimethylamino)methylene]-3-fluoro-4-[3-(hydroxymethyl)-5-(3-methylbenzyl)-1H-1,2,4-triazol-1-yl]benzenesulfonamide (o-1, 0.477 g, 1.11 mmol) in acetone (5 mL) was added manganese dioxide (1.442 g, 16.58 mmol). The mixture was stirred at ambient temperature for 18 h. An additional 0.917 g (11.05 mmol) of manganese dioxide was added and the mixture was stirred at ambient temperature for 24 h. The reaction mixture was filtered through Celite, washing with ethyl acetate. The filtrate was concentrated in vacuo and the residue (0.212 g) used as such for the next step.

A suspension of triphenyl(2-pyridylmethyl)-phosphonium chloride hydrochloride (0.316 g, 0.74 mmol) in THF (2.5 mL) was cooled to −40° C. under nitrogen. To the suspension was added sodium bis(trimethylsilyl)amide (1.48 ml of 1M in THF; 1.48 mmol), and the reaction mixture was warmed to 5° C. over 40 min. To this mixture was added a solution of the crude N-[(1E)-(dimethylamino)methylene]-3-fluoro-4-[3-formyl-5-(3-methylbenzyl)-1H-1,2,4-triazol-1-yl]benzenesulfonamide (0.212 g, 0.490 mmol) in THF (1.5 mL) dropwise over 5 min at −70° C. The reaction mixture was warmed to ambient temperature and stirred overnight. The crude material was directly purified by reverse phase HPLC using acetonitrile in water (5 to 95%, with 0.1% acetic acid) to give 94 mg (38%) of the title compound. ¹H NMR (300 MHz, DMSO-d₆) 2.20 (s, 3H) 2.98 (s, 3H) 3.20 (s, 3H) 4.13 (s, 2H) 6.80 (s, 1H) 6.87 (d, J=7.54 Hz, 1H) 7.01 (d, J=7.54 Hz, 1H) 7.13 (t, J=7.54 Hz, 1H) 7.30-7.38 (m, 1H) 7.56 (s, 1H) 7.60 (s, 1H) 7.71 (d, J=7.91 Hz, 1H) 7.78-7.89 (m, 3H) 8.31 (s, 1H) 8.58-8.66 (m, 1H) HRMS Calcd for C₂₆H₂₅FN₆O₂S⁺[M+H⁺] 505.1817, found: 505.1815.

### Step 3: 3-Fluoro-4-[5-(3-methylbenzyl)-3-(2-pyridin-2-ylethyl)-1H-1,2,4-triazol-1-yl]benzenesulfonamide (O-1)

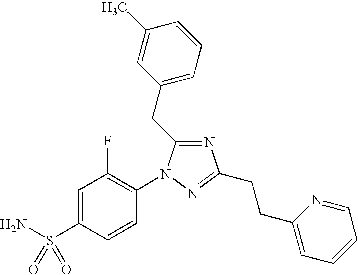

A suspension of N-[(1E)-(dimethylamino)methylene]-3-fluoro-4-{5-(3-methylbenzyl)-3-[(E)-2-pyridin-2-ylvinyl]-1H-1,2,4-triazol-1-yl}benzenesulfonamide (o-2, 0.075 g, 0.17 mmol) and 10% Pd/C (0.020 g) in methanol (1.25 mL) was stirred at ambient temperature for 4 h under hydrogen atmosphere. The mixture was diluted in dichloromethane and filtered through Celite. The filtrate was concentrated in vacuo and the residue (0.077 g) used as such for the next step.

A solution of residue (0.077 g, 0.15 mmol) in methanol (1 mL) and 5N aqueous HCl (2 mL) was stirred at 45° C. for 3 h, then at 60° C. for 18 h. The crude reaction mixture was purified by reverse phase HPLC using acetonitrile in water (5 to 95%, with 0.1% acetic acid) to give 7 mg (10%) of the acetate salt of the title compound. LRMS [M+H⁺] found: 452.1. ¹H NMR (300 MHz, MeOH-d₄) 2.24(s, 3H) 3.15-3.32 (m, 4H) 4.10 (s, 2H) 6.72-6.77 (m, 2H) 7.01 (d, J=7.50 Hz, 1H) 7.10 (t, J=7.91 Hz, 1H) 7.26-7.29 (m, 1H) 7.32 (d, J=7.72 Hz, 1H) 7.51 (t, J=7.80 Hz, 1H) 7.70-7.85 (m, 3H) 8.46-8.51 (m, 1H).

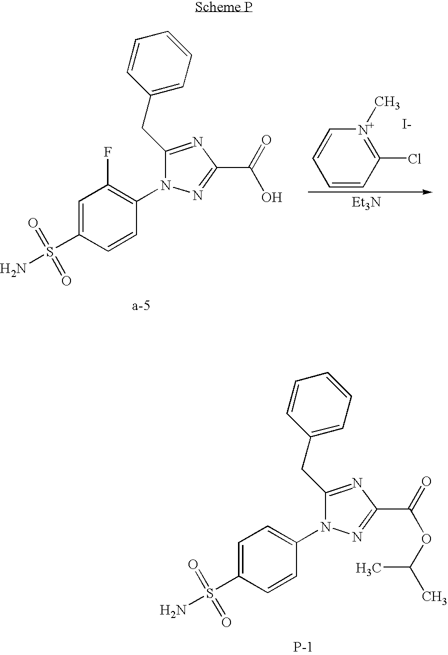

### Method P

### Example P-1

### Isopropyl 1-[4-(aminosulfonyl)phenyl]-5-benzyl-1H-1,2,4-triazole-3-carboxylate (P-1)

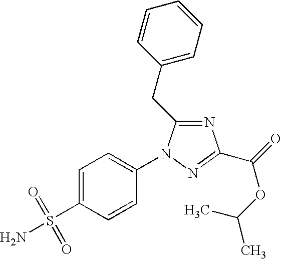

The title compound was made according to the procedure described in Kudo, et al, Chem. Pharm. Bull.; 1999, 47; 857-868. 1-[4-(Aminosulfonyl)phenyl]-5-benzyl-1H-1,2,4-triazole-3-carboxylic acid (a-5) and propan-2-ol gave the title compound in 56% yield as a white solid. NMR provided in Table. Elemental Analysis: Calcd for C₁₉H₂₀N₄O₄S-0.04Hexane: C, 57.21; H, 5.13; N, 13.87. Found: C, 57.21; H, 5.13; N, 13.87.

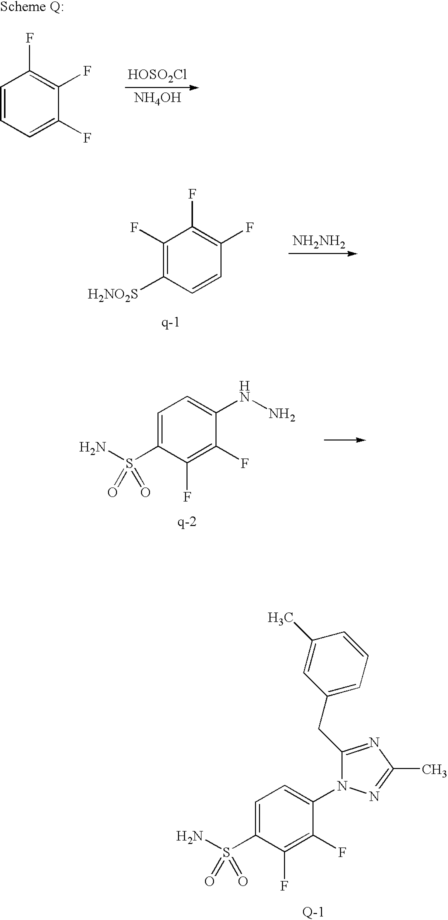

### Method Q

### Example Q-1

### 2,3-Difluoro-4-[3-methyl-5-(3-methylbenzyl)-1H-1,2,4-triazol-1-yl]benzenesulfonamide (Q-1)

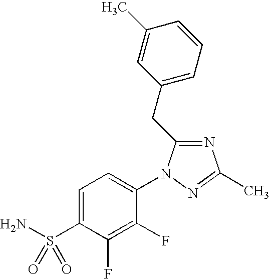

### Step 1: 2,3,4-Trifluorobenzenesulfonamide (q-1)

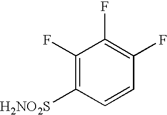

The title compound was made as a white solid (74% yield) in similar fashion as described for Compound kk-2. Elemental Analysis: Calcd for C₆H₄F₃NO₂S: C, 34.13; H, 1.91; N, 6.63. Found: C, 33.81; H, 2.00; N, 6.85. HRMS Calcd for C₆H₄F₃NO₂S Na⁺[M+Na⁺]: 233.9807, found: 233.9806. ¹H NMR (300 MHz, DMSO-d₆) 7.46-7.58 (m, 1H) 7.63-7.74 (m, 1H) 7.86 (br. s., 2H).

### Step 2: 2,3-Difluoro-4-hydrazinobenzenesulfonamide (q-2)

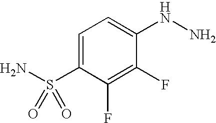

The title compound was made as a yellow solid (26% yield) in similar fashion as described for Compound j-1. HRMS Calcd for C₆H₈F₂N₃O₂S⁺[M+H⁺]: 224.0300, found: 224.0300. ¹H NMR (300 MHz, DMSO-d₆) d 4.14 (s., 2H) 8.35 (d, J=2.6 Hz, 1H) 6.69 (t, J=8.3 Hz, 1H) 7.17 (s., 2H) 7.32 (dd, J=8.9, 1.2 Hz, 1H).

The regiochemistry was confirmed with data most consistent with the shown structure. 2D COSY ¹H NMR, HMQR correlating ¹H and ¹³C assignments, and ¹³C NMR shifts matched calculated. (ACD Labs software CNMR predictor module, Version 8.0).

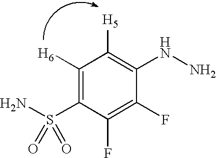

### Step 3: 2,3-Difluoro-4-[3-methyl-5-(3-methylbenzyl)-1H-1,2,4-triazol-1-yl]benzenesulfonamide (Q-1)

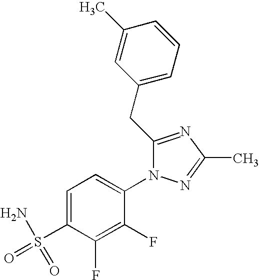

The title compound was made as a white solid (2% yield) in similar fashion as that described for Example f-3. MS: [M+H⁺]: 379.1. ¹H NMR (300 MHz, Methanol-d₄) d ppm 2.24 (s, 3H) 2.44 (s, 3H) 4.02 (d, J=16.50 Hz, 1H) 4.14 (d, J=16.50 Hz, 1H) 6.81-6.88 (m, 2H) 7.01 (d, J=7.70 Hz, 1H) 7.08 (t, J=7.72 Hz, 1H) 7.61-7.73 (m, 1H) 7.95-8.63 (m, 1H).

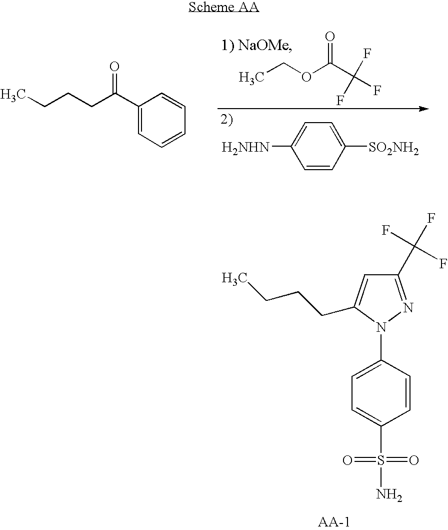

### Method AA

### Example AA-1

### 4-[5-(4-Butylphenyl)-3-(trifluoromethyl)-1H-pyrazol-1-yl]benzenesulfonamide (AA-1)

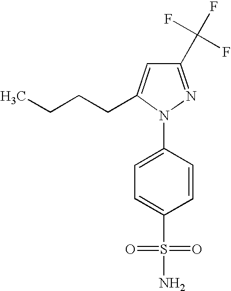

To a solution of sodium methoxide (0.48 mL of 25% in methanol, 2.1 mmol) in methanol (7 mL) was added 4-butylacetophenone (0276 g, 1.50 mmol). The mixture was stirred at ambient temperature for 5 minutes, and ethyl trifluoroacetate (0.358 g, 3.00 mmol) was added. The mixture was then stirred at 64° C. for 18 h. The solution was poured into 1N aq. HCl and extracted 3 times with dichloromethane. The combined organic layer was concentrated to dryness. The residue was dissolved in ethanol (5 mL) and 4-sulfonamidophenylhydrazine (290 mg, 1.5 mmol) was added. The mixture was stirred at 77° C. for 18 h then partially concentrated. The residue was purified by silica gel chromatography using 10-40% gradient of ethyl acetate in hexanes to provide the title compound (0.517 g, 81%). Regioselectivity assumed by analogy to Penning, T. D.; et al J. Med. Chem. 40, 1997, 1347-1365. ¹NMR provided in Table. Elemental Analysis: Calcd for C₂₀H₂₀F₃N₃O₂S: C, 56.73; H, 4.76; N, 9.92. Found: C, 56.82; H, 4.81; N, 9.82.

Examples AA-1a to AA-1g were prepared from the appropriate starting material in a manner analogous to the method of -Example AA-1.

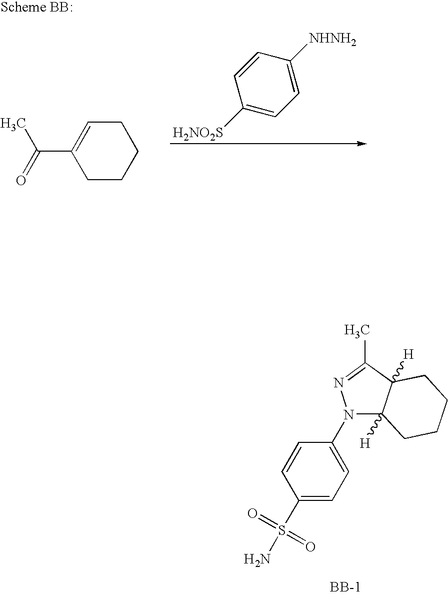

### Method BB

### Example BB-1

### 4-(3-Methyl-3a,4,5,6,7,7a-hexahydro-1H-indazol-1-yl)benzenesulfonamide (BB-1)

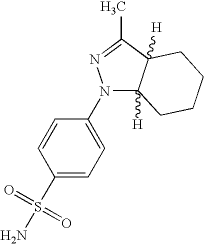

To a solution of 1-acetyl-cyclohexene (0.289 g, 2.2 mmol) in ethanol (10 mL) was added 4-sulfonamidophenylhydrazine (0.50 g, 2.24 mmol). The mixture was stirred at 75° C. for 18 h then partially concentrated. The residue was purified by silica gel chromatography using 25-60% ethyl acetate in hexanes to provide the title compound (0.293 g, 45%). ¹H NMR provided in Table. Elemental Analysis: Calcd for C₁₄H₁₉N₃O₂S C, 57.31; H, 6.53; N, 14.32. Found: C, 57.42; H, 6.48; N, 13.98.

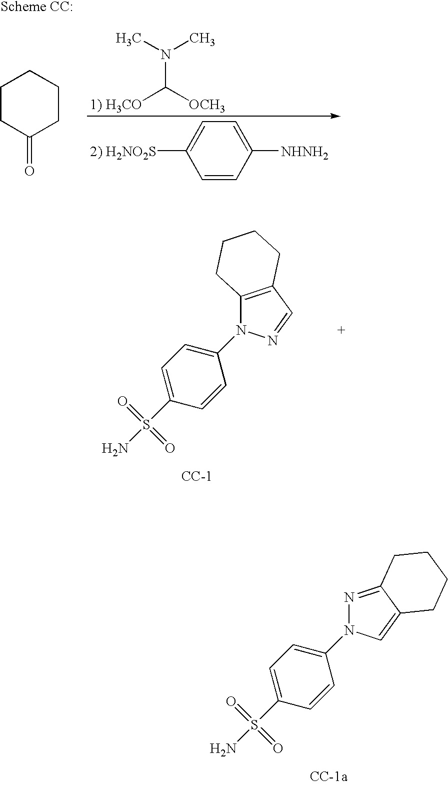

### Method CC

### Example CC-1 and CC-1a

### 4-(4,5,6,7-Tetrahydro-1H-indazol-1-yl)benzenesulfonamide (CC-1) and 4-(4,5,6,7-tetrahydro-2H-indazol-2-yl)benzenesulfonamide (cc-1a)

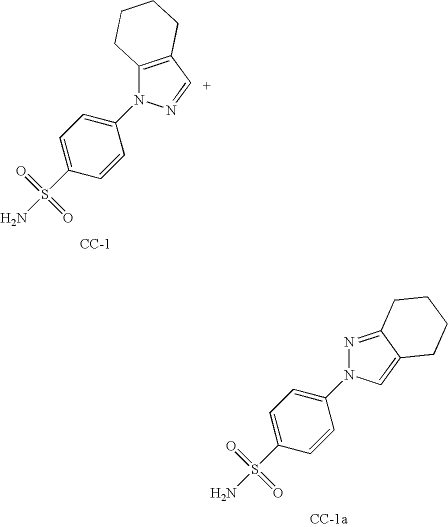

N,N-Dimethylformamide dimethyl acetal (1.33 g, 10 mmol) was added dropwise to neat cyclohexanone (1.033 g, 10 mmol) under nitrogen at ambient temperature. The mixture was stirred at 120° C. for 18 h. The mixture was cooled, and diluted with 20 mL of ethanol. 4-sulfonamidophenylhydrazine (1.675 g, 10 mmol) was added and the mixture stirred at 80° C. for 18 h. After cooling to ambient temperature, the reaction mixture was partially concentrated. The residue was purified by silica gel chromatography using 10-75% ethyl acetate in hexanes and further purified by reverse HPLC to provide the title compound CC-1 (1.814 g, 58%) and CC-1a (146 mg, 5.3%). ¹H NMR provided in Table. Elemental Analysis: CC-1, Calcd for C₁₃H₁₅N₃O₂S-0.2TFA-0.1H₂O C, 53.30; H, 5.14; N, 93.92. Found: C, 53.57; H, 5.39; N, 13.59. CC-1a, calcd for C₁₃H₁₅N₃O₂S-0.3TFA-0.7H₂O C, 50.39; H, 5.19; N, 12.96. Found: C, 50.61; H, 5.21; N, 12.54.

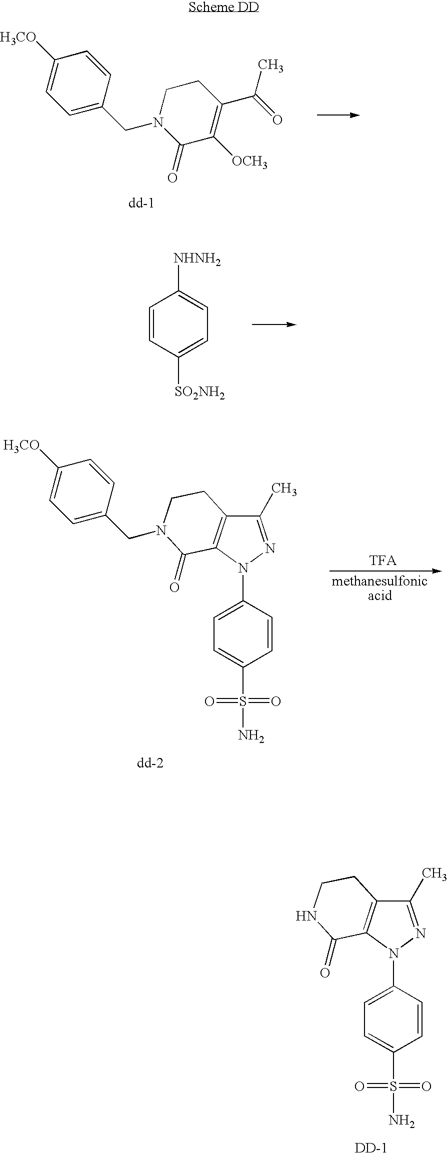

### Method DD

### Example DD-1

### 4-(3-Methyl-7-oxo-4,5,6,7-tetrahydro-1H-pyrazolo[3,4-c]pyridin-1-yl)benzene-sulfonamide (DD-1)

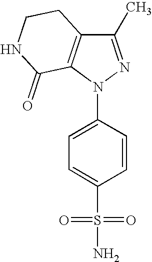

### Step 1: 4-Acetyl-3-methoxy-1-(4-methoxybenzyl)-5,6-dihydropyridin-2(1H)-one (dd-1)

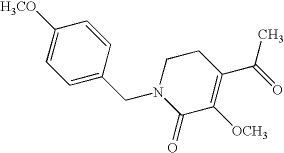

Made analogous to a route for 3-methoxy-1-(4-methoxybenzyl)-4-propionyl-5,6-dihydro-1H-pyridin-2-one from Urban, F. et al, Org. Proc. Res. & Dev. 2001, 5, 575-580.

### Step 2: 4-[6-(4-Methoxybenzyl)-3-methyl-7-oxo-4,5,6,7-tetrahydro-1H-pyrazolo[3,4-c]pyridin-1-yl]benzenesulfonamide (dd-2)

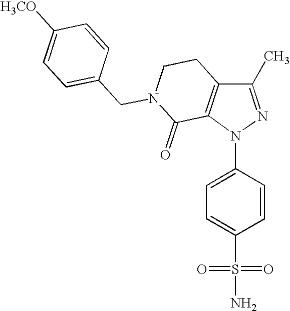

To 4-acetyl-3-methoxy-1-(4-methoxybenzyl)-5,6-dihydropyridin-2(1H)-one dd-1 (0.5 mmol) in ethanol (2 mL) was added 4-sulfonamidophenylhydrazine hydrochloride (0.122 g, 0.55 mmol). The mixture was stirred at 80° C. for 18 h. After cooling to ambient temperature, the reaction mixture was partially concentrated. The residue was purified by silica gel chromatography using a gradient of 50-100% ethyl acetate in hexanes to give 0.162 g of crude title compound dd-2. This material was used as such for the next step.

### Step 3: 4-(3-Methyl-7-oxo-4,5,6,7-tetrahydro-1H-pyrazolo[3,4-c]pyridin-1-yl)benzene-sulfonamide (DD-1)

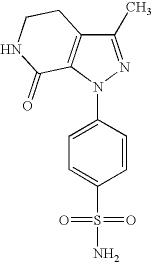

A solution of 4-[6-(4-methoxybenzyl)-3-methyl-7-oxo-4,5,6,7-tetrahydro-1H-pyrazolo[3,4-c]pyridin-1-yl]benzenesulfonamide (dd-2, 0.1 g, 0.2 mmol) in trifluoroacetic acid (1 mL) and methanesulfonic acid (0.03 mL) was stirred at 70° C. for 2 h. The mixture was then diluted in water and concentrated to dryness. The residue was purified by reverse phase HPLC using acetonitrile in water (5 to 95%, with 0.1% trifluoroacetic. acid). The resulting material was dissolved in methanol and 4N HCl in dioxane and the mixture stirred at ambient temperature for 5 minutes. The mixture was then concentrated to dryness to give the hydrochloride salt of the title compound (0.043 g, 62%). ¹H NMR provided in Table.

Elemental Analysis: Calcd for C₁₃H₁₄N₄O₃S-1.0HCl-3.0H₂O-0.4Dioxane C, 37.07; H, 5.16; N, 11.84. Found: C, 36.68; H, 4.94; N, 11.52.

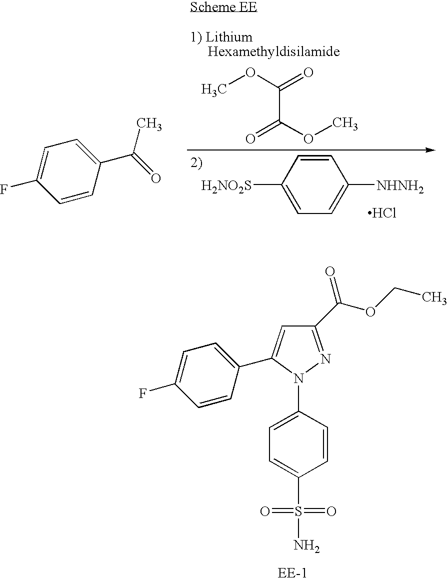

### Method EE

### Example EE-1

### Ethyl 1-[4-(aminosulfonyl)phenyl]-5-(4-fluorophenyl)-1H-pyrazole-3-carboxylate (EE-1)

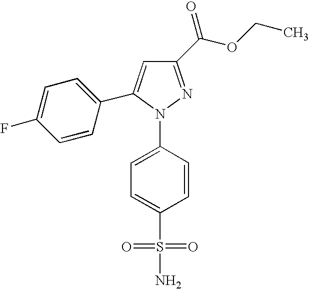

To a solution of lithium hexamethyldisilamide (4 mL of 1M in THF, 4 mmol) was slowly added 4-fluoro-acetophenone (0.242 mL, 4.00 mmol). The mixture was stirred at ambient temperature for 5 minutes, and dimethyl oxalate (0.354 g, 3.00 mmol) was added. The mixture was then stirred at ambient temperature for 18 h. The solution was poured into 1N aq. HCl and extracted 3 times with dichloromethane. The combined organic layer was concentrated to dryness. The residue was dissolved in ethanol (5 mL). 4-Sulfonamidophenylhydrazine hydrochloride (0.335 g, 1.50 mmol) was added. The mixture was stirred at 77° C. for 18 h then partially concentrated. The residue was purified by silica gel chromatography using 20-75% ethyl acetate in hexanes to provide the title-compound Q-1 (0.454 g, 78%). Regloselectilvity assumed by analogy to Penning, T. D.; et al J. Med. Chem. 40, 1997, 1347-1365. ¹H NMR provided in Table.

Elemental Analysis: Calcd for C₁₈H₁₆FN₃O₄S C, 55.52; H, 4.14; N, 10.79. Found: C, 55.23; H, 4.18; N, 10.68.

Examples EE-1a to EE-1g were prepared from the appropriate starting material in a manner analogous to the method of Example EE-1.

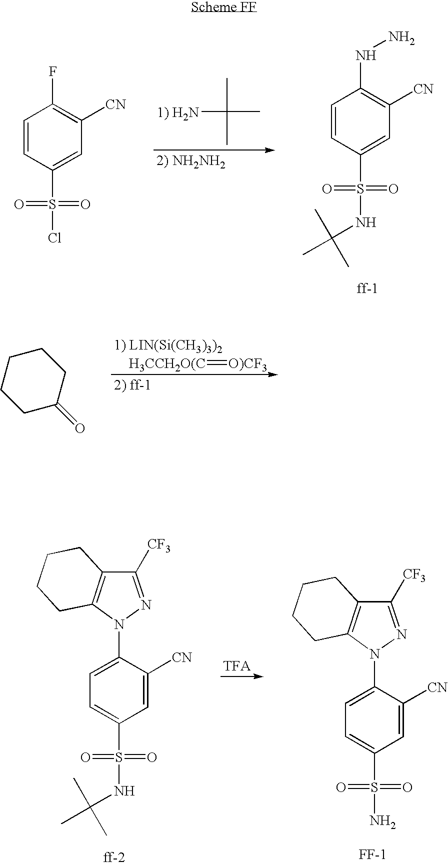

### Method FF

### Example FF-1

### 3-Cyano-4-[3-(trifluoromethyl)-4,5,6,7-tetrahydro-1H-indazol-1-yl]-benzenesulfonamide (FF-1)

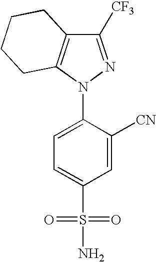

### Step 1: N-tert-Butyl-3-cyano-4-hydrazinylbenzenesulfonamide (ff-1)

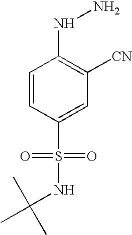

To a cooled to 5° C. solution of 4-fluoro-3-cyanobenzenesulfonyl chloride (5.00 g, 22.8 mmol) in dichloromethane was slowly added t-butylamlne (2.63 mL, 25.0 mmol). The mixture was stirred at 5° C. to ambient temperature for 18 h. The mixture was then concentrated and the residue dissolved in acetonitrile (100 mL). Hydrazine (1.2 mL, 50 mmol) was added and the mixture stirred at ambient temperature for 48 h. Additional hydrazine (0.6 mL, 25 mmol) was added and the mixture stirred at ambient temperature for 4 h. The mixture was concentrated, then dissolved in ethyl acetate and water. The aqueous layer was treated with 20 ml of 1N aqueous HCl and extracted twice with ethyl acetate. The combined organic layer was washed with diluted 0.2 N aqueous HCl and concentrated. The residue was purified by silica gel chromatography using 40-100% ethyl acetate in hexanes to provide the title compound (2.36 g, 39%). Elemental Analysis: Calcd for C₁₁H₁₆N₄O₂S C, 49.24; H, 6.01; N, 20.88. Found: C, 49.11; H, 5.94; N, 20.61. LRMS: 269.0 (M+H)⁺. ¹H NMR (400 MHz, DMSO-d₆) ppm 0.93-0.99 (m, 9H), 5.57 (br. s., 2H), 7.17 (s, 1H), 7.24 (d, J=8.84 Hz, 1H), 7.52 (dd, J=8.84, 1.77 Hz, 1H), 8.20 (d, J=1.26 Hz, 1H), 11.72 (s, 1H).

### Step 2: Ethyl 1-[4-(Aminosulfonyl)phenyl]-5-(4-fluorophenyl)-1H-pyrazole-3-carboxylate (ff-2)

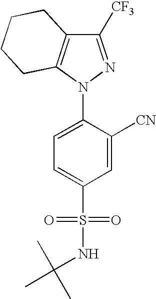

The procedure to prepare ethyl 1-[4-(aminosulfonyl)phenyl]-5-(4-fluorophenyl)-1H-pyrazole-3-carboxylate (EE-1) was used to prepare the title compound (ff-2) from ff-1 and crude 2-trifluoroacetyl-cyclohexanone prepared transiently in a manner similar to that of Example kk-1. Regioisomer assignment by analogy (spectral similarity) to the experiment that furnished three products, Example EE-1b, EE-1c, EE-1d. LCMS: (M+H)⁺: 427.1

### Step 3: 3-Cyano-4(3-(trifluoromethyl)-4,5,6,7-tetrahydro-1H-indazol-1-yl]benzenesulfonamide (FF-1)

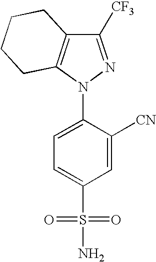

A solution of N-tert-butyl-3-cyano-4-(3-(trifluoromethyl)-4,5,6;7-tetrahydroindazol-1-yl)benzene-sulfonamide (0.1 g, 0.2 mmol) in TFA (2 mL) was heated at 50° C. for 2 h and then concentrated in vacuo. The residue was dissolved in methanol and 4N HCl in dioxane, stirred at ambient temperature for 10 min, and concentrated to give a solid (0.075 g, 92%). NMR provided in Table. Elemental Analysis: Calcd for C₁₅H₁₃F₃N₄O₂S-0.2HCl C, 47.71; H, 3.52; N, 14.84. Found: C, 47.96; H, 3.68; N, 14.44.

Example FF-1a was prepared from the appropriate starting material in a manner analogous to the method of Example FF-1.

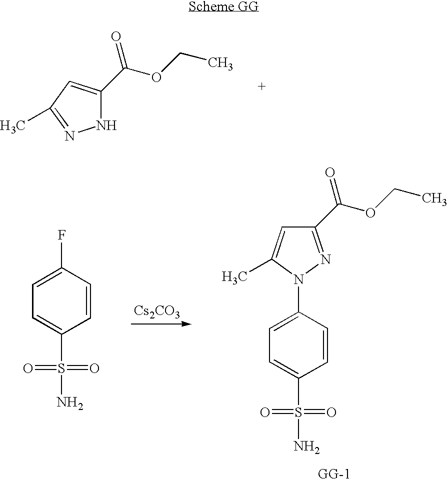

### Method GG

### Example GG-1

### Ethyl 1-[4-(Aminosulfonyl)phenyl]-5-methyl-1H-pyrazole-3-carboxylate GG-1

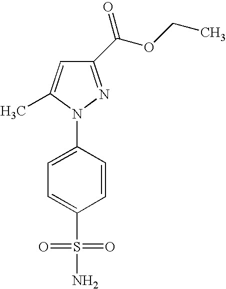

A solution of 4-fluorobenzene sulfonamide (4.00 g, 22.7 mmol), ethyl 3-methylpyrazole-5-carboxylate (3.50 g, 22.7 mmol) and cesium carbonate (14.8 g, 45.4 mmol) in dimethyl sulfoxide (25 mL) was heated at 110° C. for 3 days. The reaction mixture was diluted in water, saturated aqueous ammonium chloride and ethyl acetate. The aqueous layer was neutralized with 1N aqueous HCl and extracted with ethyl acetate. The combined organic layer was concentrated and the residue purified by silica gel chromatography using 25-75% ethyl acetate in hexanes to provide solid (0.79 g, 11%). NMR provided in Table.

The regiochemistry was confirmed with a ROESY ¹H NMR experiment that displayed a relationship between the C5′ methyl with the C3,5 aromatic hydrogens, as depicted on the structure below:

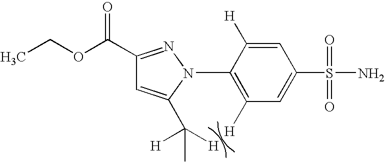

Elemental Analysis: Calcd for C₁₃H₁₅N₃O₄S C, 50.47; H, 4.89; N, 13.58. Found: C, 50.59; H, 4.89; N, 13.67.

Examples GG-1a to GG-1e were prepared from the appropriate starting material in a manner analogous to the method of Example GG-1.

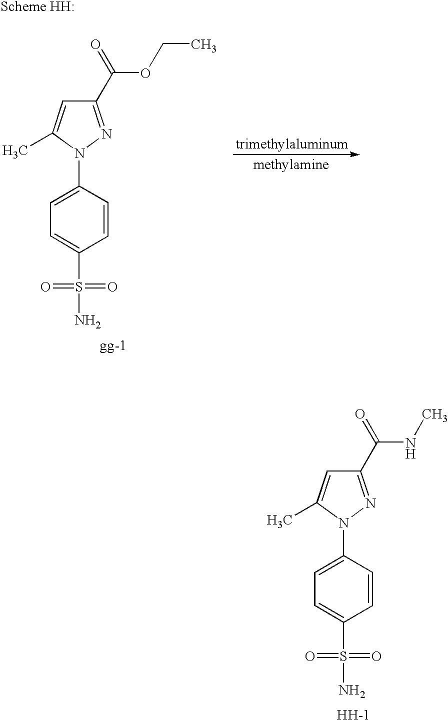

### Method HH

### Example HH-1

### 1-[4-(Aminosulfonyl)phenyl]-N,5-dimethyl-1H-pyrazole-3-carboxamide (HH-1)

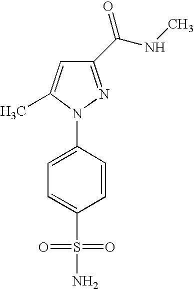

To a cold 0° C. solution of methylamine (x mL of 2M in tetrahydrofuran) in THF was slowly added trimethylaluminum (x mL of y M in ?). After 5 minutes at 0° C., the mixture was warmed to ambient temperature, then stirred for 1 h. Ethyl 1-[4-(aminosulfonyl)phenyl]-5-methyl-1H-pyrazole-3-carboxylate (gg-1; 0.099 g, 0.3 mmol) in tetrahydrofuran (1 mL) was slowly added. The mixture stirred at 65° C. for 2 h, allowed to cool, then dissolved in saturated aqueous ammonium chloride (1 mL) and water, and extracted with ethyl acetate. The combined organic layers were concentrated under reduced pressure and the residue purified by reverse phase HPLC using acetonitrile in water (5 to 95%, with 0.1% tritluoroacetic acid). The resulting material was dissolved in methanol/4N HCl in dioxane and stirred at ambient temperature for 5 minutes. The mixture was then concentrated to dryness to give the title compound (0.084 g, 46%). NMR provided in Table. Elemental Analysis: Calcd for C₁₂H₁₄N₄O₃S-0.3HCl C, 47.03; H, 4.73; N, 18.15. Found: C, 47.21; H, 4.72; N, 18.53.

Examples HH-1a to HH-1d were prepared from the appropriate starting material in a manner analogous to the method of Example HH-1.

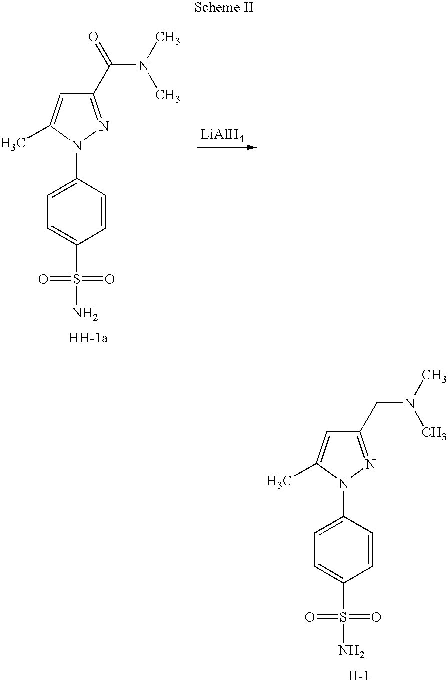

### Method II

### Example II-1

### 4-{3-[(Dimethylamino)methyl]-5-methyl-1H-pyrazol-1-yl}benzenesulfonamide (II-1)

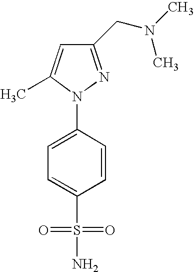

To a solution of 1-[4-(aminosulfonyl)phenyl]-N,N,5-trimethyl-1H-pyrazole-3-carboxamide (HH-1a;

0.088 g, 0.30 mmol, made according to the procedure from Example HH-1) in tetrahydrofuran was added lithium aluminum hydride (0.77 mL of 1M in tetrahydrofuran, 0.77 mmol). After 18 h at ambient temperature, the reaction mixture was slowly added to water and 2mL of 1M aqueous HCl. The aqueous layer was neutralized with 10N aqueous sodium hydroxide and extracted with ethyl acetate. The combined organic layers were concentrated and the residue was purified by reverse phase HPLC using acetonitrile in water (5 to 95%, with 0.1% trifluoroacetic acid). The resulting material was dissolved in methanol and 4N HCl in dioxane and the mixture stirred at ambient temperature for 5 minutes. The mixture was then concentrated to dryness to give the hydrochloride salt of the title compound (0.024 g, 22%). NMR provided in Table. Elemental Analysis: Calcd for C₁₃H₁₈N₄O₂S-2.0HCl-0.2Dioxane C, 43.14; H, 5.64; N, 14.30. Found: C, 43.06; H, 5.66; N, 14.56.

Examples II-1a to II-1c were prepared from the appropriate starting material in a manner analogous to the method of Example II-1.

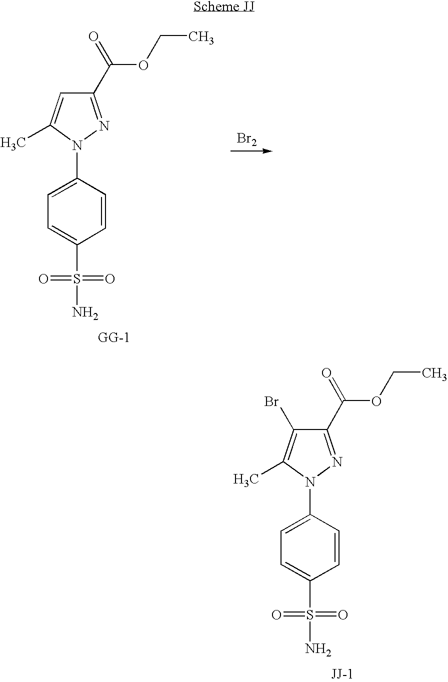

### Method JJ

### Example JJ-1

### Ethyl 1-[4-(Aminosulfonyl)phenyl]-4-bromo-5-methyl-1H-pyrazole-3-carboxylate JJ-1

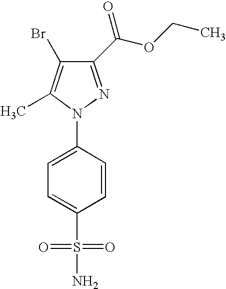

To a cooled to 0° C. solution of ethyl 1-[4-(aminosulfonyl)phenyl]-5-methyl-1H-pyrazole-3-carboxylate GG-1 (0.075 g, 0.2 mmol) in dicholoromethane (1 mL) was slowly added bromine (0.019 g, 0.36 mmol) in dicholoromethane (1 mL). The mixture was stirred at 0° C. for 1 h, then treated with 1 mL of saturated aqueous sodium bicarbonate. The organic layer was concentrated and the residue purified by silica gel chromatography using 0-10% methanol in dicholoromethane to provide the title compound (0.088 g, 94%). NMR provided in Table. Elemental Analysis: Calcd for C₁₃H₁₄BrN₃O₄S C, 40.22; H, 3.63; N, 10.82. Found: C, 40.19; H, 3.83; N, 10.51.

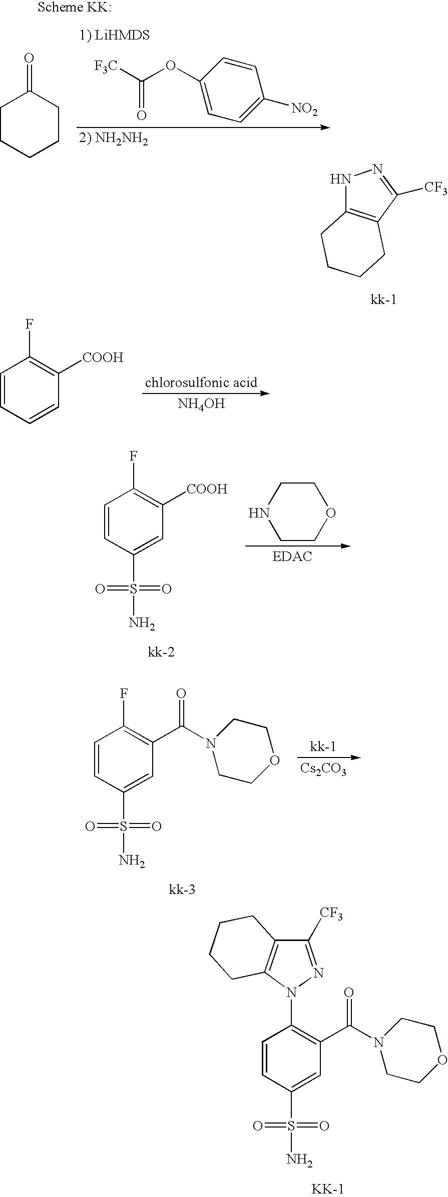

### Method KK

### Example KK-1

### 3-(Morpholin-4-ylcarbonyl)-4-4-[3-(trifluoromethyl)-4,5,6,7-tetrahydro-1H-indazol-1-yl]-benzene-sulfonamide (KK-1)

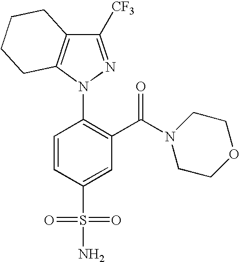

### Step 1: 3-(Trifluoromethyl)-4,5,6,7-tetrahydro-1H-indazole (kk-1)

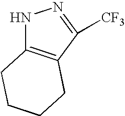

To lithium hexamethyldisilamide (75 mL of 1M in tetrahydrofuran, 75 mmol) at 0° C. was added dropwise a solution of cyclohexanone (3.37 g, 32.5 mmol) in tetrahydrofuran (25 mL). The reaction mixture was stirred at 0° C. for 15 min and 4-nitrophenyl 2,2,2-trifluoroacetate (11.46 mL, 48.75 mmol) was added. After 1 h at 0° C., hydrazine (4.76 g, 97.50 mmol) was added. After 18 h at 65° C., allowed to cool to ambient temperature, then slowly added concentrated 38% aqueous HCl (32.5 mL, 325 mmol). The mixture was warmed to 65° C. for 1 h, concentrated in vacuo, and purified via silica gel chromatography using a 5-25% gradient ethyl acetate in hexanes to provide the title compound (1.27 g, 21%). HRMS: 191.0798 (M+H)⁺. ¹H NMR (400 MHz, DMSO-d6) ppm 1.64-1.78 (m, 4 H) 2.48-2.53 (m, 2H) 2.61 (t, J=5.81 Hz, 2H) 13.09 (s, H)

### Step 2: 5-(Aminosulfonyl)-2-fluorobenzoic Acid (kk-2)

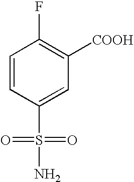

2-Fluorobenzoic acid (320 g, 2.28 mol) was added portionwise to chlorosulfonic add (750 mL) at ambient temperature. The resulting mixture was stirred at ambient temperature for 0.5 h and then heated at 130-140° C. overnight. The mixture was allowed to cool to ambient temperature and poured onto crushed ice slowly. The mixture was stirred for 0.5 h and the precipitate was filtered and washed with water thoroughly to afford a gray solid, which was added portionwise to 58% aq. ammonium hydroxide (700 ml) at 0° C. After stirring at ambient temperature overnight, the mixture was concentrated to a small volume under reduced pressure, and then carefully diluted with aq. H₂SO₄ at 0° C. and vigorous stirring until pH=5. The precipitate was filtered, washed with water and dried under vacuum at 50° C. to give 5-(aminosulfonyl)-2-fluorobenzoic acid (kk-2; 293 g, 58.7%), which was used without further purificattion. LRMS: 241.9 (M+Na)+. ¹H NMR (400 MHz, DMSO-d6) δ ppm 7.49-7.58 (m, 3H) 8.02-8.08 (m, 1H) 8.34 (dd, J=6.82, 2.53 Hz, 1H) 13.69 (br. s., 1H)

### Step 3: 4-Fluoro-3-(morpholine-4-carbonyl)benzenesulfonamide (kk-3)

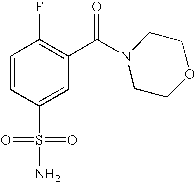

To a cooled to 0° C. solution of 5-(aminosulfonyl)-2-fluorobenzoic acid (0.504 g, 2.3 mmol) and 1-(3-dimethylaminopropyl)-3-ethylcarbodiimide hydrochloride (0.529 g, 2.8 mmol) in tetrahydrofuran (10 mL) was slowly added morpholine (0.401 mL, 4.6 mmol). After stirring at 0° C. for 5 minutes, the reaction was allowed to warm to ambient temperature and stirred overnight. DMF (1 mL) was then added and the mixture partially concentrated and stirred at ambient temperature for another 3 h. The mixture was then diluted with methylene chloride and water. The aqueous layer was further extracted with methylene chloride and the combined organic extracts were concentrated in vacuo. The crude 4-fluoro-3-(morpholine-4-carbonyl)benzenesulfonamide kk-3 was used as such without further purification.

### Step 4: 3-(Morpholin-4-ylcarbonyl)-4-[3-(trifluoromethyl)-4,5,6,7-tetrahydro-1H-indazol-1-yl]-benzenesulfonamide (KK-1)

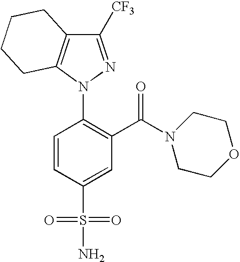

3-(Morpholin-4-ylcarbonyl)-4-[3-(trifluoromethyl)-4,5,6,7-tetrahydro-1H-indazol-1-yl]-benzenesulfonamide (KK-1) was prepared from crude 4-fluoro-3-(morpholine-4-carbonyl)benzenesulfonamide (kk-3) and 3-(trifluoromethyl)-4,5,6,7-tetrahydro-1H-indazole (kk-1) using the similar procedure as described in Example GG-1. NMR provided in Table. Elemental Analysis: Calcd for C₁₉H₂₁F₃N₄O₄S-2H₂O-0.5HCl-0.2Toluene C, 45.97; H, 5.13; N, 10.58. found: C, 45.84; H, 4.87; N, 10.17.

Examples KK-1a to KK-1U were prepared from the appropriate starting material in a manner analogous to the method of Example KK-1.

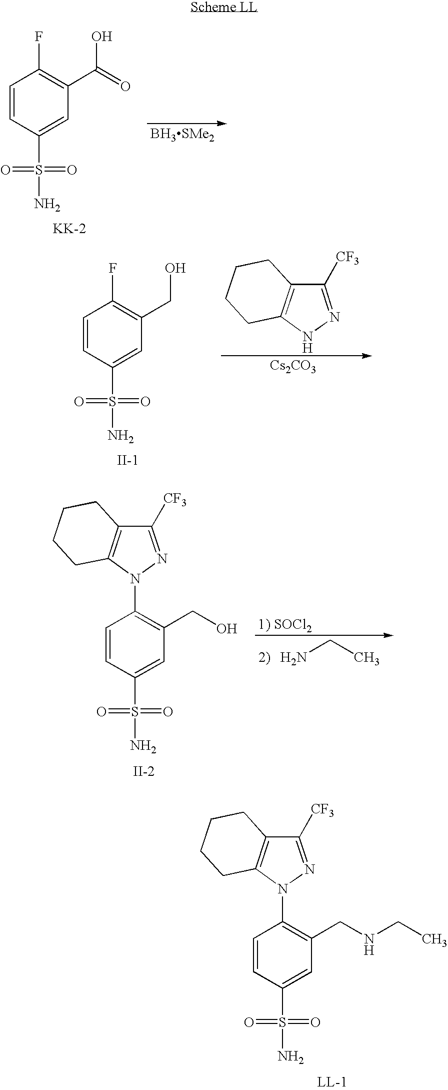

### Method LL

### Example LL-1

### 3-[(Ethylamino)methyl]-4-[3-(trifluoromethyl)-4,5,6,7-tetrahydro-1H-indazol-1-yl]-benzene-sulfonamide (LL-1)

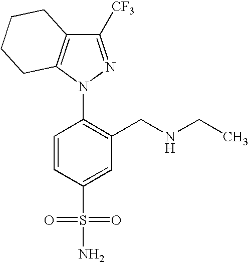

### Step 1: 4-Fluoro-3-(hydroxymethyl)benzenesulfonamide (II-1)

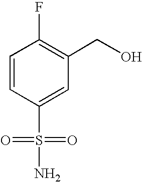

To a solution of 5-(aminosulfonyl)-2-fluorobenzoic acid (kk-2) in dry tetrahydrofuran (15 mL) was slowly added borane-dimethyl sulfide (4.56 mL of 2M in THF, 9.10 mmol), accompanied by effervescence. After 4 h of stirring at 27° C., the mixture was warmed to 65° C., then allowed to cool to ambient temperature, and additional borane-dimethyl sulfide was added. The mixture stirred at ambient temperature until the starting material was consumed. Methanol was added and the mixture concentrated under rotary evaporation to give crude 4-fluoro-3-(hydroxymethyl)benzenesulfonamide (II-1; 1.15 g, 100%), which was used without further purification. LCMS: (M−H)⁻: 203.9

### Step 2: 3-(Hydroxymethyl)-4-[3-(trifluoromethyl)-4,5,6,7-tetrahydro-1H-indazol-1-yl]benzenesulfonamide (II-2)

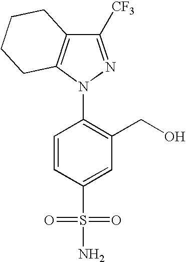

3-(Hydroxymethyl)-4-[3-(trifluoromethyl)-4,5,6,7-tetrahydro-1H-indazol-1-yl]benzenesulfonamide was prepared from crude 4-fluoro-3-(hydroxymethyl)benzenesulfonamide (II-1) and 3-(trifluoromethyl)-4,5,6,7-tetrahydro-1H-indazole (kk-1) using the procedure described in Example GG-1. Regiochemistry was assumed from similar spectral data for compounds from Examples FF-1, EE-1b, c, d and literature (see Bertenshaw, S. R., et al Bioorg. Med. Chem. Lett. 1996, 6, 2827-2830). NMR provided in Table. Elemental Analysis: Calcd for C₁₅H₁₆F₃N₃O₃S-0.05Toluene C, 48.52; H, 4.35; N, 11.06. Found: C, 48.62; H, 4.58; N, 10.73.

### Step 3: 3-[(Ethylamino)methyl]-4-[3-(trifluoromethyl)-4,5,6,7-tetrahydro-1H-indazol-1-yl]-benzenesulfonamide (LL-1)

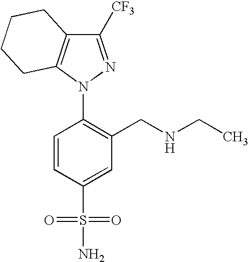

A mixture of 3-(hydroxymethyl)-4-[3-(trifluoromethyl)-4,5,6,7-tetrahydro-1H-indazol-1-yl]benzene-sulfonamide (II-2; 0.126 g, 0.336 mmol) in thionyl chloride (2 mL) stirred at 40° C. for 30 min, then concentrated to dryness. Ethylamine (5 mL of 2M in tetrahydrofuran, 10 mmol) was added. The mixture stirred at 40° C. for 2 h, concentrated, and purified by reverse phase HPLC using acetonitrile in water (5 to 95% gradient, with 0.1% trifluoroacetic acid). The resulting material was dissolved in methanol/4N HCl in dioxane, stirred at ambient temperature for 5 minutes, and concentrated to dryness to give the hydrochloride salt of the title compound LL-1 (0.012 g, 8%). NMR provided in table. HRMS calcd for C₁₇H₂₁F₃N₄O₂S⁺[M+H⁺] 403.1410, found: 463.1408.

Example II-2a was prepared from the appropriate starting material in a manner analogous to the method of Example II-2.

Example LL-1a was prepared from the appropriate starting material in a manner analogous to the method of Example LL-1.

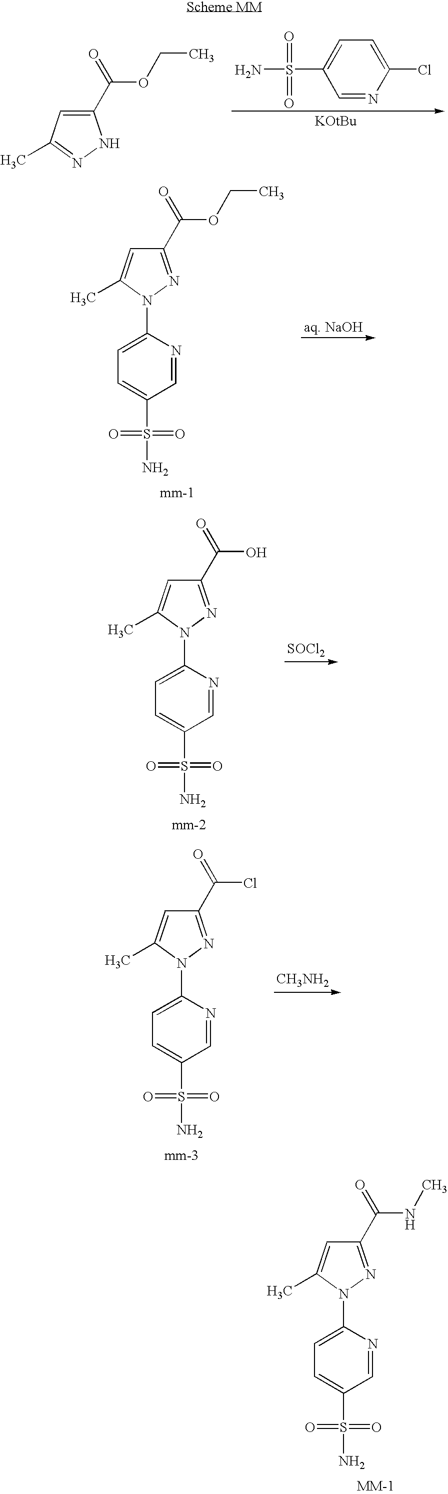

### Method MM

### Example MM-1

### 1-[5-(Aminosulfonyl)pyridin-2-yl]-N,5-dimethyl-1H-pyrazole-3-carboxamide (MM-1)

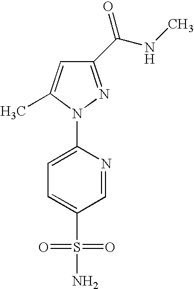

### Step 1: Ethyl 1-[5-(Aminosulfonyl)pyridin-2-yl]-5-methyl-1H-pyrazole-3-carboxylate (mm-1)

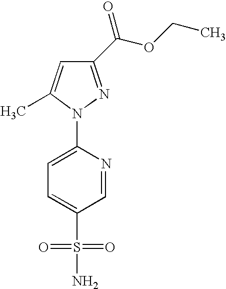

To a solution of ethyl 3-methylpyrazole-5-carboxylate (500 mg, 3.24 mmol) in 5 mL of anhydrous DMSO was added 6-chloropyridine-3-sulfonamide (623 mg, 3.24 mmol, Adams; J. Amer. Chem. Soc.; 1949; 71; 387-390), followed by the addition of potassium tert-butoxide (400 mg, 3.56 mmol). The mixture was heated at 100° C. in microwave for half hour. HPLC analysis showed one third of the starting material was consumed. The mixture was heated at 100° C. for one more hour and diluted with water (50 mL) with rapidly stirring. The resultant suspension was filtered. The white solid was washed with water and dried under vacuum to give the title compound mm-1 (0.75 g, yield 75%), which was used without further purification. NMR provided in Table.

The regiochemistry was confirmed with a 2D NOESY ¹H NMR experiment that displayed a relationship as depicted on the structure below:

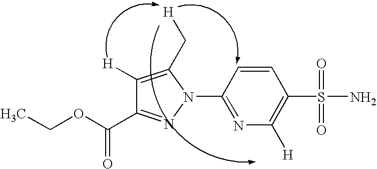

Elemental Analysis: Calcd for C₁₂H₁₄N₄O₄S: C, 46.44; H, 4.55; N, 18.05. Found: C, 46.48; H, 4.56; N, 17.96.

### Step 2: 1-[5-(Aminosulfonyl)pyridin-2-yl]-5-methyl-1H-pyrazole-3-carboxylic Acid (mm-2)

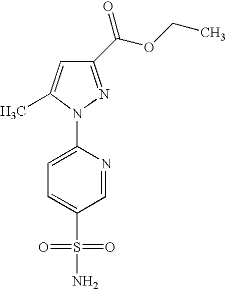

To a mixture of ethyl 1-[5-(aminosulfonyl)pyridin-2-yl]-5-methyl-1H-pyrazole-3-carboxylate (mm-1; 2.51 g, 8.08 mmol) in EtOH (20 mL) and water (10 mL) was added 4N NaOH (6.1 mL, 24.3 mmol). After one hour at 50° C., the mixture was cooled to 0° C. and acidified with 6 N HCl to pH 4. The white solid was washed with water and dried under vacuum to give the title compound mm-2 (2.1 g, yield 92%), which was typically used without further purification. NMR provided in Table. Elemental Analysis: Calcd for C₁₀H₁₀N₄O₄S: C, 42.55; H, 3.57; N, 19.85. Found: C, 42.64; H, 3.67; N, 19.68.

### Step 3: 1-[5-(Aminosulfonyl)pyridin-2-yl]-5-methyl-1H-pyrazole-3-carbonyl Chloride (mm-3)

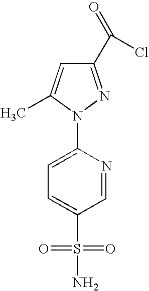

To 1-[5-(aminosulfonyl)pyridin-2-yl]-5-methyl-1H-pyrazole-3-carboxylic acid (mm-2, 500 mg, 1.77 mmol) under argon was added thionyl chloride (10 mL) at 0° C., followed by the addition of 2 drops of anhydrous DMF. The suspension was heated at 70° C. for 5 hours. The resultant solution was evaporated to dryness and azeotroped with toluene to give the title compound mm-3 (533 mg, 100% yield) as a white solid, which was used without further purification.

### Step 4: 1-[5-(Aminosulfonyl)pyridin-2-yl]-N,5-dimethyl-1H-pyrazole-3-carboxamide (MM-1)

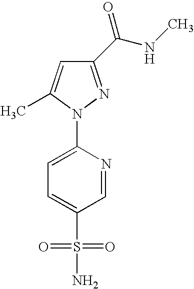

To a solution of 1-[5-(aminosulfonyl)pyridin-2-yl]-5-methyl-1H-pyrazole-3-carbonyl chloride (mm-3, 177 mg, 0.590 mmol) in THF (2 mL) at 0° C. was added methylamine (1.0 mL of 2M in THF). The mixture was stirred at ambient temperature for 3 hours. The solvent was evaporated. The residue was purified with preparative HPLC using acetonitrile in water to provide the title compound MM-1 (123 mg, 71% yield) as a white fluffy powder. NMR provided in Table. Elemental Analysis: Calcd for C₁₁H₁₃N₅O₃S.0.2 H₂O.0.1 TFA: C, 43.45; H, 4.38; N, 22.57. Found: C, 43.42; H, 4.69; N, 22.33.

Examples MM-1a to MM-1d were prepared from the appropriate starting material in a manner analogous to the method of Example MM-1.

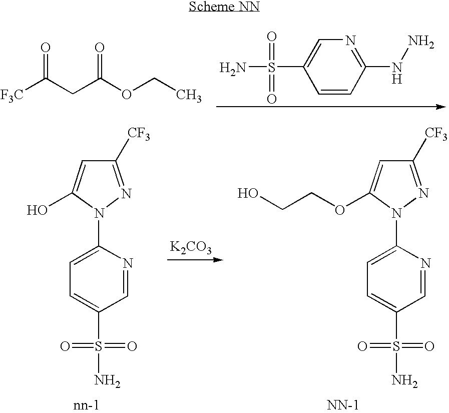

### Method NN

### Example NN-1

### 6-[5-(2-Hydroxyethoxy)-3-(trifluoromethyl)-1H-pyrazol-1-yl]pyridine-3-sulfonamide (NN-1)

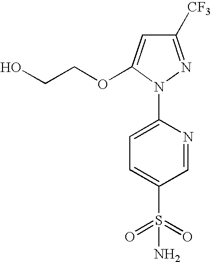

### Step 1: 6-[5-Hydroxy-3-(trifluoromethyl)-1H-pyrazol-1-yl]pyridine-3-sulfonamide (nn-1)

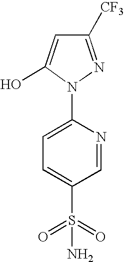

To ethyl 4,4,4-trifluoroacetoacetate (389 μL, 2.66 mmol) in 98% HOAc (1.2 mL) was added 6-hydrazinopyridine-3-sulfonamide (500 mg, 2.66 mmol). The mixture was heated at 110° C. for 3 hours. The suspension became clear solution and allowed to cool. The mixture was diluted with water and filtered. The white solid was washed with water and dried under vacuum to give the title compound nn-1 (245 mg, yield 55%), which was used without further purification. ¹H NMR (300 MHz, DMSO-d₆) 6.04 (s, 1H) 7.74 (s, 2H) 8.02 (d, J=8.7 Hz, 1H) 8.40 (dd, J=8.7, 2.5 Hz, 1H) 8.92 (d, J=2.5 Hz, 1H). LCMS: 305.90 [M−H⁺]

### Step 2: 6-[5-(2-Hydroxyethoxy)-3-(trifluoromethyl)-1H-pyrazol-1-yl]pyridine-3-sulfonamide (NN-1)

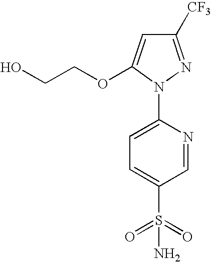

To the mixture of 6-[5-hydroxy-3-(trifluoromethyl)-1H-pyrazol-1-yl]pyridine-3-sulfonamide nn-1 (200 mg, 0.65 mmol) and 2-bromoethanol (138 μL, 1.95 mmol) in anhydrous DMF (2 mL) was added potassium carbonate (270 mg, 1.95 mmol). The mixture was heated at 150° C. in a microwave apparatus for 10 minutes. The mixture was extracted with ethylester, the concentrated organic layer was purified with preparative HPLC using acetonitrile in water to give the title compound NN-1 (104 mg, 45% yield) as a white solid. NMR provided in Table. Elemental Analysis: Calcd for C₁₁H₁₁F₃N₄O₄S: C, 37.50; H, 3.15; N, 15.90. Found: C, 37.33; H, 3.05; N, 15.71

Examples NN-1a to NN-1c were prepared from the appropriate starting material in a manner analogous to the method of Example NN-1.

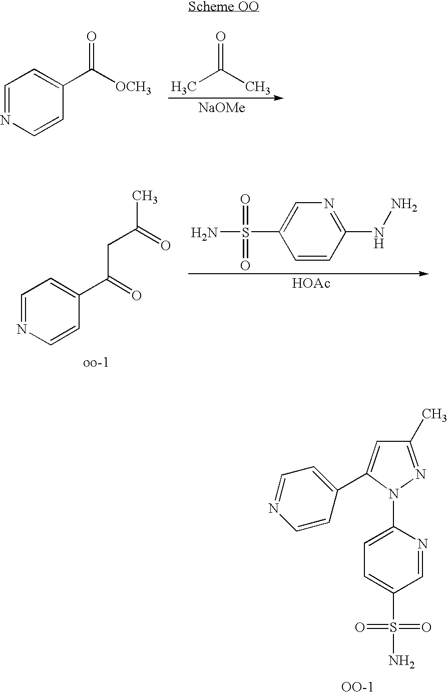

### Method OO

### Example OO-1

### 6-(3-Methyl-5-pyridin-4-yl-1H-pyrazol-1-yl)pyridine-3-sulfonamide (OO-1)

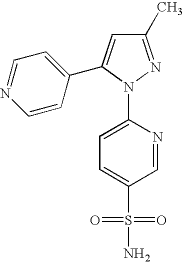

### Step1: 1-Pyridin-4-ylbutane-1,3-dione (oo-1)

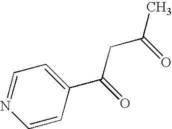

To a 500 mL flask with NaOMe (4.75 g) and anhydrous ether (130 mL) was sequentially added methyl isonicotinate (10.0 mL, 84.7 mmol), and a solution of acetone (12.4 mmol, 189 mmol) in ether (45 mL). The suspension was stirred at reflux for 6 hours, cooled, and filtered. The isolated solid was washed with ether and dissolved in water (40 mL). Glacial acetic acid (5.2 mL) was added and the mixture was extracted with chloroform (40 mL×2). The organic extracts were dried over anhydrous Na₂SO₄ and concentrated to give the title compound oo-1 (8.6 g, 62% yield) as a brown solid, which was used without further purification. LCMS: 164.10 [M+H⁺]

### Step 2: 6-(3-Methyl-5-pyridin-4-yl-1H-pyrazol-1-yl)pyridine-3-sulfonamide (OO-1)

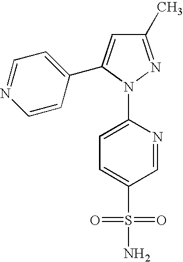

To 1-pyridin-4-ylbutane-1,3-dione 21-1 (245 mg, 1.50 mmol) in 1.5 mL of acetic acid was added 6-hydrazinopyridine-3-sulfonamide (282 mg, 1.5 mmol). The mixture was heated at 110° C. for one hour and cooled, then basified to pH 7 with Sat NaHCO₃. The resultant solid was filtered, washed with water and hexane/ether (1:1), then dried under vacuum to give the title compound U-1 (349 mg, yield 74%) as a light brown solid. Regiochemistry assumed from similar spectral data to compounds for literature, see Penning, T. D.; et al J. Med. Chem., 1997, 40, 1347-1355. NMR provided in Table. NMR provided in Table. Elemental Analysis: Calcd for C₁₄H₁₃N₅O₂S: C, 53.32; H, 4.16; N, 22.21. Found: C, 53.55; H, 4.19; N, 21.93.

Examples OO-1a to OO-1e were prepared from the appropriate starting material in a manner analogous to the method of Example OO-1.

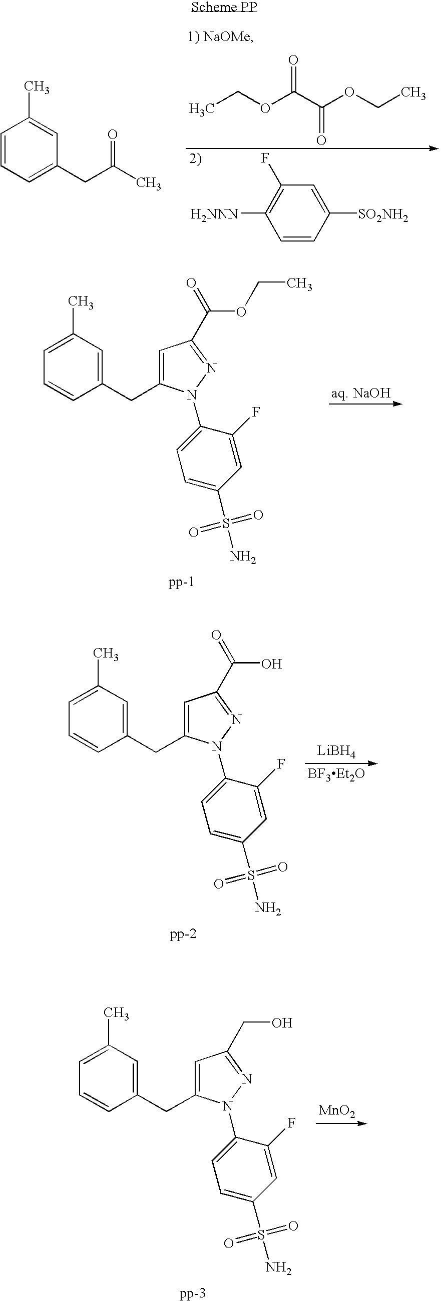

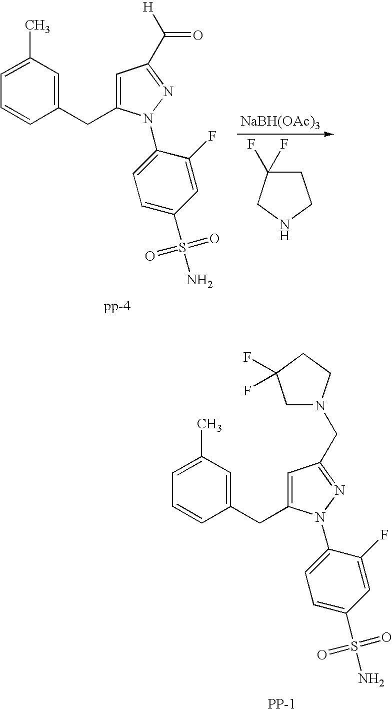

### Method PP

### Example PP-1

### 4-[3-[(3,3-difluoropyrrolidin-1-yl)methyl]-5-(3-methylbenzyl)-1H-pyrazol-1-yl]-3-fluorobenzenesulfonamide (PP-1)

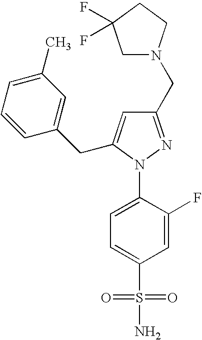

### Step 1: Ethyl 1-[4-(Aminosulfonyl)-2-fluorophenyl]-5-(3-methylbenzyl)-1H-pyrazole-3-carboxylate (pp-1)

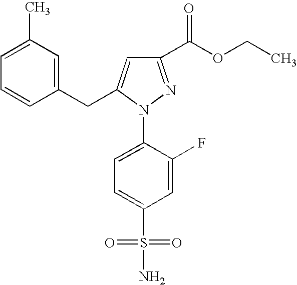

1-(3-Methylphenyl)acetone (32.4 g, 0.219 mol, Gardrat, C.; Bull. Soc. Chim. Belg.; 1984, 93; 897-902) was dissolved in THF (400 mL). Sodium methoxide (17.7 g, 0.328 mol) was added at −5-0° C. under stirring. After 5 min, a solution of diethyl oxalate (32 g, 0.219 mol) in ether (10 mL) was added dropwise. The mixture was heated to 60° C. for 30 min, stirred at this temperature for 3.5 h, and cooled to 5° C. Acetic acid (93 mL, 1.55 mol), absolute ethanol (400 mL), and 3-fluoro-4-hydrazinobenzenesulfonamide (38.2 g, 0.186 mol, Pal, M.; J. Med. Chem. 2003; 46; 3975-3984) were added. The mixture was slowly (for 2 h) heated to 60° C., stirred at this temperature overnight, cooled, and evaporated. The residue was stirred with a saturated solution of NaHCO₃ (500 mL), and the product was extracted with dichloromethane/methanol mixture, 9:1 (3×500 mL). The combined extracts were dried with sodium sulfate and evaporated. The residue was purified by chromatography (silica gel, ether/chloroform, 5:95) and used without further purification. Yield: 21.3 g (27.5%). LCMS: 418 [M+H⁺]. ¹H NMR (400 MHz, DMSO-d₆) d 1.27 (t, J=7.09 Hz, 3H), 2.18 (s, 3H), 3.91 (s, 2H), 4.28 (q, J=7.25 Hz, 2H), 6.70 (s, 1H), 6.74-6.79 (m, 2H), 6.97 (s, 1H), 7.09 (t, J=7.58 Hz, 1H), 7.68 (s, 2H), 7.72-7.83 (m, 3H).

### Step 2: 1-[4-(Aminosulfonyl)-2-fluorophenyl]-5-(3-methylbenzyl)-1H-pyrazole-3-carboxylic Acid (pp-2)

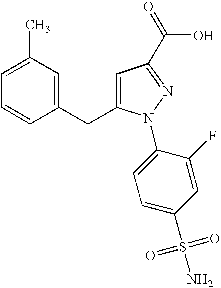

Ethyl 1-[4-(Aminosulfonyl)-2-fluorophenyl]-5-(3-methylbenzyl)-1H-pyrazole-3-carboxylate (pp-1, 21.3 g, 510 mmol) was dissolved in methanol (450 mL). A solution of NaOH (7.00 g, 175 mmol) in water (50 mL) was added dropwise and stirred at ambient temperature for 3 h. Solvent was evaporated. The residue was diluted with water (150 mL), and the solution was subjected to extraction with dichloromethane (2×100 mL). The aqueous layer was acidified to pH 4-2.5, and the product was extracted with chloroform/THF mixture, 4:1 (4×120 mL). The combined extracts were dried with Na₂SO₄ and evaporated to dryness to yield: 20.5 g (100%). LCMS: 390 [M+H⁺]. ¹H NMR (400 MHz, DMSO-d₆) d 2.19 (s, 3H), 3.85 (s, 2H), 6.47 (s, 1H), 6.72-6.80 (m, 2H), 6.97 (d, J=7.58 Hz, 1H), 7.09 (t, J=7.58 Hz, 1H), 7.63-7.83 (m, 3H).

### Step 3: 3-Fluoro-4-[3-(hydroxymethyl)-5-(3-methylbenzyl)-1H-pyrazol-1-yl]benzenesulfonamide (pp-3)

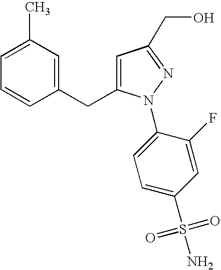

LiBH₄ (3.9 g, 0.178 mol) was suspended in THF (250 mL). BF₃.Et₂O (7.6 mL, 0.06 mol) was added dropwise under stirring and cooling with Ice. The cooling bath was removed. The mixture was stirred at ambient temperature for 40 min and cooled again. A solution of 1-[4-(aminosulfonyl)-2-fluorophenyl]-5-(3-methylbenzyl)-1H-pyrazole-3-carboxylic acid (pp-2) in THF (150 mL) was carefully added to control gas evolution. The reaction mixture was slowly heated to 55° C., stirred at this temperature for 3.5 h, and cooled. A mixture of methanol (300 mL) and 4 M HCl in dioxane (90 mL, 0.36 mol) was added dropwise. When vigorous gas evolution ceased, the mixture was slowly heated to 55° C., stirred at this temperature for 3 h, cooled, and evaporated. The residue was coevaporated with methanol (400 mL) and diluted with water (50 mL). A saturated solution of NaNCO₃ (100 mL) was added. The formed precipitate was separated by filtration, dried, and recrystallized from THF/benzene/hexane mixture, 1:1:1, which was used without further purification. Yield: 13.5 g (70.6%). LCMS: 376 [M+H⁺]. ¹H NMR (400 MHz, DMSO-d₆) δ 2.01-2.26 (m, 2H), 3.09-3.38 (m, 1H), 3.85 (s, 2H), 4.40 (t, J=6.11 Hz, 2H), 5.10 (t, J=5.87 Hz, 1H), 6.06-6.29 (m, 2H), 6.69-6.84 (m, 2H), 6.97 (d, J=7.09 Hz, 1H), 7.10 (t, J=7.83 Hz, 1H), 7.55-7.70 (m, 2H), 7.69-7.84(m, 2H).

### Step 4: 3-Fluoro-4-[3-formyl-5-(3-methylbenzyl)-1H-pyrazol-1-yl]benzenesulfonamide (pp-4)

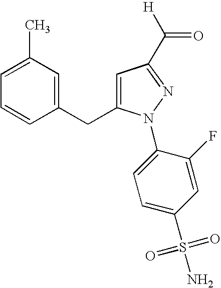

Activated MnO₂ (70 g, 0.70 mol, 88%) was suspended in dichloromethane (300 mL). A cooled solution of 3-fluoro-4-[3-(hydroxymethyl)-5-(3-methylbenzyl)-1H-pyrazol-1-yl]benzene-sulfonamide (pp-3, 13.5 g, 36 mmol) in a mixture of dichloromethane (300 mL), THF (300 mL), and acetone (200 mL) was added in one portion under argon with cooling ice bath. The reaction mixture was stirred at 0° C. for 3.5 h and filtered through Celite. The separated solid was washed with dichloromethane, and the filtrate was evaporated to provide a solid, which was used without further purification. Yield: 11 g (82%). LCMS: 374 [M+H⁺]. ¹H NMR (400 MHz, DMSO-d₆) d ppm 2.11-2.17 (s, 3H), 3.93 (s, 2H), 8.71-6.79 (m, 2H), 6.92-7.00 (m,1H), 7.04-7.11 (m, 1H), 7.69 (bs, 2H), 7.74-7.88-(m, 2H), 9.82-9.88 (s, 1H).

### Step 5: 4-[3-[(3,3-Difluoropyrrolidin-1-yl)methyl]-5-(3-methylbenzyl)-1H-pyrazol-1-yl]-3-fluorobenzenesulfonamide Hydrochloride (PP-1)

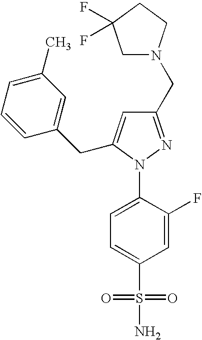

To a solution of the aldehyde pp-3 (100 mg, 0.3 mmol) in anhydrous DMA (2 mL) was added sequentially a solution of 3,3-difluoropyrrolidine (129 mg, 0.900 mmol) in 2 mL of DMA and triethylamine (86 μL, 0.900 mmol). To this solution a dispersion of NaBH(OAc)₃ (191 mg, 0.9 mmol) in MeCN (2 mL) was added. The reaction mixture was stirred at ambient temperature for 8 h. After quenching the excess hydride with water, the volatiles were removed and the residue fractioned between EtOAc and aqueous saturated NaHCO₃. The organic layer was dried over Na₂SO₄ and concentrated. The product was purified by loading into a Mega BE-SCX solid phase extraction column (Varian™). The fractions containing the product were concentrated to dryness and the residue was dissolved in 0.5 N HCl in Ethanol. After removing the volatiles the residue was diluted with water/ MeCN and freeze-dried to give the product as a white solid (89%). NMR provided in Table. LCMS: 465 [M+H⁺].

Examples pp-12a to pp-2b were prepared from the appropriate starting material in a manner analogous to the method of Example pp-2.

Examples PP-1a to PP-1b were prepared from the appropriate starting material in a manner analogous to the method of Example PP-1.

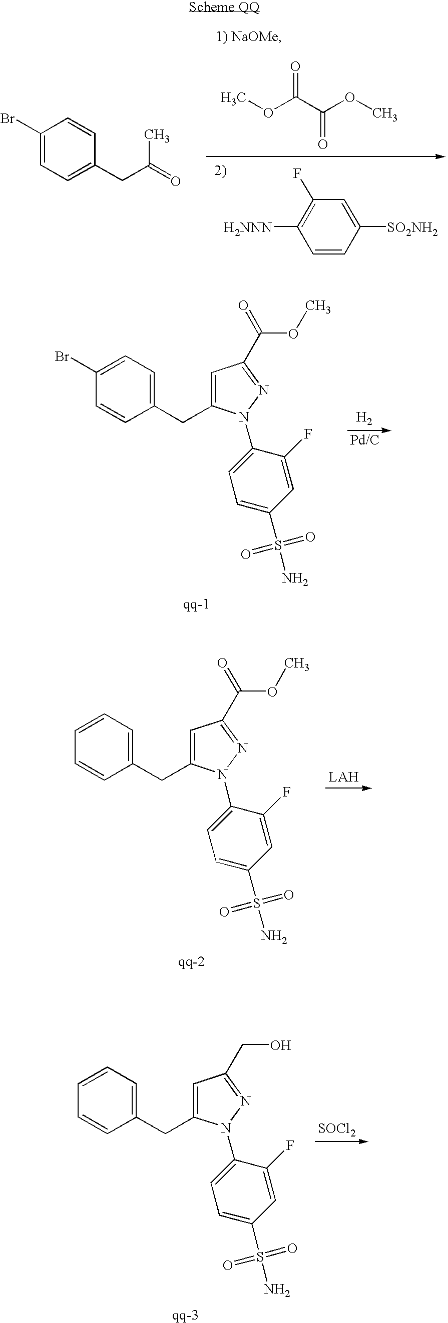

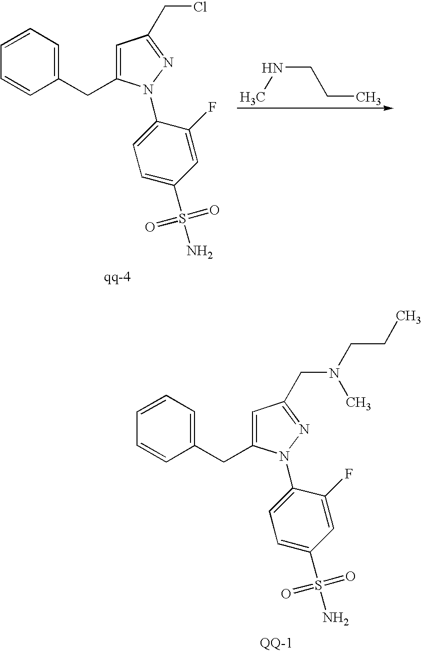

### Method QQ

### Example QQ-1

### 4-(5-benzyl-3-{[methyl(propyl)amino]methyl}-1H-pyrazol-1-yl)-3-fluorobenzenesulfonamide (QQ-1)

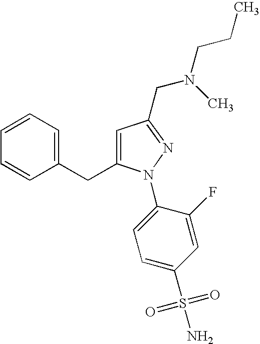

### Step 1: Methyl 1-[4-(aminosulfonyl)-2-fluorophenyl]-5-(4-bromobenzyl)-1H-pyrazole-3-carboxylate (qq-1)

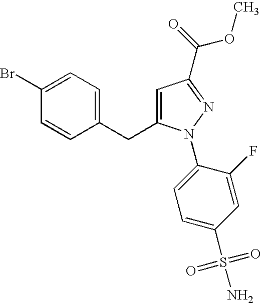

Obtained in the same fashion as that described in Method PP, step 1. 1-(4-Bromophenyl)acetone (15.0 g, 70.8 mmol), sodium methoxide (5.7 g), dimethyl oxalate (11.5 g) and 3-fluoro-4-hydrazinobenzenesulfonamide (12.3 g, 60 mmol, Pal, M.; et al J. Med. Chem. 2003; 46; 3975-3984) gave the title compound as a yellow solid (11.3 g, yield 34%), which was used without further purification. ¹H NMR (400 MHz, DMSO-d₆) 3.80 (s, 3H) 3.95 (s, 2H) 6.71 (s, 1H) 6.97 “(d, J=8.3 Hz, 2H) 7.42 (d, J=8.3 Hz, 2H) 7.70 (s, 2H) 7.79 (d, J=2.8 Hz, 2H) 7.83 (d, J=9.3 Hz, 1H).

### Step 2: Methyl 1-[4-(Aminosulfonyl)-2-fluorophenyl]-5-benzyl-1H-pyrazole-3-carboxylate (qq-2)

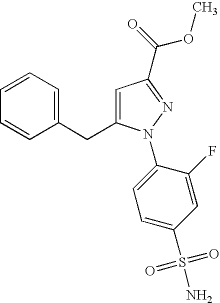

Methyl 1-[4-(aminosulfonyl)-2-fluorophenyl]-5-(4-bromobenzyl)-1H-pyrazole-3-carboxylate (qq-1, 13.2 g, 28.3 mmol) and 10% Pd—C (2 g) in MeOH (300 mL) was hydrogenated at 50 psi overnight. The mixture was filtered and washed with MeOH. The filtrate was concentrated and dried in vacuo to give the title compound as a white solid (9.41 g, yield 86%), which was used without further purification. ¹H NMR (400 MHz, DMSO-d₆) 3.80 (s, 3H) 3.96 (s, 2H) 6.70 (s, 1H) 8.99 (d, J=6.5 Hz, 2H) 7.16-7.25 (m, 3H) 7.70 (s, 2H) 7.76-7.85 (m, 3H)

### Step 3: 4-[5-Benzyl-3-(hydroxymethyl)-1H-pyrazol-1-yl]-3-fluorobenzenesulfonamide (qq-3)

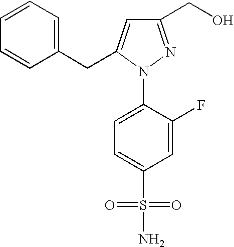

Methyl 1-[4-(aminosulfonyl)-2-fluorophenyl]-5-benzyl-1H-pyrazole-3-carboxylate (qq-2, 6.2 g, 15.9 mmol) was added portionwise into a suspension of LIAlH₄ (800 mg) in anhydrous THF (100 mL) under argon. The mixture was heated at 60° C. overnight. HPLC showed about 50% conversion, so another 470 mg of LIAlH₄ was added. The mixture was heated at 60° C. for 3 hours. The mixture was cooled to 0° C., sat. aq. Na₂SO₄ was added carefully to quench the reaction. The mixture was filtered through a patch of celite and washed with ethyl acetate. The filtrate was concentrated and extracted with 10% MeOH/CHCl₃, the organic layer was passed through a patch of Celite and dried over dry Na₂SO₄. The filtrate was evaporated to give a white solid (4.46 g, yield 78%), which was used without further purification. NMR provided in Table. Elemental Analysis: Calcd for C₁₇H₁₆FN₃O₃S.1.0 HCl: C, 51.32; H, 4.31; N, 10.56. Found: C, 51.48; H, 4.29; N, 10.50.

### Step 4: 4-[5-Benzyl-3-(chloromethyl)-1H-pyrazol-1-yl]-3-fluorobenzenesulfonamide (qq-4)

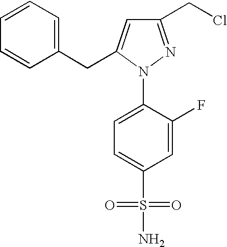

4-[5-Benzyl-3-(hydroxymethyl)-1H-pyrazol-1-yl]-3-fluorobenzenesulfonamide (qq-3, 1.0 g, 2.91 mmol) was placed under argon, thionyl chloride (5 mL) was added. The resultant solution was stirred for 1 hour. The residue was azeotroped with toluene to give 1.1 g of yellow powder which was used without further purification. ¹H NMR(300 MHz, DMSO-d₆) 3.95 (s, 2H) 4.73 (s, 2H) 6.32 (s, 1H) 7.02-7.07 (m, 2H) 7.19-7.30 (m, 3H) 7.69 (s, 2H) 7.74-7.79 (m, 2H) 7.83 (d, J=10.4 Hz, 1H)

### Step 5: 4-(5-Benzyl-3-{[methyl(propyl)amino]methyl}-1H-pyrazol-1-yl)-3-fluorobenzenesulfonamide Hydrochloride (QQ-1)

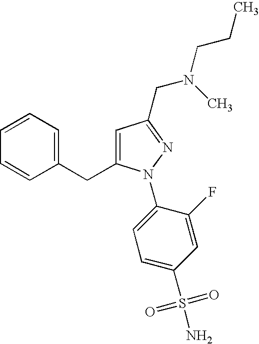

The title compound QQ-1 was made in same fashion as describe for Example F-1, step 4. 4-[5-Benzyl-3-(chloromethyl)-1H-pyrazol-1-yl]-3-fluorobenzenesulfonamide (qq-3; 200 mg, 0.53 mmol) and N-methyl-propylamine (116 mg, 1.59 mmol) after subsequently treatment with 3 N HCl in methanol gave the title compound QQ-1 as a white solid (0.46 mmol, yield 86%). NMR provided in table. Elemental Analysis: Calcd for C₂₁H₂₅FN₄O₂S.1.0 HCl: C, 55.68; H, 5.79; N, 12.37. Found: C, 55.60; H, 5.85; N, 12.22.

Examples qq-3a to qq-3d were prepared from the appropriate starting material in a manner analogous to the method of Example qq-3.

Examples QQ-1a to QQ-1o were prepared from the appropriate starting material in a manner analogous to the method of Example QQ-1.

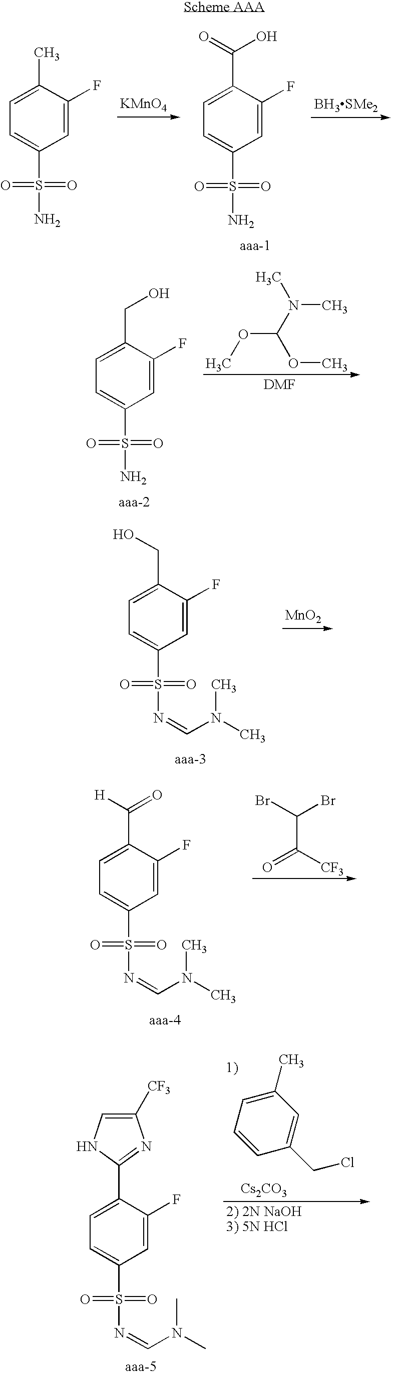

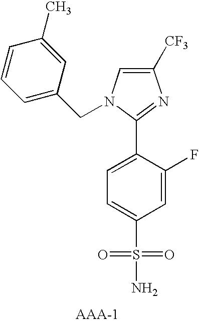

### Method AAA

### Example AAA-1

### 3-Fluoro-4-[1-(3-methylbenzyl)-4-(trifluoromethyl)-1H-imidazol-2-yl]benzenesulfonamide (AAA-1)

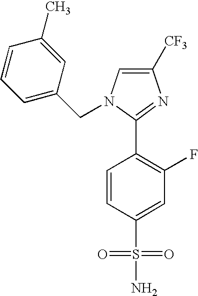

### Step 1: 4-(Aminosulfonyl)-2-fluorobenzoic Acid (aaa-1)

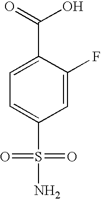

To a suspension of 2-flouro-4-toluenesulfonamide (1.00 g, 5.28 mmol) in water (50 mL) at reflux was added portionwise KMnO₄ (3.30 g) over 2 h, then allowed to cool and stir at ambient temperature for 48 h. The resultant suspension was filtered and the solid washed with water. The filtrate was acidified to pH 1 with concentrated HCl and extracted with EtOAc. The extract was dried over Na₂SO₄ and evaporated to dryness to give 1.00 g of a white solid, which was used without further purification. LCMS: M−1=218(30%), 174 (100%). ¹H NMR (400 MHz, CD₃OD) δ 7.69 (dd, J=10.07, 1.51 Hz, 1H), 7.75 (dd, J=8.18, 1.64 Hz, 1H), 8.07 (t, 1H).

### Step 2: 3-Fluoro-4-(hydroxymethyl)benzenesulfonamide (aaa-2)

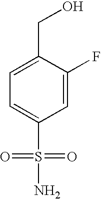

To a solution of crude 4-(aminosulfonyl)-2-fluorobenzoic acid, (1.00 g, 4.56 mmol) in THF (100 mL) was added BH₃.SMe₂ (2 mL). After stirring overnight at ambient temperature, methanol was carefully added portion-wise. The solvent was evaporated, resultant residue re-dissolved in MeOH/toluene, and concentrated to give 0.94 g (100%) of a white solid, which was used without further purification. LCMS (APCI, negative mode) 204. ¹H NMR (400 MHz, Acetone-d₆), δ 4.77 (s, 2H), 5.94 (bs, 3H), 7.56 (d, J=8.81 Hz, 1H), 7.69-7.77 (m, 2H).

### Step 3: N-[(Dimethylamino)methylene]-3-fluoro-4-(hydroxymethyl)benzenesulfonamide (aaa-3)

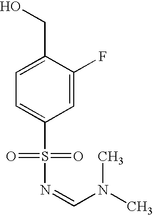

To 3-fluoro-4-(hydroxymethyl)benzenesulfonamide (1.01 g, 4.91 mmol) dissolved in minimal MeCN, was added N,N-dimethylformamide-dimethylformamide dimethyl acetal (1 mL). After 1 h at ambient temperature, the volatiles were removed to give a white solid (1.1 g, 86%), which was used without further purification. ¹H NMR (400 MHz, CDCl₃) δ 3.01 (s, 3H), 3.13 (s, 3H), 4.78 (s, 2H), 7.46-7.59 (m, 2H), 7.59-7.74 (m, 1H), 8.09 (s, 1H).

### Step 4: N-[(Dimethylamino)methylene]-3-fluoro-4-formylbenzenesulfonamide (aaa-4)

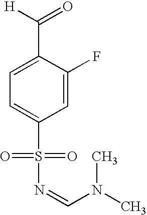

To a solution of N-[(dimethylamino)methylene]-3-fluoro-4-(hydroxymethyl)benzenesulfonamide (1.00 g, 3.84 mmol) in THF (50 mL), was added MnO₂ (3.30 g, 38.4 mmol). After 8 h stirring at ambient temperature, the mixture was filtered through a bed of Celite and the cake was washed with acetone. The filtrate was concentrated in vacuo to give a light yellow solid (950 mg, 96%), which was used without further purification. LCMS (APCI, pos Mode) M+1=259. ¹H NMR (400 MHz, acetone-d₆) δ 3.02 (s, 3H), 3.27 (s, 3H), 7.68 (dd, J=10.07, 1.51 Hz, 1H), 7.78 (dd, J=8:06, 1.26 Hz, 1H), 7.98 (dd, J=8.06, 6.80 Hz, 1H), 8.22 (s, 1H), 10.30-10.37 (m, 1H).

### Step 5: N-[(Dimethylamino)methylene]-3-fluoro-4-[4-(trifluoromethyl)-1H-imidazol-2-yl]benzenesulfonamide (aaa-5)

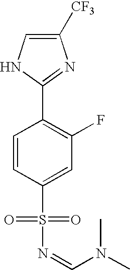

A solution of sodium acetate (2.71 g, 33.10 mmol) and 3,3-dibromo-1,1,1-triflouroacetone (5.95 g, 22.08 mmol) in water (50 mL) was heated at 100° C. for 1 h, then cooled to 0° C. with an ice bath. A solution of the aldehyde aaa-4 (950 mg, 3.68 mmol) in ethanol {20 mL) was added. 58% aq NH₄OH (6 mL) was added portionwise, maintaining the temperature below 5° C. The resultant mixture stirred at ambient temperature for 16 h. The solvent was removed in vacuo and the residue acidified with 2N HCl. The solution was washed with DCM (3×20 mL) and neutralized with NaHCO₃. The solid obtained was filtered and washed with water to give 1.0 g (71%) of the desired product, which was used without further purification. LCMS (APCI, Pos Mode) M+1=365.1 (100%). ¹H NMR (400 MHz, DMSO-d₆) δ ppm 2.93 (s, 3H), 3.16 (s, 3H), 7.64-7.80 (m, 2H), 7.97 (s, 1H), 8.13 (t, J=7.68 Hz, 1H), 8.26 (s, 1H), 12.59-13.45 (bs, 1H).

### Step 6: 3-Fluoro-4-[1-(3-methylbenzyl)-4-(trifluoromethyl)-1H-imidazol-2-yl]benzenesulfonamide (AAA-1)

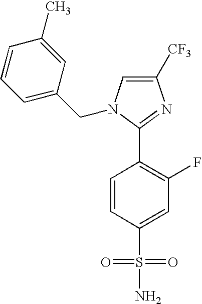

To a solution of the imidazole aaa-5 (1.16 mL of 0.25M in DMA) was added a solution of 3-methylbenzyl-chloride (0.58 mL of 0.5M in DMA, 2 equiv), of Cs₂CO₃ (265 mg, 0.823 mmol), and NaI (83 mg, 0.549 mmol). The resultant mixture stirred at 50° C. for 16 h, then 2N aq NaOH (10 mL) and ethanol (10 mL) were added. After another 2 h at 50° C., the mixture was allowed to cool to ambient temperature, and 5 N aq HCl (10 mL) was added. After 2 h, the volatiles were removed in vacuo and the residue was partitioned between saturated aqueous NaHCO₃ and EtOAc. The organic layer was dried over Na₂SO₄ and concentrated in vacuo. The product was purified using a solid phase extraction SCX column (Varian). The hydrochloride salt was obtained by dissolving the free-base in 5N HCl/Ethanol. After removing the volatiles, the residue was taken up in MeCN/water and freeze-dried to give the product as a white solid (65 mg, 60%). NMR provided in Table. The regiochemistry was confirmed with a NOESY 1H NMR experiment that indicated a relationship between the C12, C4 and C18 hydrogens, as depicted on the structure below:

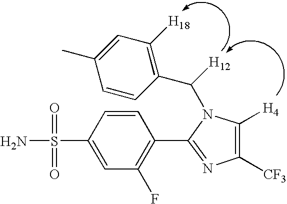

Example AAA-1a to 1m was prepared from the appropriate starting material in a manner analogous to the method of Example AAA-1.

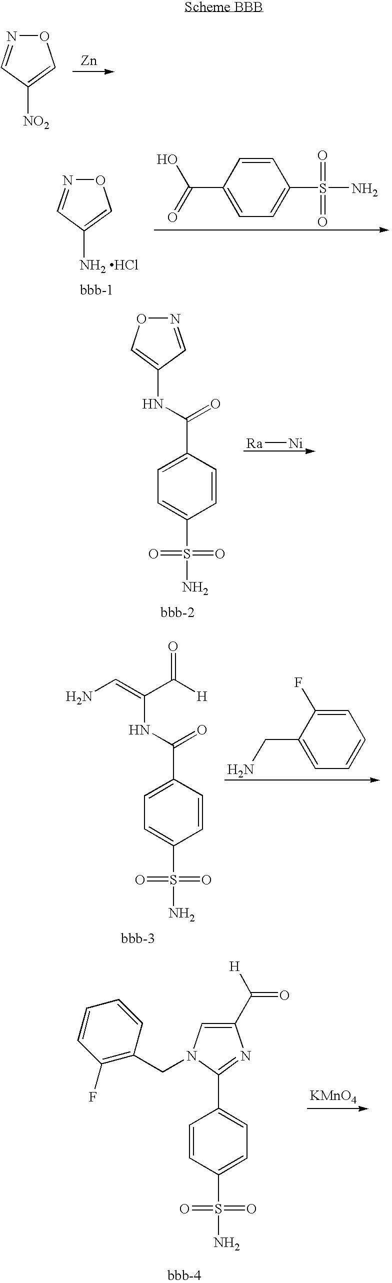

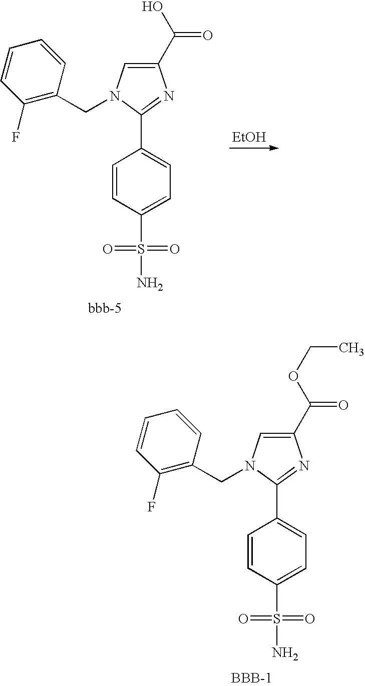

### Method BBB

### Example BBB-1

### Ethyl 2-[4-(Aminosulfonyl)phenyl]-1-(2-fluorobenzyl)-1H-imidazole-4-carboxylate (BBB-1)

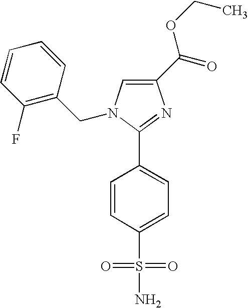

### Step 1: Isoxazol-4-amine Hydrochloride (bbb-1)

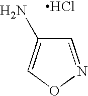

To a solution of NH4Cl (317 g, 5.93 mol) in water (1.4 L) was added 4-nitroisoxazole (30.9 g, 0.26 mol; 95% purity; Reiter, L. A.; J. Org. Chem.; 1987; 52; 2714-2726). The resultant suspension was cooled to 0° C., and Zn (143 g, 2.20 mol) added in portions (0.5-1 g) to keep the temperature below 5° C. (an ice bath). After about 40% of Zn was added, the exotherm apparently subsided. After the Zn addition was completed, the mixture was stirred at 5° C. for 10 h, the cooling bath was removed, allowed to warm to ambient temperature, and ethyl acetate (1750 mL) was carefully added dropwise under vigorous stirring. Then the mixture was filtered to remove zinc salts (Caution: leftover Zn reacted exothermically on the filter), and the organic layer was separated. The aqueous layer was extracted with -ethyl acetate. The combined organic layers were dried over MgSO4 and evaporated under reduced pressure to give an oily product (13.0 g), which was mixed with 4M HCl in dioxane (50 mL) and kept in a refrigerator overnight. The resultant precipitate was separated by filtration, washed with absolute ether (50 mL), and air-dried to give a light-brown solid in 48 yield % (15.1 g), which was used without further purification or characterization.

### Step 2: 4-(Aminosulfonyl)-N-isoxazol-4-ylbenzamide (bbb-2)

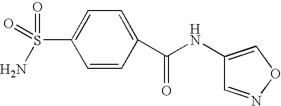

4-(Aminosulfonyl)benzoic acid (51.3 g, 0.25 mol) and acetonitrile (1 L) were placed into a 2 L three-necked flask equipped with a thermometer, reflux condenser with a calcium chloride tube, and a magnetic stirrer. After brief stirring, N,N′-carbonyldiimidazole (52.0 g, 0.32 mol) was added, and the mixture was stirred at 30-35° C. for 2 h. Crude isoxazol-4-amine hydrochloride (bbb-1; 33.1 g, 0.275 mol) was added. The resulting mixture was stirred at ambient temperature for 16-20 h. The solvent was rotary evaporated under reduced pressure. Acetone (100 mL) was added, and the mixture was filtered through a layer of silica gel with an addition of activated carbon. The filtrate was evaporated. Water (300 mL) was added to the residue. The suspension was heated at reflux for 30 min and filtered. The product was dried at 95° C. to give a solid (36.7 g, 55%), which was used without further purification. ¹H NMR (400 MHz, DMSO-d₆) δ 7.50 (s, 2H), 7.98 (d, J=8.6 Hz, 2H), 8.11 (d, J=8.6 Hz, 2H), 8.74 (s, 1H), 9.27 (s, 1H), 10.89 (s, 1H).

### Step 3: N-[2-Amino-1-formylvinyl]-4-(aminosulfonyl)benzamide (bbb-3)

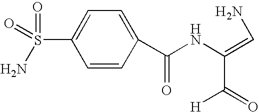

4-(Aminosulfonyl)-N-Isoxazol-4-ylbenzamide (bbb-2, 36.5 g, 0.136 mol), distilled DMF (400 mL), triethylamine (2 mL), and Raney nickel (30 g) were placed into a 1 L autoclave equipped with a magnetic stirrer. The mixture was stirred at ambient temperature and a hydrogen pressure of 20 atm for 10-12 h. The resultant solid was filtered off and washed with DMF. The filtrate was rotary evaporated under reduced pressure, and the resultant residue suspended in water (300 mL). The obtained suspension was heated at reflux for 30 min and allowed to cool. The resultant solid was filtered off, washed with water, and dried at 95° C. to afford a white solid (34.7 g, 95%), which was used immediately without further purification. ¹H NMR (400 MHz, DMSO-d₆) δ 6.93 (s, 1H), 7.22 (t, 1H), 7.34 (bs, 1H), 7.45 (s, 2H), 7.88 (d, J=8.31 Hz, 2H), 8.08 (d, J=8.56 Hz, 2H), 8.83 (s, 1H), 8.98 (bs, 1H).

### Step 4: 4-[1-(2-Fluorobenzyl)-4-formyl-1H-imidazol-2-yl]benzenesulfonamide (bbb-4)

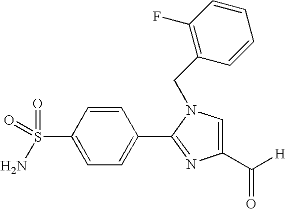

Aldehyde bbb-3 (4.0 g, 0.015 mol), distilled DMF (20 mL), methanol (100 mL), and (2-fluorobenzyl)amine (5.2 mL, 0.045 mol) were placed into a 1 L round-bottomed flask equipped with a reflux condenser. The suspension was heated at reflux until complete dissolution for 40-50 H). The solvents were distilled off in vacuo. The residue (9.7 g) was triturated with absolute ether. Ether was decanted, the residue was purified by chromatography with silica gel and eluted with ethyl acetate/benzene (4:1). The obtained oil was treated with ethyl acetate, and the resultant precipitate recrystallized from 70% aqueous acetic acid to give 370 mg of solid (7%).

¹NMR (400 MHz, DMSO-d₆) δ 5.50 (s, 2H), 6.97-7.09 (m, 1H), 7.10-7.23 (m, 2H), 7.30-7.41 (m, 1H), 7.48 (s, 2H), 7.77-7.87 (m, 2H), 7.87-7.96 (m, 2H), 8.19 (s, 1H), 9.79 (s, 1H).

### Step 5: 2-[4-(Aminosulfonyl)phenyl]-1-(2-fluorobenzyl)-1H-imidazole-4-carboxylic Acid (bbb-5)

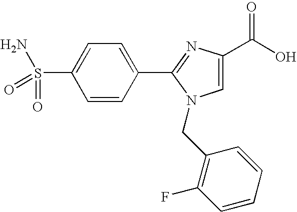

To a solution of 4-(1-(2-fluorobenzyl)-4-formyl-1H-imidazol-2-yl)benzenesulfonamide (bbb-4; 0.500 g, 1.39 mmol) in acetone (6 mL) and water (1 ml) was added potassium permanganate (0.297 g, 1.88 mmol) in small portions. The mixture stirred at ambient temperature for 18 h, then quenched with sodium thiosulfate (0.7 g, 2.78 mmol) and filtered through Celite. The filtrate was concentrated in vacuo and residue purified by reverse-phase HPLC using acetonitrile in water 45-95% gradient) to give the title compound 0.213 g (55%). HRMS calcd for C₁₇H₁₅FN₃O₄S⁺[M+H⁺]: 376.0762, found: 378.0775. ¹H NMR (300 MHz, DMSO-d₆) δ ppm 5.42 (s, 2H) 7.02 (t, J=7.54 Hz, 1H) 7.13-7.25 (m, 2H) 7.32-7.41 (m, 2H) 7.49 (br. s., 2H) 7.87 (s, 4H).

### Step 6: Ethyl 2-[4-(Aminosulfonyl)phenyl]-1-(2-fluorobenzyl)-1H-imidazole-4-carboxylate (BBB-1)

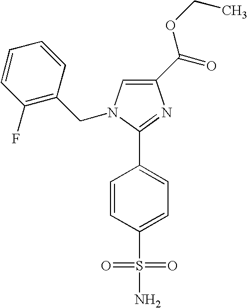

To a solution of 2-[4-(aminosulfonyl)phenyl]-1-(2-fluorobenzyl)-1H-imidazole-4-carboxylic acid (bbb-5; 0.05 g, 0.133 mmol) in ethanol (2 mL) was added DOWEX 50X8-200 resin (0.067 g, 0.03 mmol). The mixture was heated at 80° C. for 24 h. The mixture was filtered and purified by reverse phase HPLC using acetonitrile in water (5-95% gradient) to give the title compound 0.016 g (30%). NMR provided in Table. HRMS calcd for C₁₉H₁₉FN₃O₄S⁺[M+H⁺]: 404.1075, found: 404.1090.

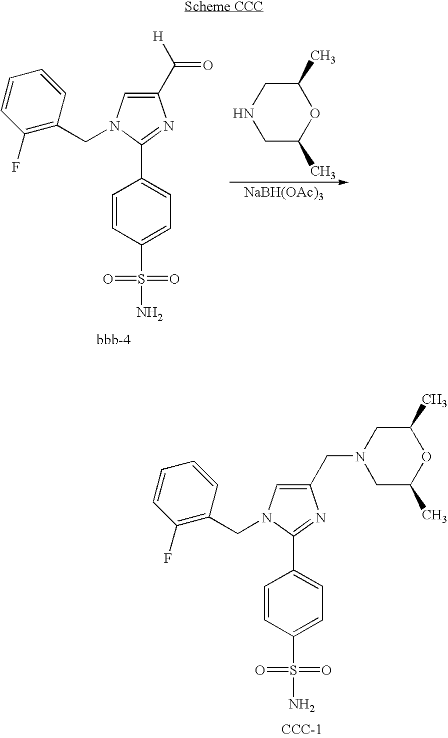

### Method CCC

### Example CCC-1

### 4-[4-{[(2R,6S)-2,6-Dimethylmorpholin-4-yl]methyl}-1-(2-fluorobenzyl)-1H-imidazol-2-yl]benzene-sulfonamide (CCC-1)

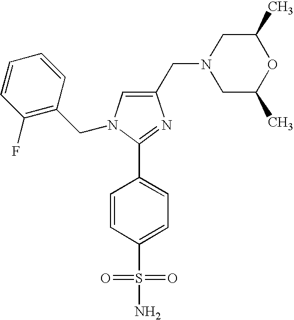

To a solution of the 4-(1-(2-fluorobenzyl)-4-formyl-1H-imidazol-2-yl)benzenesulfonamide (bbb-4, 100 mg, 0.27 mmol) in anhydrous MeCN (5 mL) was added a solution of 2,6-cis-dimethylmorpholine (31 mg, 0.27 mmol) in MeCN (2 mL). To this solution was added a suspension of NaBH(OAc)₃ (137 mg, 648 mmol) in MeCN (2 mL). The reactions were stirred for 8 h at room temperature. The excess hydride was quenched with 5% aqueous NaOH and the resultant mixture extracted with EtOAc. The organic layers were dried over Na2SO4 and loaded in to a pre-washed SOX cartridge (methanol, 2×5 mL). After washing with methanol, the product was eluted with 7 N NH₃/MeOH. After removing the volatiles via rotary evaporation under reduced pressure, the products were obtained as white solid (115 mg, 93% yield). NMR provided in Table. HRMS calcd for C23H28FN4O3S+[M+H+]: 459.1866, found: 459.1855.

Example CCC-1a and CCC-1b was prepared from the appropriate starting material in a manner analogous to the method of Example CCC-1.

### Methods R, RR, or RRR: Combinatorial Amide Coupling

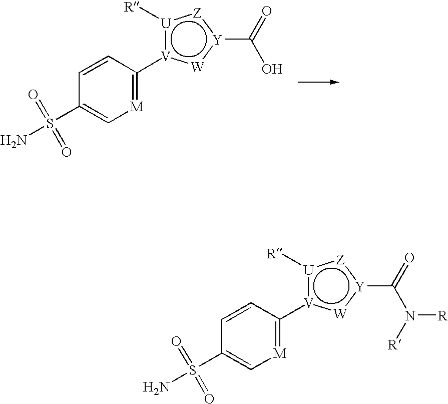

A stock solution of acid (560 uL of 0.125 M solution in anhydrous N,N-dimethylformamide, 0.07 mmol), the amine (350 uL of 0.20 M solution in anhydrous DMF, 0.070 mmol,), triethylamine (280 uL of 1.0 M in anhydrous DMF, 0.28 mmol), and O-(7-azabenzotriazol-1-yl)-N,N,N′,N′-tetra-methyluronium hexafluorophosphate (HATU; 140 uL of 0.5M in DMF, 0.07 mmol) were placed into a reaction tube. The reaction mixtures were stirred at 60° C. in a preheated AlaSyn™ reactor block for 18 h. The solvent was evaporated and the residue dissolved in DMSO (containing 0.01% 2,6-di-tert-butyl-4-methylphenol (BHT)) to give 0.0572 M solution (theoretical). The solution was injected into an automated chromatography system (specific gradient eluant, conditions listed in “Preparative Parameters”) and the fraction containing product was collected. The solvent was evaporated and the residue dissolved in the appropriate volume of DMSO to give an either 30 mM or 10 mM solution. The product solutions were analyzed by LCMS and submitted for screening.

### Methods S, SS, or SSS: Combinatorial Reductive Amination

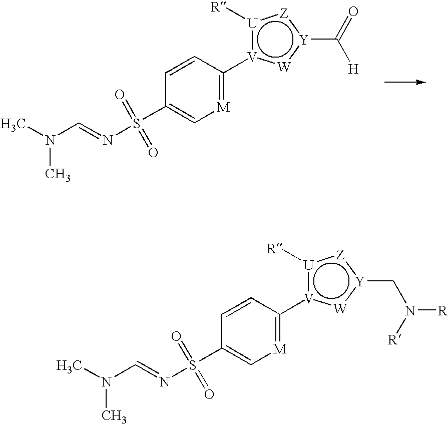

To a stock solution of aldehyde, either with (as depicted above), or in specific cases, without a N-(dimethylamino)methylene protected sulfonamide group (175 uL of 0.40 M in anhydrous acetonitrile, 0.070 mmol), was sequentially added amine (175 uL of 0.4 M in anhydrous acetonitrile, 0.07 mmol), sodium triacetoxyborohydride (210 uL of 0.800 M suspension in anhydrous acetonitrile, 0.168 mmol) were placed into the reaction test tube. The test tube rack was covered with Parafilm™, and agitated in a vortex shaker at ambient temperature overnight (16-24 h). The reaction was treated with 280 uL of H₂O and agitated in the vortex shaker at room temperature for 30 min, and then the solvents were evaporated. To the residue were added 560 μL of anhydrous ethanol and 700 μL of 0.5 M aqueous solution (0.35 mmol) of K₂CO₃ to each test tube. The reaction tubes were placed in an Alasyn™ reactor block that had been preheated to 50° C. and stirred for 4 h at that temperature. After cooling, 280 μL (0.7 mmol) of a 2.5 M aqueous HCl solution was added to each test tube in two portions of 140 μL each. The reaction rack was covered with a sheet of Parafilm™ and agitated on an orbit shaker for 2 h. The ethanol and water were removed in vacuo until all test tubes appear dry. The residue was dissolved in DMSO (containing 0.01% BHT) to give 0.0572 M solution (theoretical). The solution was injected into an automated chromatography system (specific gradient eluant, conditions listed in “Preparative Parameters”) and the fraction containing product was collected. The solvent was evaporated and the residue dissolved in the appropriate volume of DMSO to give an either 30 mM or 10 mM solution. The product solutions were analyzed by LCMS and submitted for screening.

### Method T or TT: Combinatorial Amine Alkylation

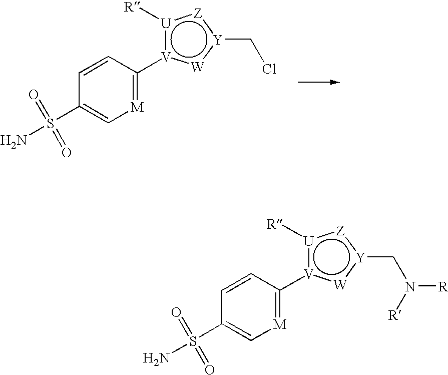

Anhydrous diisopropylethylamine (39 uL, 0.225 mmol), with 1) free amine monomer in N,N-dimethyl acetamide (563 uL of 0.4M, 0.225 mmol), or 2) for amine acid salt, the equal equivalents of diisopropylethylamine with the counter acid were added), and the corresponding 3-chloromethyl-azole analog in N,N-dimethyl acetamide solution (113 uL of 0.4M, 0.045 mmol) were added into the wells of a 2-mL polypropylene deep well plate. The plate was sealed in a Mat Cap Lined Plate Vise™ apparatus and heated overnight at 50° C. (16-24 h). After removing the solvents in vacuo, the residue was dissolved in DMSO (containing 0.01% BHT) to give 0.0572 M solution (theoretical). The solution was injected into an automated chromatography system (specific gradient eluant, conditions listed in “Preparative Parameters”) and the fraction containing product was collected. The solvent was evaporated and the residue dissolved in the appropriate volume of DMSO to give an either 30 mM or 10 mM solution. The product solutions were analyzed by LCMS and submitted for screening.

### Method JJJ: Combinatorial Regloselective 3-Trifluoromethyl-imidazole N1-Alkylation

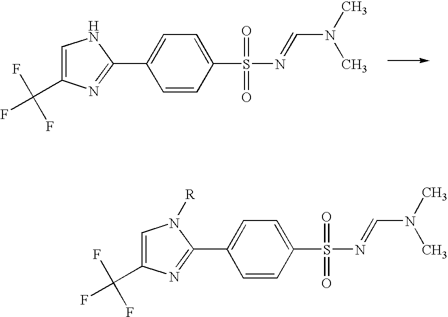

In a glove box under nitrogen was placed in a reaction tube approximately 69 mg of Cs₂CO₃, N-[(1E)-(dimethylamino)methylene]-4-[4-(trifluoromethyl)-1H-imidazol-2-yl]benzenesulfonamide stock solution in DMA (280 μL of 0.25M, 0.07 mmol), and to tubes with alkyl chloride monomer, approximately 10.5 mg (˜0.07 mmol) of NaI. Then an alkyl halide in N,N-dimethyl acetamide solution (140 μL, 0.07 mmol, 0.5M) was added to the reaction test tube. The reaction test tubes were placed in an Alasyn™ reactor block that had been preheated to 50° C. and stirred for 16 h at that temperature. Then the reaction tubes were centrifuged (no heat or vacuum). Approximately 350 μL of the supernatant solution was transferred from the reaction test tubes into the new set of clean test tubes. 175 μL of ethanol was dispensed to each original reaction test tube to extract the remaining contents. Again, the supernatant solution was transferred from the reaction test tubes into the corresponding new set of clean test tubes. To the residue was added 700 μL (0.35 mmol) of 0.5 M aqueous solution of K₂CO₃ to each test tube. The reaction tubes were placed in an Alasyn™ reactor block that had been preheated to 50° C. and stirred for 4 h at that temperature. After cooling, 140 μL of a 5 M (0.7 mmol) aqueous HCl solution was added to each test tube. The reaction rack was covered with a sheet of Parafilm™ and agitated on an orbit shaker for at least 2 h. The ethanol and water were removed in vacuo until all test tubes appeared dry. The residue was dissolved in DMSO (containing 0.01% BHT) to give 0.0572 M solution (theoretical). The solution was injected into an automated chromatography system (specific gradient eluant, conditions listed in “Preparative Parameters”) and the fraction containing product was collected. The solvent was evaporated and the residue dissolved in the appropriate volume of DMSO to give an either 30 mM or 10 mM solution, analyzed by LCMS, and submitted for screening.

The library was purified using columns with the gradient conditions listed in “Preparative Parameters.”

### Preparative Parameters for Purification

Purification Method
Preparative LC Method A (LC-A)
Preparative LC Method B (LC-B)
Preparative LC Method C (LC-C)
Preparative LC Method D (LC-D)
Preparative LC Method D (LC-D)
Preparative LC Method E (LC-E)
Preparative LC Method f (LC-f)
Preparative LC Method G (LC-G)
Preparative LC Method H (LC-H)
Preparative SFC Method A (SF-A)
Preparative SFC Method A (SF-A)
Preparative SFC Method A (SF-A)
Preparative SFC Method A (SF-A)
Preparative SFC Method B (SF-B)

HPLC instrument Components: Waters 2747 Sample Manager Autosampler, Waters 2757 Sample Manager Collector, Waters 2525. HPLC Pump, Waters Fluidics Organizer Column Selection Valve, Waters 515 HPLC Pump (for MS make up flow), Waters 2996 PDA Detector, Waters MicroMass ZQ Detector, SFC HPLC instrumentation, Berger PrepSFC™ semi-preparative purification system (Mettler-Toledo AutoChem, Inc.).

Preparative LC Method A (LC-A): Column: Waters Xterra C18, 19 mm×50 mm, 5 μm; Eluent A: 10.0 mM Ammonium Acetate in Water; Eluent B: Acetonitrile; Pre-inject Equilibration: ˜1.0 min, 5% B; Post-inject Hold: 0.9 min, 5% B at 40 mL/min; Gradient: 5-90% B in 2.55 minutes, hold 90% B for 0.2 min, then ramp 90% back to 5% in 0.1 min; Flow: 50.0 mL/min; Column Temp: Ambient; Injection Amount: 1200 μL of flttered crude reaction mixture in DMSO; Detection: ESI positive mode, 1:10000 split from flow, MeOH-carrier.

Preparative LC Method B (LC-B): Column: Waters Xterra C18, 19 mm×50 mm, 5 μm; Eluent A: 0.05% TFA in Water, Eluent B: 0.05% TFA in Acetonitrile; Pre-inject Equilibration: ˜1.0 min, 5% B; Post-inject Hold: 0.9 min, 5% B at 40 mL/min; Gradient 5-90% B in 2.55 minutes, hold 90% B for 0.2 min, then ramp 90% back to 5% in 0.1 min; Flow: 50.0 mL/min at gradient start; Column Temp: Ambient; Injection Amount: 1200 μL of filtered crude reaction mixture in DMSO; Detection: ESI positive mode, 1:10000 split from flow, MeOH carrier.

Preparative LC Method C (LC-C): Column: Phenomenex Gemini C18, 21.2 mm×50 mm, 5 μm; Eluent A: 0.1% Ammonium Hydroxide in Water, Eluent B: Acetonitrile; Pre-inject Equilibration: −1.0 min, 5% B; Post-inject Hold: 0.9 min, 5% B at 40 mL/min; Gradient: 5-90% B in 2.55 minutes, hold 90% B for 0.2 min, then ramp 90% back to 5% in 0.1 min; Flow: 50.0 mL/min at gradient start; Column Temp: Ambient; Injection Amount: 1200 μL of filtered crude reaction mixture in DMSO; Detection: ESI positive mode, 1:10000 split from flow, MeOH carrier.

Preparative LC Method D (LC-D): Column: Phenomenex Gemini C18, 21.2 mm×50 mm, 5 μm; Eluent A: 10.0 mM Ammonium Acetate in Water, Eluent B: Acetonitrile; Pre-inject Equilibration: −1.0 min, 5% B; Post-inject Hold: 0.9 min, 5% B at 40 mL/min; Gradient 5-90% B in 2.55 minutes, hold 90% B for 0.2 min, then ramp 90% back to 5% in 0.1 min; Flow: 50.0 mL/min at gradient start; Column Temp: Ambient; Injection Amount: 1200 μL of filtered crude reaction mixture in DMSO; Detection: ESI positive mode, 1:10000 split from flow, MeOH carrier.

Preparative LC Method E (LC-E): Column: Chromolith prep RP-18e, 20 mm×100 mm; Eluent A: 10.0 mM Ammonium Acetate in Water, Eluent B: Acetonitrile; Pre-inject Equilibration: −1.0 min, 10% B; Post-inject Hold: 0.9 min, 10% B at 40mL/min; Gradient: 10-90% B in 3.40 minutes, hold 90% B for 0.40 min, then ramp 90% back to 5% in 0.1 min; Flow: 50.0 mL/min at gradient start; Column Temp: Ambient; Injection Amount 1200 μL of filtered crude reaction mixture in DMSO; Detection: ESI positive mode, 1:10000 split from flow, MeOH carrier.

Preparative LC Method F (LC-F): Column: Phenomenex Gemini AXIA C18, 21.2 mm×50 mm, 5 μm; Eluent A: 10.0 mM Ammonium Acetate in Water; Eluent B: Acetonitrile; Pre-inject Equilibration: ˜1.0 min, 5% B; Post-inject Hold: 0.9 min, 5% B at 40 mL/min; Gradient: 5-90% B in 2.55 minutes, hold 90% B for 0.2 min, then ramp 90% back to 5% in 0.1 min; Flow: 50.0 mL/min at gradient start; Column Temp: Ambient; Injection Amount: 1200 μL of filtered crude reaction mixture in DMSO; Detection: ESI positive mode, 1:10000 split from flow, MeOH carrier.

Preparative LC Method G (LC-G): Column: Peeke Scientific Ultro 60 C18 5 μm, 50 mm×20 mm; Eluent A: Water/0.05% TFA; Eluent B: Acetonitrile/0.05% TFA; Pre-inject Equilibration: 10% B; Post-inject Hold: 0.15 min, 10% B at 25 mL/min; Gradient: 10-95% B between 0.15 and 5 min, hold 95% 8 for 1.5 min, then ramp 95% back to 10% in 0.25 min, then hold 10% B for 0.5 min; Flow: 25.0 mL/min; Column Temp: 41° C.; Injection Amount: 2500 μL of filtered crude reaction mixture in DMSO; Detection: UV 200-400 nm, ELSD

Preparative LC Method H (LC-H); Column: Phenomenex Gemini C18 5μ 10 A 50×21.2 mm; Eluent A: Water/0.05% TFA; Eluent B: Acetonitrile/0.05% TFA; Pre-inject Equilibration: 5% B; Post-inject Hold: 0.2 min, 5% 8 at 25 mL/min; Gradient: 5-75% B between 0.2 and 5 min, hold 95% B for 1 min, then ramp 95% back to 5% in 0.25 min, then hold 5% B for 0.15 min; Flow: 25.0 ml/min; Column Temp: 41° C.; Injection Amount: 2000 μL of filtered crude reaction mixture in DMSO; Detection: UV 200-400 nm, ELSD.

Preparative SFC Method A (SF-A): Column: ZymorSPHER Diol 21.2 mm×150 mm, 100 A, 5 μm; Eluent A: CO2; Eluent B: MeOH w/0.1% isopropylamine; Pre-inject Equilibration: ˜1.0 min, 5% B Post-inject Hold: none; Gradient: 5-50% B ramp at 9%/min, hold 50% B for 0.5 min, then ramp 50% back to 5% at 99%/min; Flow: ˜62.0 mL/min; Pressure: 140 bar; Column Temp: 35° C.; Nozzle (BPR) Temp: 40° C.; Injection Amount 1200 μL of filtered crude reaction mixture in DMSO; Detection: UV 260 nm.

Preparative SFC Method B (SF-B): Column: ZymorSPHER Pyr/Diol 21.2 mm×150 mm, 100 A, 5 μm; Eluent A: CO2; Eluent B: MeOH; Pre-inject Equilibration: ˜1.0 min, 5% B; Post-inject Hold: none; Gradient: 5-50% B ramp at 9%/min, hold 50% B for 0.5 min, then ramp 50% back to 5% at 99%/min; Flow: ˜62.0 mL/min; Pressure: 140 bar; Column Temp: 35° C.; Nozzle (BPR) Temp: 40° C.; Injection Amount 1200 μL of filtered crude reaction mixture in DMSO; Detection: UV 260 nm.

Preparative SFC Method C (SF-C): Column: Zymor Pegasus 21.2 mm×150 mm, 100 A, 5 μm; Eluent A: CO2; Eluent B: MeOH; Pre-inject Equilibration: ˜1.0 min, 5% B; Post-inject Hold: none; Gradient: 5-50% B ramp at 9%/min, hold 50% B for 0.5 min: then ramp 50% back to 5% at 99%/min; Flow: ˜62.0 mL/min; Pressure: 140 bar; Column Temp: 35° C.; Nozzle (BPR) Temp: 40° C.; Injection Amount 1200 μL of filtered crude reaction mixture in DMSO; Detection: UV 260 nm.

Carbonic Anhydrase-II (CA-II) Fluorimetric Assay-IC₅₀ Determination

Compounds were diluted in DMSO at the concentration of 1 mM, then 50 μM and transferred to a 96-well plate for further dilutions (1:3 dilution, 11 points) in duplicate. Highest final concentration of compound in this CA-II Fluorimetric assay is 1 μM. Assays were conducted in a final volume of 100 μL in 50 mM Tris/HCl (pH 7.6), 100 mM Na2SO4 and 0.005% Tween-20 in a 96-well black assay plate. Fluorescein diacetate was used as the substrate. Enzyme inhibition was determined by pipetting 8 μL of human CA-II (5 nM, from Sigma-Aldrich, product #: C6165) into assay plate contained 2 μL of compound and 2 μL of substrate (10 μM) in 88 μL of assay buffer. The rate of the hydrolysis of fluorescein diacetate were measured spectrophometrically at 488 nm (excitation), 538 nm (emission) and 530 nm (cutoff) using a Molecular Devices SpectraMax M2 fluorescence reader at 25° C. The IC50, the inhibitor concentration resulting in 50% inhibition of the enzyme activity, was calculated using GraphPad Prism or similar in-house software with the IC50 curve fitting using the four parameter logistic equation.

Carbonic Anhydrase-II (CA-II) Fluorescence Polarization Tight Binding Assay-Kd Determination for compounds with IC₅₀≦2.5 nM

Compounds were diluted in DMSO at the concentration of 1 mM, 50 uM then to 0.5 uM and transferred to a 96-well plate for further dilutions (1:1.353 dilution, 11 points) in quadruple. Final compound highest concentration in CA-II Kd FP Tight Binding Assay is 0.01 uM. Assays were conducted in a final volume of 100 μL in 50 mM Tris/HCl (pH 7.6), 100 mM Na2SO4 and 0.005% Tween-20 in a 96-well black assay plate. Dye conjugate BODIPY®558/588-Acetazolamide (Invitrogen Corp.) was used as the tracer. Binding inhibition was determined by pipetting 8 μL of human CA-II (1.5 nM) into assay plate contained 2 μL of compound and 2 μL of tracer (2 nM) in 88 μL of assay buffer. The assay plate was

Incubated at room temperature for 1 hour and read in the fluorescence polarization reader (Molecular Devices, Analyst) at 524/45 nm (excitation), 595/60 nm (emission) and 561 nm (beam splitter). The Kd binding was calculated using GraphPad Prism and Morrison tight binding ligand equation.

Carbonic Anhydrase-IV (CA-IV) Fluorescence Polarization Assay-IC₅₀ Determination

Human CAIV was amplified from a human kidney cDNA library (Clonetech) using primers: 5′-ggaattccatatggcagagtcacactggtgctacgag and 5′-ccgctcgagttactaggactttatcaccgtgcgctgccc, with KOD Polymerase (Novagen). The PCR amplified product was cloned into a NdeI/XhoI cut pET-43.1a(+) (Novagen) and transformed into Escherichia coli BL21.(DE3) (Invitrogen) cells. These cells were grown in Luria broth (LB) media (Biomyx) supplemented with 800 uM ZnCl₂ at 37° C. until an O.D.600 of 0.7, at which point the cells were induced with 100 uM isopropyl-beta-D-thiogalactopyranoside (IPTG) for 20 hours at 20° C. The frozen pellet was resuspended in 50mM 2-morpholinoethanesulfonic acid (MES) at pH 6.0, 100 mM NaCl, 800 uM ZnCl₂ and EDTA-Free protease inhibitors (Roche). The cells were lysed with a microfluidizer, the lysate was spun at 40,000 rpm for 45 minutes at 4° C., and the soluble fraction was dialyzed overnight at 4° C. in 50 mM MES pH 6.0, and 100 mM NaCl. The soluble fraction was then put over a 100 ml SP-Sepharose High Performance (GE Healthcare) column and eluted with a 50 mM MES pH 6.0, 750 mM NaCl gradient. The peak fractions were then concentrated, via an Amicon Ultra-4 10,000 MWCO (Millipore) spin column to 2.0 mL and loaded onto a Sephacryl S-100 High Resolution (GE Healthcare) column in 25 mM tris(hydroxymethyl)aminomethane hydrochloride (Tris-HCl) pH 7.0, and 100 mM NaCl. The peak fractions were then concentrated, via an Amicon Ultra-4 10,000 MWCO (Millipore) spin column, to 7.0 mg/mL and left exposed at room temperature overnight. Fully-oxidized protein was characterized by performing an Ellman's Assay, utilizing Pierce reagents, and non-reducing SDS-PAGE. The specific activity was confirmed with literature inhibitors (IC₅₀ for acetazolamide (120 nM), ethoxzolamide (88 nM), dorzolamide (43 nM) and brinzolamide (45 nM); as reported in Innocenti, A.; et al, Bioorg. Med. Chem. Lett. 2005, 15, 1149-1154 and Supuran, C. T.; et al. Carbonic Anhydrase Inhibitors. Med. Res. Rev. 2003, 23, 146-189).

Carbonic Anhydrase-IX (CA-IX) Fluorescence Polarization Assay-IC₅₀ Determination

Compounds were diluted in DMSO at the concentration of 1 mM, then to 250 μM and transferred to a 96-well plate for further dilutions (1:3 dilution, 11 points) in duplicate final compound highest concentration in CA-IV FP assay is 5 uM. Assays were conducted in a final volume of 100 μL in 50 mM Tris/HCl (pH 7.6), 100 mM Na₂SO₄ and 0.005% Tween-20 in a 96-well black assay plate. BODIPY6558/568-Acetazolamide was used as the tracer. Binding inhibition was determined by pipetting 8 μL of human CA-IV (25 nM) into assay plate contained 2 μL of compound and 2 μL of tracer(2 nM) in 88 μL of assay buffer. The assay plate was incubated at room temperature for 30 minutes and read in the fluorescence polarization reader (Molecular Devices, Analyst) at 524/45 nm (excitation), 595/60 nm (emission) and 561 nm (beam splitter). IC₅₀, the Inhibitor -concentration resulting in 50% inhibition of the enzyme activity was calculated using GraphPad Prism or similar in-house software with the IC₅₀ curve fitting using the four parameter logistic equation.

Carbonic Anhydrase-IX (CA-IX) Fluorescence Polarization Assay-IC₅₀ Determination

Compounds were diluted in DMSO at the concentration of 1 mM, then 50 μM and transferred to a 96-well plate for further dilutions (1:3 dilution, 11 points) in duplicate. Final compound highest concentration in CA-IX FP assay is 1 μM. Assays were conducted in a final volume of 100 μL in 50 mM Tris/HCl (pH 7.6), 100 mM Na2SO4 and 0.005% Tween-20 in a 96-well black assay. plate. BODIPY8558/568-Acetazolamide was used as the tracer. Binding inhibition was determined by pipetting 8 μL of human CA-IX (8 nM, R&D Systems Inc. Cat#2188-CA-010) into assay plate contained 2 μL of compound and 2 μL of tracer (2 nM) in 88 μL of assay buffer. The assay plate was incubated at room temperature for 30 minutes and read in the fluorescence polarization reader (Molecular Devices, Analyst) at 524/45 nm (excitation), 595/60 nm (emission) and 561 nm (bean splitter). IC50, the inhibitor concentration resulting in 50% inhibition of the enzyme activity was calculated using. GraphPad Prism or similar in-house software with the IC50 curve fitting using the four parameter logistic equation.

Carbonic Anhydrase-XII (CA-XII) Fluorimetric Assay-IC₅₀ Determination

Human CAXII was amplified from a human kidney cDNA library (Clonetech) using primers: 5′-ggaattccatatgaagtggacttattttggtcctgat and 5′-cccaagcttttactaggagaaggaggtgtataccagcct, with KOD Polymerase (Novagen). The PCR amplified product was cloned into a NdeI/HindIII cut pET-43.1a(+) and transformed into Escherichia coli AD494 (DE3) (Novegen) cells. The cells were grown in LB (Biomyx) supplemented with 800 uM ZnCl₂ at 37° C. until an O.D.600 of 0.7, at which point the cells were induced with 100 uM IPTG for 20 hours at 20° C. The frozen pellet was resuspended in 50 mM MES pH 6.0, 100 mM NaCl, 800 uM ZnCl₂ and EDTA-Free protease inhibitors (Roche). The cells were lysed with a microfluidizer and the lysate was spun at 40,000 rpm for 45 minutes at 4° C. The soluble fraction was then put over a 100 ml SP-Sepharose High Performance (GE Healthcare) column and eluted with a 50 mM MES pH 6.0, 750 mM NaCl gradient. The peak fractions were then dialyzed overnight in 10 mM Tris-SO₄ pH 7.3, concentrated, via an Amicon Ultra-4 10,000 MWCO (Millipore) spin column, to 7.0 mg/ml and characterized by non-reducing SDS-PAGE. The specific activity was confirmed with literature inhibitors (IC₅₀ for acetazolamide (42 nM), ethoxzolamide (17 nM), dorzolamide (14 nM) and brinzolamide (17 nM), as reported in Vullo, D.; et al. Bioorg. Med. Chem. Lett. 2005, 15, 963-969).

Compounds were diluted in DMSO at the concentration of 1 mM, then 50 μM and transferred to a 96-well plate for further dilutions (1:3 dilution, 11 points) in duplicate. Final compound highest concentration in CA-XII Fluorimetric assay is 1 μM. Assays were conducted in a final volume of 100 μL in 50 mM Tris/HCl (pH 7.6), 100 mM Na₂SO₄ and 0.005% Tween-20 in a 96-well black assay plate. Fluorescein diacetate was used as the substrate. Enzyme inhibition was determined by pipetting 8 μL of human CA-XII (50 nM) into assay plate contained 2 μL of compound and 2 μL of substrate (10 μM) in 88 μL of assay buffer. The rate of the hydrolysis of fluorescein diacetate were measured spectrophometrically at 488 nm (excitation), 538 nm (emission) and 530 nm (cutoff) using a Molecular Devices SpectraMax M2 fluorescence reader at 25° C. IC₅₀, the inhibitor concentration resulting in 50% inhibition of the enzyme activity was calculated using GraphPad Prism or similar in-house software with the IC₅₀ curve fitting using the four parameter logistic equation.

| | | | LRMS | | | | | |
|---|---|---|---|---|---|---|---|---|
| Example | | Chemical | m/z | | IC50_CAI | Kd_CAII | IC₅₀—CAII | IC50_CAIV |
| Number | Structure | Name | (M + H)+ | ¹H NMR | (nM) | (nM) | (nM) | (nM) |
| A-1 | 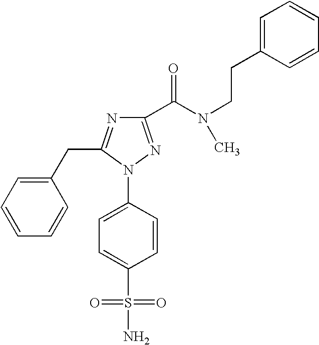 | 1-[4- (aminosulfonyl)phenyl]- 5-benzyl-N-methyl-N- (2-phenylethyl)-1H- 1,2,4-triazole-3- carboxamide | 476 | ¹H NMR (300 MHz, DMSO- d₆) 4.33 (s, 2 H) 7.03-7.07 (m,1 H) 7.13- 7.35 (m, 9 H) 7.57 (s, 2 H) 7.77-7.84 (m, 2 H) 7.98-8.05 (m, 2 H) | | 1.49 | 123 | 0.089 |
| A- 1a | 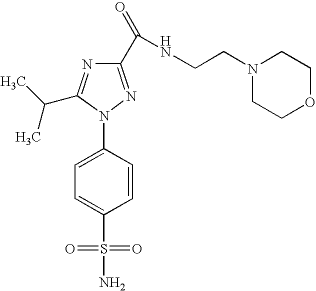 | 1-[4- (aminosulfonyl)phenyl]- 5-isopropyl-N-(2- morpholin-4-ylethyl)- 1H-1,2,4-triazole-3- carboxamide | 423 | ¹H NMR (300 MHz, DMSO- d₆) 1.27 (d, J = 6.8 Hz, 6 H) 3.04-3.27 (m, 3 H) 3.26-3.39 (m, 2 H) 3.71- 3.88 (m, 4 H) 3.99 (d, 2 H) 7.64 (s, 2 H) 7.84 (d, J = 8.5 Hz, 2 H) 8.07 (d, J = 8.5 Hz, 2 H) 8.88 (t, J = 5.9 Hz, 1 H), 10.85 (s, 1H) | | 2.60 | | 1.79 |
| A- 1b | 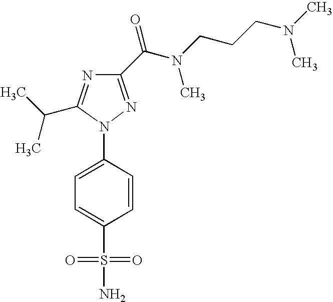 | 1-[4- (aminosulfonyl)phenyl]- N-[3- (dimethylamino)propyl]- 5-isopropyl-N- methyl-1H-1,2,4- triazole-3- carboxamide | 409 | ¹H NMR (300 MHz, DMSO- d₆) 1.26 (dd, J = 6.8, 2.3 Hz, 6 H) 1.93-2.15 (m, 2 H) 2.70 (d, J = 4.9 Hz, 3 H) 2.77 (d, J = 4.9 Hz, 3 H) 2.95-3.31 (m, 6 H) 3.55 (t, J = 7.06 Hz, 2 H) 7.62 (s, 2 H) 7.84 (d, J = 8.7 Hz, 1 H) 7.89 (d, J = 8.7 Hz, 1 H) 8.06(d, J = 8.7 Hz, 2 H) | | 6.41 | | |
| A- 1c | 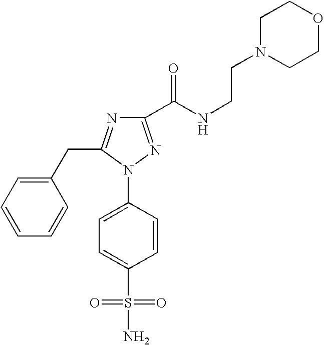 | 1-[4- (aminosulfonyl)phenyl]- 5-benzyl-N-(2- morpholin-4-ylethyl)- 1H-1,2,4-triazole-3- carboxamide | 471 | ¹H NMR (400 MHz, MeOD) 2.53-2.60 (m, 4 H) 2.63 (t, J = 6.6 Hz, 2 H) 3.59 (t, J = 6.5 Hz, 2 H) 3.72 (m, 4 H) 4.33 (s, 2 H) 7.08- 7.14 (m, 2 H) 7.20-7.31 (m, 3 H) 7.67 (d, J = 8.7 Hz, 2 H) 8.05 (d, J = 8.7 Hz, 2 H) | | 1.43 | 608 | 0.109 |
| A- 1d | 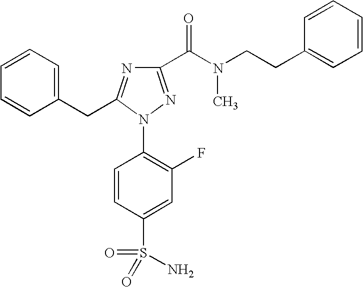 | 1-[4-(aminosulfonyl)-2- fluorophenyl]-5- benzyl-N-methyl-N-(2- phenylethyl)-1H-1,2,4- triazole-3- carboxamide | 494 | ¹H NMR (400 MHz, DMSO- d₆) 2.75-2.85 (m, 2 H) 3.54- 3.62 (m, 2 H) 4.07-4.10 (s, 2 H) 6.93-7.03 (m, 3 H) 7.08- 7.27 (m, 7 H) 7.65 (s, 2 H) 7.73-7.88 (m, 3 H) | | 2.08 | 148 | 0.216 |
| A- 1e | 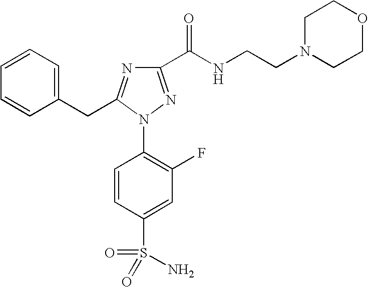 | 1-[4-(aminosulfonyl)-2- fluorophenyl]-5- benzyl-N-(2- morpholin-4-ylethyl)- 1H-1,2,4-triazole-3- carboxamide | 489 | ¹H NMR (400 MHz, MeOD) d 2.56 (t, J = 4.6 Hz, 4 H) 2.63 (t, J = 6.5 Hz, 2 H) 3.58 (t, J = 6.5 Hz, 2 H) 3.72 (t, J = 4.6 Hz, 4 H) 4.21 (s, 2 H) 7.03 (dd, J = 7.6, 2.1 Hz, 2 H) 7.18- 7.25 (m, 3 H) 7.64 (t, J = 7.6 Hz, 1 H) 7.84 (d, J = 8.3 Hz, 2 H) | | 2.26 | 562 | 0.116 |
| a-4 | 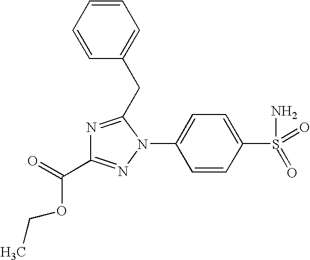 | ethyl 1-[4- (aminosulfonyl)phenyl]- 5-benzyl-1H-1,2,4- triazole-3-carboxylate | 387 | ¹H NMR (300 MHz, DMSO- d₆) 1.33 (t, J = 7.1 Hz, 3 H) 4.32 (s, 2 H) 4.37 (q, J = 7. Hz, 2 H) 7.14 (d, J = 7.2 Hz, 2 H) 7.21-7.35 (m, 3 H) 7.60 (s, 2 H) 7.83 (d, J = 8.7 Hz, 2 H) 8.01 (d, J = 8.7 Hz, 2 H) | | 1.68 | 556 | 0.111 |
| a- 4a | 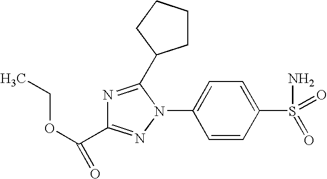 | ethyl 1-[4- (aminosulfonyl)phenyl]- 5-cyclopentyl-1H- 1,2,4-triazole-3- carboxylate | 365 | ¹H NMR (300 MHz, DMSO- d₆) d 1.33 (t, J = 7.1 Hz, 3 H) 1.62-1.68 (m, 2 H) 1.71-1.89 (m, 4 H) 1.89- 2.05 (m, 2 H) 3.14-3.33 (m, 1 H) 4.37 (q, J = 7.1 Hz, 2 H) 7.84 (d, J = 8.6 Hz, 2 H) 8.05 (d, J = 8.6 Hz, 2 H) | | 1.60 | 2650 | 0.75 |
| a- 4b | 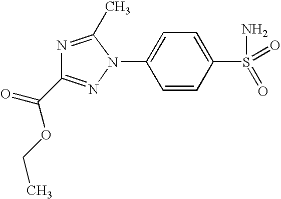 | ethyl 1-[4- (aminosulfonyl)phenyl]- 5-methyl-1H-1,2,4- triazole-3-carboxylate | 311 | ¹H NMR (300 MHz, DMSO- d₆) 1.34 (t, J = 7.1 Hz, 3 H) 2.58 (s, 3 H) 4.38 (q, J = 7.1 Hz, 2 H) 7.60 (s, 2 H) 7.90 (d, J = 8.9 Hz, 2 H) 8.04 (d, J = 8.9 Hz,2 H) | | 2.60 | 1400 | 0.232 |
| A- 4c | 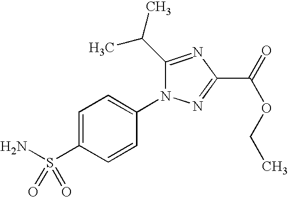 | ethyl 1-[4- (aminosulfonyl)-2- fluorophenyl]- 5-isopropyl-1H-1,2,4- triazole-3-carboxylate | 357 | ¹H NMR (300 MHz, DMSO- d6) 1.27 (d, J = 6.97 Hz, 6 H) 1.38 (t, J = 7.06 Hz, 3 H) 2.96-3.05 (m, 1 H) 4.42 (q, J = 7.10 Hz, 2 H) 7.78 (br. s., 2 H) 7.78 (br. s., 2 H) 7.93- 8.11 (m, 3 H) | | 3.30 | 5000 | |
| a- 4d | 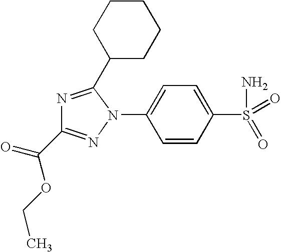 | ethyl 1-[4- (aminosulfonyl)phenyl]- 5-cyclohexyl-1H- 1,2,4-triazole-3- carboxylate | 379 | ¹H NMR (300 MHz, DMSO- d₆) 1.15-1.29 (m, 3 H) 1.33 (t, J = 7.0 Hz,3 H) 1.48-1.69 (m, 3 H) 1.68- 1.80 (m,2 H) 1.80-1.93 (m, 2 H) 2.77-2.94 (m,1 H) 4.37 (q, J = 7.0 Hz, 2 H) 7.62 (s,2 H) 7.84 (d, J = 8.1 Hz, 2 H) 8.06 (d, J = 8.1 Hz, 2 H) | | 2.17 | 1260 | 0.046 |
| A- 4e | 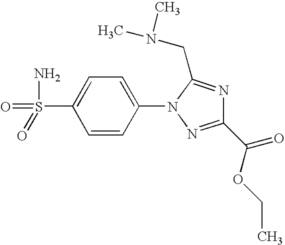 | ethyl 1-[4- (aminosulfonyl)phenyl]- 5- [(dimethylamino) methyl]-1H-1,2,4-triazole-3- carboxylate | 354 | ¹H NMR (300 MHz, DMSO- d₆) 1.36 (t, J = 7.1 Hz, 3 H) 2.83 (s, 6 H) 4.43 (q, J = 7.1 Hz, 2 H) 4.68 (s, 2 H) 7.66 (s, 2 H) 7.90 (d, J = 8.7 Hz, 2 H) 8.08 (d, J = 8.7 Hz, 2 H) | | 3.80 | 4050 | |
| A-4f | 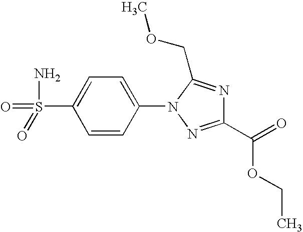 | ethyl 1-[4- (aminosulfonyl)phenyl]- 5-(methoxymethyl)- 1H-1,2,4-triazole-3- carboxylate | 341 | ¹H NMR (300 MHz, DMSO- d₆) 1.35 (t, J = 7.1 Hz, 3 H) 3.35 (s, 3 H) 4.40 (q, J = 7.1 Hz, 2 H) 4.69 (s, 2 H) 7.61 (s, 2 H) 7.92 (d, J = 8.7 Hz, 2 H) 8.05 (d, J = 8.7 Hz, 2 H) | | 7.07 | 5000 | |
| a- 4g | 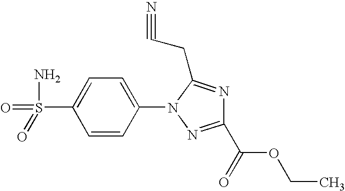 | ethyl 1-[4- (aminosulfonyl)phenyl]- 5-(cyanomethyl)-1H- 1,2,4-triazole-3- carboxylate | 336 | ¹H NMR (300 MHz, DMSO- d₆) 1.41 (t, J = 7.2 Hz, 3 H) 4.47 (q, J = 7.2 Hz, 2 H) 4.67 (s, 2 H) 7.69 (s, 2 H) 7.94 (d, J = 8.7 Hz, 2 H) 8.10 (d, J = 7.6 Hz,2 H) | | 3.24 | 1000 | |
| a- 4h | 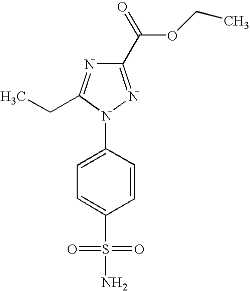 | ethyl 1-[4- (aminosulfonyl)phenyl]- 5-ethyl-1H-1,2,4- triazole-3-carboxylate | 325 | ¹H NMR (300 MHz, DMSO- d₆) 1.26 (t, J = 7.5 Hz, 3 H) 1.34 (t, J = 7.1 Hz, 3 H) 2.89 (q,J = 7.5 Hz, 2 H) 4.38 (q, J = 7.1 Hz, 2 H) 7.60 (s, 2 H) 7.67 (d, J = 8.7 Hz,2 H) 8.04 (d, J = 8.7 Hz, 2 H) | | 3.81 | 1000 | |
| a- 4i | 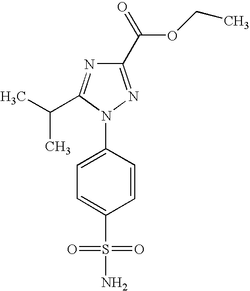 | ethyl 1-[4- (aminosulfonyl)phenyl]- 5-isopropyl-1H-1,2,4- triazole-3-carboxylate | 339 | ¹H NMR (300 MHz, DMSO- d₆) 1.29 (d, J = 6.8 Hz, 6 H) 1.38 (t,J = 7.1 Hz, 3 H) 3.22 (Septuplet, J = 6.8 Hz, 1 H) 4.42 (q, J = 7.1 Hz, 2 H) 7.66 (s, 2 H) 7.90 (d, J = 8.7 Hz, 2 H) 8.10 (d, J = 8.7 Hz, 2 H) | | 2.66 | 3400 | 0.649 |
| a-4j | 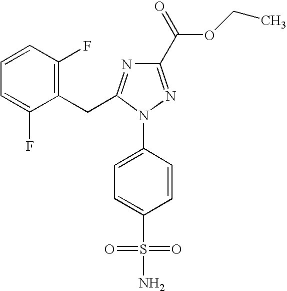 | ethyl 1-[4- (aminosulfonyl)phenyl]- 5-(2,6- difluorobenzyl)-1H- 1,2,4-triazole-3- carboxylate | 423 | ¹H NMR (300 MHz, DMSO- d₆) 1.30 (t, J = 7.2 Hz, 3 H) 4.34 (s,2 H) 4.35 (q,J = 7.2 Hz, 2 H) 7.10 (t,J = 7.9 Hz, 2 H) 7.34-7.48 (m, 1 H) 7.63 (s, 2 H) 7.94 (d, J = 8.6 Hz, 2 H) 8.05 (d, J = 8.6 Hz,2 H) | | 2.24 | 3870 | 0.251 |
| a- 4k | 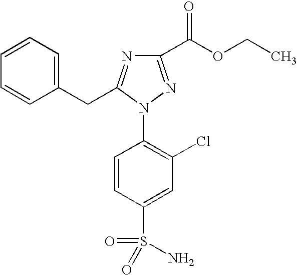 | ethyl 1-[4- (aminosulfonyl)-2- chlorophenyl]-5- benzyl-1H-1,2,4- triazole-3-carboxylate | 421 | ¹H NMR (400 MHz, CDCl₃) 1.38 (t, J = 7.1 Hz, 3 H) 4.07 (s, 2 H) 4.45 (q, J = 7.1 Hz, 2 H) 5.04 (s,2 H) 6.82-6.90 (m, 2 H) 7.05-7.14 (m,4 H) 7.70 (dd, J = 8.3, 2.0 Hz, 1 H) 8.00 (d, J = 2.0 Hz, 1 H) | | 2.32 | 1020 | 0.048 |
| a-4l | 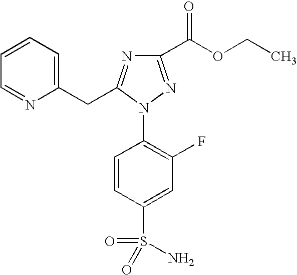 | ethyl 1-[4- (aminosulfonyl)-2- fluorophenyl]-5- (pyridin-2-ylmethyl)- 1H-1,2,4-triazole-3- carboxylate | 406 | ¹H NMR (400 MHz, CHLOROFORM-d) 1.36 (t, J = 7.1 Hz, 3 H) 4.30 (s, 2 H) 4.43 (q, J = 7.1 Hz, 2 H) 5.43 (s,2 H) 7.06 (dd,J = 7.1, 5.3 Hz,1 H) 7.15- 7.24 (m, 2 H) 7.48-7.62 (m, 2 H) 7.70 (d, J = 9.1 Hz, 2 H) | | 2.12 | 510 | 0.052 |
| A- 4m | 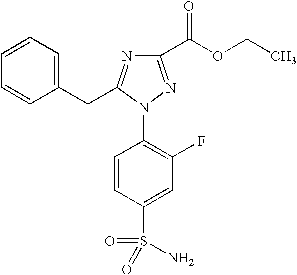 | ethyl 1-[4- (aminosulfonyl)-2- fluorophenyl]-5- benzyl-1H-1,2,4- triazole-3-carboxylate | 405 | ¹H NMR (400 MHz, CHLOROFORM-d) 1.38 (t, J = 7.1 Hz, 3 H) 4.13 (s,2 H) 4.45 (q, J = 7.1 Hz, 2 H) 4.95 (s, 2 H) 6.87- 6.93 (m, 2 H) 7.08-7.13 (m, 3 H) 7.26 (dd, J = 8.7, 1.9 Hz, 1 H) 7.65 (d, J = 8.3 Hz, 1 H) 7.70 (dd, J = 8.7, 1.9 Hz, 1 H) | | 2.34 | 2350 | |
| a- 4n | 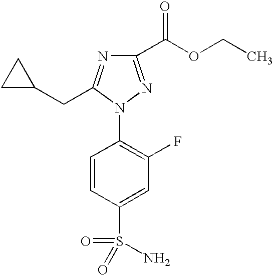 | ethyl 1-[4- (aminosulfonyl)-2- fluorophenyl]-5- (cyclopropylmethyl)- 1H-1,2,4-triazole-3- carboxylate | 369 | ¹H NMR (400 MHz, DMSO- d₆) 0.02-0.18 (m, 2 H) 0.38- 0.50 (m, 2 H) 0.92-1.06 (m, 1 H) 1.32 (t, J = 7.2 Hz, 3 H) 2.65 (d, J = 8.8 Hz, 2 H) 4.36 (q, J = 7.2 Hz, 2 H) 7.74 (s, 2 H) 7.87 (dd, J = 8.34, 1.52 Hz, 1 H) 7.93- 8.02 (m, 2 H) | | 2.00 | 642 | 0.026 |
| a- 4o | 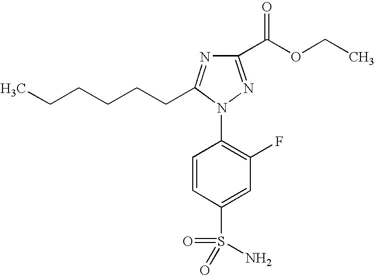 | ethyl 1-[4- (aminosulfonyl)-2- fluorophenyl]-5-hexyl- 1H-1,2,4-triazole-3- carboxylate | 399 | ¹H NMR (400 MHz, CHLOROFORM-d) 0.85 (t, J = 6.8 Hz, 3 H) 1.18-1.33 (m, 4 H) 1.45 (t, J = 7.1 Hz, 3 H) 1.57-1.70 (m, 2 H) 1.70-1.86 (m, 2 H) 2.71 (t, J = 7.1 Hz, 2 H) 4.51 (q, J = 7.1 Hz, 2 H) 5.31 (s,2 H) 7.64-7.69 (m, 1 H) 7.87-7.93 (m, 2 H) | | 2.24 | 2420 | 0.49 |
| a- 4p | 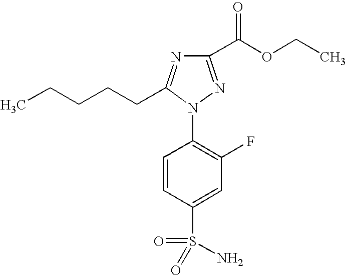 | ethyl 1-[4- (aminosulfonyl)-2- fluorophenyl]-5-pentyl- 1H-1,2,4-triazole-3- carboxylate | 385 | ¹H NMR (400 MHz, CHLOROFORM-d) 0.87 (t, J = 6.56 Hz, 3 H) 1.24-1.33 (m,4 H) 1.48 (t, J = 7.1 Hz, 3 H) 1.60-1.72 (m, 2 H) 1.72- 1.86 (m, 2 H) 2.72 (t, J = 7.8 Hz, 2 H) 4.53 (q, J = 7.1 Hz, 2 H) 5.27 (s, 2 H) 7.65-7.71 (m, 1 H) 7.89-7.96 (m, 2 H) | | 2.48 | 570 | |
| a- 4q | 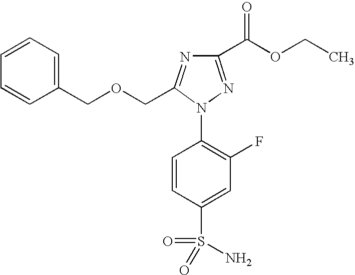 | ethyl 1-[4- (aminosulfonyl)-2- fluorophenyl]-5- [(benzyloxy)methyl]- 1H-1,2,4-triazole-3- carboxylate | 435 | ¹H NMR (400 MHz, CHLOROFORM-d) 1.47 (t, J = 7.2 Hz, 3 H) 4.45 (s, 2 H) 4.54 (q, J = 7.2 Hz, 2 H) 4.81 (s, 2 H) 5.09 (s, 2 H) 7.02-7.07 (m, 2 H) 7.27- 7.32 (m, 3 H) 7.67-7.72 (m, 1 H) 7.77-7.85 (m, 2 H) | | 2.05 | 959 | |
| a-4r | 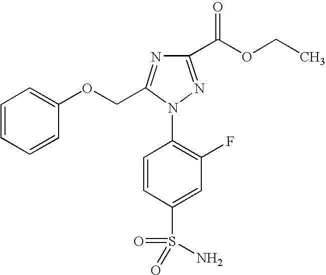 | ethyl 1-[4- (aminosulfonyl)-2- fluorophenyl]-5- (phenoxymethyl)-1H- 1,2,4-triazole-3- carboxylate | 421 | ¹H NMR (400 MHz, CHLOROFORM-d) 1.48 (t, J = 7.1 Hz, 3 H) 4.55 (q, J = 7.1 Hz, 2 H) 5.16 (s, 2 H) 5.39 (s, 2 H) 6.63 (d, J = 7.6 Hz, 2 H) 6.98 (t, J = 7.3 Hz, 1 H) 7.19- 7.25 (m, 2 H) 7.60 (dd, J = 8.5, 7.0 Hz, 1 H) 7.81-7.86 (m, 2 H) | | 2.27 | 1210 | 0.462 |
| a- 4s | 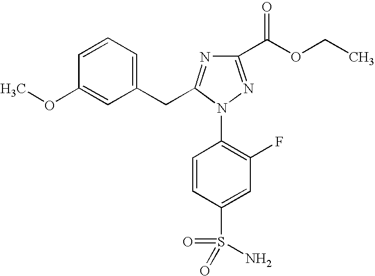 | ethyl 1-[4- (aminosulfonyl)-2- fluorophenyl]-5-(3- methoxybenzyl)-1H- 1,2,4-triazole-3- carboxylate | 435 | ¹H NMR (400 MHz, CHLOROFORM-d) 1.38 (t, J = 7.2 Hz, 3 H) 3.62 (s, 3 H) 4.09 (s,2 H) 4.44 (q, J = 7.2 Hz, 2 H) 5.01 (s, 2 H) 6.38 (s, 1 H) 6.44 (d, J = 7.6 Hz, 1 H) 6.62 (dd, J = 8.2, 2.4 Hz, 1 H) 7.00 (t, J = 7.9 Hz, 1 H) 7.24 (dd, J = 8.2, 6.9 Hz, 1 H) 7.63 (d, J = 8.6 Hz,1 H) 7.69 (dd, J = 8.6, 1.9 Hz, 1 H) | | 2.31 | 510 | 0.465 |
| a-4t | 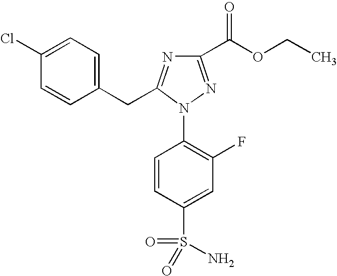 | ethyl 1-[4- (aminosulfonyl)-2- fluorophenyl]-5-(4- chlorobenzyl)-1H- 1,2,4-triazole-3- carboxylate | 439 | ¹H NMR (400 MHz, CHLOROFORM-d) 1.37 (t, J = 7.1 Hz, 3 H) 4.08 (s,2 H) 4.44 (q, J = 7.1 Hz, 2 H) 4.95 (s, 2 H) 6.87 (d, J = 8.3 Hz, 2 H) 7.09 (d, J = 8.3 Hz, 2 H) 7.30- 7.38 (m, 1 H) 7.68-7.75 (m, 2 H) | | 2.31 | 477 | 0.39 |
| a- 4u | 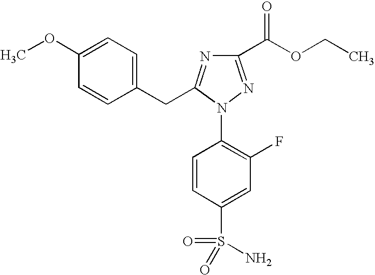 | ethyl 1-[4- (aminosulfonyl)-2- fluorophenyl]-5-(4- methoxybenzyl)-1H- 1,2,4-triazole-3- carboxylate | 435 | ¹H NMR (400 MHz, CHLOROFORM-d) d1.38 (t, J = 7.1 Hz, 3 H) 3.67 (s, 3 H) 4.07 (s,2 H) 4.45 (q, J = 7.` Hz, 2 H) 4.99 (s, 2 H) 6.62 (d, J = 8.6 Hz, 2 H) 6.80 (d, J = 8.6 Hz, 2 H) 7.23- 7.29 (m, 1 H) 7.63-7.72 (m, 2 H) | | 2.58 | 629 | |
| a- 4v | 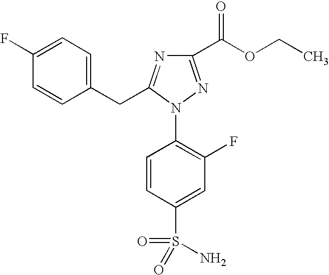 | ethyl 1-[4- (aminosulfonyl)-2- fluorophenyl]-5-(4- fluorobenzyl)-1H- 1,2,4-triazole-3- carboxylate | 423 | ¹H NMR (400 MHz, CHLOROFORM-d) 1.37 (t, J = 7.1 Hz, 3 H) 4.08 (s,2 H) 4.44 (q, J = 7.1 Hz, 2 H) 4.98 (s, 2 H) 6.77- 6.84 (m, 2 H) 6.87-6.93 (m, 2 H) 7.31 (dd, J = 8.2, 7.0 Hz, 1 H) 7.67-7.75 (m, 2 H | | 3.19 | 779 | |
| a- 4w | 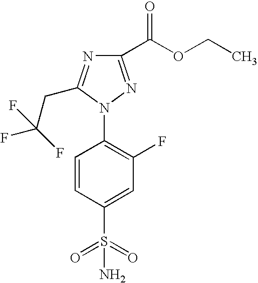 | ethyl 1-[4- (aminosulfonyl)phenyl]- 5-(2,2,2- trifluoroethyl)-1H- 1,2,4-triazole-3- carboxylate | 379 | ¹H NMR (400 MHz, DMSO- d₆) 1.33 (t, J = 7.1 Hz, 3 H) 4.23 (q, J = 10.36 Hz, 2 H) 4.38 (q, J = 7.1 Hz, 2 H) 7.62 (s, 2 H) 7.87 (d, J = 8.6 Hz, 2 H) 8.04 (d, J = 8.6 Hz, 2 H) | | 2.07 | 850 | 0.439 |
| a- 4x | 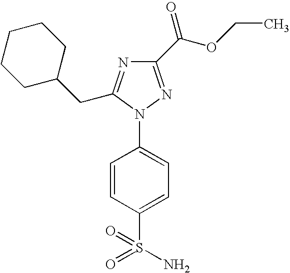 | ethyl 1-[4- (aminosulfonyl)phenyl]- 5-(cyclohexylmethyl)- 1H-1,2,4-triazole-3- carboxylate | 393 | ¹H NMR (400 MHz, DMSO- d₆) 0.61-0.91 (m, 2 H) 1.06- 1.18 (m, 3 H) 1.32 (t, J = 7.2 Hz, 2 H) 1.53- 1.62 (m, 5 H) 2.72 (d, J = 7.1 Hz, 2 H) 4.36 (q, J = 7.1 Hz, 2H) 7.60 (s, 2H) 7.82 (d, J = 8.6 Hz, 2 H) 8.03 (d, J = 8.8 Hz, 2 H) | | 2.50 | 3370 | 0.303 |
| a- 4y | 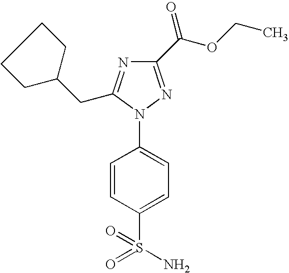 | ethyl 1-[4- (aminosulfonyl)phenyl]- 5- (cyclopentylmethyl)- 1H-1,2,4-triazole-3- carboxylate | 379 | ¹H NMR (400 MHz, DMSO- d₆) 1.04-1.15 (m, 2 H) 1.32 (t, J = 7.1 Hz, 3 H) 1.43-1.55 (m, 4 H) 1.65- 1,75 (m, 2 H) 2.86 (d, J = 7.3 Hz, 2 H) 4.36 (q, J = 1.7 Hz, 2 H) 7.59 (s, 2 H) 7.85 (d, J = 8.6 Hz, 2 H) 8.02 (d, J = 8.6 Hz, 2 H) | | 2.69 | 1230 | 0.728 |
| a- 4z | 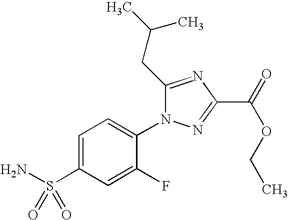 | ethyl 1-[4- (aminosulfonyl)-2- fluorophenyl]- 5-isobutyl-1H-1,2,4- triazole-3-carboxylate | 371 | ¹H NMR (300 MHz, DMSO- d6) 0.86 (d, J = 6.59 Hz, 6 H) 1.34 (t, J = 7.06 Hz,3 H) 2.00-2.12 (m, 1 H) 2.61 (d, J = 7.16 Hz, 2 H) 4.38 (q, J = 7.10 Hz, 2 H) 7.75 (br. s., 2 H) 7.87-8.04 (m, 3 H) | | 2.98 | 1900 | 0.95 |
| a-5 | 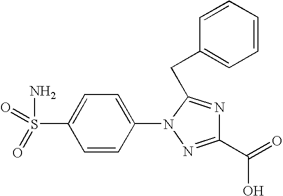 | 1-[4- (aminosulfonyl)phenyl]- 5-benzyl-1H-1,2,4- triazole-3-carboxylic acid | 359 | ¹H NMR (300 MHz, DMSO- d₆) 4.31 (s, 2 H) 7.11-7.19 (m, 2 H) 7.21- 7.35 (m, 3 H) 7.60 (s, 2 H) 7.82 (d, J = 8.7 Hz, 2 H) 8.01 (d, J = 8.7 Hz, 2 H) 13.30- 13.54 (bs, 1 H) | | 2.54 | 427 | 4.65 |
| a- 5a | 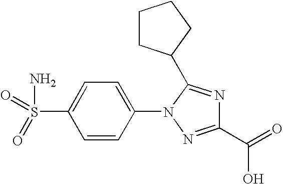 | 1-[4- (aminosulfonyl)phenyl]- 5-cyclopentyl-1H- 1,2,4-triazole-3- carboxylic acid | 337 | ¹H NMR (300 MHz, DMSO- d₆) 1.49-1.65 (m, 2 H) 1.68- 1.88 (m, 4 H) 1.89-2.08 (m, 2 H) 3.17-3.32 (m,1 H) 7.61 (s, 1 H) 7.83 (d, J = 8.6 Hz, 2 H) 8.05 (d, J = 8.6 Hz, 2 H) | | 6.46 | 1000 | |
| a- 5b | 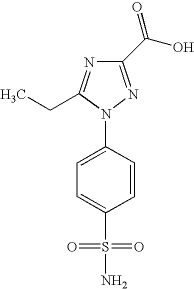 | 1-[4- (aminosulfonyl)phenyl]- 5-ethyl-1H-1,2,4- triazole-3-carboxylic acid | 297 | ¹H NMR (300 MHz, DMSO- d₆) 1.26 (t, J = 7.4 Hz, 3 H) 2.88 (q, J = 7.4 Hz,2 H) 7.60 (s, 2 H) 7.86 (d, J = 8.7 Hz, 2 H) 8.04 (d, J = 8.7 Hz, 2 H) 13.50 (s, 1 H) | | 17.10 | 3460 | |
| a- 5c | 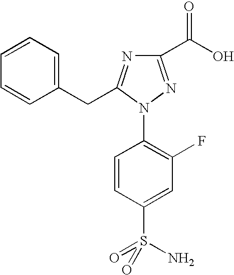 | 1-[4-(aminosulfonyl)-2- fluorophenyl]-5- benzyl-1H-1,2,4- triazole-3-carboxylic acid | 377 | ¹H NMR (400 MHz, MeOD) 4.11 (s, 2 H) 6.82-6.92 (m, 2 H) 7.04-7.14 (m, 3 H) 7.52 (t, J = 7.6 Hz, 1 H) 7.66-7.76 (m, 2 H) | | 2.25 | 543 | |
| B-1 | 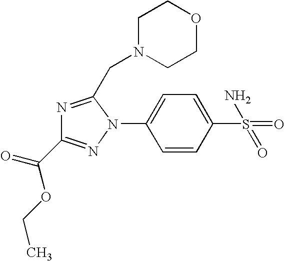 | ethyl 1-[4- (aminosulfonyl)phenyl]- 5-(morpholin-4- ylmethyl)-1H-1,2,4- triazole-3-carboxylate | 396 | ¹H NMR (300 MHz, DMSO- d₆) 1.35 (t, J = 7.1 Hz, 3 H) 4.42 (q, J = 7.1 Hz, 2 H) 7.66 (s, 2 H) 7.96 (d, J = 8.5 Hz, 5 H) 8.07 (d, J = 8.5 Hz, 5 H) | | 7.09 | 5000 | |
| B- 1a | 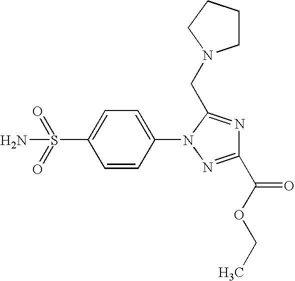 | ethyl 1-[4- (aminosulfonyl)phenyl]- 5-(pyrrolidin-1- ylmethyl)-1H-1,2,4- triazole-3-carboxylate | 380 | ¹H NMR (300 MHz, DMSO- d₆) 1.36 (t, J = 7.1 Hz, 3 H) 1.86-2.07 (m, 4 H) 3.34-3.61 (m, 4 H) 4.43 (q, J = 7.1 Hz, 2 H) 4.85 (s, 2 H) 7.66 (s, 2 H) 7.91 (d,J = 8.7 Hz, 2 H) 8.07 (d, J = 8.7 Hz, 2 H) | | 3.39 | 1000 | |
| C-1 | 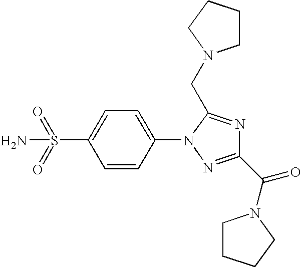 | 4-[3-(pyrroliddin-1- ylcarbonyl)-5- (pyrrolidin-1-ylmethyl)- 1H-1,2,4-triazol-1- yl]benzenesulfonamide | 405 | ¹H NMR (300 MHz, DMSO- d₆) 1.36 (t, J = 7.1 Hz, 3 H) 1.83-2.10 (m, 4 H) 3.24-3.69 (m, 12 H) 4.43 (q, J = 7.1 Hz, 2 H) 4.85 (s,2 H) 7.66 (s, 2 H) 7.91 (d, J = 8.5 Hz, 2 H) 8.08 (d, J = 8.5 Hz, 2 H) | | 12.10 | 1000 | |
| C- 1a | 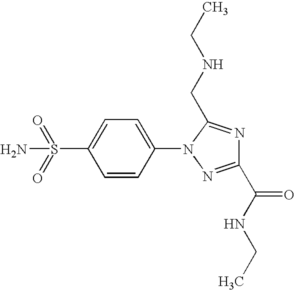 | 1-[4- (aminosulfonyl)phenyl]- N-ethyl-5- [(ethylamino)methyl]- 1H-1,2,4-triazole-3- carboxamide | 353 | ¹H NMR (300 MHz, DMSO- d₆) d 1.15 (t, J = 7.2 Hz, 3 H) 1.25 (t,J = 7.2 Hz, 3 H) 3.06- 3.24 (m, 2 H) 3.26-3.42 (m, 2 H) 4.62 (s, 2 H) 7.65 (s, 2 H) 7.91 (d, J = 8.7 Hz, 2 H) 8.08 (d, J = 8.7 Hz, 2 H) 8.71 (t, J = 5.84 Hz,1 H) 9.43 (s, 1 H) | | 78.90 | >5000 | |
| D-1 | 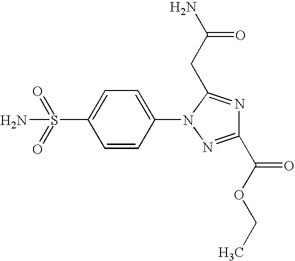 | ethyl 5-(2-amino-2- oxoethyl)-1-[4- (aminosulfonyl)phenyl]- 1H-1,2,4-triazole-3- carboxylate | 354 | ¹H NMR (300 MHz, DMSO- d₆) 1.34 (t, J = 7.1 Hz, 3 H) 3.88 (s, 2 H) 4.39 (q, J = 7.1 Hz, 2 H) 7.24 (s, 1 H) 7.61 (s, 2 H) 7.74 (s, 1 H) 7.90 (d, J = 8.5 Hz, 2 H) 8.03 (d, J = 8.5 hz, 2 H) | | 7.19 | 1000 | |
| E-1 | 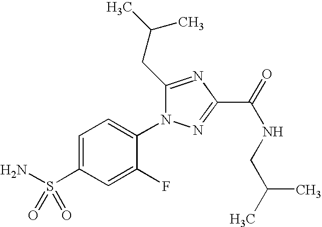 | 1-[4-(aminosulfonyl)-2- fluorophenyl]- N,5-diisobutyl-1H- 1,2,4-triazole-3- carboxamide | 398 | ¹H NMR (300 MHz, DMSO- d6) 0.88 (t, J = 6.59 Hz,2 H) 1.80-1.94 (m, 1 H) 1.99- 2.13 (m, 1 H) 2.60 (d, J = 6.97 Hz, 2 H) 3.10 (t, J = 6.50 Hz, 2 H) 7.76 (br. s., 2 H) 7.87-8.03 (m, 3 H) 8.57 (t, J = 8.03 Hz, 1 H) | | 2.34 | 2180 | 0.31 |
| E- 1a | 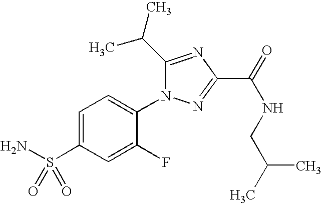 | 1-[4-(aminosulfonyl)-2- fluorophenyl]- N-isobutyl-5- isopropyl-1H-1,2,4- triazole-3- carboxamide | 384 | ¹H NMR (300 MHz, DMSO- d6) 0.89 (d, J = 6.59 Hz, 6 H) 1.24 (d, J = 6.78 Hz, 6 H) 1.61-1.95 (m, 1 H) 2.89- 3.00 (m,1 H) 3.09 (t, J = 6.78 Hz, 2 H) 7.76 (br. s., 2 H) 7.87-7.93 (m, 1 H) 7.96-8.07 (m,2 H) 8.53 (t, J = 5.93 Hz, 1 H) | | 2.30 | 1970 | 0.087 |
| E- 1b | 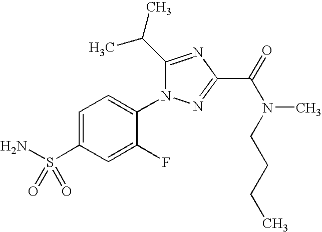 | 1-[4-(aminosulfonyl)-2- fluorophenyl]- N-butyl-5-isopropyl- N-methyl-1H-1,2,4- triazole-3- carboxamide | 398 | ¹H NMR (300 MHz, DMSO- d6) anisotropism 0.84 (t, J = 7.335 Hz, 1 H) 0.99 (t,J = 7.35 Hz, 1 H) 1.18-1.24 (m, 4 H) 1.27 (dd, J = 6.69, 1.79 Hz, 6 H) 1.33-1.42 (m, 1 H) 1.56-1.67 (m, 2 H) 2.95- 3.18 (m, 5 H) 3.48 (dt, J = 14.84, 7.44 Hz, 2 H) 7.82 (br. s., 2 H) 7.92-7.96 (m, 1 H) 8.01-8.10 (m, 2 H) | | 2.91 | 812 | 0.282 |
| F-1 | 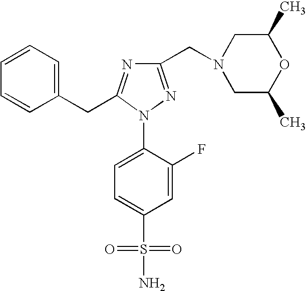 | 4-(5-benzyl-3- {[(2R,6S)-2,6- dimethylmorpholin-4- yl]methyl}-1H-1,2,4- triazol-1-yl)-3- fluorobenzenesulfonamide | 460 | ¹H NMR (300 MHz,DMSO- d₆) 1.15 (d, J = 6.2 Hz, 6 H) 2.87-2.88 (m, 2 H) 3.41-3.58 (m, 2 H) 3.86- 4.03 (m, 2 H) 4.19 (s, 2 H) 4.48 (s, 2 H) 7.06-7.13 (m, 2 H) 7.21-7.31 (m, 3 H) 7.77 (s, 2 H) 7.84- 7.96 (m,3 H) | | 2.33 | 434 | 0.187 |
| F- 1a | 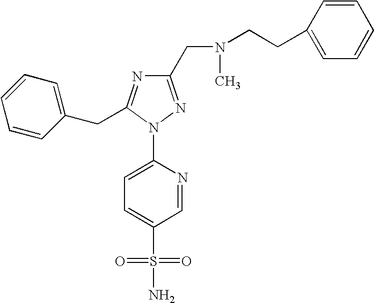 | 6-(5-benzyl-3- {[methyl(2- phenylethyl)amino] methyl}-1H-1,2,4-triazol-1- yl)pyridine-3- sulfonamide | 463 | ¹H NMR (300 MHz, DMSO- d₆) 2.95 (s, 3 H) 3.06-3.16 (m, 2 H) 4.61 (s,2 H) 4.74 (d, J = 2.1 Hz, 2 H) 7.20-7.41 (m, 10 H) 7.81 (s, 2 H) 8.08 (dd, J = 8.7, 0.7 Hz, 1 H) 8.48 (dd, J = 8.7, 2.4 Hz, 1 H) 8.98 (dd, J = 2.4, 0.7 Hz, 1 H) 10.81 (s,1 H) | | 3.23 | 157 | |
| F- 1aa | 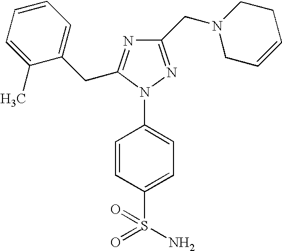 | 4-[3-(3,6- dihydropyrrolidin-1(2H)- ylmethyl)-5-(2- methylbenzyl)-1H- 1,2,4-triazol-1- yl]benzenesulfonamide | 424 | ¹H NMR (400 MHz, DMSO- d₆) 2.04-2.12 (m, 5 H) 2.63 (bs, 2 H) 3.01 (bs, 2 H) 3.58- 3.68 (m, 2 H) 4.22 (s, 2 H) 5.61-5.71 (m, 2 H) 6.91 (d, J = 7.3 Hz, 1H) 7.04-7.15 (m, 3 H) 7.54 (s, 2 H) 7.77 (d, J = 8.6 Hz, 2 H) 7.96 (d, J = 8.6 Hz, 2 H). | | 2.32 | 385 | 0.679 |
| F- 1ab | 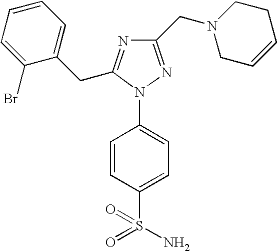 | 4-[5-(2-bromobenzyl)- 3-(3,6-dihydropyridin- 1(2H)-ylmethyl)-1H- 1,2,4-triazol-1- yl]benzenesulfonamide | 489 | ¹H NMR (400 MHz, DMSO- d₆) 2.07 (bs, 2 H) 2.57 (t, J = 5.7 Hz, 2 H) 2.94 (s, 2 H) 3.57 (s, 2 H) 4.31 (s, 2 H) 5.59-5.69 (m, 2 H) 7.20-7.22 (m, 1 H) 7.35 (d, J = 4.3 Hz, 2 H) 7.53-7.60 (m, 3 H) 7.81 (d, J = 8.6 Hz, 2 H) 7.99 (d, J = 8.6 Hz, 2 H). | | 2.27 | 503 | 0.321 |
| F- 1ac | 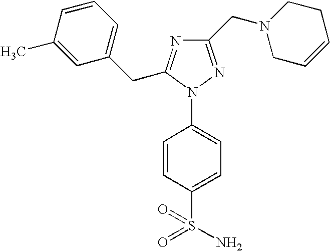 | 4-[3-(3,6- dihydropyridin-1(2H)- ylmethyl)-5-(3- methylbenzyl)-1H- 1,2,4-triazol-1- yl]benzenesulfonamide | 424 | ¹H NMR (400 MHz, DMSO- d₆) 2.07 (s, 2 H) 2.21 (s, 3 H) 2.62 (t, J = 5.7 Hz, 2 H) 3.00 (d, J = 2.5 Hz, 2 H) 3.58-3.66 (m, 2 H) 4.21 (s, 2 H) 5.61- 5.71 (m, 2 H) 6.86-6.93 (m, 2 H) 7.01 (d, J = 7.6 Hz, 1 H) 7.14 (t, J = 7.6 Hz, 1 H) 7.54 (s, 2 H) 7.72 (d, J = 8.6 Hz,2 H) 7.95 (d, J = 8.8 hz, 2 H). | | 2.35 | 166 | 0.929 |
| F- 1ad | 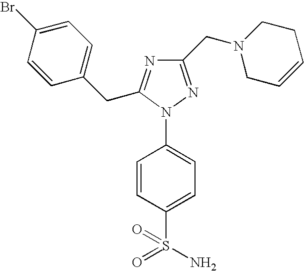 | 4-[5-(4-bromobenzyl)- 3-(3,6-dihydropyridin- 1(2H)-ylmethyl)-1H- yl]benzenesulfonamide | 489 | ¹H NMR (400 MHz, DMSO- d₆) 2.08 (s, 2 H) 2.65 (t, J = 5.1 Hz, 2 H) 3.03 (s, 2 H) 3.66 (s,2 H) 4.25 (s, 2 H) 5.61-5.73 (m, 2 H) 7.12 (d, J = 8.3 Hz,2 H) 7.47 (d, J = 8.3 Hz, 2 H) 7.55 (s, 2 H) 7.75 (d, J = 8.6 Hz, 2 H) 7.97 (d, J = 8.8 Hz, 2 H) | | 3.48 | 540 | |
| 5F- 1ae | 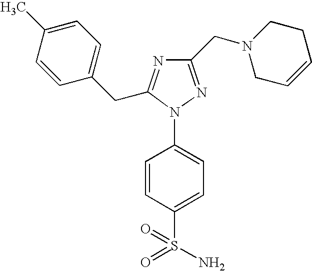 | 4-[3-(3,6- dihydropyridin-1(2H)- ylmethyl)-5-(4- methylbenzyl)-1H- 1,2,4-triazol-1- yl]benzenesulfonamide | 424 | ¹H NMR (400 MHz, DMSO- d₆) 2.09 (d, J = 2.3 Hz,2 H) 2.23 (s, 3 H) 2.66 (s, 2 H) 3.04 (s, 2 H) 3.67 (s, 2 H) 4.21 (s, 2 H) 5.62-5.73 (m, 2 H) 6.97-7.02 (m, 2 H) 7.04- 7.09 (m, 2 H) 7.54 (s, 2 H) 7.73 (d, J = 8.6 Hz, 2 H) 7.95 (d, J = 8.6 Hz, 2 H). | | 3.40 | 696 | |
| F- 1af | 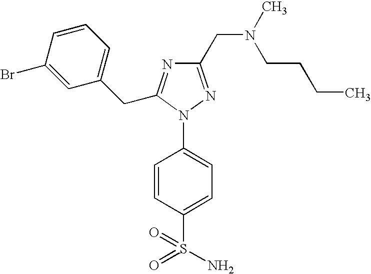 | 4-[5-(3-bromobenzyl)- 3- {[butyl(methyl)amino] methyl}-1H-1,2,4- triazol-1- yl]benzenesulfonamide | 493 | ¹H NMR (400 MHz, MeOD) 0.92 1.01 (m, 3 H) 1.36 (dt, J = 14.8, 7.4 Hz, 2 H) 1.54-1.64 (m, 2 H) 2.43 (s, 3 H) 2.56- 2.65 (m,2 H) 3.81 (s, 2 H) 4.28 (s, 2 H) 7.03-7.14 (m, 1 H) 7.17 (t, J = 7.83 Hz, 1 H) 7.30 (s, 1 H) 7.38 (d, J = 8.1 Hz,1 H) 7.63 (d, J = 8.3 Hz, 2 H) 8.06(t, J = 8.8 Hz, 2 H). | | 2.42 | 127 | 1.32 |
| F- 1ag | 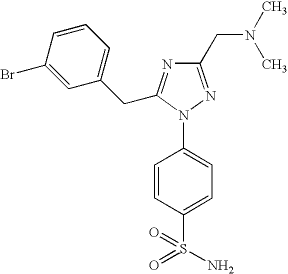 | 4-[5-(3-bromobenzyl)- 3- [(dimethylamino)methyl]- 1H-1,2,4-triazol-1- yl]benzenesulfonamide | 451 | ¹H NMR (400 MHz, DMSO- d₆) 2.21 (s, 6 H) 3.47 (s, 2 H) 4.26 (s,2 H) 7.15-7.17 (m, 1 H) 7.24 (t, J = 7.7 Hz, 1 H) 7.39-7.44 (m, 2 H) 7.54 (s, 2 H) 7.75 (d, J = 8.6 Hz, 2 H) 7.95-8.01 (m, 2 H). | | 3.06 | 192 | 1.4 |
| F- 1ah | 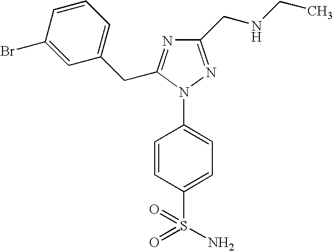 | 4-[5-(3-bromobenzyl)- 3- [(ethylamino)methyl]- 1H-1,2,4-triazol-1- yl)benzenesulfonamide | 451 | ¹H NMR (400 MHz, DMSO- d₆) 1.01 (t, J = 7.1 Hz, 3 H) 2.59 (q, J = 7.1 Hz, 2 ) 3.71 (s, 2 H) 4.26 (s, 2 H) 7.15-7.17 (m, 1 H) 7.25 (t, J = 7.8 Hz, 1 H) 7.39-7.46 (m, 2 H) 7.54 (s, 2 H) 7.75 (d, J = 8.6 Hz, 2 H) 7.97 (d, J = 8.6 Hz, 2 H). | | 4.36 | 196 | |
| F- 1ai | 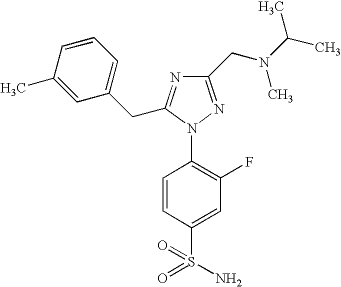 | 3-fluoro-4-[3- {[isopropyl(methyl)amino] methyl}-5-(3- methylbenzyl)-1H- 1,2,4-triazol-1- yl]benzenesulfonamide | 432 | ¹H NMR (400 MHz, MeOD) 1.17 (d,J = 6.8 Hz, 6 H) 2.18 (s, 3 H) 2.45 (s, 3 H) 3.89 (s, 2 H) 4.09 (s, 2 H) 6.73 (s, 2 H) 6.97-7.02 (m, 1 H) 7.02-7.06 (m, 1 H) 7.52- 7.56 (m, 1 H) 7.75-7.80 (m, 2 H) | | | 86 | 0.12 |
| F- 1aj | 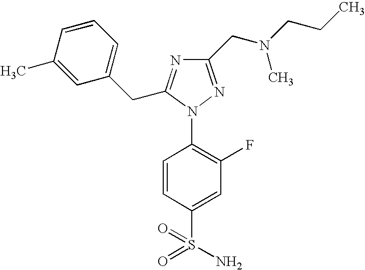 | 3-fluoro-4-[5-(3- methylbenzyl)-3- {[methyl(propyl)amino] methyl}-1H-1,2,4- triazol-1- yl]benzenesulfonamide | 432 | ¹H NMR (400 MHz, MeOD) 0.91 (t, J = 6.6 Hz, 3 H) 1.54- 1.64 (m, 2 H) 2.18 (s, 3 H) 2.18 (s, 3 H) 2.41 (s, 3 H) 2.63 (t,J = 6.6 Hz,2 H) 3.80 (s,2 H) 4.09 (s, 2 H) 6.73 (s, 2 H) 6.97-7.02 (m, 1 H) 7.02- 7.06 (m,1 H) 7.52-7.56 (m, 1 H) 7.75-7.80 (m, 2 H) | | | 87 | 0.068 |
| F- 1ak | 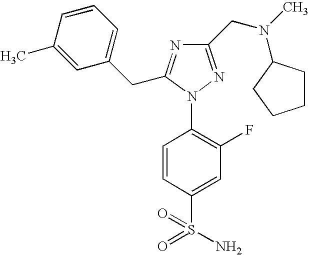 | 4-[3- {[cyclopentyl(methyl) amino]methyl}-5-(3- methylbenzyl-1H- 1,2,4-triazol-1-yl]-3- fluorobenzenesulfonamide | 458 | ¹H NMR (400 MHz, MeOD) 1.54-1.65 (m, 4 H) 1.70-1.80 (m, 2 H) 1.99- 2.09 (m, 2 H) 2.19 (s, 3 H) 2.52 (s, 3 H) 3.03-3.08 (m, 1 H) 3.98 (s,2 H) 4.11 (s, 2 H) 6.74 (s,2 H) 6.97-7.02 (m, 1 H) 7.02-7.06 (m, 1 H) 7.52- 7.56 (m, 1 H) 7.76-7.81 (m, 2 H). | | | 96 | 0.047 |
| F- 1al | 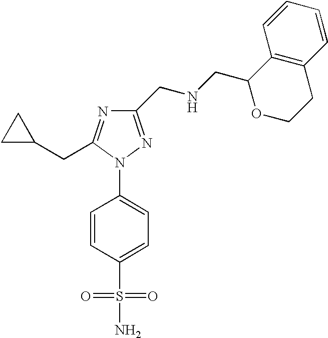 | 4-[5- (cyclopropylmethyl)-3- {[(3,4-dihydro-1H- isochromen-1- ylmethyl)amino]methyl}- 1H-1,2,4-triazol-1- yl]benzenesulfonamide | 454 | ¹H NMR (400 MHz, CHLOROFORM- D) 0.14- 0.19 (m, 2 H), 0.53-0.57 (m, 2 H), 1.06- 1.13 (m, 1 H), 1.64 (bs, 1 H) 2.71-2.73 (m, 1 H) 2.76 (m, 2 H), 2.92-3.08 (m, 2 H), 3.10- 3.20 (m, 2 H), 3.77-3.84 (m, 1 H), 3.97- 4.07 (m, 2 H), 4.12-4.19 (m, 1 H), 4.74 (m, 1 H), 4.96 (m,1 H), 7.07-7.15 (m, 3 H), 7.25- 7.21 (m, 3 H), 7.62 (ddd, J = 8.97, 2.27, 2.15 Hz,2 H), 8.06 (ddd, J = 8.84, 2.40, 2.15 Hz, 2 H) | | 7.45 | 2640 | |
| F- 1a m | 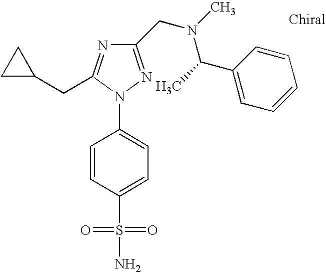 | 4-[5- (cyclopropylmethyl)-3- ({methyl[(1S)-1- phenylethyl]amino} methyl-1H-1,2,4-triazol-1- yl]benzenesulfonamide | 426 | ¹H NMR (400 MHz, CHLOROFORM- D) 0.14- 0.16 (m, 2 H), 0.50-0.56 (m, 2 H), 1.05- 1.12 (m, 1 H), 1.25 (t,J = 7.07 Hz, 3 H), 1.48 (d, J = 6.82 Hz, 2 H), 2.32 (s,2 H), 2.79 (d, J = 6.57 Hz, 1 H), 3.49 (s, 3 H), 3.61 (d, J = 14.15 Hz, 1 H), 3.70-3.80 (m, 3H), 4.98 (s, 1 H), 7.25- 7.35 (m, 3 H), 7.36-7.42 (m, 2 H), 7.62- 7.67 (m, 2 H), 8.06 (m, 2 H) | | 4.25 | 1660 | |
| F- 1an | 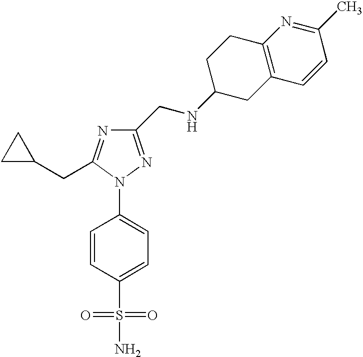 | 4-[5- (cyclopropylmethyl)-3- {[(2-methyl-5,6,7,8- tetrahydroquinolin-6- yl)amino]methyl}-1H- 1,2,4-triazol-1- yl]benzenesulfonamide | 453 | ¹H NMR (400 MHz, CHLOROFORM- D) 0.14- 0.16 (m, 2 H), 0.50-0.56 (m, 2 H), 1.03- 1.13 (m, 1 H), 1.73-1.85 (m, 1 H), 2.18 (s, 1 H0, 2.48 (s, 3 H), 2.64-2.70 (m, 1 H), 2.77 (d, J = 6.57 Hz, 2 H), 2.87- 2.96 (m, 1 H), 3.00-3.11 (m, 3 H), 4.05 (d, J = 1.26 Hz, 2 H), 5.02 (bs, 1 H), 6.90 (d, J = 7.83 Hz, 1 H), 7.28 (m, 1 H), 7.62 (ddd, J = 8.84, 2.40, 2.15 Hz, 2 H), 8.06 (ddd, J = 8.84, 2.40, 2.15 Hz, 2 H) | | 5.06 | 2260 | |
| F- 1ao | 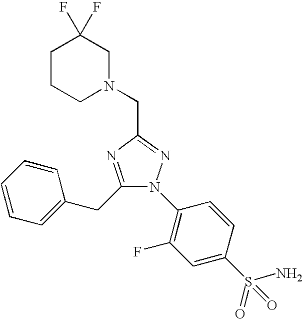 | 4-{5-benzyl-3-[(3,3- difluoropiperidin-1- yl)methyl]-1H-1,2,4- triazol-1-yl}-3- fluorobenzenesulfon- amide | 466 | ¹H NMR (400 MHz, DMSO- D6) 1.55-1.66 (m, 3 H), 1.79- 1.92 (m, 3 H), 2.59 (s, 1 H), 2.73-2.82 (m, 3 H), 3.68 (s, 2 H), 4.09 (s, 2 H), 7.02 (d, J = 7.07 Hz,2 H), 7.15-7.24 (m, 3 H), 7.67 (bs, 2 H), 7.75- 7.84 (m, 3 H) | | 2.43 | 378 | 0.081 |
| F- 1ap | 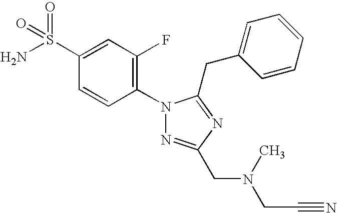 | 4-(5-benzyl-3- {[(cyanomethyl)(methyl) amino]methyl}-1H- 1,2,4-triazol-1-yl)-3- fluorobenzenesulfon- amide | 415 | ¹H NMR (400 MHz, DMSO- D6) 2.35 (s, 3 H), 3.66 (s, 3 H), 3.80 (s, 2 H), 4.09 (s, 2 H), 7.05 (d, J = 7.07 Hz, 2 H), 7.17-7.25 (m, 3 H), 7.70 (s, 2 H), 7.77- 7.85 (m, 3 H) | | 2.69 | 238 | 0.089 |
| F- 1aq | 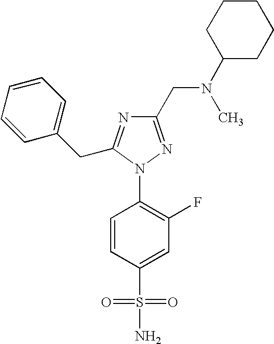 | 4-(5-benzyl-3- {[cyclohexyl(methyl) amino]methyl}-1H- 1,2,4-triazol-1-yl)-3- fluorobenzenesulfon- amide | 458 | ¹H NMR (400 MHz,DMSO- D6) 1.08-1.13 (m, 1 H), 1.14- 1.26 (m, 6 H), 1.52-1.63 (m, 2 H), 1.75 (m, 4 H), 1.93-2.01 (m, 2 H), 2.23 (bs, 1 H), 2.88 (bs, 1 H), 3.65 (bs, 1 H), 4.09 (s, 1 H), 7.03 (d, J = 6.82 Hz, 2 H), 7.16- 7.25 (m, 2 H), 7.70 (s, 2 H), 7.76-7.84 (m, 2 H), 8.52 (s, 2 H) | | 2.35 | 269 | 0.141 |
| F- 1ar | 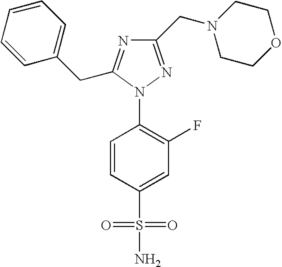 | 4-[5-benzyl-3- (morpholin-4- ylmethyl)-1H-1,2,4- triazol-1-yl]-3- fluorobenzenesulfon- amide | 432 | ¹H NMR (400 MHz, DMSO- D6) 3.31 (s, 5 H), 3.53-3.58 (m, 6 H), 4.08 (s, 2 H), 7.01- 7.05 (m,2 H), 7.16-7.24 (m, 3 H), 7.68 (s, 2 H), 7.78-7.83 (m, 3 H) | | 2.22 | 837 | 0.269 |
| F- 1as | 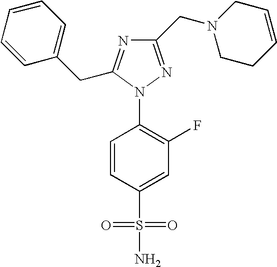 | 4-[5-benzyl-3-(3,6- dihydropyridin-1(2H)- ylmethyl)-1H-1,2,4- triazol-1-yl]-3- fluorobenzenesulfon- amide | 428 | ¹H NMR (400 MHz, DMSO- D6) 2.08 (dd, J = 3.03, 1.52 Hz, 2 H), 2.62 (t, J = 5.66 Hz, 2 H), 2.97-3.01 (m, 2 H), 3.33 (s, 3 H), 3.63 (s,2 H), 4.09 (s, 2 H), 5.67 (m, 2 H), 7.02- 7.06 (m, 2 H), 7.17-7.26 (m, 3 H), 7.69 (s,2 H), 7.77-7.84 (m, 3 H) | | 2.87 | 384 | 0.142 |
| F- 1at | 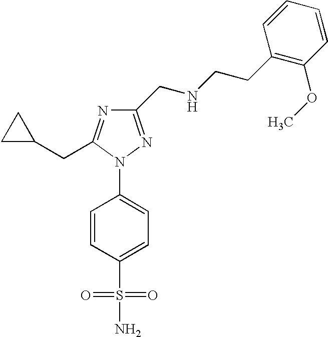 | 4-[5- (cyclopropylmethyl)-3- ({[2-(2- methoxyphenyl)ethyl] amino}methyl_1H- 1,2,4-triazol-1- yl]benzenesulfon- amide | 442 | ¹H NMR (400 MHz, CHLOROFORM- D) 0.13 0.16 (m, 2 H), 0.51-0.57 (m, 2 H), 1.04- 1.11 (m,1 H), 1.13 (bs, 2 H), 1.21 (t, J = 8.95 Hz, 1 H), 2.76 (m,3 H), 2.86- 2.91 (m, 2 H), 2.93-2.99 (m, 2 H), 3.81 (s, 3 H), 3.96 (s, 2 H), 6.83-6.90 (m, 2 H), 7.15- 7.22 (m, 2 H), 7.59 (d, J = 8.59 Hz, 2 H), 8.05 (d, J = 8.59 Hz, 2 H) | | 6.76 | 1860 | |
| F- 1au | 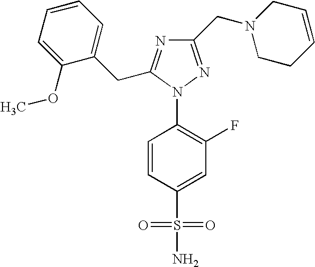 | 4-[3-(3,6- dihydropyridin-1(2H)- ylmethyl)-5-(2- methoxybenzyl)-1H- 1,2,4-triazol-1-yl]-3- fluorobenzenesulfon- amide | 458 | ¹H NMR (400 MHz, DMSO- D6) 2.09 (bs, 2 H), 2.61 (m, 2 H), 2.98 (bs, 2 H), 3.51 (s, 3 H), 3.61 (s, 2 H), 3.98 (s, 2 H), 5.62-5.70 (m, 2 H), 6.80- 6.86 (m, 2 H), 7.07-7.12 (m, 1 H), 7.15- 7.21 (m, 1 H), 7.70 (s, 2 H), 7.77-7.85 (m, 3 H) | | | 199 | 0.114 |
| F- 1av | 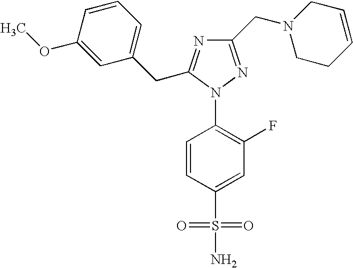 | 4-[3-(3,6- dihydropyridin-1(2H)- ylmethyl)-5-(3- methoxybenzyl)-1H- 1,2,4-triazol-1-yl]-3- fluorobenzenesulfon- amide | 458 | ¹H NMR (400 MHz, DMSO- D6) 2.07 (bs, 2 H), 2.61 (t, J = 5.56 Hz,2 H), 2.97-3.01 (m, 2 H), 3.64 (s, 2 H), 3.65 (s,3 H), 4.06 (s, 2 H), 5.62- 5.70 (m, 2 H), 6.58-6.62 (m, 2 H), 6.73- 6.78 (m, 1 H), 7.14 (t, J = 8.08 Hz, 1 H), 7.89 (s, 2 H), 7.77- 7.85 (m, 3 H) | | | 456 | 0.19 |
| F- 1a w | 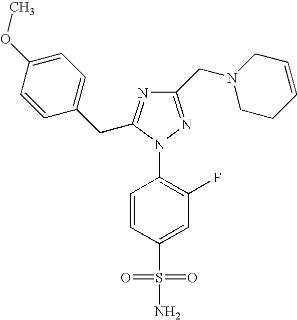 | 4-[3-(3,6- dihydropyridin-1(2H)- ylmethyl)-5-(4- methoxybenzyl)-1H- 1,2,4-triazol-1-yl]-3- fluorobenzenesulfon- amide | 458 | ¹H NMR (400 MHz, DMSO- D6) 2.07 (bs, 2 H) 2.61 (bs, 2 J = 5.68 Hz, 2 H), 2.96-3.00 (m, 2 H), 3.62 (s, 2 H), 3.69 (s, 3 H), 4.00 (s, 2 H), 5.6-1 5.71 (m, 2 H), 6.76-6.80 (m, 2 H), 6.94 (d, J = 8.59 Hz, 2 H), 7.66 (bs, 2 H), 7.77-7.84 (m, 3 H) | | | 324 | 0.035 |
| F- 1ax | 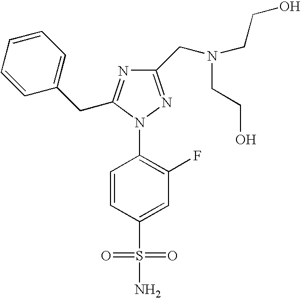 | 4-(5-benzyl-3-{[bis(2- hydroxyethyl)amino] methyl}-1H-1,2,4-triazol- 1-yl)-3- fluorobenzenesulfon- amide | 450 | ¹H NMR (400 MHz, DMSO- D6) 2.65 (m, 4 H), 3.46 (m, 4 H), 3.76 (s, 2 H), 4.10 (s, 2 H), 4.36 (m, 2 H), 7.02 (d, J = 6.82 Hz, 2 H), 7.16-7.24 (m, 3 H), 7.69 (s, 2 H), 7.75- 7.83 (m, 3 H) | | | 363 | 0.055 |
| F- 1ay | 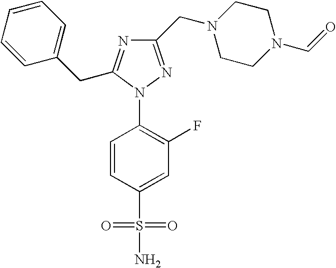 | 4-{5-benzyl-3-[(4- formylpiperazin-1- yl)methyl]-1H-1,2,4- triazol-1-yl}-3- fluorobenzenesulfon- amide | 459 | ¹H NMR (400 MHz, DMSO- D6) 2.42-2.47 (m, 2 H), 3.33- 3.40 (m, 4 H), 3.62 (s, 2 H), 4.09 (s, 2 H), 7.04 (d, J = 6.57 Hz, 2 H), 7.16- 7.25 (m, 3 H), 7.67 (bs,2 H), 7.76-7.85 (m, 3 H), 7.98 (s,1 H) | | | 646 | 0.175 |
| F- 1az | 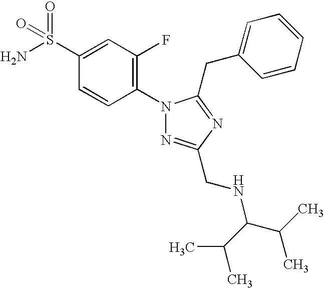 | 4-(5-benzyl-3-{[(1- isopropyl-2- methylpropyl)amino] methyl}-1H-1,2,4-triazol- 1-yl)-3- fluorobenzenesulfon- amide | 460 | ¹H NMR (400 MHz, DMSO- D6) 0.90 (dd, J = 17.81, 6.69 Hz, 12 H), 1.99- 2.09 (m, 2 H), 2.80 (m, 1 H), 4.16 (s, 2 H), 4.31 (s, 2 H), 7.05 (d, J = 6.57 Hz, 2 H), 7.22 (t, J = 7.58 Hz,3 H), 7.74 (s,2 H), 7.81-7.89 (m, 3 H), 8.74 (bs, 2 H) | | 2.70 | 166 | 0.042 |
| F- 1b | 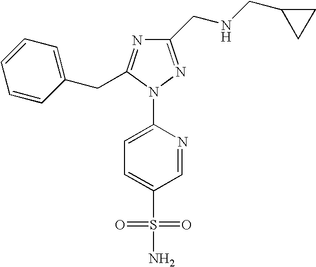 | 6-(5-benzyl-3- {[(cyclopropylmethyl) amino]methyl}-1H- 1,2,4-triazol-1- yl)pyridine-3- sulfonamide | 399 | ¹H NMR (300 MHz, DMSO- d₆) 0.34-0.44 (m, 2 H) 0.55- 0.65 (m, 2 H) 1.05-1.21 (m, 1 H) 2.97 (s, 2 H) 4.38 (s, 2 H) 4.74 (s, 2 H) 7.19-7.35 (m, 5 H) 7.81 (s, 2 H) 8.06 (d, J = 8.7, 1 H) 8.48 (dd, J = 8.7, 2.2 Hz, 1 H) 8.97 (d, J = 2.2 Hz, 1 H) 9.48 (s, 2 H) | | 4.85 | 255 | |
| F- 1ba | 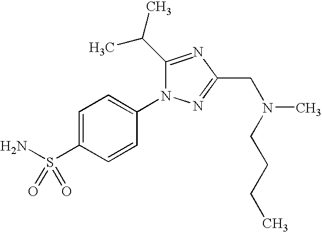 | 4-(3- {[butyl(methyl)amino] methyl}- 5-isopropyl-1H-1,2,4- triazol-1- yl)benzenesulfon- amide | 366 | ¹H NMR (300 MHz, DMSO- d6) 0.92 (t, J = 7.35 Hz, 3 H) 1.26 (d, J = 6.78 Hz, 6 H) 1.29-1.39 (m, 2 H) 1.66- 1.84 (m, 2 H) 2.84 (d, J = 4.33 Hz, 3 H) 3.00- 3.28 (m, 3 H) 44.43 (d, J = 3.58 Hz, 2 H) 7.64 (br. s., 2 H) 7.82 (d, J = 8.67 Hz, 2 H) 8.07 (d, J = 8.67 Hz, 2 H)10.91 (s, 1 H) | | 6.60 | | |
| F- 1bb | 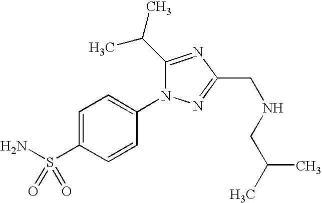 | 4-{3- [(isobutylamino)methyl]- 5-isopropyl-1H-1,2,4- triazol-1- yl}benzenesulfon- amide | 352 | ¹H NMR (300 MHz, DMSO- d6) 0.97 (d, J = 6.59 Hz, 6 H) 1.26 (d, J = 6.76 Hz, 6 H) 2.00-2.12 (m, 1 H) 2.86- 2.96 (m, 2 H) 3.16-3.28 (m, 1 H) 4.29 (t, J = 5.09 Hz, 2 H) 7.64 (br. s., 2 H) 7.80 (d, J = 8.48 Hz, 2 H) 8.07 (d, J = 8.48 Hz, 2 H) 9.43 (s, 1 H) | | 8.40 | | |
| F- 1bc | 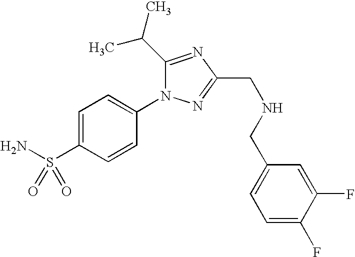 | 4-(3-{[(3,4- difluorobenzyl)- amino]methyl}-5- isopropyl-1H-1,2,4- triazol-1- yl)benzenesulfon- amide | 422 | ¹H NMR (300 MHz, DMSO- d6) 1.25 (d, J = 6.76 Hz, 6 H) 3.16-3.27 (m, 1 H) 4.26- 4.35 (m, 4 H) 7.40-7.76 (m, 5 H) 7.79 (d, J = 8.67 Hz, 2 H) 8.07 (d, J = 8.48 Hz, 2 H) 10.07 (s, 1 H) | | 8.10 | 1520 | |
| F- 1bd | 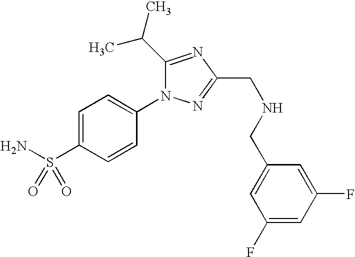 | 4-(3-{[(3,5- difluorobenzyl)amino] methyl-56 -5-isopropyl- 1H-1,2,4-triazol-1- yl)benzenesulfonamide | 422 | ¹H NMR (300 MHz, DMSO- d6) d 1.26 (d, J = 6.76 Hz, 6 H) 3.16-3.28 (m,1 H) 4.30 (s, 2 H) 4.35 (s, 2 H) 7.28-7.42 (m, 3 H) 7.63 (br. s., 2 H) 7.79 (d, J = 8.48 Hz, 2 H) 8.07 (d, J = 8.67 Hz, 2 H) 10.14 (s, 1 H) | | 5.60 | 1370 | |
| F- 1be | 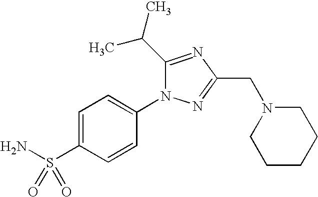 | 4-[5-isopropyl-3- (piperidin-1-ylmethyl)- 1H-1,2,4-triazol-1- yl)benzenesulfonamide | 364 | ¹H NMR (300 MHz, DMSO- d6) 1.25 (d, J = 6.78 Hz, 2 H) 1.66-1.87 (m, 6 H) 2.94- 3.10 (m, 2 H) 3.15-3.26 (m, 2 H) 3.47-3.55 (m, 2 H) 4.39- 4.42 (m, 2 H) 7.60 (br. s., 2 H) 7.83 (d, J = 8.48 Hz, 2 H) 8.07 (d, J = 8.48 Hz, 2 H) 10.73 (s, 1 H) | | 9.60 | | |
| F- 1bf | 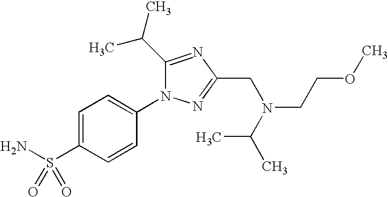 | 4-(5-isopropyl-3- {[isopropyl(2- methoxyethyl) amino]methyl}-1H- 1,2,4-triazol-1- yl)benzenesulfonamide | 396 | ¹H NMR (300 MHz, DMSO- d6) 1.25 (d, J = 6.78 Hz, 6 H) 1.34 (s, 6 H) 3.15-3.27 (m, 1 H) 3.31 (s, 3 H) 3.48 (s, 2 H) 3.63-3.80 (m, 3 H) 4.46 (s, 2 H) 7.67 (br. s., 2 H) 7.82 (d, J = 8.48 Hz, 2 H) 8.07 (d, J = 8.48 Hz, 2 H) 10.43 (br. s., 1 H) | | 7.09 | 3790 | |
| F- 1bg | 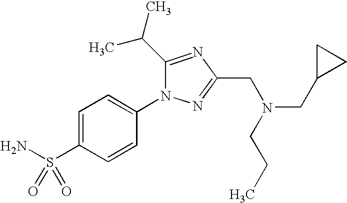 | 4-(3- {[(cyclopropylmethyl) (propyl)amino] methyl}-5-isopropyl- 1H-1,2,4-triazol-1- yl)benzenesulfonamide | 392 | ¹H NMR (300 MHz, DMSO- d6) 0.16-0.32 (m,2 H) 0.42 (d, J = 7.91 Hz, 2 H) 0.67 (t, J = 7.35 Hz, 3 H) 1.00 (d, J = 8.59 Hz, 6 H) 1.55-1.66 (m, 2 H) 2.76- 3.02 (m, 5 H) 4.25 (s,2 H) 7.40 (br. s., 2 H) 7.57 (d, J = 8.67 Hz, 2 H) 7.82 (d, J = 8.48 Hz, 2 H) 10.61 (br.s., 1 H) | | 7.97 | 3550 | |
| F- 1bh | 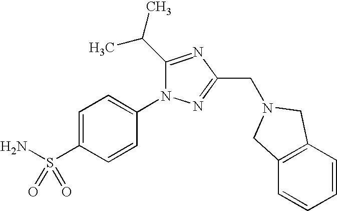 | 4-[3-(1,3-dihydro-2H- isopindol-2-ylmethyl)- 5-isopropyl-1H-1,2,4- triazol-1- yl]benzenesulfonamide | 398 | ¹H NMR (300 MHz, DMSO- d6) 1.23 (d, J = 6.78 Hz, 6 H) 3.13-3.24 (m, 1 H) 4.78 (br. s., 6 H) 7.34-7.45 (m, 4 H) 7.65 (br. s., 2 H) 7.79 (d, J = 8.67 Hz, 2 H) 8.06 (d, J = 8.48 Hz, 2 H) 12.14 (br. s., 1 H) | | 9.31 | 3790 | |
| F- 1bi | 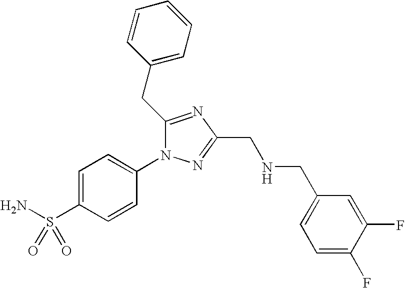 | 4-(5-benzyl-3-{[(3,4- difluorobenzyl- amino]methyl}-1H- 1,2,4-triazol-1- yl)benzenesulfonamide | 470 | ¹H NMR (300 MHz, DMSO- d6) 4.27-4.38 (m, 6 H) 7.14- 7.41 (m, 6 H) 7.48-7.67 (m, 4 H) 7.78 (d, J = 8.67 Hz,2 H) 8.02 (d, J = 8.48 Hz, 2 H) 9.74 (br. s., 1 H) | | 3.35 | 202 | |
| F- 1bj | 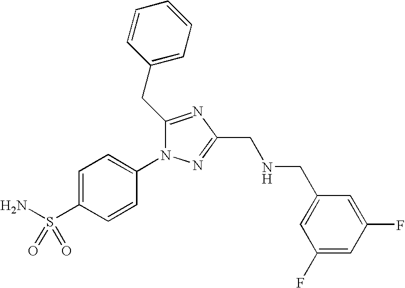 | 4-(5-benzyl-3-{[(3,5- difluorobenzyl)- amino]methyl}-1H- 1,2,4-triazol-1- yl)benzenesulfonamide | 470 | ¹H NMR (300 MHz, DMSO- d6) d 3.73 (s, 2 H) 3.81 (s, 2 H) 4.28 (s, 2 H) 7.04-7.34 (m, 8 H) 7.55 (br. s., 2 H) 7.74 (d, J = 8.67 Hz, 2 H) 7.97 (d, J = 8.67 Hz, 2 H) | | 2.77 | 199 | 0.222 |
| F- 1bk | 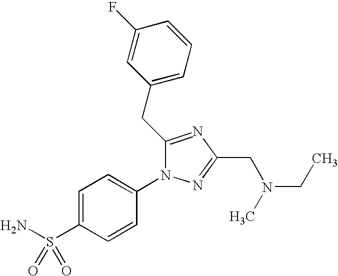 | 4-[3- {[ethyl(methyl)amino] methyl}- 5-(3-fluorobenzyl)- 1H-1,2,4-triazol-1- yl]benzenesulfonamide | 404 | ¹H NMR (300 MHz, DMSO- d6) d 1.30 (t, J = 7.16 Hz, 3 H) 2.82 (d, J = 4.33 Hz, 3 H) 3.10-3.31 (m, 2 H) 4.37 (s, 2 H) 4.48 (s, 2 H) 7.00-7.13 (m, 3 H) 7.29- 7.41 (m, 1 H) 7.63 (br. s., 2 H) 7.82 (d, J = 8.48 Hz, 2 H) 8.03 (d, J = 8.48 Hz, 2 H) 10.62 (br. s., 1 H) | | 3.50 | 1110 | |
| F- 1bl | 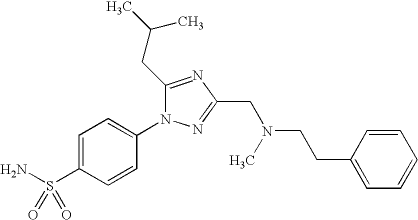 | 4-(5-isobutyl-3- {[methyl(2- phenylethyl) amino]methyl}-1H- 1,2,4-triazol-1- yl)benzenesulfonamide | 428 | 1H NMR (300 MHz, DMSO- d) d 0.89 (d, J = 6.59 Hz, 6 H) 1.99-2.17 (m, 1 H) 2.87 (d, J = 6.97 Hz, 2 H) 2.92 (d, J = 3.77 Hz, 3 H) 3.10-3.22 (m, 2 H) 3.29- 3.52 (m, 2 H) 4.55 (s, 2 H) 7.23-7.40 (m, 5 H) 7.64 (br. s., 2 H) 7.84 (d, J = 8.67 Hz, 2 H) 8.06 (d, J = 8.67 Hz, 2 H)11.24 (br. s., 1 H) | | 9.87 | 1740 | |
| F- 1b m | 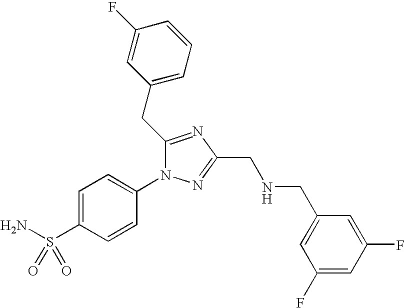 | 4-[3-{[(3,5- difluorobenzyl)amino] methyl}- 5-(3-fluorobenzyl)- 1H-1,2,4-triazol-1- yl]benzenesulfonamide | 488 | ¹H NMR (300 MHz, DMSO- d6) 4.31 (s,2 H) 4.34 (s, 2 H) 4.38 (s, 2 H) 7.02-7.14 (m, 3 H) 7.29-7.39 (m, 4 H) 7.63 (s, 2 H) 7.81 (d, J = 8.67 Hz, 2 H) 8.04 (d, J = 8.67 Hz, 2 H) 10.13 (br. s., 1 H) | | 2.72 | 357 | 0.86 |
| F- 1bn | 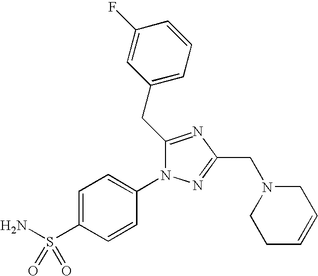 | 4-[3-(3,6- dihydropyridin-1(2H)- ylmethyl)- 5-(3-fluorobenzyl)- 1H-1,2,4-triazol-1- yl]benzenesulfonamide | 428 | ¹H NMR (300 MHz, DMSO- d6) 2.25-2.48 (m, 4 H) 3.83 (s, 2 H) 4.37 (s, 2 H) 4.54 (s, 2 H) 5.73 (d, J = 10.55 Hz, 1 H) 5.93 (d, J = 10.17 Hz,1 H) 7.01-7.13 (m, 3 H) 7.30- 7.40 (m, 1 H) 7.62 (br. s., 2 H) 7.83 (d, J = 8.48 Hz, 2 H) 8.03 (d, J = 8.67 Hz, 2 H) 10.85 (br. s., 1H) | | 3.04 | 749 | 0.826 |
| F- 1bo | 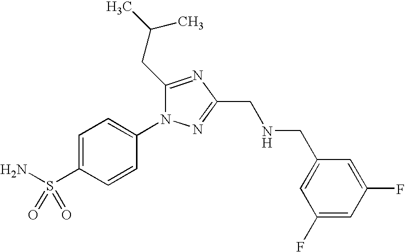 | 4-(3-{[(3,5- difluorobenzyl)amino] methyl}- 5-isobutyl-1H-1,2,4- triazol-1- yl)benzenesulfonamide | 436 | ¹H NMR (300 MHz, DMSO- d6) 0.89 (d, J = 6.59 Hz, 6 H) 1.24 (d, J = 6.78 Hz, 6 H) 1.81-1.95 (m, 1 H) 2.89- 3.00 (m, 1 H) 3.09 (t, J = 6.78 Hz, 2 H) 7.76 (br. s., 2 H) 7.87-7.93 (m, 1 H) 7.98-8.07 (m, 2 H) 8.53 (t, J = 5.93 Hz, 1 H) | | 12.70 | 3050 | |
| F- 1bp | 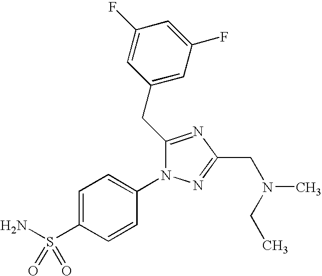 | 4-[5-(3,5- difluorobenzyl)-3- {[ethyl(methyl)amino] methyl}-1H-1,2,4- triazol-1- yl)benzenesulfonamide | 422 | ¹H NMR (300 MHz, DMSO- d6) 1.30 (t, J = 7.25 Hz, 3 H) 2.82 (d, J = 4.52 Hz, 3 H) 3.12-3.28 (m, 2 H) 4.39 (s, 2 H) 4.46 (s, 2 H) 7.02 (d, J = 6.59 Hz, 2 H) 7.10-7.27 (m, 1 H) 7.64 (br. s., 2 H) 7.84 (d, J = 8.67 Hz, 2 H) 8.05 (d, J = 8.48 Hz, 2 H) 10.87 (br. s., 1H) | | 4.18 | 1720 | |
| F- 1bq | 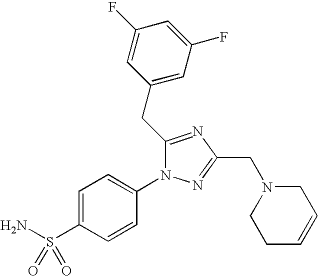 | 4-[5-(3,5- difluorobenzyl)-3-(3,6- dihydropyridin-1(2H)- ylmethyl)-1H-1,2,4- triazol-1- yl]benzenesulfonamide | 446 | ¹H NMR (300 MHz, DMSO- d6) 2.25-2.49 (m, 2 H) 3.16- 3.29 (m, 1 H) 3.60-3.65 (m, 1 H) 3.83 (s, 2 H) 4.39 (s, 2 H) 4.53 (s, 2 H) 5.73 (d, J = 10.36 Hz,1 H) 5.93 (d, J = 1.17 Hz, 1 H) 7.02 (d, J = 6.59 Hz, 2 H) 7.09-7.19 (m, 1 H) 7.64 (s, 2 H) 7.84 (d, J = 8.67 Hz,2 H)8.05 (d, J = 8.48 Hz, 2 H) 10.94 (br. s., 1 H) | | 2.37 | 922 | 1.65 |
| F- 1br | 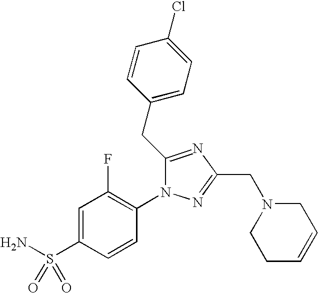 | 4-[5-(4-chlorobenzyl)- 3-(3,6-dihydropyridin- 1(2H)-ylmethyl)- 1H-1,2,4-triazol-1-yl]- 3- fluorobenzenesulfonamide | 482 | ¹H NMR (300 MHz, DMSO- d6) 2.21-2.47 (m, 1 H) 3.09- 3.27 (m, 1 H) 3.48-3.88 (m, 4 H) 4.08 (s, 1 H) 4.17 (s, 1 H) 4.46 (s, 1 H) 4.53 (s, 1 H) 5.72 (d, J = 8.29 Hz, 1 H) 5.92 (d, J = 10.1 Hz, 1 H) 7.11-7.19 (m,2 H) 7.29- 7.37 (m, 2 H) 7.74 (s, 2 H) 7.77-7.94 (m, 3 H) 10.83(br. s., 1 H) | | 2.20 | 340 | 0.057 |
| F- 1bs | 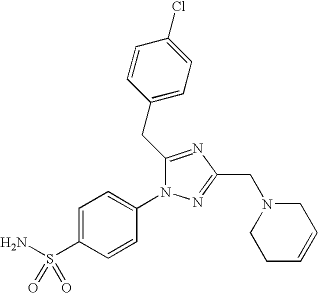 | 4-[5-(4-chlorobenzyl)- 3-(3,6-dihydropyridin- 1(2H)-ylmethyl)- 1H-1,2,4-triazol-1- yl]benzenesulfonamide | 444 | ¹H NMR (300 MHz, DMSO- d6) 2.25-2.45 (m, 1 H) 3.16- 3.19 (m, 1 H) 3.35-3.64 (m, 2 H) 3.80 (s, 2 H) 4.32 (s, 2 H) 4.50 (s, 2 H) 5.71 (d, J = 9.42 Hz, 1 H) 5.91 (d, J = 9.04 Hz, 1 H) 7.19-7.25 (m, 2 H) 7.32- 7.38 (m, 2 H) 7.61 (s, 2 H) 7.77-7.84 (m, 2 H) 7.98-8.04 (m, 2 H) 10.94 (br. s.,1 H) | | 2.40 | 687 | |
| F- 1bt | 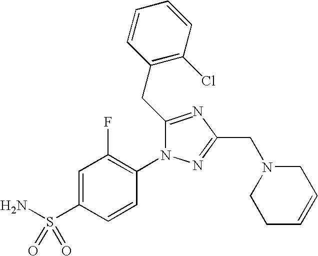 | 4-[5-(2-chlorobenzyl)- 3-(3,6-dihydropyridin- 1(2H)-ylmethyl)- 1H-1,2,4-triazol-1-yl]- 3- fluorobenzenesulfonamide | 462 | ¹H NMR (300 MHz, DMSO- d6) 2.22-2.46 (m, 2 H) 3.07- 3.26 (m, 1 H) 3.52-3.59 (m, 1 H) 3.76 (s, 2 H) 4.26 (s, 2 H) 4.52 (s, 2 H) 5.71 (d, J = 10.38 Hz, 1 H) 5.90 (d, J = 9.42 Hz, 1 H) 7.25-7.42 (m, 4 H) 7.78 (s, 2 H) 7.84- 8.00 (m, 3 H) 10.80 (br. s., 1 H) | | 2.30 | 307 | |
| F- 1bu | 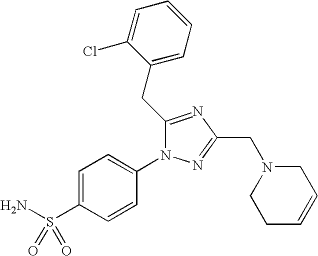 | 4-[5-(2-chlorobenzyl)- 3-(3,6-dihydropyridin- 1(2H)-ylmethyl)- 1H-1,2,4-triazol-1- yl]benzenesulfonamide | 444 | ¹H NMR (300 MHz, DMSO- d) 2.23-2.46 (m, 2 H) 3.09- 3.21 (m, 1 H) 3.53-3.59 (m, 1 H) 3.76 (s, 2 H) 4.38 (s, 2 H) 4.49 (s, 2 H) 5.70 (d, J = 10.55 Hz, 1 H) 5.89 (d, J = 9.04 Hz,1 H) 7.27-7.33 (m, 2 H) 7.36- 7.45 (m, 2 H) 7.62 (s, 2 H) 7.86 (d, J = 7.91 Hz, 2 H) 8.03 (d, J = 8.29Hz, 2 H) 10.86 (br. s., 1 H) | | 2.40 | 527 | |
| F- 1bv | 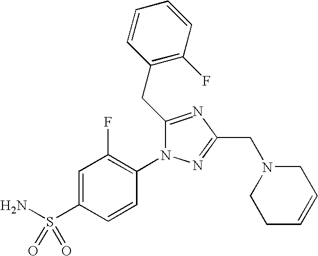 | 4-[3-(3,5- dihydropyridin-1(2H)- ylmethyl)-5-(2- fluorobenzyl)- 1H-1,2,4-triazol-1-yl]- 3- fluorobenzenesulfonamide | 446 | ¹H NMR (300 MHz, DMSO- d) 2.26-2.46 (m, 2 H) 3.11- 3.26 (m, 1 H) 3.53-3.83 (m, 1 H) 3.79 (s, 2 H) 4.21 (s, 2 H) 4.54 (s, 2 H) 5.73 (d, J = 10.36 Hz, 1 H) 5.92 (d, J = 10.36 Hz, 1 H) 7.08-7.18 (m, 2 H) 7.21- 7.35 (m, 2 H) 7.81 (s, 2 H) 7.85-8.01 (m, 3 H) 10.91 (br. s., 1H) | | 2.14 | 489 | 0.051 |
| F- 1b w | 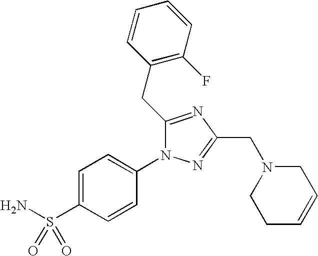 | 4-[3-(3,6- dihydropyridin-1(2H)- ylmethyl)-5-(2- fluorobenzyl)- 1H-1,2,4-triazol-1- yl]benzenesulfonamide | 428 | ¹H NMR (400 MHz, DMSO- d6) 2.29-2.44 (m, 2 H) 3.17- 3.21 (m, 4 H) 3.55-3.60 (m, 1 H) 3.78 (s, 2 H) 4.34 (s, 2 H) 4.50 (s, 2 H) 5.71 (d, J = 10.36 Hz, 1 H) 5.90 (d, J = 10.36 Hz, 1 H) 7.09-7.19 (m, 2 H) 7.27- 7.33 (m, 2 H) 7.62 (s, 2 H) 7.86 (d, J = 8.59 Hz, 2 H) 8.03 (d, J= 8.59 Hz, 2 H) 10.83 (br. s., 1 H) | | 2.64 | 851 | 0.175 |
| F- 1bx | 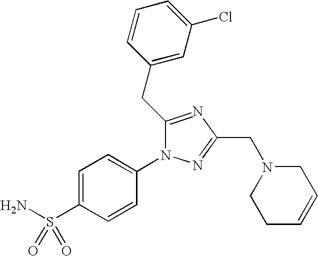 | 4-[5-(3-chlorobenzyl)- 3-(3,6-dihydropyridin- 1(2H)- ylmethyl)-1H-1,2,4- triazol-1- yl]benzenesulfonamide | 444 | ¹H NMR (300 MHz, DMSO- d6) 2.22-2.49 (m, 2 H) 3.14- 3.25 (m, 1 H) 3.60-3.68 (m, 1 H) 3.82 (s, 2 H) 4.36 (s, 2 H) 4.52 (s,2 H) 5.73 (d, J = 10.36 Hz, 1 H) 5.92 (d, J = 10.17 Hz, 1 H) 7.16-7.21 (m, 1 H) 7.30- 7.36 (m, 3 H) 7.65 (s, 2 H) 7.84 (d, J = 8.67 Hz, 2 H) 8.04 (d, J= 8.67 Hz, 2 H) 11.04 (br. s., 1 H) | | 2.74 | 200 | 1.14 |
| F- 1by | 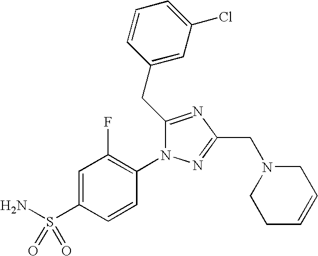 | 4-[5-(3-chlorobenzyl)- 3-(3,6-dihydropyridin- 1(2H)- ylmethyl)-1H-1,2,4- triazol-1-yl]-3- fluorobenzenesulfonamide | 462 | ¹H NMR (300 MHz, DMSO- d6) 2.24-2.49 (m, 2 H) 3.14- 3.27 (m, 1 H) 3.52-3.66 (m, 1 H) 3.82 (s, 2 H) 4.22 (s, 2 H) 4.55 (s, 2 H) 5.73 (d, J = 10.55 Hz, 1 H) 5.93 (d, J = 10.36 Hz, 1 H) 7.10-7.17 (m, 1 H) 7.23- 7.36 (m, 3 H) 7.81 (s, 2 H) 7.86-8.00 (m, 3 H) 10.93 (br. s., 1H) | | 2.58 | 307 | 0.271 |
| F- 1bz | 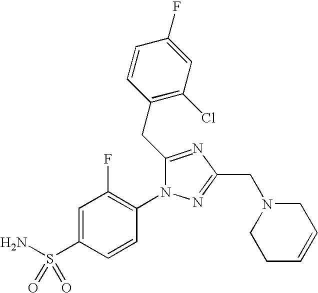 | 4-[5-(2-chloro-4- fluorobenzyl)-3-(3,6- dihydropyridin-1(2H)- ylmethyl)-1H-1,2,4- triazol-1-yl]-3- fluorobenzenesulfonamide | 480 | ¹H NMR (300 MHz, DMSO- d6) 2.26-2.46 (m, 2 H) 3.14- 3.24 (m, 1 H) 3.51-3.60 (m, 1 H) 3.77 (s, 2 H) 4.26 (s, 2 H) 4.63 (s, 2 H) 5.72 (d, J = 10.55 Hz,1 H) 5.92 (d, J = 10.36 Hz, 1 H) 7.19 (td, J = 8.57, 2.64 Hz, 1 H) 7.39- 7.47 (m, 2 H) 7.80 (s, 2 H) 7.86-8.03(m, 3 H) 10.84 (br. s., 1 H) | | 2.08 | 564 | 0.125 |
| F- 1c | 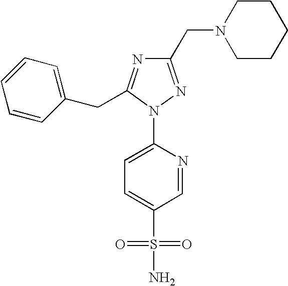 | 6-[5-benzyl-3- (piperidin-1-ylmethyl)- 1H-1,2,4-triazol-1- yl]pyridine-3- sulfonamide | 413 | ¹H NMR (300 MHz,DMSO- d₆) 1.29-1.47 (m, 1 H) 1.64- 1.92 (m, 5 H) 2.97-3.15 (m, 2 H) 3.64 (d, J = 11.9 Hz, 2 H) 4.49 (d, J = 4.3 Hz, 2 H) 4.73 (s, 2 H) 7.19-7.34 (m, 5 H) 7.81 (s,2 H) 8.08 (d, J = 8.7 Hz, 1 H) 8.47 (dd, J = 8.7, 2.2 Hz, 1 H) 8.97 (d, J = 2.2 Hz, 1 H) 10.41 (s, 1 H) | | 3.87 | 427 | |
| F- 1ca | 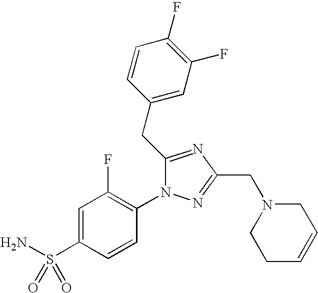 | 4-[5-(3,4- difluorobenzyl)-3-(3,6- dihydropyridin-1(2H)- ylmethyl)-1H-1,2,4- triaszol-3-yl]-3- fluorobenzenesulfon- amide | 464 | ¹H NMR (300 MHz, DMSO- d6) 2.25-2.48 (m, 2 H) 3.13- 3.29 (m, 1 H) 3.53-3.69 (m, 1 H) 3.82 (s, 2 H) 4.21 (s, 2 H) 4.55 (s,2 H) 5.74 (d, J = 10.7 Hz, 1 H) 5.93 (d, J = 10.38 Hz, 1 H) 7.00-7.07 (m, 1 H) 7.24- 7.42 (m, 2 H) 7.80 (s, 2 H) 7.85-7.89 (m, 3 H) 10.86 (br. s., 1 H) | | 2.45 | 1130 | 0.254 |
| F- 1cb | 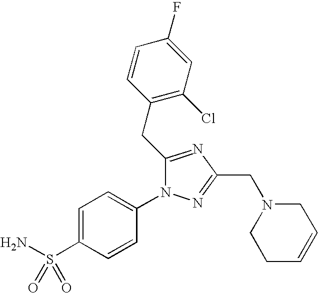 | 4-[5-(2-chloro-4- fluorobenzyl)-3-(3,6- dihydropyridin-1(2H)- ylmethyl)-1H-1,2,4- triazol-1- yl]benzenesulfon- amide | 462 | ¹H NMR (300 MHz, DMSO- d6) 2.25-2.48 (m, 2 H) 3.11- 3.23 (m, 1 H) 3.51-3.61 (m, 1 H) 3.77 (s, 2 H) 4.38 (s, 2 H) 4.51 (s, 2 H) 5.71 (d, J = 10.17 Hz, 1 H) 5.92 (d, J = 10.17 Hz, 1 H) 7.22 (td, J = 8.48, 2.64 Hz, 1 H) 7.38- 7.51 (m, 2 H) 7.64 (s, 2 H) 7.88 (d, J = 8.67 Hz, 2 H) 8.06 (d, J = 8.67 Hz, 2 H) 10.80 (br. s., 1 H) | | 2.41 | 923 | 1.14 |
| F- 1cc | 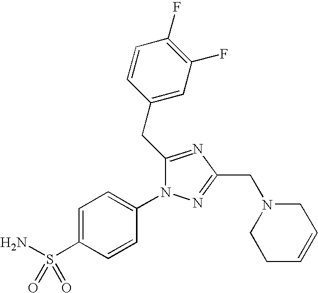 | 4-[5-(3,4- difluorobenzyl)-3-(3,6- dihydropyridin-1(2H)- ylmethyl)-1H-1,2,4- triazol-1- yl]benzenesulfon- amide | 446 | ¹H NMR (300 MHz, DMSO- d6) 2.26-2.49 (m, 2 H) 3.14- 3.27 (m, 1 H) 3.57-3.68 (m, 1 H) 3.82 (s, 2 H) 4.35 (s, 2 H) 4.52 (s, 2 H) 5.73 (d, J = 10.17 Hz, 1 H) 5.93 (d, J = 10.36 Hz, 1 H) 7.05-7.12 (m, 1 H) 7.30- 7.40 (m, 2 H) 7.63 (s, 2 H) 7.83 (d,J = 8.85 Hz, 2 H) 8.04 (d, J = 8.67 Hz, 2 H) 10.92 (br. s., 1 H) | | 3.55 | 1140 | |
| F- 1cd | 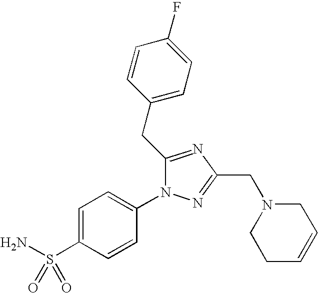 | 4-[3-(3,6- dihydropyridin-1(2H)- ylmethyl)-5-(4- fluorobenzyl)- 1H-1,2,4-triazol-1- yl]benzenesulfon- amide | 428 | ¹H NMR (300 MHz, DMSO- d6) d 2.32 (s, 2 H) 3.13-3.29 (m, 1 H) 3.55- 3.67 (m, 1 H) 3.82 (s, 2 H) 4.33 (s, 2 H) 4.52 (s, 2 H) 5.73 (d, J = 10.36 Hz, 1 H) 5.93 (d, J = 10.36 Hz, 1 H) 7.09-7.17 (m, 2 H) 7.19- 7.28 (m, 2 H) 7.63 (s, 2 H) 7.82 (d, J = 8.67 Hz, 2 H) 8.03 (d, J= 8.67 Hz, 2 H) 10.96 (br. s., 1 H) | | 2.89 | 879 | 1.24 |
| F- 1ce | 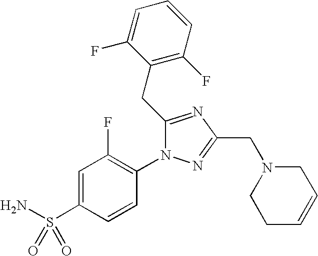 | 5-[5-(2,6- difluorobenzyl)-3-(3,6- dihydropyridin-1(2H)- ylmethyl)-1H-1,2,4- triazol-1-yl]-3- fluorobenzenesulfon- amide | 464 | ¹H NMR (300 MHz, DMSO- d6) 2.24-2.47 (m, 2 H) 3.11- 3.21 (m, 1 H) 3.50-3.59 (m, 1 H) 3.77 (s, 2 H) 4.21 (s,2 H) 4.53 (s, 2 H) 5.72 (d, J = 10.36 Hz, 1 H) 5.91 (d, J = 10.36 Hz, 1 H) 7.07 (t, J = 8.01 Hz, 2 H) 7.33-7.42 (m, 1 H) 7.81 (s, 2 H) 7.86- 7.95 (m, 2H) 8.01 (t, J = 7.72 Hz, 1 H) 10.81 (br. s., 1 H) | | | 558 | 0.042 |
| F- 1cf | 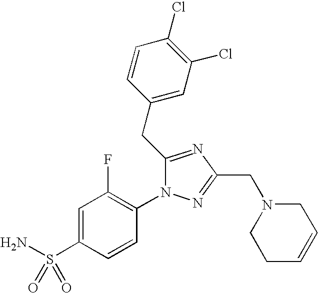 | 4-[5-(3,4- dichlorobenzyl)-3-(3,6- dihydropyridin-1(2H)- ylmethyl)-1H-1,2,4- triazol-1-yl]-3- fluorobenzenesulfon- amide | 496 | ¹H NMR (300 MHz, DMSO- d6) 2.24-2.47 (m, 2 H) 3.15- 3.28 (m, 1 H) 3.40-3.47 (m, 1 H) 3.82 (s, 2 H) 4.22 (s, 2 H) 4.55 (s, 2 H) 5.73 (d, J = 10.74 Hz, 1 H) 5.93 (d, J = 9.98 Hz, 1 H) 7.18-7.29 (m, 1 H) 7.49- 7.61 (m, 2 H) 7.80 (s, 2 H) 7.86-8.02 (m, 3 H) 10.78 (br. s., 1 H) | | | 203 | 0.096 |
| F- 1cg | 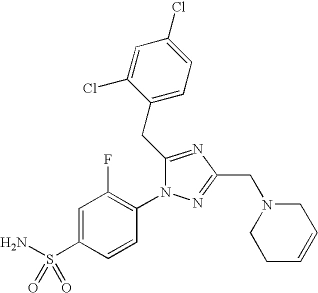 | 4-[5-(2,4- dichlorobenzyl)-3-(3,6- dihydropyridin-1(2H)- ylmethyl)-1H-1,2,4- triazol-1-yl]-3- fluorobenzenesulfon- amide | 496 | ¹H NMR (300 MHz, DMSO- d6) 2.22-2.48 (m, 2 H) 3.13- 3.23 (m, 1 H) 3.53-3.63 (m, 1 H) 3.76-3.80 (m, 2 H) 4.26 (s,2 H) 4.53 (s, 2 H) 5.72 (d, J = 10.36 Hz, 1 H) 5.92 (d, J = 10.36 Hz,1 H) 7.40-7.43 (m, 2 H) 7.61 (d, J = 1.70 Hz, 1 H) 7.80 (s, 2 H) 7.92 (td, J = 7.91, 1.70 Hz, 1 H) 7.96- 8.03 (m, 2 H) 10.79 (br. s., 1 H) | | | 279 | 0.03 |
| F- 1ch | 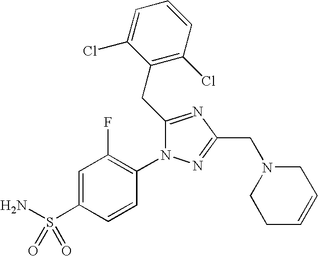 | 4-[5-(2,6- dichlorobenzyl)-3-(3,6- dihydropyridin-1(2H)- ylmethyl)-1H-1,2,4- triazol-1-yl]-3- fluorobenzenesulfon- amide | 496 | ¹H NMR (300 MHz, DMSO- d6) 2.24-2.48 (m, 2 H) 3.09- 3.20 (m, 1 H) 3.51-3.57 (m, 1 H) 3.72-3.81 (m, 2 H) 4.41 (s,2 H) 4.52 (s, 2 H) 5.71 (d, J = 10.17 Hz, 1 H) 5.91 (d, J = 10.55 Hz, 1 H) 7.15-7.22 (m, 1 H) 7.44- 7.52 (m, 2 H) 7.82 (s, 2 H) 7.88-8.00 (m, 2 H) 8.07(t, J = 7.0 Hz, 1 H) 10.78 (br. s., 1 H) | | | 356 | 0.077 |
| F- 1ci | 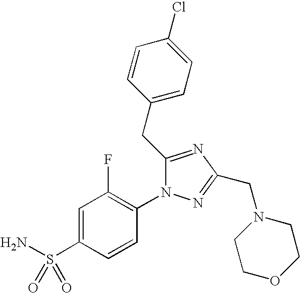 | 4-[5-(4-chlorobenzyl)- 3-(morpholin-4- yl)methyl)- 1H-1,2,4-triazol-1-yl]- 3- fluorobenzenesulfon- amide | 466 | ¹H NMR (300 MHz, DMSO- d6) 3.14-3.31 (m, 2 H) 3.46- 3.52 (m,2 H) 3.74-3.84 (m, 2 H) 3.93-4.02 (m, 2 H) 4.19 (s, 2 H) 4.53 (s, 2 H) 7.18 (d, J = 8.48 Hz,2 H) 7.35 (d, J = 8.48 Hz, 2 H) 7.79 (s, 2 H) 7.85-7.97 (m, 3 H) 11.04 (br. s., 1 H) | | | 442 | 0.176 |
| F- 1cj | 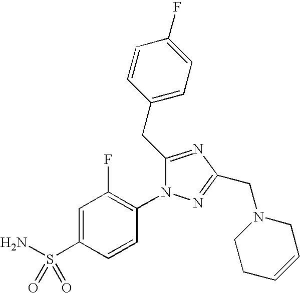 | 4-[3-(3,6- dihydropyridin-1(2H)- ylmethyl)- 5-(4-fluorobenzyl)- 1H-1,2,4-triazol-1-yl]- 3- fluorobenzenesulfon- amide | 446 | ¹H NMR (300 MHz, MeOH) 2.37-2.63 (m, 3 H) 3.67-3.72 (m, 1 H) 3.94- 3.97 (m, 2 H) 4.21 (s, 2 H) 4.60 (s, 2 H) 5.81 (d, J = 10.36 Hz, 1 H) 6.06 (d, J = 10.30 Hz, 1 H) 6.99 (t, J = 8.76 Hz, 2 H) 7.08-7.15 (m, 2 H) 7.74 (t, J = 7.50 Hz, 1 H) 7.86-7.94 (m, 2 H) | | | 625 | 0.241 |
| F- 1ck | 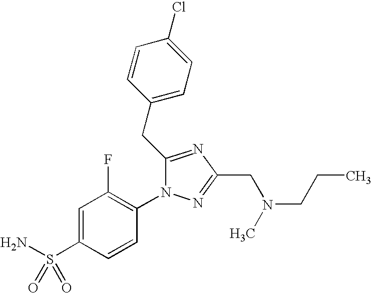 | 4-[5-(4-chlorobenzyl)- 3-{[methyl(propyl) amino]methyl}-1H- 1,2,4-triazol-1-yl]-3- fluorobenzenesulfon- amide | 452 | ¹H NMR (300 MHz, DMSO- d6) 0.89 (t, J = 7.35 Hz, 3 H) 1.67-1.82 (m, 2 H) 2.83 (d, J = 4.52 Hz, 3 H) 2.99-3.16 (m, 2 H) 4.19 (s,2 H) 4.47 (d, J = 2.45 Hz, 2 H) 7.18 (d, J = 8.29 Hz, 2 H) 7.35 (d, J = 8.48 Hz, 2 H) 7.80 (s, 2 H) 7.85-7.99 (m, 3 H) 10.68 (br. s., 1 H) | | | 271 | 0.059 |
| F- 1cl | 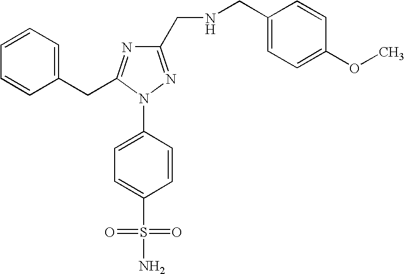 | 4-(5-benzyl-3-{[(4- methoxybenzyl)amino] methyl}-1H-1,2,4- triazol-1- yl]benzenesulfon- amide | 464 | ¹H NMR (300 MHz, DMSO- d₆) 3.78 (s, 3 H) 4.16-4.29 (m, 4 H) 4.34 (s, 2 H) 7.00 (d, J = 8.7 Hz, 2 H) 7.18 (d, J = 6.8 Hz, 2 H) 7.21- 7.35 (m, 3 H) 7.46 (d, J = 8.7 Hz, 2 H) 7.61 (s, 2 H) 7.78 (d, J = 8.7 Hz, 2 H) 8.02 (d, J = 8.7 Hz, 2 H) 9.71 (s, 2 H) | | 2.69 | 627 | 0.796 |
| F- 1c m | 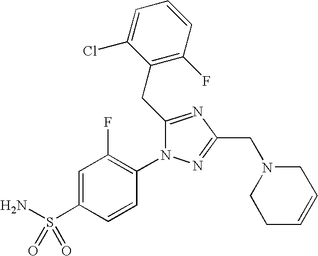 | 4-[5-(2-chloro-6- fluorobenzyl)-3- (3,6-dihydropyridin- 1(2H)-ylmethyl)-1H- 1,2,4-triazol-1-yl]-3- fluorobenzenesulfon- amide | 480 | ¹H NMR (300 MHz, DMSO- d6) 2.23-2.49 (m, 2 H) 3.05- 3.21 (m, 1 H) 3.52-3.60 (m, 1 H) 3.75-3.79 (m, 2 H) 4.29 (s, 2 H) 4.52 (s, 2 H) 5.71 (d, J = 10.36 Hz, 1 H) 5.91 (d, J = 9.98 Hz, 1 H) 7.15-7.26 (m, 1 H) 7.29- 7.40 (m, 2 H) 7.82 (s, 2H) 7.87-7.97 (m, 2 H)8.03 (t, J = 7.70 Hz, 1 H) 10.90 (br. s., 1 H) | | 398 | 0.026 | |
| F- 1cn | 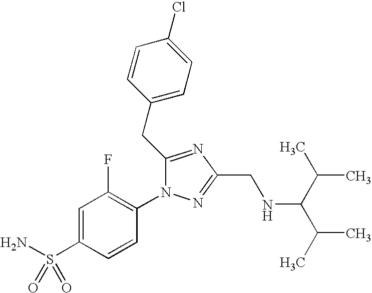 | 4-[5-(4-chlorobenzyl)- 3-{[(1-isopropyl-2- methylpropyl) amino]methyl}-1H- 1,2,4-triazol-1-yl]-3- fluorobenzenesulfon- amide | 494 | ¹H NMR (300 MHz, DMSO- d6) 0.93 (dd, J = 13.85, 6.88 Hz, 12 H) 2.01- 2.13 (m, 2 H) 2.80-2.87 (m, 1 H) 4.19 (s, 2 H) 4.32 (s, 2 H) 7.16 (d, J = 8.48 Hz, 2 H) 7.35 (d, J = 8.48 Hz, 2 H) 7.80 (s, 2 H) 7.87-7.97 (m, 3 H) 8.92 (br. s., 1 H) | | | 176 | 0.033 |
| F- 1co | 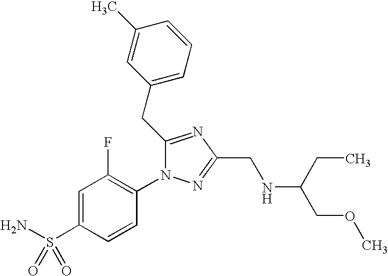 | 3-fluoro-4-[3-({[1- (methoxymethyl)propyl] amino}methyl)-5-(3- methylbenzyl)-1H- 1,2,4-triazol-1- yl]benzenesulfon- amide | 462 | ¹H NMR (300 MHz, DMSO- d6) 0.91 (t, J = 7.44 Hz, 3 H) 1.54-1.83 (m, 2 H) 2.22 (s, 3 H) 3.26- 3.37 (m, 4 H) 3.58-3.68 (m, 2 H) 4.13 (s, 2 H) 4.35 (s, 2 H) 6.64-6.88 (m, 2 H) 7.03 (d, J = 7.50 Hz, 1 H) 7.14 (t, J = 7.82 Hz,1 H) 7.81 (s, 2 H) 7.83-7.87(m, 2 H) 7.90 (d, J = 10.17 Hz, 1 H) 9.42 (br. s., 1 H) | | | 160 | 0.141 |
| F- 1cp | 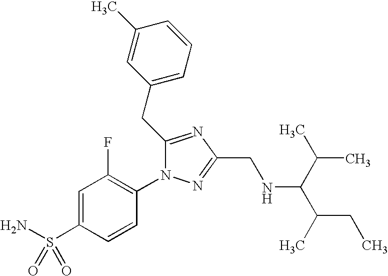 | 33-fluoro-4-[3-{[(1- isopropyl-2- methylpropyl) amino]methyl}-5-(3- methylbenzyl)-1H- 1,2,4-triazol-1- yl]benzenesulfon- amide | 474 | ¹H NMR (300 MHz, DMSO- d6) 0.88 (dd, J = 14.22, 6.88 Hz, 12 H) 1.92- 2.06 (m, 1 H) 2.74-2.80 (m, 1 H) 4.05 (s,2 H) 4.24 (s, 2 H) 6.73-6.77 (m, 2 H) 6.94 (d, J = 8.00 Hz,2 H) 7.05 (t, J = 7.82 Hz, 1 H) 7.72 (br. s., 2 H) 7.75-7.85 (m, 3 H) 8.91 (br. s., 1 H) | | | 100 | 0.06 |
| F- 1cq | 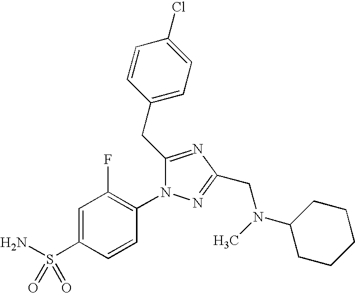 | 4-[5-(4-chlorobenzyl)- 3-{[cyclohexyl(methyl) amino]methyl}-1H- 1,2,4-triazol-1-yl]-3- fluorobenzenesulfon- amide | 492 | ¹H NMR (300 MHz, MeOH) 0.15-0.48 (m, 6 H) 0.60-0.70 (m, 2 H) 0.78- 0.91 (m, 2 H) 1.37-1.40 (m, 1 H) 1.65 (s, 3 H) 2.88 (s, 2 H) 3.15 (d, J = 14.25 Hz, 1 H) 3.29 (d, J = 14.25 Hz, 1 H) 5.79 (d, J = 8.29 Hz,2 H) 5.95 (d, J = 8.29 Hz,2 H) 6.44 (t, J = 7.44Hz, 1 H) 6.54-6.63 (m, 2 H) | | | 168 | 0.109 |
| F- 1cr | 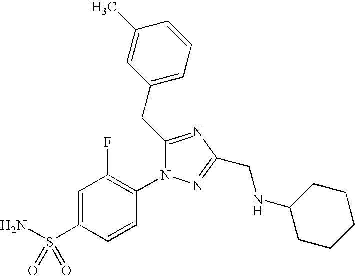 | 4-{3- [(cyclohexylamino)methyl]- 5-(3-methylbenzyl)- 1H-1,2,4-triazol-1-yl}- 3- fluorobenzenesulfon- amide | 458 | ¹H NMR (300 MHz, DMSO- d6) 1.04-1.45 (m, 5 H) 1.58- 1.66 (m, 1 H) 1.75-1.83 (m, 2 H) 2.05-2.13 (m, 2 H) 2.22 (s, 3 H) 3.03- 3.14 (m, 1 H) 4.13 (s, 2 H) 4.34 (s, 2 H) 6.84-6.87 (m, 2 H) 7.03 (d, J = 7.50 Hz, 1 H) 7.14 (t, J = 7.91 Hz, 1 H) 7.80 (s, 2 H) 7.84-7.87 (m, 2 H)7.90 (d, J = 9.98 Hz, 1 H) 9.39 (br. s., 1 H) | | | 88 | 0.152 |
| F- 1cs | 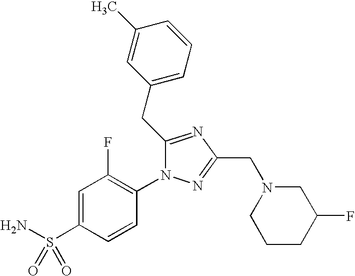 | 3-fluoro-4-{3-[(3- fluoropiperidin-1- yl)methyl]-5-(3- methylbenzyl)-1H- 1,2,4-triazol-1- yl}benzenesulfon- amide | 462 | ¹H NMR (300 MHz, DMSO- d₆) 1.38-1.87 (m, 4 H) 2.20 (s, 3 H) 2.32-2.47 (m, 1 H) 2.78- 2.94 (m, 1 H) 3.56-3.70 (m, 2 H) 4.07 (s, 2 H) 4.52-4.78 (m, J = 44.55, 3.67 Hz, 1 H) 6.80-6.85 (m, 2 H) 7.01 (d, J = 7.80 Hz, 1 H) 7.12 (t, J = 7.91 Hz, 1 H) 7.72 (br.s., 2 H) 7.77-7.88 (m, 3 H) 11.97 (br. s., 1 H) | | | | 0.042 |
| F- 1ct | 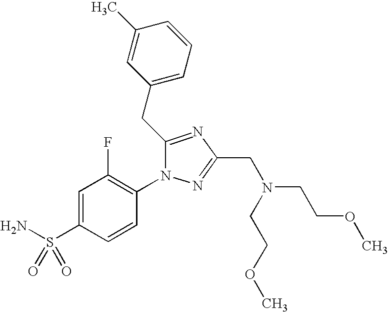 | 4-[3-{[bis-(2- methoxyethyl)amino] methyl}-5-(3- methylbenzyl)-1H- 1,2,4-triazol-1-yl]-3- fluorobenzenesulfon- amide | 492 | ¹H NMR (300 MHz, DMSO- d₆) 2.20 (s, 3 H) 2.74 (t, J = 6.22 Hz, 4 H) 3.22 (s, 6 H) 3.42 (t, J = 6.22 Hz, 4 H) 3.78 (s, 2 H) 4.07 (s, 2 H) 6.77-6.84 (m, 2 H) 7.00 (d, J = 7.70 Hz, 1 H) 7.11 (t, J = 7.82 Hz, 1 H) 7.72 (br. s., 2 H) 7.77-7.88 (m, 3H) | | | | 0.146 |
| F- 1cu | 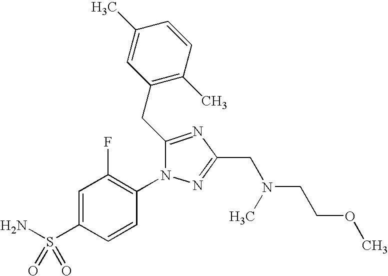 | 4-[5-(2,5- dimethylbenzyl)-3-{[(2- methoxyethyl)(methyl) amino]methyl}-1H- 1,2,4-triazol-1-yl]-3- fluorobenzenesulfon- amide | 460 (M − H)⁻ | ¹H NMR (300 MHz, DMSO- d₆) 1.93 (s, 1 H) 2.03 (s, 3 H) 2.12 (s, 3 H) 2.25 (s, 3 H) 2.60 (t, J = 6.03 Hz, 2 H) 3.23 (s, 3 H) 3.44 (t, J = 6.03 Hz, 2 H) 3.62 (s, 2 H) 4.06 (s, 2 H) 6.59 (s, 1 H) 6.89 (d, J = 7.50 Hz, 1 H)6.97 (d, J = 7.50 Hz, 1 H) 7.71 (s, 2 H) 7.74-7.87 (m, 3 H) | | | 50 | 0.076 |
| F- 1cv | 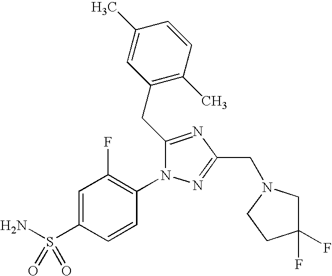 | 4-{3-[(3,3- difluoropyrrolidin-1- yl)methyl]-5-(2,5- dimethylbenzyl)-1H- 1,2,4-triazol-1-yl)}-3- fluorobenzenesulfon- amide | 480 | ¹H NMR (300 MHz, DMSO- d₆) 2.04 (s, 3 H) 2.12 (s, 3 H) 2.16-2.36 (m, 2 H) 2.82 (t, J = 6.97 Hz, 2 H) 3.01 (t, J = 13.56 Hz, 2 H) 3.74 (s, 2 H) 4.06 (s, 2 H) 6.59 (s, 1 H) 6.90 (dd, J = 7.50, 1.30 Hz, 1 H) 6.98 (d, J = 7.50 Hz, 1 H) 7.72(br. s., 2 H) 7.76- 7.92 (m, 3 H) | | | 43 | 0.048 |
| F- 1cw | 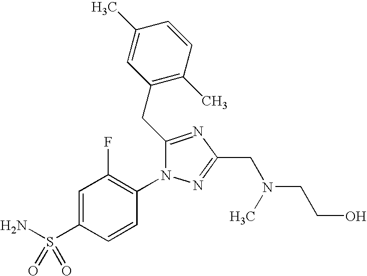 | 4-[5-(2,5- dimethylbenzyl)-3-{[(2- hydroxyethyl)(methyl) amino]methyl}-1H- 1,2,4-triazol-1-yl]-3- fluorobenzenesulfon- amide | 446 (M − H)⁻ | ¹H NMR (300 MHz, DMSO- d₆) 2.03 (s, 3 H) 2.12 (s, 3 H) 2.26 (s,3 H) 2.47-2.60 (m, 2 H) 3.51 (t, J = 6.40 Hz, 2 H) 3.63 (s,2 H) 4.05 (s, 2 H) 4.40 (br. s., 1 H) 6.59 (s,1 H) 6.89 (dd, J = 7.80, 1.20 Hz, 1 H) 6.97 (d, J = 7.80 Hz, 1 H) 7.71 (s, 2 H) 7.74-7.88 (m, 3 H) 11.96 (br. s., 1 H) | | | 43 | 0.038 |
| F- 1cx | 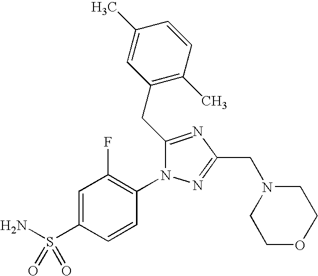 | 4-[5-(2,5- dimethylbenzyl)-3- (morpholin-4- ylmethyl)-1H-1,2,4- triazol-1-yl]-3- fluorobenzenesulfon- amide | 456 (M − H)⁻ | ¹H NMR (300 MHz, DMSO- d₆) 2.03 (s, 3 H) 2.12 (s, 3 H) 2.50-2.56 (m, 4 H) 3.53-3.63 (m, 6 H) 4.05 (s, 2 H) 6.60 (s, 1 H) 6.90 (d, J = 7.50 Hz, 1 H) 6.98 (d, J = 7.50 Hz, 1 H) 7.71 (s, 2 H) 7.75-7.87 (m, 3 H) | | | 55 | 0.158 |
| F- 1cy | 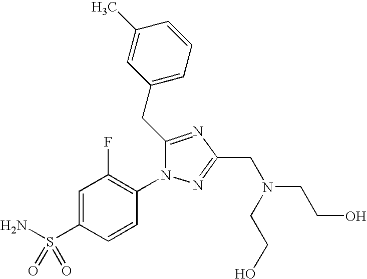 | 4-[3-{[bis(2- hydroxyethyl)amino] methyl}-5-(3- methylbenzyl)-1H- 1,2,4-triazol-1-yl]-3- fluorobenzenesulfon- amide | 464 | ¹H NMR (300 MHz, DMSO- d₆) 2.20 (s, 3 H) 2.68 (t, J = 6.12 Hz, 4 H) 3.44- 3.55 (m, J = 4.71 Hz, 4 H) 3.81 (s, 2 H) 4.07 (s, 2 H) 6.81 (br. s., 2 H) 7.00 (d, J = 7.50 Hz, 1 H) 7.12 (t, J = 7.82 Hz, 1 H) 7.71 (s, 2 H) 7.76-7.86 (m, 3 H) | | | 132 | 0.093 |
| F- 1cz | 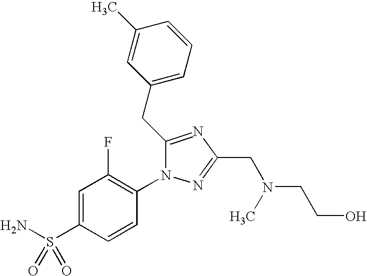 | 3-fluoro-4-[4-{[(2- hydroxyethyl)(methyl) amino]methyl}-5-(3- methylbenzyl)-1H- 1,2,4-triazol-1- yl]benzenesulfon- amide | 434 | ¹H NMR (300 MHz, DMSO- d₆) 2.20 (s,3 H) 2.28 (s, 3 H) 2.54-2.58 (m, 2 H) 3.48-3.56 (m, 2 H) 3.65 (s, 2 H), 4.06 (s, 2 H) 4.41 (br. s., 1 H) 6.79- 6.85 (m, 2 H) 7.00 (d, J = 7.72 Hz, 1 H) 7.12 (t, J = 7.82 Hz, 1 H) 7.72 (br. s., 3 H) 7.78-7.86 (m, 3 H) | | | 192 | 0.07 |
| F- 1d | 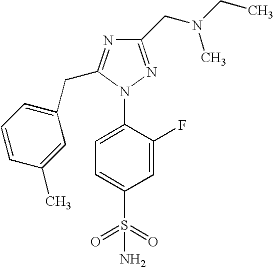 | 4-[3- {[ethyl(methyl)amino] methyl}-5-(3- methylbenzyl)-1H- 1,2,4-triazol-1-yl]-3- fluorobenzenesulfon- amide | 418 | ¹H NMR (300 MHz, DMSO- d₆) 1.26 (t, J = 7.2 Hz, 3 H) 2.22 (s,3 H) 2.73 (s, 3 H) 3.08 (q, J = 7.2 Hz, 2 H) 4.23 (s, 2 H) 4.38 (s, 2 H) 6.84- 6.87 (m, 2 H) 7.08 (d, J = 7.6 Hz, 1 H) 7.18 (t, J = 7.6 Hz, 1 H) 7.78 (s, 2 H) 7.87-7.93 (m, 3 H) | | | 146 | 0.252 |
| F- 1da | 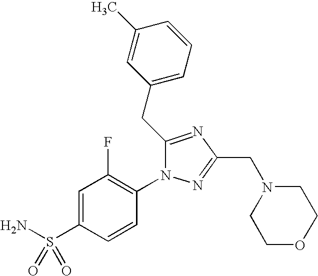 | 3-fluoro-4-[5-(2- methylbenzyl)-3- (morpholin-4- ylmethyl)-1H-1,2,4- triazol-1- yl]benzenesulfon- amide | 446 | ¹H NMR (300 MHz, DMSO- d₆) 2.11 (s, 3 H) 3.21-3.29 (m, 2 H) 3.43-3.53 (m, 2 H) 3.71- 3.83 (m, 2 H) 3.95-4.04 (m, 2 H) 4.16 (s, 2 H) 4.54 (s, 2 H) 6.92 (d, J = 7.35 Hz, 1 H)7.00- 7.09 (m,1 H) 7.13 (d, J = 3.98 Hz, 2 H) 7.79 (s, 2 H) 7.83- 7.98 (m, 3 H) 11.16 (br. s., 1 H) | | | 356 | 0.174 |
| F- 1db | 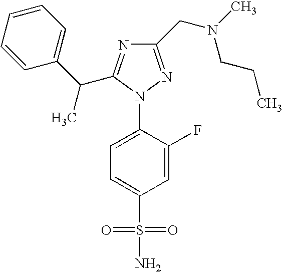 | 3-fluoro-4-[3- {[methyl(propyl)amino] methyl}-5-(1- phenylethyl)-1H-1,2,4- triazol-1- yl]benzenesulfonamide | 432 | ¹H NMR (300 MHz, DMSO- d6) 0.89 (t, J = 7.5 Hz, 3 H) 1.57-1.70 (m, 5 H), 2.55- 2.65 (m, 3 H), 2.71-2.84 (m, 2 H), 1.07- 4.18 (m, 2 H), 4.24 (q, J = 7.0 Hz, 1 H) 6.99- 7.04 (m, 2 H), 7.16-7.25 (m, 3 H), 7.70- 7.81 (m, 5 H). | | | 249 | 0.144 |
| F- 1dc | 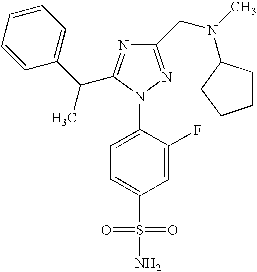 | 4-[3- {[cyclopentyl(methyl) amino]methyl}-5-(1- phenylethyl)-1H-1,2,4- triazol-1-yl]-3- fluorobenzenesulfon- amide | 458 | ¹H NMR (300 MHz, DMSO- d6) 1.45-1.74 (m, 9 H), 1.83- 2.00 (m, 2 H), 3.29-3.37 (m, 3 H), 3.67- 3.92 (m, 2 H), 4.24 (q, J = 7.0 Hz, 1 H), 6.95- 7.09 (m, 2 H), 7.17-7.25 (m, 3 H), 7.68- 7.81 (m, 5 H). | | | 231 | 0.106 |
| F- 1dd | 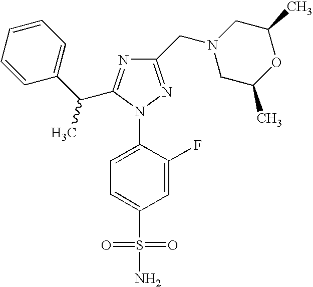 | 4-[3-{[(2R,6S)-2,6- dimethylmorpholin-4- yl]methyl}-5-(1- phenylethyl)-1H-1,2,4- triazol-1-yl]-3- fluorobenzenesulfon- amide | 474 | ¹H NMR (300 MHz, DMSO- d6) 1.03-1.08 (m, 6 H), 1.62 (d, J = 7.0 Hz, 3 H), 1.76- (1.85 (m, 2 H), 2.79-2.87 (m, 2 H), 3.54- 3.64 (m, 4 H), 4.19 (q, J = 7.0 Hz, 1 H), 6.98- 7.02 (m, 2 H), 7.14-7.25 (m, 3 H), 7.67- 7.76 (m, 5 H). | | | 517 | 0.644 |
| F- 1de | 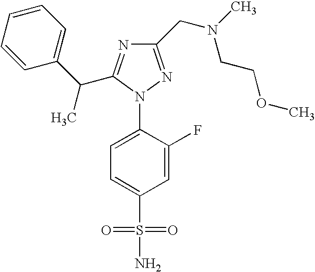 | 3-fluoro-4-[3-{[(2- methoxyethyl)(methyl) amino]methyl}-5-(1- phenylethyl)-1H-1,2,4- triazol-1- yl]benzenesulfon- amide | 448 | ¹H NMR (300 MHz, DMSO- d6) d 11.62 (d, J = 7.0 Hz, 3 H), 2.31-2.35 (m, 3 H), 2.66- 2.73 (m, 2 H), 3.25 (s, 3 H), 3.46-3.61 (m, 2 H), 3.71- 3.75 (m, 2 H), 4.20 (q, J = 7.0 Hz, 1 H), 6.97- 7.03 (m, 2 H), 7.15-7.25 (m, 3 H), 7.65- 7.78 (m, 5 H). | | | 410 | 0.123 |
| F- 1df | 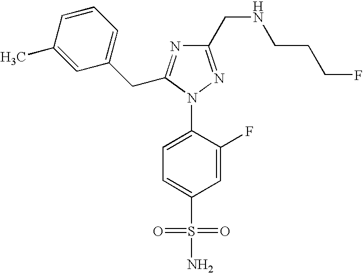 | 3-fluoro-4-[3-{[(3- fluoropropyl)amino] methyl}-5-(3- methylbenzyl)-1H- 1,2,4-triazol-1- yl]benzenesulfon- amide | 436 | ¹H NMR (300 MHz, DMSO- d6) 1.71-1.90 (m, 2 H), 2.19- 2.22 (m, 3 H), 2.66 (t, J = 7.0 Hz, 2 H), 3.74- 3.77 (m, 2 H) 4.04-4.07 (m, 2 H), 4.40- 4.45 (m, 1 H), 4.56-4.61 (m, 1 H), 6.80- 6.85 (m, 2 H), 6.98-7.03 (m, 1 H), 7.09- 7.15 (m, 1 H), 7.69-7.75 (m, 2 H), 7.78- 7.87 (m, 3 H). | | | 111 | 0.188 |
| F- 1dg | 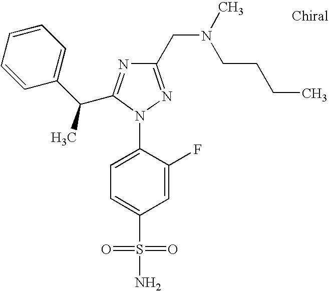 | 4-(3- {[butyl(methyl)amino] methyl}-5-[(1S)-1- phenylethyl]-1H-1,2,4- triazol-1-yl]-3- fluorobenzenesulfon- amide | 446 | ¹H NMR (300 MHz, DMSO-d6) 0.91 (t, J = 7.4 Hz, 3 H), 1.25- 1.40 (m, 2 H), 1.62-1.79 (m, 5 H), 2.85- 2.91 (m, 3 H), 3.07-3.22 (m, 2 H), 4.28 (q, J = 7.0 Hz, 1 H), 4.50-4.56 (m, 2 H), 6.99- 7.06 (m, 2 H), 7.18-7.26 (m, 3 H), 7.73- 7.83 (m, 5 H). | | | 114 | 0.121 |
| F- 1dh | 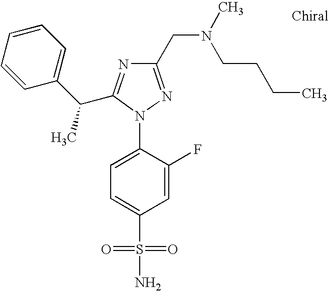 | 4-(3- {[butyl(methyl)amino] methyl}-5-[(1R)-1- phenylethyl]-1H-1,2,4- triazol-1-yl)-3- fluorobenzenesulfon- amide | 446 | ¹H NMR (300 MHz, DMSO- d6) d 0.91 (t, J = 7.4 Hz, 3 H), 1.25-1.40 (m, 2 H), 1.62- 1.79 (m, 5 H), 2.85-2.91 (m, 3 H), 3.07- 3.22 (m, 2 H), 4.28 (q, J = 7.0 Hz, 1 H) 4.50- 4.56 (m, 2 H), 6.99-7.08 (m, 2 H), 7.18- 7.26 (m, 3 H), 7.73-7.83 (m, 5 H). | | | 129 | 0.187 |
| F- 1di | 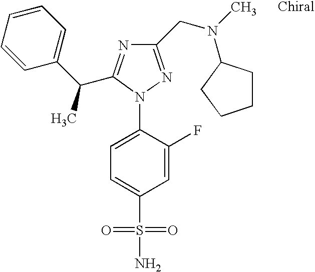 | 4-(3- {[cyclopentyl(methyl) amino]methyl}-5-[(1S)- 1-phenylethyl]-1H- 1,2,4-triazol-1-yl)-3- fluorobenzenesulfon- amide | 458 | ¹H NMR (300 MHz, DMSO- d6) 1.52-1.62 (m, 2 H), 1.63- 1.69 (m, 3 H), 1.71-1.88 (m, 4 H), 1.96- 2.25 (m, 2 H), 2.84-2.90 (m, 3 H), 3.55- 3.67 (m, 1 H), 4.24-4.34 (m, 1 H), 4.44- 4.63 (m, 2 H), 7.00-7.07 (m, 2 H), 7.17- 7.26 (m, 3 H), 7.72-7.83 (m, 5 H). | | | 176 | 0.159 |
| F- 1dj | 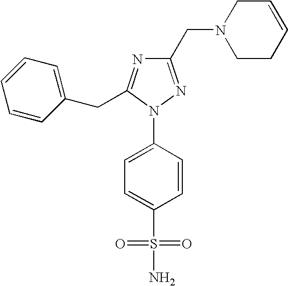 | 4-[5-benzyl-3-(3,6- dihydropyridin-1(2H)- ylmethyl)-1H-1,2,4- triazol-1- yl]benzenesulfon- amide | 410 | ¹H NMR (300 MHz, DMSO- d₆) 2.32-2.49 (m,2 H) 3.16- 3.30 (m, 1 H) 3.56-3.71 (m, 1 H) 3.77-3.89 (m, 2 H) 4.33 (s, 2 H) 4.54 (s, 2 H) 5.74 (d, J = 10.4 Hz, 1 H) 5.93 (d, J = 10.4 Hz, 1 H) 7.15-7.19 (m, 2 H) 7.21- 7.34 (m, 3 H) 7.61 (s, 2 H) 7.81 (d, J = 8.7 Hz, 2 H) 8.02 (d, J = 8.7 Hz, 2 H) 10.85 (s, 1 H) | | 2.30 | 424 | 0.575 |
| F- 1dk | 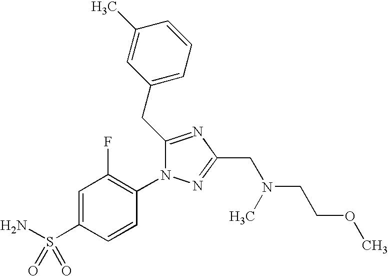 | 3-fluoro-4-[3-{[(2- methoxyethyl)(methyl) amino]methyl}-5-(3- methylbenzyl)-1H- 1,2,4-triazol-1- yl]benzenesulfon- amide | 448 | ¹H NMR (300 MHz, DMSO- d₆) 2.21 (s, 3 H) 2.88 (s, 3 H) 3.27-3.53 (m, 5 H) 4.14 (s, 2 H) 4.52 (s, 2 H) 6.81-6.89 (m, 2 H) 6.99-7.06 (m, 1 H) 7.14 (t, J = 7.82 Hz,1 H) 7.79 (s, 2 H) 7.81-7.96 (m, 3 H) 10.53 (br. s., 1 H) | | | 178 | 0.08 |
| F- 1dl | 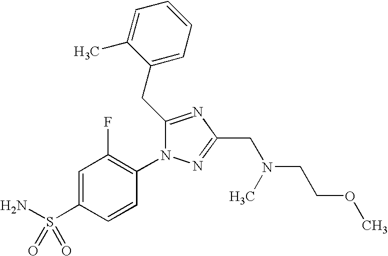 | 3-fluoro-4-[3-{[(2- methoxyethyl)(methyl) amino]methyl}-5-(2- methylbenzyl)-1H- 1,2,4-triazol-1- yl]benzenesulfon- amide | 448 | ¹H NMR (300 MHz, DMSO- d₆) 2.11 (s, 3 H) 2.86 (d, J = 3.58 Hz, 3 H) 3.30 (s, 3 H) 3.32- 3.52 (m, 2 H) 3.73 (t, J = 4.71 Hz, 2 H) 4.16 (s, 2 H) 4.50 (s, 2 H) 6.91 (d, J = 7.35 Hz, 1 H) 7.01-7.08 (m, 1 H) 7.13 (d, J = 3.96 Hz, 2 H) 7.80 (s, 2 H) 7.83-8.01 (m, 3 H) 10.64 (br. s., 1 H) | | | 380 | 0.111 |
| F- 1d m | 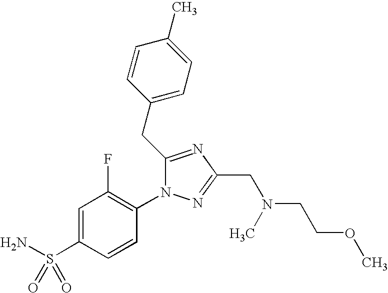 | 3-fluoro-4-[3-{[(2- methoxyethyl)(methyl) amino]methyl}-5-(4- methylbenzyl)-1H- 1,2,4-triazol-1- yl]benzenesulfon- amide | 448 | ¹H NMR (300 MHz, DMSO- d₆) 2.25 (s, 3 H) 2.87 (d, J = 3.20 Hz,3 H) 3.31 (s, 3 H) 3.35- 3.53 (m, 2 H) 3.74-3.77 (m, 2 H) 4.12 (s, 2 H) 4.48 (s, 2 H) 6.97 (d, J = 7.80 Hz, 2 H) 7.07 (d, J = 7.80 Hz, 2 H) 7.80 (s, 2 H) 7.82-7.93 (m, 3 H) 10.71 (br. s., 1 H) | | | 801 | 0.07 |
| F- 1dn | 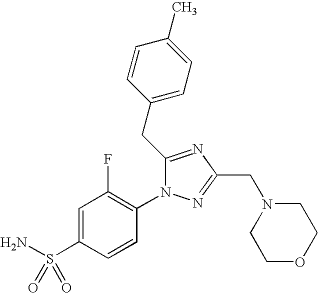 | 3-fluoro-4-[5-(4- methylbenzyl)-3- (morpholin-4- ylmethyl)-1H-1,2,4- triazol-1- yl]benzenesulfon- amide | 446 | ¹H NMR (300 MHz, DMSO- d₆) 2.25 (s, 3 H) 3.20-3.31 (m, 2 H) 3.46-3.50 (m, 2 H) 3.79- 3.86 (m, 2 H) 3.97-4.02 (m, 2 H) 4.24 (s, 2 H) 4.54 (s, 2 H) 6.97 (d, J = 7.80 Hz, 2 H) 7.07 (d, J = 7.80 Hz, 2 H) 7.79 (br. s., 2 H) 7.83- 7.94 (m, 3 H) 11.29 (br. s., 1 H) | | | 604 | 0.171 |
| F- 1e | 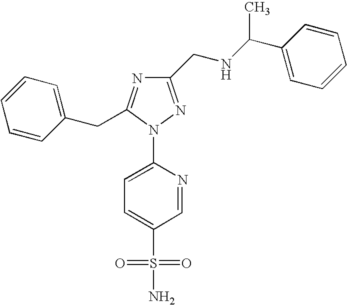 | 6-(5-benzyl-3-{[(1- phenylethyl)amino] methyl}-1H-1,2,4-triazol-1- yl)pyridine-3- sulfonamide | 449 | ¹H NMR (300 MHz, DMSO- d₆) 1.65 (d, J = 6.8 Hz, 3 H) 3.97-4.10 (m, 1 H) 4.17-4.30 (m, 1 H) 4.51- 4.63 (m, 1 H) 4.72 (s, 2 H) 7.20-7.33 (m, 5 H) 7.42-7.51 (m, 3 H) 7.58 (dd, J = 8.0, 1.3 Hz, 2 H) 7.80 (s, 2 H) 8.03 (d, J = 8.7 Hz, 1 H) 8.47 (dd, J = 8.7, 2.4 Hz, 1 H) 8.96 (d, J = 2.4 Hz, 1 H) 9.82 (s,1 H) 10.08 (s, 1 H) | | 2.70 | 139 | 1 |
| F-1f | 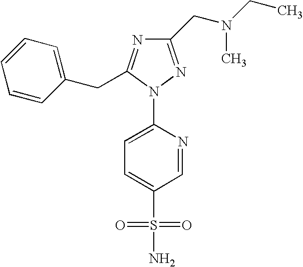 | 6-(5-benzyl-3- {[ethyl(methyl)amino] methyl}-1H-1,2,4- triazol-1-yl)pyridine-3- sulfonamide | 387 | ¹H NMR (300 MHz, DMSO- d₆) 1.30 (t, J = 7.2 Hz, 3 H) 2.85 (s, 3 H) 3.23 (q, J = 7.2 Hz, 2 H) 4.50 (s, 2 H) 4.73 (s, 2 H) 7.19-7.34 (m, 5 H) 7.81 (s, 2 H) 8.08 (d, J = 8.7 Hz, 1 H) 8.47 (dd, J = 8.7, 2.4 Hz, 1 H) 8.97 (d, J = 2.4 Hz, 1 H) 10.65 (s, 1 H) | | 2.90 | 391 | 1.26 |
| F- 1g | 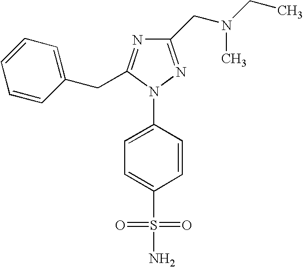 | 4-(5-benzyl-3- {[ethyl(methyl)amino] methyl}-1H-1,2,4- triazol-1- yl)benzenesulfon- amide | 386 | 1H NMR (300 MHz, DMSO- d6) 1.26 (t, J = 7.3 Hz, 3 H) 2.79 (s,3 H) 3.17 (q, J = 7.3 Hz, 2 H) 4.30 (s,2 H) 4.41 (s, 2 H) 7.02-7.19 (m, 2 H) 7.24 (m, 3 H) 7.58 (s, 2 H) 7.77 (d, J = 8.7 Hz,2 H) 7.99 (d, J = 8.7 Hz, 2 H) | | 3.03 | 550 | |
| F- 1h | 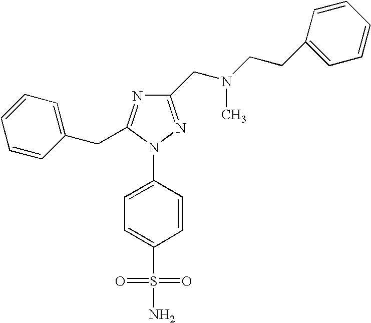 | 4-(5-benzyl-3- {[methyl(2- phenylethyl)amino] methyl}-1H-1,2,4-triazol-1- yl)benzenesulfon- amide | 462 | ¹H NMR (300 MHz, DMSO- d₆) 2.33 (s, 3 H) 2.62-2.72 (m,2 H) 2.73- 2.84 (m, 2 H) 3.68 (s, 2 H) 4.29 (s, 2 H) 7.11-7.16 (m, 2 H) 7.16-7.32 (m, 8 H) 7.55 (s,2 H) 7.74 (d, J = 8.7 Hz, 2 H) 7.97 (d, J = 8.7 Hz, 2 H) | | 2.21 | 183 | 0.408 |
| F-1i | 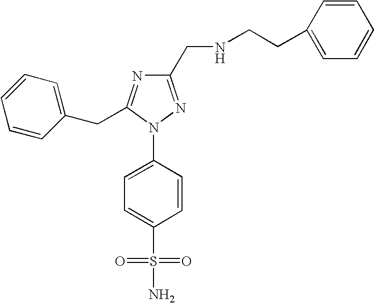 | 4-(5-benzyl-3-{[(2- phenylethyl)amino] methyl}-1H-1,2,4-triazol-1- yl)benzenesulfon- amide | 448 | ¹H NMR (300 MHz, DMSO- d₆) 2.98-3.06 (m, 2 H) 3.24- 3.33 (m,2 H) 4.33 (s, 2 H) 4.37 (s, 2 H) 7.16 (m, 2 H) 7.21-7.40 (m, 8 H) 7.60 (s,2 H) 7.78 (d, J = 8.1 Hz, 2 H) 8.02 (d, J = 8.1 Hz, 2 H) 9.57 (s,2 H) | | 2.90 | 295 | 0.987 |
| F-1j | 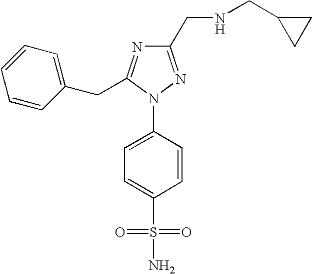 | 4-(5-benzyl-3- {[(cyclopropylmethyl) amino]methyl}-1H- 1,2,4-triazol-1- yl)benzenesulfon- amide | 398 | ¹H NMR (300 MHz, DMSO- d₆) 0.34-0.41 (m, 2 H) 0.54- 0.64 (m, 2 H) 1.02-1.16 (m, 1 H) 2.89-3.03 (m, 2 H) 4.33 (s, 4 H) 7.16 (d, J = 7.0 Hz, 2 H) 7.20-7.34 (m, J = 7.2 Hz, 3 H) 7.60 (s,2 H) 7.78 (d, J = 8.1 Hz, 2 H) 8.02 (d, J = 8.1 Hz, 2 H) 9.39 (s, 2 H) | | 3.64 | 402 | |
| F- 1k | 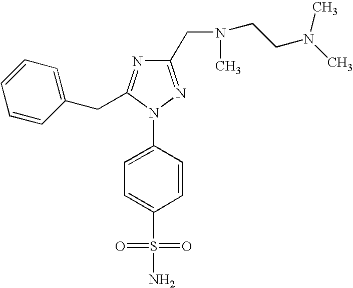 4-[5-benzyl-3-{[(2- (dimethylamino)ethyl] (methyl)amino}methyl)- 1H-1,2,4-triazol-1- yl]benzenesulfon- amide | 429 | ¹H NMR (300 MHz, DMSO- d₆) 2.15 (s, 3 H) 2.15 (s, 6 H) 2.52-2.97 (m, 4 H) 4.34 (s, 2 H) 4.42 (s, 2 H) 7.19 (d, J = 7.5 Hz, 2 H) 7.25- 7.37 (m, 3 H) 7.60 (s,2 H) 7.80 (d, J = 8.1 Hz, 2 H) 8.12 (d, J = 8.1 Hz, 2 H) | | 3.78 | 762 | | |
| F-1l | 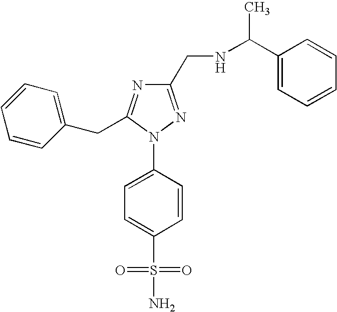 | 4-(5-benzyl-3-{[(1- phenylethyl)amino] methyl}-1H-1,2,4-triazol-1- yl)benzenesulfon- amide | 448 | ¹H NMR (300 MHz, DMSO- d₆) 1.65 (d, J = 6.8 Hz, 3 H) 3.97-4.05 (m, 1 H) 4.09-4.21 (m, 1 H) 4.32 (s, 2 H) 4.48- 4.62 (m, 1 H) 7.14-7.20 (m, 2 H) 7.21-7.34 (m, 3 H) 7.41- 7.60 (m, 3 H) 7.54-7.67 (m, 4 H) 7.76 (d, J = 8.7 Hz, 2 H) 8.02 (d, J = 8.7 Hz, 2 H) 9.87 (s, 1 H) 10.16 (s, 1 H) | | 2.81 | 285 | 0.763 |
| F- 1m | 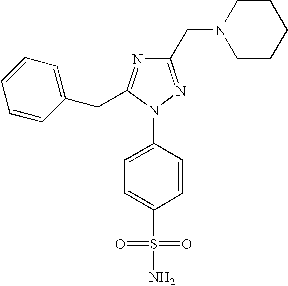 | 4-[5-benzyl-3- (piperidin-1-ylmethyl)- 1H-1,2,4-triazol-1- yl]benzenesulfon- amide | 412 | ¹H NMR (300 MHz, DMSO- d₆) 1.23-1.50 (m, 1 H) 1.57- 1.92 (m, 5 H) 2.89-3.15 (m, 2 H) 3.41-3.63 (m, 2 H) 4.32 (s, 2 H) 4.41 (s, 2 H) 7.09-7.20 (m, 2 H) 7.26 (s, 3 H) 7.60 (s, 2 H) 7.80 (d, J = 8.7 Hz, 2 H) 8.00 (d, J = 8.7 Hz,2 H) | | 2.92 | 508 | 1.34 |
| F- 1n | 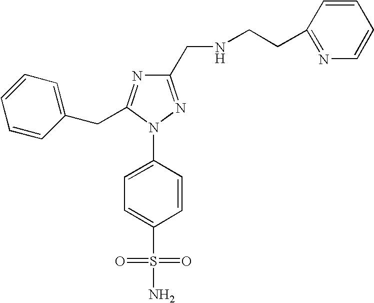 | 4-(5-benzyl-3-{[(2- pyridin-2- ylethyl)amino]methyl}- 1H-1,2,4-triazol-1- yl)benzenesulfon- amide | 449 | ¹H NMR (300 MHz, DMSO- d₆) 3.46-3.52 (m, 2 H) 3.55- 3.63 (m, 2 H) 4.34 (s, 2 H) 4.40 (s, 2 H) 7.14-7.20 (m, 2 H) 7.21-7.34 (m, 3 H) 7.62 (s, 2 H) 7.76- 7.82 (m, 3 H) 7.85 (d, J = 8.1 Hz, 1 H) 8.02 (d, J = 8.7 Hz, 2 H) 8.34 (t, J = 8.1 Hz, 1 H) 8.77 (d, J = 4.7 Hz, 1 H)9.83 (s, 1 H) | | 3.52 | 421 | |
| F- 1o | 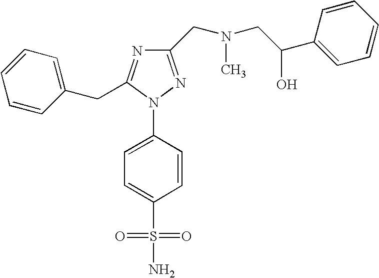 | 4-(5-benzyl-3-{[(2- hydroxy-2- phenylethyl)(methyl) aminp]methyl}-1H- 1,2,4-triazol-1- yl)benzenesulfon- amide | 478 | ¹H NMR (300 MHz, DMSO- d₆) 2.38 (s, 3 H) 2.61 (d, J = 7.0 Hz, 2 H) 3.66-3.82 (m, 2 H) 4.29 (s, 2 H) 4.70-4.80 (m, 1 H) 5.03 (d, J = 3.8 Hz, 1 H) 7.11-7.16 (m, 2 H) 7.19- 7.35 (m, 8 H) 7.55 (s, 2 H) 7.73 (d, J = 8.7 Hz,2 H) 7.97 (d, J = 8.7 Hz, 2 H) | | 2.46 | 197 | 0.419 |
| F- 1p | 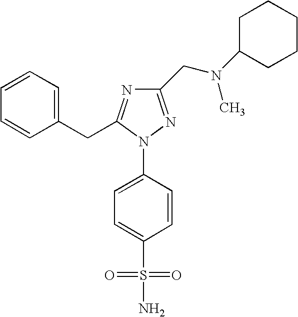 | 4-(5-benzyl-3- {[cyclohexyl(methyl) amino]methyl}-1H- 1,2,4-triazol-1- yl)benzenesulfonamide | 440 | ¹H NMR (300 MHz, DMSO- d₆) 1.04-1.34 (m, 5 H) 1.52- 1.63 (m, 1 H) 1.68-1.87 (m, 4 H) 2.25 (s, 3 H) 2.35-2.49 (m, 1 H) 3.66 (s, 2 H) 4.28 (s, 2 H) 7.10-7.15 (m, 2 H) 7.19- 7.33 (m, J = 7.2 Hz, 3 H) 7.54 (s, 2 H) 7.73 (d, J = 8.7 Hz, 2 H) 7.97 (d, J = 8.7 Hz, 2 H) | | 2.58 | 304 | 0.254 |
| F- 1q | 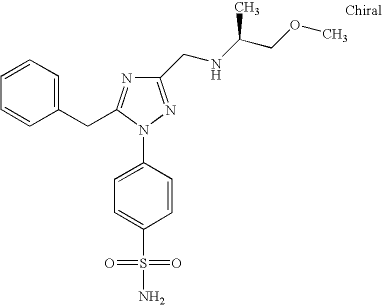 | 4-[5-benzyl-3-({[(1S)- 2-methoxy-1- methylethyl]amino} methyl)-1H-1,2,4-triazol-1- yl]benzenesulfonamide | 416 | ¹H NMR (300 MHz, DMSO- d₆) 0.98 (d, J = 6.4 Hz, 3 H) 2.83-2.95 (m, 1 H) 3.24 (s, 3 H) 3.74 (d, J = 14.1 Hz 1 H) 3.84 (d, J = 14.1 Hz 1 H) 4.28 (s, 2 H) 7.11- 7.16 (m, 2 H) 7.19-7.32 (m, 3 H) 7.55 (s, 2 H) 7.74 (d, J = 8.7 Hz, 2 H) 7.97 (d, J = 8.7 Hz, 2 H) | | 4.28 | 846 | |
| F- 1r | 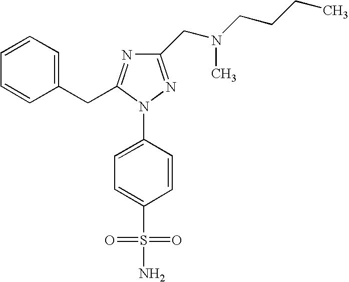 | 4-(5-benzyl-3- {[butyl(methyl)amino] methyl}-1H-1,2,4- triazol-1- yl)benzenesulfonamide | 414 | ¹H NMR (300 MHz, DMSO- d₆) 0.87 (t, J = 7.3 Hz, 3 H) 1.21-1.36 (m, 2 H) 1.38-1.51 (m, 2 H) 2.40 (t, J = 7.4 Hz, 2 H) 3.58 (s, 2 H) 4.29 (s,2 H) 7.12 (d, J = 6.8 Hz, 2 H) 7.18- 7.33 (m, 3 H) 7.73 (d, J = 8.5 Hz, 2 H) 7.97 (d, J = 8.5 Hz, 2 H) | | 2.69 | 355 | 0.379 |
| F- 1s | 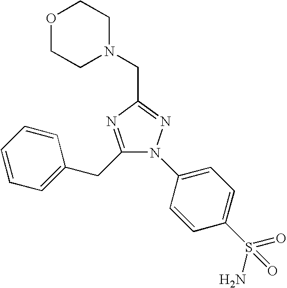 | 4-[5-benzyl-3- (morpholin-4- ylmethyl)-1H-1,2,4- triazol-1- yl]benzenesulfonamide | 414 | ¹H NMR (300 MHz, DMSO- d₆) 3.55-3.62 (m, 6 H) 4.28 (s, 2 H) 7.10- 7.17 (m, 2 H) 7.19-7.33 (m, 3 H) 7.55 (s, 2 H) 7.72-7.80 (d, J = 8.6 Hz, 2 H) 7.97 (d, J = 8.6 Hz, 2 H) | | 3.46 | 665 | |
| F-1t | 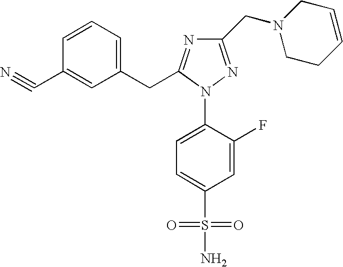 | 4-[5-(3-cyanophenyl)- 3-(3,6-dihydropyridin- 1(2H)-ylmethyl)-1H- 1,2,4-triazol-1-yl]-5- fluorobenzenesulfonamide | 453 | ¹H NMR (400 MHz, DMSO- d₆) 2.28-2.38 (m, 1 H) 2.39- 2.47 (m, 1 H) 3.13-3.29 (m, 1 H) 4.26 (s, 2 H) 4.55 (d, J = 3.3 Hz, 2 H) 5.72 (d, J = 89.9 Hz, 1 H) 5.92 (d, J = 9.9 Hz, 1 H) 7.48-7.59 (m, 2 H) 7.68- 7.77 (m, 2 H) 7.77 (s, 2 H) 7.86-7.98 (m, 3 H) | | | 1830 | 0.679 |
| F- 1u | 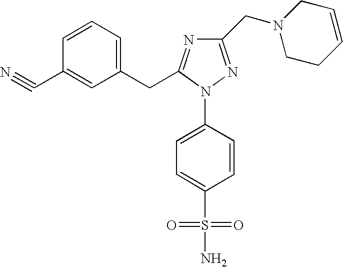 | 4-[5-(3-cyanobenzyl)- 3-(3,6-dihydropyridin- 1(2H)-ylmethyl)-1H- 1,2,4-triazol-1- yl]benzenesulfonamide | 435 | ¹H NMR (400 MHz, DMSO- d₆) 2.29-2.37 (m, 1 H) 2.38- 2.47 (m, 1 H) 3.17 (m, 1 H) 3.57-3.68 (m, 1 H) 4.38 (d, J = 13.9 Hz, 2 H) 4.53 (s, 2 H) 5.71 (d, J = 9.9 Hz, 1 H) 5.91 (d, J = 9.9 Hz, 1 H) 7.34-7.40 (m, 1H) 7.50- 7.63 (m, 3 H) 7.71-7.76 (m, 2 H) 7.83 (t, J = 9.0 Hz, 2H) 7.99-8.05 (m, 2 H) | | | 3590 | 3 |
| F- 1v | 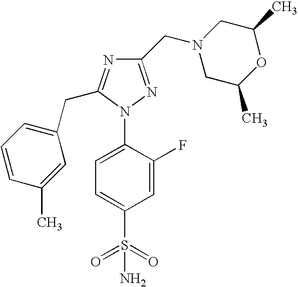 | 4-[3-{[(2R,6S)-2,6- dimethylmorpholin-4- yl]methyl}-5-(3- methylbemnzyl)-1H- 1,2,4-triazol-1-yl]-3- fluorobenzenesulfon- amide | 474 | ¹H NMR (300 MHz, DMSO- d₆) 1.15 (d, J = 6.4 Hz, 6 H) 2.22 (s, 3 H) 2.80 (t, J = 12.1 Hz, 2 H) 3.49 (d,J = 12.1 Hz, 2 H) 4.14 (s, 2 H) 4.51 (s, 2 H) 8.87 (d, J = 7.8 Hz, 2 H) 7.03 (d, J = 7.8 Hz, 1 H) 7.15 (t, J = 7.8 Hz, 1 H) 7.77 (s,2 H) 7.84-7.95 (m, 3 H) | | | 212 | 0.158 |
| F- 1w | 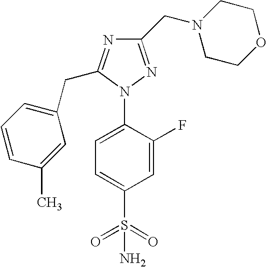 | 3-fluoro-4-[5-(3- methylbenzyl)-3- (morpholin-4- ylmethyl)-1H-1,2,4- triazol-1- yl]benzenesulfonamide | 446 | ¹H NMR (300 MHz, MeOH) 2.24 (s, 3 H) 3.36-3.50 (m, 2 H) 3.58-3.74 (m, 2 H) 3.77- 3.95 (m, 2 H) 4.06-4.21 (m, 2 H) 4.18 (s, 2 H) 4.60 (s, 2 H) 6.64 (br. s., 2 H) 7.03 (d, J = 7.8, 1 H) 7.07-7.15 (t, J = 7.8, 1 H) 7.65-7.73 (m, 1 H) 7.81-7.91 (m, 2 H) | | | 286 | 0.103 |
| F- 1x | 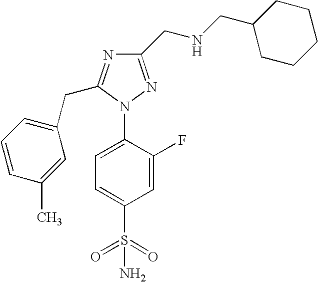 | 4-[3- {[(cyclohexylmethyl) aminp]methyl}-5-(3- methylbenzyl)-1H- 1,2,4-triazol-1-yl]-3- fluorobenzenesulfonamide | 472 | ¹H NMR (300 MHz, DMSO- d₆) 0.85-1.02 (m, 2 H) 1.09- 1.30 (m, 3 H) 1.72 (m, 6 H) 2.22 (s, 3 H) 2.84-2.96 (m, 2 H) 4.13 (s, 2 H) 4.33 (t, J = 5.2 Hz, 2 H) 6.83-6.89 (m, 2 H) 7.03 (d, J = 7.7 Hz, 1 H) 7.14 (t, J = 7.7 Hz, 1 H) 7.78 (s, 2 H) 7.84- 7.93 (m, 3 H) 9.30 (s, 2 H) | | | 62 | 0.088 |
| F- 1y | 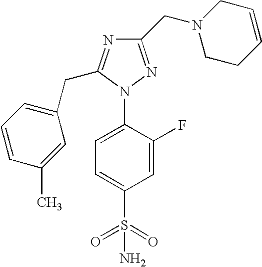 | 4-[3-(3,6- dihydropyridin-1(2H)- ylmethyl)-5-(3- methylbenzyl)-1H- 1,2,4-triazol-1-yl]-3- fluorobenzenesulfonamide | 442 | ¹H NMR (300 MHz, DMSO- d₆) 2.22 (s, 3 H) 3.81 (br. s.,=nl 2 H) 4.14 (s, 2 H) 4.54 (s, 2 H) 5.74 (d, J = 10.4 Hz, 1 H) 5.93 (d, J = 10.4 Hz, 1 H) 6.67 (d, J = 7.3 Hz, 2 H) 7.03 (d, J = 7.3 Hz, 1 H) 7.14 (t, J = 7.9 Hz, 1 H) 7.76 (s, 2 H) 7.83-7.94 (m, 3 H) | | | 134 | 0.259 |
| F- 1z | 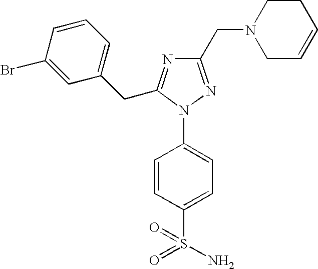 | 4-[5-(3-bromobenzyl)- 3-(3,6-dihydropyridin- 1(2H)-ylmethyl)-1H- 1,2,4-triazol-1- yl]benzenesulfonamide | 489 | ¹H NMR (400 MHz, DMSO- d₆) 2.04-2.10 (m, 2 H) 2.61 (t, J = 5.7 Hz, 2 H) 2.99 (d, J = 2.5 Hz, 2 H) 3.61 (s,2 H) 4.28 (s,2 H) 5.66 (q, J = 9.9 Hz, 2 H) 7.15- 7.19 (m, 1 H) 7.22-7.28 (m, 1 H) 7.39-7.44 (m, 2 H) 7.55 (s, 2 H) 7.76 (d, J = 8.8 Hz, 2 H) 7.97 (d, J = 8.6 Hz, 2H) | | 3.50 | 157 | |
| f-3a | 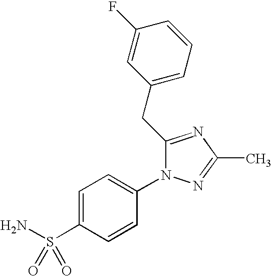 | 4-[5-(3-fluorobenzyl)- 3-methyl- 1H-1,2,4-triazol-1- yl]benzenesulfonamide | 347 | ¹H NMR (300 MHz, DMSO- d6) 2.32 (s, 3 H) 4.29 (s, 2 H) 6.98-7.14 (m, 3 H) 7.29-7.39 (m, 1 H) 7.56 (br. s., 2 H) 7.76 (d, J = 8.67 Hz, 2 H) 7.98 (d, J = 8.67 Hz, 2 H) | | 3.11 | 765 | 0.781 |
| f-3b | 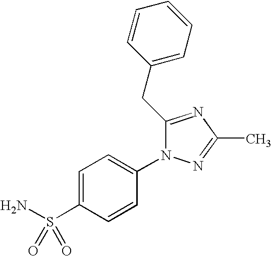 | 4-(5-benzyl-3-methyl- 1H-1,2,4-triazol-1-yl)- benzenesulfonamide | 329 | ¹H NMR (300 MHz, DMSO- d6) 2.33 (s, 3 H) 4.26 (s, 2 H) 7.15 (d, J = 6.59 Hz, 2 H) 7.21- 7.32 (m, 3 H) 7.58 (br. s., 2 H) 7.74 (d, J = 8.67 Hz, 2 H) 7.97 (d, J = 8.67 Hz, 2 H) | | 2.56 | 591 | 0.609 |
| f-3c | 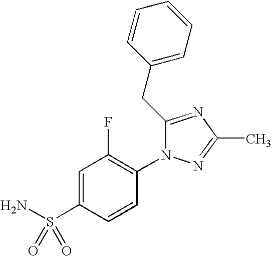 | 4-(5-benzyl-3-methyl- 1H-1,2,4-triazol-1-yl)- 3- fluorobenzenesulfonamide | 347 | ¹H NMR (300 MHz, DMSO- d6) 2.31 (s, 3 H) 4.05 (s, 2 H) 7.05 (d, J = 7.54 Hz, 2 H) 7.16- 7.27 (m, 3 H) 7.73 (br. s., 2 H) 7.77-7.89 (m, 3 H) | | 1.77 | 451 | 0.105 |
| f-3d | 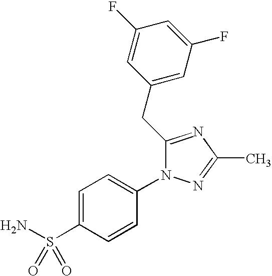 | 4-[5-(3,5- difluorobenzyl)-3- methyl-1H-1,2,4- triazol-1-yl] benzenesulfonamide | 365 | ¹H NMR (300 MHz, DMSO- d6) d 2.33 (s, 3 H) 4.31 (s, 2 H) 6.97 (d, J = 6.41 Hz, 2 H) 7.08- 7.18 (m, 1 H) 7.58 (br. s., 2 H) 7.77 (d, J = 8.67 Hz, 2 H) 7.99 (d, J = 8.48 Hz, 2 H) | | 2.79 | 1140 | 0.867 |
| f-3e | 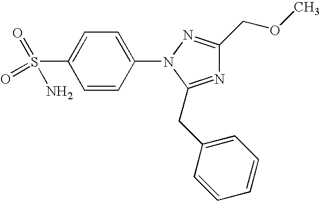 | 4-[5-benzyl-3- (methoxymethyl)-1H- 1,2,4-triazol-1- yl]benzenesulfon- amide | 359 | ¹H NMR (400 MHz, DMSO- d₆) 4.27 (s, 2 H) 4.43 (s, 2 H) 7.13 (d, J = 7.1 hz, 2 H) 7.19- 7.29 (m, 3 H) 7.54 (s, 2 H) 7.75 (d, J = 8.6 Hz, 2 H) 7.96 (d, J = 8.8 Hz, 2 H) | | 22.44 | 523 | 0.24 |
| f-3f | 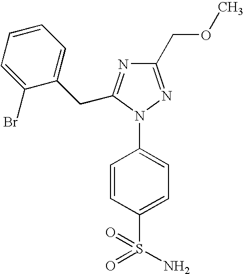 | 4-[5-(2-bromobenzyl)- 3-(methoxymethyl)- 1H-1,2,4-triazol-1- yl]benzenesulfon- amide | 438 | ¹H NMR (400 MHz, DMSO- d₆) 3.29 (s,3 H) 4.32 (s, 2 H) 4.39 (s, 2 H) 7.18-7.27 (m, 1 H) 7.32-7.41 (m, 2 H) 7.54- 7.62 (m, 3 H) 7.80-7.86 (m, 2 H) 7.97-8.04 (m, 2 H) | | 2.26 | 438 | 0.048 |
| f-3g | 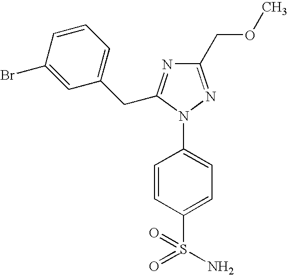 | 4-[5-(3-bromobenzyl)- 3-(methoxymethyl)- 1H-1,2,4-triazol-1- yl]benzenesulfon- amide | 439 | ¹H NMR (400 MHz, DMSO- d₆) 4.26 (s, 2 H) 4.42 (s, 2 H) 7.17 (d, J = 7.3 Hz, 1 H) 7.25 (t, J = 7.3 Hz, 1 H) 7.38-7.49 (m, 2 H) 7.56 (s, 2 H) 7.78 (d, J = 8.1 Hz, 2 H) 7.98 (d, J = 7.8 Hz, 2 H) | | 2.44 | 157 | 0.297 |
| f-3h | 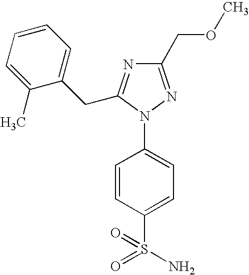 | 4-[3-(methoxymethyl)- 5-(2-methylbenzyl)- 1H-1,2,4-triazol-1- yl]benzenesulfonamide | 373 | ¹H NMR (400 MHz, DMSO- d₆) 2.11 (s, 3 H) 3.31 (s, 3 H) 4.22 (s, 2 H) 4.40 (s, 2 H) 6.93 (d, J = 7.6 Hz, 1 H) 7.04- 7.15 (m, 3 H) 7.56 (s, 2 H) 7.79 (d, J = 8.6 Hz, 2 H) 7.97 (d, J = 8.6 Hz, 2 H) | | 2.73 | 335 | 0.392 |
| f-3i | 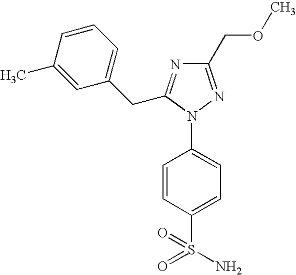 | 4-[3-(methoxymethyl)- 5-(3-methylbenzyl)- 1H-1,2,4-triazol-1- yl]benzenesulfonamide | 373 | ¹H NMR (400 MHz, DMSO- d₆) 2.22 (s, 3 H) 3.33 (s, 3 H) 4.21 (s, 2 H) 4.43 (s, 2 H) 8.87-8.93 (m, 2 H) 6.99-7.09 (m, 1 H) 7.13- 7.20 (m, 1 H) 7.55 (s, 2 H) 7.74 (d, J = 8.6 Hz, 2 H) 7.96 (d, J = 8.6 Hz, 2 H). | | 3.12 | 177 | 0.316 |
| f-3j | 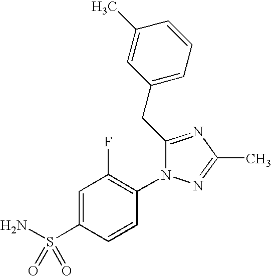 | 3-fluoro-4-[3-methyl-5- (3-methylbenzyl)-1H- 1,2,4-triazol-1- yl]benzenesulfonamide | 381 | ¹H NMR (300 MHz, DMSO- d₆) 2.24 (s, 3 H) 2.44 (s, 3 H) 4.10 (s, 2 H) 6.78 (s, 2 H) 7.01 (d, J = 7.50 Hz, 1 H) 7.10 (t, J = 7.82 Hz, 1 H) 7.57 (t, J = 7.63 Hz, 1 H) 7.82 (d, J = 8.10 Hz, 2 H) | | | 167 | 0.151 |
| f-3k | 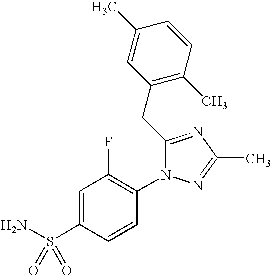 | 4-[5-(2,5- dimethylbenzyl)-3- methyl-1H-1,2,4- triazol-1-yl]-3- fluorobenzenesulfonamide | 375 | ¹H NMR (300 MHz, DMSO- d₆) 2.06 (s, 3 H) 2.16 (s, 3 H) 2.42 (s, 3 H) 4.12 (s, 2 H) 6.61 (s, 1 H) 6.90 (d, J = 7.80 Hz, 1 H) 6.98 (d, J = 7.60 Hz, 1 H) 7.57 (dd, J = 8.57, 7.06 Hz, 1 H) 7.71- 7.85 (m, 2 H) | | | 61 | 0.067 |
| f-4 | 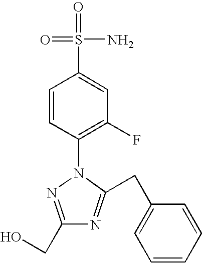 | 44-[5-benzyl-3- (hydroxymethyl)-1H- 1,2,4-triazol-1-yl]-3- fluorobenzenesulfonamide | 463 | ¹H NMR (400 MHz, MeOH- D4) 5.68 (s, 2H) 8.44 (m, 2 H) 8.64 (m, 3 H) 9.08 (t, J = 8 Hz,1H) 9.27 (m, 2 H) | | 3.57 | 528 | 0.133 |
| f-4a | 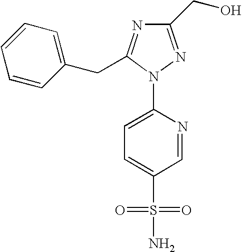 | 6-[5-benzyl-3- (hydroxymethyl)-1H- 1,2,4-triazol-1- yl]pyridine-3- sulfonamide | 346 | ¹H NMR (300 MHz, DMSO- d₆) 4.52 (s, 2 H) 4.69 (s, 2 H) 7.17-7.26 (m, 1 H) 7.28-7.33 (m, 4 H) 7.74 (s, 2 H) 8.05 (d, J = 8.7, 1 H) 8.41 (dd, J = 8.7, 2.2 Hz, 1 H) 8.93 (d, J = 2.2 Hz, 1 H) | | 1.99 | 251 | 0.498 |
| f-4b | 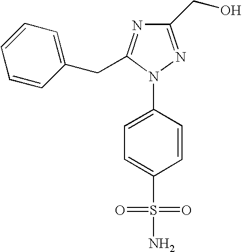 | 4-[5-benzyl-3- (hydroxymethyl)-1H- 1,2,4-triazol-1- yl]benzenesulfonamide | 345 | ¹H NMR (400 MHz, MeOD) 4.50 (s,2 H) 4.85 (s, 2 H) 7.10-7.19 (m, 2 H) 7.27-7.40 (m, 3 H) 7.75 (d,J = 8.7 Hz, 2 H) 8.11 (d, J = 8.7 Hz, 2 H) | | 1.79 | 472 | 0.176 |
| f-4c | 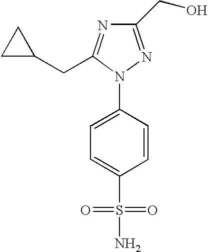 | 4-[5- (cyclopropylmethyl)-1H- 1,2,4-triazol-1- yl]benzenesulfonamide | 309 | ¹H NMR (300 MHz, DMSO- d₆) 0.02-0.04 (m, 2 H) 0.27- 0.38 (m,2 H) 0.85-1.02 (m, 1 H) 2.68 (d, J = 7.0 Hz, 2 H) 4.39 (s,2 H) 7.44 (s, 2 H) 7.66 (d, J = 8.7 Hz, 2 H) 7.88 (d, J = 8.7 Hz, 2 H) | | 2.59 | | |
| f-4d | 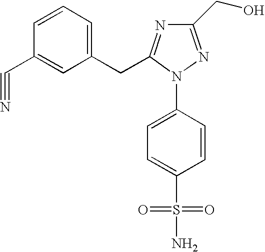 | 4-[5-(3-cyanobenzyl)- 3-(hydroxymethyl)-1H- 1,2,4-triazol-1- yl]benzenesulfonamide | 370 | ¹H NMR (400 MHz, DMSO- d₆) 4.35 (s, 2 H) 4.46 (d, J = 6.1 Hz, 2 H) 5.38 (t, J = 6.1 Hz, 1 H) 7.50- 7.61 (m, 4 H) 7.71-7.75 (m, 2 H) 7.80 (d, J = 8.7 Hz, 2 H) 8.00 (d, J = 8.7 Hz, 2 H) | | 2.73 | 2060 | 0.327 |
| f-4e | 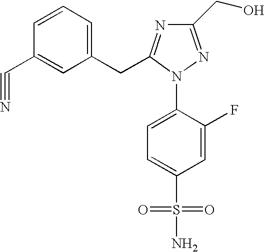 | 4-[5-(3-cyanobenzyl)- 3-(hydroxymethyl)-1H- 1,2,4-triazol-1-yl]-3- fluorobenzenesulfon- amide | 388 | ¹H NMR (400 MHz, DMSO- d₆) 4.18 (s, 2 H) 4.47 (d, J = 6.1 Hz, 2 H) 5.41 (t, J = 6.1 Hz, 1 H) 7.48- 7.56 (m, 2 H) 7.67 (s, 1 H) 7.70-7.74 (m, 3 H) 7.81-7.85 (m, 1 H) 7.86- 7.92 (m, 2 H) | | 2.32 | 1040 | 0.602 |
| f-4f | 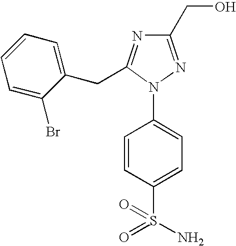 | 4-[5-(2-beomobenzyl)- 3-(hydroxymethyl)-1H- 1,2,4-triazol-1- yl]benzenesulfon- amide | 425 | ¹H NMR (400 MHz, DMSO- d₆) 4.31 (s, 2 H) 4.42 (d, J = 6.3 Hz, 2 H) 5.37 (t, J = 6.2 Hz, 1 H) 7.21 (ddd, J = 7.9, 6.4, 2.7 Hz, 1 H) 7.333-7.39 (m, 2 H) 7.53- 7.60 (m, 3 H) 7.78-7.84 (m, 2 H) 7.97-8.03 (m, 2 H) | | 2.96 | 418 | 0.129 |
| f-4g | 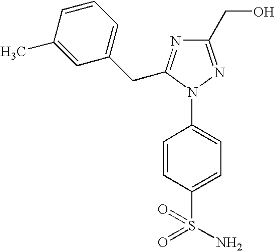 | 4-[3-(hydroxymethyl)- 5-(3-methylbenzyl)- 1H-1,2,4-triazol-1- yl]benzenesulfon- amide | 359 | ¹H NMR (400 MHz, DMSO- d₆) 2.18 (s, 3 H) 4.16 (s, 2 H) 4.42 (d, J = 6.1 Hz, 2 H) 5.34 (t, J = 6.1 Hz, 1 H) 6.84-6.90 (m, 2 H) 6.98 (d, J = 7.3 Hz, 1 H) 7.11 (t, J = 7.6 Hz, 1 H) 7.50 (s, 2 H) 7.67-7.72 (m, 2 H) 7.90-7.95 (m, 2 H). | | 2.26 | 140 | 0.406 |
| f-4h | 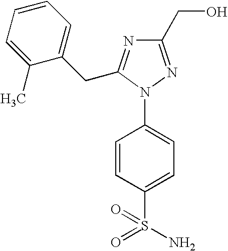 | 4-[3-(hydroxymethyl)- 5-(2-methylbenzyl)- 1H-1,2,4-triazol-1- yl]benzenesulfonamide | 359 | ¹H NMR (400 MHz, DMSO- d₆) 2.12 (s, 3 H) 4.21 (s, 2 H) 4.43 (d, J = 6.1 Hz, 2 H) 5.37 (t, J = 6.1 Hz,1 H) 6.94-7.12 (m, 1 H) 7.07- 7.16 (m, 3 H) 7.54 (s, 2 H) 7.67-7.72 (m, 2 H) 7.90-7.95 (m, 2 H). | | 2.20 | 306 | |
| f-4i | 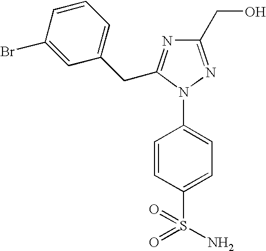 | 4-[5-(3-bromobenzyl)- 3-(hydroxymethyl)-1H- 1,2,4-triazol-1- yl]benzenesulfonamide | 425 | ¹H NMR (400 MHz, DMSO- d₆) 4.26 (s, 2 H) 4.45 (d, J = 6.1 Hz, 2 H) 5.39 (t, J = 6.1 Hz, 1 H) 7.16- 7.20 (m, 1 H) 7.22-7.28 (m, 1 H) 7.41-7.45 (m, 2 H) 7.55 (s, 2 H) 7.74- 7.79 (m, 2 H) 7.95-8.00 (m, 2 H). | | 2.40 | 96 | |
| f-4j | 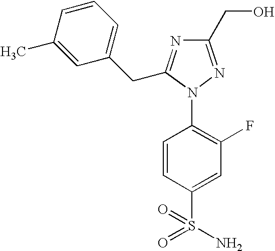 | 3-fluoro-4-[3- (hydroxymethyl)-5-(3- methylbenzyl)- 1H-1,2,4-triazol-1- yl]benzenesulfon- amide | 377 | ¹H NMR (300 MHz, DMSO- d6_) 2.21 (s, 3 H) 4.05 (s, 2 H) 4.48 (s, 2 H) 6.35 (br. s., 1 H) 6.77-6.91 (m, 2 H) 7.00- 7.04 (m, 1 H) 7.13 (t, J = 7.91 Hz, 1 H) 7.73 (s, 2 H) 7.77- 7.91 (m, 3 H) | | | 142 | 0.052 |
| f-4k | 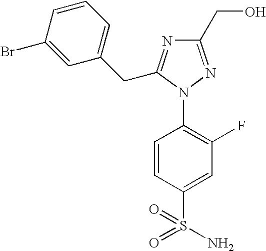 | 4-[5-(3-bromobenzyl)- 3-(hydroxymethyl)-1H- 1,2,4-triazol-1-yl]-3- fluorobznenesulfon- amide | 442 | ¹H NMR (400 MHz, MeOD) 4.13 (s, 2 H) 4.66 (s, 2 H) 6.98 (d, J = 7.6 Hz, 1 H) 7.11 (t, J = 7.8 Hz, 1 H) 7.20 (s,1 H) 7.34 (d, J = 8.1 Hz, 1 H) 7.61 (dd, J = 8.5, 7.0 Hz, 1 H) 7.82 (dt, J = 9.4, 2.5 Hz, 2 H). | | | 95 | 0.051 |
| f-4l | 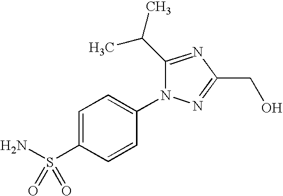 | 4-[3-(hydroxymethyl)- 5-isopropyl-1H-1,2,4- triazol-1- yl]benzenesulfonamide | 297 | ¹H NMR (300 MHz, DMSO- d6) 1.24 (d, J = 6.78 Hz,6 H) 3.13-3.24 (m, 1 H) 4.49 (s, 2 H) 7.57 (br. s., 2 H) 7.77 (d, J = 8.67 Hz, 2 H) 8.02 (d, J = 8.67 Hz, 2 H) | | 4.40 | | |
| f- 4m | 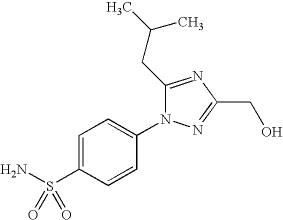 | 4-[3-(hydroxymethyl)- 5-isobutyl- 1H-1,2,4-triazol-1- yl]benzenesulfonamide | 311 | ¹H NMR (300 MHz, DMSO- d6) 0.87 (d, J = 6.78 Hz, 6 H) 2.00-2.14 (m,1 H) 2.76 (d, J = 7.16 Hz, 2 H) 4.53 (s, 2 H) 7.60 (br. s., 2 H) 7.80 (d, J = 8.67 Hz, 2 H) 8.03 (d, J = 8.48 Hz, 2 H) | | 6.09 | 4980 | |
| f-4n | 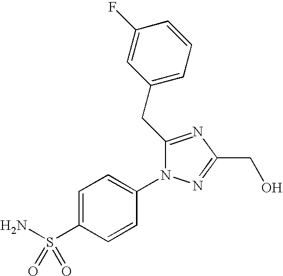 | 4-[5-(3-fluorobenzyl)- 3-(hydroxymethyl)- 1H-1,2,4-triazol-1- yl]benzenesulfon- amide | 363 | ¹H NMR (300 MHz, DMSO- d6) 4.32 (s, 2 H) 4.48 (s, 2 H) 6.98-7.13 (m, 3 H) 7.29-7.39 (m, 1 H) 7.58 (br. s., 2 H) 7.78 (d, J = 8.67 Hz, 2 H) 7.99 (d, J = 8.85 Hz, 2 H) | | 5.23 | 1250 | |
| f-4o | 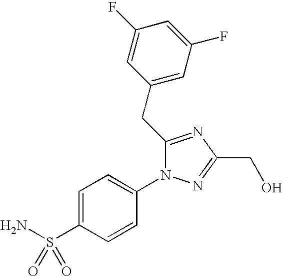 | 4-[5-(3,5- difluorobenzyl)-3- (hydroxymethyl)- 1H-1,2,4-triazol-1- yl]benzenesulfon- amide | 381 | ¹H NMR (300 MHz, DMSO- d6) 4.33 (s, 2 H) 4.48 (s,l 2 H) 6.95-7.02 (m, 2 H) 7.09-7.19 (m, 1 H) 7.58 (br. s., 2 H) 7.79 (d, J = 8.67 Hz, 2 H) 8.01 (d, J = 8.67 Hz, 2 H) | | 3.26 | 1460 | |
| f-4p | 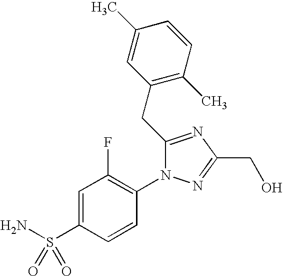 | 4-[5-(2,5- dimethylbenzyl)-3- (hydroxymethyl)-1H- 1,2,4-triazol-1-yl]-3- fluorobenzenesulfon- amide | 389 (M − H) | ¹H NMR (300 MHz, DMSO- d₆) 2.06 (s,3 H) 2.14 (s, 3 H) 4.04 (s, 2 H) 4.47 (d, J = 6.03 Hz, 2 H) 5.39 (t, J = 6.12 Hz, 1 H) 6.65 (s, 1 H) 6.91 (d, J = 7.70 hz, 1 H) 6.99 (d, J = 7.70 Hz, 1 H) 7.71 (s, 2 H) 7.78-7.90 (m, 3 H) | | | | 0.108 |
| f-4q | 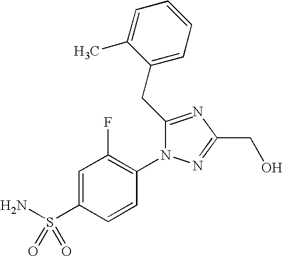 | 3-fluoro-4-[3- (hydroxymethyl)-5-(2- methylbenzyl)-1H- 1,2,4-triazol-1- yl]benzenesulfon- amide | 377 | ¹H NMR (300 MHz, DMSO- d₆) 2.11 (s, 3 H) 4.07 (s, 2 H) 4.46 (s, 2 H) 4.61 (br. s., 1 H) 6.91 (d, J = 7.35 Hz, 1 H) 6.99-7.19 (m, m3 H) 7.74 (br. s., 2 H) 7.78-7.91 (m, 3 H) | | | | 0.023 |
| f-4r | 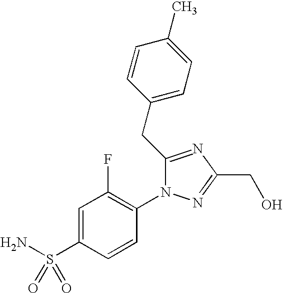 | 3-fluoro-4-[3- (hydroxymethyl)-5-(4- methylbenzyl)-1H- 1,2,4-triazol-1- yl]benzenesulfon- amide | 377 | ¹H NMR (300 MHz, DMSO- d₆) 2.25 (s, 3 H) 4.04 (s,2 H) 4.46 (s, 2 H) 6.96 (d, J = 7.75 Hz, 2 H) 7.06 (d, J = 7.75 Hz, 2 H) 7.73 (s, 2 H) 7.79-7.91 (m, 3 H) | | | 65 | 0.073 |
| G-1 | 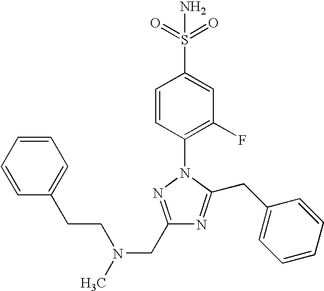 | 4-(5-benzyl-3- {[methyl(2- phenylethyl)amino] methyl}-1H-1,2,4-triazol-1- yl)-3- fluorobenzenesulfon- amide | 480 | ¹H NMR (400 MHz, MeOH- D4) 3.06 (s, 3H) 3.12 (t, J = 8 hz, 2H) 4.18 (2, 2H) 4.60 (bd, J = 4Hz, 2 H) 7.04 (m, 2 H) 7.19 (m, 3 H) 7.26 (m, 3H) 7.33 (m, 2H) 7.66 (t, J = 8 Hz, 1H) 7.84 (m,2 H) | | 2.77 | 110 | 0.127 |
| G- 1a | 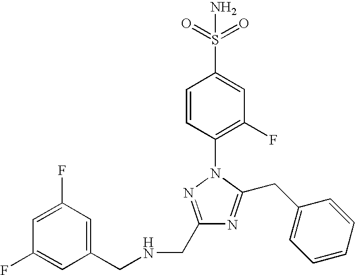 | 4-(5-benzyl-3-{[(3,5- difluorobenzyl)amino] methyl}-1H-1,2,4- triazol-1-yl)-3- fluorobenzenesulfon- amide | 488 | ¹H NMR (400 MHz, MeOH- d4) 4.16 (s, 2H) 4.38 (s, 2H), 4.41 (s, 2H) 7.04 (m, 2H) 7.09 (m, 1H), 7.15 (m, 2H), 7.19 (m, 3 H), 7.62 (t, J = 8 Hz, 1H), 7.83 (m, 2H). | | 2.95 | 165 | 0.118 |
| G- 1b | 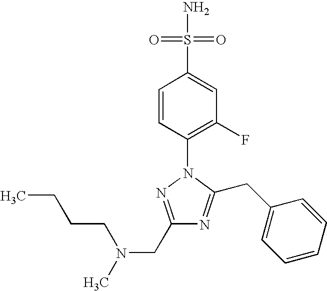 | 4-(5-benzyl-3- {[butyl(methyl)amino] methyl}-1H-1,2,4- triazol-1-yl)-3- fluorobenzenesulfon- amide | 432 | ¹H NMR (400 MHz, MeOH- d4) 0.98 (t,J = 8 Hz, 3H) 1.39 (m, 2H) 1.76 (m, 2H) 2.98 (s, 2H) 4.17 (s, 2H) 4.51 (q,m J1 = 16 Hz, J2 = 12 Hz, 2H) 7.03 (m, 2H) 7.19 (m, 3H) 7.65 (t, J = 8 Hz, 1 H) 7.84 (m, 2 H) | | 3.04 | 218 | 0.122 |
| G- 1c | 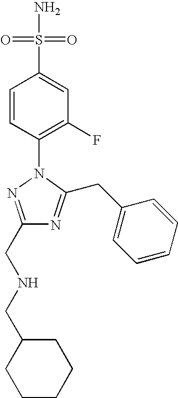 | 4-(5-benzyl-3- {[(cyclohexylmethyl) amino]methyl}-1H- 1,2,4-triazol-1-yl)-3- fluorobenzenesulfon- amide | 458 | ¹H NMR (400 MHz, MeOH- d4) 0.95 (q, J1 = 4 Hz, J2 = 8 Hz, 2H)1.24 (m, 4H) 1.53 (m, 1H) 1.75 (m, 4H) 2.58 9d, J = 8 Hz, 2H) 3.91 (s, 2H) 7.0 (m, 2H) 7.17 (m, 3H) 7.58 (t, J = 8 Hz, 1H) 7.82 (m, 2H). | | 2.30 | 156 | 0.22 |
| G- 1d | 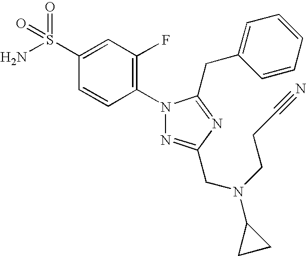 | 4-(5-benzyl-3-{[(2- cyanoethyl)(cyclo- propyl)amino]methyl}-1H- 1,2,4-triazol-1-yl)-3- fluorobenzenesulfon- amide | 455 | ¹H NMR (400 MHz, MeOH- d4) 0.50 (m, 4H) 2.03 (m, 1H) 2.66 (t, J = 8 Hz, 2H) 3.02 (t, J = 8 Hz, 2H) 3.94 (s, 2H) 4.14 (2, 2H) 6.97 (m, 2H) 7.16 (m, 3H) 7.56 (t, J = 8 Hz, 1H) 7.78 (m, 2H). | | 2.18 | 310 | 0.19 |
| G- 1e | 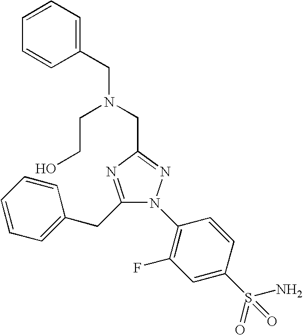 | 4-(5-benzyl-3- {[benzyl(2- hydroxyethyl)amino] methyl}-1H-1,2,4-triazol- 1-yl)-3- fluorobenzenesulfon- amide | 496 | ¹H NMR (400 MHz, MeOH- d4) 3.11 (t, J = 8 Hz, 2H) 3.79 (t, J = 8 Hz, 2H) 4.19 (s, 2H), 4.53 (s, 2H) 7.05 (m, 2H) 7.20 (m, 3H), 7.47 (m, 3 H) 7.59 (m, 2 H) 7.68 (t, J = 8 Hz, 1H) 7.84 (m, 2H) | | 2.61 | 250 | 0.058 |
| G- 1f | 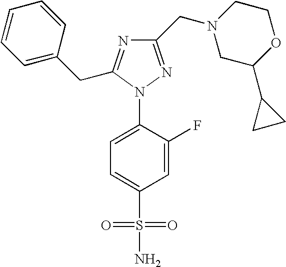 | 4-(5-benzyl-3-[(2- cyclopropylmorpholin- 44-yl)methyl]-1H-1,2,4- triazol-1-yl)-3- fluorobenzenesulfon- amide | 472 | ¹H NMR (400 MHz, MeOH-d4) 3.14 (m, 2H) 3.59 (m, 1H) 3.73 (m, 2H) 4.10 (m, 1H) 4.18 (s, 2H) 4.55 (bs, 2H) 6.85 (m, 2H) 7.0 (m, 3H) 7.48 9t, J = 8 Hz, 1H) 7.65 (m, 2H) | | | 480 | 0.178 |
| G- 1g | 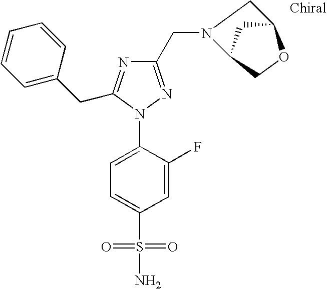 | 4-(5-benzyl-3-(2-oxa- 5- azabicyclo[2.2.1]hept- 5-ylmethyl)-1H-1,2,4- triazol-1-yl]-3- fluorobenzenesulfon- amide | 444 | ¹H NMR (400 MHz, MeOH- d4) 2.13 (bd, J = 12. Hz, 1H) 2.31 (bd, J = 12 Hz, 1H) 3.38 (bd, J = 12 Hz, 1H) 3.64 (bd, J = 12 Hz), 1H), 3.78 (bd, 8 Hz, 1H) 3.96 (bd, J = 8 hz, 1H) 4.08 (s, 2H) 4.60 (m, 4H) | | | 676 | 0.19 |
| G- 1h | 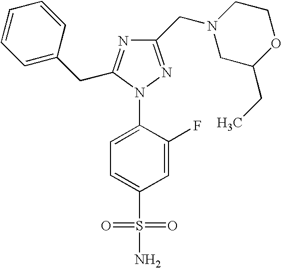 | 4-(5-benzyl-3-[(2- ethylmorpholin-4- yl)methyl]-1H-1,2,4- triazol-1-yl)-3- fluorobenzenesulfon- amide | 480 | ¹H NMR (400 Mhz, MeOH- d4) 0.34 (m, 1H) 0.44 (m, 1H) 0.57 (m, 2H) 0.91 (m, 1H) 3.13 (m, 2H) 3.57 (bd, J = 12 Hz, 1 H) 3.72 (m, 2H), 4.10 (q, J1 = 8 Hz, J2 = 4 Hz, 1 H) 4.18 (s, 2H) 4.54 (bs, 2H) 7.03 (m, 2H) 7.19 (m, 3H) 7.66 (q, J1 = 8 Hz, J2 = 2 Hz, 1H) 7.83 (m, 2H) | | | 397 | 0.152 |
| G- 1i | 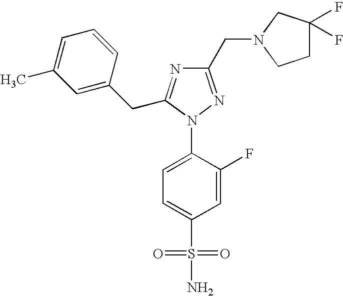 | 4-{3-[(3,3- difluoropyrrolidin-1- yl)methyl]-5-(3- methylbenzyl)-1H- 1,2,4-triazol-1-yl]-3- fluorobenzenesulfon- amide | 466 | ¹H NMR (400 MHz, MeOH- d4) 2.2 (s, 3H), 2.65-2.78 (m, 2H), 3.88-3.92 (t, J = 8 Hz, 2H), 4.05-4.11 (t, J = 12 Hz, 2H), 4.14 (s, 2H) 4.72 (s, 2H) 6.80 (bs, 2H) 6.96-7.0 (m, 1H) 7.05- 7.09 (m, 1H), 7.61-7.65 (t, J = 8 Hz, 1H), 7.80-7.84 (m, 2H) | | | 125 | 0.048 |
| G- 1j | 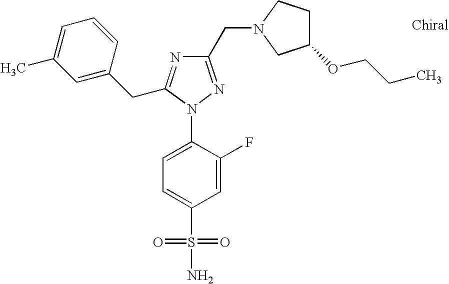 | 3-fluoro-4-(5-(3- methylbenzyl)-3- {[(3S)-3- propoxypyrrolidin-1- yl]methyl}-1H-1,2,4- triazol-1- yl)benzenesulfon- amide | 488 | ¹H NMR (400 MHz,MeOH- d4) 0.91-0.94 (t, J = 8 Hz, 3H), 1.56-1.61 (q, J = 8 Hz, 2H), 2.20 (s, 3H), 2.31 (m, 1H), 3.49-3.40 (m, 4H), 3.75-3.80 (m, 2H), 4.13 (s, 2H), 4.24 (bs, 1H), 4.57- 4.61 (m, 2H), 6.79 (bs, 2H), 6.98-6.99 (m, 1H), 7.05-7.09 (t, J = 8Hz, 1H), 7.60-7.64 (t, J = 8 Hz, 1H), 7.80-7.83 (m, 2H) | | | 196 | 0.083 |
| G- 1k | 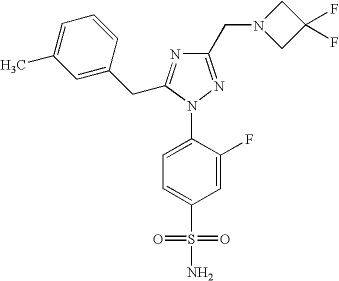 | 4-{3-[(3,3- difluoroazetidin-1- yl)methyl]-5-(3- methylbenzyl)-1H- 1,2,4-triazol-1-yl}-3- fluorobenzenesulfon- amide | 452 | ¹H NMR (400 MHz, MeOH- d4) 2.21 (s, 3H), 3.55-3.70 (m, 2H) 3.9- 4.02 (t, J = 12 Hz, 2H), 4.12 (bs, 2H), 4.31 (s, 2H), 6.75- 6.79 (m,2H), 6.97-6.99 (m, 1H), 7.03-7.08 (m, 1H), 7.56- 7.60 (t, J = 8 Hz, 1H), 7.80- 7.82 (m, 2H) | | | 83 | 0.168 |
| H-1 | 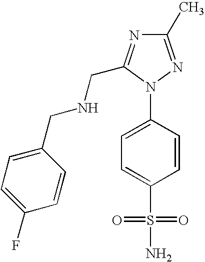 | 4-(5-{[(4- fluorobenzyl)amino] methyl}-3-methyl-1H- 1,2,4-triazol-1- yl)benzenesulfon- amide | 376 | ¹H NMR (300 MHz, DMSO- d₆) 2.34 (s, 3 H) 3.72 (s, 2 H) 3.82 (s, 2 H) 7.07-7.17 (m, 2 H) 7.26-7.34 (m, 2 H) 7.54 (s, 2 H) 7.89 (d, J = 8.9 Hz, 2 H) 7.98 (d, J = 8.9 Hz, 2 H) | | 9.47 | 2690 | |
| H- 1a | 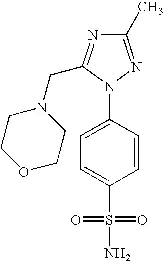 | 4-[3-methyl-5- (morpholin-4- ylmethyl)-1H-1,2,4- triazol-1- yl]benzenesulfon- amide | 338 | ¹H NMR (300 MHz, DMSO- d₆) 2.34 (s, 3- H) 2.45 (t, J = 4.3 Hz, 4 H) 3.51 (t, J = 4.3 Hz, 4 H) 3.65 (s, 2 H) 7.95 (d, J = 8.4 Hz, 2 H) 8.00 (d, J = 8.4 Hz, 2 H) | | 18.70 | | |
| H- 1b | 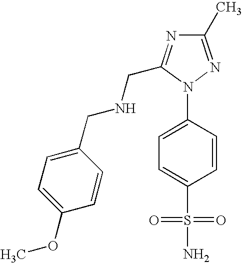 | 4-(5-{[(4- methoxybenzyl)amino] methyl}-3-methyl-1H- 1,2,4-triazol-1- yl)benzenesulfon- amide | 388 | ¹H NMR (300 MHz, DMSO- d₆) 2.34 (s, 3 H) 3.66 (s, 2 H) 3.74 (s,3 H) 3.79 (s, 2 H) 6.87 (d, J = 8.3 Hz, 2 H) 7.19 (d,J = 8.3 Hz, 2 H) 7.52 (s, 2 H) 7.89 (d, J = 8.7 Hz, 2 H) 7.95 (d, J = 8.7 Hz, 2 H) | | 6.60 | 1270 | |
| H- 1c | 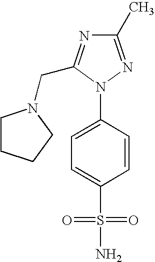 | 4-[3-methyl-5- (pyrrolidin-1-ylmethyl)- 1H-1,2,4-triazol-1- yl]benzenesulfon- amide | 322 | ¹H NMR (300 MHz, DMSO- d₆) 1.89-2.08 (m, 4 H) 2.43 (s, 3 H) 3.49- 3.71 (m, 4 H) 4.85 (s, 2 H) 7.61 (s, 2 H) 7.80 (d, J = 8.7 Hz, 2 H) 8.03 (d, J = 8.7 Hz, 2 H) | | 10.30 | 1850 | |
| H- 1d | 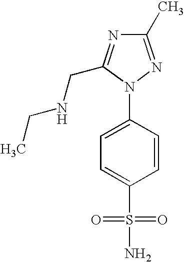 | 4-{5- [(ethylamino)methyl]- 3-methyl-1H-1,2,4- triazol-1- yl}benzenesulfon- amide | 296 | ¹H NMR (300 MHz, DMSO- d₆) 1.00 (t, J = 7.1 Hz, 3 H) 2.34 (s, 3 H) 2.55-2.63 (m, J = 7.1 Hz, 2 H) 2.85 (s, 2 H) 7.52 (s, 2 H) 7.93 (d, J = 8.8 Hz, 2 H) 7.98 (d, J = 8.8 Hz, 2 H) | | 5.40 | | |
| H- 1e | 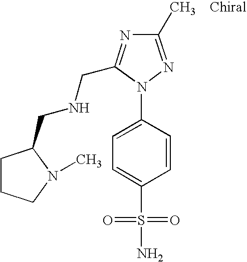 | 4-(3-methyl-5-[({[(2S)- 1-methylpyrrolidin-2- yl]methyl}amino)methyl}- 1H-1,2,4-triazol-1- yl}benzenesulfon- amide | 365 | ¹H NMR (300 MHz, DMSO- d₆) 2.43 (s, 3 H) 2.66 (s, 3 H) 4.61 (s, 2 H) 7.61 (s, 2 H) 7.86 (d, J = 8.5 Hz, 2 H) 8.04 (d, J = 8.5 Hz, 2 H) | | 134.00 | 4890 | |
| H- 1f | 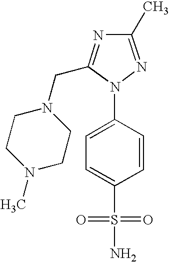 | 4-{3-methyl-5-[(4- methylpiperazin-1- yl)methyl]-1H-1,2,4- triazol-1- yl}benzenesulfon- amide | 351 | ¹H NMR (300 MHz, DMSO- d₆) 2.26 (s, 3 H) 2.38 (s, 3 H) 2.52-2.3.01 (m, 8H) 3.90 (s,2 H) 7.58 (s, 2 H) 7.88 (d, J = 8.6 Hz, 2 H) 8.02 (d, J = 8.6 Hz, 2 H) | | 86.70 | | |
| H- 1g | 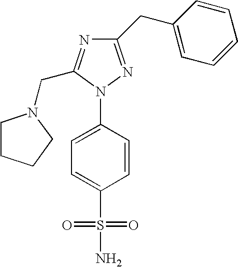 | 4-[3-benzyl-5- (pyrrolidin-1-ylmethyl)- 1H-1,2,4-triazol-1- yl]benzenesulfon- amide | 398 | ¹H NMR (300 MHz, DMSO- d₆) 1.68 (s, 4H) 3.76 (s,2 H) 4.08 (s, 2 H) 7.20-7.28 (m, 1H) 7.28-7.40 (m, 4 H) 7.52 (s, 2 H) 7.95 (d, J = 8.7 Hz, 2 H) 8.00 (d, J = 6.7 Hz, 2 H) | | 8.77 | 1640 | |
| H- 1h | 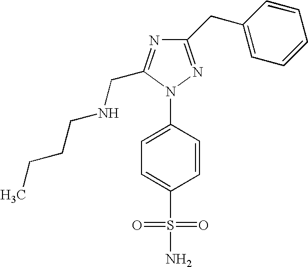 | 4-{3-benzyl-5- [(butylamino)methyl]- 1H-1,2,4-triazol-1- yl}benzenesulfon- amide | 400 | ¹H NMR (300 MHz, MeOD) 1.00 (t, J = 7.2 Hz, 3 H) 1.34- 1.57 (m, 2 H) 1.63-1.88 (m, 2 H) 3.28-3.44 (m, 1 H) 3.56- 3.85 (m, 1 H) 4.17 (s, 2 H) 4.62 (m, 2 H) 7.19-7.29 (m, 1 H) 7.33 (t, J = 7.4 Hz, 2 H) 7.40 (d, J = 7.4 Hz, 2 H) 7.81 (d, J = 7.9 Hz, 2 H) 8.14 (d, J = 7.9 Hz, 2 H) | | 2.52 | 1280 | 11.2 |
| H- 1i | 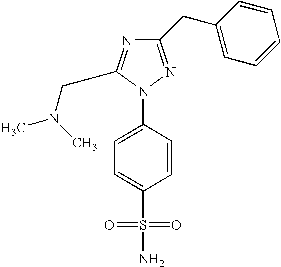 | 4-{3-benzyl-5- [(dimethylamino)methyl-] 1H-1,2,4-triazol-1- yl}benzenesulfon- amide | 372 | ¹H NMR (300 MHz, DMSO- d₆) 2.90 (s, 6 H) 4.15 (s, 2 H) 4.70 (s, 2 H) 7.18-7.29 (m, 1 H) 7.29-7.49 (m, 4 H) 7.66 (s, 2 H) 7.88 (d, J = 8.0 Hz, 2 H) 8.04 (d,J = 8.0 Hz, 2 H) | | 16.30 | 4530 | |
| H- 1j | 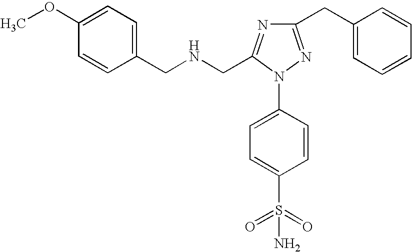 | 4-(3-benzyl-5-{[(4- methoxybenzyl)amino] methyl}-1H-1,2,4- triazol-1- yl)benzenesulfon- amide | 484 | ¹H NMR (300 MHz, DMSO- d₆) 3.65 (s, 2 H) 3.74 (s, 3 H) 3.81 (s, 2 H) 4.07 (s, 2 H) 6.86 (d, J = 7.7 hz, 2 H) 7.17 (d, J = 7.7 Hz, 2 H) 7.21-7.29 (m, 1 H) 7.29- 7.43 (m, 4 H) 7.56 (s, 2 H) 7.91 (d, J = 7.9 Hz, 2 H) 7.98 (d, J = 7.9 Hz, 2 H) | | 4.48 | 617 | |
| H- 1k | 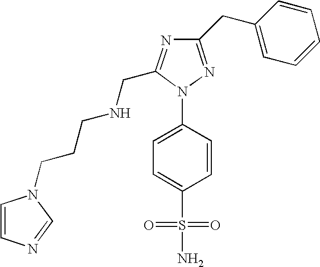 | 4-[3-benzyl-5-({[3-(1H- imidazol-1- yl)propyl]amino}methyl)- 1H-1,2,4-triazol-1- yl}benzenesulfon- amide | 452 | ¹H NMR (300 MHz, DMSO- d₆) 2.20-2.33 (m, 2 H) 2.99- 3.13 (m, 2 H) 4.13 (s, 2 H) 4.36 (t, J = 6.7 Hz, 2 H) 4.53 (s, 2 H) 7.22- 7.29 (m, 1 H) 7.31-7.40 (m, 4 H) 7.83 (s, 2 H) 7.73 (t, J = 1.6 Hz, 1 H) 7.82 (t, J = 1.6 Hz, 1 H) 7.87 (d, J = 8.9 Hz, 2 H) 8.03 (d, J = 8.9 Hz, 2 H) 9.24 (s, 1 H) | | 12.50 | | |
| H- 1l | 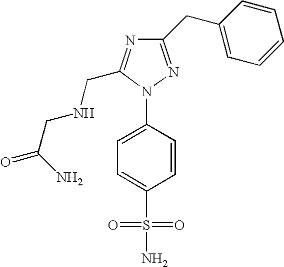 | N-2--({1-[4- (aminosulfonyl)phenyl]- 3-benzyl-1H-1,2,4- triazol-5- yl}methyl)glycinamide | 401 | ¹H NMR (300 MHz, DMSO- d₆) 4.56 (d, J = 2.6 Hz, 2 H) 7.13 (s, 1 H) 7.22-7.41 (m, 6 H) 7.47 (s, 1 H) 7.63 (s, 2 H) 7.85 (d, J = 8.5 Hz, 2 H) 8.03 (d, J = 8.5 Hz, 2 H) | | 12.30 | | |
| H- 1m | 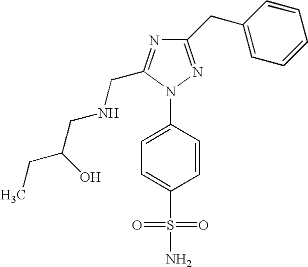 | 4-(3-benzyl-5-{[(2- hydroxybutyl)amino] methyl}-1H-1,2,4-triazol- 1- yl)benzenesulfon- amide | 416 | ¹H NMR (300 MHz, DMSO- d₆) 0.86 (t, J = 7.4 Hz, 3 H) 1.28-1.51 (m, 2 H) 2.87-3.03 (m, 1 H) 3.08- 3.23 (m, 1 H) 3.69-3.84 (m, 1 H) 4.14 (s, 2 H) 4.44-4.56 (m, 2 H) 7.21- 7.30 (m, 1 H) 7.29-7.42 (m, 4 H) 7.62 (s, 2 H) 7.85 (d, J = 8.6 Hz, 2 H) 8.03 (d, J = 8.6 Hz, 2 H) 9.23 (s, 1H) 9.59 (s, 1 H) | | 8.20 | | |
| H- 1n | 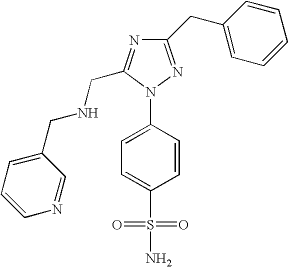 | 4-(3-benzyl-5- {[(pyridin-3- ylmethyl)amino]methyl}- 1H-1,2,4-triazol-1- yl)benzenesulfon- amide | 435 | ¹H NMR (300 MHz, DMSO- d₆) 4.13 (s, 2 H) 4.48 (s, 2 H) 4.59 (s, 2 H) 7.21-7.29 (m, 1 H) 7.29-7.44 (m, 4 H) 7.64 (s, 2 H) 7.87 (d, J = 8.5 Hz, 2 H) 7.89-7.97 (m, 1 H) 8.04 (d, J = 8.5 Hz, 2 H) 8.50 (d, J = 7.16 Hz, 1 H) 8.87 (d, J = 5.46 Hz, 1 H) 8.97 (s, 1 H) 10.41 (s, 1 H) | | 8.65 | | |
| H- 1o | 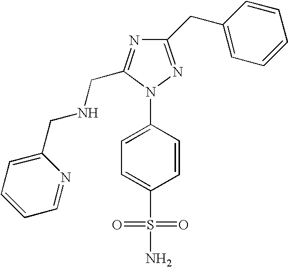 | 4-(3-benzyl-5- {[(pyridin-2- ylmethyl)amino]methyl}- 1H-1,2,4-triazol-1- yl)benzenesulfon- amide | 435 | ¹H NMR (300 MHz, DMSO- d₆) 4.14 (s, 2 H) 4.50 (s, 2 H) 4.61 (s, 2 H) 7.22-7.29 (m, 1 H) 7.30-7.41 (m, 4 H) 7.43- 7.53 (d, J = 7.91 Hz, 1 H) 7.63 (s,2 H) 7.64 (d, J = 8.7 Hz, 2 H) 7.88-7.96 (m, 1 H) 8.03 (d, J = 8.7 Hz, 2 H) 8.54-8.67 (m, 1 H) 10.06 (s, 1 H) | | 6.13 | 3870 | |
| H- 1p | 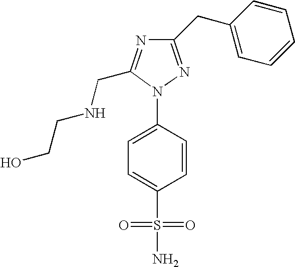 | 4-(3-benzyl-5-{[(2- hydroxyethyl)amino] methyl}-1H-1,2,4-triazol- 1- yl)benzenesulfon- amide | 388 | ¹H NMR (300 MHz, DMSO- d₆) 3.11-3.24 (m, 2 H) 3.70 (t, J = 5.3 Hz, 2 H) 4.14 (s, 2 H) 4.54 (t, J = 5.3 Hz, 2 H) 7.22- 7.29 (m, J = 6.78 Hz, 1 H) 7.30-7.41 (m, 4 H) 7.62 (s, 2 H) 7.85 (d, J = 8.6 Hz, 2 H) 8.03 (d, J = 8.5 Hz, 2 H) 9.45 (s, 2 H) | | 9.62 | | |
| H- 1q | 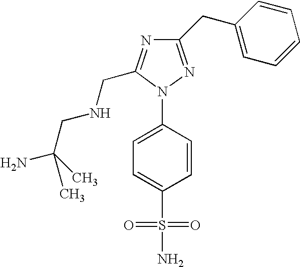 | 4-(5-{[(2-amino-2- methylpropyl)amino] methyl}-3-benzyl-1H- 1,2,4-triazol-1- yl)benzenesulfon- amide | 415 | ¹H NMR (300 MHz, DMSO- d₆) 1.41 (s, 6 H) 3.37 (s, 2 H) 4.15 (s, 2 H) 4.57 (s, 2 H) 7.21-7.29 (m, 1 H) 7.29-7.44 (m, 4 H) 7.62 (s, 2 H) 7.88 (d, J = 8.7 Hz, 2 H) 8.04 (d, J = 8.7 Hz, 2 H) 8.56 (bs, 3 H) | | 75.10 | | |
| H- 1r | 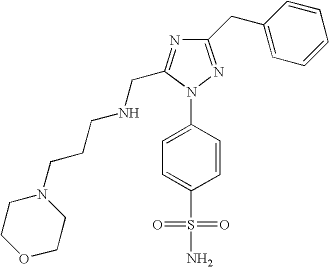 | 4-(3-benzyl-5-{[(3- morpholin-4- ylpropyl)amino]methyl}- 1H-1,2,4-triazol-1- yl)benzenesulfon- amide | 471 | ¹H NMR (400 MHz, DMSO- d₆) 2.11-2.27 (m, 2 H) 2.94- 3.10 (m, 2 H) 3.15-3.25 (m, 4H) 3.37 (d, J = 12.13 Hz, 2 H) 3.78-4.03 (m, 4 H) 4.12 (s, 2 H) 4.51 (s, 2 H) 7.21-7.27 (m, J = 7.07 Hz, 1 H) 7.30-7.40 (m, 4 H) 7.63 (s, 2 H) 7.88 (d, J = 8.7 Hz, 2 H) 8.03 (d, J = 8.7 Hz, 2 H) 9.96 (s, 2 H) 11.61 (s, 1 H) | | 12.10 | | |
| H- 1s | 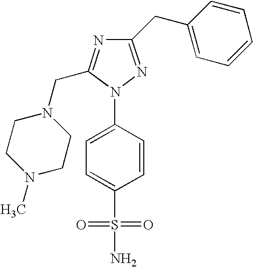 | 4-{3-benzyl-5-[(4- methylpiperazin-1- yl)methyl]-1H-1,2,4- triazol-1- yl}benzenesulfon- amide | 427 | ¹H NMR (300 MHz, DMSO- d₆) 2.75 (s, 3 H) 2.90-3.06 (m, 2 H) 3.07- 3.23 (m, 2 H) 3.23-3.36 (m, 2 H) 3.36-3.55 (m, 2 H) 4.10 (s, 2 H) 4.14 (s, 2 H) 7.20- 7.27 (m, 1 H) 7.28-7.41 (m, 4 H) 7.60 (s, 2 H) 7.92 (d, J = 8.7 Hz, 2 H) 8.03 (d, J = 8.7 Hz, 2 H) 11.42 (s, 1 H) | | 25.70 | | |
| H-1 1t | 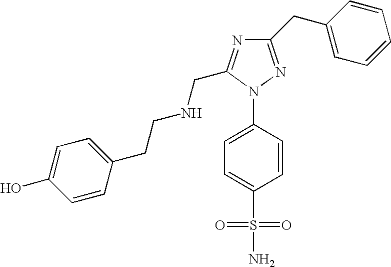 | 4-[3-benzyl-5-({[2-(4- hydroxyphenyl)ethyl] amino]methyl}-1H- 1,2,4-triazol-1- yl]benzenesulfon- amide | 464 | ¹H NMR (300 MHz, DMSO- d₆) 2.80-2.83 (m, 2 H) 3.14- 3.29 (m, 2 H) 4.14 (s, 2 H) 4.56 (s, 2 H) 6.73 (d, J = 8.45 Hz, 2 H) 7.00 (d, J = 8.5 Hz, 2 H) 7.22-7.30 (m, 1 H) 7.31- 7.44 (m, 4 H) 7.62 (s, 2 H) 7.85 (d, J = 8.6 Hz, 2 H) 8.03 (d, J = 8.6 Hz, 2 H) 9.38 (s, 1H) 9.59 (s, 2 H) | | 4.19 | 1360 | |
| H- 1u | 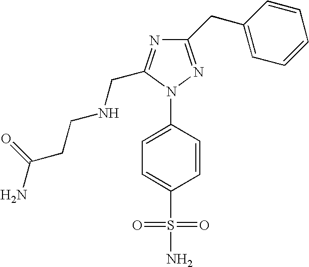 | N-3--({1-[4- (aminosulfonyl)phenyl]- 3-benzyl-1H-1,2,4- triazol-5-yl]methyl}- beta-alaninamide | 415 | ¹H NMR (300 MHz, DMSO- d₆) ppm 2.73 (t, J = 7.4 Hz, 2 H) 3.26 (t, J = 7.4 Hz, 2 H) 4.13 (s, 2 H) 4.55 (s, 2 H) 7.06 (s,1 H) 7.21-7.29 (m, 2 H) 7.30- 7.44 (m, 5 H) 7.62 (s, 2 H)7.85 (d, J = 8.7 Hz, 2 H) 8.03 (d, J = 8.7 Hz, 2 H) | | 14.10 | | |
| H- 1v | 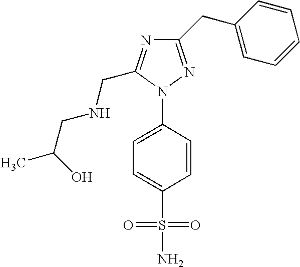 | 4-(3-benzyl-5-{[(2- hydroxypropyl)amino] methyl}-1H-1,2,4- triazol-1- yl)benzenesulfon- amide | 402 | ¹H NMR (300 MHz, DMSO- d₆) 1.10 (d, J = 6.40 Hz, 3 H) 2.86-3.00 (m, 1 H) 3.05- 3.19 (m, 1 H) 3.92-4.06 (m, 1 H) 4.14 (s, 2 H) 4.52 (s, 2 H) 7.22-7.29 (m, 1 H) 7.30-7.42 (m, 4 H) 7.62 (s, 2 H) 7.84 (d, UJ = 8.7 Hz, 2 H) 8.03 (d, J = 8.7 Hz, 2 H) 9.20 (s, 1 H) 9.45 (s, 1 H) | | 7.37 | 4430 | |
| H- 1w | 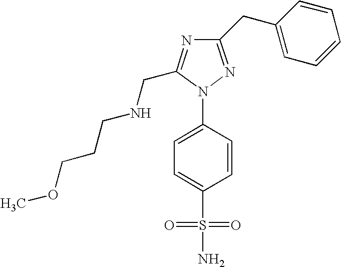 | 4-(3-benzyl-5-{[(3- methoxypropyl)amino] methyl}-1H-1,2,4- triazol-1- yl)benzenesulfon- amide | 416 | ¹H NMR (300 MHz, DMSO- d₆) 1.84-1.96 (m, 2 H) 3.10 (t, J = 7.4 Hz, 2 H) 3.23 (s,3 H) 3.38 (t, J = 7.4 Hz, 2 H) 4.14 (s, 2 H) 4.55 (s, 2 H) 7.22-7.30 (m, 1 H) 7.30- 7.42 (m, 4 H) 7.62 (s, 2 H) 7.86 (d, J = 8.6 Hz, 2 H) 8.03 (d, J = 8.6 Hz, 2 H) 9.46 (s, 2 H) | | 8.43 | | |
| H- 1x | 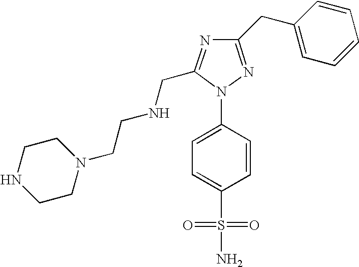 | 4-(3-benzyl-5-{[(2- piperazin-1- ylethyl)amino]methyl}- 1H-1,2,4-triazol-1- yl)benzenesulfon- amide | 456 | ¹H NMR (300 MHz, DMSO- d₆) 2.83-3.57 (m, 12 H) 4.06 (s, 2 H) 4.10 (s, 2 H) 7.20-7.28 (m, 1 H) 7.28- 7.41 (m, J = 7.16 Hz, 4 H) 7.60 (s, 2 H) 7.92 (d, J = 8.6 Hz, 2 H) 8.03 (d, J = 8.6 Hz,2 H) 8.51 (s, 3 H) | | 85.50 | | |
| I-1 | 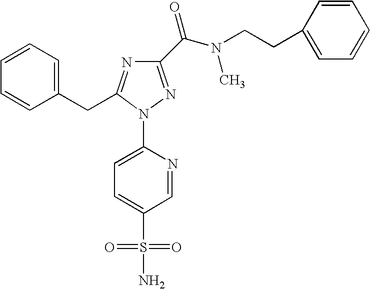 | 1-[5- (aminosulfonyl)pyridin- 2-yl[-5-benzyl-N- methyl-N-(2- phenylethyl)-1H-1,2,4- triazol-3- carboxamide | 477 | ¹H NMR (300 MHz, DMSO- d₆) 2.84-2.94 (m, 2 H) 3.06 (d, J = 9.3 Hz,3 H) 3.62-3.75 (m, 2 H) 4.71 (s, 2 H) 6.99- 7.07 (m, 1 H) 7.10-7.18 (m, 2 H) 7.18-7.35 (m, 7 H) 7.74 (s, 2 H) 7.98- 8.11 (m, 1 H) 8.39-8.46 (m, 1 H) 8.95 (d, J = 1.9 Hz, 1 H) | | 2.46 | 161 | 0.236 |
| I-1a | 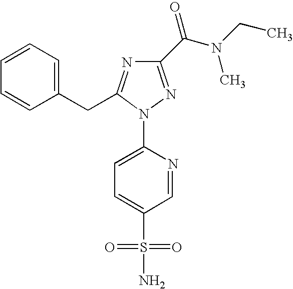 | 1-[5- (aminosulfonyl)pyridin- 2-yl]-5-benzyl-N-ethyl- N-methyl-1H-1,2,4- triazole-3- carboxamide | 401 | ¹H NMR (300 MHz, DMSO- d₆) 4.72 (s, 2 H) 7.19-7.35 (m, 5 H) 7.77 (s,2 H) 8.09 (dd, J = 8.7, 6.2 Hz, 1 H) 8.40- 8.47 (m, 1 H) 8.96 (d,J = 1.7 Hz,1 H) | | 2.50 | 425 | 0.373 |
| I-4 | 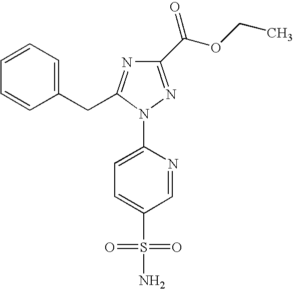 | ethyl 1-[5- (aminosulfonyl)pyridin- 2-yl]-5-benzyl-1H- 1,2,4-triazol-3- carboxylate | 388 | ¹H NMR (300 MHz, DMSO- d₆) 1.35 (t, J = 7.0 Hz, 3 H) 4.40 (q, J = 7.0 Hz, 2 H) 4.72 (s, 2 H) 7.19- 7.35 (m, 5 H) 7.78 (s, 2 H) 8.13 (d, J = 8.7 hz, 1 H) 8.46 (dd, J = 8.7, 2.3 Hz, 1 H) 8.97 (d, J = 2.3 Hz, 1 H) | | 2.58 | 379 | 0.576 |
| I-5 | 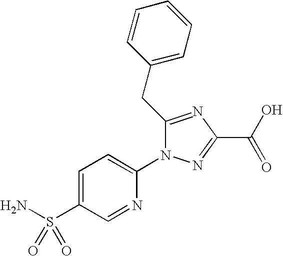 | 1-[5- (aminosulfonyl)pyridin- 2-yl]-5-benzyl-1H- 1,2,4-triazole-3- carboxylic acid | 360 | ¹H NMR (300 MHz, DMSO- d₆) 4.71 (s, 2 H) 7.20-7.33 (m, 5 H) 7.77 (s, 2 H) 8.12 (d, J = 8.7 Hz, 1 H) 8.46 (dd, J = 8.7, 2.4 Hz, 1 H) 8.96 (d, J = 2.4 Hz, 1 H) | | 6.83 | 379 | |
| J-1 | 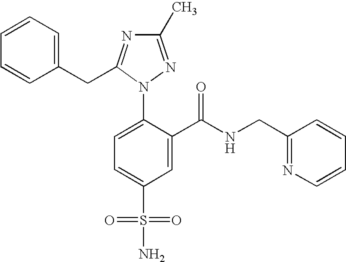 | 5-(aminosulfonyl)-2- (5-benzyl-3-methyl- 1H-1,2,4-triazol-1-yl)- N-(pyridin-2- ylmethyl)benzamide | 463 | ¹H NMR (300 MHz, DMSO- d₆) 2.22 (s, 3 H) 4.01 (s, 2 H) 4.71 (d, J = 5.7 Hz, 2 H) 7.13- 7.19 (m, 2 H) 7.20-7.32 (m, 3 H) 7.68 (s, 1 H) 7.71 (s, 2 H) 7.79 (d, J = 7.8 Hz, 1 H) 7.87 (t, J = 7.8 Hz, 1 H) 8.06 (dd, J = 8.3, 1.9 Hz, 1 H) 8.27 (d, J = 1.9 Hz, 1 H) 8.46 (t, J = 7.8 Hz,1 H) 8.83 (d, J = 5.3 Hz, 1 H) 9.63 (t, J = 5.7 Hz, 1 H) | | 2.84 | 1340 | |
| j-2 | 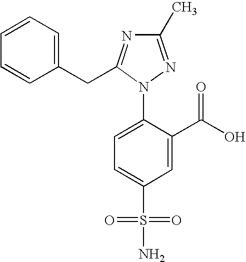 | 5-(aminosulfonyl)-2- (5-benzyl-3-methyl- 1H-1,2,4-triazol-1- yl)benzoic acid | 373 | ¹H NMR (300 MHz, DMSO- d₆) 2.27 (s, 3 H) 3.97 (s, 2 H) 7.09-7.15 (m, 2 H) 7.17-7.30 (m, 3 H) 7.66 (d, J = 8.1 Hz, 1 H) 7.70 (s, 2 H) 8.06 (dd, J = 8.1, 2.2 Hz, 1 H) 8.38 (d, J = 2.2 Hz, 1 H) 13.62 (s, 1 H) | | 3.39 | 4470 | |
| K-1 | 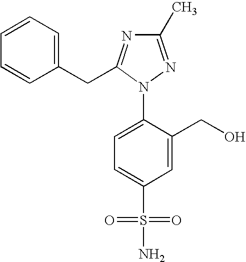 | 4-(5-benzyl-3-methyl- 1H-1,2,4-triazol-1-yl)- 3- (hydroxymethyl)benzene- sulfonamide | 359 | ¹H NMR (300 MHz, DMSO- d₆) 2.31 (s, 3 H) 3.96 (s, 2 H) 4.17 (s, 2 H) 7.022-7.09 (m, 2 H) 7.18-7.30 (m, 3 H) 7.53 (d, J = 8.2 Hz, 1 H) 7.59 (s, 2 H) 7.84 (dd, J = 8.2, 2.0 Hz, 1 H)8.14 (d, J = 2.0 Hz, 1 H) | | 4.22 | | |
| L-1 | 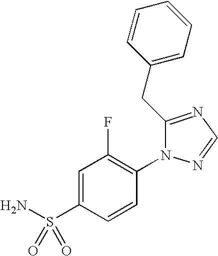 | 4-(5-benzyl-1H-1,2,4- triazol-1-yl)- 3- fluorobenzenesulfon- amide | 333 | ¹H NMR (300 MHz, DMSO- d6) 4.13 (s, 2 H) 7.02-7.09 (m, 2 H) 7.18- 7.28 (m, 3 H) 7.73 (br. s., 2 H) 7.78-7.89 (m, 3 H) 8.22 (s, 1 H) | | | 380 | 0.278 |
| L- 1a | 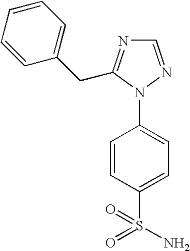 | 4-(5-benzyl-1H-1,2,4- triazol-1- yl)benzenesulfon- amide | 315 | 1H NMR (400 MHz, DMSO- d₆) 8.15 (s, 1 H) 7.94-8.02 (m, 2 H) 7.71- 7.82 (s,2 H), 7.18-7.30 (m, 3 H), 7.11 (d, J = 7.8 Hz, 2 H), 4.28 (s, 2 H). | | | 71.7 | 0.113 |
| M-1 | 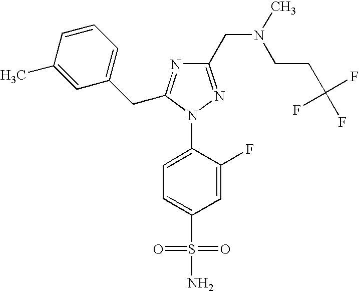 | 3-fluoro-4-(5-(3- methylbenzyl)-3- {[methyl(3,3,3- trifluoropropyl)amino] methyl}-1H-1,2,4- triazol-1- yl)benzenesulfon- amide | 486 | ¹H NMR (300 MHz, DMSO- d6) 2.18-2.24 (m, 3 H) 2.87- 2.92 (m, 3 H), 2.93-3.05 (m, 2 H), 3.41- 3.54 (m, 2 H), 4.11-4.17 (m, 2 H), 4.56- 4.64 (m, 2 H), 6.84-6.90 (m, 2 H), 7.00- 7.06 (m, 1 H), 7.10-7.17 (m, 1 H), 7.75- 7.95 (m, 5 H). | | | 81 | 0.081 |
| M- 1a | 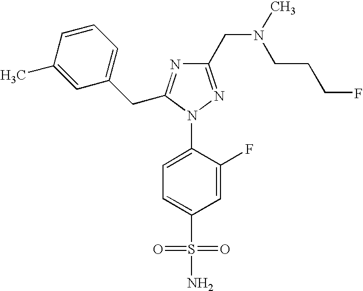 | 3-fluoro-4-[3-{[(3- fluoropropyl)(methyl) amino]methyl}-5-(3- methyl;benzyl)-1H- 1,2,4-triazol-1- yl]benzenesulfon- amide | 450 | ¹H NMR (300 MHz, DMSO- d6) 2.06-2.32 (m,5 H), 2.84- 2.91 (m, 3 H), 3.16-3.38 (m, 2 H), 4.12- 4.16 (m,2 H), 4.43-4.66 (m, 4 H), 6.82- 6.89 (m, 2 H), 7.00-7.06 (m, 1 H), 7.10- 7.17 (m, 1 H), 7.75-7.94 (m, 5 H). | | 1.83 | 486 | 0.15 |
| N-1 | 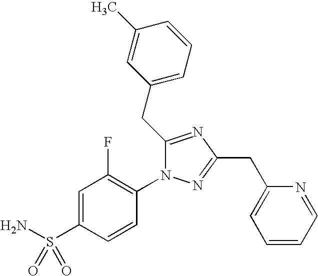 | 3-fluoro-4-[5-(3- methylbenzyl)-3- (pyridin-2-ylmethyl)- 1H-1,2,4-triazol-1- yl]benzenesulfon- amide | 438 | ¹H NMR (300 MHz, DMSO- d₆) 2.22 (s, 3 H) 4.13 (s, 2 H) 8.07 (s, 2 H) 6.75-6.81 (m, 2 H) 6.96-7.13 (m, 2 H) 7.59 (t, J = 7.80 Hz,1 H) 7.82 (q, J = 1.63 Hz, 1 H) 7.84-7.86 (m, 1 H) 8.23 (t, J = 7.25 Hz, 2 H) 8.72 (t, J = 7.82 Hz, 1 H) 9.21 (d, J = 5.65 Hz, 2 H) | | | 101 | 0.444 |
| O-1 | 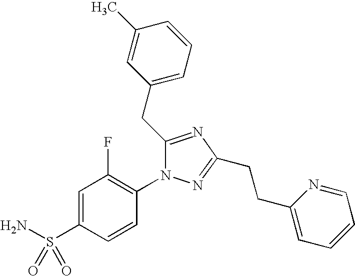 | 3-fluoro-4-[5-(3- methylbenzyl)-3-(2- pyridin-2-ylethyl)-1H- 1,2,4-triazol-1- yl]benzenesulfon- amide | 452 | ¹H NMR (300 MHz, DMSO- d₆) 2.24 (s, 3 H) 3.15-3.32 (m, 4 H) 4.10 (s,2 H) 6.72-6.77 (m, 2 H) 7.01 (d, J = 7.50 Hz, 1 H) 7.10 (t, J = 7.91 Hz, 1 H) 7.26-7.29 (m, 1 H) 7.32 (d, J = 7.72 Hz, 1 H) 7.51 (t, J = 7.80 Hz, 1 H) 7.70-7.85 (m, 3 H) 8.46- 8.51 (m, 1 H) | | | 197 | 0.075 |
| P-1 | 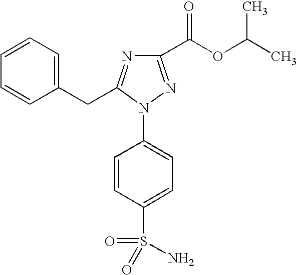 | isopropyl 1-[4- (aminosulfonyl)phenyl]- 5-benzyl-1H-1,2,4- triazole-3-carboxylate | 401 | ¹H NMR (400 MHz, CDCl₃) 1.38 (d, J = 6.3 Hz, 6 H) 4.19 (s, 2 H) 4.97 (s, 2 H) 5.26-5.41 (m, 1 H) 7.02 (d, J = 6.6 Hz, 2 H) 7.12-7.25 (m, 3 H) 7.39 (d, J = 8.6 Hz, 2 H) 7.92 (d, J = 8.6 Hz, 2 H) | 41.0 | 0.15 | | 486 |
| Q-1 | 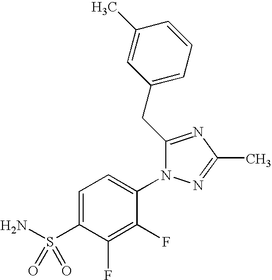 | 2,3-difluoro-4-[3- methyl-5-(3- methylbenzyl)-1H- 1,2,4-triazol-1- yl]benzenesulfon- amide | 379.1 | 1H NMR (300 MHz, MeOH) d ppm 2.24 (s, 3 H) 2.44 (s, 3 H) 4.02 (d, J = 16.50 Hz, 1 H) 4.14 (d, J = 16.50 Hz, 1 H) 6.81-6.88 (m, 2 H) 7.01 (d, J = 7.70 Hz, 1 H) 7.08 (t, J = 7.72 Hz, 1 H) 7.61-7.73 (m,1 H) 7.95- 8.03 (m, 1 H) | 4610.0 | | 0% 69 0.010 nM | 51% @ 5 uM |
| A-1j | 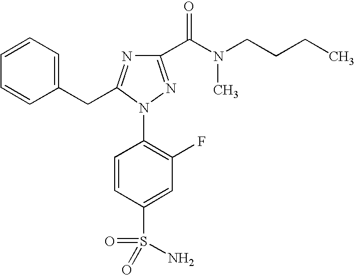 | 1-[4-(aminosulfonyl)-2- fluorophenyl]-5- benzyl-N-butyl-N- methyl-1H-1,2,4- triazole-3- carboxamide | 446 | | 116.0 | 0.24 | | 169 |
| A- 1k | 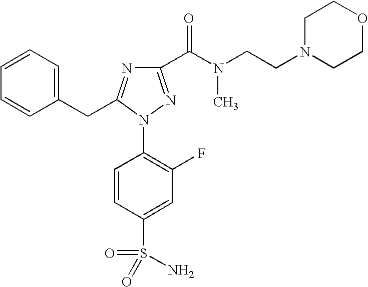 | 1-[4-(aminosulfonyl)-2- fluorophenyl]-5- benzyl-N-methyl-N-(2- morpholin-4-ylethyl)- 1H-1,2,4-triazole-3- carboxamide | 503 | | 23.0 | 0.22 | | 148 |
| I-5b | 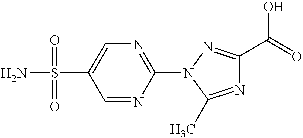 | 1-[5- (aminosulfonyl)pyrimidin- 2-yl]-5-methyl-1H- 1,2,4-triazole-3- carboxylic acid | 283 | 1H NMR (400 MHz, DMSO- d6) ppm 3.93 (s, 3 H) 7.95 (s, 2 H) 9.30 (s, 2 H) 9.67 (s, 1 H) | N D | ND | 57.9 | >1000 |
| A- 1m | 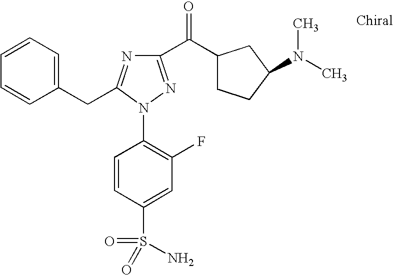 | 4-(5-benzyl-3-{[(3S)-3- (dimethylamino)pyrrolidin- 1-yl]carbonyl}-1H- 1,2,4-triazol-1-yl)-3- fluorobenzenesulfon- amide | 473 | | 21.0 | 0.20 | | 429 |

| Example | | | LRMS | | IC50_CAI | Kd_CAII | IC50_CAII | IC50_CAIV |
|---|---|---|---|---|---|---|---|---|
| number | STRUCTURE | IUPACNAME | m/z (M + H)⁺ | ¹H NMR | (nM) | (nM) | (nM) | (nM) |
| AA-1 | 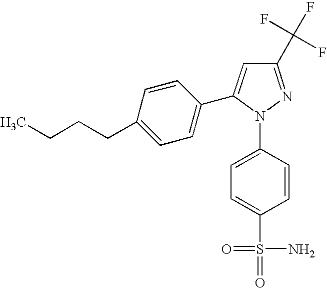 | 4-[5-(4- butylphenyl)-3- (trifluoromethyl)- 1H-pyrazol-1- yl]benzenesulfonamide | 424 | ¹H NMR (400 MHz, DMSO-d6) 0.72 (t, J = 7.33 Hz, 3 H) 1.07- 1.19 (m, 2 H) 1.32- 1.42 (m, 2 H) 2.42 (t, J = 7.70 Hz, 2 H) 7.02- 7.10 (m, 5 H) 7.35- 7.40 (m, 4 H) 7.70 (d, J = 8.59 Hz, 2 H) | | 146 | | |
| AA- 1a | 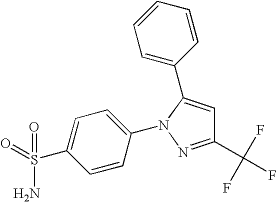 | 4-[5-phenyl-3- (trifluoromethyl)- 1H-pyrazol-1- yl]benzenesulfonamide | 388 | ¹H NMR (400 MHz, DMSO-d6) 7.07 (s, 1 H) 7.11-7.17 (m, 2 H) 7.20-7.26 (m, 3 H) 7.34 (s, 2 H) 7.38 (d, J = 8.59 Hz, 2 H) 7.68 (d, J = 8.59 Hz, 2 H) | | 46.7 | | 1000 |
| AA- 1b | 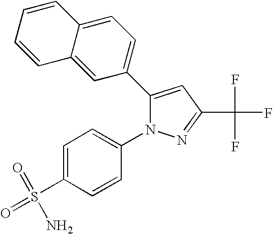 | 4-[5-(2-naphthyl)- 3-(trifluoromethyl)- 1H-pyrazol-1- yl]benzenesulfonamide | 418 | ¹H NMR (400 MHz, DMSO-d6) 7.27 (dd, J = 8.59, 1.77 Hz, 1 H) 7.34 (s, 1 H) 7.48 (s, 2 H) 7.54-7.59 (m, 4 H) 7.83 (d, J = 8.59 Hz, 2 H) 7.88-7.94 (m, 3 H) 8.03 (s, 1 H) | | 73.2 | | |
| AA- 1c | 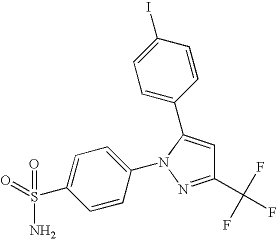 | 4-[5-(4- iodophenyl)-3- (trifluoromethyl)- 1H-pyrazol-1- yl]benzenesulfonamide | 494 | ¹H NMR (400 MHz, DMSO-d6) 6.97 (d, J = 8.34 Hz, 2 H) 7.15 (s, 1 H) 7.38 (s, 2 H) 7.42 (d, J = 8.59 Hz, 2 H) 7.66 (d, J = 8.34 Hz, 2 H) 7.75 (d, J = 8.59 Hz, 2 H) | | 88.6 | | |
| AA- 1d | 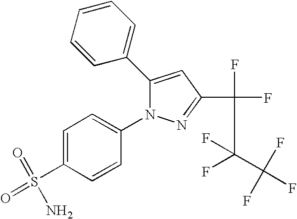 | 4-[3- (heptafluoropropyl)- 5-phenyl-1H- pyrazol-1-yl] benzenesulfonamide | 488 | ¹H NMR (400 MHz, DMSO-d6) 7.45 (s, 1 H) 7.49-7.54 (m, 2 H) 7.57-7.63 (m, 3 H) 7.68-7.74 (m, 4 H) 8.08 (d, J = 8.59 Hz, 2 H) | | 163 | | |
| AA- 1e | 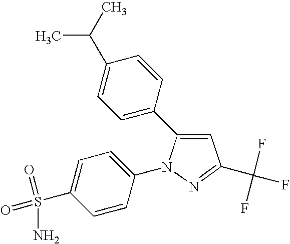 | 4-[5-(4- isopropylphenyl)- 3-(trifluoromethyl)- 1H-pyrazol-1- yl]benzenesulfonamide | 410 | ¹H NMR (400 MHz, DMSO-d6) 1.10 (d, J = 6.82 Hz, 6 H) 2.77- 2.85 (m, 1 H) 7.12 (s, 1 H) 7.15 (d, J = 8.10 Hz, 2 H) 7.20 (d, J = 8.10 Hz, 2 H) 7.44 (s, 2 H) 7.47 (d, J = 8.59 Hz, 2 H) 7.79 (d, J = 8.59 Hz, 2 H) | | 309 | | |
| AA- 1f | 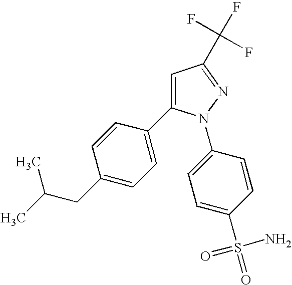 | 4-[5-(4- isobutylphenyl)-3- (trifluoromethyl)- 1H-pyrazol-1- yl]benzenesulfonamide | 424 | ¹H NMR (400 MHz, DMSO-d6) 0.78 (d, J = 6.57 Hz, 6 H) 1.73- 1.81 (m, 1 H) 2.40 (d, J = 7.07 Hz, 2 H) 7.11- 7.16 (m, 5 H) 7.44- 7.50 (m, 4 H) 7.60 (d, J = 8.59 Hz, 2 H) | | 1000 | | |
| AA- 1g | 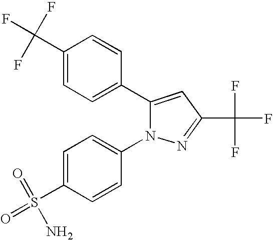 | 4-{3- (trifluoromethyl)-5- [4-(trifluoromethyl) phenyl]-1H- pyrazol-1- yl}benzenesulfonamide | 435 | ¹H NMR (400 MHz, DMSO-d6) 7.47 (s, 1 H) 7.58-7.69 (m, 6 H) 7.88 (d, J = 8.08 Hz, 2 H) 7.98 (d, J = 8.59 Hz, 2 H) | | 43.4 | | |
| BB-1 | 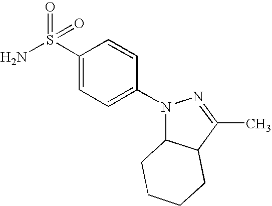 | 4-(3-methyl- 3a,4,5,6,7,7a- hexahydro-1H- indazol-1-yl) benzenesulfonamide | 294 | ¹H NMR (400 MHz, DMSO-d6) 0.52- 0.68 (m, 1 H) 0.81- 0.98 (m, 1 H) 1.27- 1.38 (m, 2 H) 1.43- 1.57 (m, 1 H) 1.68- 1.84 (m, 5 H) 2.88- 2.94 (m, 1 H) 2.95 (d, J = 5.31 Hz, 1 H) 4.13- 4.21 (m, 1 H) 6.81 (s, 2 H) 6.86 (d, J = 8.84 Hz, 2 H) 7.39 (d, J = 9.09 Hz, 2 H) | | 67.2 | | |
| CC- 1 | 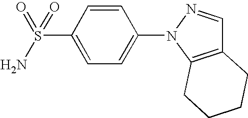 | 4-(4,5,6,7- tetrahydro-1H- indazol-1-yl) benzenesulfonamide | 278 | ¹H NMR (400 MHz, DMSO-d6) 1.52- 1.82 (m, 4 H) 2.34- 2.38 (m, 2 H) 2.64 (t, J = 5.58 Hz, 2 H) 7.29 (br. s., 2 H) 7.37 (s, 1 H) 7.59 (d, J = 8.84 Hz, 2 H) 7.75 (d, J = 8.59 Hz, 2 H) | | 15 | | |
| CC- 1a | 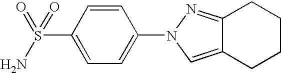 | 4-(4,5,6,7- tetrahydro-2H- indazol-2-yl) benzenesulfonamide | 278 | ¹H NMR (400 MHz, DMSO-d6) 1.43- 1.59 (m, 4 H) 2.34 (t, J = 6.06 Hz, 2 H) 2.43 (t, J = 6.32 Hz, 2 H) 7.13 (br. s., 2 H) 7.63 (d, J = 8.70 Hz, 2 H) 7.70 (d, J = 8.70 Hz, 2 H) 8.04 (s, 1 H) | | 19.6 | | |
| DD 1 | 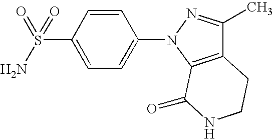 | 4-(3-methyl-7-oxo- 4,5,6,7-tetrahydro- 1H-pyrazolo[3,4- c]pyridin-1-yl) benzenesulfonamide | 307 | ¹H NMR (400 MHz, DMSO-d6) 2.25 (s, 3 H) 2.70 (t, J = 6.69 Hz, 2 H) 3.64-3.75 (m, 2 H) 7.45 (br. s., 2 H) 7.74 (d, J = 8.59 Hz, 2 H) 7.86 (d, J = 8.59 Hz, 2 H) 7.90 (br. s., 1 H) | | 143 | | |
| EE-1 | 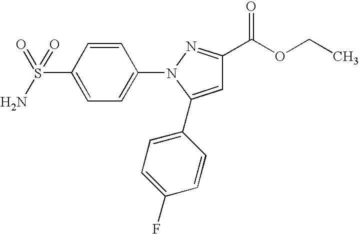 | ethyl 1-[4- (aminosulfonyl) phenyl]-5-(4- fluorophenyl)- 1H-pyrazole-3- carboxylate | 390 | ¹H NMR (400 MHz, DMSO-d6) 1.19 (t, J = 7.07 Hz, 3 H) 4.22 (q, J = 7.07 Hz, 2 H) 7.03 (s, 1 H) 7.13 (td, J = 8.00, 2.30 Hz, 2 H) 7.23 (td, J = 6.00, 2.50 Hz, 2 H) 7.36-7.42 (m, 4 H) 7.75 (d, J = 8.59 Hz, 2 H) | | 130 | | |
| EE- 1a | 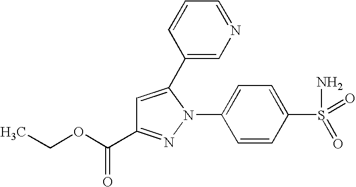 | ethyl 1-[4- (aminosulfonyl) phenyl]-5-pyridin-3- yl-1H-pyrazole-3- carboxylate | 373 | ¹H NMR (300 MHz, DMSO-d₆) 1.35 (t, J = 7.1 Hz, 3 H) 4.38 (q, J = 7.1 Hz, 2 H) 7.40 (s, 1 H) 7.53- 7.68 (m, 5 H) 7.88- 7.94 (m, 3 H) 8.68- 8.74 (m, 2 H | | 270 | | |
| EE- 1b | 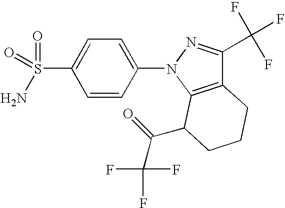 | 4-[7- (trifluoroacetyl)-3- (trifluoromethyl)- 4,5,6,7- tetrahydro-1H- indazol-1- yl]benzenesulfonamide | 442 | ¹H NMR (400 MHz, DMSO-d6) 1.58- 1.70 (m, 1 H) 1.74- 1.83 (m, 1 H) 1.85- 1.94 (m, 1 H) 2.26- 2.36 (m, 1 H) 2.61 (t, J = 5.68 Hz, 2 H) 5.19 (t, J = 6.19 Hz, 1 H) 7.49 (s, 2 H) 7.69 (d, J = 8.59 Hz, 2 H) 7.88 (d, J = 8.59 Hz, 2 H) | | 6.3 | | |
| EE- 1c | 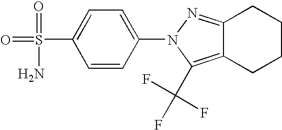 | 4-[3- (trifluoromethyl)- 4,5,6,7-tetrahydro- 2H-indazol-2-yl] benzenesulfonamide | 346 | ¹H NMR (400 MHz, DMSO-d6) 1.66- 1.75 (m, 4 H) 2.58- 2.66 (m, 4 H) 7.48 (s, 2 H) 7.80 (d, J = 8.59 Hz, 2 H) 7.90 (d, J = 8.59 Hz, 2 H) | | 2.22 | 2.7 | |
| EE- 1d | 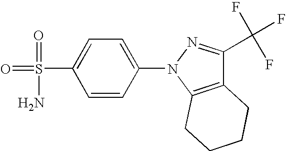 | 4-[3- (trifluoromethyl)- 4,5,6,7-tetrahydro- 1H-indazol-1-yl] benzenesulfonamide | 346 | ¹H NMR (400 MHz, DMSO-d6) 1.67- 1.73 (m, 4 H) 2.53- 2.57 (m, 2 H) 2.73- 2.78 (m, 2 H) 7.46 (s, 2 H) 7.76 (d, J = 8.59 Hz, 2 H) 7.91 (d, J = 8.59 Hz, 2 H) | | 3.65 | 4.1 | 1000 |
| EE- 1e | 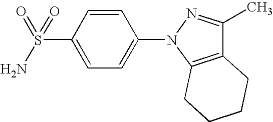 | 4-(3-methyl- 4,5,6,7-tetrahydro- 1H-indazol-1-yl) benzenesulfonamide | 292 | ¹H NMR (400 MHz, DMSO-d6) 1.67- 1.75 (m, 4 H) 2.12 (s, 3 H) 2.35-2.43 (m, 2 H) 2.74-2.81 (m, 2 H) 7.37 (br. s., 2 H) 7.70 (d, J = 8.59 Hz, 2 H) 7.66 (d, J = 8.59 Hz, 2 H) | | 15.9 | | |
| EE- 1f | 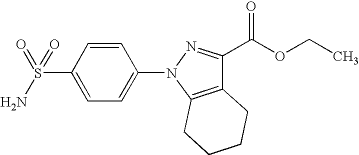 | ethyl 1-[4- (aminosulfonyl) phenyl]- 4,5,6,7- tetrahydro-1H- indazole-3- carboxylate | 350 | ¹H NMR (400 MHz, DMSO-d6) 1.31 (t, J = 7.07 Hz, 3 H) 1.72- 1.79 (m, 4 H) 2.70- 2.75 (m, 2 H) 2.77- 2.83 (m, 2 H) 4.31 (q, J = 7.07 Hz, 2 H) 7.50 (br. s., 2 H) 7.62 (d, J = 8.84 Hz, 2 H) 7.98 (d, J = 8.64 Hz, 2 H) | | 3.17 | | |
| EE- 1g | 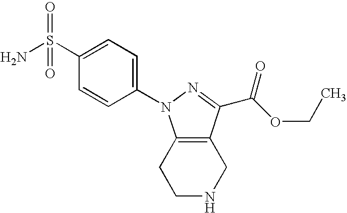 | ethyl 1-[4- (aminosulfonyl) phenyl]-4,5,6,7- tetrahydro-1H- pyrazolo[4,3- c]pyridine-3- carboxylate | 351 | ¹H NMR (300 MHz, DMSO-d₆) 1.35 (t, J = 7.2 Hz, 3 H) 3.20 (t, J = 5.7 Hz, 2 H) 3.41 (t, J = 5.7 Hz, 2 H) 4.32- 4.42 (m, 4 H) 7.59 (s, 2 H) 7.87 (d, J = 8.7 Hz, 2 H) 8.04 (d, J = 8.7 Hz, 2 H) 9.82 (s, 1 H) | | 4.93 | | 1000 |
| FF-1 | 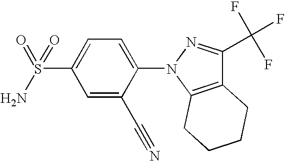 | 3-cyano-4-[3- (trifluoromethyl)- 4,5,6,7- tetrahydro-1H- indazol-1- yl]benzenesulfonamide | 371 | ¹H NMR (400 MHz, DMSO-d₆) 1.84- 2.00 (m, 4 H) 3.16- 3.27 (m, 4 H) 7.44 (s, 2 H) 8.02 (s, 2 H) 8.69 (s, 1 H) | | 36.9 | | |
| FF- 1a | 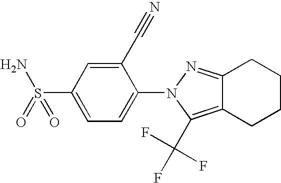 | 3-cyano-4-[3- (trifluoromethyl)- 4,5,6,7- tetrahydro-2H- indazol-2- yl]benzenesulfonamide | 371 | ¹H NMR (400 MHz, DMSO-d6) 1.97- 2.06 (m, 2 H) 2.08- 2.17 (m, 2 H) 3.13 (t, J = 5.56 Hz, 2 H) 3.46 (t, J = 6.19 Hz, 2 H) 7.54 (s, 2 H) 8.11- 8.22 (m, 2 H) 8.82 (s, 1 H) | | 119 | | |
| GG- 1 | 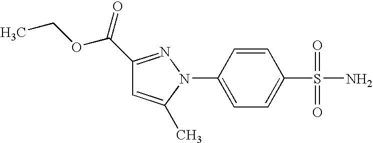 | ethyl 1-[4- (aminosulfonyl) phenyl]- 5-methyl-1H- pyrazole-3- carboxylate | 310 | ¹H NMR (400 MHz, DMSO-d6) 1.30 (t, J = 7.07 Hz, 3 H) 2.41 (s, 3 H) 4.31 (q, J = 7.07 Hz, 2 H) 8.82 (s, 1 H) 7.54 (br. s., 2 H) 7.82 (d, J = 8.59 Hz, 2 H) 8.00 (d, J = 8.59 Hz, 2 H) | | 3.05 | | 5000 |
| GG- 1a | 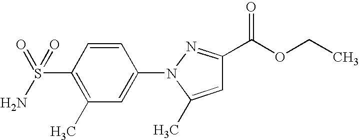 | ethyl 1-[4- (aminosulfonyl)-3- methylphenyl]- 5-methyl-1H- pyrazole-3- carboxylate | 324 | ¹H NMR (400 MHz, ACETONITRILE-d3) 1.26 (t, J = 7.07 Hz, 3 H) 2.31 (s, 3 H) 2.62 (s, 3 H) 4.25 (q, J = 7.24 Hz, 2 H) 6.64 (s, 1 H) 7.41 (dd, J = 8.59, 2.02 Hz, 1 H) 7.46 (d, J = 2.02 Hz, 1 H) 7.98 (d, J = 8.34 Hz, 1 H) | | 6.22 | | |
| GG- 1b | 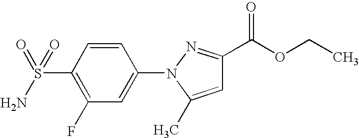 | ethyl 1-[4- (aminosulfonyl)-3- fluorophenyl]- 5-methyl-1H- pyrazole-3- carboxylate | 328 | ¹H NMR (400 MHz, DMSO-d6) 1.36 (t, J = 7.07 Hz, 3 H) 2.49 (s, 3 H) 4.37 (q, J = 7.07 Hz, 2 H) 6.88 (s, 1 H) 7.70 (dd, J = 8.59, 2.02 Hz, 1 H) 7.88 (dd, J = 10.88, 2.02 Hz, 1 H) 7.90 (s, 2 H) 8.02 (t,J = 8.21 Hz, 1 H) | | 2.71 | | |
| GG- 1c | 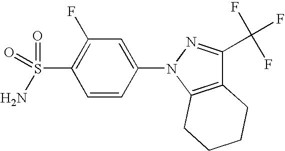 | 2-fluoro-4-[3- (trifluoromethyl)- 4,5,6,7-tetrahydro- 1H-indazol-1-yl] benzenesulfonamide | 364 | ¹H NMR (400 MHz, DMSO-d6) 1.66- 1.72 (m, 4 H) 2.51- 2.68 (m, 2 H) 2.75- 2.81 (m, 2 H) 7.56 (dd, J = 8.59, 2.02 Hz, 1 H) 7.67 (dd, J = 10.86, 2.02 Hz, 1 H) 7.74 (s, 2 H) 7.87 (t, J = 8.34 Hz, 1 H) | | 3.36 | | |
| GG- 1d | 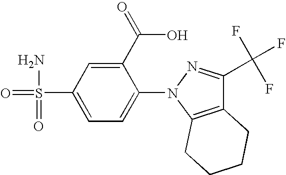 | 5-(aminosulfonyl)- 2-[3- (trifluoromethyl)- 4,5,6,7-tetrahydro- 1H-indazol-1-yl] benzoic acid | 389 (M)⁺ | ¹H NMR (400 MHz, DMSO-d6) 1.57- 1.63 (m, 4 H) 2.39- 2.47 (m, 4 H) 7.38 (s, 1 H) 7.47 (s, 2 H) 7.60 (d, J = 8.34 Hz, 1 H) 7.78 (br. s., 1 H) 7.85 (dd, J = 8.50, 2.00 Hz, 1 H) 7.88 (d, J = 2.02 Hz, 1 H) | | 7.52 | | |
| GG- 1e | 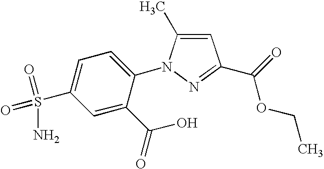 | 5-(aminosulfonyl)- 2-[3- (ethoxycarbonyl)- 5-methyl-1H- pyrazol-1-yl] benzoic acid | 353.0 (M)⁺ | ¹H NMR (400 MHz, DMSO-d6) 1.34 (t, J = 7.07 Hz, 3 H) 2.25 (s, 3 H) 4.33 (q, J = 7.07 Hz, 2 H) 6.77 (s, 1 H) 7.57 (s, 1 H) 7.69 (s, 2 H) 7.78 (d, J = 8.34 Hz, 1 H) 8.01 (s, 1 H) 8.08 (dd, J = 8.20, 2.20 Hz, 1 H) 8.11 (d, J = 2.27 Hz, 1 H) | | 10.5 | | |
| HH- 1 | 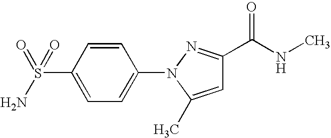 | 1-[4- (aminosulfonyl) phenyl]- N,5-dimethyl-1H- pyrazole-3- carboxamide | 295 | ¹H NMR (400 MHz, DMSO-d6) 2.41 (s, 3 H) 2.75 (d, J = 4.55 Hz, 3 H) 6.68 (s, 1 H) 7.52 (s, 2 H) 7.83 (d, J = 8.59 Hz, 2 H) 7.98 (d, J = 8.59 Hz, 2 H) 8.19 (d, J = 4.80 Hz, 1 H) | | 11 | | |
| HH- 1a | 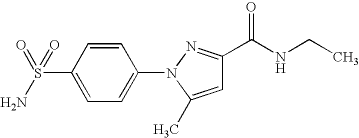 | 1-[4- (aminosulfonyl) phenyl]- N-ethyl-5-methyl- 1H-pyrazole-3- carboxamide | 309 | ¹H NMR (400 MHz, DMSO-d6) 0.89 (t, J = 7.20 Hz, 3 H) 2.20 (s, 3 H) 3.01-3.10 (m, 2 H) 6.47 (s, 1 H) 7.32 (s, 2 H) 7.63 (d, J = 8.59 Hz, 2 H) 7.78 (d, J = 8.84 Hz, 2 H) 8.03 (t, J = 5.81 Hz, 1 H) | | 9.09 | | |
| HH- 1b | 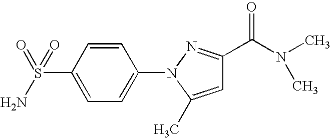 | 1-[4- (aminosulfonyl) phenyl]- N,N,5-trimethyl- 1H-pyrazole-3- carboxamide | 309 | ¹H NMR (400 MHz, DMSO-d6) 2.32 (s, 3 H) 2.89 (s, 3 H) 3.17 (s, 3 H) 6.51 (s, 1 H) 7.42 (s, 2 H) 7.71 (d, J = 8.59 Hz, 2 H) 7.88 (d, J = 8.59 Hz, 2 H) | | 5.01 | | |
| HH- 1c | 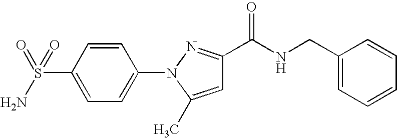 | 1-[4- (aminosulfonyl) phenyl]- N-benzyl-5- methyl-1H- pyrazole-3- carboxamide | 371 | ¹H NMR (400 MHz, DMSO-d6) 2.37 (s, 3 H) 4.39 (d, J = 6.32 Hz, 2 H) 6.68 (s, 1 H) 7.18-7.22 (m, 1 H) 7.24-7.30 (m, 4 H) 7.47 (br. s., 2 H) 7.80 (d, J = 8.59 Hz, 2 H) 7.94 (d, J = 8.59 Hz, 2 H) 8.76 (t, J = 6.32 Hz, 1 H) | | 10.1 | | |
| HH- 1d | 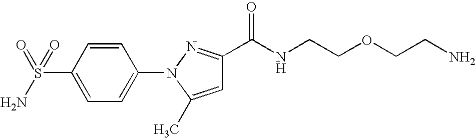 | N-[2-(2- aminoethoxy)ethyl]- 1-[4- (aminosulfonyl) phenyl]- 5-methyl-1H- pyrazole-3- carboxamide | 368.1 | ¹H NMR (400 MHz, DMSO-d6) 2.29 (s, 3 H) 2.83-2.90 (m, 2 H) 3.33 (q, J = 5.56 Hz, 2 H) 3.42-3.52 (m, 4 H) 6.58 (s, 1 H) 7.42 (s, 2 H) 7.65 (br. s., 2 H) 7.71 (d, J = 8.59 Hz, 2 H) 7.87 (d, J = 8.59 Hz, 2 H) 8.13 (t, J = 5.81 Hz, 1 H) | | 9.66 | | |
| II-1 | 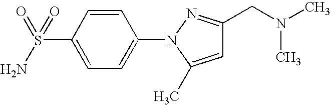 | 4-{3- [(dimethylamino) methyl]- 5-methyl-1H- pyrazol-1- yl}benzenesulfonamide | 296 | ¹H NMR (400 MHz, MeOD) 2.46 (s, 3 H) 2.96 (s, 6 H) 4.38 (s, 2 H) 6.54 (s, 1 H) 7.76 (d, J = 8.59 Hz, 2 H) 8.09 (d, J = 8.34 Hz, 2 H) | | 60.4 | | |
| II-1a | 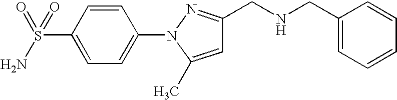 | 4-{3- [(benzylamino) methyl]- 5-methyl-1H- pyrazol-1- yl}benzenesulfonamide | 357 | ¹H NMR (400 MHz, MeOD) 2.46 (s, 3 H) 4.30 (s, 2 H) 4.33 (s, 2 H) 6.48 (s, 1 H) 7.47-7.50 (m, 2 H) 7.52-7.56 (m, 3 H) 7.78 (d, J = 8.59 Hz, 2 H) 8.09 (d, J = 8.59 Hz, 2 H) | | 32.6 | | |
| II-1b | 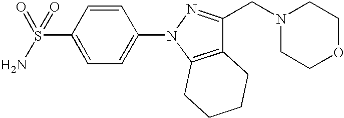 | 4-[3-(morpholin-4- ylmethyl)-4,5,6,7- tetrahydro-1H- indazol-1-yl] benzenesulfonamide | 377 | ¹H NMR (400 MHz, DMSO-d6) 1.62- 1.73 (m, 4 H) 2.51- 2.58 (m, 2 H) 2.71- 2.77 (m, 2 H) 3.04- 3.16 (m, 2 H) 3.23- 3.32 (m, 2 H) 3.38 (d, J = 11.12 Hz, 2 H) 3.71 (t, J = 12.00 Hz, 2 H) 3.88 (d, J = 11.62 Hz, 2 H) 4.19-4.27 (m, 2 H) 7.42 (s, 2 H) 7.73 (d, J = 6.59 Hz, 2 H) 7.88 (d, J = 8.84 Hz, 2 H) 10.89 (br.s., 1 H) | | 11.6 | | 5000 |
| II-1c | 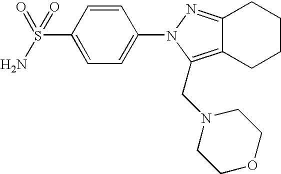 | 4-[3-(morpholin-4- ylmethyl)-4,5,6,7- tetrahydro-2H- indazol-2-yl] benzenesulfonamide | 377 | ¹H NMR (400 MHz, DMSO-d6) 1.81- 2.00 (m, 4 H) 2.79 (t, J = 6.06 Hz, 2 H) 2.85- 2.90 (m, 2 H) 2.96- 3.06 (m, 2 H) 3.20- 3.32 (m, 2 H) 3.88- 3.98 (m, 4 H) 4.59 (s, 2 H) 7.66 (br. s., 2 H) 7.92 (d, J = 8.34 Hz, 2 H) 8.08 (d, J = 8.59 Hz, 2 H) 11.45 (br. s., 1 H) | | 57.9 | | 5000 |
| JJ-1 | 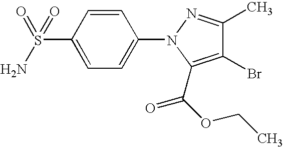 | ethyl 1-[4- (aminosulfonyl) phenyl]- 4-bromo-3- methyl-1H- pyrazole-5- carboxylate | 388 | ¹H NMR (400 MHz, DMSO-d6) 1.34 (t, J = 7.07 Hz, 3 H) 2.40 (s, 3 H) 4.36 (q, J = 7.07 Hz, 2 H) 7.60 (s, 2 H) 7.86 (d, J = 6.59 Hz, 2 H) 8.03 (d, J = 8.59 Hz, 2 H) | | 5.02 | | |
| KK-1 | 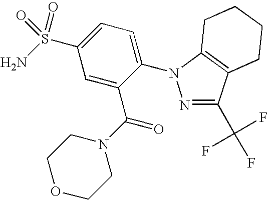 | 3-(morpholin-4- ylcarbonyl)-4-[3- (trifluoromethyl)- 4,5,6,7-tetrahydro- 1H-indazol-1-yl] benzenesulfonamide | 459.0 | ¹H NMR (400 MHz, MeOD) 1.62-1.88 (m, 4 H) 2.47-2.73 (m, 4 H) 3.16-3.67 (m, 8 H) 7.54 (d, J = 8.34 Hz, 1 H) 7.87 (d, J = 2.02 Hz, 1 H) 8.01 (dd, J = 8.34, 2.02 Hz, 1 H) | | 22 | | |
| KK- 1a | 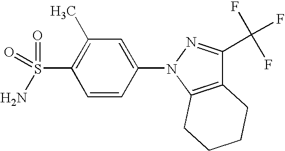 | 2-methyl-4-[3- (trifluoromethyl)- 4,5,6,7-tetrahydro- 1H-indazol-1-yl] benzenesulfonamide | 360 | ¹H NMR (400 MHz, MeOD) 2.54-2.58 (m, 2 H) 2.62-2.64 (m, 2 H) 2.69-2.72 (m, 2 H) 2.75-2.77 (m, 2 H) 2.89 (s, 3 H) 7.42 (dd, J = 8.46, 1.69 Hz, 1 H) 7.48 (d, J = 2.02 Hz, 1 H) 8.00 (d, J = 8.59 Hz, 1 H) | | 8.65 | | |
| KK- 1c | 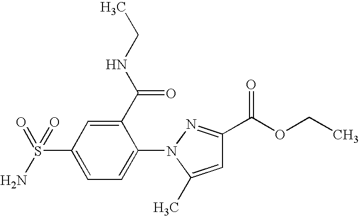 | ethyl 1-{4- (aminosulfonyl)-2- [(ethylamino) carbonyl]phenyl}- 5-methyl-1H- pyrazole-3- carboxylate | 381.0 | ¹H NMR (400 MHz, MeOD) 0.95 (t, J = 7.20 Hz, 3 H) 1.26 (t, J = 7.07 Hz, 2 H) 2.16 (s, 3 H) 3.12 (q, J = 7.33 Hz, 2 H) 4.25 (q, J = 7.07 Hz, 2 H) 6.64 (s, 1 H) 7.59 (d, J = 8.64 Hz, 1 H) 8.03- 8.07 (m, 2 H) | | 39.6 | | |
| KK- 1d | 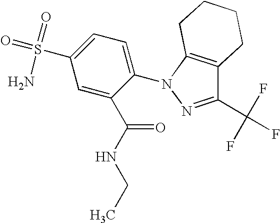 | 5-(aminosulfonyl)- N-ethyl-2-[3- (trifluoromethyl)- 4,5,6,7- tetrahydro-1H- indazol-1- yl]benzamide | 417 | ¹H NMR (400 MHz, ACETONITRILE-d3) 0.91 (t, J = 7.20 Hz, 3 H) 2.02-2.12 (m, 4 H) 2.41-2.47 (m, 2 H) 2.55-2.58 (m, 2 H) 3.05-3.13 (m, 2 H) 7.51 (d, J = 8.34 Hz, 1 H) 7.96 (dd, J = 8.34, 2.27 Hz, 1 H) 8.03 (d, J = 2.02 Hz, 1 H) | | 4.95 | | |
| KK- 1e | 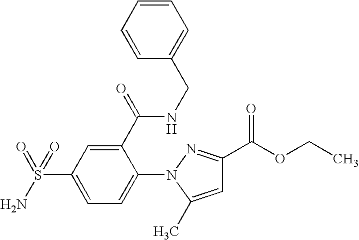 | ethyl 1-{4- (aminosulfonyl)-2- [(benzylamino) carbonyl]phenyl}-5-methyl-1H- pyrazole-3- carboxylate | 443.0 | ¹H NMR (400 MHz, DMSO-d6) 1.36 (t, J = 7.07 Hz, 3 H) 2.24 (s, 3 H) 4.33-4.41 (m, 4 H) 6.77 (s, 1 H) 7.20-7.24 (m, 2 H) 7.28-7.44 (m, 3 H) 7.73 (s, 2 H) 7.83 (d, J = 8.84 Hz, 1 H) 8.09- 8.14 (m, 2 H) 9.21 (t, J = 5.81 Hz, 1 H) | | 5.21 | | |
| KK- 1f | 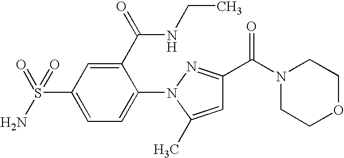 | 5-(aminosulfonyl)- N-ethyl-2-[5- methyl-3- (morpholin-4- ylcarbonyl)- 1H-pyrazol-1- yl]benzamide | 422 | ¹H NMR (400 MHz, DMSO-d6) 0.76 (t, J = 7.20 Hz, 3 H) 2.06 (s, 3 H) 2.82-2.90 (m, 2 H) 3.32-3.45 (m, 6 H) 3.69-3.75 (m, 2 H) 6.36 (s, 1 H) 7.42 (s, 2 H) 7.58 (d, J = 8.34 Hz, 1 H) 7.78 (d, J = 2.02 Hz, 1 H) 7.81 (dd, J = 8.25, 2.00 Hz, 1 H) 8.21 (t, J = 5.43 Hz, 1 H) | | 16.8 | | |
| KK- 1g | 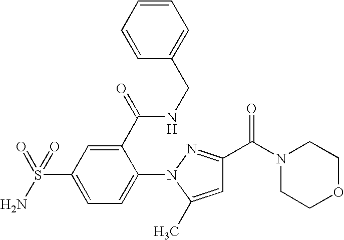 | 5-(aminosulfonyl)- N-benzyl-2-[5- methyl-3- (morpholin-4- ylcarbonyl)- 1H-pyrazol-1- yl]benzamide | 484 | ¹H NMR (400 MHz, DMSO-d6) 2.18 (s, 3 H) 3.33-3.45 (m, 5 H) 3.72-3.77 (m, 2 H) 4.21 (d, J = 6.06 Hz, 2 H) 6.50 (s, 1 H) 7.08 (d, J = 6.82 Hz, 2 H) 7.15-7.24 (m, 3 H) 7.56 (s, 2 H) 7.72 (d, J = 9.09 Hz, 1 H) 7.93-7.96 (m, 2 H) 8.97 (t, J = 5.94 Hz, 1 H) | | 13.1 | | |
| KK- 1h | 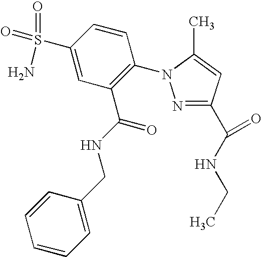 | 1-{4- (aminosulfonyl)-2- [(benzylamino) carbonyl]phenyl}- N-ethyl-5-methyl- 1H-pyrazole-3- carboxamide | 442 | ¹H NMR (400 MHz, DMSO-d6) 1.12 (t, J = 7.20 Hz, 3 H) 2.20 (s, 3 H) 3.23-3.35 (m, 2 H) 4.36 (d, J = 5.81 Hz, 2 H) 6.62 (s, 1 H) 7.19 (d, J = 6.82 Hz, 2 H) 7.25- 7.35 (m, 3 H) 7.69 (s, 2 H) 7.79 (d, J = 8.34 Hz, 1 H) 7.99 (t, J = 5.61 Hz, 1 H) 8.06 (dd, J = 6.34, 2.02 Hz, 1 H)9.03 (t, J = 5.94 Hz, 1 H) | | 10 | | 2190 |
| KK- 1i | 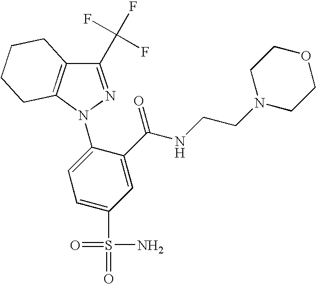 | 5-(aminosulfonyl)- N-(2-morpholin-4- ylethyl)-2-[3- (trifluoromethyl)- 4,5,6,7-tetrahydro- 1H-indazol-1-yl] benzamide | 502 | ¹H NMR (400 MHz, DMSO-d6) 1.81- 1.91 (m, 4 H) 2.69- 2.75 (m, 4 H) 3.12- 3.27 (m, 4 H) 3.63 (d, J = 12.13 Hz, 2 H) 3.63 (q, J = 8.57 Hz, 2 H) 3.90 (t, J = 11.75 Hz, 2 H) 4.07 (d, J = 10.61 Hz, 2 H) 7.77 (s, 2 H) 7.93 (d, J = 8.34 Hz, 1 H) 8.15 (dd, J = 8.34, 2.27 Hz, 1H) 9.04 (t, J = 5.66 Hz, 1 H) 11.20 (br. s., 1 H) | | 33.9 | | 1000 |
| KK- 1j | 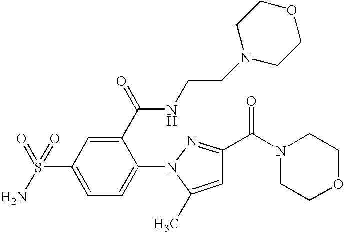 | 5-(aminosulfonyl)- 2-[5-methyl-3- (morpholin-4- ylcarbonyl)-1H- pyrazol-1-yl]-N-(2- morpholin-4- ylethyl) benzamide | 507 | ¹H NMR (400 MHz, MeOD) anisotropism 2.24 (s, 3 H) 3.00- 3.16 (m, 4 H) 3.40- 3.82 (m, 14 H) 3.96 (d, J = 12.63 Hz, 2 H) 6.48 (s, 1 H) 7.64 (d, J = 8.34 Hz, 1 H) 8.08 (d, J = 7.07 Hz, 1 H) 8.17 (d, J = 1.77 Hz, 1 H) | | 173 | | |
| KK- 1k | 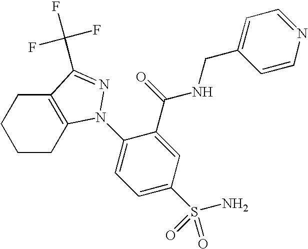 | 5-(aminosulfonyl)- N-(pyridin-4- ylmethyl)-2-[3- (trifluoromethyl)- 4,5,6,7-tetrahydro- 1H-indazol-1-yl] benzamide | 480 | ¹H NMR (400 MHz, MeOD) 1.81-1.90 (m, 4 H) 2.61-2.69 (m, 4 H) 4.78 (s, 2 H) 7.76 (d, J = 8.08 Hz, 1 H) 8.06 (d, J = 6.06 Hz, 2 H) 8.20 (dd, J = 8.21, 1.89 Hz, 1 H) 8.30 (d, J = 1.77 Hz, 1 H) 6.61 (d, J = 6.32 Hz, 2 H) | | 4.48 | | 1000 |
| KK- 1l | 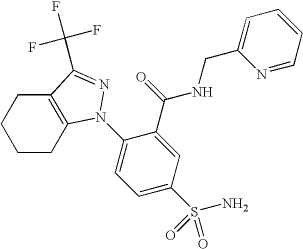 | 5-(aminosulfonyl)- N-(pyridin-2- ylmethyl)-2- ylmethyl)-2-[3- (trifluoromethyl)- 4,5,6,7-tetrahydro- 1H-indazol-1-yl] benzamide | 480 | ¹H NMR (400 MHz, MeOD) 1.94-2.00 (m, 4 H) 2.74-2.80 (m, 4 H) 4.93 (s, 2 H) 7.89 (d, J = 8.34 Hz, 1 H) 8.09-8.15 (m, 2 H) 8.34 (dd, J = 8.21, 2.15 Hz, 1 H) 8.43 (d, J = 2.02 Hz, 1 H) 8.69 (t, J = 8.59 Hz, 1H) 8.91 (dd, J = 6.19, 1.64 Hz, 1 H) | | 12.5 | | |
| KK- 1m | 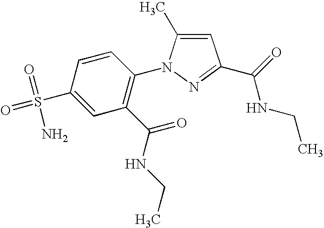 | 1-{4- (aminosulfonyl)-2- [(ethylamino) carbonyl]phenyl}- N-ethyl-5-methyl- 1H-pyrazole-3- carboxamide | 380 | ¹H NMR (400 MHz, DMSO-d6) 0.99 (t, J = 7.20 Hz, 3 H) 1.13 (t, J = 7.07 Hz, 3 H) 2.25 (s, 3 H) 3.10- 3.19 (m, 2 H) 3.26- 3.34 (m, 2 H) 6.65 (s, 1 H) 7.68 (s, 2 H) 7.81 (d, J = 8.84 Hz, 1 H) 7.98 (t, J = 5.68 Hz, 1 H) 8.06-8.10 (m, 2 H) 8.32 (t, J = 5.43 Hz, 1 H) | | 9.42 | | >5000 |
| KK- 1n | 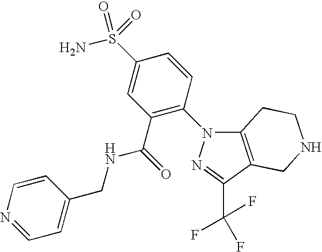 | 5-(aminosulfonyl)- N-(pyridin-4- ylmethyl)-2-[3- (trifluoromethyl)- 4,5,6,7-tetrahydro- 1H-pyrazolo[4,3- c]pyridin-1-yl] benzamide | 481 | ¹H NMR (400 MHz, DMSO-d6) 2.89- 2.98 (m, 2 H) 3.29- 3.37 (m, 2 H) 4.22- 4.29 (m, 2 H) 4.48 (d, J = 5.81 Hz, 2 H) 7.64 (d, J = 6.06 Hz, 2 H) 7.67 (s, 2 H) 7.75 (d, J = 8.08 Hz, 1 H) 8.06 (dd, J = 8.21, 2.15 Hz, 1 H) 8.15 (d, J = 2.02 Hz, 1 H) 8.69 (d, J= Hz, 2 H) 9.45 (t, J = 5.81 Hz, 1 H) 9.70 (br. s., 2 H) | | 14 | | >5000 |
| KK- 1o | 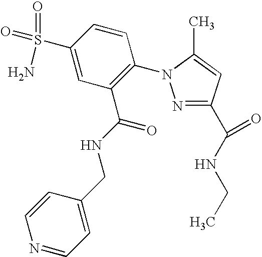 | 1-[4- (aminosulfonyl)-2- {[(pyridin-4- ylmethyl)amino] carbonyl}phenyl]- N-ethyl-5-methyl- 1H-pyrazole-3- carboxamide | 443 | ¹H NMR (400 MHz, DMSO-d6) 1.01 (t, J = 7.20 Hz, 3 H) 2.14 (s, 3 H) 3.14-3.26 (m, 2 H) 4.51 (d, J = 5.56 Hz, 2 H) 6.57 (s, 1 H) 7.62 (s, 2 H) 7.71 (br. s., 1 H) 7.75 (d, J = 8.34 Hz, 2 H) 8.02 (dd, J = 8.21, 2.15 Hz, 1 H) 8.07-8.13 (m, 2 H) 8.86 (br. s., 2 H) 9.31(t, J = 5.81 Hz, 1 H) | | 6.19 | | 4530 |
| KK- 1p | 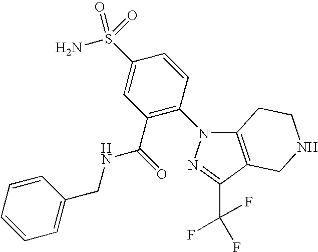 | 5-(aminosulfonyl)- N-benzyl-2-[3- (triflurormethyl)- 4,5,6,7-tetrahydro- 1H-pyrazolo[4,3- c]pyridin-1-yl] benzamide | 480 | ¹H NMR (400 MHz, DMSO-d6) anisotropism 2.76- 2.87 (m, 2 H) 3.39 (d, J = 11.12 Hz, 2 H) 4.15- 4.30 (m, 4 H) 7.03- 7.24 (m, 5 H) 7.58 (s, 2 H) 7.65-7.74 (m, 1 H) 7.93-8.02 (m, 2 H) 9.15 (s, 1 H) | | 26.9 | | >5000 |
| KK- 1r | 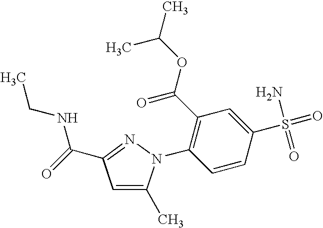 | isopropyl 5- (aminosulfonyl)-2- {3- [(ethylamino) carbonyl]- 5-methyl-1H- pyrazol-1- yl}benzamide | 395 | ¹H NMR (400 MHz, DMSO-d6) 0.93- 0.98 (m, 9 H) 2.11 (s, 3 H) 3.08-3.17 (m, 2 H) 4.78-4.87 (m, 1 H) 6.56 (s, 1 H) 7.60 (s, 2 H) 7.78 (d, J = 8.34 Hz, 1 H) 8.01 (t, J = 5.81 Hz, 1 H) 8.06 (dd, J = 8.34, 2.27 Hz, 1 H) 8.23 (d, J = 2.02 Hz, 1 H) | | 17.1 | | 1310 |
| KK- 1s | 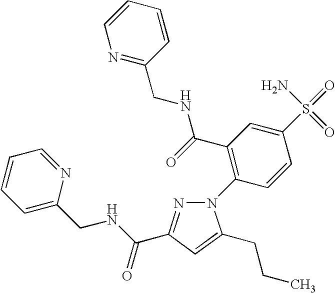 | 1-[4- (aminosulfonyl)-2- {[(pyridin-2- ylmethyl)amino] carbonyl}phenyl]-5- propyl-N-(pyridin- 2-ylmthyl)- 1H-pyrazole-3- carboxamide | 534 | ¹H NMR (400 MHz, DMSO-d6) 0.87 (t, J = 7.33 Hz, 3 H) 1.56 (q, J = 7.30 Hz, 2 H) 2.46-2.51 (m, 2 H) 4.72 (d, J = 5.61 Hz, 2 H) 4.83 (d, J = 5.81 Hz, 2 H) 6.74 (s, 1 H) 7.73 (br. s., 2 H) 7.79- 7.90 (m, 5 H) 8.12 (dd, J = 8.34, 2.02 Hz, 1 H) 8.29(d, J = 7.80 Hz, 1 H) 8.33 (d, J = 2.02 Hz, 1 H) 8.45 (t, J = 7.83 Hz, 1 H) 8.79 (d, J = 5.56 Hz, 1 H) 8.82 (d, J = 5.05 Hz, 1 H) 9.15 (t, J = 5.94 Hz, 1 H) 9.52 (t, J = 5.68 Hz, 1 H) | | 6.31 | | |
| KK- 1t | 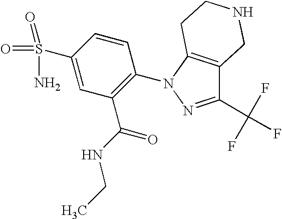 | 5-(aminosulfonyl)- N-ethyl-2-[3- (trifluoromethyl)- 4,5,6,7-tetrahydro- 1H-pyrazolo[4,3- c]pyridin-1-yl] benzamide | 418 | ¹H NMR (400 MHz, DMSO-d6) 1.22 (t, J = 7.20 Hz, 3 H) 3.19 (t, J = 5.68 Hz, 2 H) 3.30-3.40 (m, 2 H) 3.64 (t, J = 5.68 Hz, 2 H) 4.57 (s, 2 H) 7.92 (s, 2 H) 8.01 (d, J = 8.34 Hz, 1 H) 8.26 (d, J = 2.02 Hz, 1 H) 8.29 (dd, J = 8.30, 2.10 Hz, 1 H) 8.88 (t, J = 5.56Hz, 1 H) 9.83 (br. s., 2 H) | | 26 | | 1000 |
| KK- 1u | 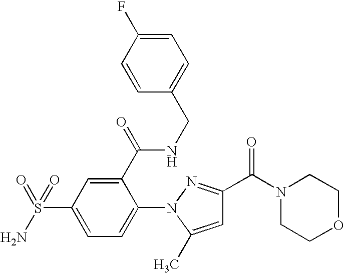 | 5-(aminosulfonyl)- N-(4- fluorobenzyl)-2-[5- methyl-3- (morpholin-4- ylcarbonyl)-1H- pyrazol-1-yl] benzamide | 502 | ¹H NMR (400 MHz, MeOD) 2.31 (s, 3 H) 3.52-3.60 (m, 2 H) 3.67-3.76 (m, 4 H) 3.87-3.95 (m, 2 H) 4.33-4.42 (m, 2 H) 6.62 (s, 1 H) 7.04 (t, J = 8.72 Hz, 2 H) 7.18- 7.24 (m, 2 H) 7.70 (d, J = 8.64 Hz, 1 H) 8.13- 8.18 (m, 2 H) 8.91 (t, J = 5.94 Hz, 1 H) | | 40.6 | | |
| LL-1 | 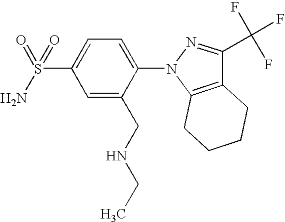 | 3- [(ethylamino) methyl]-4-[3- (trifluoromethyl)- 4,5,6,7-tetrahydro- 1H-indazol-1-yl] benzenesulfonamide | 403 | ¹H NMR (400 MHz, MeOD) 0.00 (t, J = 7.20 Hz, 3 H) 0.48- 0.57 (m, 4 H) 1.32- 1.39 (m, 4 H) 1.80 (q, J = 7.16 Hz, 2 H) 2.73 (s, 2 H) 6.43 (d, J = 8.34 Hz, 1 H) 6.79 (dd, J = 8.34, 2.02 Hz, 1 H) 8.92 (d, J = 1.77 Hz, 1 H) | | 25.9 | | 5000 |
| LL- 1a | 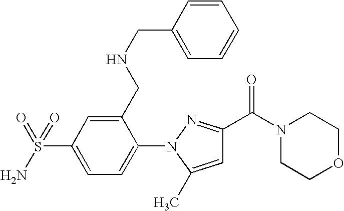 | 3- [(benzylamino) methyl]-4-(5-methyl-3- (morpholin-4- ylcarbonyl)- 1H-pyrazol-1- yl]benzenesulfonamide | 470 | ¹H NMR (400 MHz, MeOD) 2.27 (s, 3 H) 3.50-3.55 (m, 2 H) 3.60-3.68 (m, 6 H) 4.02 (s, 2 H) 4.22 (s, 2 H) 6.57 (s, 1 H) 7.32-7.37 (m, 3 H) 7.38-7.43 (m, 2 H) 7.66 (d, J = 8.34 Hz, 1 H) 8.05 (d, J = 8.34 Hz, 1 H) 8.20 (s, 1 H) | | 34.2 | | >5000 |
| ll-2 | 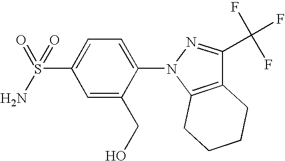 | 3- (hydroxymethyl)- 4-[3- (trifluoromethyl)- 4,5,6,7-tetrahydro- 1H-indazol-1-yl] benzenesulfonamide | 376 | ¹H NMR (400 MHz, DMSO-d6) 1.68- 1.78 (m, 4 H) 2.39- 2.46 (m, 2 H) 2.56- 2.61 (m, 2 H) 4.28 (s, 2 H) 5.48 (br. s., 1 H) 7.55 (s, 2 H) 7.61 (d, J = 8.06 Hz, 1 H) 7.83 (dd, J = 8.08, 2.27 Hz, 1 H) 8.16 (d, J = 2.02 Hz, 1 H) | | 7.53 | | 1000 |
| ll-2a | 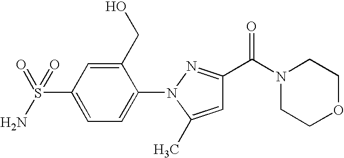 | 3- (hydroxymethyl)- 4-[5-methyl-3- (morpholin-4- ylcarbonyl)-1H- pyrazol-1-yl] benzenesulfonamide | 381 | ¹H NMR (400 MHz, DMSO-d6) 2.08 (s, 3 H) 2.98-3.05 (m, 1 H) 2.98-3.05 (m, 1 H) 3.48-3.60 (m, 4 H) 3.68-3.72 (m, 1 H) 3.81-3.86 (m, 2 H) 4.19 (s, 2 H) 6.54 (s, 1 H) 7.49 (s, 2 H) 7.54 (d, J = 8.34 Hz, 1 H) 7.79 (dd, J = 8.21, 2.15 Hz, 1 H) 8.11 (d, J = 1.77 Hz, 1 H) 8.97 (br. s., 1 H) | | 11.9 | | 1000 |
| mm- 1 | 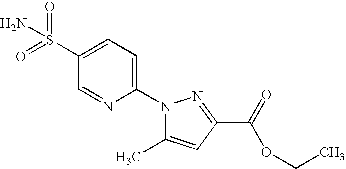 | ethyl 1-[5- (aminosulfonyl) pyridin-2-yl]-5-methyl- 1H-pyrazole-3- carboxylate | 311 | ¹H NMR (300 MHz, DMSO-d₆) 1.34 (t, J = 7.1 Hz, 3 H) 2.69 (s, 3 H) 4.35 (q, J = 7.1 hz, 2 H) 6.87 (s, 1 H) 7.74 (s, 2 H) 8.13 (d, J = 8.7 Hz, 1 H) 8.43 (dd, J = 8.7, 1.9 Hz, 1 H) 8.91 (d, J = 1.9 Hz, 1 H) | | 4.14 | | 1050 |
| MM- 1 | 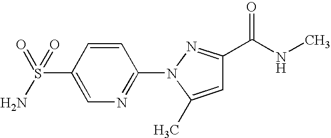 | 1-[5- (aminosulfonyl) pyridin-2-yl]-N-5- dimethyl-1H- pyrazole-3- carboxamide | 296 | ¹H NMR (300 MHz, DMSO-d₆) 2.69 (s, 3 H) 2.80 (d, J = 4.7 Hz, 3 H) 8.72 (d, J = 0.7 Hz, 1 H) 7.71 (s, 2 H) 8.20 (d, J = 8.7 Hz, 1 H) 8.38 (d, J = 4.7 Hz, 1 H) 8.42 (dd, J = 8.7, 2.1 Hz, 1 H) 8.69 (d, J = 2.1 Hz, 1 H) | | 9.54 | | |
| MM- 1a | 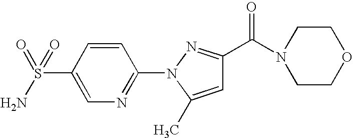 | 6-[5-methyl-3- (morpholin-4- ylcarbonyl)-1H- pyrazol-1- yl]pyridine-3- sulfonamide | 352 | ¹H NMR (300 MHz, DMSO-d₆) 2.69 (s, 3 H) 3.59-3.73 (m, 6 H) 3.92 (t, J = 4.3 Hz, 2 H) 6.68 (d, J = 0.7 Hz, 1 H) 7.71 (s, 2 H) 8.08 (d, J = 8.7 Hz, 1 H) 8.38 (dd, J = 8.67, 2.2 Hz, 1 H) 8.89 (d, J = 2.2 Hz, 1 H) | | 7.06 | | |
| MM- 1b | 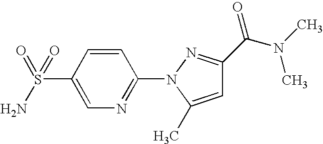 | 1-[5- (aminosulfonyl) pyridin-2-yl]-N,N,5- trimethyl-1H- pyrazole-3- carboxamide | 310 | ¹H NMR (300 MHz, DMSO-d₆) 2.69 (d, J = 0.8 Hz, 3 H) 3.03 (s, 3 H) 3.29 (s, 3 H) 6.65 (d, J = 0.8 Hz, 1 H) 7.70 (s, 2 H) 8.08 (d, J = 8.7 Hz, 1 H) 8.38 (dd, J = 8.7, 2.2 Hz, 1 H) 8.88 (d, J = 2.2 Hz, 1 H) | | 5.02 | 5.9 | 2390 |
| MM- 1c | 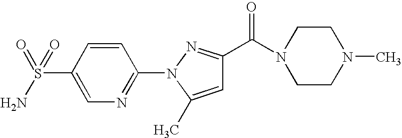 | 6-{5-methyl-3-[(4- methylpiperazin-1- yl)carbonyl]-1H- pyrazol-1- yl}pyridine-3- sulfonamide | 365 | ¹H NMR (300 MHz, DMSO-d₆) 2.70 (s, 3 H) 2.83 (s, 3 H) 6.74 (d, J = 0.7 Hz, 1 H) 7.75 (s, 2 H) 8.11 (d, J = 8.7 Hz, 1 H) 8.40 (dd, J = 8.67, 2.1 Hz, 1 H) 8.90 (d, J = 2.1 Hz, 1 H) | | 6.77 | | 2950 |
| MM- 1d | 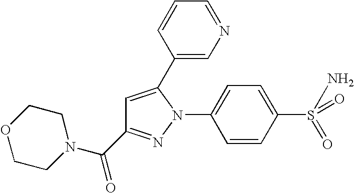 | 4-[3-(morpholin-4- ylcarbonyl)-5- pyridin-3-yl-1H- pyrazol-1- yl]benzenesulfonamide | 414 | ¹H NMR (300 MHz, DMSO-d₆) 3.60-3.68 (m, 2 H) 3.95-4.04 (m, 2 H) 4.50-4.70 (m, 4 H) 7.21 (s, 1 H) 7.50-7.67 (m, 5 H) 7.89 (d,J = 8.67 Hz, 2 H) 8.67-8.74 (m, 2 H) | | 267 | | |
| mm- 2 | 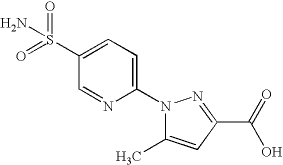 | 1-[5- (aminosulfonyl) pyridin-2-yl]-5-methyl- 1H-pyrazole-3- carboxylic acid | 283 | ¹H NMR (400 MHz, DMSO-d₆) 2.46 (s, 3 H) 6.58 (s, 1 H) 7.51 (s, 2 H) 7.90 (d, J = 8.7 Hz, 1 H) 8.20 (dd, J = 8.7, 2.3 Hz, 1 H) 8.68 (d, J = 2.3 Hz, 1 H) 12.91 (s, 1 H) | | 13.9 | | |
| mm- 2a | 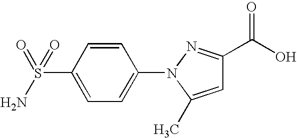 | 1-[4- (aminosulfonyl) phenyl]- 5-methyl-1H- pyrazole-3- carboxylic acid | 282 | ¹H NMR (400 MHz, DMSO-d6) 2.40 (s, 3 H) 6.76 (s, 1 H) 7.53 (s, 2 H) 7.82 (d, J = 8.59 Hz, 2 H) 7.99 (d, J = 8.59 Hz, 2 H) 12.89 (s, 1 H) | | 29.4 | | 5000 |
| mm- 2b | 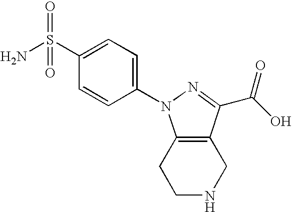 | 1-[4- (aminosulfonyl) phenyl]-4,5,6,7- tetrahydro-1H- pyrazolo[4,3- c]pyridine-3- carboxylic acid | 323 | ¹H NMR (300 MHz, DMSO-d₆) 3.20 (t, J = 5.6 Hz, 2 H) 4.35 (s, 2 H) 7.58 (s, 2 H) 7.86 (d, J = 8.7 Hz, 2 H) 8.03 (d, J = 8.7 Hz, 2 H | | | | |
| NN- 1 | 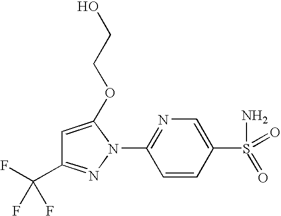 | 6-[5-(2- hydroxyethoxy)-3- (trifluoromethyl)- 1H-pyrazol-1- yl]pyridine-3- sulfonamide | 363 | ¹H NMR (300 MHz, DMSO-d₆) 3.75 (q, J = 4.8 Hz, 2 H) 4.33 (t, J = 4.8 Hz, 2 H) 5.01 (t, J = 5.5 Hz, 1 H) 6.57 (s, 1 H) 7.75 (s, 2 H) 8.00 (d, J = 8.5 Hz, 1 H) 8.40 (dd, J = 8.5, 2.0 Hz, 1 H) 8.96 (d, J = 2.0 Hz, 1 H) | | 19.8 | | |
| NN- 1a | 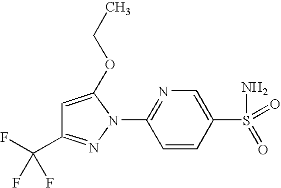 | 5-[5-ethoxy-3- (trifluoromethyl)- 1H-pyrazol-1- yl]pyridine-3- sulfonamide | 337 | ¹H NMR (300 MHz, DMSO-d₆) 1.39 (t, J = 7.0 Hz, 3 H) 4.35 (q, J = 7.0 Hz, 2 H) 8.56 (s, 1 H) 7.74 (s, 2 H) 7.95 (d, J = 8.5 Hz, 1 H) 8.41 (dd, J = 8.5, 2.1 Hz, 1 H) 8.96 (d, J = 2.1 Hz, 1 H) | | 5.67 | | |
| NN- 1b | 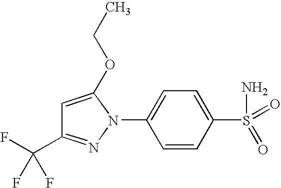 | 4-[5-ethoxy-3- (trifluoromethyl)- 1H-pyrazol-1- yl]benzenesulfonamide | 336 | ¹H NMR (300 MHz, DMSO-d₆) 1.41 (t, J = 7.0 Hz, 3 H) 4.35 (q, J = 7.0 Hz, 2 H) 6.55 (s, 1 H) 7.50 (s, 2 H) 7.93 (d, J = 9.0 Hz, 2 H) 7.99 (d, J = 9.0 Hz, 2 H) | | 6.05 | | |
| NN- 1c | 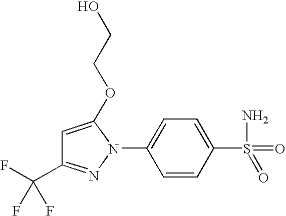 | 4-[5-(2- hydroxyethoxy)-3- (trifluoromethyl)- 1H-pyrazol-1- yl]benzenesulfonamide | 352 | ¹H NMR (300 MHz, DMSO-d₆) 3.78 (q, J = 5.3 Hz, 2 H) 4.33 (t, J = 4.5 Hz, 2 H) 5.08 (t, J = 5.5 Hz, 1 H) 6.57 (s, 1 H) 7.51 (s, 2 H) 7.98 (s, 4 H) | | 14 | | |
| OO- 1 | 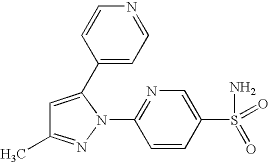 | 6-(3-methyl-5- pyridin-4-yl-1H- pyrazol-1- yl)pyridine-3- sulfonamide | 316 | ¹H NMR (300 MHz, DMSO-d₆) 2.35 (s, 3 H) 6.71 (s, 1 H) 7.32 (dd, J = 6.9, 1.7 Hz, Hz, 2 H) 7.66 (s, 2 H) 8.04 (d, J = 8.7 Hz, 1 H) 8.37 (dd, J = 8.7, 2.1 Hz, 1 H) 8.52 (d, J = 2.1 Hz, 1 H) 8.58 (dd, J = 6.9, 1.7 Hz, 2 H) | | 446 | | |
| OO- 1a | 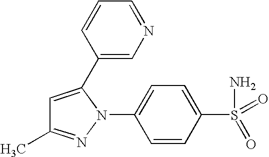 | 4-(3-methyl-5- pyridin-3-yl-1H- pyrazol-1- yl)benzenesulfonamide | 315 | ¹H NMR (300 MHz, DMSO-d₆) 2.33 (s, 3 H) 6.66 (s, 1 H) 7.41- 7.52 (m, 5 H) 7.63- 7.69 (m, 1 H) 7.84 (d, J = 8.7 Hz, 2 H) 8.50 (d, J = 1.7 Hz, 1H) 8.59 (dd, J = 4.8, 1.7 Hz, 1 H) | | 89.8 | | |
| OO- 1b | 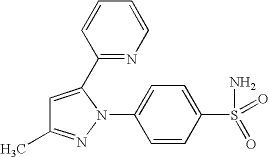 | 4-(3-methyl-5- pyridin-2-yl-1H- pyrazol-1- yl)benzenesulfonamide | 315 | ¹H NMR (300 MHz, DMSO-d₆) 2.33 (s, 3 H) 6.73 (s, 1 H) 7.35- 7.42 (m, 3 H) 7.45 (s, 2 H) 7.60 (d, J = 7.9 Hz, 1 H) 7.80 (d, J = 8.7 Hz, 2 H) 7.90 (td, J = 7.7, 1.7 Hz, 1 H) 8.49 (dd, J = 4.0, 1.7 Hz, 1 H) | | 245 | | |
| OO- 1c | 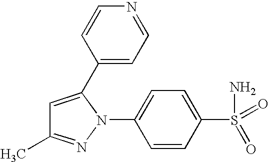 | 4-(3-methyl-5- pyridin-4-yl-1H- pyrazol-1- yl)benzenesulfonamide | 315 | ¹H NMR (300 MHz, DMSO-d₆) 2.33 (s, 3 H) 6.73 (s, 1 H) 7.25 (dd, J = 4.5, 1.5 Hz, 2 H) 7.44-7.53 (m, 4 H) 7.88 (d, J = 8.67 Hz, 2 H) 8.60 (dd, J = 4.5, 1.5 Hz, 2 H) | | 145 | | |
| OO- 1d | 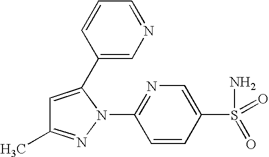 | 6-(3-methyl-5- pyridin-3-yl-1H- pyrazol-1- yl)pyridine-3- sulfonamide | 316 | ¹H NMR (300 MHz, DMSO-d₆) 2.56 (s, 3 H) 7.79 (dd, J = 8.0, 5.0 hz, 1 H) 7.84 (s, 2 H) 8.16 (dt, J = 8.0, 1.9 Hz, 1 H) 8.26 (d, J = 8.7 Hz, 1 H) 8.55 (dd, J = 8.7, 2.4 Hz, 1 H) 8.67 (d, J = 2.4 Hz, 1 H) 8.84-8.91 (m, 2 H) | | 576 | | |
| OO- 1e | 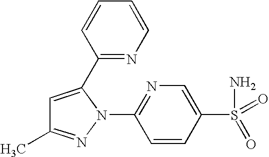 | 6-(3-methyl-5- pyridin-2-yl-1H- pyrazol-1- yl)pyridine-3- sulfonamide | 316 | ¹H NMR (300 MHz, DMSO-d₆) 2.35 (s, 3 H) 6.73 (s, 1 H) 7.33- 7.40 (m, 1 H) 7.60 (d, J = 7.7 Hz, 1 H) 7.66 (s, 2 H) 7.87 (td, J = 7.7, 1.7 Hz 1 H) 7.93 (d, J = 8.7 Hz, 1 H) 8.33 (dd, J = 8.7, 1.7 Hz, 1 H) 8.45 (dd, J = 4.9, 1.7 Hz, 1 H) 8.50 (d, J = 1.7 Hz, 1 H) | | 410 | | |
| PP-1 | 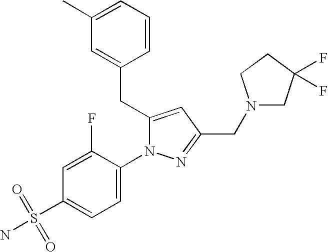 | 4-{2-[(3,3- difluoropyrrolidin- 1-yl)methyl]-5-{3- methylbenzyl)-1H- pyrazol-1-yl]-3- fluorobenzenesulfonamide | 465 | ¹H NMR (400 MHz, MeOD-D4) 2.20 (s, 3H) 2.67 (bs, 2H), 3.80 (bs, 2H), 3.94 (s, 2H), 3.97 (bs, 2H), 4.57 (s, 2H), 6.51 (s, 1H), 6.76-6.77 (m, 2H), 6.95-6.97 (m, 1H), 7.04-7.06 (m, 1H), 7.55-7.59 (m, 1H), 7.78-7.81 (m, 2H) | | | 0.1 | 114 |
| PP- 1a | 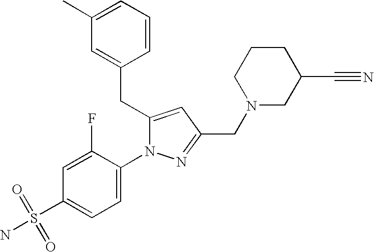 | 4-[3-[(3- cyanopiperidin-1- yl)methyl]-5-(3- methylbenzyl)-1H- pyrazol-1-yl]-3- fluorobenzene- sulfonamide | 468 | ¹H NMR (400 MHz, MeOD-D4) 1.62-1.70 (m, 2H), 1.76-1.83 (m, 2H), 2.20 (s, 3H), 2.47-2.61 (m, 3H), 2.70-2.73 (m, 1H), 2.88-2.93 (m, 2H), 3.55-3.64 (dd, J1 = 12 Hz, J2 = 8 Hz, 2H), 3.90 (s, 2H), 8.32 (s, 1H), 6.74-6.76 (m, 2H), 6.94-6.96 (m, 1H), 7.03-7.07 (m, 1H), 7.48-7.50 (t, J = 8 HZ, 1H), 7.75-7.77 (m, 2H) | | | 0.1 | 168 |
| PP- 1b | 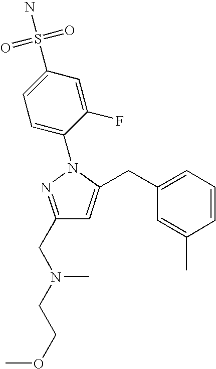 | 3-fluoro-4-[3-{[(2- methoxyethyl) (methyl)amino]methyl}- 5-(3- methylbenzyl)-1H- pyrazol-1- yl]benzenesulfonamide | 447 | ¹H NMR (400 MHz, MeOH-D4) 2.20 (s, 3H), 2.92 (s, 3H), 3.39 (s, 3H), 3.47- 3.53 (m, 1H), 3.72- 3.74 (t, J = 4 Hz, 2H), 3.94 (s, 2H), 4.35- 4.45 (dd, J1 = 12, J2 = 8 Hz, 2 H), 6.50 (s, 1H), 6.76-6.78 (m, 2H), 6.96-6.98 (m, 1H), 7.04-7.08 (t, J = 8 Hz, 1H) 7.54-7.58 (t, J= 8 Hz, 1H) 7.78- 7.80 (m, 2H) | | | 0.1 | 145 |
| pp- 2a | 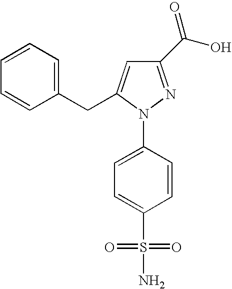 | 1-[4- (aminosulfonyl) phenyl]-5-benzyl-1H- pyrazole-3- carboxylic acid | 358 | ¹H NMR (300 MHz, DMSO-d₆) 3.33 (s, 2 H) 4.16 (s, 2 H) 6.53 (s, 1 H) 7.10-7.18 (m, 2 H) 7.20-7.35 (m, 3 H) 7.54 (s, 2 H) 7.78 (d, J = 8.7 Hz, 2 H) 7.98 (d, J = 8.7 Hz, 2 H) | | 2.42 | 1.6 | 492 |
| pp- 2b | 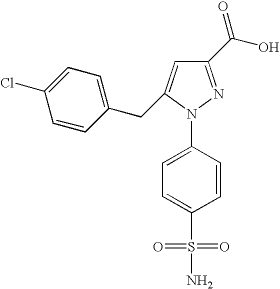 | 1-[4- (aminosulfonyl) phenyl]-5-(4- chlorobenzyl]-1H- pyrazole-3- carboxylic acid | 392 | ¹H NMR (300 MHz, DMSO-d₆) 4.17 (s, 2 H) 6.54 (s, 1 H) 7.17 (d, J = 8.5 Hz, 2 H) 7.36 (d, J = 8.5 Hz, 2 H) 7.54 (s, 2 H) 7.77 (d, J = 8.7 Hz, 2 H) 7.98 (d,J = 8.7 Hz, 2 H) | | 3.15 | | 459 |
| QQ- 1 | 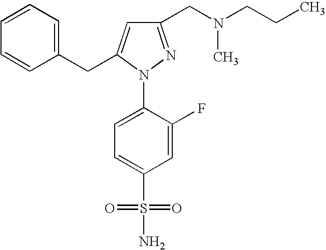 | 4-(5-benzyl-3- {[methyl(propyl) amino]methyl}-1H- pyrazol-1-yl)-S- fluorobenzene- sulfonamide | 417 | ¹H NMR (300 MHz, DMSO-d₆) 0.90 (t, J = 7.3 Hz, 3 H) 1.64- 1.79 (m, J = 6.1 Hz, 2 H) 2.74 (s, 3 H) 2.91- 3.12 (m, 2 H) 3.99 (s, 2 H) 4.23-4.41 (m, 2 H) 6.50 (s, 1 H) 7.03- 7.09 (m, 2 H) 7.20- 7.30 (m, 3 H) 7.71 (s, 2 H) 7.77-7.82 (m, 2 H) 7.85 (d, J = 10.4 Hz, 1 H) | | 2.4 | 0.3 | 301 |
| QQ- 1a | 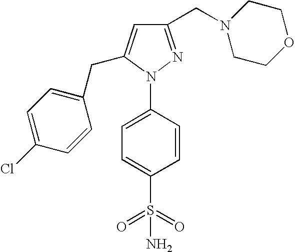 | 4-[5-(4- chlorobenzyl)-3- (morpholin-4- ylmethyl)-1H- pyrazol-1- yl]benzenesulfon- amide | 447 | ¹H NMR (300 MHz, DMSO-d₆) 2.41 (t, J = 4.5 Hz, 4 H) 3.46 (s, 2 H) 3.57 (t, J = 4.5 Hz, 4 H) 4.17 (s, 2 H) 6.05 (s, 1 H) 7.18 (d, J = 8.4 Hz, 2 H) 7.36 (d, J = 8.4 Hz, 2 H) 7.48 (s, 2 H) 7.72 (d, J = 8.5 Hz, 2 H) 7.93 (d, J = 8.5 Hz, 2 H) | | 2.03 | 0.8 | 563 |
| QQ- 1b | 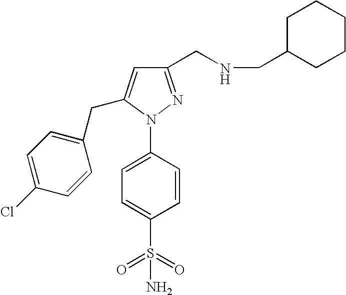 | 4-[5-(4- chlorobenzyl)-3- {[(cyclohexylmethyl) amino]methyl}- 1H-pyrazol-1- yl]benzenesulfon- amide | 473 | ¹H NMR (300 MHz, DMSO-d₆) 0.77- 0.95 (m, 2 H) 1.09- 1.28 (m, 3 H) 1.31- 1.46 (m, 1 H) 1.58- 1.79 (m, 5 H) 2.38 (d, J = 6.6 Hz, 2 H) 3.83 (s, 2 H) 4.16 (s, 2 H) 6.07 (s, 1 H) 7.19 (d, J = 8.5 Hz, 2 H) 7.36 (d, J = 8.5 Hz, 2 H) 7.47 (s, 1 H) 7.71 (d, J = 8.7 Hz, 2 H) 7.92 (d, J= 8.7 Hz, 2 H) | | 4.03 | | 281 |
| QQ- 1c | 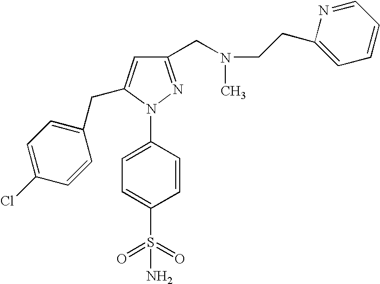 | 4-[5-(4- chlorobenzyl)-3- {[methyl(2-pyridin- 2- ylethyl)amino] methyl}-1H-pyrazol-1- yl]benzenesulfon- amide | 496 | ¹H NMR (300 MHz, DMSO-d₆) 2.86 (s, 3 H) 4.19 (s, 2 H) 4.42 (s, 2 H) 6.41 (s, 1 H) 7.20 (d, J = 8.4 Hz, 2 H) 7.37 (d, J = 8.4 Hz, 2 H) 7.50-7.63 (m, 4 H) 7.77 (d, J = 8.5 Hz, 2 H) 7.98 (d, J = 8.5 Hz, 2 H) 8.06 (t, J = 7.9 Hz, 1 H) 8.63 (d, J = 4.7 Hz, 1 H) | | 2.07 | 0.7 | 380 |
| QQ- 1d | 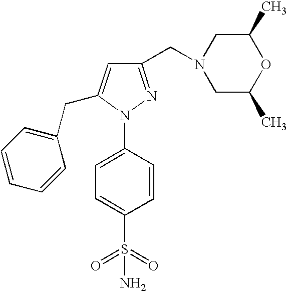 | 4-(5-benzyl-3- {[(2R,6S)-2,6- dimethylmorpholin- 4-yl]methyl}-1H- pyrazol-1- yl)benzenesulfon- amide | 441 | ¹H NMR (300 MHz, DMSO-d₆) 1.14 (d, J = 6.2 Hz, 6 H) 2.64- 2.82 (m, 2 H) 3.89 (bs, 2 H) 4.18 (s, 2 H) 4.34 (bs, 2 H) 6.37 (s, 1 H) 7.23-7.29 (m, 1 H) 7.29-7.37 (m, J = 7.3 Hz, 2 H) 7.54 (s, 2 H) 7.78 (d, J = 8.7 Hz, 2 H) 7.98 (d, J = 8.7 Hz, 2 H) | | 2.39 | 0.8 | 400 |
| QQ- 1e | 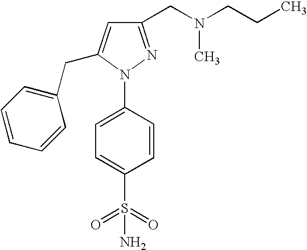 | 4-(5-benzyl-3- {[methyl(propyl) amino]methyl}-1H- pyrazol-1- yl)benzenesulfon- amide | 399 | ¹H NMR (300 MHz, DMSO-d₆) 0.90 (t, J = 7.3 Hz, 3 H) 1.64- 1.82 (m, 2 H) 2.75 (s, 3 H) 3.03 (bs, 2 H) 4.18 (s, 2 H) 4.22- 4.45 (m, 2 H) 6.37 (s, 1 H) 7.17 (d, J = 7.2 Hz, 2 H) 7.22-7.36 (m, 3 H) 7.54 (s, 2 H) 7.77 (d, J = 8.5 Hz, 2 H) 7.98 (d, J = 8.5 Hz, 2 H) | | 2.02 | 1 | 379 |
| QQ- 1f | 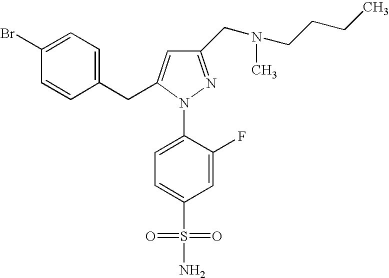 | 4-[5-(4- brromobenzyl)-3- {[butyl(methyl) amino]methyl}-1H- pyrazol-3-yl]-3- fluorobenzenesul- fonamide | 511 | ¹H NMR (300 MHz, DMSO-d₆) 0.89 (t, J = 7.3 Hz, 3 H) 1.22- 1.38 (m, 2 H) 1.61- 1.78 (m, 2 H) 2.74 (d, J = 4.7 Hz, 3 H) 2.92- 3.17 (m, 2 H) 3.97 (s, 2 H) 4.22-4.41 (m, 2 H) 6.50 (s, 1 H) 7.04 (d, J = 8.5 Hz, 2 H) 7.47 (d, J = 8.5 Hz, 2 H) 7.72 (s, 2 H) 7.78- 7.90 (m, 3 H) | | 2.05 | 0.3 | 171 |
| QQ- 1g | 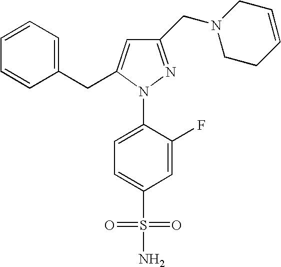 | 4-[5-benzyl-3-(3,6- dihydropyridin- 1(2H)-ylmethyl)- 1H-pyrazol-1-yl]-3- fluorobenzenesul- fonamide | 427 | ¹H NMR (300 MHz, DMSO-d₆) 2.25-2.40 (m, 2 H) 3.07-3.18 (m, 1 H) 3.47-3.58 (m, 1 H) 3.69 (bs, 2 H) 3.99 (s, 2 H) 4.37 (s, 2 H) 5.72 (d, J = 10.3 Hz, 1 H) 5.92 (d, J = 10.3 Hz, 1 H) 6.53 (s, 1 H) 7.03- 7.10 (m, 2 H) 7.19- 7.32 (m, 3 H) 7.72 (s, 2 H) 7.78-7.83 (m, 2 H) 7.85 (d, J = 10.0 Hz, 1H) | | 2.4 | 0.2 | 354 |
| - 1h | 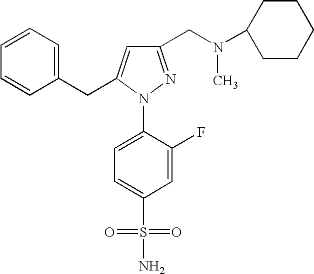 | 4-(5-benzyl-3- {[cyclohexyl(methyl) amino]methyl}- 1H-pyrazol-1-yl)- 3- fluorobenzenesul- fonamide | 457 | ¹H NMR (300 MHz, DMSO-d₆) 1.33-1.58 (m, 3 H) 1.57-1.68 (m, 1 H) 1.77-1.90 (m, 2 H) 1.99-2.15 (m, 2 H) 2.71 (s, 3 H) 3.08-3.24 (m, 1 H) 3.99 (s, 2 H) 4.21- 4.47 (m, 2 H) 6.51 (s, 1 H) 7.03-7.09 (m, 2 H) 7.19-7.32 (m, 3 H) 7.71 (s, 2 H) 7.75- 7.82 (m, 2 H) 7.85 (d, J = 9.8 Hz, 1 H) | | 1.78 | 0.2 | 186 |
| QQ- 1i | 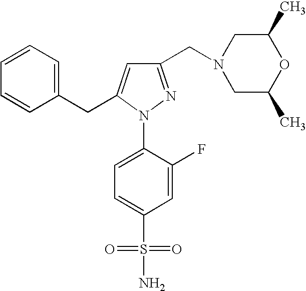 | 4-(5-benzyl-3- {[(2R,6S)-2,6- dimethylmorpholin- 4-yl]methyl}-1H- pyrazol-1-yl)-3- fluorobenzenesul- fonamide | 459 | ¹H NMR (300 MHz, DMSO-d₆) 1.14 (d, J = 6.4 Hz, 6 H) 2.60- 2.81 (m, 2 H) 3.89- 4.02 (m, 2 H) 3.99 (s, 2 H) 4.33 (s, 2 H) 6.55 (s, 1 H) 7.03- 7.10 (m, 2 H) 7.20- 7.31 (m, 3 H) 7.72 (s, 2 H) 7.78-7.89 (m, 3 H) | | 2.25 | 0.2 | 480 |
| QQ- 1j | 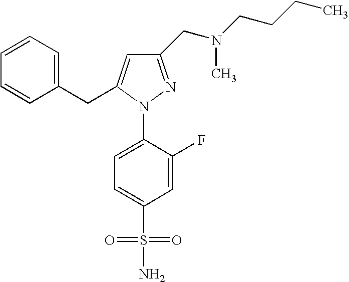 | 4-(5-benzyl-3- {[butyl(methyl) amino]methyl}-1H- pyrazol-1-yl)-3- fluorobenzenesul- fonamide | 431 | ¹H NMR (300 MHz, DMSO-d₆) 0.89 (t, J = 7.3 Hz, 3 H) 1.25- 1.37 (m, 2 H) 1.61- 1.75 (m, 2 H) 2.74 (s, 3 H) 2.94-3.17 (m, 2 H) 3.99 (s, 2 H) 4.25- 4.41 (m, 2 H) 6.51 (s, 1 H) 7.02-7.10 (m, 2- H) 7.20-7.31 (m, 3 H) 7.71 (s, 2 H) 7.77- 7.88 (m, 3 H) | | 2.44 | 0.3 | 278 |
| QQ- 1k | 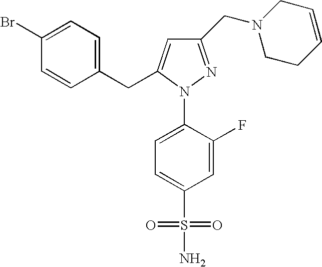 | 4-[5-(4- bromobenzyl)-3- (3,6- dihydropyridin- 1(2H)-ylmethyl)- 1H-pyrazol-1-yl]-3- fluorobenzenesul- fonamide | 507 | ¹H NMR (300 MHz, DMSO-d₆) 2.32 (d, J = 18.1 Hz, 1 H) 3.03- 3.19 (m, 1 H) 3.45- 3.56 (m, 1 H) 3.68 (s, 2 H) 3.97 (s, 2 H) 4.37 (d, J = 4.0 Hz, 2 H) 5.72 (d, J = 10.4 Hz, 1 H) 5.92 (d, J = 10.4 Hz, 1 H) 6.53 (s, 1 H) 7.05 (d, J = 8.5 Hz, 2 H) 7.48 (d, J = 8.5 Hz, 2H) 7.73 (s, 2 H) 7.80-7.91 (m, 3 H) | | 2.32 | 0.2 | 253 |
| QQ- 1l | 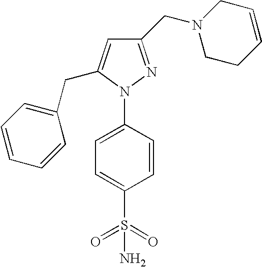 | 4-[5-benzyl-3-(3,6- dihydropyridin- 1(2H)-ylmethyl)- 1H-pyrazol-1- yl]benzenesulfon- amine | 409 | ¹H NMR (300 MHz, DMSO-d₆) 2.27-2.39 (m, 1 H) 2.40-2.49 (m, 1 H) 3.04-3.22 (m, 1 H) 3.46-3.58 (m, 1 H) 3.70 (br. s., 2 H) 4.18 (s, 2 H) 4.38 (s, 2 H) 5.72 (d, J = 10.0 Hz, 1 H) 5.92 (d, J = 10.0 Hz, 1 H) 6.39 (s, 1 H) 7.15- 7.21 (m, 2 H) 7.22- 7.37 (m, 3 H) 7.54 (s, 2 H) 7.78 (d, J = 8.7 Hz, 2 H)7.98 (d, J = 8.7 Hz, 2 H) | | 1.59 | 1.4 | 552 |
| QQ- 1m | 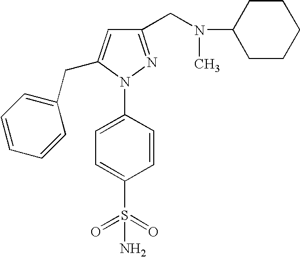 | 4-(5-benzyl-3- {[cyclohexyl(methyl) amino]methyl}- 1H-pyrazol-1- yl)benzenesulfon- amide | 439 | ¹H NMR (300 MHz, DMSO-d₆) 1.06-1.35 (m, 3 H) 1.37-1.55 (m, 2 H) 1.57-1.68 (m, 1 H) 1.83 (d, J = 12.1 Hz, 2 H) 2.02- 2.20 (m, 2 H) 2.70 (s, 3 H) 3.10-3.26 (m, 1 H) 4.15-4.30 (m, 3 H) 4.33-4.45 (m, 3 H) 6.43 (s, 1 H) 7.13- 7.19 (m, 2 H) 7.23- 7.36 (m, 3 H) 7.54 (s, 2 H) 7.76 (d, J = 8.7 Hz, 2 H) 7.98(d, J = 8.7 Hz, 2 H) | | 2.31 | 0.7 | 314 |
| QQ- 1n | 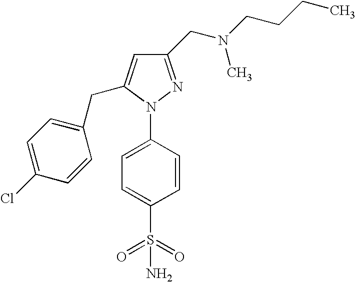 | 4-[3- {[butyl(methyl) amino]methyl}-5-(4- chlorobenzyl)-1H- pyrazol-1- yl]benzenesulfon- amide | 447 | ¹H NMR (300 MHz, DMSO-d₆) 0.86 (t, J = 7.3 Hz, 3 H) 1.21- 1.34 (m, 2 H) 1.37- 1.52 (m, 2 H) 2.19 (s, 3 H) 2.30-2.45 (m, 2 H) 3.50 (s, 2 H) 4.17 (s, 2 H) 6.03 (s, 1 H) 7.18 (d, J = 8.3 Hz, 2 H) 7.36 (d, J = 8.3 Hz, 2 H) 7.48 (s, 2 H) 7.71 (d, J = 8.5 Hz, 2 H) 7.93 (d, J = 8.5Hz, 2 H) | | 4.23 | | 455 |
| QQ- 1o | 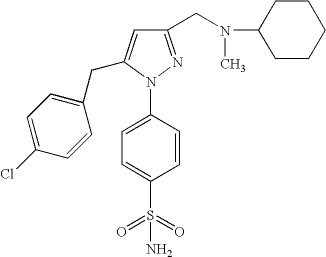 | 4-[5-(4- chlorobenzyl)-3- {[cyclohexyl(methyl) amino]methyl}- 1H-pyrazol-1- yl]benzenesulfon- amide | 473 | ¹H NMR (300 MHz, DMSO-d₆) 1.15-1.35 (m, 4 H) 1.52-1.65 (m, 1 H) 1.68-1.84 (m, 4 H) 2.17 (s, 3 H) 2.32-2.48 (m, 1 H) 3.56 (s, 2 H) 4.17 (s, 2 H) 6.02 (s, 1 H) 7.18 (d, J = 8.5 Hz, 2 H) 7.36 (d, J = 8.6 Hz, 2 H) 7.47 (s, 2 H) 7.71 (d, J = 8.6 Hz, 2 H) 7.93 (d, J = 8.8 Hz, 2H) | | 2.87 | 0.6 | 308 |
| qq-3 | 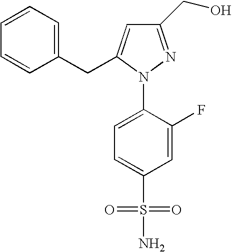 | 4-[5-benzyl-3- (hydroxymethyl)- 1H-pyrazol-1-yl]-3- fluorobenzenesulfon- amide | 362 | ¹H NMR (400 MHz, DMSO-d6) 3.88 (s, 2 H) 4.40 (s, 2 H) 6.13 (s, 1 H) 7.02 (d, J = 7.1 Hz, 2 H) 7.18 (d, J = 7.1 Hz, 1 H) 7.23 (t, J = 7.1 Hz, 2 H) 7.58 (t, J = 8.1 Hz, 1 H) 7.69 (d, J = 8.1 Hz, 1 H) 7.73 (dd, J = 9.7, 1.4 Hz, 1 H) | | 2.84 | 0.1 | 372 |
| qq- 3a | 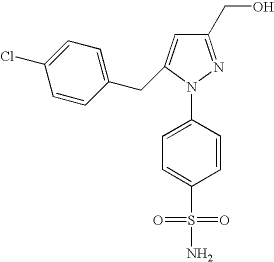 | 4-[5-(4- chlorobenzyl)-3- (hydroxymethyl)- 1H-pyrazol-1- yl]benzenesulfon- amide | 378 | ¹H NMR (300 MHz, DMSO-d₆) 4.16 (s, 2 H) 4.44 (d, J = 5.6 Hz, 2 H) 6.12 (t, J = 5.8 Hz, 1 H) 6.08 (s, 1 H) 7.19 (d, J = 8.5 Hz, 2 H) 7.37 (d, J = 8.5 Hz, 2 H) 7.48 (s, 2 H) 7.71 (d, J = 8.7 Hz, 2 H) 7.93 (d, J = 8.7 Hz, 2 H) | | 2.12 | 0.2 | 284 |
| qq- 3b | 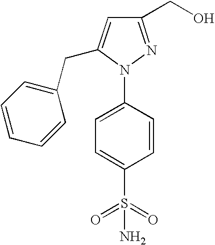 | 4-[5-benzyl-3- (hydroxymethyl)- 1H-pyrazol-1- yl]benzenesulfon- amide | 344 | ¹H NMR (300 MHz, DMSO-d₆) 4.15 (s, 2 H) 4.44 (s, 2 H) 6.08 (s, 1 H) 7.14-7.20 (m, 2 H) 7.20-7.27 (m, 1 H) 7.28-7.35 (m, 2 H) 7.48 (s, 2 H) 7.72 (d, J = 8.9 Hz, 2 H) 7.93 (d, J = 8.9 Hz, 2 H) | | 1.91 | 0.2 | 436 |
| qq- 3c | 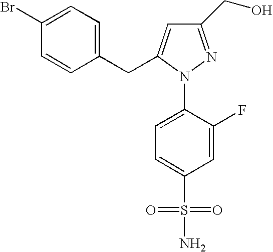 | 4-[5-(4- bromobenzyl)-3- (hydroxymethyl)- 1H-pyrazol-1-yl]-3- fluorobenzenesulfon- amide | 442 | ¹H NMR (300 MHz, DMSO-d₆) 2.60 (s, 1 H) 3.95 (s, 2 H) 4.48 (s, 2 H) 6.23 (s, 1 H) 7.06 (d, J = 8.3 Hz, 2 H) 7.49 (d, J = 8.3 Hz, 2 H) 7.69-7.91 (m, 5 H) | | 3.06 | 0.1 | 468 |
| qq- 3d | 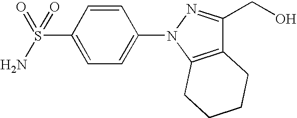 | 4-[3- (hydroxymethyl)- 4,5,6,7-tetrahydro- 1H-indazol-1-yl] benzenesulfon- amide | 308 | ¹H NMR (400 MHz, DMSO-d6) 1.69- 1.80 (m, 2 H) 2.54 (t, J = 5.56 Hz, 2 H) 2.81 (t, J = 4.80 Hz, 2 H) 4.45 (d, J = 5.56 Hz, 2 H) 4.99 (t, J = 5.58 Hz, 1 H) 7.42 (s, 2 H) 7.75 (d, J = 8.59 Hz, 2 H) 7.91 (d, J = 8.59 Hz, 2 H) | | 5.51 | | 5000 |

| | | | LRMS |
|---|---|---|---|
| Example | | | m/z |
| number | STRUCTURE | IUPAC NAME | (M + H)⁺ |
| bbb-5 | 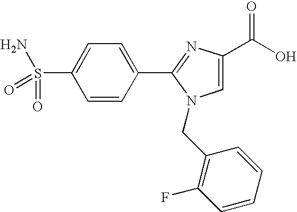 | 2-[4- (aminosulfonyl) phenyl]-1-(2- fluorobenzyl)- 1H-imidazole-4- carboxylic acid | 376 |
| BBB-1 | 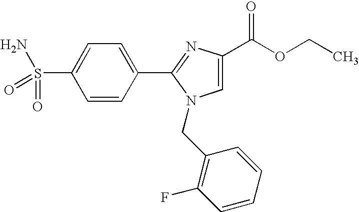 | ethyl 2-[4- (aminosulfonyl) phenyl]-1-(2- fluorobenzyl)- 1H-imidazole-4- carboxylate | 404 |
| AAA-1a | 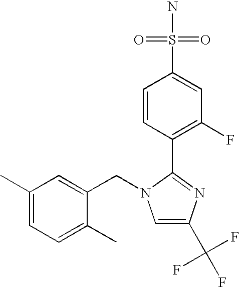 | 4-[1-(2,5- dimethylbenzyl)- 4- (trifluoromethyl)- 1H-imidazol-2- yl]-3- fluorobenzene- sulfonamide | 428 |
| AAA-1 | 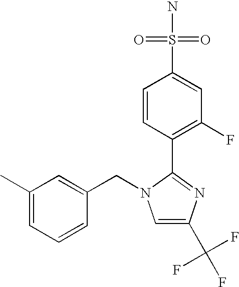 | 3-fluoro-4-[1-(3- methylbenzyl)- 4- (trifluoromethyl)- 1H-imidazol-2- yl]benzenesul- fonamide | 414 |
| AAA-1b | 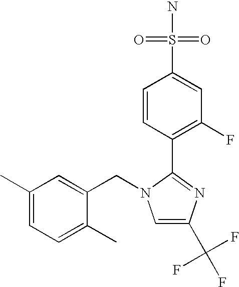 | 4-[1-(2,5- dimethylbenzyl)- 4- (trifluoromethyl)- 1H-imidazol-2- yl]-3- fluorobenzene- sulfonamide | 428 (M + 1) |
| | 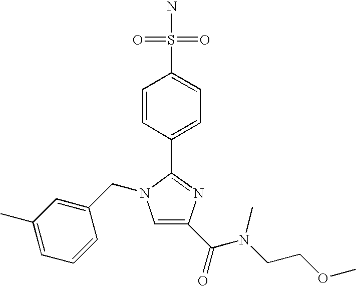 | 2-[4- (aminosulfonyl) phenyl]-N-(2- methoxyethyl)- N-methyl-1-(3- methylbenzyl)- 1H-imidazole-4- carboxamide | 443 (M + 1) |
| AAA-1c | 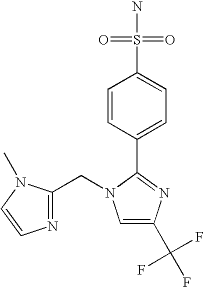 | 4-[1-[(1-methyl- 1H-imidazol-2- yl)methyl]-4- (trifluoromethyl)- 1H-imidazol-2- yl]benzenesul- fonamide | 386 (M + 1) |
| AAA-1d | 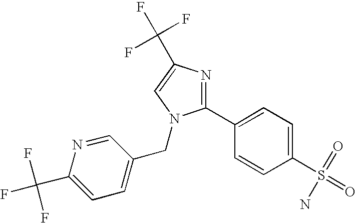 | 4-(4- (trifluoromethyl)- 1-{[6- (trifluoromethyl) pyridin-3- yl]methyl}-1H- imidazol-2- yl)benzenesul- fonamide | 451 (M + 1) |
| AAA-1e | 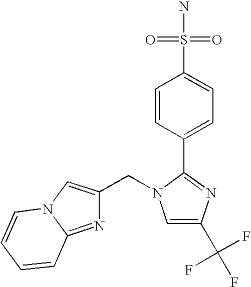 | 4-(1- (imidazo[1,2- b]pyridin-2- ylmethyl)-4- (trifluoromethyl)- 1H-imidazol-2- yl]benzenesul- fonamide | 422 (M + 1) |
| AAA-1f | 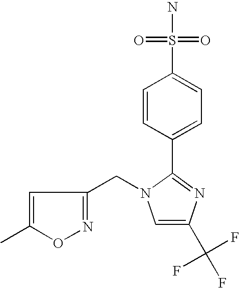 | 4-[1-[(5- methylisoxazol- 3-yl)methyl]-4- (trifluoromethyl)- 1H-imidazol-2- yl]benzenesul- fonamide | 387 (M + 1) |
| AAA-1g | 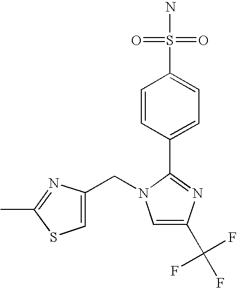 | 4-[1-[(2-methyl- 1,3-thiazol-4- yl)methyl]-4- (trifluoromethyl)- 1H-imidazol-2- yl]benzenesulfon- amide | 403 (M + 1) |
| AAA-1h | 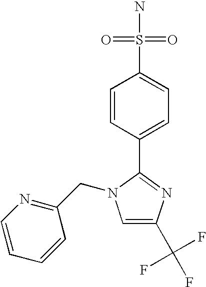 | 4-[1-(2-pyridin-2- ylmethyl)-4- (trifluoromethyl)- 1H-imidazol-2- yl]benzenesul- fonamide | 383 (M + 1) |
| AAA-1i | 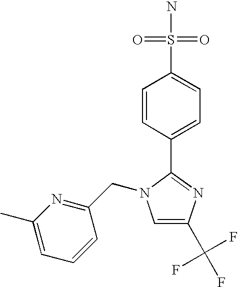 | 4-[1-[(6- methylpyridin-2- yl)methyl]-4- (trifluoromethyl)- 1H-imidazol-2- yl]benzenesul- fonamide | 397 (M + 1) |
| AAA-1j | 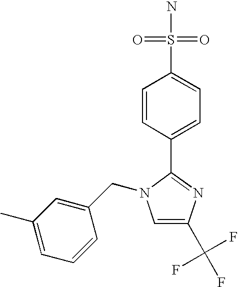 | 4-[1-(3- methylbenzyl)- 4- (trifluoromethyl)- 1H-imidazol-2- yl]benzenesul- fonamide | 396 (M + 1) |
| AAA-1k | 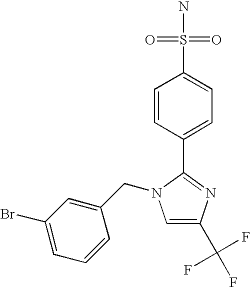 | 4-[1-(3- bromobenzyl)-4- (trifluoromethyl) 1H-imidazol-2- yl]benzenesul- fonamide | 460 (M + 1) |
| AAA-1l | 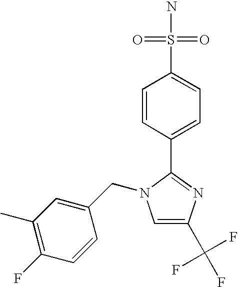 | 4-[1-(4-fluoro-3- methylbenzyl)- 4- (trifluoromethyl)- 1H-imidazol-2- yl]benzenesul- fonamide | 414 (M + 1) |
| CCC-1 | 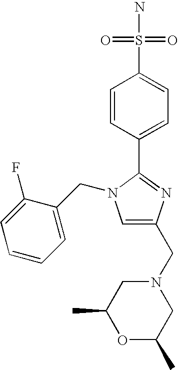 | 4-[4-{[(2R,6S)- 2,6- dimethylmorpholin- 4-yl]methyl}- 1-(2- fluorobenzyl)- 1H-imidazol-2- yl]benzenesul- fonamide | 459 (M + 1) |
| CCC-1a | 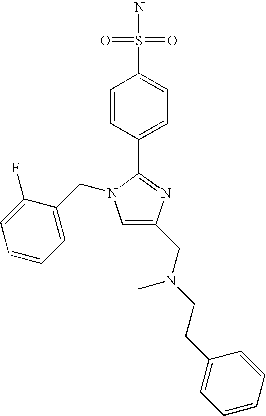 | 4-(1-(2- fluorobenzyl)-4- {[methyl(2- phenylethyl) amino]methyl}-1H- imidazol-2- yl)benzenesul- fonamide | 480 (M + 1) |
| CCC-1b | 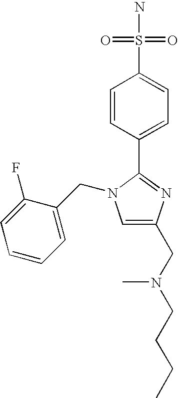 | 4-[4- {[butyl(methyl) amino]methyl}-1- (2-fluorobenzyl)- 1H-imidazol-2- yl]benzenesul- fonamide | 431 (M + 1) |
| AAA-1m | 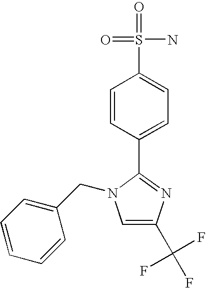 | 4-[1-benzyl-4- (trifluoromethyl)- 1H-imidazol-2- yl]benzenesul- fonamide | 382 (M + 1) |

| Example | | IC50_CAI | Kd_CAII | IC50_CAII | IC50_CAIV |
|---|---|---|---|---|---|
| number | 1H NMR | (uM) | (uM) | (uM) | (uM) |
| bbb-5 | ¹H NMR (300 | | | | |
| | MHz, DMSO-d6) | | | | |
| | 5.42 (s, 2 H) | | | | |
| | 7.02 (t, J = 7.54 | | | | |
| | Hz, 1 H) 7.13- | | | | |
| | 7.25 (m, 2 H) | | | | |
| | 7.32-7.41 (m, 2 | | | | |
| | H) 7.49 (br. s., 2 | | | | |
| | H) 7.87 (s, 4 H) | | | | |
| BBB-1 | ¹H NMR (300 | | | | |
| | MHz, DMSO-d6) | | | | |
| | 1.29 (t, J = 7.06 | | | | |
| | Hz, 3 H) 4.27 (q, | | | | |
| | J = 6.97 Hz, 2 H) | | | | |
| | 5.50 (s, 2 H) | | | | |
| | 7.02 (td, J = 7.68, | | | | |
| | 1.41 Hz, 1 H) | | | | |
| | 7.13-7.24 (m, 2 | | | | |
| | H) 7.32-7.43 | | | | |
| | (m, 1 H) 7.49 | | | | |
| | (br. s., 2 H) 7.64 | | | | |
| | (d, J = 8.20 Hz, 2 | | | | |
| | H) 7.91 (d, | | | | |
| | J = 6.20 Hz, 2 H) | | | | |
| | 8.06 (s, 1 H) | | | | |
| AAA-1a | 1H NMR (400 | 0.02 | 2.28 | 0 | 1.26 |
| | MHz, DMSO-D6) | | | | |
| | d 1.96 (s, 3 H), | | | | |
| | 2.14 (s, 3 H), | | | | |
| | 5.17 (s, 2 H), | | | | |
| | 6.56 (s, 1 H), | | | | |
| | 6.99 (t, J = 7.18 | | | | |
| | Hz, 2 H, 7.63 (s, | | | | |
| | 2 H), 7.67-7.80 | | | | |
| | (m, 3 H), 7.86- | | | | |
| | 7.99 (m, J = 1.01 | | | | |
| | Hz, 1 H) | | | | |
| AAA-1 | ¹H NMR (400 | 0.04 | 7.54 | | 0.79 |
| | MHz, acetone) δ | | | | |
| | 2.22 (s, 3H), | | | | |
| | 5.27 (s, 2H), | | | | |
| | 6.81 (s, 1H), | | | | |
| | 7.04 (s, 2H), | | | | |
| | 7.66 (s, 1H), | | | | |
| | 7.73-7.79 (m, | | | | |
| | 2H), 7.79-7.86 | | | | |
| | (m, 1H). | | | | |
| AAA-1b | 1H NMR (400 | | | | |
| | MHz, DMSO-D6) | | | | |
| | d 1.96 (s, 3 H), | | | | |
| | 2.14 (s, 3 H), | | | | |
| | 5.17 (s, 2 H), | | | | |
| | 6.56 (s, 1 H), | | | | |
| | 6.99 (t, J = 7.18 | | | | |
| | Hz, 2 H), 7.63 (s, | | | | |
| | 2 H), 7.67-7.80 | | | | |
| | (m, 3 H), 7.86- | | | | |
| | 7.99 (m, J = 1.01 | | | | |
| | Hz, 1 H) | | | | |
| | 1H NMR (400 | | | | |
| | MHz, MeOD) d | | | | |
| | 2.16-2.23 (m, 3 | | | | |
| | H), 3.38 (s, 1 H), | | | | |
| | 3.56 (t, J = 5.16 | | | | |
| | Hz, 1 H), 3.61 (s, | | | | |
| | 1 H), 4.02 (s, 1 | | | | |
| | H), 5.24 (s, 2 H), | | | | |
| | 6.77 (d, J = 7.55 | | | | |
| | Hz, 1 H), 6.81 (s, | | | | |
| | 1 H), 7.02 (d, | | | | |
| | J = 7.55 Hz, 1 H), | | | | |
| | 7.11 (t, J = 7.68 | | | | |
| | Hz, 1 H), 7.60- | | | | |
| | 7.67 (m, 3 H), | | | | |
| | 7.86 (d, J = 8.31 | | | | |
| | Hz, 2 H). | | | | |
| AAA-1c | 1H NMR (400 | | | | |
| | MHz, MeOD) d | | | | |
| | 3.65 (s, 3 H), | | | | |
| | 5.74 (s, 2 H), | | | | |
| | 7.41 (s, 1 H), | | | | |
| | 7.44-7.51 (m, | | | | |
| | J = 1.01 Hz, 1 H), | | | | |
| | 7.68 (d, J = 8.31 | | | | |
| | Hz, 2 H), 7.86 (s, | | | | |
| | 1 H), 7.96 (d, | | | | |
| | J = 8.31 Hz, 2 H). | | | | |
| AAA-1d | 1H NMR (400 | | | | |
| | MHz, MeOD) d | | | | |
| | 5.46 (s, 2 H), | | | | |
| | 7.50-7.60 (m, 1 | | | | |
| | H), 7.66 (t, | | | | |
| | J = 8.06 Hz, 3 H), | | | | |
| | 7.84 (s, 1 H), | | | | |
| | 7.88-7.95 (m, 2 | | | | |
| | H), 8.34 (s, 1 H). | | | | |
| AAA-1e | 1H NMR (400 | | | | |
| | MHz, MeOD) d | | | | |
| | 5.63 (s, 2 H), | | | | |
| | 7.40 (t, J = 6.92 | | | | |
| | Hz, 1 H), 7.70- | | | | |
| | 7.83 (m, J = 8.44, | | | | |
| | 8.44 Hz, 3 H), | | | | |
| | 7.86-7.97 (m, 4 | | | | |
| | H), 8.04 (s, 1 H), | | | | |
| | 8.64 (d, J = 6.80, | | | | |
| | 1.01 Hz, 1 H). | | | | |
| AAA-1f | 1H NMR (400 | | | | |
| | MHz, MeOD) d | | | | |
| | 2.31 (s, 3 H), | | | | |
| | 5.30 (s, 2 H), | | | | |
| | 5.98 (s, 1 H), | | | | |
| | 7.72 (d, J = 8.06 | | | | |
| | Hz, 2 H), 7.82 (s, | | | | |
| | 1 H), 7.95 (d, | | | | |
| | J = 8.06 Hz, 2 H). | | | | |
| AAA-1g | 1H NMR (400 | | | | |
| | MHz, MeOD) d | | | | |
| | 2.62 (s, 3 H), | | | | |
| | 5.38 (s, 2 H), | | | | |
| | 7.37 (s, 1 H), | | | | |
| | 7.82 (d, J = 8.06 | | | | |
| | Hz, 2 H), 7.97 | | | | |
| | (d, J = 8.31 Hz, 2 | | | | |
| | H), 8.02 (s, 1 H). | | | | |
| AAA-1h | 1H NMR (400 | | | | |
| | MHz, DMSO-D6) | | | | |
| | d 5.66 (s, 2 H), | | | | |
| | 7.27 (d, J = 7.81 | | | | |
| | Hz, 1 H), 7.39 | | | | |
| | (dd, J = 7.18, 5.16 | | | | |
| | Hz, 1 H), 7.48 (s, | | | | |
| | 2 H), 7.77-7.87 | | | | |
| | (m, 5 H), 8.10 (s, | | | | |
| | 1 H), 8.55 (d, | | | | |
| | J = 4.53 Hz, 1 H). | | | | |
| AAA-1i | 1H NMR (400 | | | 0 | 0.63 |
| | MHz, DMSO-D6) | | | | |
| | d 2.45-2.51 (m, | | | | |
| | J = 6.30 Hz, 3 H), | | | | |
| | 5.56 (s, 2 H), | | | | |
| | 7.05 (d, J = 7.55 | | | | |
| | Hz, 1 H), 7.37 | | | | |
| | (d, J = 7.55 Hz, 1 | | | | |
| | H), 7.49 (s, 2 H), | | | | |
| | 7.88 (s, 3 H), | | | | |
| | 8.11 (s, 1 H). | | | | |
| AAA-1j | 1H NMR (400 | 0.1 | | 0 | 0.33 |
| | MHz, MeOD) d | | | | |
| | 2.23 (s, 3 H), | | | | |
| | 5.33 (s, 2 H), | | | | |
| | 6.85 (d, J = 7.81 | | | | |
| | Hz, 1 H), 6.89 (s, | | | | |
| | 1 H), 7.12 (d, 1 | | | | |
| | H), 7.21 (t, | | | | |
| | J = 7.68 Hz, 1 H), | | | | |
| | 7.74 (d, J = 8.31 | | | | |
| | Hz, 2 H), 7.90 (s, | | | | |
| | 1 H), 8.01 (d, | | | | |
| | J = 8.06 Hz, 2 H). | | | | |
| AAA-1k | 1H NMR (400 | 0.08 | | 0 | 0.31 |
| | MHz, MeOD) d | | | | |
| | 5.29 (s, 2 H), | | | | |
| | 6.90 (d, J = 7.81 | | | | |
| | Hz, 1 H), 7.10- | | | | |
| | 7.18 (m, 2 H), | | | | |
| | 7.37 (d, J = 8.06 | | | | |
| | Hz, 1 H), 7.63 | | | | |
| | (d, J = 8.31 Hz, 2 | | | | |
| | H), 7.76 (s, 1 H), | | | | |
| | 7.91 (d, J = 8.31 | | | | |
| | Hz, 2 H). | | | | |
| AAA-1l | 1H NMR (400 | 0.05 | 2.45 | 0 | 0.59 |
| | MHz, MeOD) d | | | | |
| | 2.11 (s, 3 H), | | | | |
| | 5.21 (s, 2 H), | | | | |
| | 6.73-6.81 (m, 1 | | | | |
| | H), 6.82-6.96 | | | | |
| | (m, 2 H), 7.63 (d, | | | | |
| | J = 8.31 Hz, 2 H), | | | | |
| | 7.71 (s, 1 H), | | | | |
| | 7.91 (d,J = 8.31 | | | | |
| | Hz, 2 H). | | | | |
| CCC-1 | 1H NMR (400 | 0.03 | 2.38 | 0 | 0.21 |
| | MHz, MeOD) d | | | | |
| | 1,11 (d, J = 6.30 | | | | |
| | Hz, 6 H), 2.82 | | | | |
| | (d, J = 11.08 Hz, | | | | |
| | 2 H), 3.52 (s, 2 | | | | |
| | H), 3.58-3.72 | | | | |
| | (m, 2 H), 5.17- | | | | |
| | 5.41 (m, 2 H), | | | | |
| | 6.99 (t, J = 7.05 | | | | |
| | Hz, 1 H), 7.04- | | | | |
| | 7.14 (m, 2 H), | | | | |
| | 7.17 (s, 1 H), | | | | |
| | 7.28-7.37 (m, 1 | | | | |
| | H), 7.71 (d, | | | | |
| | J = 8.31 Hz, 2 H), | | | | |
| | 7.98 (d, J = 8.31 | | | | |
| | Hz, 2 H). | | | | |
| CCC-1a | 1H NMR (400 | 0.05 | 2.5 | 0 | 0.96 |
| | MHz, MeOD) d | | | | |
| | 2.40 (s, 3 H), | | | | |
| | 2.62-2.72 (m, 2 | | | | |
| | H), 2.79-2.88 | | | | |
| | (m, 2 H), 3.67 (s, | | | | |
| | 2 H), 5.36 (s, 2 | | | | |
| | H), 7.00 (t, | | | | |
| | J = 6.92 Hz, 1 H), | | | | |
| | 7.06-7.21 (m, 5 | | | | |
| | H), 7.22-7.29 | | | | |
| | (m, 2 H), 7.31- | | | | |
| | 7.40 (m, 1 H), | | | | |
| | 7.74 (d, J = 8.81 | | | | |
| | Hz, 2 H), 8.00 | | | | |
| | (d, J = 8.81 Hz, 2 | | | | |
| | H). | | | | |
| CCC-1b | 1H NMR (400 | 0.08 | 3.37 | | 1.14 |
| | MHz, MeOD) d | | | | |
| | 0.83 (t, J = 7.43 | | | | |
| | Hz, 3 H), 1.17- | | | | |
| | 1.28 (m, 2 H), | | | | |
| | 1.35-1.51 (m, 2 | | | | |
| | H), 2.28-2.40 | | | | |
| | (m, 2 H), 3.50 (s, | | | | |
| | 2 H), 5.26 (s, 2 | | | | |
| | H), 6.87-6.94 | | | | |
| | (m, 1 H), 6.95- | | | | |
| | 7.06 (m, 2 H), | | | | |
| | 7.09 (s, 1 H), | | | | |
| | 7.19-7.29 (m, 1 | | | | |
| | H), 7.63 (d, | | | | |
| | J = 8.31 Hz, 2 H), | | | | |
| | 7.90 (d, J = 6.31 | | | | |
| | Hz, 2 H). | | | | |
| AAA-1m | 1H NMR (400 | | | | |
| | MHz, MeOD) d | | | | |
| | 5.27 (s, 2 H), | | | | |
| | 6.96 (d, J = 7.55 | | | | |
| | Hz, 2 H), 7.15- | | | | |
| | 7.28 (m, J = 7.55 | | | | |
| | Hz, 3 H), 7.63 | | | | |
| | (d, J = 7.81 Hz, 2 | | | | |
| | H), 7.72 (s, 1 H), | | | | |
| | 7.89 (d, J = 7.81 | | | | |
| | Hz, 2 H). | | | | |

| | | Observed | Exact | |
|---|---|---|---|---|
| Example # | Structure | Mass | MW | Synthetic Method |
| R-1 | 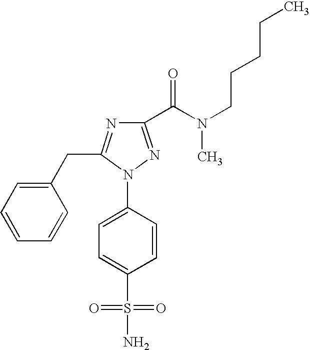 | 441.8 | 441.2 | L A |
| R-2 | 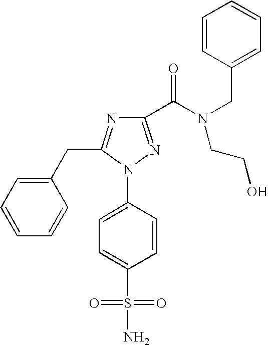 | 492.5 | 491.2 | L A |
| R-3 | 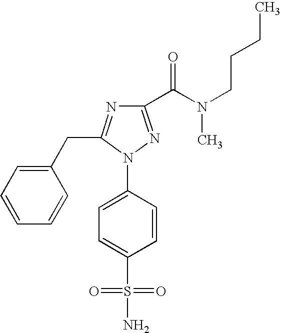 | 428.0 | 427.2 | L A |
| R-4 | 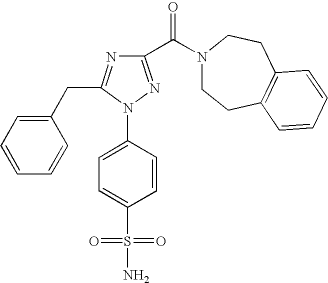 | 488.3 | 487.2 | L A |
| R-5 | 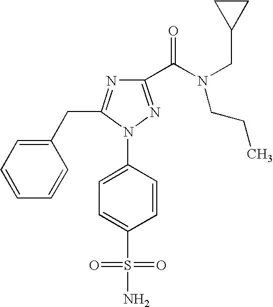 | 454.0 | 453.2 | L A |
| R-6 | 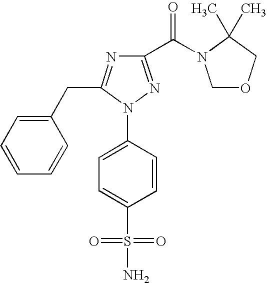 | 442.3 | 441.2 | L A |
| R-7 | 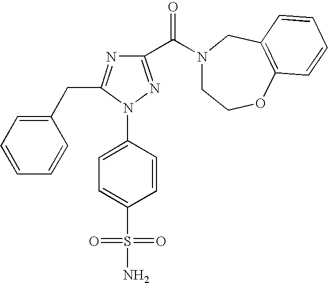 | 490.0 | 489.2 | L A |
| R-8 | 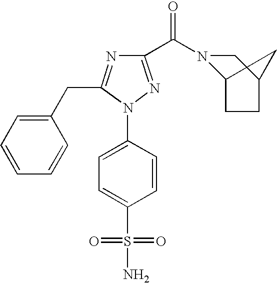 | 438.3 | 437.2 | L A |
| R-9 | 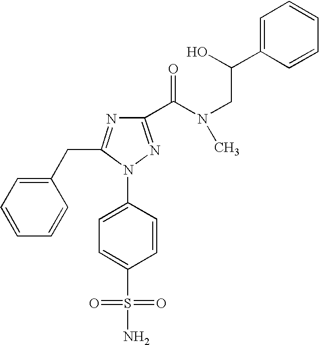 | 492.5 | 491.2 | L A |
| R-10 | 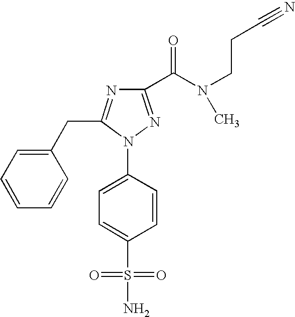 | 425.0 | 424.1 | L A |
| R-11 | 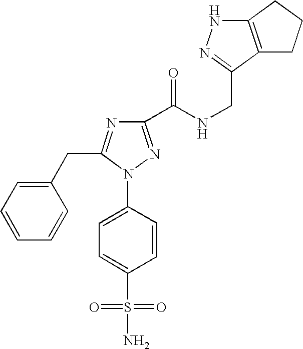 | 478.3 | 477.2 | L A |
| R-12 | 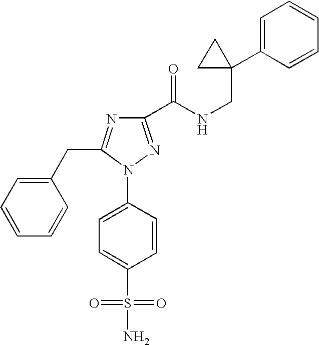 | 488.0 | 487.2 | L A |
| R-13 | 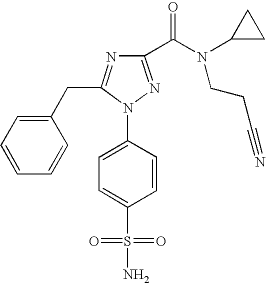 | 451.0 | 450.2 | L A |
| R-14 | 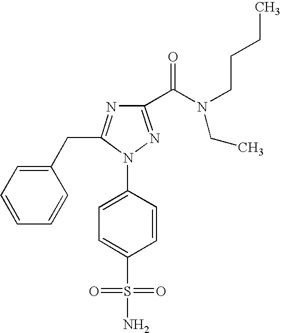 | 442.3 | 441.2 | L A |
| R-15 | 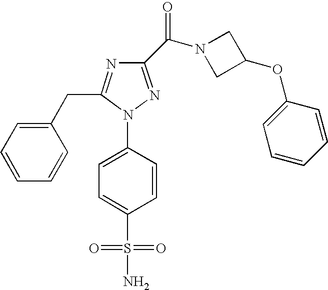 | 490.4 | 489.2 | L A |
| R-16 | 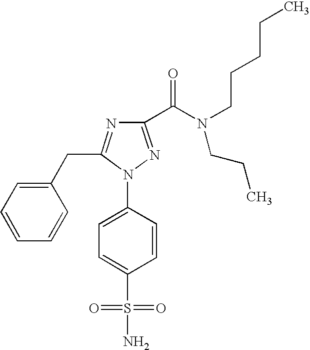 | 472.4 | 471.2 | L A |
| R-17 | 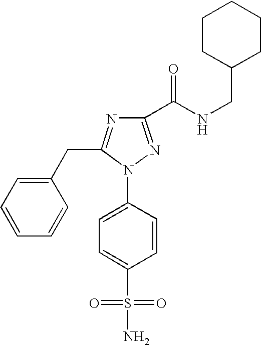 | 454.0 | 453.2 | L A |
| R-18 | 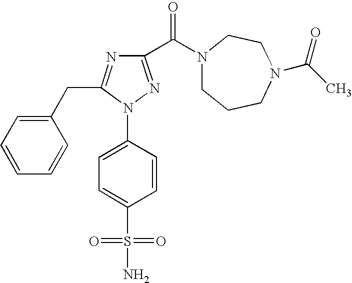 | 482.9 | 482.2 | L A |
| R-19 | 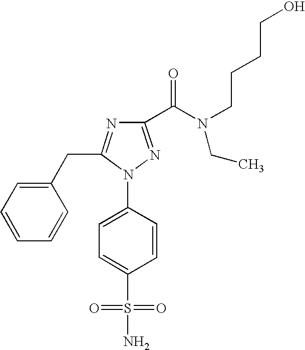 | 458.1 | 457.2 | L A |
| R-20 | 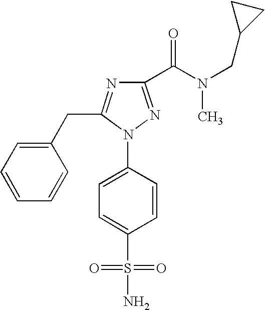 | 426.5 | 425.2 | L A |
| R-21 | 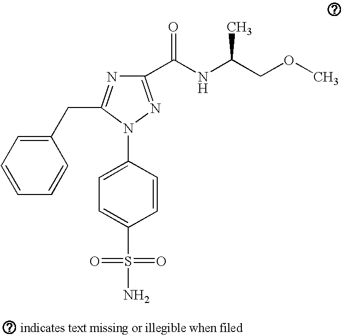 | 429.8 | 429.2 | L A |
| R-22 | 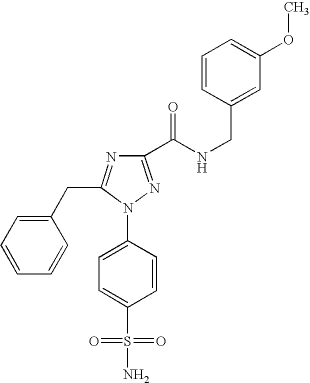 | 478.3 | 477.2 | L A |
| R-23 | 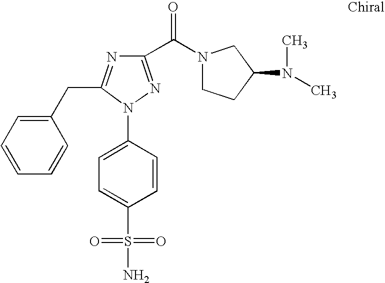 | 455.1 | 454.2 | L A |
| R-24 | 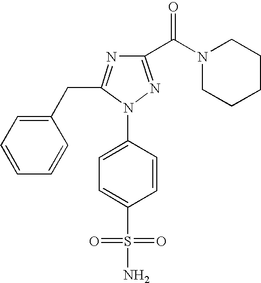 | 426.3 | 425.2 | L A |
| R-25 | 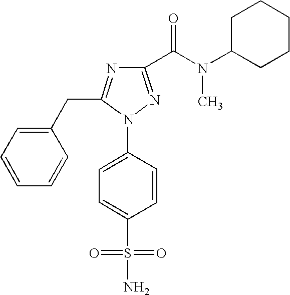 | 454.1 | 453.2 | L A |
| R-26 | 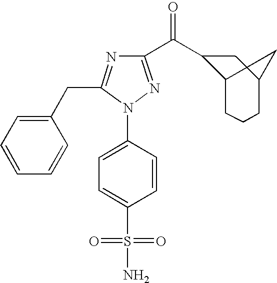 | 452.4 | 451.2 | L A |
| R-27 | 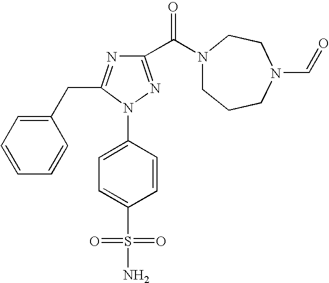 | 469.5 | 468.2 | L A |
| R-28 | 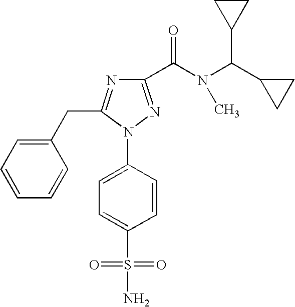 | 466.4 | 465.2 | L A |
| R-29 | 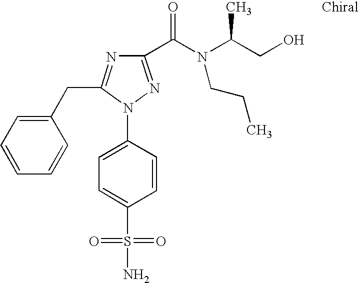 | 458.4 | 457.2 | L A |
| R-30 | 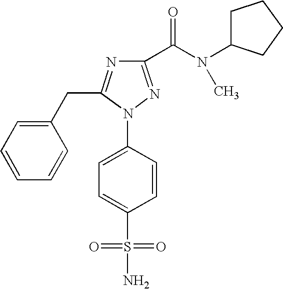 | 440.0 | 439.2 | L A |
| R-31 | 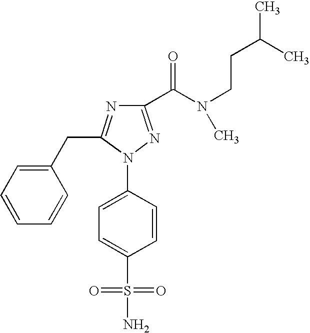 | 442.5 | 441.2 | L A |
| R-32 | 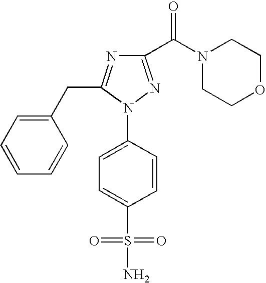 | 428.0 | 427.1 | L A |
| R-33 | 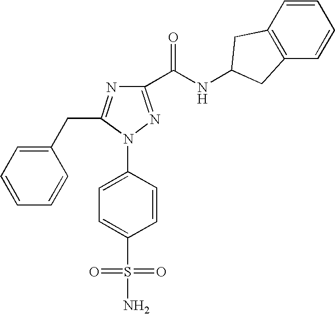 | 474.4 | 473.2 | L A |
| R-34 | 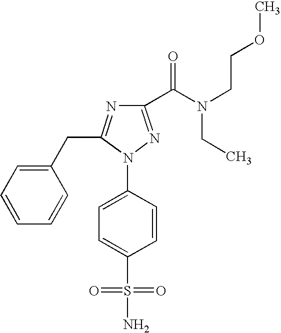 | 444.1 | 443.2 | L A |
| R-35 | 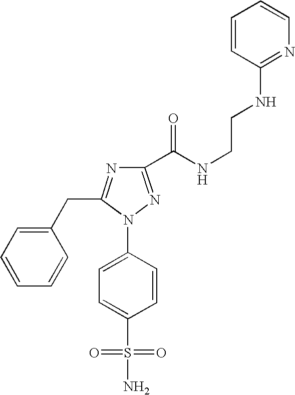 | 478.0 | 477.2 | L A |
| R-36 | 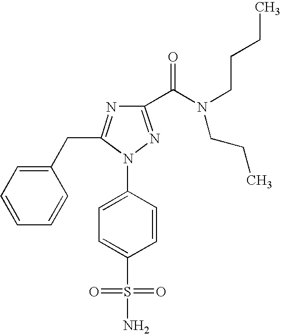 | 456.3 | 455.2 | L A |
| R-37 | 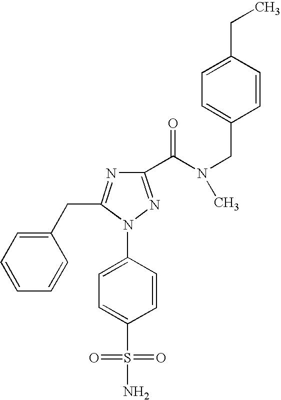 | 490.4 | 489.2 | L A |
| R-38 | 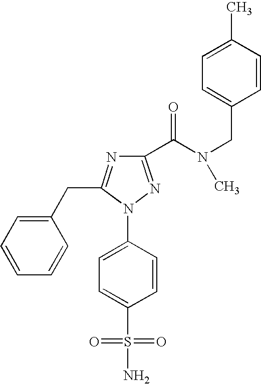 | 476.3 | 475.2 | L A |
| R-39 | 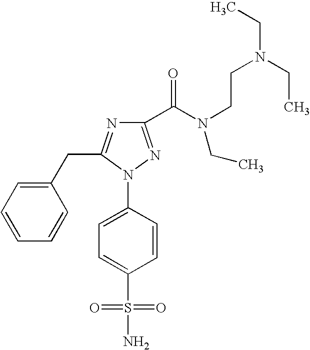 | 485.5 | 484.2 | L A |
| R-40 | 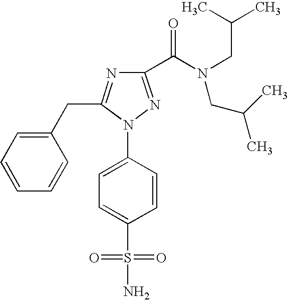 | 470.6 | 469.2 | L A |
| R-41 | 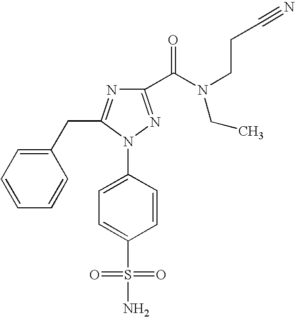 | 439.3 | 438.2 | L A |
| R-42 | 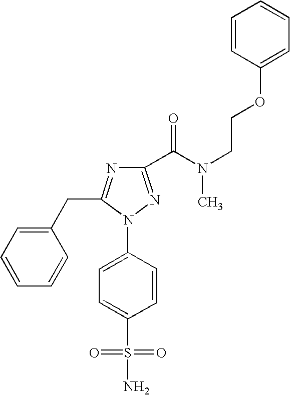 | 492.5 | 491.2 | L A |
| R-43 | 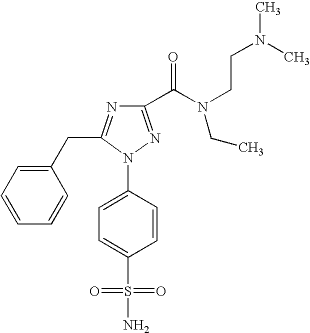 | 457.5 | 456.2 | L A |
| R-44 | 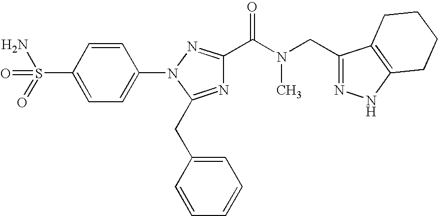 | 506.2 | 505.2 | L A |
| R-45 | 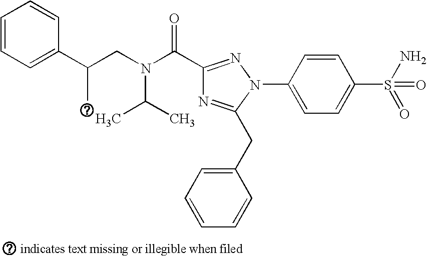 | 520.2 | 519.2 | L A |
| R-46 | 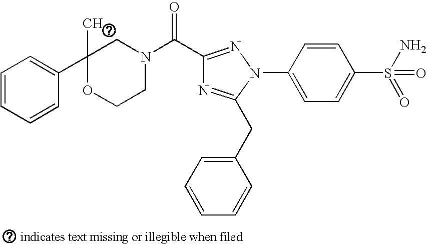 | 518.2 | 517.2 | L A |
| R-47 | 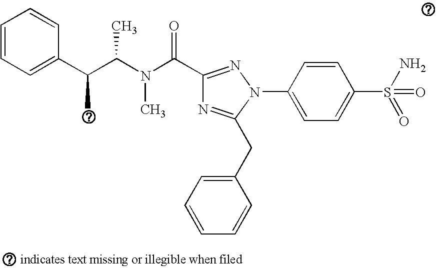 | 506.2 | 505.2 | L A |
| R-48 | 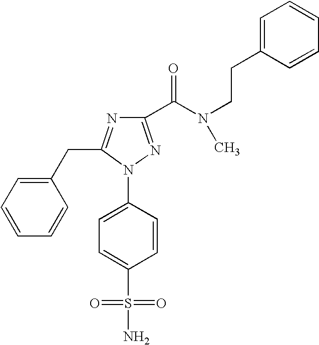 | 476.2 | 475.2 | L A |
| R-49 | 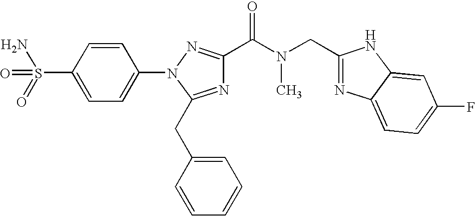 | 520.1 | 519.1 | L A |
| R-50 | 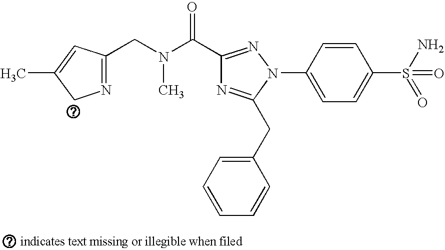 | 466.2 | 465.2 | L A |
| R-51 | 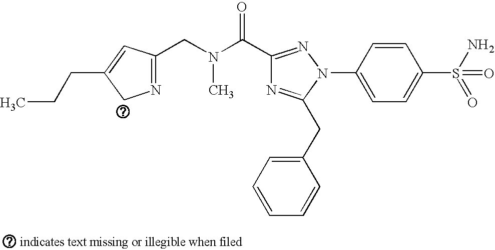 | 494.2 | 493.2 | L A |
| R-52 | 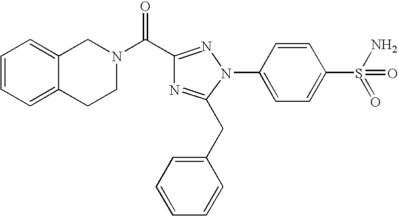 | 474.2 | 473.2 | L A |
| R-53 | 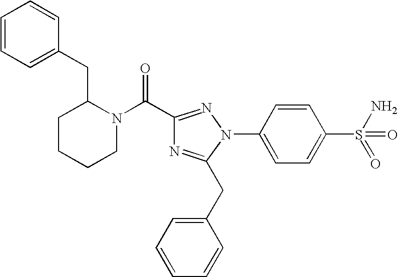 | 516.2 | 515.2 | L A |
| R-54 | 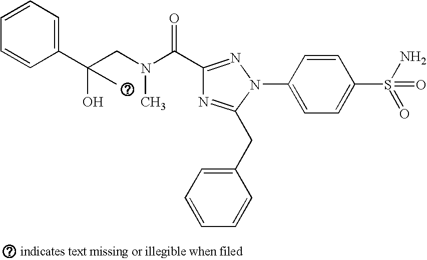 | 506.2 | 505.2 | L A |
| R-55 | 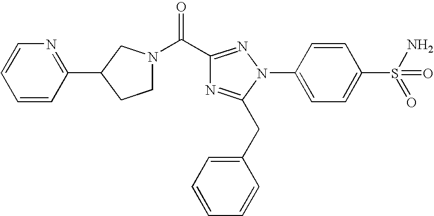 | 489.2 | 488.2 | L A |
| R-56 | 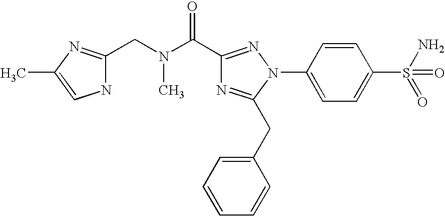 | 483.1 | 482.1 | L A |
| R-57 | 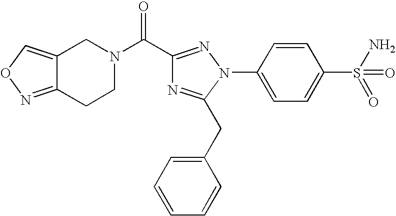 | 465.1 | 464.1 | L A |
| R-58 | 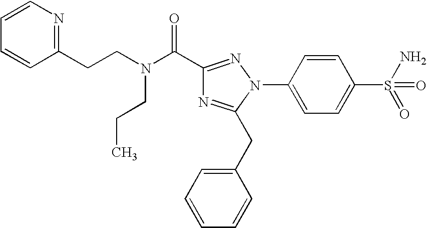 | 505.2 | 504.2 | L A |
| R-59 | 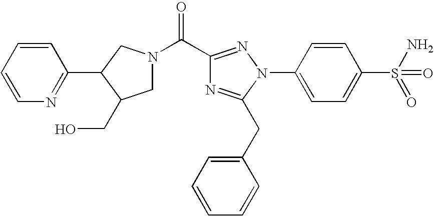 | 519.2 | 518.2 | L A |
| R-60 | 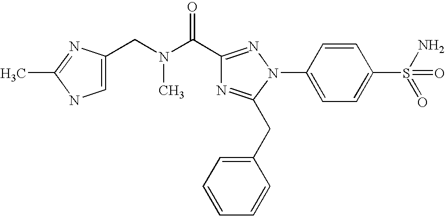 | 483.1 | 482.1 | L A |
| R-61 | 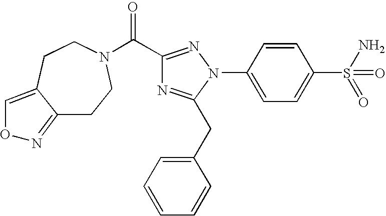 | 479.1 | 478.1 | L A |
| R-62 | 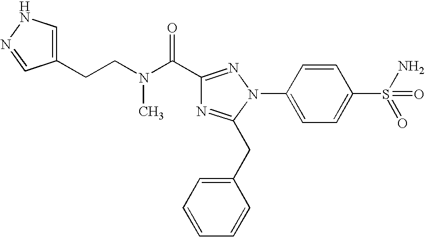 | 466.2 | 465.2 | L A |
| R-63 | 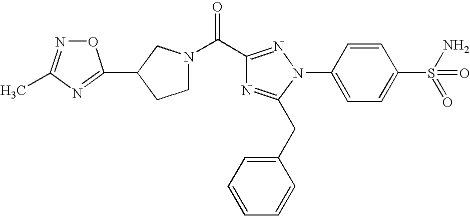 | 494.2 | 493.2 | L A |
| R-64 | 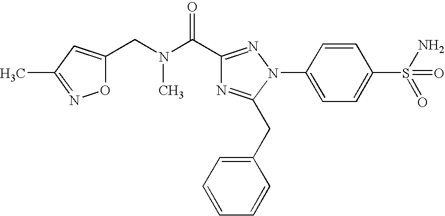 | 467.1 | 466.1 | L A |
| R-65 | 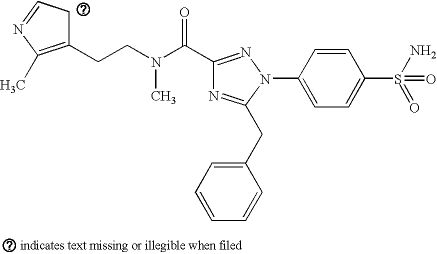 | 497.1 | 496.1 | L A |
| R-66 | 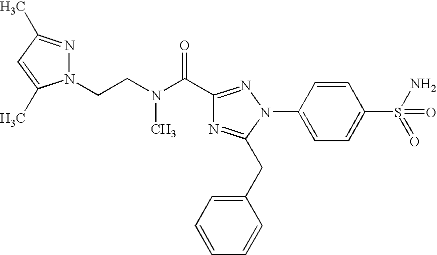 | 494.2 | 493.2 | L A |
| R-67 | 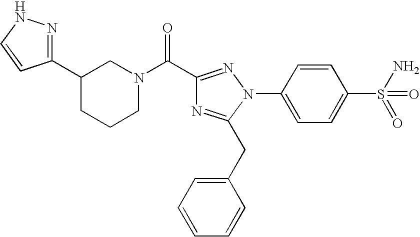 | 492.2 | 491.2 | L A |
| R-68 | 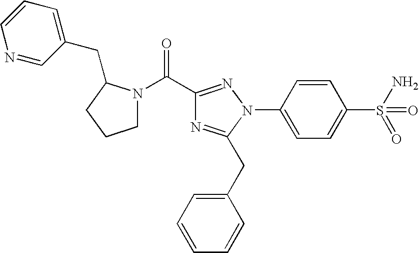 | 503.2 | 502.2 | L A |
| R-69 | 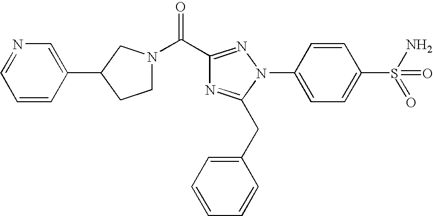 | 489.2 | 488.2 | L A |
| R-70 | 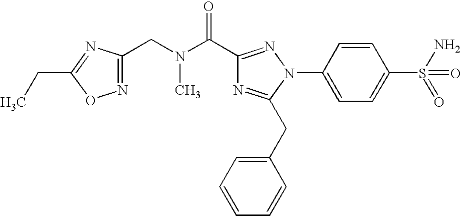 | 482.2 | 481.2 | L A |
| R-71 | 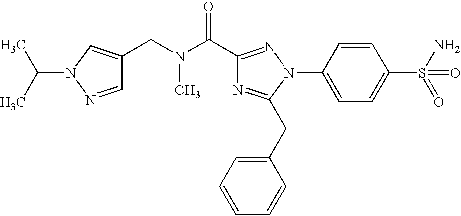 | 494.2 | 493.2 | L A |
| R-72 | 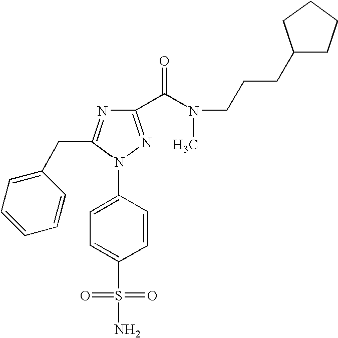 | 482.3 | 481.2 | L A |
| R-73 | 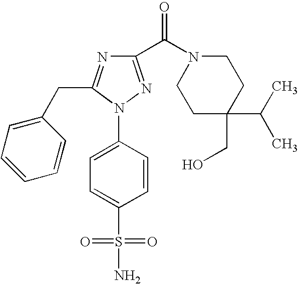 | 498.6 | 497.2 | L A |
| R-74 | 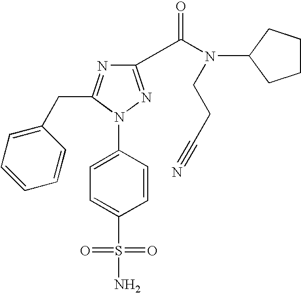 | 479.5 | 478.2 | L A |
| R-75 | 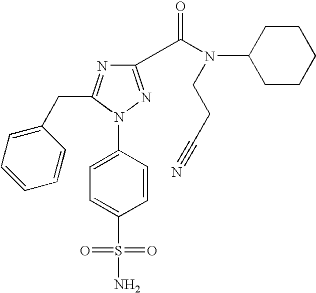 | 493.5 | 492.2 | L A |
| R-76 | 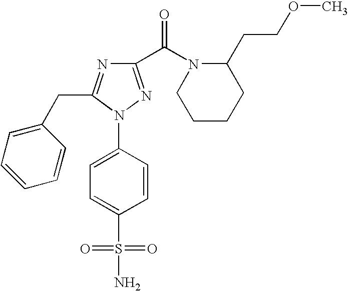 | 484.4 | 483.2 | L A |
| R-77 | 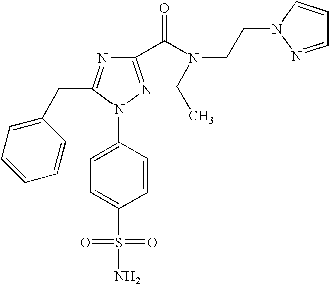 | 480.4 | 479.2 | L A |
| R-78 | 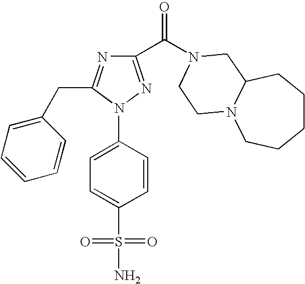 | 495.5 | 494.2 | L A |
| R-79 | 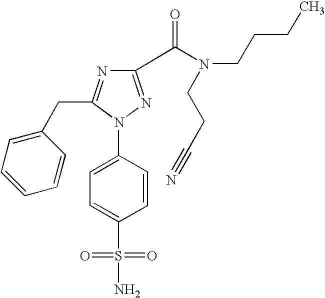 | 467.3 | 466.2 | L A |
| R-80 | 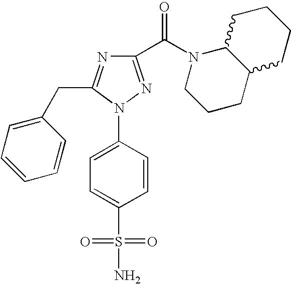 | 480.4 | 479.2 | L A |
| R-81 | 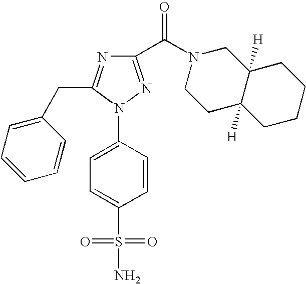 | 480.5 | 479.2 | L A |
| R-82 | 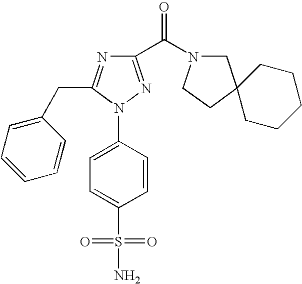 | 480.4 | 479.2 | L A |
| R-83 | 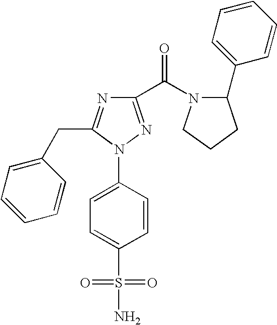 | 488.4 | 487.2 | L A |
| R-84 | 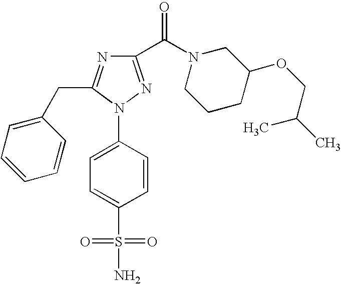 | 498.5 | 497.2 | L A |
| R-85 | 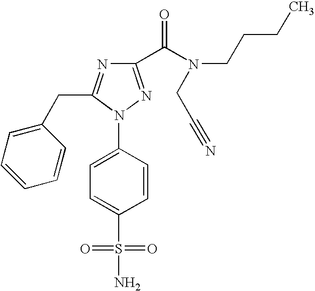 | 453.4 | 452.2 | L A |
| R-86 | 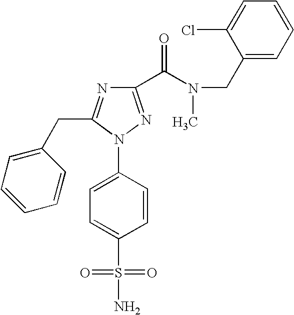 | 496.4 | 495.1 | L A |
| R-87 | 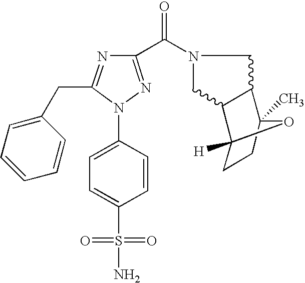 | 494.6 | 493.2 | L A |
| R-88 | 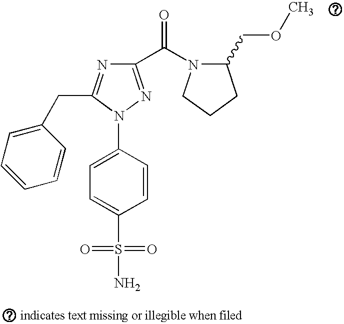 | 456.4 | 455.2 | L A |
| R-89 | 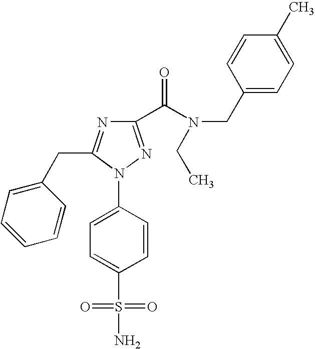 | 490.5 | 489.2 | L A |
| R-90 | 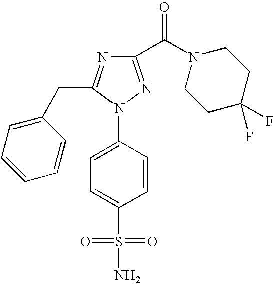 | 462.5 | 461.1 | L A |
| R-91 | 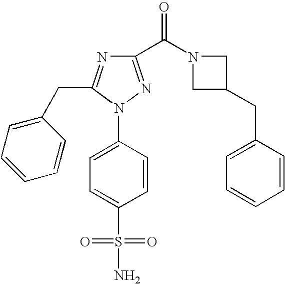 | 488.4 | 487.2 | L A |
| R-92 | 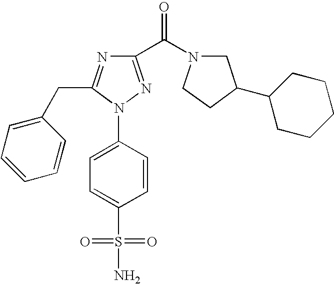 | 494.8 | 493.2 | L A |
| R-93 | 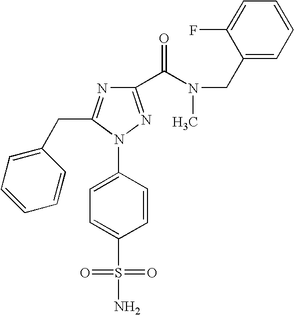 | 480.4 | 479.1 | L A |
| R-94 | 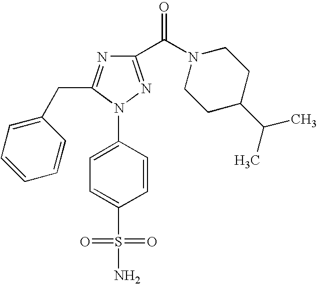 | 468.6 | 467.2 | L A |
| R-95 | 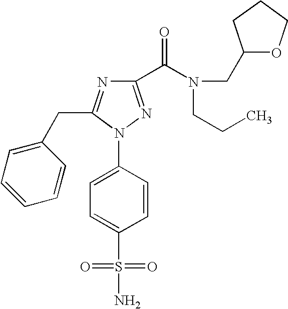 | 484.5 | 483.2 | L A |
| R-96 | 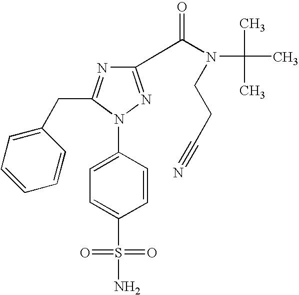 | 467.3 | 466.2 | L A |
| R-97 | 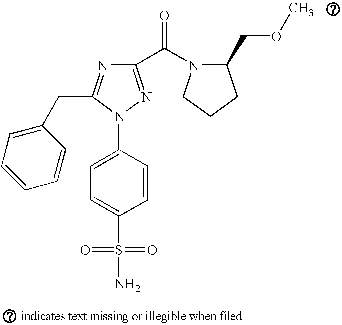 | 456.5 | 455.2 | L A |
| R-98 | 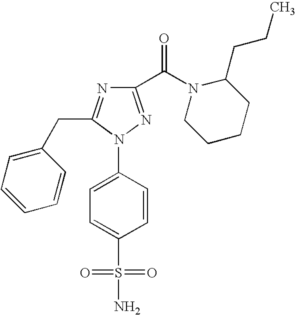 | 468.6 | 467.2 | L A |
| R-99 | 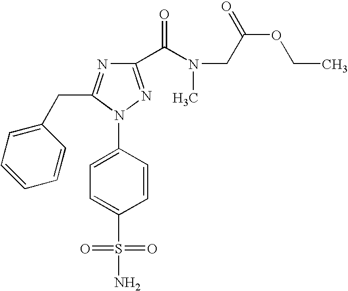 | 458.4 | 457.1 | L A |
| R-100 | 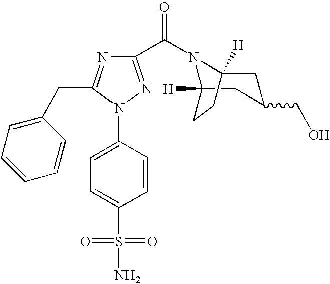 | 482.5 | 481.2 | L A |
| R-101 | 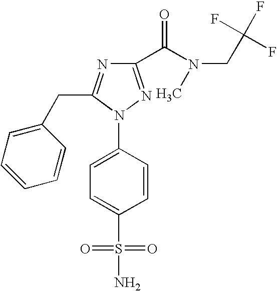 | 454.3 | 453.1 | L A |
| R-102 | 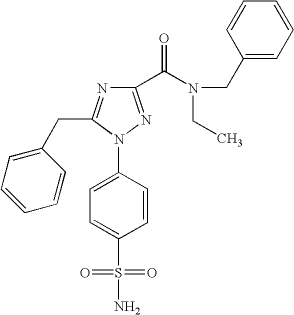 | 476.5 | 475.2 | L A |
| R-103 | 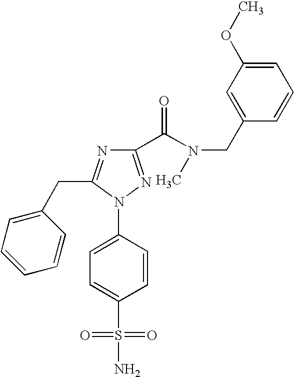 | 492.4 | 491.2 | L A |
| R-104 | 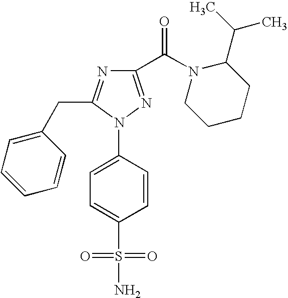 | 468.5 | 467.2 | L A |
| R-105 | 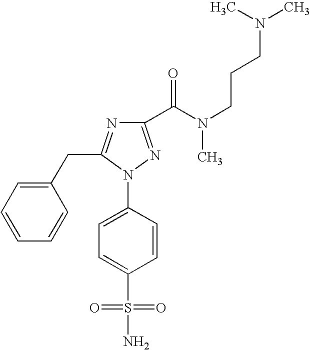 | 457.5 | 456.2 | L A |
| R-106 | 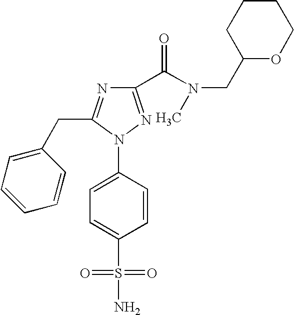 | 470.6 | 469.2 | L A |
| R-107 | 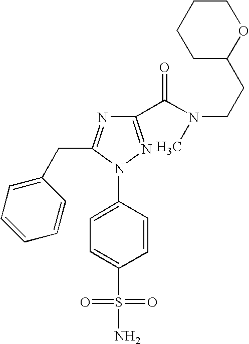 | 484.5 | 483.2 | L A |
| R-108 | 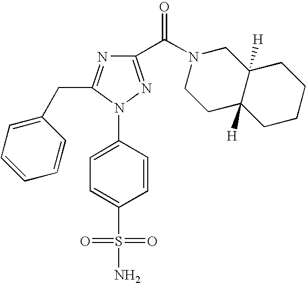 | 480.4 | 479.2 | L A |
| R-109 | 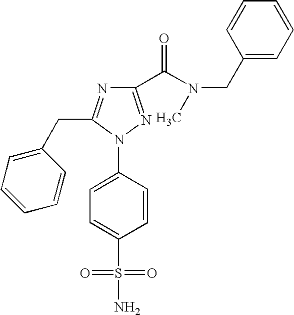 | 462.5 | 461.2 | L A |
| R-110 | 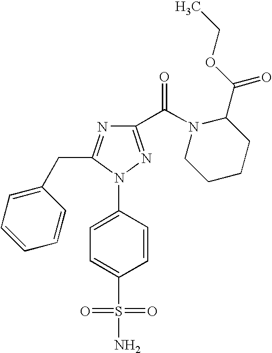 | 498.5 | 497.2 | L A |
| R-111 | 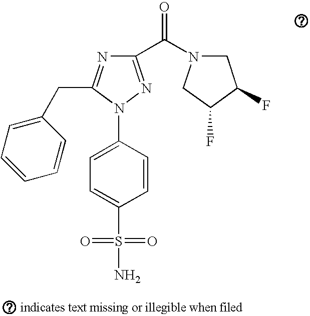 | 448.5 | 447.1 | L A |
| R-112 | 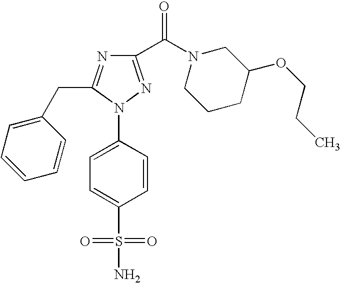 | 484.5 | 483.2 | L A |
| R-113 | 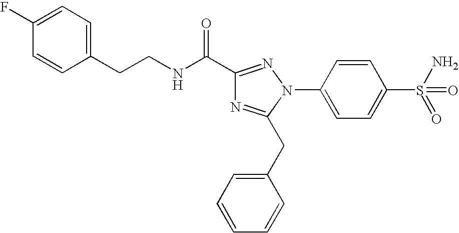 | 480.1 | 479.1 | L A |
| R-114 | 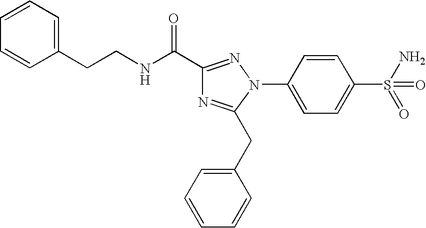 | 462.2 | 461.2 | L A |
| R-115 | 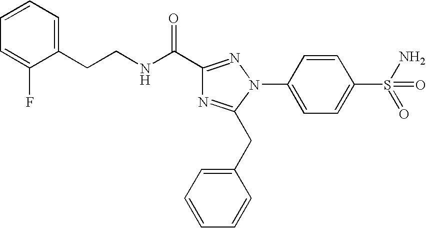 | 480.1 | 479.1 | L A |
| R-116 | 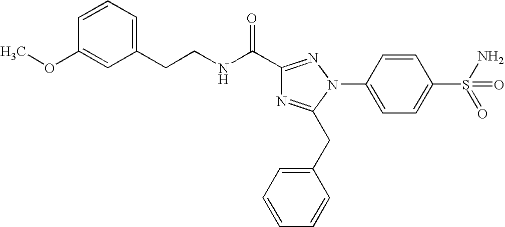 | 492.2 | 491.2 | L A |
| R-117 | 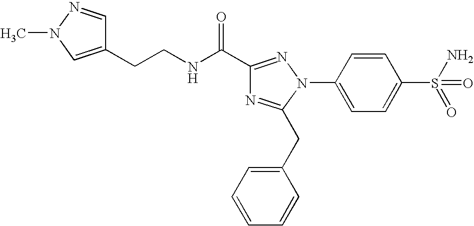 | 466.2 | 465.2 | L A |
| R-118 | 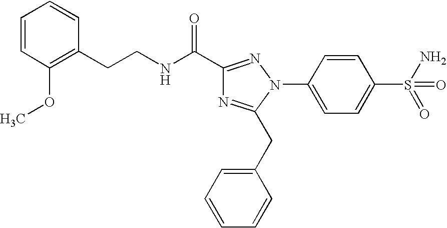 | 492.2 | 491.2 | L A |
| R-119 | 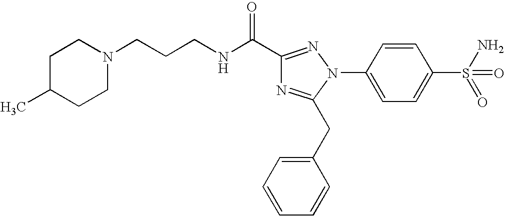 | 497.2 | 496.2 | L A |
| R-120 | 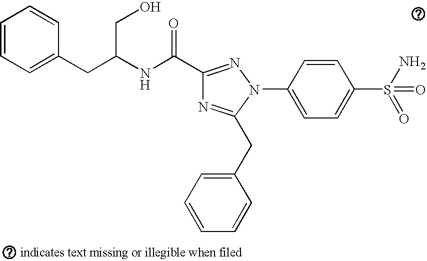 | 492.2 | 491.2 | L A |
| R-121 | 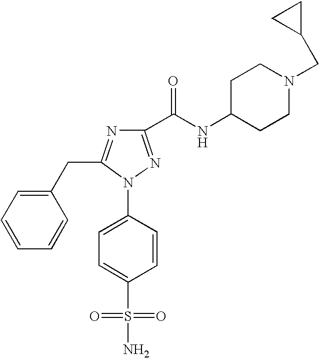 | 495.2 | 494.2 | L A |
| R-122 | 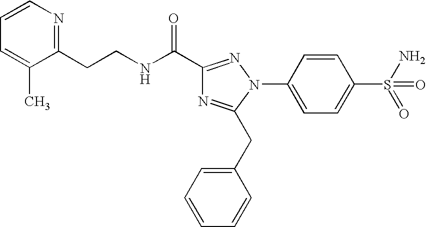 | 477.2 | 476.2 | L A |
| S-1 | 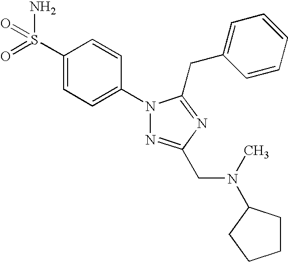 | 426.2 | 425.2 | L B |
| S-2 | 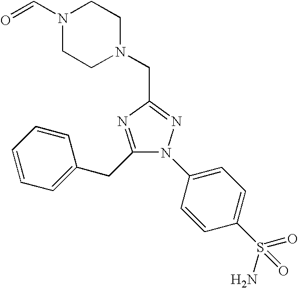 | 441.2 | 440.2 | L B |
| S-3 | 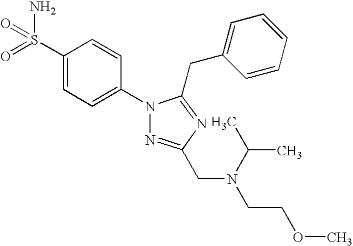 | 444.2 | 443.2 | L B |
| S-4 | 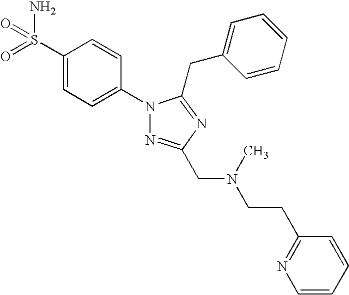 | 463.2 | 462.2 | L B |
| S-5 | 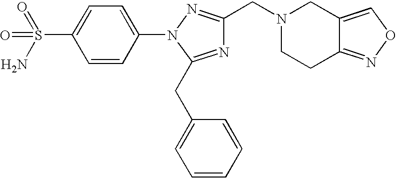 | 451.1 | 450.1 | L B |
| S-6 | 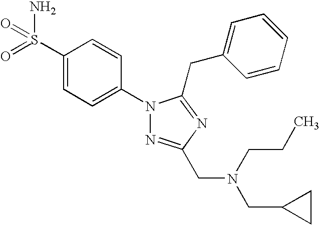 | 440.2 | 439.2 | L B |
| S-7 | 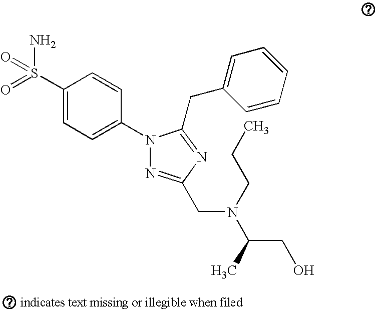 | 444.2 | 443.2 | L B |
| S-8 | 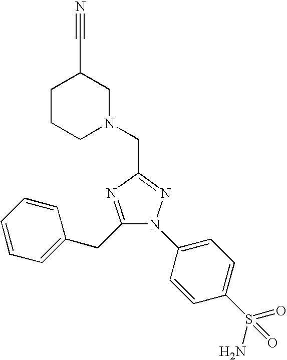 | 437.2 | 436.2 | L B |
| S-9 | 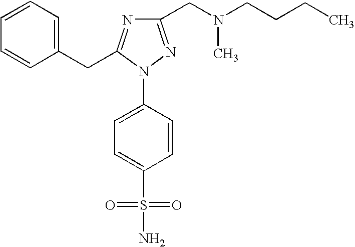 | 414.2 | 413.2 | L B |
| S-10 | 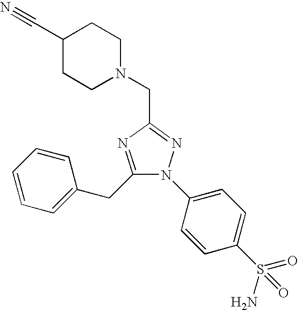 | 437.2 | 436.2 | L B |
| S-11 | 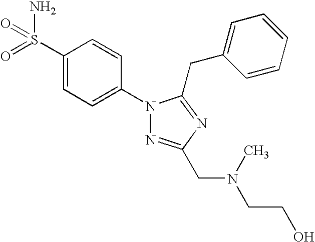 | 402.2 | 401.2 | L B |
| S-12 | 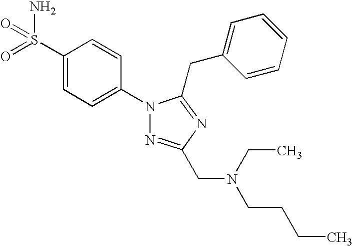 | 428.2 | 427.2 | L B |
| S-13 | 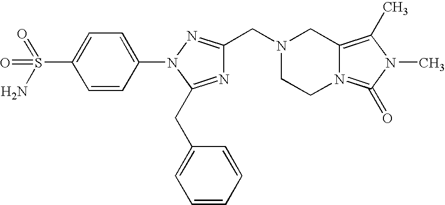 | 494.2 | 493.2 | L B |
| S-14 | 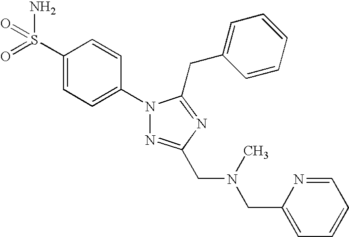 | 449.2 | 448.2 | L B |
| S-15 | 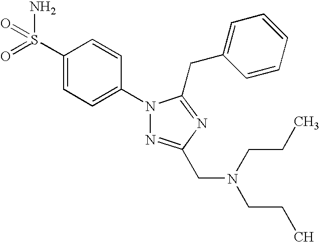 | 430.2 | 429.2 | L B |
| S-16 | 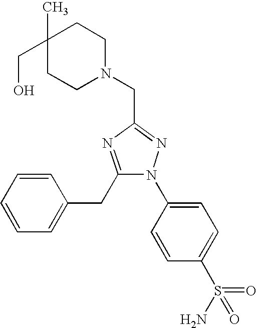 | 456.2 | 455.2 | L B |
| S-17 | 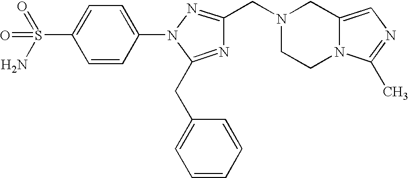 | 464.2 | 463.2 | L B |
| S-18 | 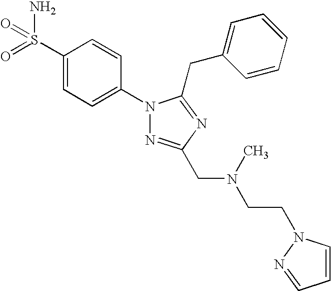 | 452.2 | 451.2 | L B |
| S-19 | 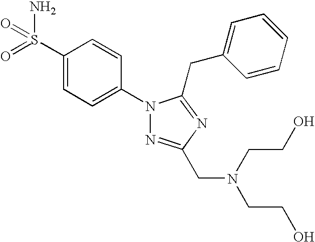 | 432.2 | 431.2 | L B |
| S-20 | 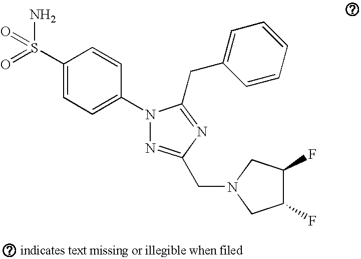 | 434.1 | 433.1 | L B |
| S-21 | 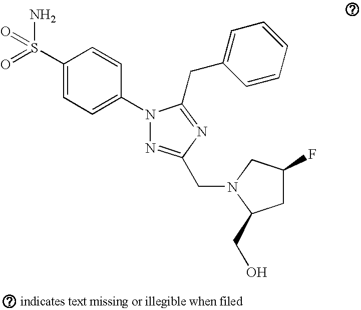 | 446.2 | 445.2 | L B |
| S-22 | 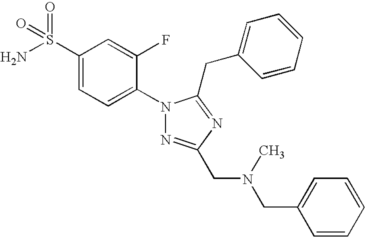 | 466 | 465.2 | L B |
| S-23 | 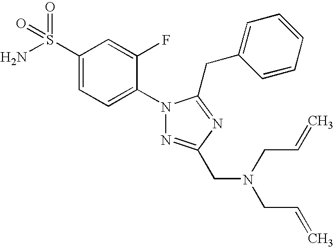 | 442 | 441.2 | L B |
| S-24 | 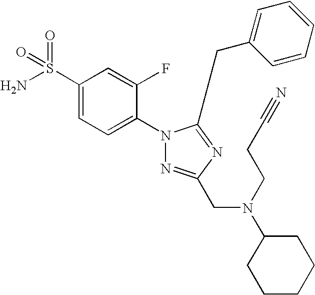 | 497 | 496.2 | L B |
| S-25 | 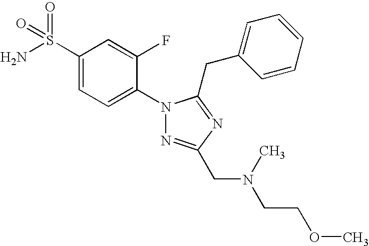 | 434 | 433.2 | L B |
| S-26 | 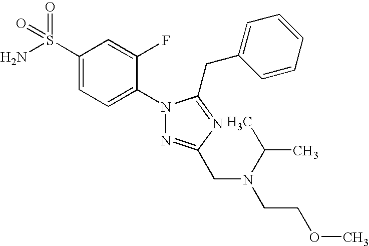 | 462 | 461.2 | L B |
| S-27 | 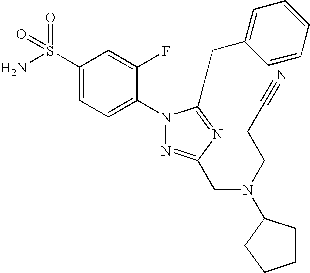 | 483 | 482.2 | L B |
| S-28 | 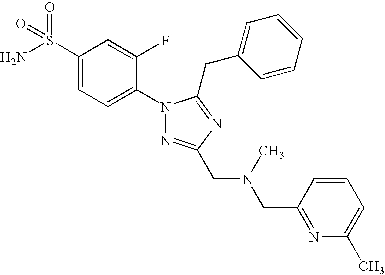 | 481 | 480.2 | L B |
| S-29 | 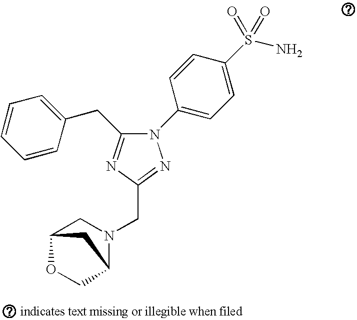 | 444 | 443.1 | L B |
| S-30 | 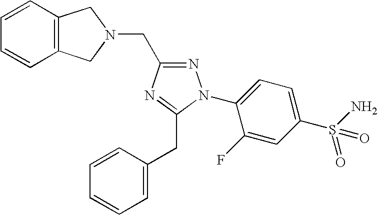 | 464 | 463.1 | L B |
| S-31 | 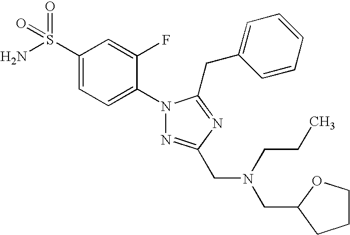 | 488 | 487.2 | L B |
| S-32 | 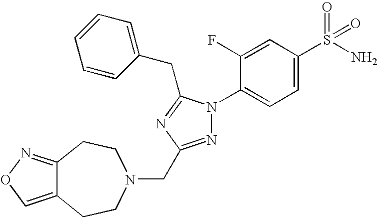 | 483 | 482.2 | L B |
| S-33 | 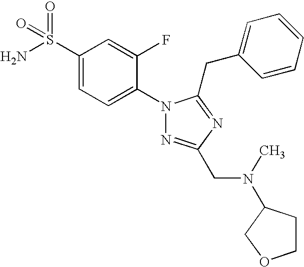 | 446 | 445.2 | L B |
| S-34 | 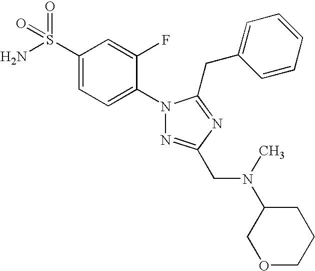 | 460 | 459.2 | L B |
| S-35 | 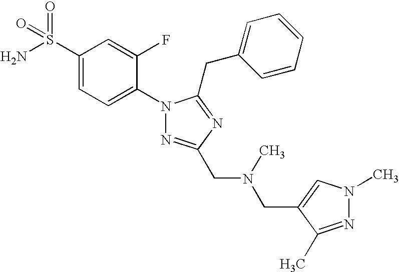 | 484 | 483.2 | L B |
| S-36 | 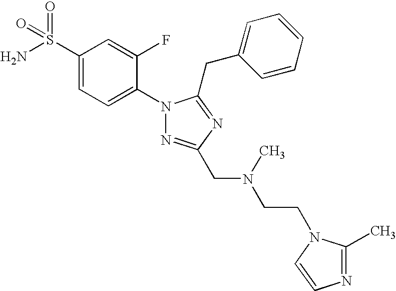 | 484 | 483.2 | L B |
| S-37 | 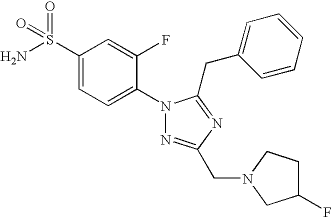 | 434 | 433.1 | L B |
| S-38 | 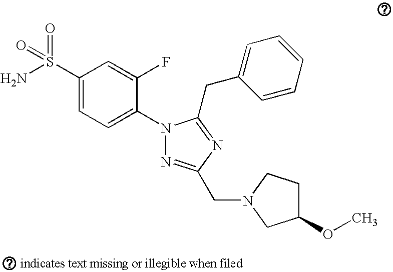 | 446 | 445.2 | L B |
| S-39 | 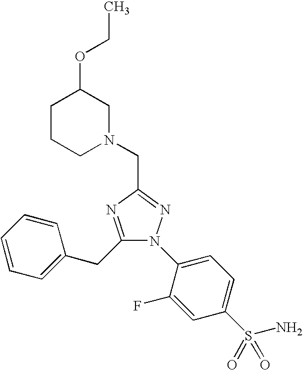 | 474 | 473.2 | L B |
| S-40 | 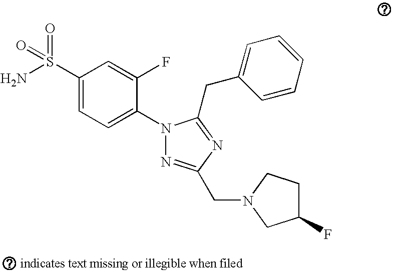 | 434 | 433.1 | L B |
| S-41 | 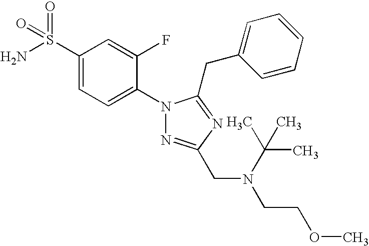 | 476 | 475.2 | L B |
| S-42 | 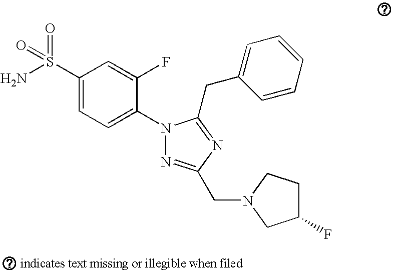 | 434 | 433.1 | L B |
| S-43 | 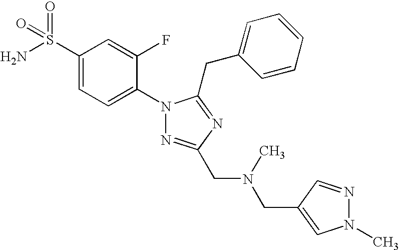 | 470 | 469.2 | L B |
| S-44 | 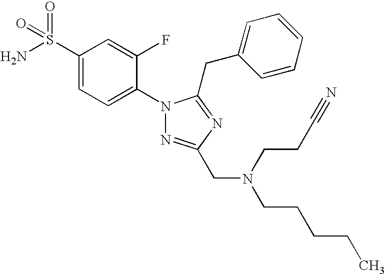 | 485 | 484.2 | L B |
| S-45 | 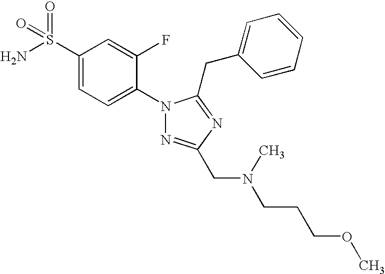 | 448 | 447.2 | L B |
| S-46 | 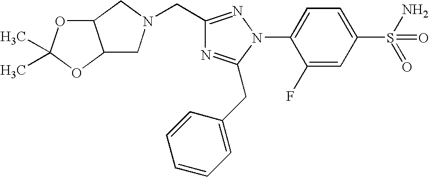 | 488 | 487.2 | L B |
| S-47 | 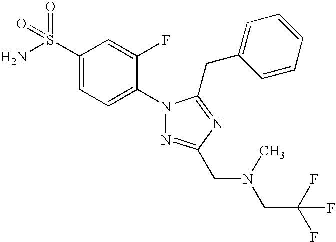 | 458 | 457.1 | L B |
| S-48 | 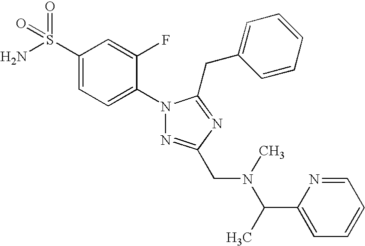 | 481 | 480.2 | L B |
| S-49 | 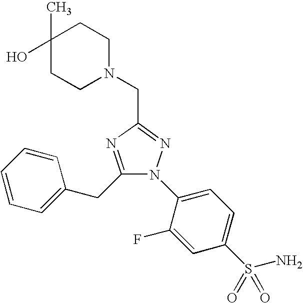 | 460 | 459.2 | L B |
| S-50 | 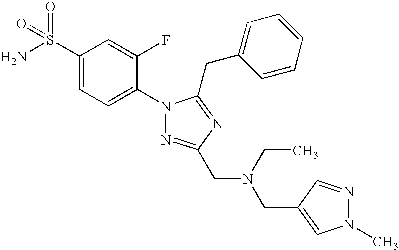 | 484 | 483.2 | L B |
| S-51 | 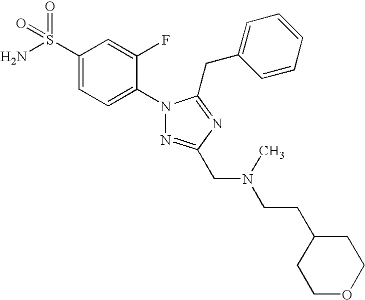 | 488 | 487.2 | L B |
| S-52 | 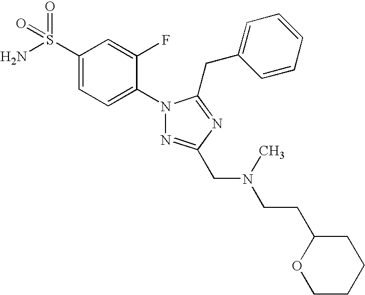 | 488 | 487.2 | L B |
| S-53 | 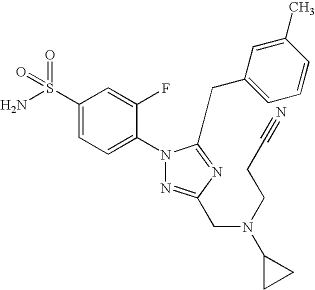 | 469 | 468.2 | L B |
| S-54 | 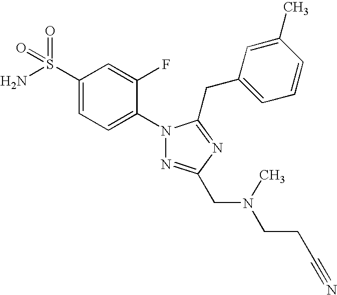 | 443 | 442.2 | L B |
| S-55 | 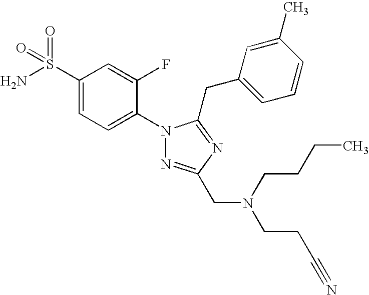 | 485 | 484.2 | L B |
| S-56 | 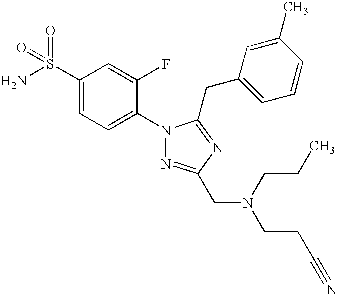 | 471 | 470.2 | L B |
| S-57 | 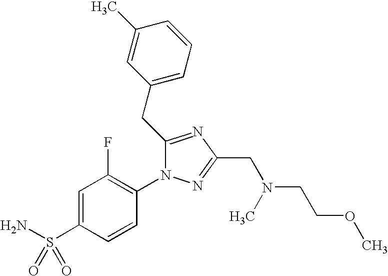 | 448 | 447.2 | L B |
| S-58 | 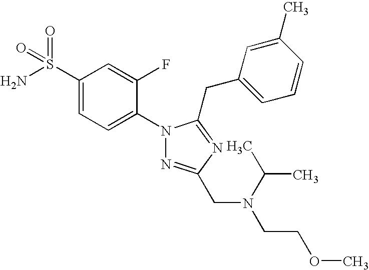 | 476 | 475.2 | L B |
| S-59 | 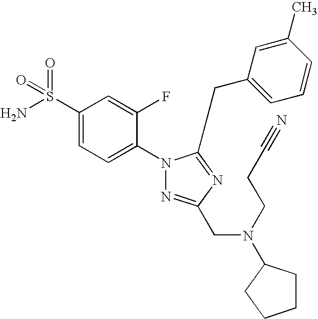 | 497 | 496.2 | L B |
| S-60 | 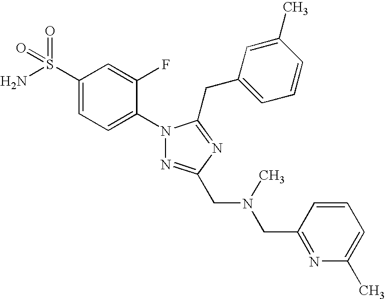 | 495 | 494.2 | L B |
| S-61 | 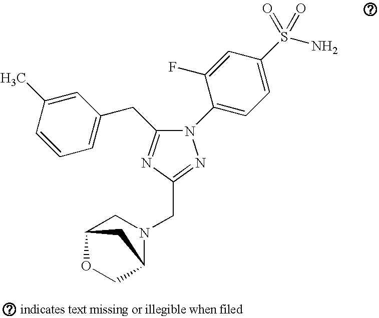 | 458 | 457.2 | L B |
| S-62 | 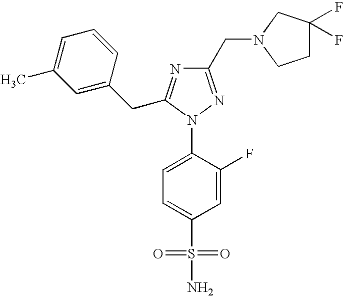 | 466 | 465.1 | L B |
| S-63 | 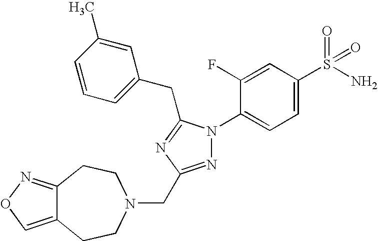 | 497 | 496.2 | L B |
| S-64 | 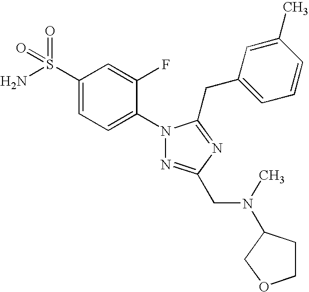 | 460 | 459.2 | L B |
| S-65 | 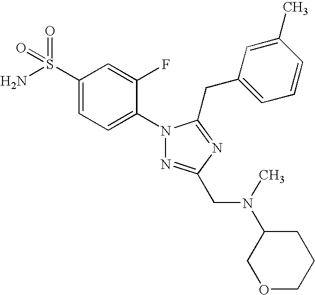 | 474 | 473.2 | L B |
| S-66 | 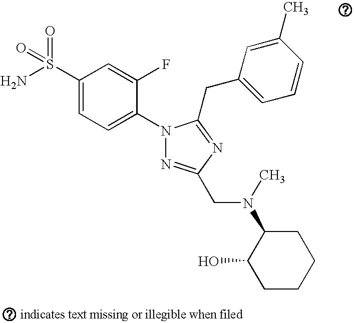 | 488 | 487.2 | L B |
| S-67 | 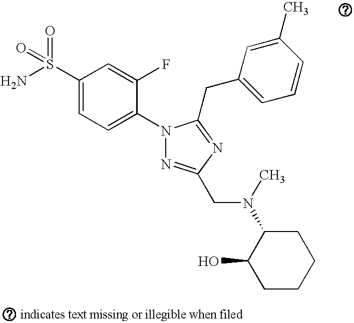 | 488 | 487.2 | L B |
| S-68 | 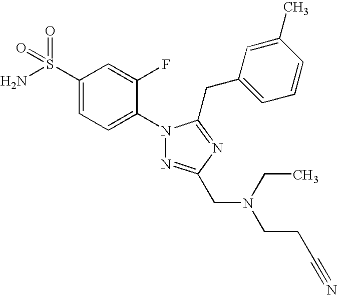 | 457 | 456.2 | L B |
| S-69 | 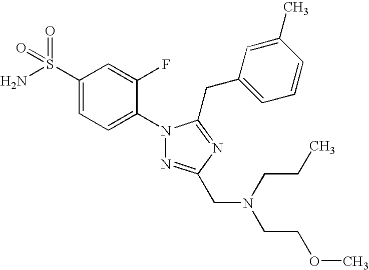 | 476 | 475.2 | L B |
| S-70 | 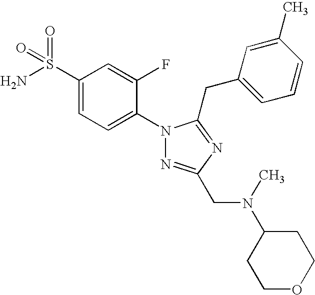 | 474 | 473.2 | L B |
| S-71 | 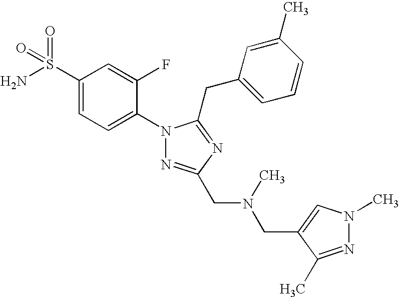 | 498 | 497.2 | L B |
| S-72 | 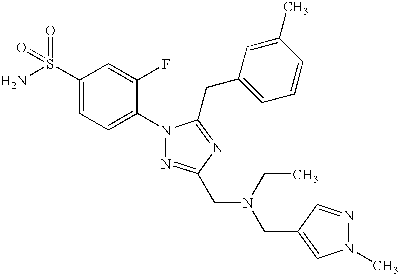 | 498 | 497.2 | L B |
| S-73 | 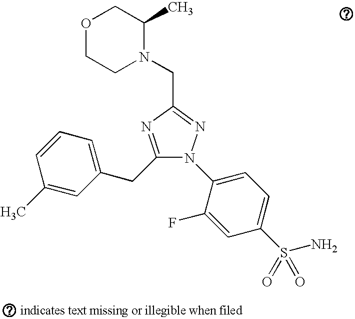 | 460 | 459.2 | L B |
| S-74 | 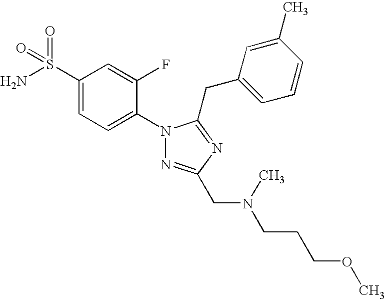 | 462 | 461.2 | L B |
| S-75 | 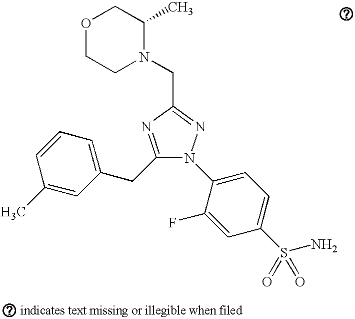 | 460 | 459.2 | L B |
| S-76 | 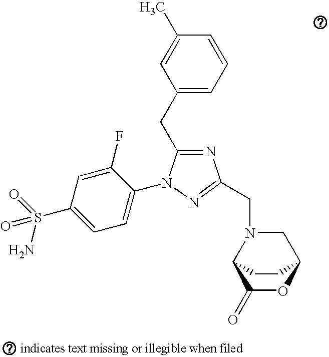 | 486 | 485.2 | L B |
| S-77 | 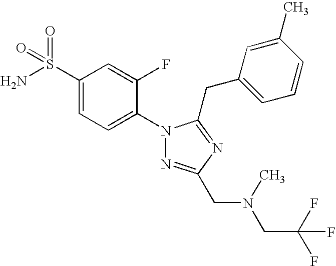 | 472 | 471.1 | L B |
| S-78 | 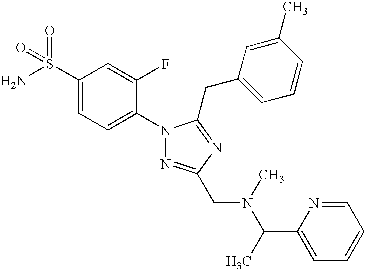 | 495 | 494.2 | L B |
| S-79 | 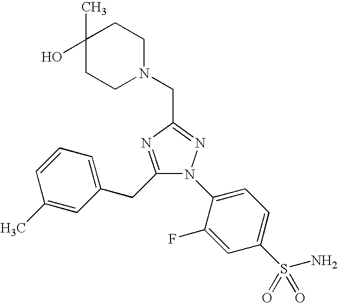 | 474 | 473.2 | L B |
| S-80 | 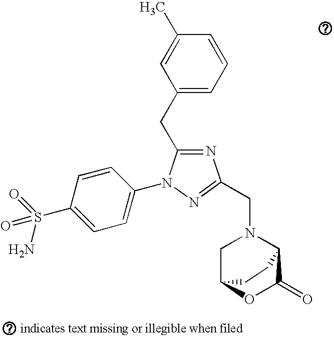 | 486 | 485.2 | L B |
| S-81 | 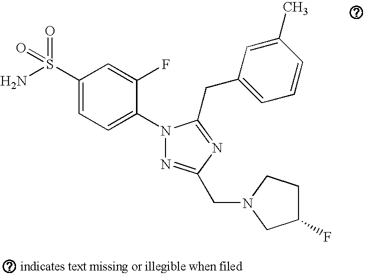 | 448 | 447.2 | L B |
| S-82 | 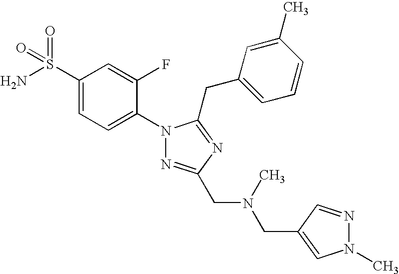 | 484 | 483.2 | L B |
| S-83 | 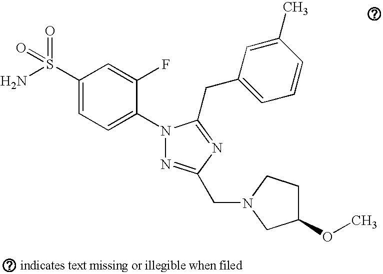 | 460 | 459.2 | L B |
| S-84 | 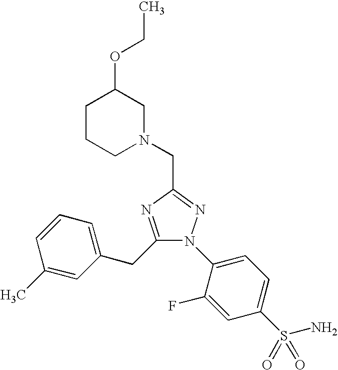 | 488 | 487.2 | L B |
| S-85 | 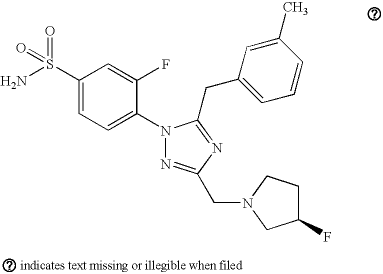 | 448 | 447.2 | L B |
| S-86 | 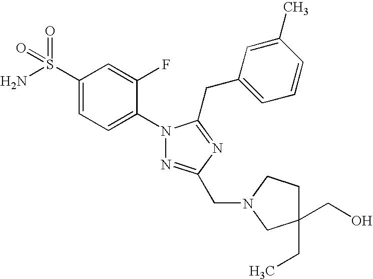 | 488 | 487.2 | L B |
| S-87 | 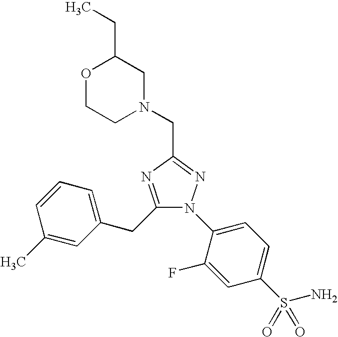 | 474 | 473.2 | L B |
| S-88 | 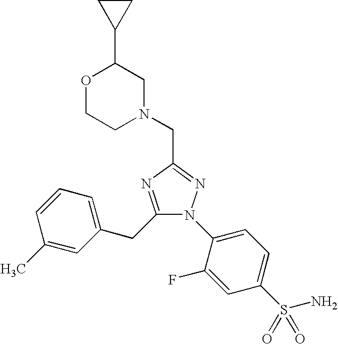 | 486 | 485.2 | L B |
| S-89 | 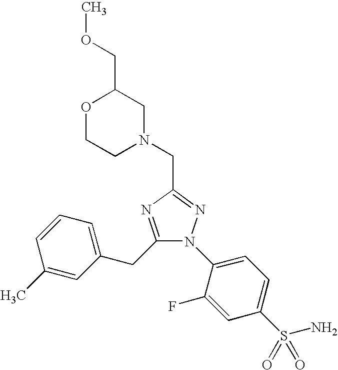 | 490 | 489.2 | L B |
| S-90 | 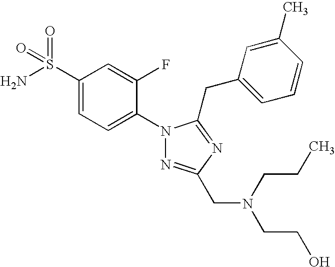 | 462 | 461.2 | L B |
| S-91 | 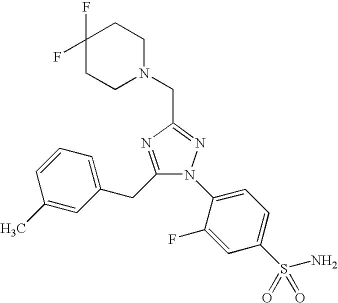 | 480 | 479.2 | L B |
| S-92 | 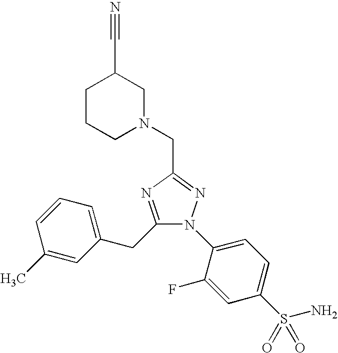 | 469 | 468.2 | L B |
| S-93 | 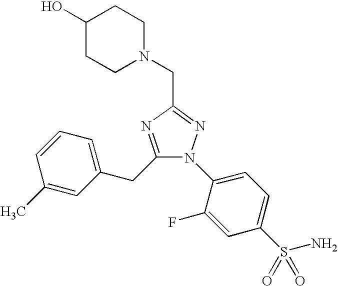 | 460 | 459.2 | L B |
| S-94 | 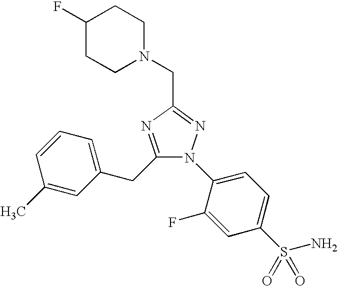 | 462 | 461.2 | L B |
| T-1 | 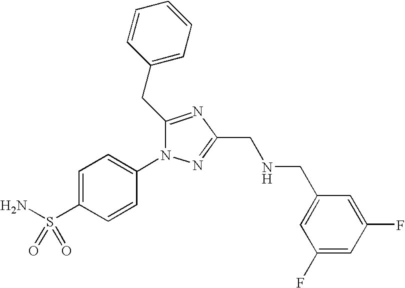 | 470.1 | 469.1 | L C |
| T-2 | 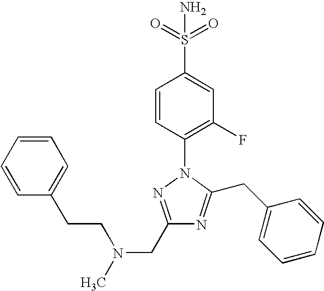 | 480.2 | 479.2 | L C |
| T-3 | 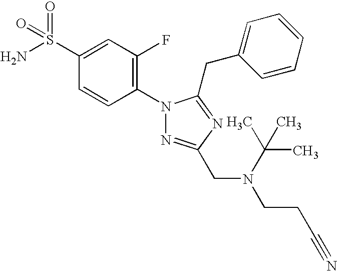 | 471.2 | 470.2 | L C |
| T-4 | 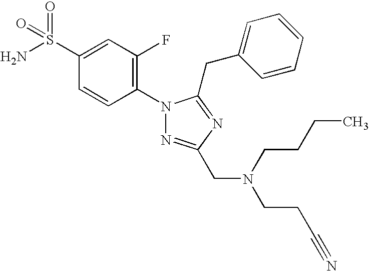 | 471.2 | 470.2 | L C |
| T-5 | 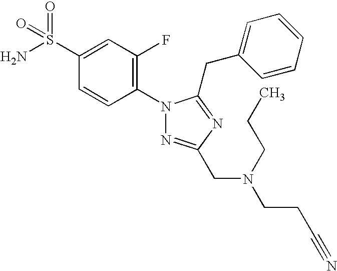 | 457.2 | 456.2 | L C |
| T-6 | 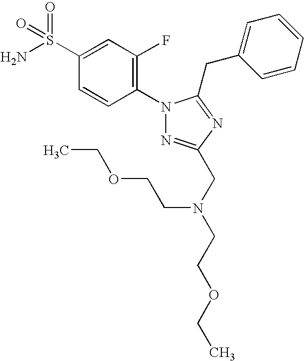 | 506.2 | 505.2 | L C |
| T-7 | 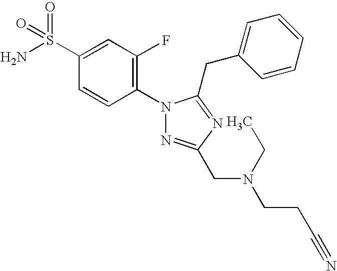 | 443.2 | 442.2 | L C |
| T-8 | 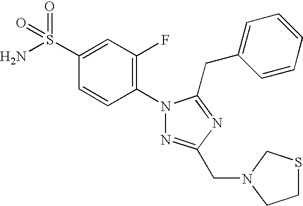 | 434.1 | 433.1 | L C |
| T-9 | 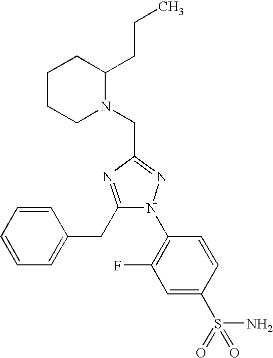 | 472.2 | 471.2 | L C |
| T-10 | 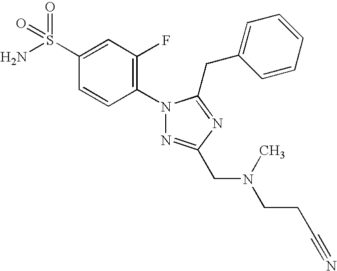 | 429.1 | 428.1 | L C |
| T-11 | 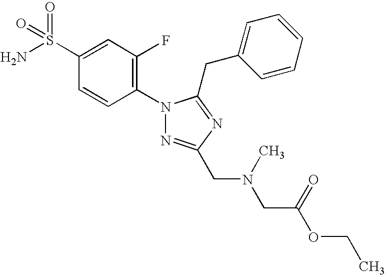 | 462.2 | 461.2 | L C |
| T-12 | 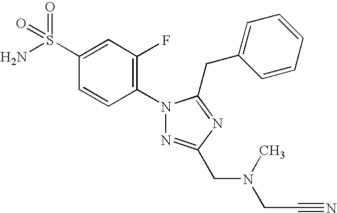 | 415.1 | 414.1 | L C |
| T-13 | 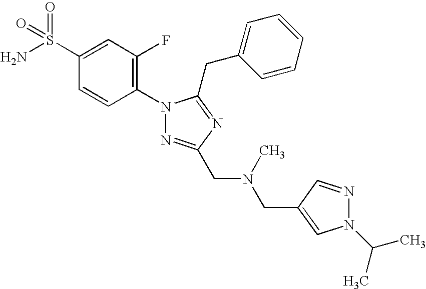 | 498.2 | 497.2 | L C |
| T-14 | 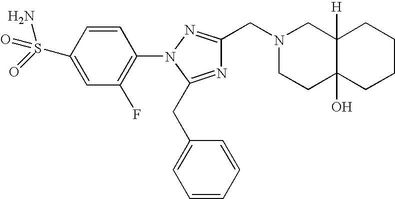 | 500.2 | 499.2 | L C |
| T-15 | 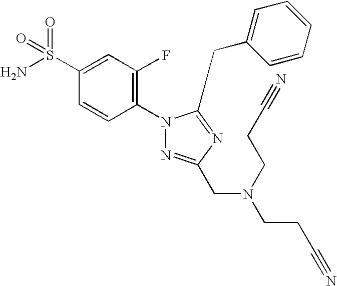 | 468.2 | 467.2 | L C |
| T-16 | 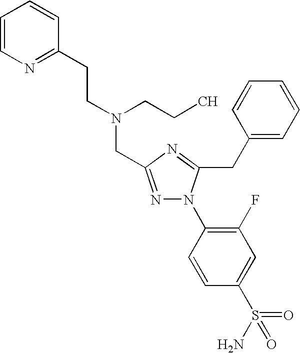 | 509.2 | 508.2 | L C |
| T-17 | 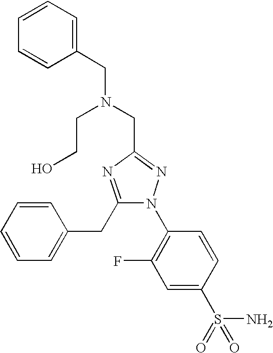 | 496.2 | 495.2 | L C |
| T-18 | 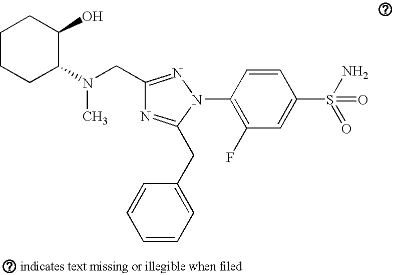 | 474.2 | 473.2 | L C |
| T-19 | 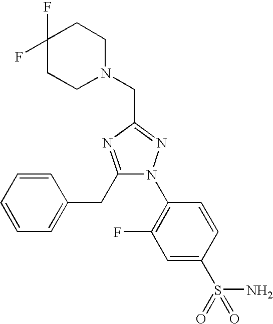 | 466.1 | 465.1 | L C |
| T-20 | 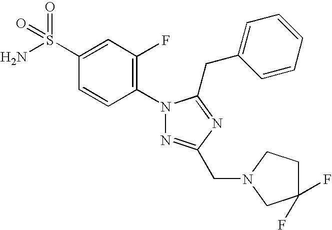 | 452.1 | 451.1 | L C |
| T-21 | 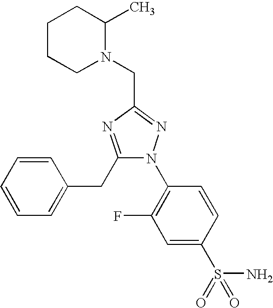 | 444.2 | 443.2 | L C |
| T-22 | 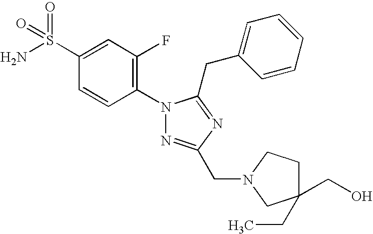 | 474.2 | 473.2 | L C |
| T-23 | 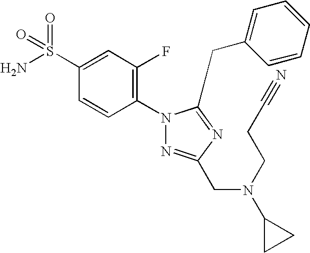 | 455.2 | 454.2 | L C |
| T-24 | 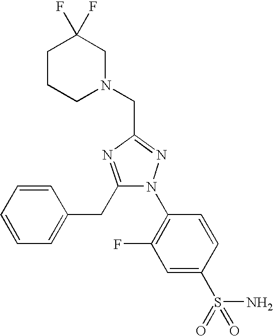 | 466.1 | 465.1 | L C |
| T-25 | 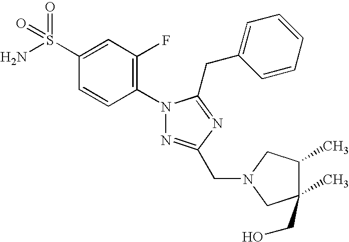 | 474.2 | 473.2 | L C |
| T-26 | 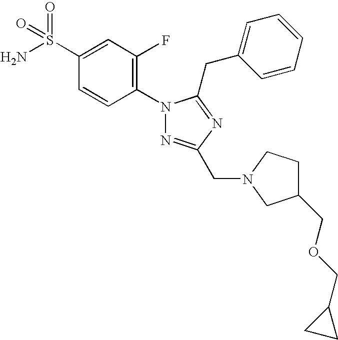 | 500.2 | 499.2 | L C |
| T-27 | 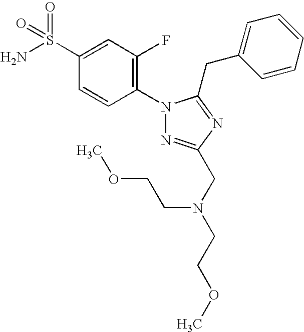 | 478.2 | 477.2 | L C |
| T-26 | 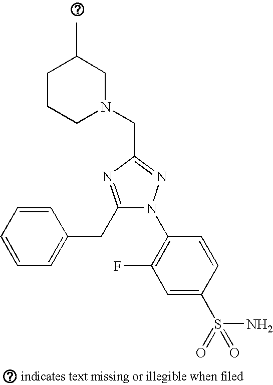 | 455.2 | 454.2 | L C |
| T-29 | 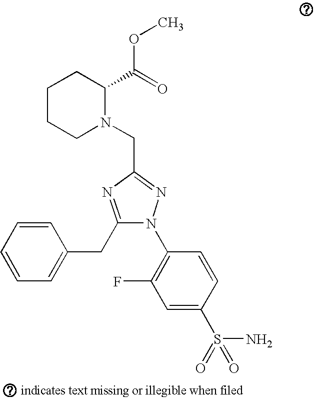 | 488.2 | 487.2 | L C |
| T-30 | 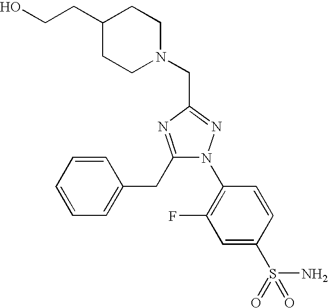 | 474.2 | 473.2 | L C |
| T-31 | 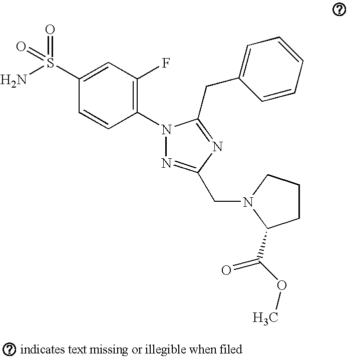 | 474.2 | 473.2 | L C |
| T-32 | 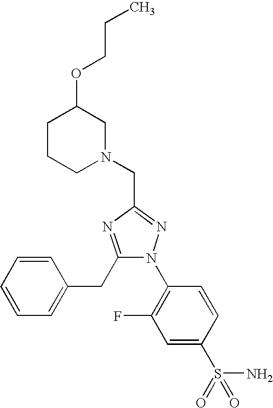 | 488.2 | 487.2 | L C |
| T-33 | 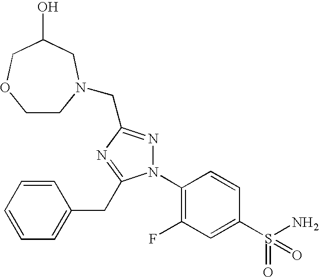 | 462.2 | 461.2 | L C |
| T-34 | 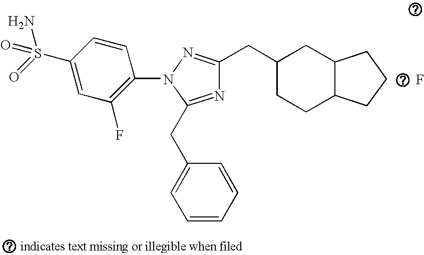 | 489.2 | 488.2 | L C |
| T-35 | 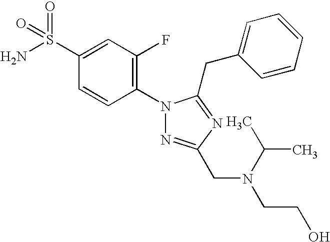 | 448.2 | 447.2 | L C |
| T-36 | 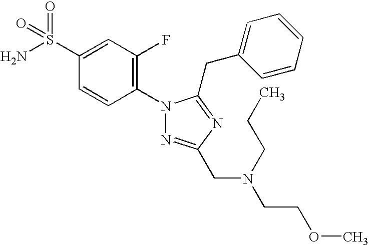 | 462.2 | 461.2 | L C |
| T-37 | 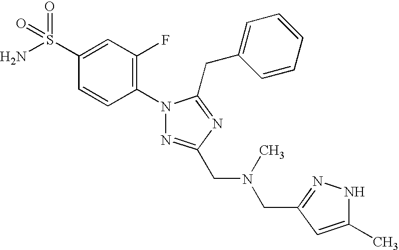 | 470.2 | 469.2 | L C |
| T-38 | 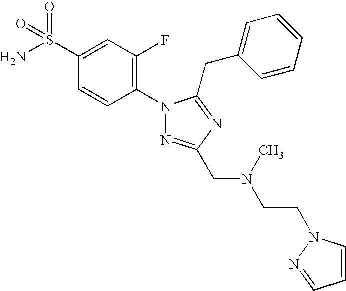 | 470.2 | 469.2 | L C |
| T-39 | 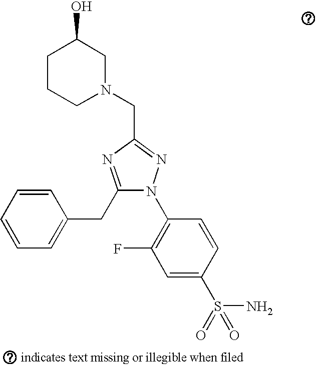 | 446.2 | 445.2 | L C |
| T-40 | 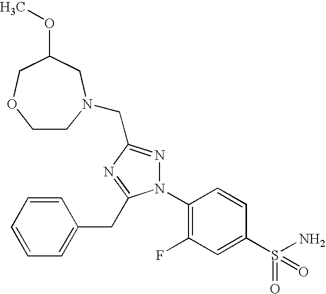 | 476.2 | 475.2 | L C |
| T-41 | 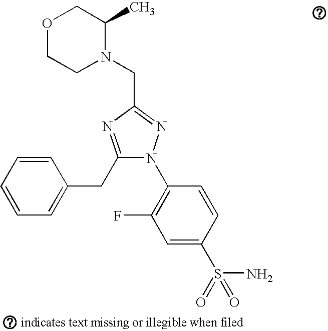 | 446.2 | 445.2 | L C |
| T-42 | 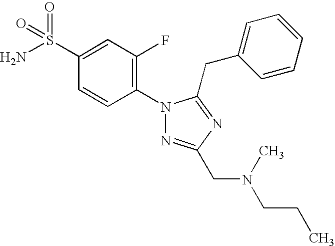 | 418.2 | 417.2 | L C |
| T-43 | 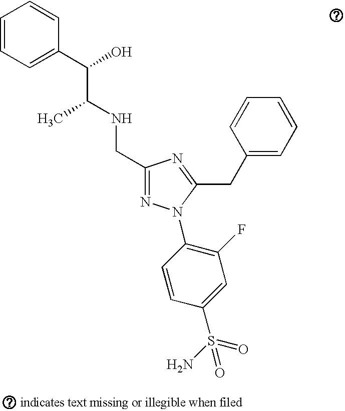 | 496.2 | 495.2 | L C |
| T-44 | 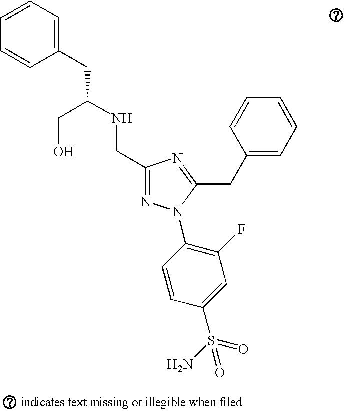 | 496.2 | 495.2 | L C |
| T-45 | 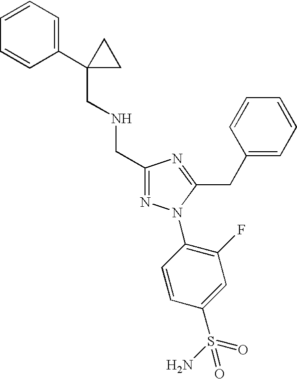 | 492.2 | 491.2 | L C |
| T-46 | 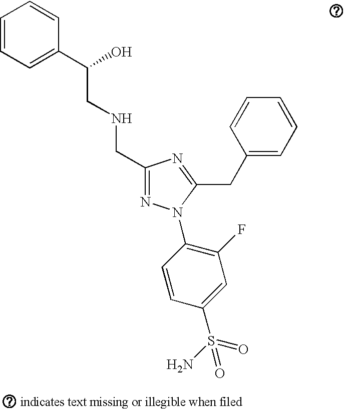 | 482.2 | 481.2 | L C |
| T-47 | 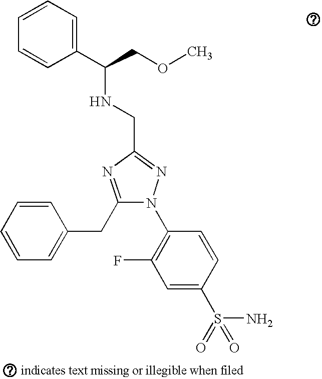 | 496.2 | 495.2 | L C |
| T-48 | 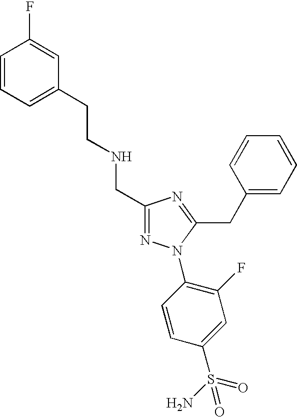 | 484.2 | 483.2 | L C |
| T-49 | 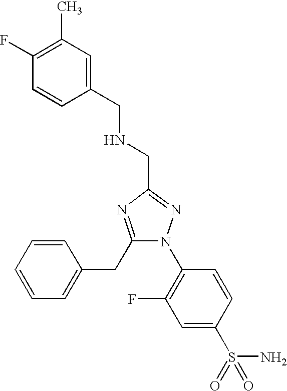 | 484.2 | 483.2 | L C |
| T-50 | 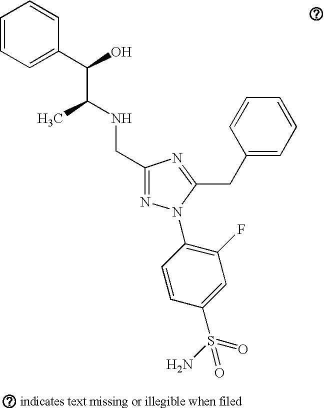 | 496.2 | 495.2 | L C |
| T-51 | 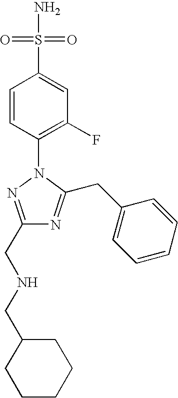 | 458.2 | 457.2 | L C |
| T-52 | 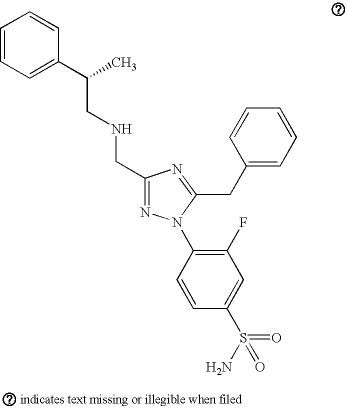 | 480.2 | 479.2 | L C |
| T-53 | 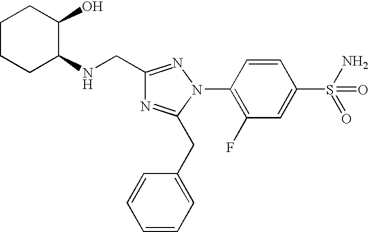 | 460.2 | 459.2 | L C |
| T-54 | 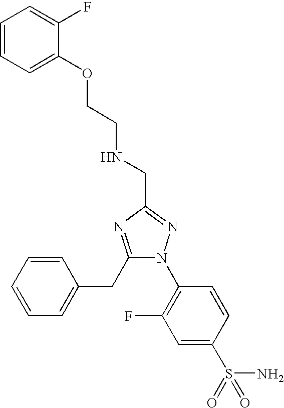 | 500.1 | 499.1 | L C |
| T-55 | 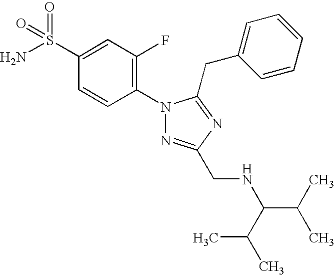 | 460.2 | 459.2 | L C |
| T-56 | 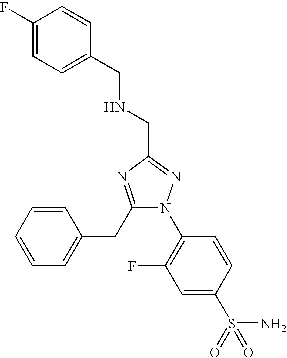 | 470.1 | 469.1 | L C |
| T-57 | 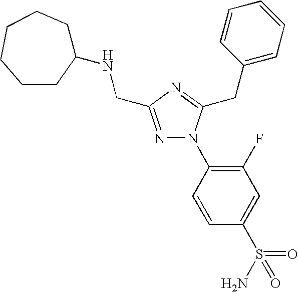 | 458.2 | 457.2 | L C |
| T-58 | 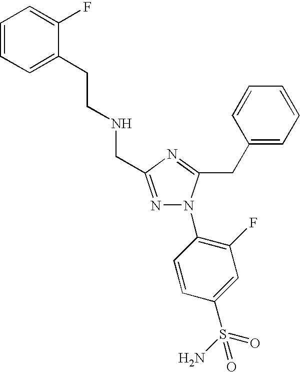 | 484.2 | 483.2 | L C |
| T-59 | 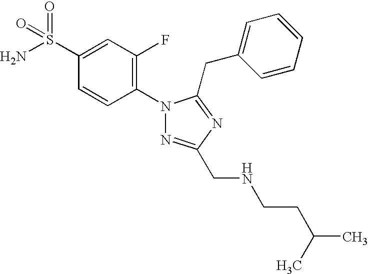 | 432.2 | 431.2 | L C |
| T-60 | 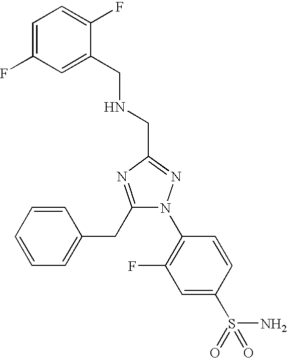 | 488.1 | 487.1 | L C |
| T-61 | 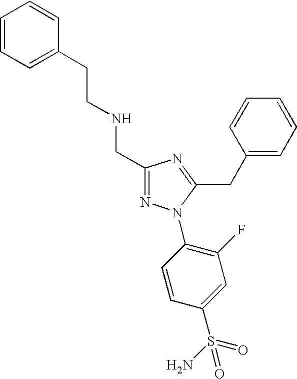 | 466.2 | 465.2 | L C |
| T-62 | 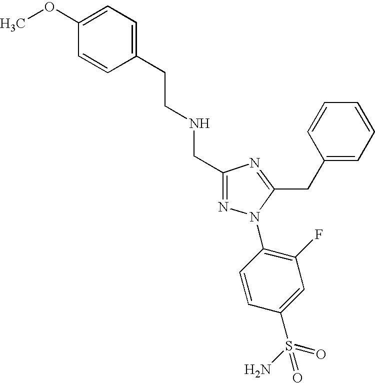 | 496.2 | 495.2 | L C |
| T-63 | 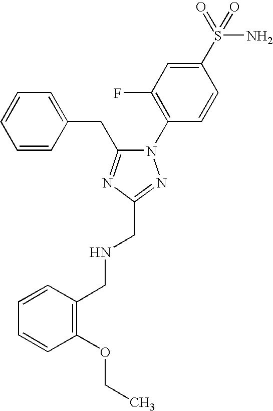 | 496.2 | 495.2 | L C |
| T-64 | 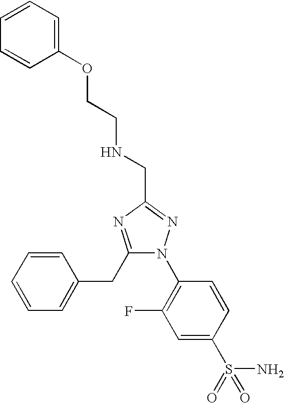 | 482.2 | 481.2 | L C |
| T-65 | 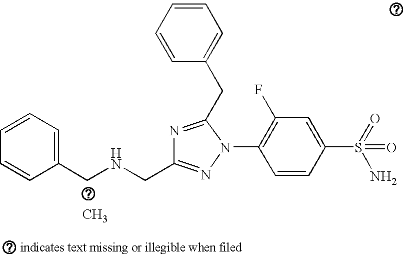 | 466.2 | 465.2 | L C |
| T-66 | 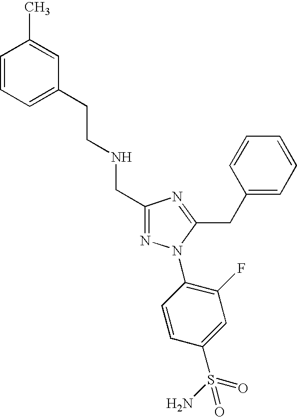 | 480.2 | 479.2 | L C |
| T-67 | 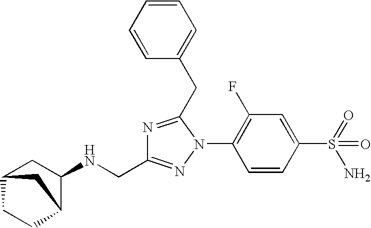 | 456.2 | 455.2 | L C |
| T-68 | 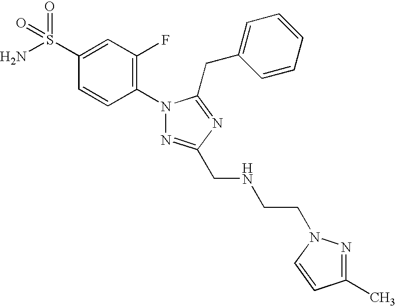 | 470.2 | 469.2 | L C |
| T-69 | 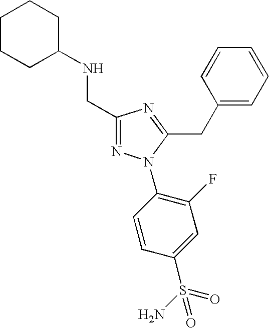 | 444.2 | 443.2 | L C |
| T-70 | 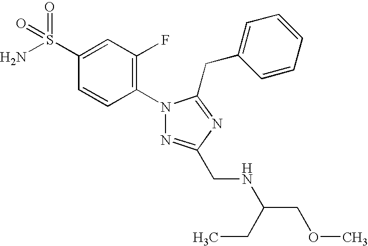 | 448.2 | 447.2 | L C |
| T-71 | 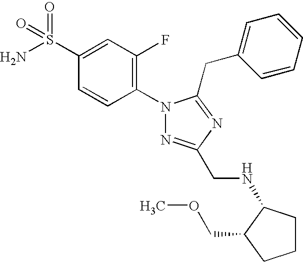 | 474.2 | 473.2 | L C |
| T-72 | 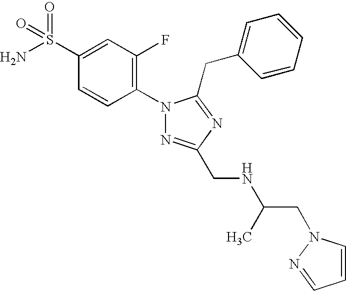 | 470.2 | 469.2 | L C |
| T-73 | 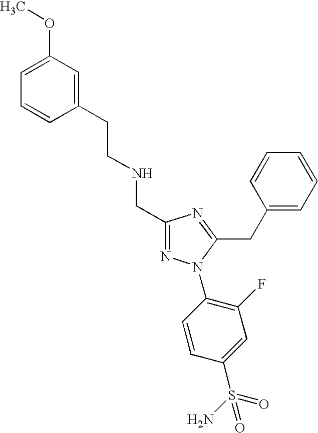 | 496.2 | 495.2 | L C |
| T-74 | 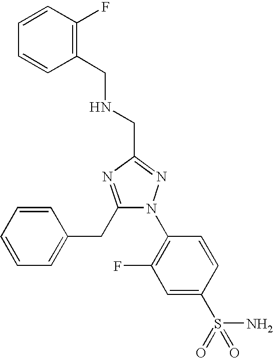 | 470.1 | 469.1 | L C |
| T-75 | 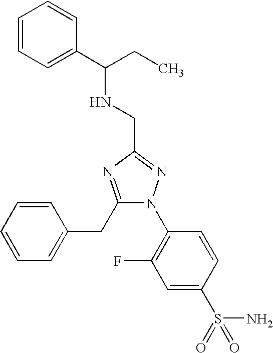 | 480.2 | 479.2 | L C |
| T-76 | 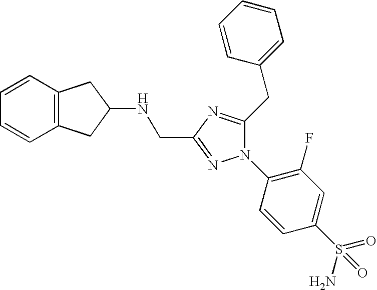 | 478.2 | 477.2 | L C |
| T-77 | 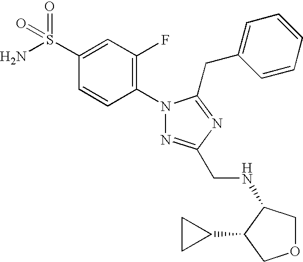 | 472.2 | 471.2 | L C |
| T-78 | 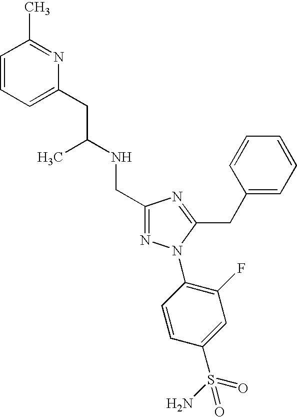 | 495.2 | 494.2 | L C |
| T-79 | 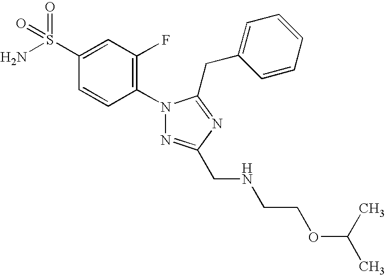 | 448.2 | 447.2 | L C |
| T-80 | 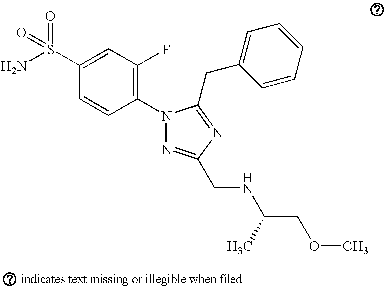 | 434.2 | 433.2 | L C |

| | | | CA- | CA- | | CAII | CA | |
|---|---|---|---|---|---|---|---|---|
| | | CA- | IV: % | IV: | | % Inh | II: | |
| | | IV | Inh @ | Min | CA-II | @ | Min | |
| | | IC50 | Min | dose | Kd | Min | dose | |
| Example # | IUPAC name | (nM) | dose | (nM) | (pM) | dose | (nM) | Purification Method |
| R-1 | 1-[4- | 86 | 68.6 | 200 | 560 | 100 | 100 | LC-G |
| | (aminosulfonyl) | | | | | | | |
| | phenyl]-5- | | | | | | | |
| | benzyl-N- | | | | | | | |
| | methyl-N- | | | | | | | |
| | pentyl-1H-124- | | | | | | | |
| | triazole-3- | | | | | | | |
| | carboxamide | | | | | | | |
| R-2 | 1-[4- | 87 | 57.7 | 200 | 621 | 87 | 10 | LC-G |
| | (aminosulfonyl) | | | | | | | |
| | phenyl]-N5- | | | | | | | |
| | dibenzyl-N-(2- | | | | | | | |
| | hydroxyethyl)- | | | | | | | |
| | 1H-124- | | | | | | | |
| | triazole-3- | | | | | | | |
| | carboxamide | | | | | | | |
| R-3 | 1-[4- | 88 | 66.4 | 200 | 451 | 101 | 100 | LC-G |
| | (aminosulfonyl) | | | | | | | |
| | phenyl]-5- | | | | | | | |
| | benzyl-N-butyl- | | | | | | | |
| | N-methyl-1H- | | | | | | | |
| | 124-triazole-3- | | | | | | | |
| | carboxamide | | | | | | | |
| R-4 | 4-[5-benzyl-3- | 115 | 74.3 | 200 | ND, | 100 | 100 | LC-G |
| | (1245- | | | | IC50 | | | |
| | tetrahydro-3H- | | | | 3.4 nM | | | |
| | 3-benzoazepin- | | | | | | | |
| | 3-ylcarbonyl)- | | | | | | | |
| | 1H-124-triazol- | | | | | | | |
| | 1- | | | | | | | |
| | yl]benzenesul- | | | | | | | |
| | fonamide | | | | | | | |
| R-5 | 1-[4- | 167 | 66.4 | 200 | ND, | 100 | 100 | LC-G |
| | (aminosulfonyl) | | | | IC50 | | | |
| | phenyl]-5- | | | | 2.18 nM | | | |
| | benzyl-N- | | | | | | | |
| | (cyclopropyl- | | | | | | | |
| | methyl)-N-propyl- | | | | | | | |
| | 1H-124- | | | | | | | |
| | triazole-3- | | | | | | | |
| | carboxamide | | | | | | | |
| R-6 | 4-(5-benzyl-3- | 180 | 58.2 | 200 | 325 | 97 | 10 | LC-G |
| | [(44-dimethyl- | | | | | | | |
| | 13-oxazolidin- | | | | | | | |
| | 3-yl)carbonyl]- | | | | | | | |
| | 1H-124-triazol- | | | | | | | |
| | 1- | | | | | | | |
| | yl)benzenesul- | | | | | | | |
| | fonamide | | | | | | | |
| R-7 | 4-[5-benzyl-3- | 182 | 59.1 | 200 | ND, | 101 | 100 | LC-G |
| | (23-dihydro-14- | | | | IC50 | | | |
| | benzoxazepin- | | | | 3.68 nM | | | |
| | 4(5H)- | | | | | | | |
| | ylcarbonyl)-1H- | | | | | | | |
| | 124-triazol-1- | | | | | | | |
| | yl]benzenesul- | | | | | | | |
| | fonamide | | | | | | | |
| R-8 | 4-[3-(2- | 194 | 56.9 | 200 | ND, | 99 | 100 | LC-G |
| | azabicyclo[2.2.1] | | | | 99% at | | | |
| | hept-2- | 100 nM | | | | | | |
| | ylcarbonyl)-5- | | | | | | | |
| | benzyl-1H-124- | | | | | | | |
| | triazol-1- | | | | | | | |
| | yl]benzenesul- | | | | | | | |
| | fonamide | | | | | | | |
| R-9 | 1-[4- | 198 | 58 | 200 | ND, | 99 | 100 | LC-G |
| | (aminosulfonyl) | | | | 99% at | | | |
| | phenyl]-5- | | | | 100 nM | | | |
| | benzyl-N-(2- | | | | | | | |
| | hydroxy-2- | | | | | | | |
| | phenylethyl)-N- | | | | | | | |
| | methyl-1H-124- | | | | | | | |
| | triazole-3- | | | | | | | |
| | carboxamide | | | | | | | |
| R-10 | 1-[4- | 202 | 58 | 200 | ND, | 99 | 100 | LC-G |
| | (aminosulfonyl) | | | | 99% at | | | |
| | phenyl]-5- | | | | 100 nM | | | |
| | cyanoethyl)-N- | | | | | | | |
| | methyl-1H-124- | | | | | | | |
| | triazole-3- | | | | | | | |
| | carboxamide | | | | | | | |
| R-11 | 1-[4- | 202 | 64.7 | 200 | 91 | 99 | 100 | LC-G |
| | (aminosulfonyl) | | | | | | | |
| | phenyl]-5- | | | | | | | |
| | benzyl-N- | | | | | | | |
| | (1456- | | | | | | | |
| | tetrahydrocyclo | | | | | | | |
| | penta[c]pyrazol- | | | | | | | |
| | 3-ylmethyl)- | | | | | | | |
| | 1H-124- | | | | | | | |
| | triazole-3- | | | | | | | |
| | carboxamide | | | | | | | |
| R-12 | 1-[4- | 206 | 62.3 | 200 | ND, | 101 | 100 | LC-G |
| | (aminosulfonyl) | | | | 101% | | | |
| | phenyl]-5- | | | | at | | | |
| | benzyl-N-[(1- | | | | 100 nM | | | |
| | phenylcyclopropyl) | | | | | | | |
| | methyl]-1H- | | | | | | | |
| | 124-triazole-3- | | | | | | | |
| | carboxamide | | | | | | | |
| R-13 | 1-[4- | 208 | 63.6 | 200 | 644 | 100 | 100 | LC-G |
| | (aminosulfonyl) | | | | | | | |
| | phenyl]-5- | | | | | | | |
| | benzyl-N-(2- | | | | | | | |
| | cyanoethyl)-N- | | | | | | | |
| | cyclopropyl- | | | | | | | |
| | 1H-124- | | | | | | | |
| | triazole-3- | | | | | | | |
| | carboxamide | | | | | | | |
| R-14 | 1-(4- | 215 | 59.7 | 200 | ND, | 100 | 100 | LC-G |
| | (aminosulfonyl) | | | | 100% | | | |
| | phenyl]-5- | | | | at | | | |
| | benzyl-N-butyl- | | | | 100 nM | | | |
| | N-ethyl-1H- | | | | | | | |
| | 124-triazole-3- | | | | | | | |
| | carboxamide | | | | | | | |
| R-15 | 4-(5-benzyl-3- | 220 | 60.9 | 200 | ND, | 96 | 100 | LC-G |
| | [(3-phenoxy- | | | | 96% at | | | |
| | azetidin-1- | | | | 100 nM | | | |
| | yl)carbonyl]- | | | | | | | |
| | 1H-124-triazol-1- | | | | | | | |
| | yl)benzenesul- | | | | | | | |
| | fonamide | | | | | | | |
| R-16 | 1-[4- | 220 | 61.3 | 200 | ND, | 100 | 100 | LC-G |
| | (aminosulfonyl) | | | | 100% | | | |
| | phenyl]-5- | | | | at | | | |
| | benzyl-N-(2- | | | | 100 nM | | | |
| | hydroxyethyl)- | | | | | | | |
| | N-pentyl-1H- | | | | | | | |
| | 124-triazole-3- | | | | | | | |
| | carboxamide | | | | | | | |
| R-17 | 1-[4- | 242 | 56.9 | 200 | 67 | 101 | 100 | LC-G |
| | (aminosulfonyl) | | | | | | | |
| | phenyl]-5- | | | | | | | |
| | benzyl-N- | | | | | | | |
| | (cyclohexylmeth- | | | | | | | |
| | yl)-1H-124- | | | | | | | |
| | triazole-3- | | | | | | | |
| | carboxamide | | | | | | | |
| R-18 | 4-(3-[(4-acetyl- | 274 | 56.9 | 200 | ND, | 96 | 100 | LC-G |
| | 14-diazepan-1- | | | | 98% at | | | |
| | yl)carbonyl]-5- | | | | 100 nM | | | |
| | benzyl-1H-124- | | | | | | | |
| | triazol-1- | | | | | | | |
| | yl)benzenesul- | | | | | | | |
| | fonamide | | | | | | | |
| R-19 | 1-[4- | 306 | 50.2 | 200 | ND, | 99 | 100 | LC-G |
| | (aminosulfonyl) | | | | IC50 | | | |
| | phenyl)-5- | | | | 3.55 nM | | | |
| | benzyl-N-ethyl- | | | | | | | |
| | N-(4- | | | | | | | |
| | hydroxybutyl)- | | | | | | | |
| | 1H-124- | | | | | | | |
| | triazole-3- | | | | | | | |
| | carboxamide | | | | | | | |
| R-20 | 1-[4- | 355 | 65.2 | 200 | 805 | 100 | 100 | LC-G |
| | (aminosulfonyl) | | | | | | | |
| | phenyl]-5- | | | | | | | |
| | benzyl-N- | | | | | | | |
| | (cyclopropyl- | | | | | | | |
| | methyl)-N-methyl- | | | | | | | |
| | 1H-124- | | | | | | | |
| | triazole-3- | | | | | | | |
| | carboxamide | | | | | | | |
| R-21 | 1-[4- | 356 | 53.6 | 200 | 90 | 100 | 100 | LC-G |
| | (aminosulfonyl) | | | | | | | |
| | phenyl]-5- | | | | | | | |
| | benzyl-N-[(1S)- | | | | | | | |
| | 2-methoxy-1- | | | | | | | |
| | methylethyl]- | | | | | | | |
| | 1H-124- | | | | | | | |
| | triazole-3- | | | | | | | |
| | carboxamide | | | | | | | |
| R-22 | 1-[4- | 361 | 53.6 | 200 | 91 | 100 | 100 | LC-G |
| | (aminosulfonyl) | | | | | | | |
| | phenyl]-5- | | | | | | | |
| | benzyl-N-(3- | | | | | | | |
| | methoxybenzyl)- | | | | | | | |
| | 1H-124- | | | | | | | |
| | triazole-3- | | | | | | | |
| | carboxamide | | | | | | | |
| R-23 | 4-(5-benzyl-3- | 376 | 40.3 | 200 | 294 | 99 | 100 | LC-G |
| | {[(3S)-3- | | | | | | | |
| | (dimethylamino) | | | | | | | |
| | pyrrolidin-1- | | | | | | | |
| | yl]carbonyl}- | | | | | | | |
| | 1H-124-triazol- | | | | | | | |
| | 1- | | | | | | | |
| | yl)benzenesul- | | | | | | | |
| | fonamide | | | | | | | |
| R-24 | 4-[5-benzyl-3- | 360 | 48.6 | 200 | ND, | 99 | 100 | LC-G |
| | (piperidin-1- | | | | IC50 | | | |
| | ylcarbonyl)-1H- | | | | 3.73 nM | | | |
| | 124-triazol-1- | | | | | | | |
| | yl]benzenesul- | | | | | | | |
| | fonamide | | | | | | | |
| R-25 | 1-[4- | 385 | 69.3 | 200 | ND, | 101 | 100 | LC-G |
| | (aminosulfonyl) | | | | IC50 | | | |
| | phenyl]-5- | | | | 2.51 nM | | | |
| | benzyl-N- | | | | | | | |
| | cyclohexyl-N- | | | | | | | |
| | methyl-1H-124- | | | | | | | |
| | triazole-3- | | | | | | | |
| | carboxamide | | | | | | | |
| R-26 | 4-[3-(6- | 386 | 55.8 | 200 | ND, | 99 | 100 | LC-G |
| | azabicyclo[3.2.1] | | | | IC50 | | | |
| | oct-6- | | | | 2.84 nM | | | |
| | ylcarbonyl)-5- | | | | | | | |
| | benzyl-1H-124- | | | | | | | |
| | triazol-1- | | | | | | | |
| | yl]bezenesul- | | | | | | | |
| | fonamide | | | | | | | |
| R-27 | 4-(5-benzyl-3- | 402 | 53.8 | 200 | ND, | 99 | 100 | LC-G |
| | [(4-formyl-14- | | | | IC50 | | | |
| | diazepan-1- | | | | 4.33 nM | | | |
| | yl)carbonyl]- | | | | | | | |
| | 1H-124-triazol- | | | | | | | |
| | 1- | | | | | | | |
| | yl)benzenesul- | | | | | | | |
| | fonamide | | | | | | | |
| R-28 | 1-[4- | 406 | 66.7 | 200 | ND, | 99 | 100 | LC-G |
| | (aminosulfonyl) | | | | IC50 | | | |
| | phenyl)-5- | | | | 4.16 nM | | | |
| | benzyl-N- | | | | | | | |
| | (dicyclopropyl | | | | | | | |
| | methyl)-N- | | | | | | | |
| | methyl-1H-124- | | | | | | | |
| | triazole-3- | | | | | | | |
| | carboxamide | | | | | | | |
| R-29 | 1-[4- | 412 | 50 | 200 | ND, | 98 | 100 | LC-G |
| | (aminosulfonyl) | | | | IC50 | | | |
| | phenyl]-5- | | | | 5.41 nM | | | |
| | benzyl-N-[(1S)- | | | | | | | |
| | 2-hydroxy-1- | | | | | | | |
| | methylethyl]-N- | | | | | | | |
| | propyl-1H-124- | | | | | | | |
| | triazole-3- | | | | | | | |
| | carboxamide | | | | | | | |
| R-30 | 1-[4- | 415 | 56.4 | 200 | ND, | 100 | 100 | LC-G |
| | (aminosulfonyl) | | | | IC50 | | | |
| | phenyl]-5- | | | | 2.45 nM | | | |
| | benzyl-N- | | | | | | | |
| | cyclopentyl-N- | | | | | | | |
| | methyl-1H-124- | | | | | | | |
| | triazole-3- | | | | | | | |
| | carboxamide | | | | | | | |
| R-31 | 1-[4- | 427 | 61.3 | 200 | ND, | 100 | 100 | LC-G |
| | (aminosulfonyl) | | | | IC50 | | | |
| | phenyl]-5- | | | | 3.03 nM | | | |
| | benzyl-N- | | | | | | | |
| | methyl-N-(3- | | | | | | | |
| | methylbutyl)- | | | | | | | |
| | 1H-124- | | | | | | | |
| | triazole-3- | | | | | | | |
| | carboxamide | | | | | | | |
| R-32 | 4-[5-benzyl-3- | 426 | 42.5 | 200 | ND, | 98 | 100 | LC-G |
| | (morpholin-4- | | | | IC50 | | | |
| | ylcarbonyl)-1H- | | | | 3.56 nM | | | |
| | 124-triazol-1- | | | | | | | |
| | yl]benzenesul- | | | | | | | |
| | fonamide | | | | | | | |
| R-33 | 1-[4- | 429 | 54.7 | 200 | 90 | 101 | 100 | LC-G |
| | (aminosulfonyl) | | | | | | | |
| | phenyl]-5- | | | | | | | |
| | benzyl-N-(23- | | | | | | | |
| | dihydro-1H- | | | | | | | |
| | inden-2-yl)-1H- | | | | | | | |
| | 124-triazole-3- | | | | | | | |
| | carboxamide | | | | | | | |
| R-34 | 1-[4- | 434 | 51.4 | 200 | ND, | 99 | 100 | LC-G |
| | (aminosulfonyl) | | | | IC50 | | | |
| | phenyl]-5- | | | | 2.99 nM | | | |
| | benzyl-N-ethyl- | | | | | | | |
| | N-(2- | | | | | | | |
| | methoxyethyl)- | | | | | | | |
| | 1H-124- | | | | | | | |
| | triazole-3- | | | | | | | |
| | carboxamide | | | | | | | |
| R-35 | 1-[4- | 434 | 48.6 | 200 | 187 | 101 | 100 | LC-G |
| | (aminosulfonyl) | | | | | | | |
| | phenyl]-5- | | | | | | | |
| | benzyl-N-[2- | | | | | | | |
| | (pyridin-2- | . | | | | | | |
| | ylamino)ethyl]- | | | | | | | |
| | 1H-124- | | | | | | | |
| | triazole-3- | | | | | | | |
| | carboxamide | | | | | | | |
| R-36 | 1-[4- | 440 | 67.7 | 200 | ND, | 100 | 100 | LC-G |
| | (aminosulfonyl) | | | | IC50 | | | |
| | phenyl]-5- | | | | 3.12 nM | | | |
| | benzyl-N-butyl- | | | | | | | |
| | N-propyl-1H- | | | | | | | |
| | 124-triazole-3- | | | | | | | |
| | carboxamide | | | | | | | |
| R-37 | 1-[4- | 443 | 67.1 | 200 | ND, | 99 | 100 | LC-G |
| | (aminosulfonyl) | | | | IC50 | | | |
| | phenyl]-5- | | | | 2.89 nM | | | |
| | benzyl-N-(4- | | | | | | | |
| | ethylbenzyl)-N- | | | | | | | |
| | methyl-1H-124- | | | | | | | |
| | triazole-3- | | | | | | | |
| | carboxamide | | | | | | | |
| R-38 | 1-[4- | 458 | 65.8 | 200 | ND, | 100 | 100 | LC-G |
| | (aminosulfonyl) | | | | IC50 | | | |
| | phenyl]-5- | | | | 3.13 nM | | | |
| | benzyl-N- | | | | | | | |
| | methyl-N-(4- | | | | | | | |
| | methylbenzyl)- | | | | | | | |
| | 1H-124- | | | | | | | |
| | triazole-3- | | | | | | | |
| | carboxamide | | | | | | | |
| R-39 | 1-[4- | 477 | 43.6 | 200 | ND, | 97 | 100 | LC-G |
| | (aminosulfonyl) | | | | IC50 | | | |
| | phenyl]-5- | | | | 3.62 nM | | | |
| | benzyl-N-[2- | | | | | | | |
| | (diethylamino) | | | | | | | |
| | ethyl]-N-ethyl- | | | | | | | |
| | 1H-124- | | | | | | | |
| | triazole-3- | | | | | | | |
| | carboxamide | | | | | | | |
| R-40 | 1-[4- | 480 | 60.7 | 200 | ND, | 98 | 100 | LC-G |
| | (aminosulfonyl) | | | | IC50 | | | |
| | phenyl]-5- | | | | 3.34 nM | | | |
| | benzyl-NN- | | | | | | | |
| | diisobutyl-1H- | | | | | | | |
| | 124-triazole-3- | | | | | | | |
| | carboxamide | | | | | | | |
| R-41 | 1[4- | 481 | 46.3 | 200 | ND, | 99 | 100 | LC-G |
| | (aminosulfonyl) | | | | IC50 | | | |
| | phenyl]-5- | | | | 3.89 nM | | | |
| | benzyl-N-(2- | | | | | | | |
| | cyanoethyl)-N- | | | | | | | |
| | ethyl-1H-124- | | | | | | | |
| | triazole-3- | | | | | | | |
| | carboxamide | | | | | | | |
| R-42 | 1-[4- | 488 | 50.5 | 200 | ND, | 98 | 100 | LC-G |
| | (aminosulfonyl) | | | | IC50 | | | |
| | phenyl]-5- | | | | 3.78 nM | | | |
| | benzyl-N- | | | | | | | |
| | methyl-N-(2- | | | | | | | |
| | phenoxyethyl)- | | | | | | | |
| | 1H-124- | | | | | | | |
| | triazole-3- | | | | | | | |
| | carboxamide | | | | | | | |
| R-43 | 1-[4- | 488 | 46.5 | 200 | ND, | 100 | 100 | LC-G |
| | (aminosulfonyl) | | | | IC50 | | | |
| | phenyl]-5- | | | | 4.88 nM | | | |
| | benzyl-N-[2- | | | | | | | |
| | (dimethylamino) | | | | | | | |
| | ethyl]-N-ethyl- | | | | | | | |
| | 1H-124- | | | | | | | |
| | triazole-3- | | | | | | | |
| | carboxamide | | | | | | | |
| R-44 | 1-[4- | 77 | 70.4 | 200 | 577 | 93 | 10 | LC-A |
| | (aminosulfonyl) | | | | | | | |
| | phenyl]-5- | | | | | | | |
| | benzyl-N- | | | | | | | |
| | methyl-N- | | | | | | | |
| | (4567- | | | | | | | |
| | tetrahydro-1H- | | | | | | | |
| | indazol-3- | | | | | | | |
| | ylmethyl)-1H- | | | | | | | |
| | 124-triazole-3- | | | | | | | |
| | carboxamide | | | | | | | |
| R-45 | 1-[4- | 102 | 67.7 | 200 | 90 | 95 | 10 | LC-A |
| | (aminosulfonyl) | | | | | | | |
| | phenyl]-5- | | | | | | | |
| | benzyl-N-(2- | | | | | | | |
| | hydroxy-2- | | | | | | | |
| | phenylethyl)-N- | | | | | | | |
| | isopropyl-1H- | | | | | | | |
| | 124-triazole-3- | | | | | | | |
| | carboxamide | | | | | | | |
| R-46 | 4-{5-benzyl-3- | 108 | 67.7 | 200 | 345 | 96 | 10 | LC-A |
| | [(2-methyl-2- | | | | | | | |
| | phenylmorpholin- | | | | | | | |
| | 4-yl)carbonyl]- | | | | | | | |
| | 1H-124-triazol-1- | | | | | | | |
| | yl}benzenesul- | | | | | | | |
| | fonamide | | | | | | | |
| R-47 | 1-[4- | 118 | 60.4 | 200 | ND, | 90 | 10 | LC-A |
| | (aminosulfonyl) | | | | IC50 | | | |
| | phenyl]-5- | | | | 3.4 nM | | | |
| | benzyl-N- | | | | | | | |
| | [(1S2S)-2- | | | | | | | |
| | hydroxy-1- | | | | | | | |
| | methyl-2- | | | | | | | |
| | phenylethyl]-N- | | | | | | | |
| | methyl-1H-124- | | | | | | | |
| | triazole-3- | | | | | | | |
| | carboxamide | | | | | | | |
| R-48 | 1-[4- | 123 | 62.7 | 200 | 89 | 97 | 10 | LC-A |
| | (aminosulfonyl) | | | | | | | |
| | phenyl]-5- | | | | | | | |
| | benzyl-N- | | | | | | | |
| | methyl-N-(2- | | | | | | | |
| | phenylethyl)- | | | | | | | |
| | 1H-124- | | | | | | | |
| | triazole-3- | | | | | | | |
| | carboxamide | | | | | | | |
| R-49 | 1-[4- | 130 | 70.4 | 200 | 344 | 96 | 10 | LC-A |
| | (aminosulfonyl) | | | | | | | |
| | phenyl]-5- | | | | | | | |
| | benzyl-N-[(6- | | | | | | | |
| | fluoro-1H- | | | | | | | |
| | benzimidazol- | | | | | | | |
| | 2-yl)methyl]-N- | | | | | | | |
| | methyl-1H-124- | | | | | | | |
| | triazole-3- | | | | | | | |
| | carboxamide | | | | | | | |
| R-50 | 1-[4- | 156 | 50.5 | 200 | ND, | 95 | 10 | LC-A |
| | (aminosulfonyl) | | | | IC50 | | | |
| | phenyl]-5- | | | | 2.95 nM | | | |
| | benzyl-N- | | | | | | | |
| | methyl-N-[(5- | | | | | | | |
| | methyl-1H- | | | | | | | |
| | pyrazol-3- | | | | | | | |
| | yl)methyl]-1H- | | | | | | | |
| | 124-triazole-3- | | | | | | | |
| | carboxamide | | | | | | | |
| R-51 | 1-[4- | 170 | 56.3 | 200 | 313 | 97 | 10 | LC-A |
| | (aminosulfonyl) | | | | | | | |
| | phenyl]-5- | | | | | | | |
| | benzyl-N- | | | | | | | |
| | methyl-N-[(5- | | | | | | | |
| | propyl-1H- | | | | | | | |
| | pyrazol-3- | | | | | | | |
| | yl)methyl]-1H- | | | | | | | |
| | 124-triazole-3- | | | | | | | |
| | carboxamide | | | | | | | |
| R-52 | 4-[5-benzyl-3- | 177 | 51.3 | 200 | ND, | 96 | 10 | LC-A |
| | (34- | | | | IC50 | | | |
| | dihydroisoqion- | | | | 2.83 nM | | | |
| | olin-2(1H)- | | | | | | | |
| | ylcarbonyl)-1H- | | | | | | | |
| | 124-triazol-1- | | | | | | | |
| | yl]benzenesul- | | | | | | | |
| | fonamide | | | | | | | |
| R-53 | 4-(5-benzyl-3- | 188 | 61.7 | 200 | ND, | 92 | 10 | LC-A |
| | [(2- | | | | IC50 | | | |
| | benzylpiperidin- | | | | 3.36 nM | | | |
| | 1-yl)carbonyl]- | | | | | | | |
| | 1H-124-triazol- | | | | | | | |
| | 1- | | | | | | | |
| | yl)benzenesul- | | | | | | | |
| | fonamide | | | | | | | |
| R-54 | 1-[4- | 204 | 51.8 | 200 | 131 | 97 | 10 | LC-A |
| | (aminosulfonyl) | | | | | | | |
| | phenyl]-5- | | | | | | | |
| | benzyl-N-(2- | | | | | | | |
| | hydroxy-2- | | | | | | | |
| | phenylpropyl)- | | | | | | | |
| | N-methyl-1H- | | | | | | | |
| | 124-triazole-3- | | | | | | | |
| | carboxamide | | | | | | | |
| R-55 | 4-(5-benzyl-3- | 216 | 46.4 | 200 | 154 | 97 | 10 | LC-A |
| | [(3-pyridin-2- | | | | | | | |
| | ylpyrrolidin-1- | | | | | | | |
| | yl)carbonyl]- | | | | | | | |
| | 1H-124-triazol- | | | | | | | |
| | 1- | | | | | | | |
| | yl)benzenesul- | | | | | | | |
| | fonamide | | | | | | | |
| R-56 | 1-[4- | 220 | 49.5 | 200 | 270 | 95 | 10 | LC-A |
| | (aminosulfonyl) | | | | | | | |
| | phenyl]-5- | | | | | | | |
| | benzyl-N- | | | | | | | |
| | methyl-N-[(4- | | | | | | | |
| | methyl-13- | | | | | | | |
| | thiazol-2- | | | | | | | |
| | yl)methyl]-1H- | | | | | | | |
| | 124-triazole-3- | | | | | | | |
| | carboxamide | | | | | | | |
| R-57 | 4-[5-benzyl-3- | 237 | 49 | 200 | 228 | 96 | 10 | LC-A |
| | (67- | | | | | | | |
| | dihydroisoxazolo | | | | | | | |
| | [43-c]pyridin- | | | | | | | |
| | 5(4H)- | | | | | | | |
| | ylcarbonyl)-1H- | | | | | | | |
| | 124-triazol-1- | | | | | | | |
| | yl]benzenesul- | | | | | | | |
| | fonamide | | | | | | | |
| R-58 | 1-[4- | 240 | 56.8 | 200 | 103 | 98 | 10 | LC-A |
| | (aminosulfonyl) | | | | | | | |
| | phenyl]-5- | | | | | | | |
| | benzyl-N- | | | | | | | |
| | propyl-N-(2- | | | | | | | |
| | pyridin-2- | | | | | | | |
| | ylethyl)-1H- | | | | | | | |
| | 124-triazole-3- | | | | | | | |
| | carboxamide | | | | | | | |
| R-59 | 4-(5-benzyl-3- | 246 | 49.5 | 200 | 133 | 97 | 10 | LC-A |
| | {[(3R4R)-3- | | | | | | | |
| | (hydroxymethyl)- | | | | | | | |
| | 4-pyridin-2- | | | | | | | |
| | ylpyrrolidin-1- | | | | | | | |
| | yl]carbonyl}- | | | | | | | |
| | 1H-124-triazol | | | | | | | |
| | 1- | | | | | | | |
| | yl)benzenesul- | | | | | | | |
| | fonamide | | | | | | | |
| R-60 | 1-[4- | 270 | 44.6 | 200 | ND, | 92 | 10 | LC-A |
| | (aminosulfonyl) | | | | 92% at | | | |
| | phenyl]-5- | | | | 10 nM | | | |
| | benzyl-N- | | | | | | | |
| | methyl-N-[(2- | | | | | | | |
| | methyl-13- | | | | | | | |
| | thiazol-4- | | | | | | | |
| | yl)methyl]-1H- | | | | | | | |
| | 124-triazole-3- | | | | | | | |
| | carboxamide | | | | | | | |
| R-61 | 4-[5-benzyl-3- | 281 | 50.4 | 200 | ND, | 94 | 10 | LC-A |
| | (4578- | | | | 94% at | | | |
| | tetrahydro-6H- | | | | 10 nM | | | |
| | isoxazolo[34- | | | | | | | |
| | d]azepin-6- | | | | | | | |
| | ylcarbonyl)-1H- | | | | | | | |
| | 124-triazol-1- | | | | | | | |
| | yl]benzenesul- | | | | | | | |
| | fonamide | | | | | | | |
| R-62 | 1-[4- | 312 | 53.2 | 200 | 352 | 96 | 10 | LC-A |
| | (aminosulfonyl) | | | | | | | |
| | phenyl]-5- | | | | | | | |
| | benzyl-N- | | | | | | | |
| | methyl-N-[2- | | | | | | | |
| | (1H-pyrazol-4- | | | | | | | |
| | yl)ethyl]-1H- | | | | | | | |
| | 124-triazole-3- | | | | | | | |
| | carboxamide | | | | | | | |
| R-63 | 4-(5-benzyl-3- | 312 | 47.3 | 200 | 275 | 95 | 10 | LC-A |
| | {[3-(3-methyl- | | | | | | | |
| | 124-oxadiazol- | | | | | | | |
| | 5-yl)pyrrolidin- | | | | | | | |
| | 1-yl]carbonyl}- | | | | | | | |
| | 1H-124-triazol- | | | | | | | |
| | 1- | | | | | | | |
| | yl)benzenesul- | | | | | | | |
| | fonamide | | | | | | | |
| R-64 | 1-[4- | 315 | 42.7 | 200 | ND, | 94 | 10 | LC-A |
| | (aminosulfonyl) | | | | IC50 | | | |
| | phenyl]-5- | | | | 2.95 nM | | | |
| | benzyl-N- | | | | | | | |
| | methyl-N-[(3- | | | | | | | |
| | methylisoxazol- | | | | | | | |
| | 5-yl)methyl]- | | | | | | | |
| | 1H-124- | | | | | | | |
| | triazole-3- | | | | | | | |
| | carboxamide | | | | | | | |
| R-65 | 1-[4- | 327 | 49.1 | 200 | ND, | 90 | 10 | LC-A |
| | (aminosulfonyl) | | | | IC50 | | | |
| | phenyl]-5- | | | | 4.16 nM | | | |
| | benzyl-N- | | | | | | | |
| | methyl-N-[2-(4- | | | | | | | |
| | methyl-13- | | | | | | | |
| | thiazol-5- | | | | | | | |
| | yl)ethyl]-1H- | | | | | | | |
| | 124-triazole-3- | | | | | | | |
| | carboxamide | | | | | | | |
| R-66 | 1-[4- | 340 | 50 | 200 | ND, | 88 | 10 | LC-A |
| | (aminosulfonyl) | | | | IC50 | | | |
| | phenyl]-5- | | | | 3.55 nM | | | |
| | benzyl-N-[2- | | | | | | | |
| | (35-dimethyl- | | | | | | | |
| | 1H-pyrazol-1- | | | | | | | |
| | yl)ethyl]-N- | | | | | | | |
| | methyl-1H-124- | | | | | | | |
| | triazole-3- | | | | | | | |
| | carboxamide | | | | | | | |
| R-67 | 4-(5-benzyl-3- | 349 | 42.7 | 200 | ND, | 97 | 10 | LC-A |
| | {[3-(1H- | | | | IC50 | | | |
| | pyrazol-3- | | | | 3.72 nM | | | |
| | yl)piperidin-1- | | | | | | | |
| | yl]carbonyl}- | | | | | | | |
| | 1H-124-triazol- | | | | | | | |
| | 1-yl)benzenesul- | | | | | | | |
| | fonamide | | | | | | | |
| R-68 | 4-(5-benzyl-3- | 353 | 46.4 | 200 | ND, | 92 | 10 | LC-A |
| | {[2-(pyridin-3- | | | | IC50 | | | |
| | ylmethyl)pyrrol- | | | | 3.75 nM | | | |
| | idin-1- | | | | | | | |
| | yl]carbonyl}- | | | | | | | |
| | 1H-124-triazol- | | | | | | | |
| | 1-yl)benzenesul- | | | | | | | |
| | fonamide | | | | | | | |
| R-69 | 4-{5-benzyl-3- | 369 | 49 | 200 | ND, | 97 | 10 | LC-A |
| | [(3-pyridin-3- | | | | IC50 | | | |
| | ylpyrrolidin-1- | | | | 3.49 nM | | | |
| | yl)carbonyl]- | | | | | | | |
| | 1H-124-triazol-1- | | | | | | | |
| | yl}benzenesul- | | | | | | | |
| | fonamide | | | | | | | |
| R-70 | 1-[4- | 388 | 43.2 | 200 | ND, | 94 | 10 | LC-A |
| | (aminosulfonyl) | | | | IC50 | | | |
| | phenyl]-5- | | | | 3.06 nM | | | |
| | benzyl-N-[(5- | | | | | | | |
| | ethyl-124- | | | | | | | |
| | oxadiazol-3- | | | | | | | |
| | yl)methyl]-N- | | | | | | | |
| | methyl-1H-124- | | | | | | | |
| | triazole-3- | | | | | | | |
| | carboxamide | | | | | | | |
| R-71 | 1-[4- | 399 | 45.5 | 200 | 199 | 97 | 10 | LC-A |
| | (aminosulfonyl) | | | | | | | |
| | phenyl]-5- | | | | | | | |
| | benzyl-N-[(1- | | | | | | | |
| | isopropyl-1H- | | | | | | | |
| | pyrazol-4- | | | | | | | |
| | yl)methyl]-N- | | | | | | | |
| | methyl-1H-124- | | | | | | | |
| | triazole-3- | | | | | | | |
| | carboxamide | | | | | | | |
| R-72 | 1-[4- | 90 | 64.4 | 200 | 720 | 89 | 10 | LC-H |
| | (aminosulfonyl) | | | | | | | |
| | phenyl]-5- | | | | | | | |
| | benzyl-N-(3- | | | | | | | |
| | cyclopentylpro- | | | | | | | |
| | pyl)-N-methyl- | | | | | | | |
| | 1H-124- | | | | | | | |
| | triazole-3- | | | | | | | |
| | carboxamide | | | | | | | |
| R-73 | 4-(5-benzyl-3- | 139 | 55.1 | 200 | ND, | 87 | 10 | LC-H |
| | {[4- | | | | IC50 | | | |
| | (hydroxymethyl)- | | | | 3.5 nM | | | |
| | 4-isopropyl- | | | | | | | |
| | piperidin-1- | | | | | | | |
| | yl]carbonyl}- | | | | | | | |
| | 1H-124-triazol- | | | | | | | |
| | 1-yl)benzenesul- | | | | | | | |
| | fonamide | | | | | | | |
| R-74 | 1-[4- | 174 | 62 | 200 | ND, | 95 | 10 | LC-H |
| | (aminosulfonyl) | | | | IC50 | | | |
| | phenyl]-5- | | | | 3.43 nM | | | |
| | benzyl-N-(2- | | | | | | | |
| | cyanoethyl)-N- | | | | | | | |
| | cyclopentyl-1H- | | | | | | | |
| | 124-triazole-3- | | | | | | | |
| | carboxamide | | | | | | | |
| R-75 | 1-[4- | 188 | 65.2 | 200 | ND, | 93 | 10 | LC-H |
| | (aminosulfonyl) | | | | 93% at | | | |
| | phenyl)-5- | | | | 10 nM | | | |
| | benzyl-N-(2- | | | | | | | |
| | cyanoethyl)-N- | | | | | | | |
| | cyclohexyl-1H- | | | | | | | |
| | 124-triazole-3- | | | | | | | |
| | carboxamide | | | | | | | |
| R-76 | 4-(5-benzyl-3- | 189 | 50.9 | 200 | ND, | 88 | 10 | LC-H |
| | {[2-(2- | | | | 88% at | | | |
| | methoxyethyl) | | | | 10 nM | | | |
| | piperidin-1- | | | | | | | |
| | yl]carbonyl}- | | | | | | | |
| | 1H-124-triazol-1- | | | | | | | |
| | yl)benzenesul- | | | | | | | |
| | fonamide | | | | | | | |
| R-77 | 1-[4- | 199 | 50.5 | 200 | ND, | 93 | 10 | LC-H |
| | (aminosulfonyl) | | | | 93% at | | | |
| | phenyl]-5- | | | | 10 nM | | | |
| | benzyl-N-ethyl- | | | | | | | |
| | N-[2-(1H- | | | | | | | |
| | pyrazol-1- | | | | | | | |
| | yl)ethyl]-1H- | | | | | | | |
| | 124-triazole-3- | | | | | | | |
| | carboxamide | | | | | | | |
| R-78 | 4-[5-benzyl-3- | 210 | 45.1 | 200 | ND, | 95 | 10 | LC-H |
| | (octahydropyra- | | | | 95% at | | | |
| | zino[12- | | | | 10 nM | | | |
| | a]azepin- | | | | | | | |
| | 2(1H)- | | | | | | | |
| | ylcarbonyl]-1H- | | | | | | | |
| | 124-triazol-1- | | | | | | | |
| | yl]benzenesul- | | | | | | | |
| | fonamide | | | | | | | |
| R-79 | 1-[4- | 216 | 56.2 | 200 | ND, | 92 | 10 | LC-H |
| | (aminosulfonyl) | | | | 92% at | | | |
| | phenyl]-5- | | | | 10 nM | | | |
| | benzyl-N-butyl- | | | | | | | |
| | N-(2- | | | | | | | |
| | cyanoethyl)- | | | | | | | |
| | 1H-124- | | | | | | | |
| | triazole-3- | | | | | | | |
| | carboxamide | | | | | | | |
| R-80 | 4-[5-benzyl-3- | 218 | 50.3 | 200 | 480 | 95 | 10 | LC-H |
| | (octahydroquin- | | | | | | | |
| | olin-1(2H)- | | | | | | | |
| | ylcarbonyl)-1H- | | | | | | | |
| | 124-triazol-1- | | | | | | | |
| | yl)benzenesul- | | | | | | | |
| | fonamide | | | | | | | |
| R-81 | 4-{5-benzyl-3- | 218 | 53.4 | 200 | ND, | 84 | 10 | LC-H |
| | [(4aS8aS)- | | | | 84% at | | | |
| | octahydroiso- | | | | 10 nM | | | |
| | quinolin-2(1H)- | | | | | | | |
| | ylcarbonyl]-1H- | | | | | | | |
| | 124-triazol-1- | | | | | | | |
| | yl}benzenesul- | | | | | | | |
| | fonamide | | | | | | | |
| R-82 | 4-[3-(2- | 229 | 49.9 | 200 | 496 | 93 | 10 | LC-H |
| | azaspiro[4.5] | | | | | | | |
| | dec-2-ylcarbonyl)- | | | | | | | |
| | 5-benzyl-1H- | | | | | | | |
| | 124-triazol-1- | | | | | | | |
| | yl)benzenesul- | | | | | | | |
| | fonamide | | | | | | | |
| R-83 | 4-{5-benzyl-3- | 237 | 47.3 | 200 | ND, | 82 | 10 | LC-H |
| | [(2-phenyl- | | | | 82% at | | | |
| | pyrrolidin-1- | | | | 10 nM | | | |
| | yl)carbonyl]- | | | | | | | |
| | 1H-124-triazol-1- | | | | | | | |
| | yl}benzenesul- | | | | | | | |
| | fonamide | | | | | | | |
| R-84 | 4-{5-benzyl-3- | 241 | 55.6 | 200 | ND, | 91 | 10 | LC-H |
| | [(3-isobutoxy- | | | | 91% at | | | |
| | piperidin-1- | | | | 10 nM | | | |
| | yl)carbonyl]- | | | | | | | |
| | 1H-124-triazol-1- | | | | | | | |
| | yl}benzenesul- | | | | | | | |
| | fonamide | | | | | | | |
| R-85 | 1-[4- | 248 | 60.1 | 200 | ND, | 93 | 10 | LC-H |
| | (aminosulfonyl) | | | | 93% at | | | |
| | phenyl]-5- | | | | 10 nM | | | |
| | benzyl-N- | | | | | | | |
| | butyl-N- | | | | | | | |
| | (cyanomethyl)- | | | | | | | |
| | 1H-124- | | | | | | | |
| | triazole-3- | | | | | | | |
| | carboxamide | | | | | | | |
| R-86 | 1-[4- | 248 | 49.9 | 200 | 383 | 94 | 10 | LC-H |
| | (aminosulfonyl) | | | | | | | |
| | phenyl]-5- | | | | | | | |
| | benzyl-N-(2- | | | | | | | |
| | chlorobenzyl)- | | | | | | | |
| | N-methyl-1H- | | | | | | | |
| | 124-triazole-3- | | | | | | | |
| | carboxamide | | | | | | | |
| R-87 | 4-(5-benzyl-3- | 251 | 55.3 | 200 | ND, | 91 | 10 | LC-H |
| | {[(4S7R)-4- | | | | 91% at | | | |
| | methyloctahydro- | | | | 10 nM | | | |
| | 2H-47- | | | | | | | |
| | epoxyisoindol- | | | | | | | |
| | 2-yl]carbonyl)- | | | | | | | |
| | 1H-124-triazol- | | | | | | | |
| | 1- | | | | | | | |
| | yl)benzenesul- | | | | | | | |
| | fonamide | | | | | | | |
| R-88 | 4-(5-benzyl-3- | 266 | 43.2 | 200 | ND, | 88 | 10 | LC-H |
| | {[(2S)-2- | | | | 88% at | | | |
| | (methoxymethyl) | | | | 10 nM | | | |
| | pyrrolidin-1- | | | | | | | |
| | yl]carbonyl}- | | | | | | | |
| | 1H-124-triazol-1- | | | | | | | |
| | yl)benzenesul- | | | | | | | |
| | donamide | | | | | | | |
| R-89 | 1-[4- | 267 | 50.3 | 200 | ND, | 94 | 10 | LC-H |
| | (aminosulfonyl) | | | | 94% at | | | |
| | phenyl]-5- | | | | 10 nM | | | |
| | benzyl-N-ethyl- | | | | | | | |
| | N-(4- | | | | | | | |
| | methylbenzyl)- | | | | | | | |
| | 1H-124- | | | | | | | |
| | triazole-3- | | | | | | | |
| | carboxamide | | | | | | | |
| R-90 | 4-{5-benzyl-3- | 274 | 51 | 200 | 552 | 96 | 10 | LC-H |
| | [(44- | | | | | | | |
| | difluoropiper- | | | | | | | |
| | idin-1- | | | | | | | |
| | yl)carbonyl]- | | | | | | | |
| | 1H-124-triazol- | | | | | | | |
| | 1- | | | | | | | |
| | yl}benzenesul- | | | | | | | |
| | fonamide | | | | | | | |
| R-91 | 4-{5-benzyl-3- | 302 | 50.6 | 200 | ND, | 97 | 10 | LC-H |
| | [(3- | | | | IC50 | | | |
| | benzylazetidin- | | | | 3.19 nM | | | |
| | 1-yl)carbonyl]- | | | | | | | |
| | 1H-124-triazol- | | | | | | | |
| | 1- | | | | | | | |
| | yl}benzenesul- | | | | | | | |
| | fonamide | | | | | | | |
| R-92 | 4-{5-benzyl-3- | 311 | 60.5 | 200 | 225 | 97 | 10 | LC-H |
| | [(3- | | | | | | | |
| | cyclohexylpyrrol- | | | | | | | |
| | idin-1- | | | | | | | |
| | yl)carbonyl]- | | | | | | | |
| | 1H-124-triazol- | | | | | | | |
| | 1- | | | | | | | |
| | yl}benzenesul- | | | | | | | |
| | fonamide | | | | | | | |
| R-93 | 1-[4- | 312 | 44.4 | 200 | 449 | 95 | 10 | LC-H |
| | (aminosulfonyl) | | | | | | | |
| | phenyl]-5- | | | | | | | |
| | benzyl-N-(2- | | | | | | | |
| | fluorobenzyl)- | | | | | | | |
| | N-methyl-1H- | | | | | | | |
| | 124-triazole-3- | | | | | | | |
| | carboxamide | | | | | | | |
| R-94 | 4-{5-benzyl-3- | 321 | 53.9 | 200 | ND, | 87 | 10 | LC-H |
| | [(4- | | | | IC50 | | | |
| | isopropylpiper- | | | | 3.98 nM | | | |
| | idin-1- | | | | | | | |
| | yl)carbonyl]- | | | | | | | |
| | 1H-124-triazol- | | | | | | | |
| | 1- | | | | | | | |
| | yl}benzenesul- | | | | | | | |
| | fonamide | | | | | | | |
| R-95 | 1-[4- | 324 | 49.6 | 200 | ND, | 89 | 10 | LC-H |
| | (aminosulfonyl) | | | | IC50 | | | |
| | phenyl]-5- | | | | 3.35 nM | | | |
| | benzyl-N- | | | | | | | |
| | propyl-N- | | | | | | | |
| | (tetrahydrofur- | | | | | | | |
| | an-2-ylmethyl)- | | | | | | | |
| | 1H-124- | | | | | | | |
| | triazole-3- | | | | | | | |
| | carboxamide | | | | | | | |
| R-96 | 1-[4- | 336 | 59.9 | 200 | ND, | 92 | 10 | LC-H |
| | (aminosulfonyl) | | | | IC50 | | | |
| | phenyl]-5- | | | | 3.34 nM | | | |
| | benzyl-N-(tert- | | | | | | | |
| | butyl)-N-(2- | | | | | | | |
| | cyanoethyl)- | | | | | | | |
| | 1H-124- | | | | | | | |
| | triazole-3- | | | | | | | |
| | carboxamide | | | | | | | |
| R-97 | 4-(5-benzyl-3- | 337 | 48.7 | 200 | 492 | 93 | 10 | LC-H |
| | {[(2R)-2- | | | | | | | |
| | (methoxymethyl) | | | | | | | |
| | pyrrolidin-1- | | | | | | | |
| | yl]carbonyl)- | | | | | | | |
| | 1H-124-triazol- | | | | | | | |
| | 1-yl)benzenesul- | | | | | | | |
| | fonamide | | | | | | | |
| R-98 | 4-{5-benzyl-3- | 338 | 48.1 | 200 | ND, | 85 | 10 | LC-H |
| | [(2- | | | | IC50 | | | |
| | propylpiperidin- | | | | 4.93 nM | | | |
| | 1-yl)carbonyl]- | | | | | | | |
| | 1H-124-triazol- | | | | | | | |
| | 1- | | | | | | | |
| | yl}benzenesul- | | | | | | | |
| | fonamide | | | | | | | |
| R-99 | ethyl N-({1-[4- | 356 | 60.1 | 200 | 475 | 94 | 10 | LC-H |
| | (aminosulfonyl) | | | | | | | |
| | phenyl]-5- | | | | | | | |
| | benzyl-1H-124- | | | | | | | |
| | triazol-3- | | | | | | | |
| | yl}carbonyl)-N- | | | | | | | |
| | methylglycinate | | | | | | | |
| R-100 | 4-(5-benzyl-3- | 360 | 52.4 | 200 | ND, | 90 | 10 | LC-H |
| | {[(1R5S)-3- | | | | IC50 | | | |
| | (hydroxymethyl)- | | | | 3.84 nM | | | |
| | 8- | | | | | | | |
| | azabicyclo[3.2.1] | | | | | | | |
| | oct-8- | | | | | | | |
| | yl]carbonyl}- | | | | | | | |
| | 1H-124-triazol- | | | | | | | |
| | 1- | | | | | | | |
| | yl)benzenesul- | | | | | | | |
| | fonamide | | | | | | | |
| R-101 | 1-[4- | 361 | 44.4 | 200 | ND, | 88 | 10 | LC-H |
| | (aminosulfonyl) | | | | IC50 | | | |
| | phenyl]-5- | | | | 3.48 nM | | | |
| | benzyl-N- | | | | | | | |
| | methyl-N-(222- | | | | | | | |
| | trifluoroethyl)- | | | | | | | |
| | 1H-124- | | | | | | | |
| | triazole-3- | | | | | | | |
| | carboxamide | | | | | | | |
| R-102 | 1-[4- | 362 | 48.3 | 200 | ND, | 92 | 10 | LC-H |
| | (aminosulfonyl) | | | | | | | |
| | phenyl]-N5- | | | | UC50 | | | |
| | dibenzyl-N- | | | | 3 nM | | | |
| | ethyl-1H-124- | | | | | | | |
| | triazole-3- | | | | | | | |
| | carboxamide | | | | | | | |
| R-103 | 1-[4- | 363 | 49.9 | 200 | ND, | 88 | 10 | LC-H |
| | (aminosulfonyl) | | | | IC50 | | | |
| | phenyl]-5- | 3.12 nM | | | | | | |
| | benzyl-N-(3- | | | | | | | |
| | methoxybenzyl)- | | | | | | | |
| | N-methyl-1H- | | | | | | | |
| | 124-triazole-3- | | | | | | | |
| | carboxamide | | | | | | | |
| R-104 | 4-{5-benzyl-3- | 367 | 53.5 | 200 | ND, | 89 | 10 | LC-H |
| | [(2- | | | | IC50 | | | |
| | isopropylpiper- | | | | 3.51 nM | | | |
| | idin-1- | | | | | | | |
| | yl)carbonyl]- | | | | | | | |
| | 1H-124-triazol- | | | | | | | |
| | 1-yl}benzenesul- | | | | | | | |
| | fonamide | | | | | | | |
| R-105 | 1-[4- | 368 | 31.7 | 200 | 498 | 84 | 10 | LC-H |
| | (aminosulfonyl) | | | | | | | |
| | phenyl]-5- | | | | | | | |
| | benzyl-N-[3- | | | | | | | |
| | (dimethylamino) | | | | | | | |
| | propyl]-N- | | | | | | | |
| | methyl-1H-124- | | | | | | | |
| | triazole-3- | | | | | | | |
| | carboxamide | | | | | | | |
| R-106 | 1-[4- | 372 | 45.5 | 200 | ND, | 90 | 10 | LC-H |
| | (aminosulfonyl) | | | | IC50 | | | |
| | phenyl]-5- | | | | 3.28 nM | | | |
| | benzyl-N- | | | | | | | |
| | methyl-N- | | | | | | | |
| | (tetrahydro-2H- | | | | | | | |
| | pyran-2- | | | | | | | |
| | ylmethyl)-1H- | | | | | | | |
| | 124-triazole-3- | | | | | | | |
| | carboxamide | | | | | | | |
| R-107 | 1-[4- | 392 | 50.3 | 200 | ND, | 94 | 10 | LC-H |
| | (aminosulfonyl) | | | | IC50 | | | |
| | phenyl]-5- | | | | 3.87 nM | | | |
| | benzyl-N- | | | | | | | |
| | methyl-N-[2- | | | | | | | |
| | (tetrahydro-2H- | | | | | | | |
| | pyran-2- | | | | | | | |
| | yl)ethyl]-1H- | | | | | | | |
| | 124-triazole-3- | | | | | | | |
| | carboxamide | | | | | | | |
| R-108 | 4-{5-benzyl-3- | 414 | 53.8 | 200 | ND, | 86 | 10 | LC-H |
| | [(4aR8aS)- | | | | IC50 | | | |
| | octahydroisoquin- | | | | 4.54 nM | | | |
| | olin-2(1H)- | | | | | | | |
| | ylcarbonyl]-1H- | | | | | | | |
| | 124-triazol-1- | | | | | | | |
| | yl}benzenesul- | | | | | | | |
| | fonamide | | | | | | | |
| R-109 | 1-[4- | 438 | 52.6 | 200 | ND, | 92 | 10 | LC-H |
| | (aminosulfonyl) | | | | IC50 | | | |
| | phenyl]-N5- | | | | 3.61 nM | | | |
| | dibenzyl-N- | | | | | | | |
| | methyl-1H-124- | | | | | | | |
| | triazole-3- | | | | | | | |
| | carboxamide | | | | | | | |
| R-110 | ethyl 1-({1-[4- | 444 | 51 | 200 | ND, | 94 | 10 | LC-H |
| | (aminosulfonyl) | | | | IC50 | | | |
| | phenyl]-5- | | | | 3.9 nM | | | |
| | benzyl-1H-124- | | | | | | | |
| | triazol-3- | | | | | | | |
| | yl]carbonyl) | | | | | | | |
| | piperidine-2- | | | | | | | |
| | carboxylate | | | | | | | |
| R-111 | 4-(5-benzyl-3- | 459 | 44.4 | 200 | ND, | 94 | 10 | LC-H |
| | {[(3R4R)-34- | | | | IC50 | | | |
| | difluoropyrrolidin- | | | | 3.14 nM | | | |
| | 1- | | | | | | | |
| | yl]carbonyl}- | | | | | | | |
| | 1H-124-triazol- | | | | | | | |
| | 1- | | | | | | | |
| | yl)benzenesul- | | | | | | | |
| | fonamide | | | | | | | |
| R-112 | 4-(5-benzyl-3- | 479 | 43.2 | 200 | ND, | 91 | 10 | LC-H |
| | [(3- | | | | IC50 | | | |
| | propoxypiperidin- | | | | 4.44 nM | | | |
| | 1- | | | | | | | |
| | yl)carbonyl]- | | | | | | | |
| | 1H-124-triazol- | | | | | | | |
| | 1- | | | | | | | |
| | yl}benzenesul- | | | | | | | |
| | fonamide | | | | | | | |
| R-113 | 1-[4- | 173 | 61.4 | 200 | 101 | 97 | 10 | LC-B |
| | (aminosulfonyl) | | | | | | | |
| | phenyl]-5- | | | | | | | |
| | benzyl-N-[2-(4- | | | | | | | |
| | fluorophenyl) | | | | | | | |
| | ethyl]-1H-124- | | | | | | | |
| | triazole-3- | | | | | | | |
| | carboxamide | | | | | | | |
| R-114 | 1-[4- | 294 | 57.5 | 200 | 52 | 98 | 10 | LC-B |
| | (aminosulfonyl) | | | | | | | |
| | phenyl]-5- | | | | | | | |
| | benzyl-N-(2- | | | | | | | |
| | phenylethyl)- | | | | | | | |
| | 1H-124- | | | | | | | |
| | triazole-3- | | | | | | | |
| | carboxamide | | | | | | | |
| R-115 | 1-[4- | 321 | 56.5 | 200 | 262 | 98 | 10 | LC-B |
| | (aminosulfonyl) | | | | | | | |
| | phenyl]-5- | | | | | | | |
| | benzyl-N-[2-(2- | | | | | | | |
| | fluorophenyl) | | | | | | | |
| | ethyl]-1H-124- | | | | | | | |
| | triazole-3- | | | | | | | |
| | carboxamide | | | | | | | |
| R-116 | 1-[4- | 327 | 53.7 | 200 | 108 | 99 | 10 | LC-B |
| | (aminosulfonyl) | | | | | | | |
| | phenyl]-5- | | | | | | | |
| | benzyl-N-[2-(3- | | | | | | | |
| | methoxyphenyl) | | | | | | | |
| | ethyl]-1H-124- | | | | | | | |
| | triazole-3- | | | | | | | |
| | carboxamide | | | | | | | |
| R-117 | 1-[4- | 364 | 46.8 | 200 | 196 | 99 | 10 | LC-B |
| | (aminosulfonyl) | | | | | | | |
| | phenyl]-5- | | | | | | | |
| | benzyl-N-[2-(1- | | | | | | | |
| | methyl-1H- | | | | | | | |
| | pyrazol-4- | | | | | | | |
| | yl)ethyl]-1H- | | | | | | | |
| | 124-triazole-3- | | | | | | | |
| | carboxamide | | | | | | | |
| R-118 | 1-[4- | 381 | 50.5 | 200 | 131 | 97 | 10 | LC-B |
| | (aminosulfonyl) | | | | | | | |
| | phenyl]-5- | | | | | | | |
| | benzyl-N-[2-(2- | | | | | | | |
| | methoxyphenyl) | | | | | | | |
| | ethyl]-1H-124- | | | | | | | |
| | triazole-3- | | | | | | | |
| | carboxamide | | | | | | | |
| R-119 | 1-[4- | 414 | 46.1 | 200 | 164 | 100 | 10 | LC-B |
| | (aminosulfonyl) | | | | | | | |
| | phenyl]-5- | | | | | | | |
| | benzyl-N-[3-(4- | | | | | | | |
| | methylpiperidin- | | | | | | | |
| | 1-yl)propyl]- | | | | | | | |
| | 1H-124- | | | | | | | |
| | triazole-3- | | | | | | | |
| | carboxamide | | | | | | | |
| R-120 | 1-[4- | 447 | 55.2 | 200 | 113 | 96 | 10 | LC-B |
| | (aminosulfonyl) | | | | | | | |
| | phenyl]-5- | | | | | | | |
| | benzyl-N-[(1S)- | | | | | | | |
| | 1-benzyl-2- | | | | | | | |
| | hydroxyethyl]- | | | | | | | |
| | 1H-124- | | | | | | | |
| | triazole-3- | | | | | | | |
| | carboxamide | | | | | | | |
| R-121 | 1-[4- | 456 | 46.1 | 200 | 86 | 100 | 10 | LC-B |
| | (aminosulfonyl) | | | | | | | |
| | phenyl]-5- | | | | | | | |
| | benzyl-N-[1- | | | | | | | |
| | (cyclopropyl- | | | | | | | |
| | methyl)piperidin-4- | | | | | | | |
| | yl]-1H-124- | | | | | | | |
| | triazole-3- | | | | | | | |
| | carboxamide | | | | | | | |
| R-122 | 1-[4- | 484 | 46.9 | 200 | 211 | 97 | 10 | LC-B |
| | (aminosulfonyl) | | | | | | | |
| | phenyl]-5- | | | | | | | |
| | benzyl-N-[2-(3- | | | | | | | |
| | methylpyridin- | | | | | | | |
| | 2-yl)ethyl]-1H- | | | | | | | |
| | 124-triazole-3- | | | | | | | |
| | carboxamide | | | | | | | |
| S-1 | 4-(5-benzyl-3- | 142 | 46.8 | 200 | 625 | 97 | 10 | LC-C |
| | {[cyclopentyl | | | | | | | |
| | (methyl)amino] | | | | | | | |
| | methyl}-1H-124- | | | | | | | |
| | triazol-1- | | | | | | | |
| | yl)benzenesul- | | | | | | | |
| | fonamide | | | | | | | |
| S-2 | 4-(5-benzyl-3- | 143 | 45 | 200 | 128 | 98 | 10 | LC-C |
| | [(4- | | | | | | | |
| | formylpiperazin- | | | | | | | |
| | 1-yl)methyl]- | | | | | | | |
| | 1H-124-triazol- | | | | | | | |
| | 1- | | | | | | | |
| | yl]benzenesul- | | | | | | | |
| | fonamide | | | | | | | |
| S-3 | 4-(5-benzyl-3- | 162 | 43 | 200 | 863 | 94 | 10 | LC-C |
| | {[isopropyl(2- | | | | | | | |
| | methoxyethyl) | | | | | | | |
| | amino]methyl}- | | | | | | | |
| | 1H-124-triazol- | | | | | | | |
| | 1- | | | | | | | |
| | yl)benzenesul- | | | | | | | |
| | fonamide | | | | | | | |
| S-4 | 4-(5-benzyl-3- | 204 | 44.2 | 200 | 673 | 94 | 10 | LC-C |
| | {[methyl(2- | | | | | | | |
| | pyridin-2- | | | | | | | |
| | ylethyl)amino] | | | | | | | |
| | methyl}-1H- | | | | | | | |
| | 124-triazol-1- | | | | | | | |
| | yl)benzenesul- | | | | | | | |
| | fonamide | | | | | | | |
| S-5 | 4-[5-benzyl-3- | 221 | 50.6 | 200 | 723 | 94 | 10 | LC-C |
| | (67- | | | | | | | |
| | dihydroisoxazolo | | | | | | | |
| | [43-c]pyridin- | | | | | | | |
| | 5(4H)- | | | | | | | |
| | ylmethyl)-1H- | | | | | | | |
| | 124-triazol-1- | | | | | | | |
| | yl]benzenesul- | | | | | | | |
| | fonamide | | | | | | | |
| S-6 | 4-(5-benzyl-3- | 339 | 45.9 | 200 | ND, | 89 | 10 | LC-C |
| | {[(cyclopropyl- | | | | IC50 | | | |
| | methyl)(propyl) | | | | 3.79 nM | | | |
| | amino]methyl}- | | | | | | | |
| | 1H-124-triazol- | | | | | | | |
| | 1- | | | | | | | |
| | yl)benzenesul- | | | | | | | |
| | fonamide | | | | | | | |
| S-7 | 4-[5-benzyl-3- | 349 | 43.9 | 200 | 821 | 94 | 10 | LC-C |
| | ({[(1R)-2- | | | | | | | |
| | hydroxy-1- | | | | | | | |
| | methylethyl] | | | | | | | |
| | (propyl)amino} | | | | | | | |
| | methyl)-1H-124- | | | | | | | |
| | triazol-1- | | | | | | | |
| | yl]benzenesul- | | | | | | | |
| | fonamide | | | | | | | |
| S-8 | 4-{5-benzyl-3- | 351 | 44.2 | 200 | 815 | 92 | 10 | LC-C |
| | [(3- | | | | | | | |
| | cyanopiperidin- | | | | | | | |
| | 1-yl)methyl]- | | | | | | | |
| | 1H-124-triazol- | | | | | | | |
| | 1- | | | | | | | |
| | yl}benzenesul- | | | | | | | |
| | fonamide | | | | | | | |
| S-9 | 4-(5-benzyl-3- | 355 | 39.1 | 200 | 379 | 93 | 10 | LC-C |
| | {[butyl(methyl) | | | | | | | |
| | amino]methyl}- | | | | | | | |
| | 1H-124-triazol- | | | | | | | |
| | 1-yl)benzenesul- | | | | | | | |
| | fonamide | | | | | | | |
| S-10 | 4-{5-benzyl-3- | 365 | 43.8 | 200 | 980 | 95 | 10 | LC-C |
| | [(4- | | | | | | | |
| | cyanopiperidin- | | | | | | | |
| | 1-yl)methyl]- | | | | | | | |
| | 1H-124-triazol- | | | | | | | |
| | 1- | | | | | | | |
| | yl}benzenesul- | | | | | | | |
| | fonamide | | | | | | | |
| S-11 | 4-(5-benzyl-3- | 369 | 39.6 | 200 | 244 | 99 | 10 | LC-C |
| | {[(2- | | | | | | | |
| | hydroxyethyl) | | | | | | | |
| | (methyl)amino] | | | | | | | |
| | methyl}-1H- | | | | | | | |
| | 124-triazol-1- | | | | | | | |
| | yl)benzenesul- | | | | | | | |
| | fonamide | | | | | | | |
| S-12 | 4-(5-benzyl-3- | 372 | 42.5 | 200 | ND, | 87 | 10 | LC-C |
| | {[butyl(ethyl) | | | | IC50 | | | |
| | amino]methyl}-1H- | | | | 3.43 nM | | | |
| | 124-triazol-1- | | | | | | | |
| | yl)benzenesul- | | | | | | | |
| | fonamide | | | | | | | |
| S-13 | 4-{5-benzyl-3- | 379 | 44.6 | 200 | ND, | 88 | 10 | LC-C |
| | [(12-dimethyl- | | | | IC50 | | | |
| | 3-oxo-2568- | | | | 4.36 nM | | | |
| | tetrahydroimi- | | | | | | | |
| | dazo[15- | | | | | | | |
| | a]pyrazin- | | | | | | | |
| | 7(3H)- | | | | | | | |
| | yl)methyl]-1H- | | | | | | | |
| | 124-triazol-1- | | | | | | | |
| | yl}benzenesul- | | | | | | | |
| | fonamide | | | | | | | |
| S-14 | 4-(5-benzyl-3- | 385 | 43.8 | 200 | ND, | 97 | 10 | LC-C |
| | {[methyl(pyridin- | | | | IC50 | | | |
| | 2- | | | | 3.29 nM | | | |
| | ylmethyl)amino] | | | | | | | |
| | methyl}-1H- | | | | | | | |
| | 124-triazol-1- | | | | | | | |
| | yl)benzenesul- | | | | | | | |
| | fonamide | | | | | | | |
| S-15 | 4-(5-benzyl-3- | 396 | 43.8 | 200 | 690 | 96 | 10 | LC-C |
| | {[(2- | | | | | | | |
| | hydroxyethyl) | | | | | | | |
| | (propyl)amino] | | | | | | | |
| | methyl}-1H-124- | | | | | | | |
| | triazol-1- | | | | | | | |
| | yl)benzenesul- | | | | | | | |
| | fonamide | | | | | | | |
| S-16 | 4-(5-benzyl-3- | 405 | 40.9 | 200 | ND, | 93 | 10 | LC-C |
| | {[4- | | | | IC50 | | | |
| | (hydroxymethyl)- | | | | 3.59 nM | | | |
| | 4- | | | | | | | |
| | methylpiperidin- | | | | | | | |
| | 1-yl]methyl}- | | | | | | | |
| | 1H-124-triazol- | | | | | | | |
| | 1- | | | | | | | |
| | yl)benzenesul- | | | | | | | |
| | fonamide | | | | | | | |
| S-17 | 4-{5-benzyl-3- | 412 | 42.6 | 200 | ND, | 88 | 10 | LC-C |
| | [(3-methyl-56- | | | | IC50 | | | |
| | dihydroimidazo | | | | 3.23 nM | | | |
| | [15-a]pyrazin- | | | | | | | |
| | 7(8H)- | | | | | | | |
| | yl)methyl]-1H- | | | | | | | |
| | 124-triazol-1- | | | | | | | |
| | yl}benzenesul- | | | | | | | |
| | fonamide | | | | | | | |
| S-18 | 4-[5-benzyl-3- | 445 | 40.9 | 200 | 964 | 91 | 10 | LC-C |
| | ({methyl[2-(1H- | | | | | | | |
| | pyrazol-1- | | | | | | | |
| | yl)ethyl]amino} | | | | | | | |
| | methyl)-1H- | | | | | | | |
| | 124-triazol-1- | | | | | | | |
| | yl]benzenesul- | | | | | | | |
| | fonamide | | | | | | | |
| S-19 | 4-(5-benzyl-3- | 449 | 41.2 | 200 | 196 | 96 | 10 | LC-C |
| | {[bis(2- | | | | | | | |
| | hydroxyethyl) | | | | | | | |
| | amino]methyl}- | | | | | | | |
| | 1H-124-triazol- | | | | | | | |
| | 1- | | | | | | | |
| | yl)benzenesul- | | | | | | | |
| | fonamide | | | | | | | |
| S-20 | 4-(5-benzyl-3- | 450 | 43.8 | 200 | 594 | 97 | 10 | LC-C |
| | {[(3R4R)-34- | | | | | | | |
| | difluoropyrrolidin- | | | | | | | |
| | 1-yl]methyl}- | | | | | | | |
| | 1H-124-triazol- | | | | | | | |
| | 1- | | | | | | | |
| | yl)benzenesul- | | | | | | | |
| | fonamide | | | | | | | |
| S-21 | 4-(5-benzyl-3- | 470 | 41.7 | 200 | 732 | 91 | 10 | LC-C |
| | {[(2S4S)-4- | | | | | | | |
| | fluoro-2- | | | | | | | |
| | (hydroxymethyl) | | | | | | | |
| | pyrrolidin-1- | | | | | | | |
| | yl]methyl}-1H- | | | | | | | |
| | 124-triazol-1- | | | | | | | |
| | yl)benzenesul- | | | | | | | |
| | fonamide | | | | | | | |
| S-22 | 4-(5-benzyl-3- | 153 | 81 | 200 | 75 | 89.5 | 2.5 | LC-E |
| | {[benzyl(methyl) | | | | | | | |
| | amino]methyl}- | | | | | | | |
| | 1H-124- | | | | | | | |
| | triazol-1-yl)-3- | | | | | | | |
| | fluorobenzene- | | | | | | | |
| | sulfonamide | | | | | | | |
| S-23 | 4-{5-benzyl-3- | No | 73.1 | 200 | | 94.1 | 2.5 | LC-E |
| | [(diallylamino) | Data | | | | | | |
| | methyl]-1H- | | | | | | | |
| | 124-triazol-1- | | | | | | | |
| | yl}-3- | | | | | | | |
| | fluorobenzene- | | | | | | | |
| | sulfonamide | | | | | | | |
| S-24 | 4-(5-benzyl-3- | 67 | 84.1 | 200 | 41 | 92.4 | 2.5 | LC-E |
| | {[(2- | | | | | | | |
| | cyanoethyl) | | | | | | | |
| | (cyclohexyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-25 | 4-(5-benzyl-3- | 326 | 70.9 | 200 | 102 | 97.7 | 2.5 | LC-E |
| | {[(2- | | | | | | | |
| | methoxyethyl) | | | | | | | |
| | (methyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-26 | 4-(5-benzyl-3- | 308 | 73.1 | 200 | 144 | 74.2 | 2.5 | LC-E |
| | {[isopropyl(2- | | | | | | | |
| | methoxyethyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| S-27 | 4-(5-benzyl-3- | 155 | 85.4 | 200 | 110 | 91.8 | 2.5 | LC-E |
| | {[(2- | | | | | | | |
| | cyanoethyl) | | | | | | | |
| | (cyclopentyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-28 | 4-[5-benzyl-3- | 335 | 74.9 | 200 | 81 | 72.7 | 2.5 | LC-E |
| | ({methyl[(6- | | | | | | | |
| | methylpyridin- | | | | | | | |
| | 2- | | | | | | | |
| | yl)methyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-29 | 4-[5-benzyl-3- | 593 | 67.8 | 200 | 204 | 68.9 | 2.5 | LC-E |
| | (2-oxa-5- | | | | | | | |
| | azabicyclo[2.2.1] | | | | | | | |
| | hept-5- | | | | | | | |
| | ylmethyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-30 | 4-[5-benzyl-3- | 297 | 85.8 | 200 | 211 | 64.5 | 2.5 | LC-E |
| | (13-dihydro- | | | | | | | |
| | 2H-isoindol-2- | | | | | | | |
| | ylmethyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-31 | 4-(5-benzyl-3- | 277 | 70 | 200 | 128 | 73.3 | 2.5 | LC-E |
| | {[propyl(tetra- | | | | | | | |
| | hydrofuran-2- | | | | | | | |
| | ylmethyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-32 | 4-[5-benzyl-3- | 301 | 55.1 | 200 | 58 | 65.7 | 2.5 | LC-E |
| | (4578- | | | | | | | |
| | tetrahydro-6H- | | | | | | | |
| | isoxazolo[34- | | | | | | | |
| | d]azepin-6- | | | | | | | |
| | ylmethyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-33 | 4-(5-benzyl-3- | 335 | 56.4 | 200 | 164 | 73.1 | 2.5 | LC-E |
| | {[methyl(tetra- | | | | | | | |
| | hydrofuran-3- | | | | | | | |
| | yl)amino]methyl}- | | | | | | | |
| | 1H-124- | | | | | | | |
| | triazol-1-yl)-3- | | | | | | | |
| | fluorobenzene- | | | | | | | |
| | sulfonamide | | | | | | | |
| S-34 | 4-(5-benzyl-3- | 379 | 63.9 | 200 | 85 | 77 | 2.5 | LC-E |
| | {[methyl(tetra- | | | | | | | |
| | hydro-2H-pyran- | | | | | | | |
| | 3- | | | | | | | |
| | yl)amino]methyl}- | | | | | | | |
| | 1H-124- | | | | | | | |
| | triazol-1-yl)-3- | | | | | | | |
| | fluorobenzene- | | | | | | | |
| | sulfonamide | | | | | | | |
| S-35 | 4-[5-methyl-3- | 351 | 66.1 | 200 | 77 | 74.8 | 2.5 | LC-E |
| | ({[(13-dimethyl- | | | | | | | |
| | 1H-pyrazol-4- | | | | | | | |
| | yl)methyl](methyl) | | | | | | | |
| | amino}methyl)- | | | | | | | |
| | 1H-124- | | | | | | | |
| | triazol-1-yl]-3- | | | | | | | |
| | fluorobenzene- | | | | | | | |
| | sulfonamide | | | | | | | |
| S-36 | 4-[5-benzyl-3- | No | 60.8 | 200 | | 63.8 | 2.5 | LC-E |
| | ({methyl[2-(2- | Data | | | | | | |
| | methyl-1H- | | | | | | | |
| | imidazol-1- | | | | | | | |
| | yl)ethyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-37 | 4-{5-benzyl-3- | 360 | 65.6 | 200 | 56 | 87.8 | 2.5 | LC-E |
| | [(3- | | | | | | | |
| | fluoropyrrolidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-38 | 4-(5-benzyl-3- | No | 61.3 | 200 | | 64.7 | 2.5 | LC-E |
| | {[(3R)-3- | Data | | | | | | |
| | methoxypyrrol- | | | | | | | |
| | idin-1- | | | | | | | |
| | yl]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-39 | 4-{5-benzyl-3- | 681 | 55.6 | 200 | 233 | 53.8 | 2.5 | LC-E |
| | [(3- | | | | | | | |
| | ethoxypiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-40 | 4-(5-benzyl-3- | 423 | 68.3 | 200 | 45 | 65.2 | 2.5 | LC-E |
| | {[(3R)-3- | | | | | | | |
| | fluoropyrrolidin- | | | | | | | |
| | 1-yl]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| S-41 | 4-(5-benzyl-3- | 227 | 69.2 | 200 | 105 | 47.8 | 2.5 | LC-E |
| | {[tert-butyl(2- | | | | | | | |
| | methoxyethyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| S-42 | 4-(5-benzyl-3- | 376 | 47.7 | 200 | 32 | 55.2 | 2.5 | LC-E |
| | {[(3S)-3- | | | | | | | |
| | fluoropyrrolidin- | | | | | | | |
| | 1-yl]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| S-43 | 4-[5-benzyl-3- | 414 | 53.4 | 200 | 57 | 51.7 | 2.5 | LC-E |
| | ({methyl[(1- | | | | | | | |
| | methyl-1H- | | | | | | | |
| | pyrazol-4- | | | | | | | |
| | yl)methyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-44 | 4-(5-benzyl-3- | 112 | 73.1 | 200 | 144 | 41.9 | 2.5 | LC-E |
| | {[(2- | | | | | | | |
| | cyanoethyl) | | | | | | | |
| | (pentyl)amino] | | | | | | | |
| | methyl}-1H-124- | | | | | | | |
| | triazol-3-yl)-3- | | | | | | | |
| | fluorobenzene- | | | | | | | |
| | sulfonamide | | | | | | | |
| S-45 | 4-(5-benzyl-3- | No | 55.1 | 200 | | 44.5 | 2.5 | LC-E |
| | {[(3- | Data | | | | | | |
| | methoxypropyl) | | | | | | | |
| | (methyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-46 | 4-(5-benzyl-3- | 394 | 53.8 | 200 | 225 | 51.7 | 2.5 | LC-E |
| | {[(3aS6aS)-22- | | | | | | | |
| | dimethyltetrahy- | | | | | | | |
| | dro-5H- | | | | | | | |
| | [13]dioxolo[45- | | | | | | | |
| | c]pyrrol-5- | | | | | | | |
| | yl]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-47 | 4-(5-benzyl-3- | 497 | 55.6 | 200 | 28 | 58.5 | 2.5 | LC-E |
| | {[methyl(222- | | | | | | | |
| | trifluoroethyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| S-48 | 4-(5-benzyl-3- | 294 | 63.9 | 200 | 71 | 61.2 | 2.5 | LC-E |
| | {[methyl(1- | | | | | | | |
| | pyridin-2- | | | | | | | |
| | ylethyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| S-49 | 4-{5-benzyl-3- | 478 | 56 | 200 | 132 | 63.2 | 2.5 | LC-E |
| | [(4-hydroxy-4- | | | | | | | |
| | methylpiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-50 | 4-[5-benzyl-3- | 401 | 68.3 | 200 | 116 | 60.5 | 2.5 | LC-E |
| | ({ethyl[(1- | | | | | | | |
| | methyl-1H- | | | | | | | |
| | pyrazol-4- | | | | | | | |
| | yl)methyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-51 | 4-[5-benzyl-3- | No | 77.5 | 200 | | 72.7 | 2.5 | LC-E |
| | ({methyl[2- | Data | | | | | | |
| | (tetrahydro-2H- | | | | | | | |
| | pyran-4- | | | | | | | |
| | yl)ethyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| S-52 | 4-[5-benzyl-3- | No | 73.5 | 200 | | 89.6 | 2.5 | LC-E |
| | ({methyl[2- | Data | | | | | | |
| | (tetrahydro-2H- | | | | | | | |
| | pyran-2- | | | | | | | |
| | yl)ethyl]amino} | | | | | | | |
| | methyl)-1H- | | | | | | | |
| | 124-triazol-1- | | | | | | | |
| | yl)-3- | | | | | | | |
| | fluorobenzene- | | | | | | | |
| | sulfonamide | | | | | | | |
| S-53 | 4-[3-[{(2- | 173 | 57.3 | 100 | 154 | 63.3 | 2.5 | LC-F |
| | cyanoethyl) | | | | | | | |
| | (cyclopropyl)amino] | | | | | | | |
| | methyl}-5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-54 | 4-[3-{[(2- | 55 | 63.1 | 100 | 103 | 71.3 | 2.5 | LC-F |
| | cyanoethyl) | | | | | | | |
| | (methyl)amino]methyl}- | | | | | | | |
| | 5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-55 | 4-[3-{[butyl(2- | No | 69.8 | 100 | | 81.2 | 2.5 | LC-F |
| | cyanoethyl)amino] | Data | | | | | | |
| | methyl}-5- | | | | | | | |
| | (3-methylbenzyl)- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-56 | 4-[3-{[(2- | No | 63.6 | 100 | | 65.5 | 2.5 | LC-F |
| | cyanoethyl) | Data | | | | | | |
| | (propyl)amino]methyl}- | | | | | | | |
| | 5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-57 | 3-fluoro-4-[3- | 178 | 57.8 | 100 | 80 | 85.6 | 2.5 | LC-F |
| | {[(2- | | | | | | | |
| | methoxyethyl) | | | | | | | |
| | (methyl)amino]- | | | | | | | |
| | methyl}-5-(3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-58 | 3-fluoro-4-[3- | No | 60 | 100 | | 82.5 | 2.5 | LC-F |
| | {[isopropyl(2- | Data | | | | | | |
| | methoxyedthyl) | | | | | | | |
| | amino]methyl}- | | | | | | | |
| | 5-(3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-59 | 4-[3-{[(2- | 70 | 64.5 | 100 | 88 | 70.5 | 2.5 | LC-F |
| | cyanoethyl) | | | | | | | |
| | (cyclopentyl)amino] | | | | | | | |
| | methyl}-5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-60 | 3-fluoro-4-[5- | No | 60.5 | 100 | | 77.4 | 2.5 | LC-F |
| | (3- | Data | | | | | | |
| | methylbenzyl)- | | | | | | | |
| | 3-({methyl[(6- | | | | | | | |
| | methylpyridin- | | | | | | | |
| | 2- | | | | | | | |
| | yl)methyl]amino} | | | | | | | |
| | methyl)-1H- | | | | | | | |
| | 124-triazol-1- | | | | | | | |
| | yl]benzenesul- | | | | | | | |
| | fonamide | | | | | | | |
| S-61 | 3-fluoro-4-{5- | No | 52 | 100 | | 72.2 | 2.5 | LC-F |
| | (3- | Data | | | | | | |
| | methylbenzyl)- | | | | | | | |
| | 3-(2-oxa-5- | | | | | | | |
| | azabicyclo[2.2.1] | | | | | | | |
| | hept-5- | | | | | | | |
| | ylmethyl)-1H- | | | | | | | |
| | 124-triazol-1- | | | | | | | |
| | yl]benzenesul- | | | | | | | |
| | fonamide | | | | | | | |
| S-62 | 4-{3-[(33- | 125 | 64 | 100 | 48 | 60.6 | 2.5 | LC-F |
| | difluoropyrrolidin- | | | | | | | |
| | 1-yl)methyl]- | | | | | | | |
| | 5-(3- | | | | | | | |
| | methylbenzyl)- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-63 | 3-fluoro-4-[5- | 103 | 58.3 | 100 | 80 | 69.3 | 2.5 | LC-F |
| | (3- | | | | | | | |
| | methylbenzyl)- | | | | | | | |
| | 3-(4578- | | | | | | | |
| | tetrahydro-6H- | | | | | | | |
| | isoxazolo[34- | | | | | | | |
| | d]azepin-6- | | | | | | | |
| | ylmethyl)-1H- | | | | | | | |
| | 124-triazol-1- | | | | | | | |
| | yl]benzene- | | | | | | | |
| | sulfonamide | | | | | | | |
| S-64 | 3-fluoro-4-[5- | 102 | 59.1 | 100 | 59 | 85.5 | 2.5 | LC-F |
| | (3- | | | | | | | |
| | methylbenzyl)- | | | | | | | |
| | 3- | | | | | | | |
| | {[methyl(tetra- | | | | | | | |
| | hydrofuran-3- | | | | | | | |
| | yl)amino]methyl}- | | | | | | | |
| | 1H-124- | | | | | | | |
| | triazol-1- | | | | | | | |
| | yl]benzenesul- | | | | | | | |
| | fonamide | | | | | | | |
| S-65 | 3-fluoro-4-[5- | 89 | 56.9 | 100 | 42 | 77 | 2.5 | LC-F |
| | (3- | | | | | | | |
| | methylbenzyl)- | | | | | | | |
| | 3- | | | | | | | |
| | {[methyl(tetra- | | | | | | | |
| | hydro-2H-pyran- | | | | | | | |
| | 3- | | | | | | | |
| | yl)amino]methyl}- | | | | | | | |
| | 1H-124- | | | | | | | |
| | triazol-1- | | | | | | | |
| | yl]benzenesul- | | | | | | | |
| | fonamide | | | | | | | |
| S-66 | 3-fluoro-4-[3- | 75 | 65.8 | 100 | 15 | 79.7 | 2.5 | LC-F |
| | ({[(1S2S)-2- | | | | | | | |
| | hydroxycyclo- | | | | | | | |
| | hexyl](methyl) | | | | | | | |
| | amino}methyl)-5- | | | | | | | |
| | (3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-67 | 3-fluoro-4-[3- | No | 75.1 | 100 | | 88.9 | 2.5 | LC-F |
| | ({[(1R2R)-2- | Data | | | | | | |
| | hydroxycyclo- | | | | | | | |
| | hexyl](methyl) | | | | | | | |
| | amino}methyl)-5- | | | | | | | |
| | (3-methylbenzyl)- | | | | | | | |
| | 1H-124-triazol- | | | | | | | |
| | 1-yl]benzenesul- | | | | | | | |
| | fonamide | | | | | | | |
| S-68 | 4-[3-{[(2- | 104 | 60.9 | 100 | 117 | 70.3 | 2.5 | LC-F |
| | cyanoethyl) | | | | | | | |
| | (ethyl)amino] | | | | | | | |
| | methyl}-5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-69 | 3-fluoro-4-[3- | 118 | 61.3 | 100 | 138 | 78.3 | 2.5 | LC-F |
| | {[(2- | | | | | | | |
| | methoxyethyl) | | | | | | | |
| | (propyl)amino] | | | | | | | |
| | methyl}-5-(3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-70 | 3-fluoro-4-[5- | 121 | 55.6 | 100 | 89 | 70.7 | 2.5 | LC-F |
| | (3-methylbenzyl)- | | | | | | | |
| | 3-{[methyl(tetra- | | | | | | | |
| | hydro-2H- | | | | | | | |
| | pyran-4-yl) | | | | | | | |
| | amino]methyl}- | | | | | | | |
| | 1H-124- | | | | | | | |
| | triazol-1- | | | | | | | |
| | yl]benzenesul- | | | | | | | |
| | fonamide | | | | | | | |
| S-71 | 4-[3-({[(13- | No | 60.4 | 100 | | 71.9 | 2.5 | LC-F |
| | dimethyl-1H- | Data | | | | | | |
| | pyrazol-4- | | | | | | | |
| | yl)methyl](methyl) | | | | | | | |
| | amino}methyl)- | | | | | | | |
| | 5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-72 | 4-[3-({ethyl[(1- | 95 | 62.7 | 100 | 49 | 78.5 | 2.5 | LC-F |
| | methyl-1H- | | | | | | | |
| | pyrazol-4- | | | | | | | |
| | yl)methyl]amino} | | | | | | | |
| | methyl)-5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-73 | 3-fluoro-4-[5- | 103 | 54.7 | 100 | 92 | 80.2 | 2.5 | LC-F |
| | (3- | | | | | | | |
| | methylbenzyl)- | | | | | | | |
| | 3-{[(3R)-3- | | | | | | | |
| | methylmorpholin- | | | | | | | |
| | 4-yl]methyl}- | | | | | | | |
| | 1H-124-triazol- | | | | | | | |
| | 1- | | | | | | | |
| | yl]benzenesul- | | | | | | | |
| | fonamide | | | | | | | |
| S-74 | 3-fluoro-4-[3- | No | 61.8 | 100 | | 84.7 | 2.5 | LC-F |
| | {[(3- | Data | | | | | | |
| | methoxypropyl) | | | | | | | |
| | (methyl)amino] | | | | | | | |
| | methyl}-5-(3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-75 | 3-fluoro-4-[5- | 184 | 51.1 | 100 | 181 | 56.5 | 2.5 | LC-F |
| | (3- | | | | | | | |
| | methylbenzyl)- | | | | | | | |
| | 3-{[(3S)-3- | | | | | | | |
| | methylmorpholin- | | | | | | | |
| | 4-yl]methyl}- | | | | | | | |
| | 1H-124-triazol- | | | | | | | |
| | 1- | | | | | | | |
| | yl]benzenesul- | | | | | | | |
| | fonamide | | | | | | | |
| S-76 | 3-fluoro-4-{5- | No | 60.9 | 100 | | 63.5 | 2.5 | LC-F |
| | (3-methylbenzyl)- | Data | | | | | | |
| | 3-[(3-oxo-2- | | | | | | | |
| | oxa-5- | | | | | | | |
| | azabicyclo[2.2.2] | | | | | | | |
| | oct-5- | | | | | | | |
| | yl)methyl]-1H- | | | | | | | |
| | 124-triazol-1- | | | | | | | |
| | yl}benzenesul- | | | | | | | |
| | fonamide | | | | | | | |
| S-77 | 3-fluoro-4-[5- | 119 | 56 | 100 | 39 | 74.1 | 2.5 | LC-F |
| | (3-methylbenzyl)- | | | | | | | |
| | 3-{[methyl(222- | | | | | | | |
| | trifluoroethyl) | | | | | | | |
| | amino]methyl}- | | | | | | | |
| | 1H-124-triazol- | | | | | | | |
| | 1-yl]benzenesul- | | | | | | | |
| | fonamide | | | | | | | |
| S-78 | 3-fluoro-4-[5- | 108 | 55.1 | 100 | 22 | 58 | 2.5 | LC-F |
| | (3- | | | | | | | |
| | methylbenzyl)- | | | | | | | |
| | 3-{[methyl(1- | | | | | | | |
| | pyridin-2- | | | | | | | |
| | ylethyl)amino] | | | | | | | |
| | methyl}-1H- | | | | | | | |
| | 124-triazol-1- | | | | | | | |
| | yl]benzenesul- | | | | | | | |
| | fonamide | | | | | | | |
| S-79 | 3-fluoro-4-{3- | 173 | 51.1 | 100 | 150 | 65.1 | 2.5 | LC-F |
| | [(4-hydroxy-4- | | | | | | | |
| | methylpiperidin- | | | | | | | |
| | 1-yl)methyl]-5- | | | | | | | |
| | (3-methylbenzyl)- | | | | | | | |
| | 1H-124-triazol- | | | | | | | |
| | 1-yl}benzenesul- | | | | | | | |
| | fonamide | | | | | | | |
| S-80 | 2-fluoro-4-{5- | No | 67.1 | 100 | | 86.9 | 2.5 | LC-F |
| | (3- | Data | | | | | | |
| | methylbenzyl)- | | | | | | | |
| | 3-[(3-oxo-2- | | | | | | | |
| | oxa-5- | | | | | | | |
| | azabicyclo[2.2.2] | | | | | | | |
| | oct-5- | | | | | | | |
| | yl)methyl]-1H- | | | | | | | |
| | 124-triazol-1- | | | | | | | |
| | yl}benzenesul- | | | | | | | |
| | fonamide | | | | | | | |
| S-81 | 3-fluoro-4-[3- | 133 | 59.1 | 100 | 84 | 68.5 | 2.5 | LC-F |
| | {[(3S)-3- | | | | | | | |
| | fluoropyrrolidin- | | | | | | | |
| | 1-yl]methyl}-5- | | | | | | | |
| | (3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-82 | 3-fluoro-4-[5- | 173 | 58.2 | 100 | 71 | > 82.7 | 2.5 | LC-F |
| | (3- | | | | | | | |
| | methylbenzyl)- | | | | | | | |
| | 3-({methyl[(1- | | | | | | | |
| | methyl-1H- | | | | | | | |
| | pyrazol-4- | | | | | | | |
| | yl)methyl]amino} | | | | | | | |
| | methyl)-1H- | | | | | | | |
| | 124-triazol-1- | | | | | | | |
| | yl)benzenesul- | | | | | | | |
| | fonamide | | | | | | | |
| S-83 | 3-fluoro-4-[3- | No | 51.6 | 100 | | 73.2 | 2.5 | LC-F |
| | {[(3R)-3- | Data | | | | | | |
| | methoxypyrrolidin- | | | | | | | |
| | 1-yl]methyl}-5- | | | | | | | |
| | (3-methylbenzyl)- | | | | | | | |
| | 1H-124-triazol- | | | | | | | |
| | 1-yl)benzenesul- | | | | | | | |
| | fonamide | | | | | | | |
| S-84 | 4-{3-[(3- | 165 | 48 | 100 | 205 | 55.4 | 2.5 | LC-F |
| | ethoxypiperidin- | | | | | | | |
| | 1-yl)methyl]-5- | | | | | | | |
| | (3-methylbenzyl)- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-85 | 3-fluoro-4-[3- | 129 | 59.6 | 100 | 167 | 64.1 | 2.5 | LC-F |
| | {[(3R)-3- | | | | | | | |
| | fluoropyrrolidin- | | | | | | | |
| | 1-yl]methyl}-5- | | | | | | | |
| | (3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-86 | 4-[3-{[3-ethyl-3- | No | 62.7 | 100 | | 63.1 | 2.5 | LC-F |
| | (hydroxymethyl) | Data | | | | | | |
| | pyrrolidin-1- | | | | | | | |
| | yl]methyl}-5-(3- | | | | | | | |
| | methylbenzyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| S-87 | 4-{3-[(2- | 174 | 58.3 | 100 | 170 | 57 | 2.5 | LC-F |
| | ethylmorpholin- | | | | | | | |
| | 4-yl)methyl]-5- | | | | | | | |
| | (3-methylbenzyl)- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-88 | 4-{3-[(2- | 139 | 64.5 | 100 | 190 | 77.5 | 2.5 | LC-F |
| | cyclopropylmor | | | | | | | |
| | pholin-4- | | | | | | | |
| | yl)methyl]-5-(3- | | | | | | | |
| | methylbenzyl)- | | | | | | | |
| | 1H-124-triazol- | | | | | | | |
| | 1-yl}-3- | | | | | | | |
| | fluorobenzene- | | | | | | | |
| | sulfonamide | | | | | | | |
| S-89 | 3-fluoro-4-[3- | 265 | 57.8 | 100 | 162 | 79.6 | 2.5 | LC-F |
| | {[2- | | | | | | | |
| | (methoxymethyl) | | | | | | | |
| | morpholin-4- | | | | | | | |
| | yl]methyl}-5-(3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-90 | 3-fluoro-4-[3- | 170 | 51.3 | 100 | 187 | 51 | 2.5 | SF-A |
| | {[(2- | | | | | | | |
| | hydroxyethyl) | | | | | | | |
| | (propyl)amino] | | | | | | | |
| | methyl}-5-(3- | | | | | | | |
| | methylbenzyl)- | | | | | | | |
| | 1H-124-triazol- | | | | | | | |
| | 1- | | | | | | | |
| | yl]benzenesul- | | | | | | | |
| | fonamide | | | | | | | |
| S-91 | 4-{3-[(44- | 118 | 44.7 | 100 | 103 | 58.6 | 2.5 | SF-A |
| | difluoropiperidin- | | | | | | | |
| | 1-yl)methyl]- | | | | | | | |
| | 5-(3- | | | | | | | |
| | methylbenzyl)- | | | | | | | |
| | 1H-124-triazol- | | | | | | | |
| | 1-yl}-3- | | | | | | | |
| | fluorobenzenesul- | | | | | | | |
| | fonamide | | | | | | | |
| S-92 | 4-{3-[(3- | 133 | 54.4 | 100 | 86 | 56.9 | 2.5 | SF-A |
| | cyanopiperidin- | | | | | | | |
| | 1-yl)methyl]-5- | | | | | | | |
| | (3- | | | | | | | |
| | methylbenzyl)- | | | | | | | |
| | 1H-124-triazol- | | | | | | | |
| | 1-yl}-3- | | | | | | | |
| | fluorobenzenesul- | | | | | | | |
| | fonamide | | | | | | | |
| S-93 | 3-fluoro-4-{3- | 236 | 45.1 | 100 | 145 | 51.7 | 2.5 | SF-A |
| | [(4- | | | | | | | |
| | hydroxypiperidin- | | | | | | | |
| | 1-yl)methyl]- | | | | | | | |
| | 5-(3- | | | | | | | |
| | methylbenzyl)- | | | | | | | |
| | 1H-124-triazol- | | | | | | | |
| | 1- | | | | | | | |
| | yl}benzenesul- | | | | | | | |
| | fonamide | | | | | | | |
| S-94 | 3-fluoro-4-{3- | 239 | 48.2 | 100 | 170 | 48.4 | 2.5 | SF-A |
| | [(4- | | | | | | | |
| | fluoropiperidin- | | | | | | | |
| | 1-yl)methyl]-5- | | | | | | | |
| | (3- | | | | | | | |
| | methylbenzyl)- | | | | | | | |
| | 1H-124-triazol- | | | | | | | |
| | 1- | | | | | | | |
| | yl}benzenesul- | | | | | | | |
| | fonamide | | | | | | | |
| T-1 | 4-(5-benzyl-3- | 199 | 40.4 | 200 | 222 | 44 | 2.5 | LC-D |
| | {[(35- | | | | | | | |
| | difluorobenzyl) | | | | | | | |
| | amino]methyl}- | | | | | | | |
| | 1H-124-triazol- | | | | | | | |
| | 1- | | | | | | | |
| | yl)benzenesul- | | | | | | | |
| | fonamide | | | | | | | |
| T-2 | 4-(5-benzyl-3- | 110 | 71.3 | 200 | 127 | 44.0 | 2.5 | SF-A |
| | {[methyl(2- | | No | | | No | | |
| | phenylethyl) | | Data | | | Data | | |
| | amino]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-3 | 4-(5-benzyl-3- | 139 | 64.2 | 200 | 146 | 56 | 2.5 | SF-A |
| | {[tert-butyl(2- | | | | | | | |
| | cyanoethyl) | | | | | | | |
| | amino]methyl}-1H- | | | | | | | |
| | 124-triazol-1- | | | | | | | |
| | yl)-3- | | | | | | | |
| | fluorobenzenesul- | | | | | | | |
| | fonamide | | | | | | | |
| T-4 | 4-(5-benzyl-3- | 156 | 71 | 200 | 122 | 60 | 2.5 | SF-A |
| | {[butyl(2- | | | | | | | |
| | cyanoethyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-5 | 4-(5-benzyl-3- | 169 | 58.3 | 200 | 144 | 58 | 2.5 | SF-A |
| | {[(2- | | | | | | | |
| | cyanoethyl) | | | | | | | |
| | propyl)amino] | | | | | | | |
| | methyl}-1H-124- | | | | | | | |
| | triazol-1-yl)-3- | | | | | | | |
| | fluorobenzene- | | | | | | | |
| | sulfonamide | | | | | | | |
| T-6 | 4-(5-benzyl-3- | 174 | 52.9 | 200 | 451 | 52 | 2.5 | SF-A |
| | {[bis(2- | | | | | | | |
| | ethoxyethyl) | | | | | | | |
| | amino]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-7 | 4-(5-benzyl-3- | 195 | 61.9 | 200 | 133 | 64.5 | 2.5 | SF-A |
| | {[(2- | | | | | | | |
| | cyanoethyl) | | | | | | | |
| | (ethyl)amino] | | | | | | | |
| | methyl}-1H-124- | | | | | | | |
| | triazol-1-yl)-3- | | | | | | | |
| | fluorobenzene- | | | | | | | |
| | sulfonamide | | | | | | | |
| T-8 | 4-[5-benzyl-3- | 207 | 48.8 | 200 | 19 | 68 | 2.5 | SF-A |
| | (13-thiazolidin- | | | | | | | |
| | 3-ylmethyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-9 | 4-{5-benzyl-3- | 230 | 57.5 | 200 | 502 | 53 | 2.5 | SF-A |
| | [(2- | | | | | | | |
| | propylpiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-10 | 4-(5-benzyl-3- | 231 | 63.5 | 200 | 99 | 75.6 | 2.5 | SF-A |
| | {[(2- | | | | | | | |
| | cyanoethyl) | | | | | | | |
| | (methyl)amino]methyl}- | | | | | | | |
| | 1H-124- | | | | | | | |
| | triazol-1-yl)-3- | | | | | | | |
| | fluorobenzene- | | | | | | | |
| | sulfonamide | | | | | | | |
| T-11 | ethyl N-({1-[4- | 236 | 48.4 | 200 | 136 | 58 | 2.5 | SF-A |
| | (aminosulfonyl)- | | | | | | | |
| | 2- | | | | | | | |
| | fluorophenyl]- | | | | | | | |
| | 5-benzyl-1H- | | | | | | | |
| | 124-triazol-3- | | | | | | | |
| | yl}methyl)-N- | | | | | | | |
| | methylglycinate | | | | | | | |
| T-12 | 4-(5-benzyl-3- | 238 | 53.5 | 200 | 89 | 61 | 2.5 | SF-A |
| | {[(cyanomethyl) | | No | | | | | |
| | (methyl)amino] | | Data | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-13 | 4-[5-benzyl-3- | 238 | 52.8 | 200 | 106 | 56 | 2.5 | SF-A |
| | ({[(1-isopropyl- | | | | | | | |
| | 1H-pyrazol-4- | | | | | | | |
| | yl)methyl](methyl) | | | | | | | |
| | amino}methyl)- | | | | | | | |
| | 1H-124- | | | | | | | |
| | triazol-1-yl]-3- | | | | | | | |
| | fluorobenzene- | | | | | | | |
| | sulfonamide | | | | | | | |
| T-14 | 4-(5-benzyl-3- | 239 | 57.2 | 200 | 316 | 43 | 2.5 | SF-A |
| | {[(4aS8aS)-4a- | | | | | | | |
| | hydroxyocta- | | | | | | | |
| | hydroisoquinolin- | | | | | | | |
| | 2(1H)- | | | | | | | |
| | yl]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-15 | 4-(5-benzyl-3- | 241 | 53.1 | 200 | 237 | 51 | 2.5 | SF-A |
| | {[bis(2- | | | | | | | |
| | cyanoethyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-16 | 4-(5-benzyl-3- | 249 | 54.3 | 200 | 236 | 47 | 2.5 | SF-A |
| | {[propyl(2- | | | | | | | |
| | pyridin-2- | | | | | | | |
| | ylethyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-17 | 4-(5-benzyl-3- | 250 | 56.6 | 200 | 58 | 43 | 2.5 | SF-A |
| | {[benzyl(2- | | No | | | | | |
| | hydroxyethyl) | | Data | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-18 | 4-[5-benzyl-3- | 263 | 41.6 | 100 | 38 | 52.8 | 2.5 | SF-A |
| | ({[(1R2R)-2- | | | | | | | |
| | hydroxycyclo- | | | | | | | |
| | hexyl](methyl)amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-19 | 4-{5-benzyl-3- | 274 | 27 | 100 | 216 | 58.9 | 2.5 | SF-A |
| | [(44- | | | | | | | |
| | difluoropiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-20 | 4-{5-benzyl-3- | 291 | 63.1 | 200 | 156 | 68.3 | 2.5 | SF-A |
| | [(33- | | | | | | | |
| | difluoropyrrolidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-21 | 4-{5-benzyl-3- | 302 | 45.5 | 200 | 235 | 69 | 2.5 | SF-A |
| | [(2- | | | | | | | |
| | methylpiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-22 | 4-(5-benzyl-3- | 307 | 52.8 | 200 | 110 | 48 | 2.5 | SF-A |
| | {[3-ethyl-3- | | | | | | | |
| | (hydroxymethyl) | | | | | | | |
| | pyrrolidin-1- | | | | | | | |
| | yl]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-23 | 4-(5-benzyl-3- | 310 | 52.6 | 200 | 190 | 51 | 2.5 | SF-A |
| | {[(2- | | | | | | | |
| | cyanoethyl) | | | | | | | |
| | (cyclopropyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-24 | 4-{5-benzyl-3- | 313 | 31.4 | 100 | 88 | 55.7 | 2.5 | SF-A |
| | [(33- | | | | | | | |
| | difluoropiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-25 | 4-(5-benzyl-3- | 337 | 47.9 | 200 | 194 | 49 | 2.5 | SF-A |
| | {[(3R4R)-3- | | | | | | | |
| | (hydroxymethyl)- | | | | | | | |
| | 34-dimethyl- | | | | | | | |
| | pyrrolidin-1- | | | | | | | |
| | yl]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-26 | 4-[5-benzyl-3- | 359 | 65.2 | 200 | 278 | 40 | 2.5 | SF-A |
| | ({3- | | | | | | | |
| | [(cyclopropyl- | | | | | | | |
| | methoxy)methyl] | | | | | | | |
| | pyrrolidin-1- | | | | | | | |
| | yl}methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-27 | 4-(5-benzyl-3- | 377 | 47.2 | 200 | 388 | 53 | 2.5 | SF-A |
| | {[bis(2- | | | | | | | |
| | methoxyethyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-28 | 4-{5-benzyl-3- | 389 | 46 | 200 | 212 | 55 | 2.5 | SF-A |
| | [(3- | | | | | | | |
| | cyanopiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-29 | methyl (2R)-1- | 394 | 47.7 | 200 | 332 | 49 | 2.5 | SF-A |
| | ({1-[4- | | | | | | | |
| | (aminosulfonyl)- | | | | | | | |
| | 2- | | | | | | | |
| | fluorophenyl]- | | | | | | | |
| | 5-benzyl-1H- | | | | | | | |
| | 124-triazol-3- | | | | | | | |
| | yl}methyl)piperidine- | | | | | | | |
| | 2- | | | | | | | |
| | carboxylate | | | | | | | |
| T-30 | 4-(5-benzyl-3- | 396 | 43.9 | 200 | 180 | 64 | 2.5 | SF-A |
| | {[4-(2- | | | | | | | |
| | hydroxyethyl) | | | | | | | |
| | piperidin-1- | | | | | | | |
| | yl]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-31 | methyl 1-({1-[4- | 396 | 46 | 200 | 317 | 50.5 | 2.5 | SF-A |
| | (aminosulfonyl)- | | | | | | | |
| | 2- | | | | | | | |
| | fluorophenyl]- | | | | | | | |
| | 5-benzyl-1H- | | | | | | | |
| | 124-triazol-3- | | | | | | | |
| | yl}methyl)-D- | | | | | | | |
| | prolinate | | | | | | | |
| T-32 | 4-{5-benzyl-3- | 418 | 52.8 | 200 | 161 | 38 | 2.5 | SF-A |
| | [(3- | | | | | | | |
| | propoxypiperidin- | | | | | | | |
| | 1-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-33 | 4-{5-benzyl-3- | 444 | 9.77 | 0.09 | 376 | 34 | 2.5 | SF-A |
| | [(6-hydroxy-14- | | | | | | | |
| | oxazepan-4- | | | | | | | |
| | yl)methyl]-1H- | | | | | | | |
| | 124-triazol-1- | | | | | | | |
| | yl}-3- | | | | | | | |
| | fluorobenzene- | | | | | | | |
| | sulfonamide | | | | | | | |
| T-34 | 4-(5-benzyl-3- | 499 | 38 | 200 | ND, | 37 | 2.5 | SF-A |
| | {[(7S8aS)-7- | | | | IC50 | | | |
| | fluorohexahydro- | | | | 3.26 nM | | | |
| | pyrrolo[12- | | | | | | | |
| | a]pyrazin- | | | | | | | |
| | 2(1H)- | | | | | | | |
| | yl]methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-35 | 4-(5-benzyl-3- | 153 | 8.65 | 0.09 | 57 | 84 | 2.5 | SF-A |
| | {[(2- | | | | | | | |
| | hydroxyethyl) | | | | | | | |
| | (isopropyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-36 | 4-(5-benzyl-3- | 175 | 9.02 | 0.09 | 186 | 49 | 2.5 | SF-A |
| | {[(2- | | | | | | | |
| | methoxyethyl) | | | | | | | |
| | (propyl)amino] | | | | | | | |
| | methyl}-1H-124- | | | | | | | |
| | triazol-1-yl)-3- | | | | | | | |
| | fluorobenzene- | | | | | | | |
| | sulfonamide | | | | | | | |
| T-37 | 4-[5-benzyl-3- | 264 | −5.65 | 0.09 | 129 | 52 | 2.5 | SF-A |
| | ({methyl[(5- | | | | | | | |
| | methyl-1H- | | | | | | | |
| | pyrazol-3- | | | | | | | |
| | yl)methyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-38 | 4-[5-benzyl-3- | 354 | 10.5 | 0.09 | 148 | 54 | 2.5 | SF-A |
| | ({methyl[2-(1H- | | | | | | | |
| | pyrazol-1- | | | | | | | |
| | yl)ethyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-39 | 4-(5-benzyl-3- | 409 | 4.84 | 0.09 | 143 | 65 | 2.5 | SF-A |
| | {[(3R)-3- | | | | | | | |
| | hydroxypiperidin- | | | | | | | |
| | 1-yl]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-40 | 4-{5-benzyl-3- | 428 | 18.8 | 0.09 | 438 | 49 | 2.5 | SF-A |
| | [(6-methoxy- | | | | | | | |
| | 14-oxazepan- | | | | | | | |
| | 4-yl)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-41 | 4-(5-benzyl-3- | 451 | 40.5 | 200 | 343 | 58 | 2.5 | SF-A |
| | {[(3R)-3- | | | | | | | |
| | methylmorpholin- | | | | | | | |
| | 4-yl]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-42 | 4-(5-benzyl-3- | 484 | 37.6 | 200 | ND, | 42 | 2.5 | SF-A |
| | {[methyl(propyl) | | | | 42% at | | | |
| | amino]methyl}- | | | | 2.5 nM | | | |
| | 1H-124- | | | | | | | |
| | triazol-1-yl)-3- | | | | | | | |
| | fluorobenzene- | | | | | | | |
| | sulfonamide | | | | | | | |
| T-43 | 4-[5-benzyl-3- | 93.8 | 10.9 | 0.09 | 106 | 41 | 2.5 | LC-D |
| | ({[(1R2S)-2- | | | | | | | |
| | hydroxy-1- | | | | | | | |
| | methyl-2- | | | | | | | |
| | phenylethyl] | | | | | | | |
| | amino}methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-44 | 4-[5-benzyl-3- | 105 | −3.48 | 0.09 | 159 | 49 | 2.5 | LC-D |
| | ({[(1S)-1- | | | | | | | |
| | benzyl-2- | | | | | | | |
| | hydroxyethyl] | | | | | | | |
| | amino}methyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| T-45 | 4-[5-benzyl-3- | 105 | 59.2 | 200 | 130 | 53 | 2.5 | LC-D |
| | ({[(1- | | | | | | | |
| | phenylcyclopropyl) | | | | | | | |
| | methyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-46 | 4-[5-benzyl-3- | 111 | 9.13 | 0.09 | 122 | 57 | 2.5 | LC-D |
| | ({[(2S)-2- | | | | | | | |
| | hydroxy-2- | | | | | | | |
| | phenylethyl] | | | | | | | |
| | amino}methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-47 | 4-[5-benzyl-3- | 119 | 56.4 | 200 | 109 | 41 | 2.5 | LC-D |
| | ({[(1S)-2- | | | | | | | |
| | methoxy-1- | | | | | | | |
| | phenylethyl] | | | | | | | |
| | amino}methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-48 | 4-[5-benzyl-3- | 135 | 62 | 200 | 114 | 50 | 2.5 | LC-D |
| | ({[2-(3- | | | | | | | |
| | fluorophenyl) | | | | | | | |
| | ethyl]amino}methyl)- | | | | | | | |
| | 1H-124- | | | | | | | |
| | triazol-1-yl]-3- | | | | | | | |
| | fluorobenzene- | | | | | | | |
| | sulfonamide | | | | | | | |
| T-49 | 4-(5-benzyl-3- | 146 | 48.2 | 200 | ND, | 49 | 2.5 | LC-D |
| | {[(4-fluoro-3- | | | | IC50 | | | |
| | methylbenzyl) | | | | 3.8 nM | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-50 | 4-[5-benzyl-3- | 155 | 4.35 | 0.09 | 23 | 63 | 2.5 | LC-D |
| | ({[(1S2R)-2- | | | | | | | |
| | hydroxy-1- | | | | | | | |
| | methyl-2- | | | | | | | |
| | phenylethyl] | | | | | | | |
| | amino}methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-51 | 4-(5-benzyl-3- | 156 | 47.8 | 200 | 220 | 39 | 2.5 | LC-D |
| | {[(cyclohexyl- | | | | | | | |
| | methyl)amino] | | | | | | | |
| | methyl}-1H-124- | | | | | | | |
| | triazol-1-yl)-3- | | | | | | | |
| | fluorobenzene- | | | | | | | |
| | sulfonamide | | | | | | | |
| T-52 | 4-[5-benzyl-3- | 162 | 54 | 200 | 180 | 39 | 2.5 | LC-D |
| | ({[(2S)-2- | | | | | | | |
| | phenylpropyl] | | | | | | | |
| | amino}methyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| T-53 | 4-[5-benzyl-3- | 165 | 7.83 | 0.09 | 66 | 75 | 2.5 | LC-D |
| | ({[(1S2R)-2- | | | | | | | |
| | hydroxycyclo- | | | | | | | |
| | hexyl]amino}methyl)- | | | | | | | |
| | 1H-124- | | | | | | | |
| | triazol-1-yl]-3- | | | | | | | |
| | fluorobenzene- | | | | | | | |
| | sulfonamide | | | | | | | |
| T-54 | 4-[5-benzyl-3- | 165 | 0.893 | 0.09 | 174 | 38 | 2.5 | LC-D |
| | ({[(2-(2- | | | | | | | |
| | fluorophenoxy) | | | | | | | |
| | ethyl]amino} | | | | | | | |
| | methyl)-1H-124- | | | | | | | |
| | triazol-1-yl]-3- | | | | | | | |
| | fluorobenzene- | | | | | | | |
| | sulfonamide | | | | | | | |
| T-55 | 4-(5-benzyl-3- | 166 | 50.5 | 200 | 42 | 59 | 2.5 | LC-D |
| | {[(1-isopropyl- | | No | | | | | |
| | 2- | | Data | | | | | |
| | methylpropyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-56 | 4-(5-benzyl-3- | 180 | 50.6 | 200 | 88 | 50 | 2.5 | LC-D |
| | {[(4- | | | | | | | |
| | fluorobenzyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-57 | 4-(5-benzyl-3- | 181 | 60 | 200 | 137 | 68 | 2.5 | LC-D |
| | [(cycloheptyl- | | | | | | | |
| | amino)methyl]- | | | | | | | |
| | 1H-124-triazol- | | | | | | | |
| | 1-yl}-3- | | | | | | | |
| | fluorobenzene- | | | | | | | |
| | sulfonamide | | | | | | | |
| T-58 | 4-[5-benzyl-3- | 186 | 54.1 | 200 | 114 | 51 | 2.5 | LC-D |
| | ({[2-(2- | | | | | | | |
| | fluorophenyl) | | | | | | | |
| | ethyl]amino}methyl)- | | | | | | | |
| | 1H-124- | | | | | | | |
| | triazol-1-yl]-3- | | | | | | | |
| | fluorobenzene- | | | | | | | |
| | sulfonamide | | | | | | | |
| T-59 | 4-(5-benzyl-3- | 187 | 50.5 | 200 | 336 | 53 | 2.5 | LC-D |
| | {[(3- | | | | | | | |
| | methylbutyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-60 | 4-(5-benzyl-3- | 197 | 49.7 | 200 | 66 | 57 | 2.5 | LC-D |
| | {[(25- | | | | | | | |
| | difluorobenzyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-61 | 4-(5-benzyl-3- | 202 | 59.6 | 200 | 137 | 40 | 2.5 | LC-D |
| | {[(2- | | | | | | | |
| | phenylethyl)amino] | | | | | | | |
| | methyl}-1H- | | | | | | | |
| | 124-triazol-1- | | | | | | | |
| | yl)-3- | | | | | | | |
| | fluorobenzene- | | | | | | | |
| | sulfonamide | | | | | | | |
| T-62 | 4-[5-benzyl-3- | 206 | 38 | 200 | ND, | 51 | 2.5 | LC-D |
| | ({[2-(4- | | | | 50% Inh | | | |
| | methoxyphenyl) | | | | @ | | | |
| | ethyl]amino}methyl)- | | | | 2.5 nM | | | |
| | 1H-124- | | | | | | | |
| | triazol-1-yl]-3- | | | | | | | |
| | fluorobenzene- | | | | | | | |
| | sulfonamide | | | | | | | |
| T-63 | 4-(5-benzyl-3- | 207 | 40.7 | 200 | 117 | 41 | 2.5 | LC-D |
| | {[(2- | | | | | | | |
| | ethoxybenzyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-64 | 4-(5-benzyl-3- | 219 | 5.8 | 0.09 | 162 | 47 | 2.5 | LC-D |
| | {[(2- | | | | | | | |
| | phenoxyethyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-65 | 4-[5-benzyl-3- | 220 | 46.6 | 200 | 87 | 58 | 2.5 | LC-D |
| | ({[1R)-1- | | | | | | | |
| | phenylethyl] | | | | | | | |
| | amino}methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-66 | 4-[5-benzyl-3- | 229 | 43.4 | 200 | 130 | 52 | 2.5 | LC-D |
| | ({[2-(3- | | | | | | | |
| | methylphenyl) | | | | | | | |
| | ethyl]amino} | | | | | | | |
| | methyl)-1H-124- | | | | | | | |
| | triazol-1-yl]-3- | | | | | | | |
| | fluorobenzene- | | | | | | | |
| | sulfonamide | | | | | | | |
| T-67 | 4-(5-benzyl-3- | 235 | 44.6 | 200 | 149 | 47 | 2.5 | LC-D |
| | {[2R)- | | | | | | | |
| | bicyclo[2.2.1] | | | | | | | |
| | hept-2- | | | | | | | |
| | ylamino]methyl}- | | | | | | | |
| | 1H-124- | | | | | | | |
| | triazol-1-yl)-3- | | | | | | | |
| | fluorobenzene- | | | | | | | |
| | sulfonamide | | | | | | | |
| T-68 | 4-[5-benzyl-3- | 238 | 3.57 | 0.09 | 255 | 55 | 2.5 | LC-D |
| | ({[2-(3-methyl- | | | | | | | |
| | 1H-pyrazol-1- | | | | | | | |
| | yl)ethyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-69 | 4-{5-benzyl-3- | 244 | 43 | 200 | 124 | 68 | 2.5 | LC-D |
| | [(cyclohexyl- | | | | | | | |
| | amino)methyl]-1H- | | | | | | | |
| | 124-triazol-1- | | | | | | | |
| | yl}-3- | | | | | | | |
| | fluorobenzene- | | | | | | | |
| | sulfonamide | | | | | | | |
| T-70 | 4-[5-benzyl-3- | 249 | 48.9 | 200 | 42 | 89 | 2.5 | LC-D |
| | ({[1- | | | | | | | |
| | (methoxymethyl) | | | | | | | |
| | propyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-71 | 4-[5-benzyl-3- | 257 | 47.8 | 200 | 136 | 47 | 2.5 | LC-D |
| | ({[(1R2S)-2- | | | | | | | |
| | (methoxymethyl) | | | | | | | |
| | cyclopentyl] | | | | | | | |
| | amino}methyl)- | | | | | | | |
| | 1H-124-triazol- | | | | | | | |
| | 1-yl]-3- | | | | | | | |
| | fluorobenzene- | | | | | | | |
| | sulfonamide | | | | | | | |
| T-72 | 4-[5-benzyl-3- | 265 | 43.8 | 200 | 119 | 70 | 2.5 | LC-D |
| | ({[1-methyl-2- | | | | | | | |
| | (1H-pyrazol-1- | | | | | | | |
| | yl)ethyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-73 | 4-[5-benzyl-3- | 272 | 42.7 | 200 | ND, | 50 | 2.5 | LC-D |
| | ({[2-(3- | | | | 50% at | | | |
| | methoxyphenyl) | | | | 2.5 nM | | | |
| | ethyl]amino} | | | | | | | |
| | methyl)-1H-124- | | | | | | | |
| | triazol-1-yl]-3- | | | | | | | |
| | fluorobenzene- | | | | | | | |
| | sulfonamide | | | | | | | |
| T-74 | 4-(5-benzyl-3- | 276 | 42.2 | 200 | 88 | 45 | 2.5 | LC-D |
| | {[(2- | | | | | | | |
| | fluorobenzyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-75 | 4-(5-benzyl-3- | 277 | 43.4 | 200 | 128 | 51 | 2.5 | LC-D |
| | {[(1- | | | | | | | |
| | phenylpropyl) | | | | | | | |
| | amino]methyl}- | | | | | | | |
| | 1H-124-triazol- | | | | | | | |
| | 1-yl)-3- | | | | | | | |
| | fluorobenzene- | | | | | | | |
| | sulfonamide | | | | | | | |
| T-76 | 4-{5-benzyl-3- | 285 | 39.9 | 200 | ND, | 41 | 2.5 | LC-D |
| | [(23-dihydro- | | | | 41% at | | | |
| | 1H-inden-2- | | | | 2.5 nM | | | |
| | ylamino)methyl]- | | | | | | | |
| | 1H-124- | | | | | | | |
| | triazol-1-yl}-3- | | | | | | | |
| | fluorobenzene- | | | | | | | |
| | sulfonamide | | | | | | | |
| T-77 | 4-[5-benzyl-3- | 299 | 40.7 | 200 | 177 | 50 | 2.5 | LC-D |
| | ({[(3S4R)-4- | | | | | | | |
| | cyclopropyl- | | | | | | | |
| | tetrahydrofuran-3- | | | | | | | |
| | yl]amino}methyl)- | | | | | | | |
| | 1H-124- | | | | | | | |
| | triazol-1-yl]-3- | | | | | | | |
| | fluorobenzene- | | | | | | | |
| | sulfonamide | | | | | | | |
| T-78 | 4-[5-benzyl-3- | 304 | 42.2 | 200 | 90 | 71 | 2.5 | LC-D |
| | ({[1-methyl-2- | | | | | | | |
| | (6- | | | | | | | |
| | methylpyridin- | | | | | | | |
| | 2- | | | | | | | |
| | yl)ethyl]amino} | | | | | | | |
| | methyl)-1H- | | | | | | | |
| | 124-triazol-1- | | | | | | | |
| | yl]-3- | | | | | | | |
| | fluorobenzene- | | | | | | | |
| | sulfonamide | | | | | | | |
| T-79 | 4-(5-benzyl-3- | 369 | 42.2 | 200 | 232 | 44 | 2.5 | LC-D |
| | {[(2- | | | | | | | |
| | isopropoxyethyl) | | | | | | | |
| | amino]methyl}- | | | | | | | |
| | 1H-124- | | | | | | | |
| | triazol-1-yl)-3- | | | | | | | |
| | fluorobenzene- | | | | | | | |
| | sulfonamide | | | | | | | |
| T-80 | 4-[5-benzyl-3- | 416 | 42.3 | 200 | 84 | 73 | 2.5 | LC-D |
| | ({[(1S)-2- | | | | | | | |
| | methoxy-1- | | | | | | | |
| | methylethyl] | | | | | | | |
| | amino}methyl)-1H- | | | | | | | |
| | 124-triazol-1- | | | | | | | |
| | yl]-3- | | | | | | | |
| | fluorobenzene- | | | | | | | |
| | sulfonamide | | | | | | | |

| Example # | Structure | 1H NMR | comments |
|---|---|---|---|
| U-292 | 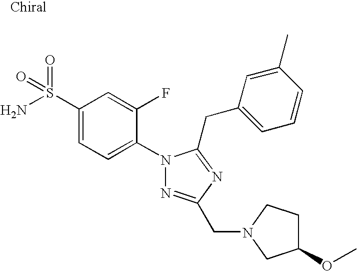 | 1H NMR (500 MHz, DMSO-D6) d ppm 1.50-1.64 (m, 1 H) 1.85-1.95 (m, 1 H) 2.12 (s, 3 H) 2.57-2.63 (m, J = 10.99 Hz, 1 H) 2.72 (dd, J = 10.30, 6.18 Hz, 2 H) 3.09 (s, 3 H) 3.58 (s, 2 H) 3.78-3.83 (m, 1 H) 3.97 (s, 2 H) 6.71-6.75 (m, 2 H) 6.91- 6.95 (m, 1 H) 7.04 (t, J = 7.69 Hz, 1 H) 7.67- 7.76 (m, 3 H) | |
| U-290 | 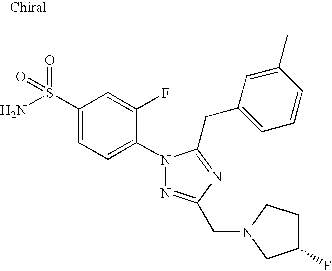 | 1H NMR (500 MHz, DMSO-D6) d ppm 1.73-1.87 (m, 1 H) 1.99-2.10 (m, 1 H) 2.12 (s, 3 H) 2.63-2.74 (m, 2 H) 3.62- 3.66 (m, 2 H) 3.98 (s, 2 H) 5.04-5.21 (m, 1 H) 6.69-6.76 (m, 2 H) 6.93 (d, J = 7.42 Hz, 1 H) 7.04 (t, J = 7.55 Hz, 1 H) 7.67- 7.78 (m, 3 H) | |
| U-281 | 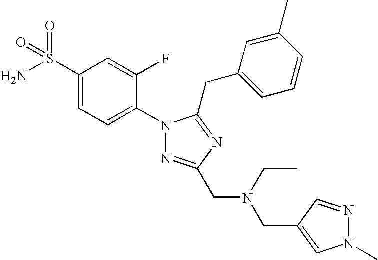 | 1H NMR (500 MHz, DMSO-D6) d ppm 1.03 (t, J = 7.00, 7.00 Hz, 3 H) 2.18 (s, 3 H) 2.15-2.20 (m, 3 H) 2.43-2.48 (m, 2 H) 3.78 (s, 3 H) 4.05 (s, 2 H) 6.79 (d, J = 7.42 Hz, 2 H) 6.99 (d, J = 7.42 Hz, 1 H) 7.10 (t, J = 7.42 Hz, 1 H) 7.32 (s, 1 H) 7.54 (s, 1H) 7.69-7.87 (m, 3 H) | |
| U-298 | 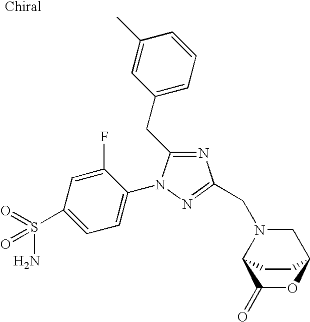 | 1H NMR (500 MHz, DMSO-d6) d ppm 1.63-1.79 (m, 3 H) 2.02-2.09 (m, 1 H) 2.13 (s, 3 H) 2.76-2.84 (m, 1 H) 3.09 (d, J = 11.26 Hz, 1 H) 3.58 (d, J = 13.46 Hz, 1 H) 3.79 (d, J = 13.46 Hz, 1 H) 3.99 (s, 2 H) 4.69 (s, 1 H) 6.71-6.77 (m, 2 H) 6.94 (d, J = 7.69 Hz, 1 H) 7.04 (t, J = 7.69 Hz, 1 H) 7.67-7.78 (m, 3 H) | |
| U-282 | 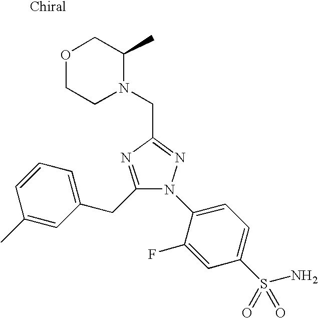 | 1H NMR (500 MHz, DMSO-D6) d ppm 0.95 (d, J = 6.32 Hz, 3 H) 2.11 (s, 3 H) 2.58- 2.67 (m, 2 H) 3.03 (t, 1 H) 3.59-3.63 (m, 2 H) 3.66 (d, J = 12.00 Hz, 1 H) 3.74 (d, J = 12.00 Hz, 1 H) 3.99 (s, 2 H) 6.66-6.74 (m, 2 H) 6.92 (d, J = 7.60 Hz, 1 H) 7.03 (t, J = 7.60 Hz, 1 H) 7.66-7.76 (m, 3 H) | |
| U-273 | 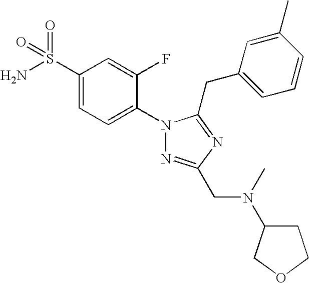 | 1H NMR (500 MHz, DMSO-d6) d ppm 1.68-1.76 (m, 1 H) 1.91-2.01 (m, 1 H) 2.13 (s, 3 H) 2.16 (s, 3 H) 3.06-3.12 (m, 1 H) 3.55-3.60 (m, 2 H) 3.69-3.79 (m, 2 H) 4.00 (s, 2 H) 6.70-6.76 (m, 2 H) 6.94 (d, J = 7.42 Hz, 1 H) 7.04 (t, J = 7.42 Hz, 1 H) 7.68-7.77 (m, 3 H) | |
| U-289 | 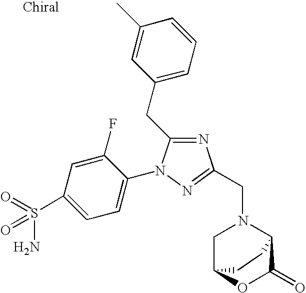 | 1H NMR (500 MHz, DMSO-d6) d ppm 1.63-1.79 (m, 3 H) 2.02-2.09 (m, 1 H) 2.13 (s, 3 H) 2.76-2.84 (m, 1 H) 3.09 (d, J = 11.26 Hz, 1 H) 3.58 (d, J = 13.46 Hz, 1 H) 3.79 (d, J = 13.46 Hz, 1 H) 3.99 (s, 2 H) 4.69 (s, 1 H) 6.71-6.77 (m, 2 H) 6.94 (d, J = 7.69 Hz, 1 H) 7.04 (t, J = 7.69 Hz, 1 H) 7.67-7.78 (m, 3 H) | |
| U-286 | 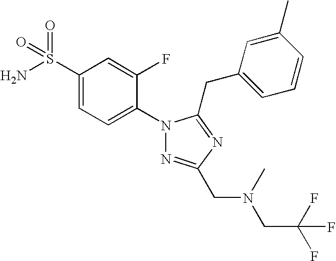 | 1H NMR (500 MHz, DMSO-d6) d ppm 2.13 (s, 3 H) 2.55 (s, 3 H) 3.25-3.32 (m, 2 H) 3.77 (s, 2 H) 4.01 (s, 2 H) 6.70-6.76 (m, 2 H) 6.94 (d, J = 7.14 Hz, 1 H) 7.04 (t, J = 7.14 Hz, 1 H) 7.58-7.65 (m, 1 H) 7.68- 7.78 (m, 3 H) | |
| U-284 | 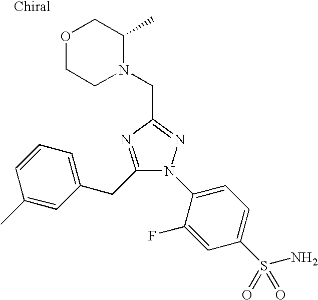 | 1H NMR (500 MHz, DMSO-D6) d ppm 0.95 (d, J = 6.32 Hz, 3 H) 2.11 (s, 3 H) 2.58- 2.67 (m, 2 H) 3.03 (t, 1 H) 3.59-3.63 (m, 2 H) 3.66 (d, J = 12.00 Hz, 1 H) 3.74 (d, J = 12.00 Hz, 1 H) 3.99 (s, 2 H) 6.68-6.74 (m, 2 H) 6.92 (d, J = 7.60 Hz, 1 H) 7.03 (t, J = 7.60 Hz, 1 H) 7.66-7.76 (m, 3 H) | |
| U-276 | 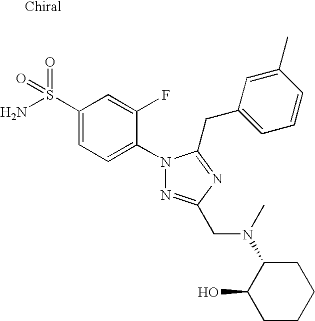 | 1H NMR (500 MHz, DMSO-d6) d ppm 1.02-1.18 (m, 4 H) 1.51-1.68 (m, 3 H) 1.82-1.89 (m, 1 H) 2.13 (s, 3 H) 2.21- 2.27 (m, 1 H) 2.24 (s, 3 H) 3.59 (d, J = 14.01 Hz, 1 H) 3.74 (d, J = 14.01 Hz, 1 H) 3.99 (s, 2 H) 4.11-4.16 (m, 1 H) 6.70-6.76 (m, 2 H) 6.93 (d, J = 7.42 Hz, 1 H) 7.04 (t, J = 7.42 Hz, 1 H) 7.66-7.77 (m, 3 H) | |
| U-312 | 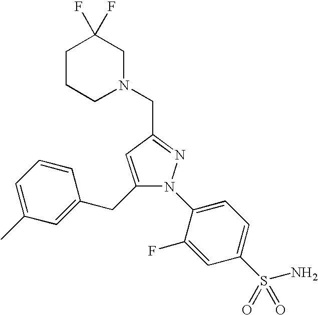 | 1H NMR (500 MHz, DMSO-D6) d ppm 1.54-1.63 (m, 2 H) 1.72-1.86 (m, 2 H) 2.12 (s, 3 H) 3.81 (s, 2 H) 6.12 (s, 1 H) 6.66-6.75 (m, 2 H) 6.92 (d, J = 7.42 Hz, 1 H) 7.04 (t, J = 7.42 Hz, 1 H) 7.54-7.62 (m, 3 H) 7.66-7.74 (m, 2 H) (Only observed peaks are described.) | SONH2 was observed. |
| U-345 | 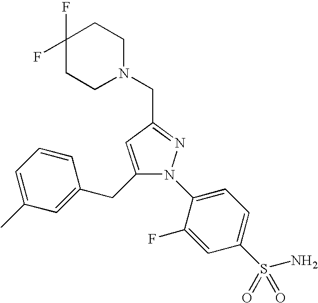 | 1H NMR (500 MHz, DMSO-D6) d ppm 1.93-2.06 (m, 4 H) 2.14 (s, 3 H) 3.83 (s, 2 H) 6.23 (s, 1 H) 6.65-6.81 (m, 2 H) 6.93 (d, J = 7.42 Hz, 1 H) 7.06 (t, J = 7.42 Hz, 1 H) 7.55-7.65 (m, 3 H) 7.68-7.76 (m, 2 H) (Only observed peak are described.) | SONH2 was observed. |

| | | Observed | | | | |
|---|---|---|---|---|---|---|
| Example # | Structure | Mass | MW | Synthetic Method | Protocol | IUPAC name |
| JJ J-1 | 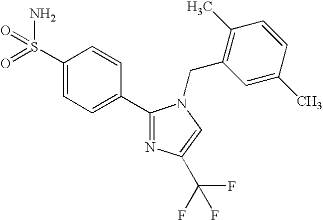 | 410.1 | 409.1 | L D | LJ0542 | 4-[1-(25- dimethylbenzyl)-4- (trifluoromethyl)-1H- imidazol-2- yl]benzenesulfonamide |
| JJ J-2 | 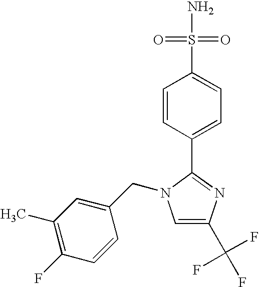 | 414.1 | 413.1 | L D | LJ0542 | 4-[1-(4-fluoro-3- methylbenzyl)-4- (trifluoromethyl)-1H- imidazol-2- yl]benzenesulfonamide |
| JJ J-3 | 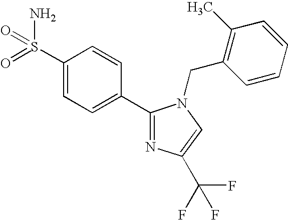 | 396.1 | 395.1 | L D | LJ0542 | 4-[1-(2- methylbenzyl)-4- (trifluoromethyl)-1H- imidazol-2- yl]benzenesulfonamide |
| JJ J-4 | 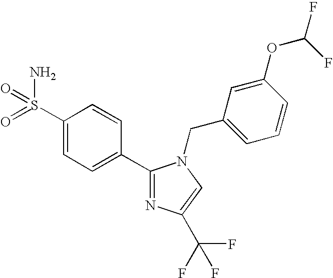 | 448.1 | 447.1 | L D | LJ0542 | 4-{1-[3- (difluoromethoxy) benzyl]-4- (trifluoromethyl)-1H- imidazol-2- yl]benzenesulfonamide |

| Example | CA-IV | CA-IV: % Inh @ | CA-IV: Min | CA-II | CAII % Inh @ | CA II: | Purification |
|---|---|---|---|---|---|---|---|
| # | IC50 (nM) | Min dose | dose (nM) | Kd (pM) | Min dose | Min dose (nM) | Method |
| JJ | 130 | 56 | 200 | 608 | 46 | 2.5 | SF-B |
| J-1 | | | | | | | |
| JJ | 309 | 45 | 200 | 552 | 37 | 2.5 | SF-B |
| J-2 | | | | | | | |
| JJ | 346 | 36 | 200 | 369 | 47 | 2.5 | SF-B |
| J-3 | | | | | | | |
| JJ | 440 | 29 | 200 | 1,390 | 39 | 2.5 | SF-B |
| J-4 | | | | | | | |

| Example | | | |
|---|---|---|---|
| # | Structure | 1H NMR | comments |
| U-292 | 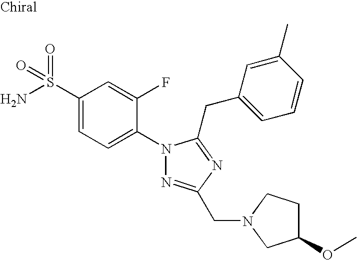 | 1H NMR (500 MHz, DMSO-D6) d ppm 1.50-1.64 (m, 1 H) 1.85-1.95 (m, 1 H) 2.12 (s, 3 H) 2.57-2.63 (m, J = 10.99 Hz, 1 H) 2.72 (dd, J = 10.30, 6.18 Hz, 2 H) 3.09 (s, 3 H) 3.58 (s, 2 H) 3.78-3.83 (m, 1 H) 3.97 (s, 2 H) 6.71-6.75 (m, 2 H) 6.91- 6.95 (m, 1 H) 7.04 (t, J = 7.69 Hz, 1 H) 7.67- 7.76 (m, 3 H) | |
| U-290 | 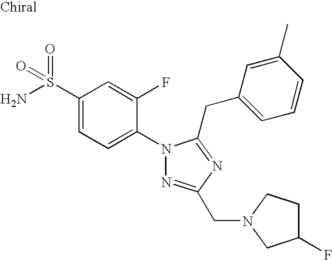 | 1H NMR (500 MHz, DMSO-D6) d ppm 1.73-1.87 (m, 1 H) 1.99-2.10 (m, 1 H) 2.12 (s, 3 H) 2.63-2.74 (m, 2 H) 3.62- 3.66 (m, 2 H) 3.98 (s, 2 H) 5.04-5.21 (m, 1 H) 6.69-6.76 (m, 2 H) 6.93 (d, J = 7.42 Hz, 1 H) 7.04 (t, J = 7.55 Hz, 1 H) 7.67- 7.78 (m, 3 H) | |
| U-281 | 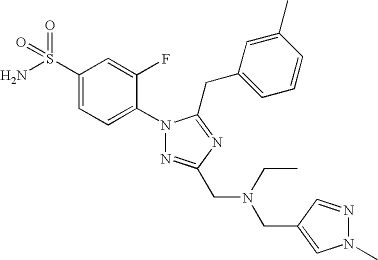 | 1H NMR (500 MHz, DMSO-D6) d ppm 1.03 (t, J = 7.00, 7.00 Hz, 3 H) 2.18 (s, 3 H) 2.15-2.20 (m, 3 H) 2.43-2.48 (m, 2 H) 3.78 (s, 3 H) 4.05 (s, 2 H) 6.79 (d, J = 7.42 Hz, 2 H) 6.99 (d, J = 7.42 Hz, 1 H) 7.10 (t, J = 7.42 Hz, 1 H) 7.32 (s, 1 H) 7.54 (s, 1 H) 7.69-7.87 (m, 3 H) | |
| U-298 | 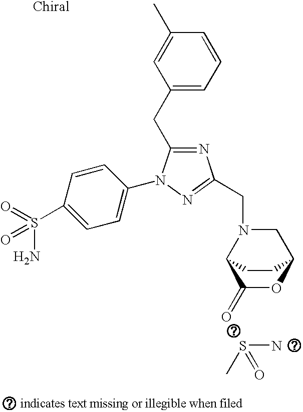 | 1H NMR (500 MHz, DMSO-d6) d ppm 1.63-1.79 (m, 3 H) 2.02-2.09 (m, 1 H) 2.13 (s, 3 H) 2.76-2.84 (m, 1 H) 3.09 (d, J = 11.26 Hz, 1 H) 3.58 (d, J = 13.46 Hz, 1 H) 3.79 (d, J = 13.46 Hz, 1 H) 3.99 (s, 2 H) 4.69 (s, 1 H) 6.71-6.77 (m, 2 H) 6.94 (d, J = 7.69 Hz, 1 H) 7.04 (t, J = 7.69 Hz, 1 H) 7.67-7.78 (m, 3 H) | |
| U-282 | 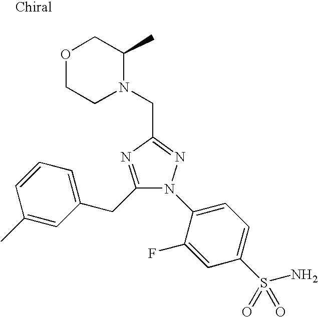 | 1H NMR (500 MHz, DMSO-D6) d ppm 0.95 (d, J = 6.32 Hz, 3 H) 2.11 (s, 3 H) 2.58- 2.67 (m, 2 H) 3.03 (t, 1 H) 3.59-3.63 (m, 2 H) 3.66 (d, J = 12.00 Hz, 1 H) 3.74 (d, J = 12.00 Hz, 1 H) 3.99 (s, 2 H) 6.68-6.74 (m, 2 H) 6.92 (d, J = 7.60 Hz, 1 H) 7.03 (t, J = 7.60 Hz, 1 H) 7.66-7.76 (m, 3 H) | |
| U-273 | 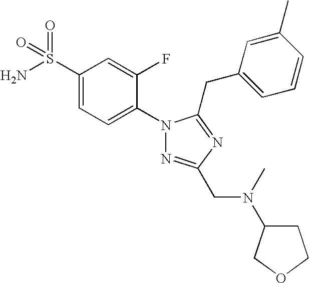 | 1H NMR (500 MHz, DMSO-d6) d ppm 1.68-1.76 (m, 1 H) 1.91-2.01 (m, 1 H) 2.13 (s, 3 H) 2.16 (s, 3 H) 3.06-3.12 (m, 1 H) 3.55-3.60 (m, 2 H) 3.69-3.79 (m, 2 H) 4.00 (s, 2 H) 6.70-6.76 (m, 2 H) 6.94 (d, J = 7.42 Hz, 1 H) 7.04 (t, J = 7.42 Hz, 1 H) 7.68-7.77 (m, 3 H) | |
| U-289 | 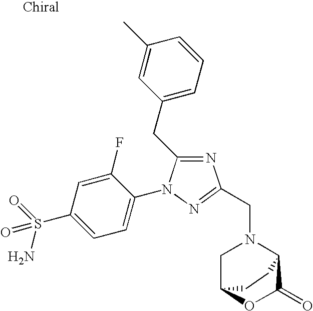 | 1H NMR (500 MHz, DMSO-d6) d ppm 1.63-1.79 (m, 3 H) 2.02-2.09 (m, 1 H) 2.13 (s, 3 H) 2.76-2.84 (m, 1 H) 3.09 (d, J = 11.26 Hz, 1 H) 3.58 (d, J = 13.46 Hz, 1 H) 3.79 (d, J = 13.46 Hz, 1 H) 3.99 (s, 2 H) 4.69 (s, 1 H) 6.71-6.77 (m, 2 H) 6.94 (d, J = 7.69 Hz, 1 H) 7.04 (t, J = 7.69 Hz, 1 H) 7.67-7.78 (m, 3 H) | |
| U-286 | 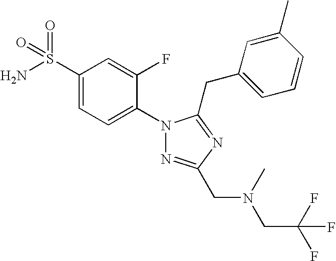 | 1H NMR (500 MHz, DMSO-d6) d ppm 2.13 (s, 3 H) 2.55 (s, 3H) 3.25-3.32 (m, 2 H) 3.77 (s, 2 H) 4.01 (s, 2 H) 6.70-6.76 (m, 2 H) 6.94 (d, J = 7.14 Hz, 1 H) 7.04 (t, J = 7.14 Hz, 1 H) 7.58-7.65 (m, 1 H) 7.68- 7.78 (m, 3 H) | |
| U-284 | 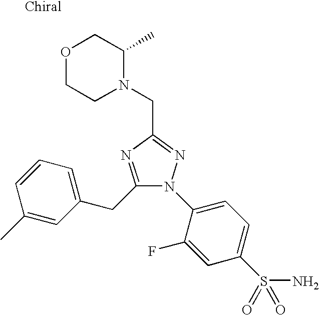 | 1H NMR (500 MHz, DMSO-D6) d ppm 0.95 (d, J = 6.32 Hz, 3 H) 2.11 (s, 3 H) 2.58- 2.67 (m, 2 H) 3.03 (t, 1 H) 3.59-3.63 (m, 2 H) 3.66 (d, J = 12.00 Hz, 1 H) 3.74 (d, J = 12.00 Hz, 1 H) 3.99 (s, 2 H) 6.68-6.74 (m, 2 H) 6.92 (d, J = 7.60 Hz, 1 H) 7.03 (t, J = 7.60 Hz, 1 H) 7.66-7.76 (m, 3 H) | |
| U-276 | 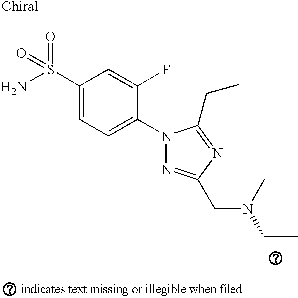 | 1H NMR (500 MHz, DMSO-d6) d ppm 1.02-1.18 (m, 4 H) 1.51-1.68 (m, 3 H) 1.82-1.89 (m, 1 H) 2.13 (s, 3 H) 2.21- 2.27 (m, 1 H) 2.24 (s, 3 H) 3.59 (d, J = 14.01 Hz, 1 H) 3.74 (d, J = 14.01 Hz, 1 H) 3.99 (s, 2 H) 4.11-4.16 (m, 1 H) 6.70-6.76 (m, 2 H) 6.93 (d, J = 7.42 Hz, 1 H) 7.04 (t, J = 7.42 Hz, 1 H) 7.66-7.77 (m, 3 H) | |
| U-312 | 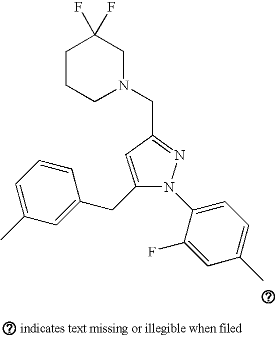 | 1H NMR (500 MHz, DMSO-D6) d ppm 1.54-1.63 (m, 2 H) 1.72-1.86 (m, 2 H) 2.12 (s, 3 H) 3.61 (s, 2 H) 6.12 (s, 1 H) 6.66-6.75 (m, 2 H) 6.92 (d, J = 7.42 Hz, 1 H) 7.04 (t, J = 7.42 Hz, 1 H) 7.54-7.62 (m, 3 H) 7.66-7.74 (m, 2 H) (Only observed peaks are described.) | SONH2 was observed. |
| U-345 | 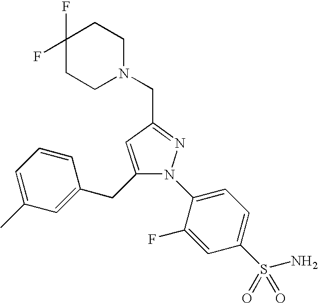 | 1H NMR (500 MHz, DMSO-D6) d ppm 1.93-2.08 (m, 4 H) 2.14 (s, 3 H) 3.83 (s, 2 H) 6.23 (s, 1 H) 6.65-6.81 (m, 2 H) 6.93 (d, J = 7.42 Hz, 1 H) 7.06 (t, J = 7.42 Hz, 1 H) 7.55-7.65 (m, 3 H) 7.68-7.76 (m, 2 H) (Only observed peak are described.) | SONH2 was observed. |

TABLE
Examples assayed with CIAX.

| | | CAIX IC50 |
|---|---|---|
| Example number | Structure | (nM) |
| | 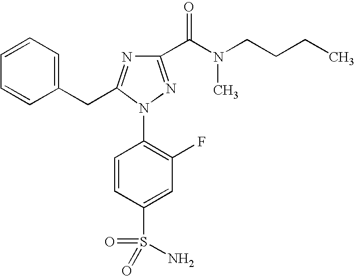 | 2.79 |
| A-1d | 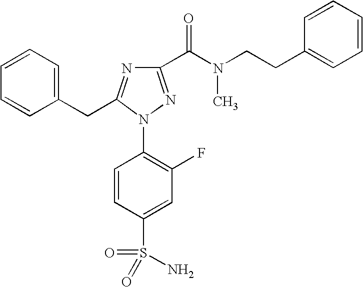 | 2.94 |
| F-1ak | 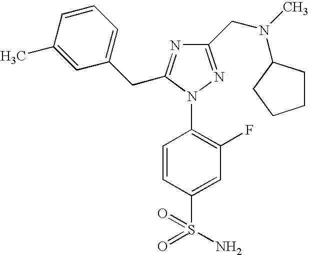 | 3.83 |
| F-1aj | 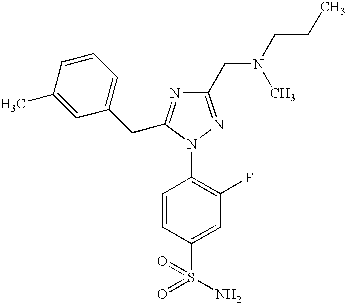 | 4.32 |
| QQ-10 | 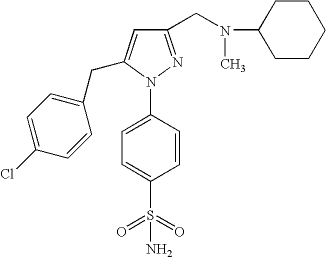 | 5.18 |
| A-1j | 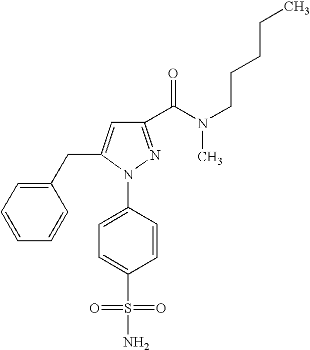 | 6.44 |
| G-1 | 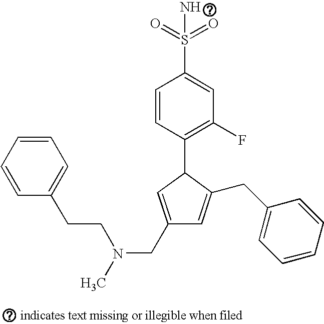 | 7.1 |
| F-1z | 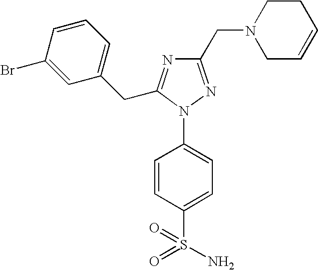 | 7.99 |
| f-3g | 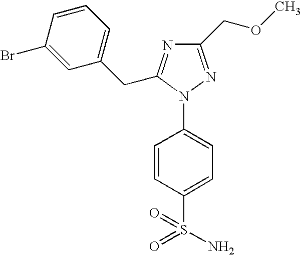 | 8.11 |
| G-1b | 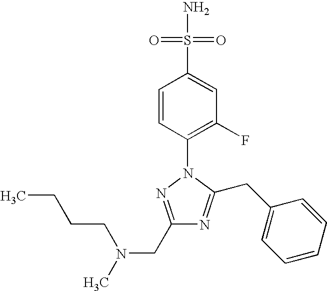 | 9.01 |
| F-1aq | 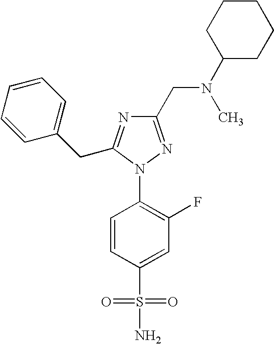 | 9.41 |
| qq-3a | 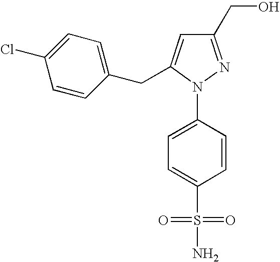 | 13.45 |
| F-1 | 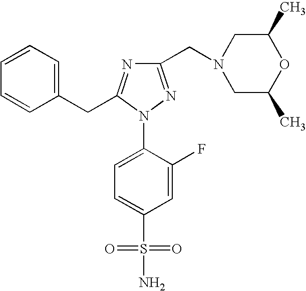 | 26.08 |
| L-1a | 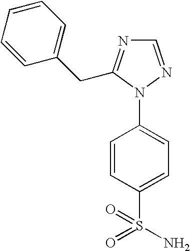 | 35.63 |
| f-3a | 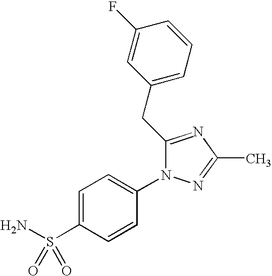 | 36.89 |
| CCC-1a | 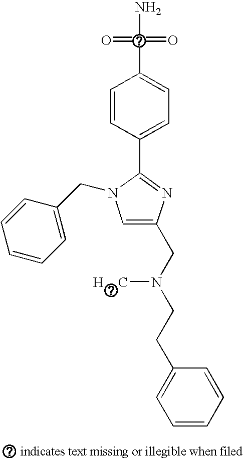 | 39.33 |
| R-21 | 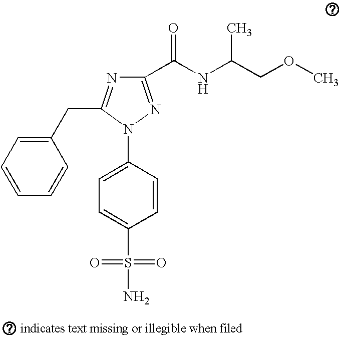 | 53.27 |

TABLE
Exdamples assayed with CAXII.

| | example | | CAXII IC 50 | % inh CAXII |
|---|---|---|---|---|
| Compound ID | number | Mol. Structure | (nM) | @ 1 uM |
| Acetazolamide | | 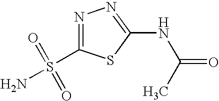 | 42.2 | ND |
| Ethoxzolamide | | 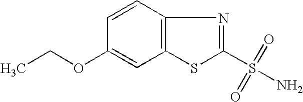 | 17.2 | ND |
| Dorzolamide | | 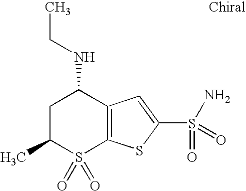 | 13.7 | ND |
| Brinzolamide | | 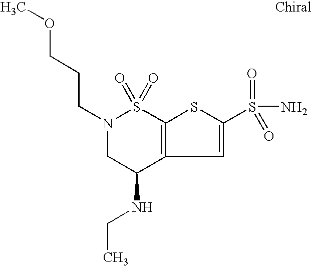 | 17.4 | ND |
| | GG-1 | 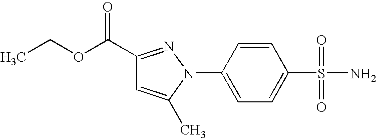 | 896 | 51 |
| | II-1b | 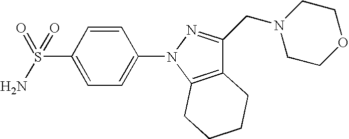 | 119 | 89 |
| | EE-1g | 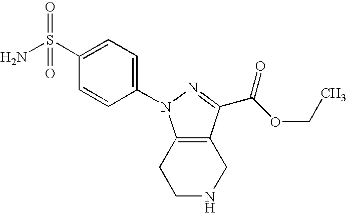 | >1000 | 31 |
| | MM-1c | 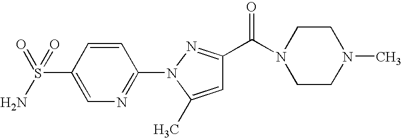 | 166 | 86 |
| | MM-1b | 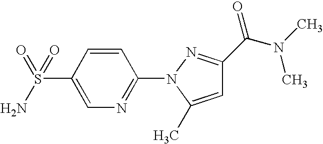 | 66.5 | 96 |
| | a-4 | 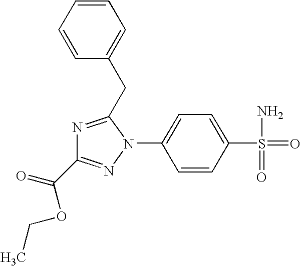 | 73.8 | 95 |
| | I-5b | 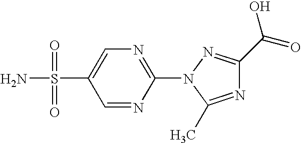 | >1000 | 46 |
| | a-5 | 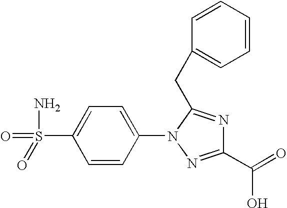 | 25.8 | 99 |

1. A compound having formula I: 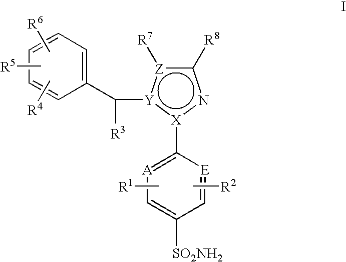
or a pharmaceutically acceptable salt or solvate thereof, wherein:
A and E are each independently C or N;
X is C; Y is N; and Z is C; or
X is N; Y is C; and Z is C or N;
R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O) NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁷ and R⁸ are optionally each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-(CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), or
R⁷ and R⁸ form a fused 6-membered heteroaryl ring;
R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜO(CH₂)ᵤ(C₁-C₁₂)alkyl, (CH₂)ₜCN, (CH₂)ₜCF₃, (CF₂)ₜCF₃, (CH₂)ₜOCF₃, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁰ and R¹¹ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹²R¹³, (CH₂)ₜNR¹²R¹³, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹² and R¹³ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NH₂, (CH₂)ₜC(═O)NH(C₁-C₁₂)alkyl, (CH₂)ₜC(═O)N((C₁-C₁₂)alkyl)₂, (CH₂)ₜN H₂, (CH₂)ₜNH(C₁-C₁₂)alkyl, (CH₂)ₜN((C₁-C₁₂)alkyl)₂, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹, R², R³, R⁴, R⁵, R⁶, R⁷, R⁸, R⁹, R¹⁰, R¹¹, R¹², R¹³, R¹⁴ and R¹⁵ groups are each optionally independently substituted with 1 to 5 R¹⁶ groups;
R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)(C₆-C₁₀)aryl, (CH₂)ₜ(3-10 membered heterocyclyl) or (CH₂)ₜC(═O)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) and (CH₂)ₜC(═O)(CH₂)ᵤ(3-10 membered heterocyclyl);
t, u and v are each independently 0, 1, 2, 3, 4, 5 or 6; and
w is 1, 2 or 3.

2. The compound of claim 1, wherein:
A and E are each independently C or N;
X is C; Y is N; and Z is C; or
X is N; Y is C; and Z is C or N;
R¹ and R² are each independently H, F, Cl, Br, I or (C₁-C₆)alkyl;
R³ is H, F, Cl, Br, I or (C₁-C₆)alkyl;
R⁴, R⁵ and R⁶ are each independently H, F, Cl, Br, I, (C₁-C₆)alkyl, CF₃ or OCHF₂;
R⁷ is optionally H or (C₁-C₆)alkyl; and
R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or
R⁷ and R⁸ form a fused pyridinyl ring.

3. The compound of claim 1, wherein:
A and E are each independently C or N;
X is C; Y is N; and Z is C;
R¹ and R² are each independently H, F, Cl, Br, I or (C₁-C₆)alkyl;
R³ is H, F, Cl, Br, I or (C₁-C₆)alkyl;
R⁴, R⁵ and R⁶ are each independently H, F, Cl, Br, I, (C₁-C₆)alkyl, CF₃ or OCHF₂;
R⁷ is optionally H or (C₁-C₆)alkyl; and
R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or
R⁷ and R⁸ form a fused pyridinyl ring.

4. The compound of claim 1, having formula II: 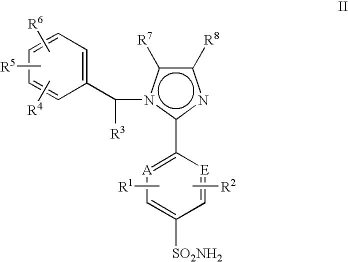
or a pharmaceutically acceptable salt or solvate thereof, wherein:
A and E are each independently C or N;
R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁷ and R⁸ are optionally each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), or
R⁷ and R⁸ form a fused 6-membered heteroaryl ring;
R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜO(CH₂)ᵤ(C₁-C₁₂)alkyl, (CH₂)ₜCN, (CH₂)ₜCF₃, (CF₂)ₜCF₃, (CH₂)ₜOCF₃, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁰ and R¹¹ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)C(═O)R⁹, (CH₂)ₜOR⁹, (CH₂)ₜSO₂R⁹, (CH₂)ₜC(═O)NR¹²R¹³, (CH₂)ₜNR¹²R¹³, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹² and R¹³ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NH₂, (CH₂)ₜC(═O)NH(C₁-C₁₂)alkyl, (CH₂)ₜC(═O)N((C₁-C₁₂)alkyl)₂, (CH₂)ₜNH₂, (CH₂)ₜNH(C₁-C₁₂)alkyl, (CH₂)ₜN((C₁-C₁₂)alkyl)₂, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹, R², R³, R⁴, R⁵, R⁶, R⁶, R⁷, R⁸, R⁹, R¹⁰, R¹¹, R¹², R¹³, R¹⁴ and R¹⁵ groups are each optionally independently substituted with 1 to 5 R¹⁶ groups;
R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) and (CH₂)ₜC(═O)(CH₂)ᵤ(3-10 membered heterocyclyl);
t, u and v are each independently 0, 1, 2, 3, 4, 5 or 6; and
w is 1, 2 or 3.

5. The compound of claim 4, wherein:
A and E are each independently C or N;
R¹ and R² are each independently H, F, Cl, Br, I or (C₁-C₆)alkyl;
R³ is H, F, Cl, Br, I or (C₁-C₆)alkyl;
R⁴, R⁵ and R⁶ are each independently H, F, Cl, Br, I, (C₁-C₆)alkyl, CF₃ or OCHF₂;
R⁷ is H or (C₁-C₆)alkyl; and
R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl); or
R⁷ and R⁸ form a fused pyridinyl ring.

6. The compound of claim 1, having formula III: 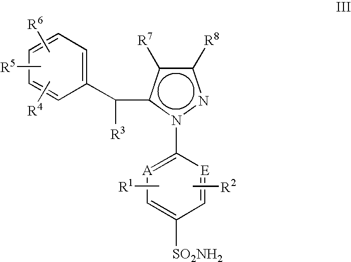
or a pharmaceutically acceptable salt or solvate thereof, wherein:
A and E are each independently C or N;
R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl , (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O) NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁷ and R⁸ are optionally each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), or
R⁷ and R⁸ form a fused 6-membered heteroaryl ring;
R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜO(CH₂)ᵤ(C₁-C₁₂)alkyl, (CH₂)ₜCN, (CH₂)ₜCF₃, (CF₂)ₜCF₃, (CH₂)ₜOCF₃, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁰ and R¹¹ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹²R¹³, (CH₂)₆NR¹²R¹³, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹² and R¹³ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C ₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NH₂, (CH₂)ₜC(═O)NH(C₁-C₁₂)alkyl, (CH₂)ₜC(═O)N((C₁-C₁₂)alkyl)₂, (CH₂)ₜNH₂, (CH₂)ₜNH(C₁-C₁₂)alkyl, (CH₂)ₜN((C₁-C₁₂)alkyl)₂, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹, R², R³, R⁴, R⁵, R⁶, R⁷, R⁸, R⁹, R¹⁰, R¹¹, R¹², R¹³, R¹⁴ and R¹⁵ groups are each optionally independently substituted with 1 to 5 R¹⁶ groups;
R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) and (CH₂)ₜC(═O)(CH₂)ᵤ(3-10 membered heterocyclyl);
t, u and v are each independently 0, 1, 2, 3, 4, 5 or 6; and
w is 1, 2 or 3.

7. The compound of claim 6, wherein:
A and E are each independently C or N;
R¹ and R² are each independently H, F, Cl, Br, I or (C₁-C₆)alkyl;
R³ is H, F, Cl, Br, I or (C₁-C₆)alkyl;
R⁴, R⁵ and R⁶ are each independently H, F, Cl, Br, I, (C₁-C₆)alkyl, CF₃ or OCHF₂;
R⁷ is H or (C₁-C₆)alkyl; and
R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜ(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂).(C₆-C₁₀)aryl, (CH₂)ₜ(3-10 membered heterocyclyl) or (CH₂)ₜC(═O)(CH₂)ᵤ(3-10 membered heterocyclyl); or
R⁷ and R⁸ form a fused pyridinyl ring.

8. The compound of claim 1, having formula IV: 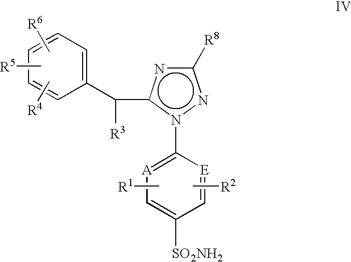
or a pharmaceutically acceptable salt or solvate thereof, wherein:
A and E are each independently C or N;
R¹ and R² are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R³ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁴, R⁵ and R⁶ are each independently H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁸ is H, F, Cl, Br, I or (C₁-C₆)alkyl; (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁶, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R⁹ is H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜO(CH₂)ᵤ(C₁-C₁₂)alkyl, (CH₂)ₜCN, (CH₂)ₜCF₃, (CF₂)ₜCF₃, (CH₂)ₜOCF₃, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁰ and R¹¹ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹²R¹³, (CH₂)ₜNR¹²R¹³, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹² and R¹³ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁴R¹⁵, (CH₂)ₜNR¹⁴R¹⁵, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹⁴ and R¹⁵ are each independently H, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜ(C₃-C₁₂)cycloalkyl(C₆-C₁₀)aryl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)N H₂, (CH₂)ₜC(═O)NH(C₁-C₁₂)alkyl, (CH₂)ₜC(═O)N((C₁-C₁₂)alkyl)₂, (CH₂)ₜNH₂, (CH₂)ₜNH(C₁-C₁₂)alkyl, (CH₂)ₜN((C₁-C₁₂)alkyl)₂, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl);
R¹, R², R³, R⁴, R⁵, R⁶, R⁷, R⁸, R⁹, R¹⁰, R¹¹, R¹², R¹³, R¹⁴ and R¹⁵ groups are each optionally independently substituted with 1 to 5 R¹⁶ groups;
R¹⁶ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl), wherein each R16 group is optionally independently substituted with 1 to 3 groups selected from H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜO(CH₂)ᵤ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, —OCHF₂, (CH₂)ₜCF₃, (CF₂)ₜCF₃, (CH₂)ₜOCF₃, SO₂(C₁-C₁₂)alkyl, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)ₜ(C₆-C₁₀)aryl, (CH₂)ₜO(CH₂)ᵤ(C₆-C₁₀)aryl, (CH₂)ₜ(3-10 membered heterocyclyl) and (CH₂)ₜC(═O)(CH₂)ᵤ(3-10 membered heterocyclyl);
t, u and v are each independently 0, 1, 2, 3, 4, 5 or 6; and
w is 1, 2 or 3.

9. The compound of claim 8, wherein:
A and E are each independently C or N;
R¹ and R² are each independently H, F, Cl, Br, I or (C₁-C₆)alkyl;
R³ is H, F, Cl, Br, I or (C₁-C₆)alkyl;
R⁴, R⁵ and R⁶ are each independently H, F, Cl, Br, I, (C₁-C₆)alkyl, CF₃ or OCHF₂; and
R⁸ is H, F, Cl, Br, I, (C₁-C₁₂)alkyl, (C₂-C₁₂)alkenyl, (C₂-C₁₂)alkynyl, (CH₂)ₜ(C₃-C₁₂)cycloalkyl, (CH₂)ₜ(C₅-C₁₂)cycloalkenyl, (CH₂)ₜ(C₈-C₁₂)cycloalkynyl, (CH₂)ₜCN, (CH₂)ₜCF₃, (CF₂)ₜCF₃, (CH₂)ₜOCF₃, (CH₂)ₜOC(═O)R⁹, (CH₂)ₜC(═O)OR⁹, (CH₂)ₜC(═O)R⁹, (CH₂)ₜOR⁹, (CH₂)ₜSO₂R⁹, (CH₂)ₜC(═O)NR¹⁰R¹¹, (CH₂)ₜNR¹⁰R¹¹, (CH₂)_{w}(C₆-C₁₀)aryl, (CH₂)ₜO(CH₂)ᵤ(C₆-C₁₀)aryl, (CH₂)ₜ(3-10 membered heterocyclyl) or (CH₂)ₜC(═O)(CH₂)ᵤ(3-10 membered heterocyclyl).

10. The compound of claim 1, having formula:

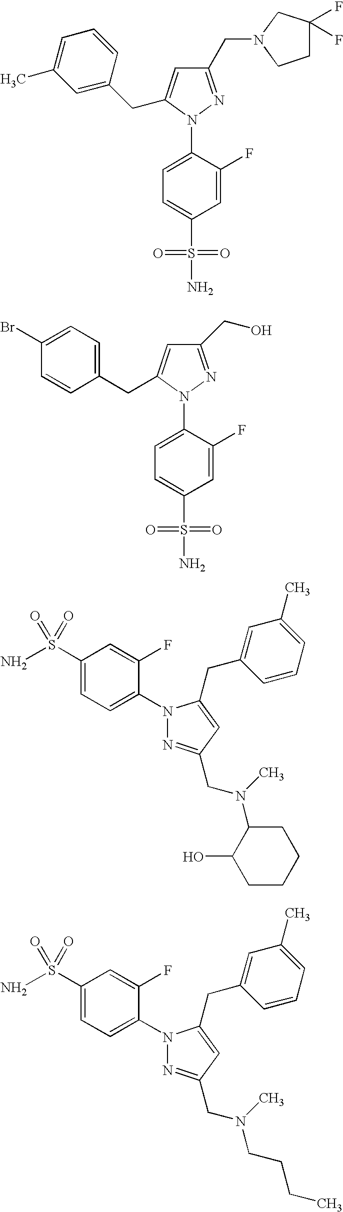

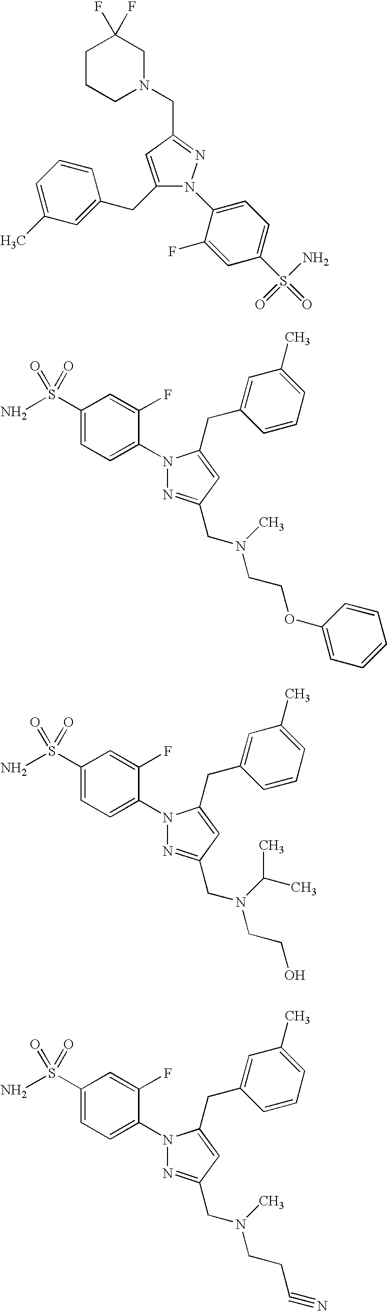

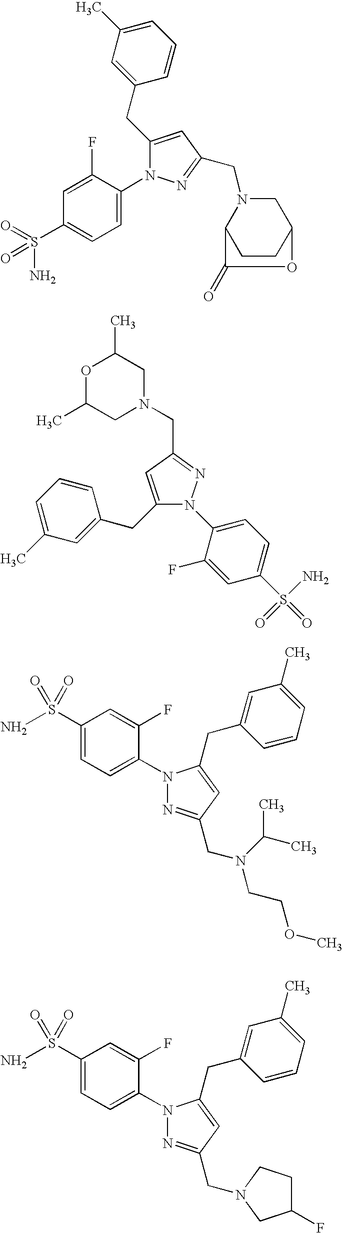

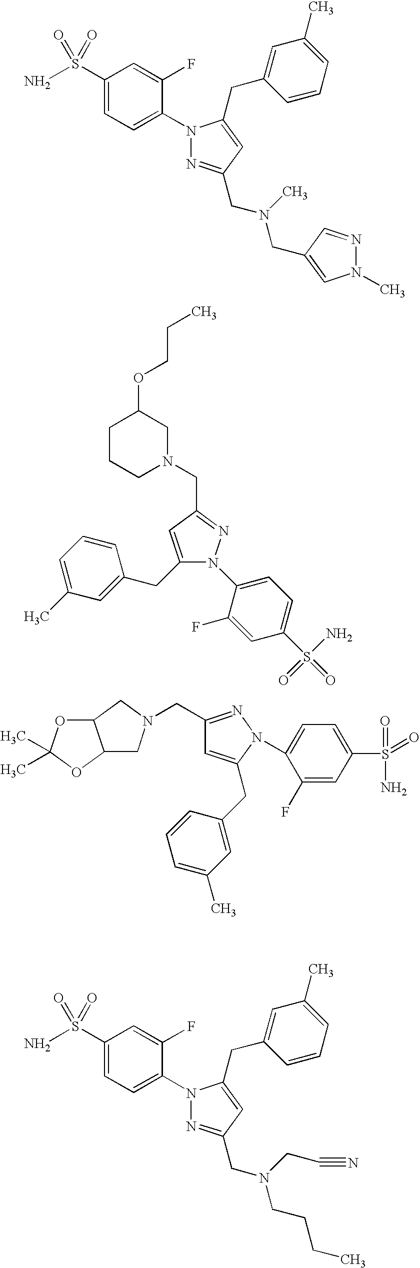

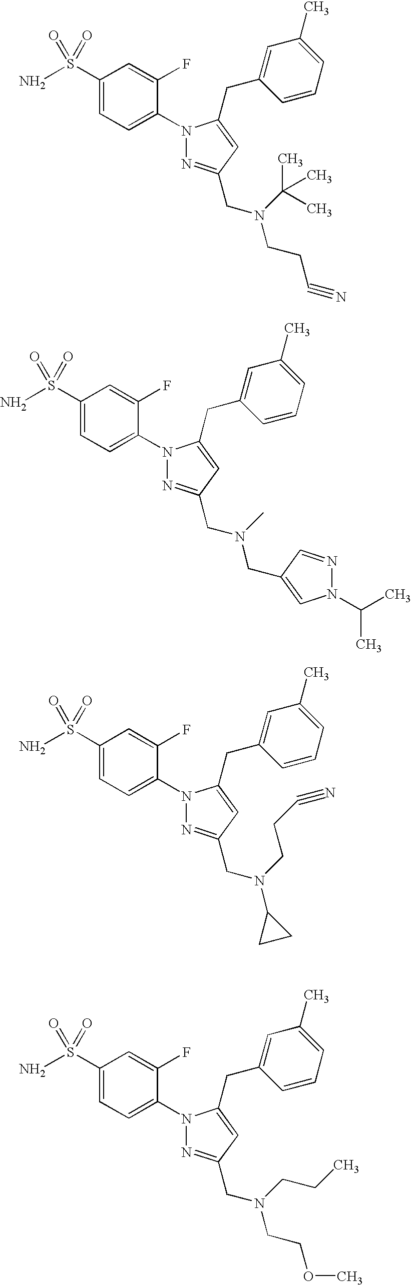

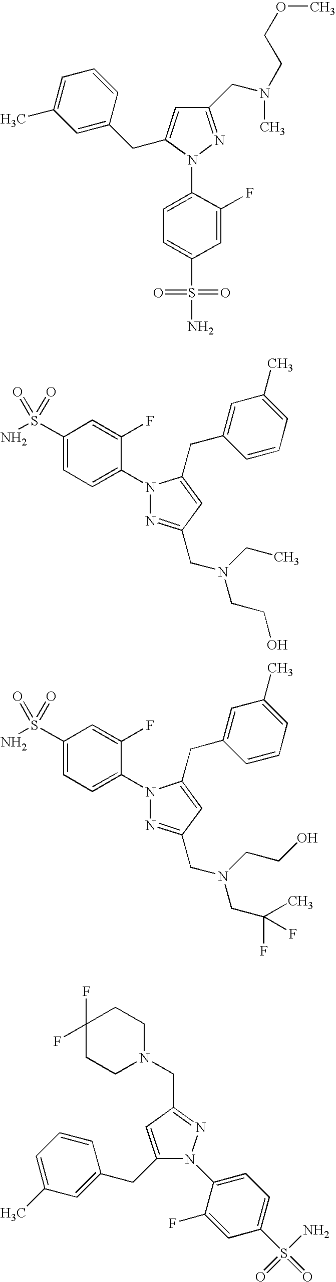

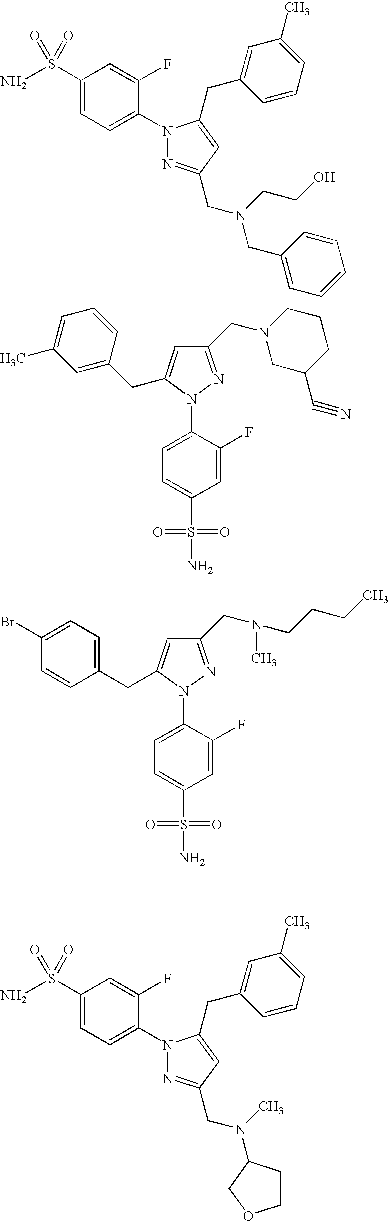

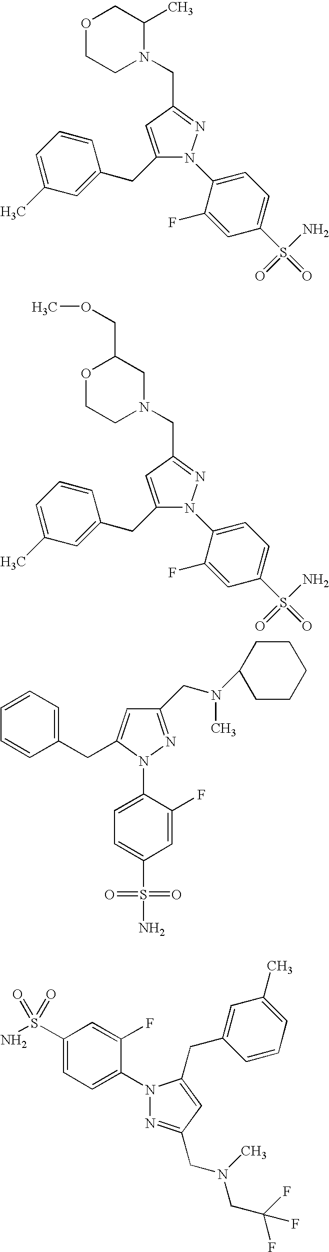

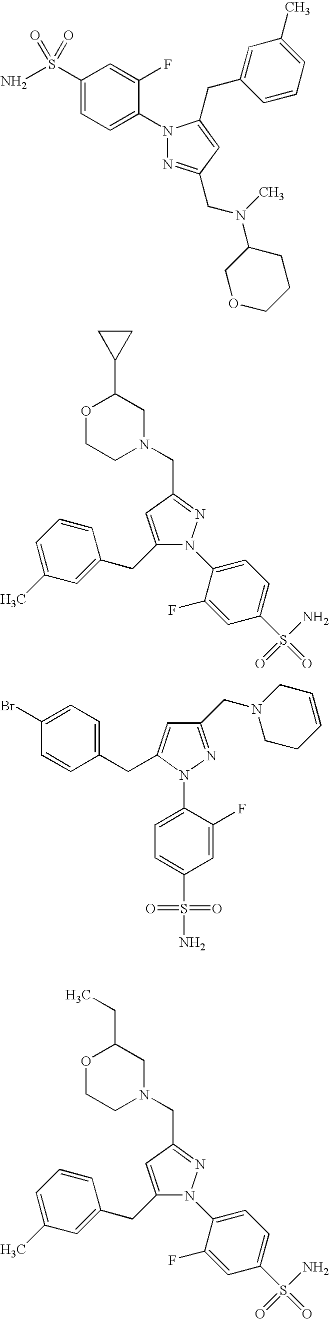

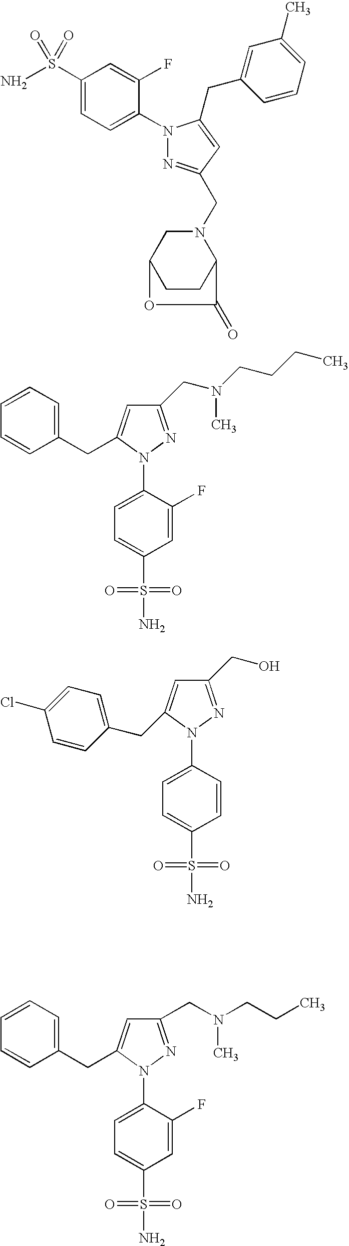

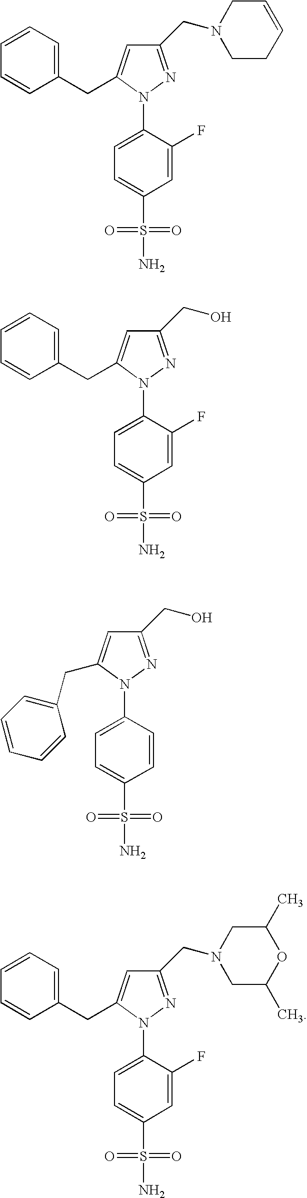

11. The compound of claim 1, having formula:

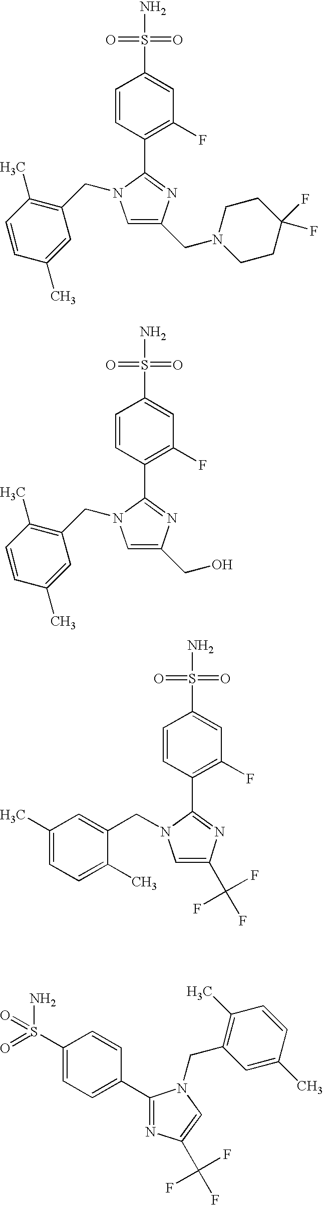

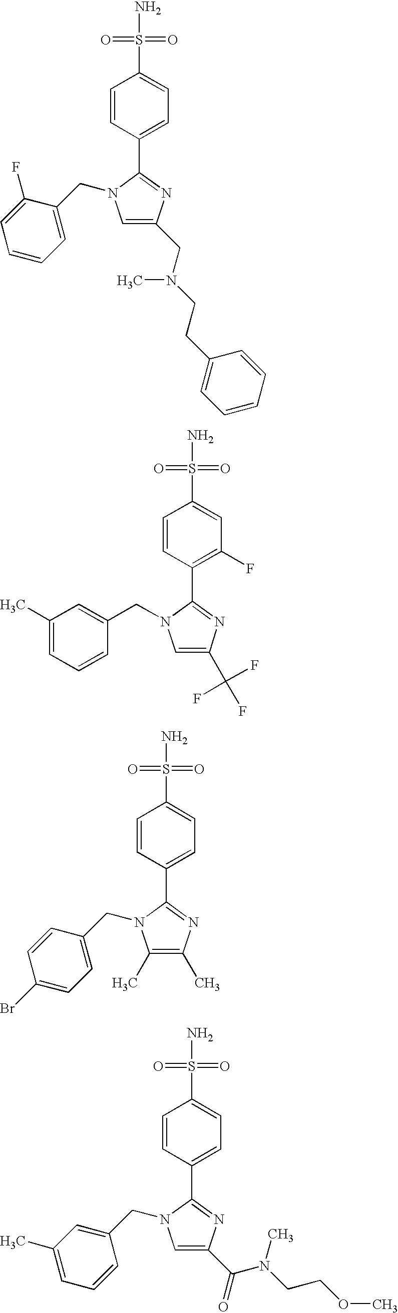

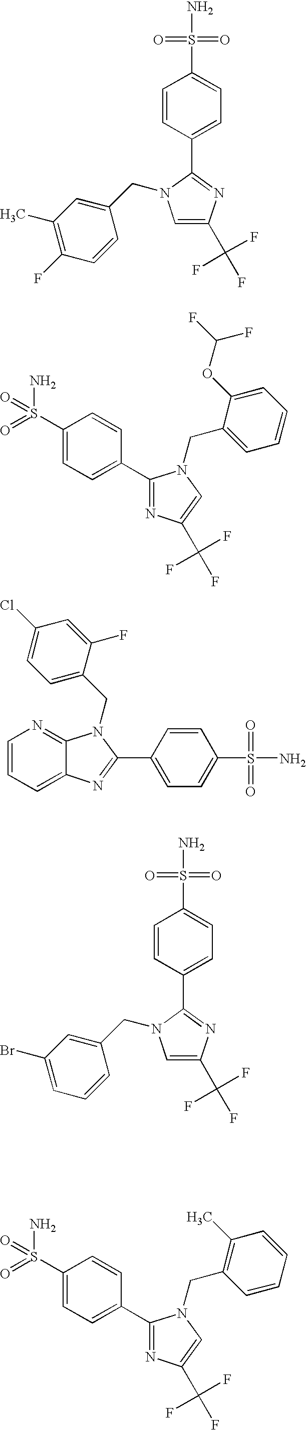

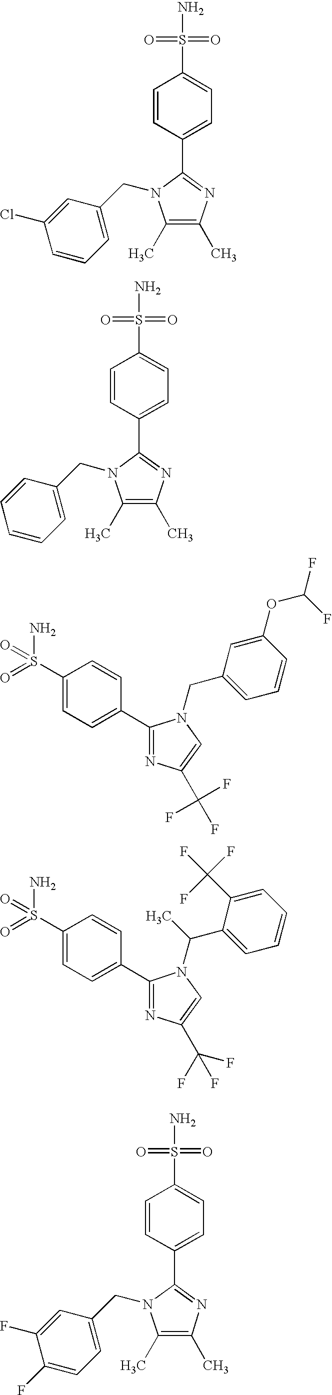

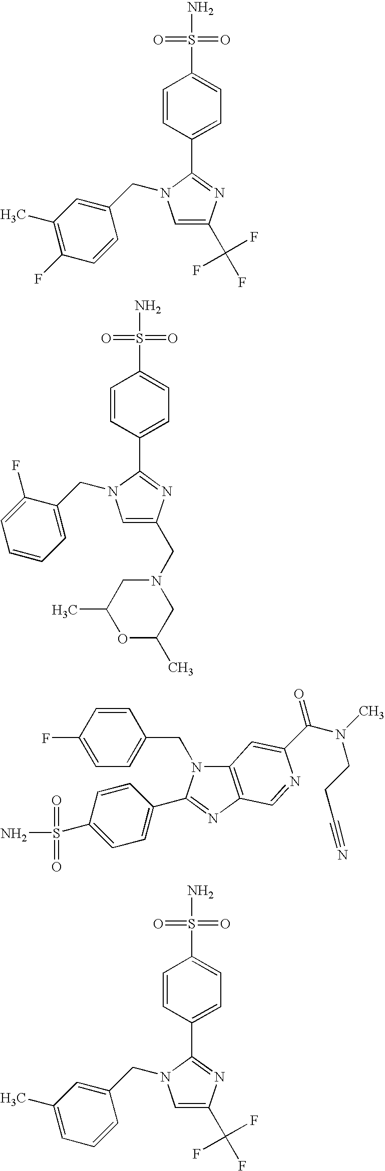

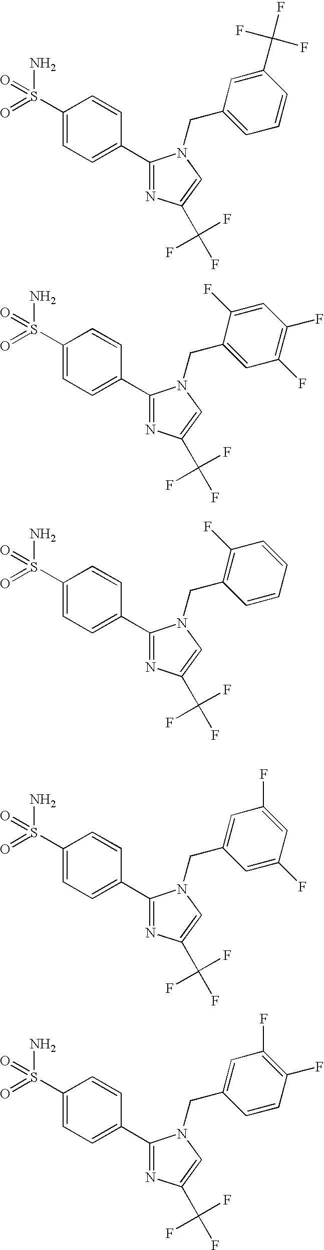

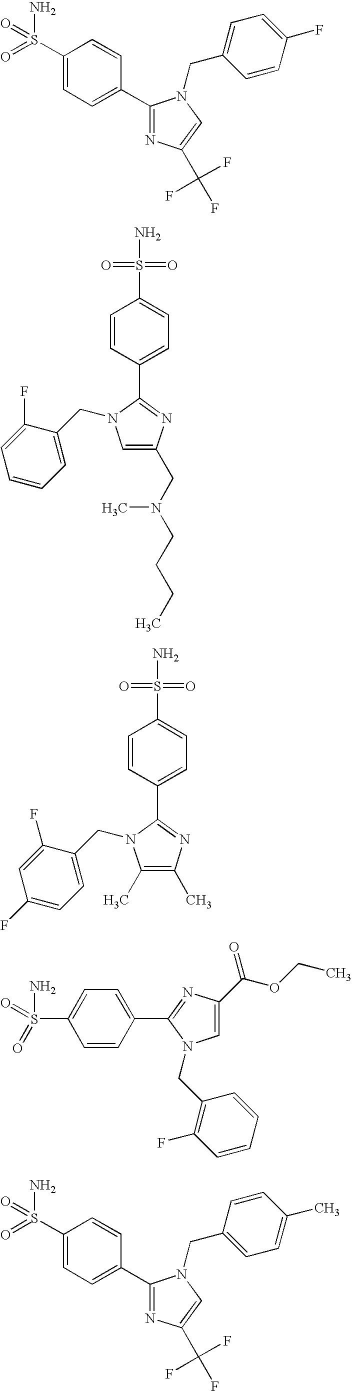

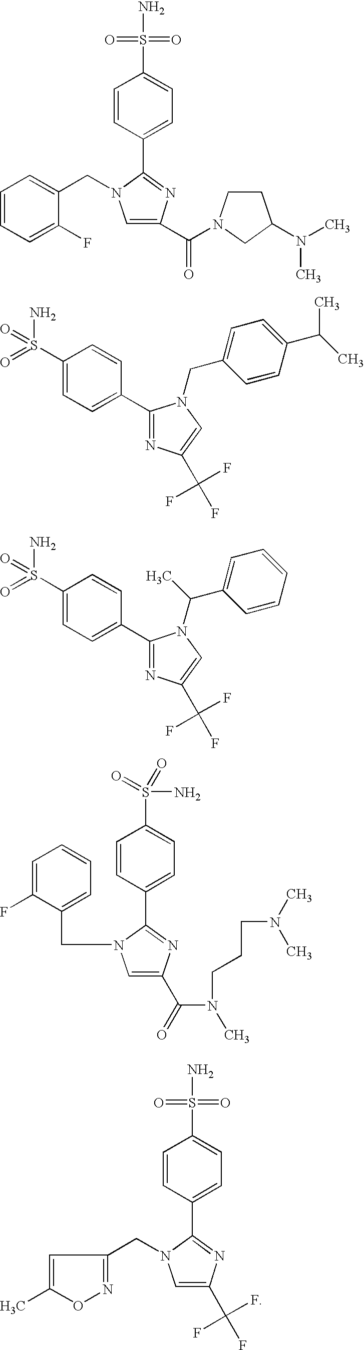

12. The compound of claim 1, having formula:

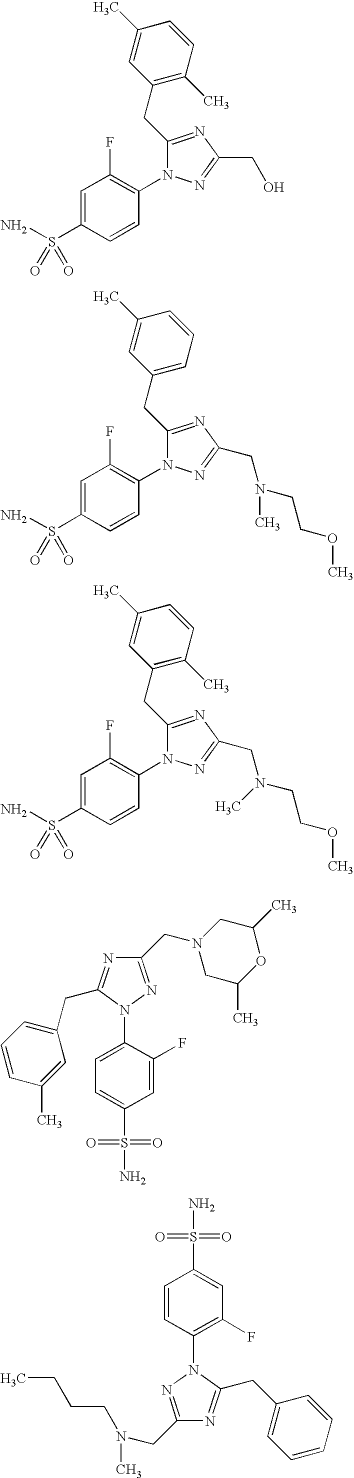

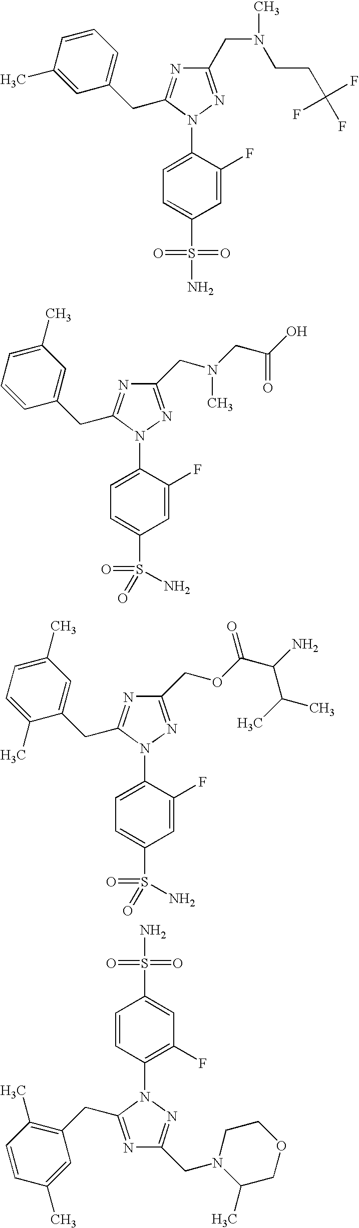

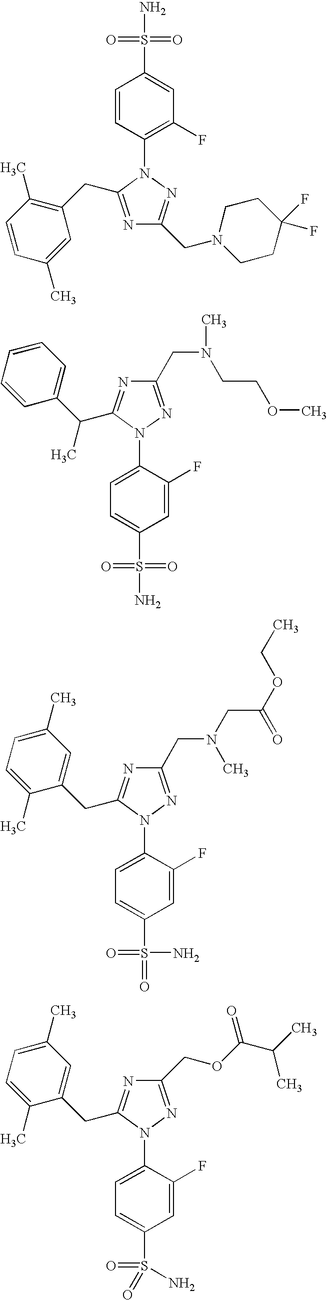

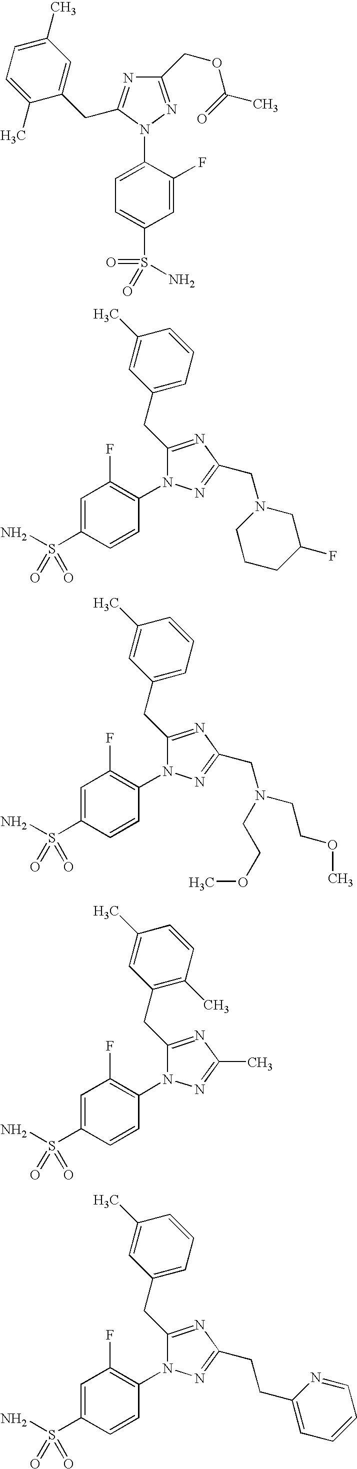

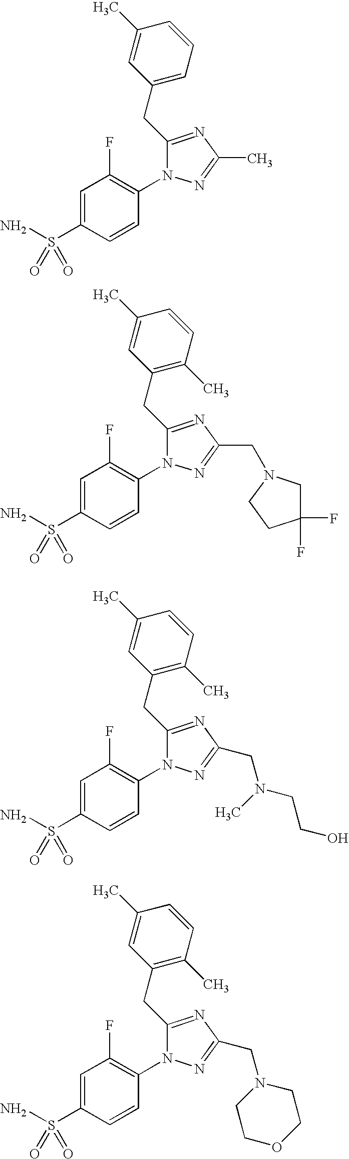

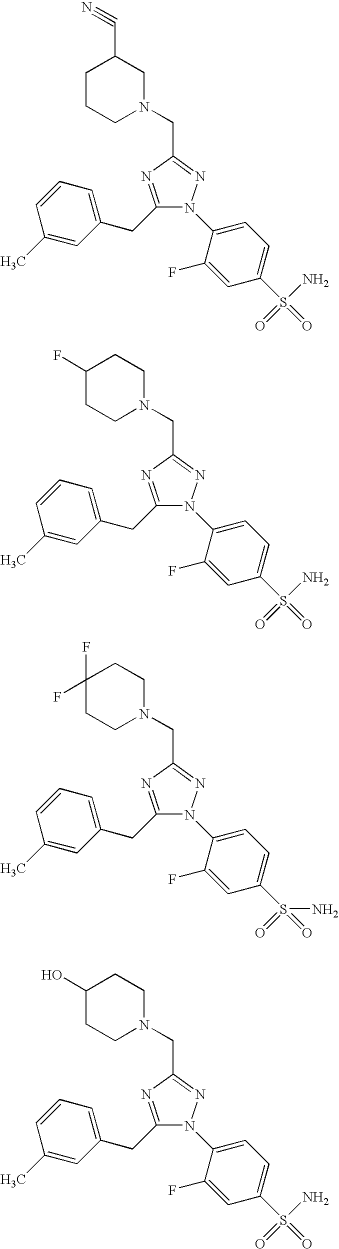

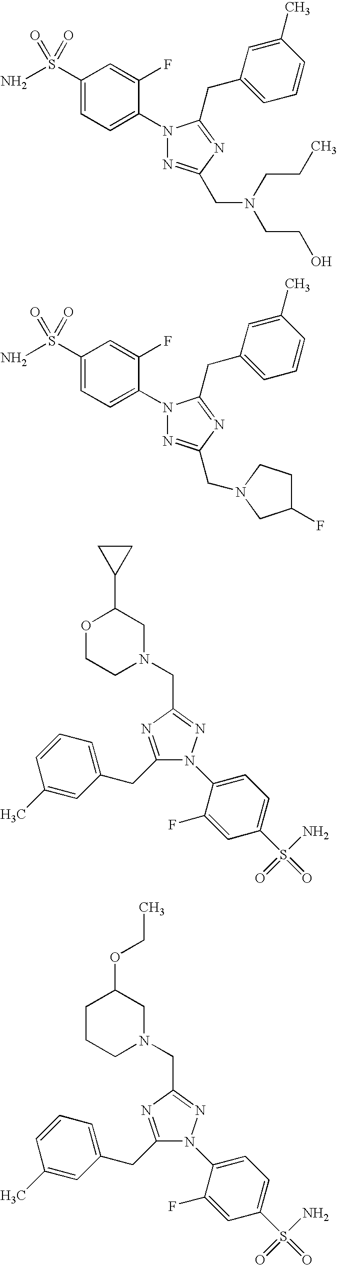

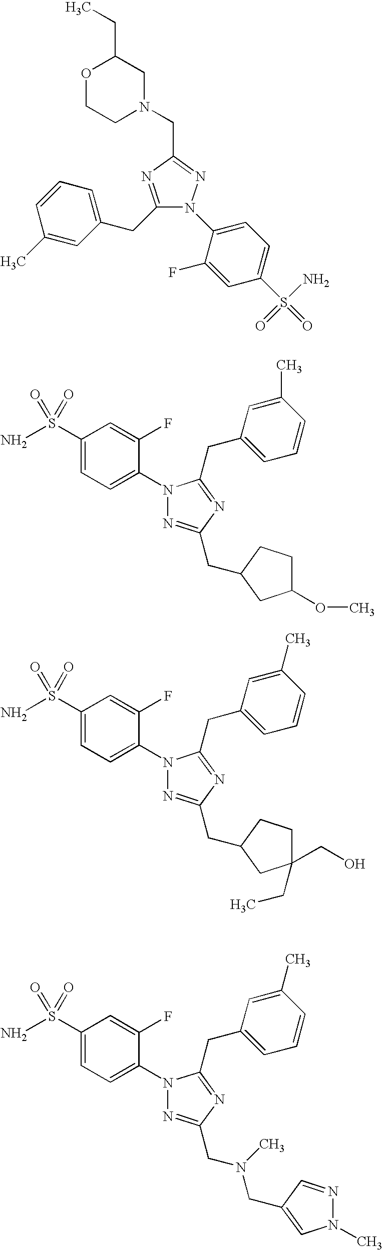

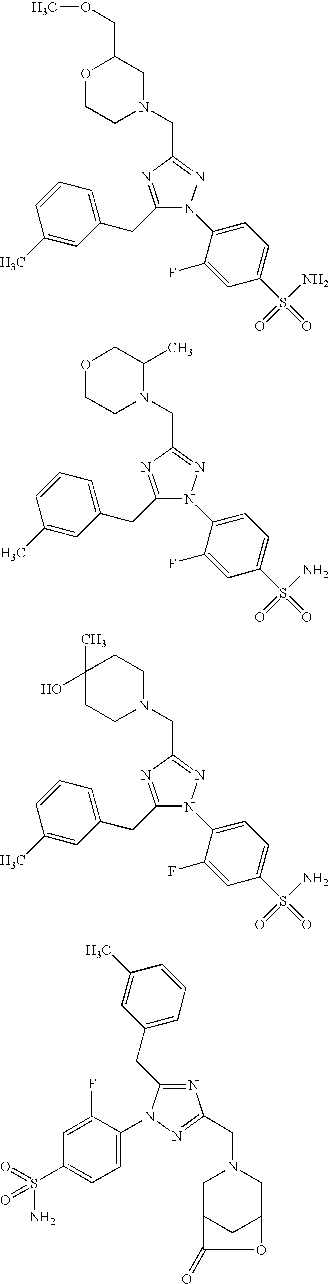

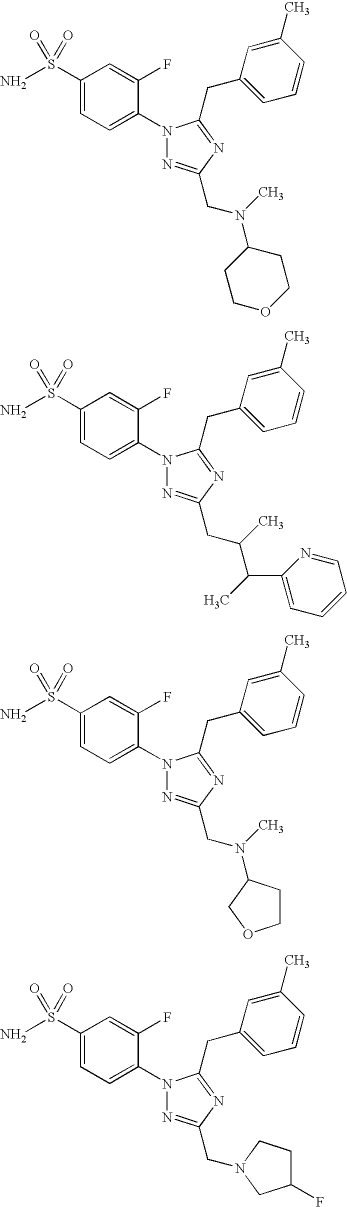

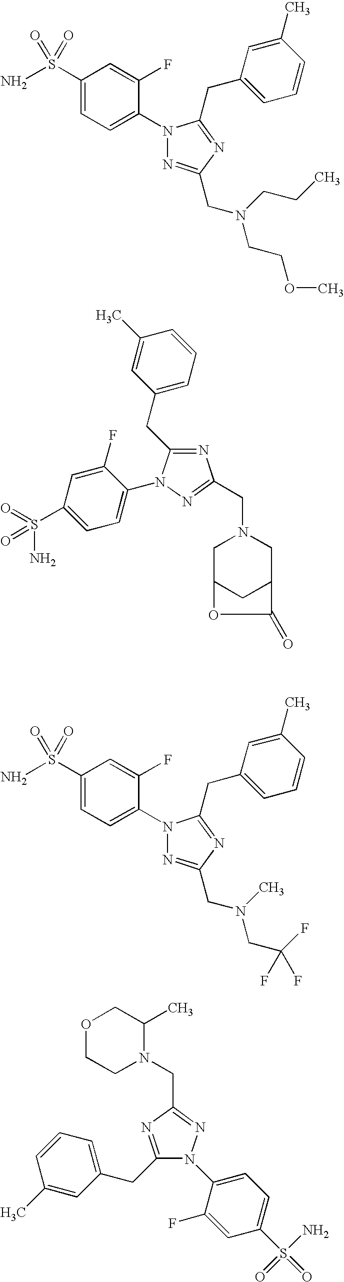

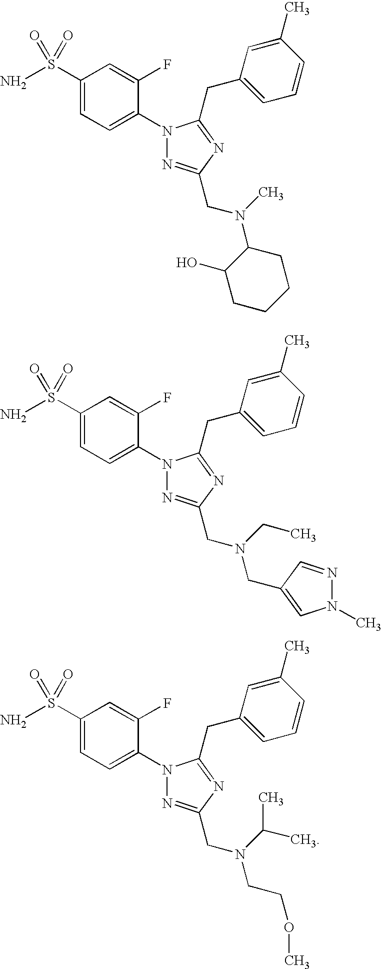

13. The compound of claim 1, having formula XIII:

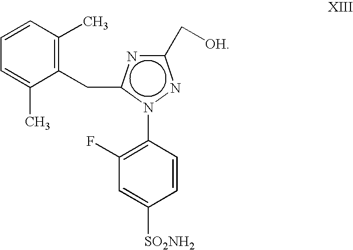

14. A process for preparing a compound of formula IV according to claim 8, the process comprising:
i) reacting a compound of formula (V) with a compound of formula (VI) and with a compound of formula (VII) in the presence of base to provide a compound of formula (VII), wherein R¹⁷ is (C₁-C₁₂)alkyl, (CH₂)ₜ(C₆-C₁₀)aryl or (CH₂)ₜ(3-10 membered heterocyclyl); and
ii) transforming the compound of formula (VII) to the compound of formula (VI).

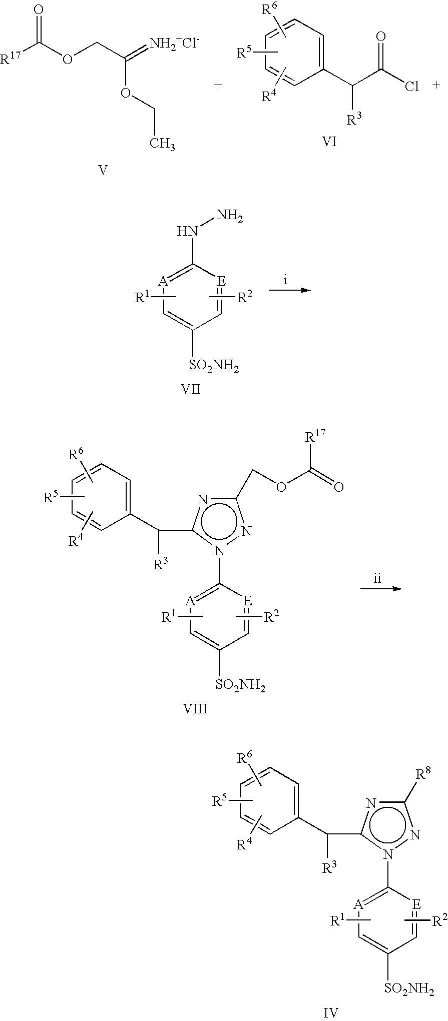

15. The compound of formula XIII: 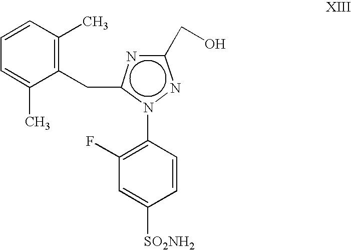
prepared by the process of claim 14.

16-20. (canceled)
